SEQUENCE LISTING (199 sequences)

SEQ ID NO: 1 - DNA, 467 bp, Artificial Sequence
  source: /note=“Description of Artificial Sequence Synthetic polynucleotide”
atagtgagtcgtattaacgtaccaacaacaaggaagtgctaaagaaacttgttctttagcacttccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 2 - DNA, 465 bp, Artificial Sequence
  source: /note=“Description of Artificial Sequence Synthetic polynucleotide”
atagtgagtcgtattaacgtaccaacaacaaggagtgctaaagaaacttgttctttagcactccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 3 - DNA, 615 bp, Artificial Sequence
  source: /note=“Description of Artificial Sequence Synthetic polynucleotide”
atagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 4 - DNA, 91 bp, Artificial Sequence
  source: /note=“Description of Artificial Sequence Synthetic oligonucleotide”
atagtgagtcgtattaacgtaccaacaacaaggaagtgctaaagaaacttgttctttagcacttccttgtttatcttagaggcatatccct

SEQ ID NO: 5 - DNA, 239 bp, Artificial Sequence
  source: /note=“Description of Artificial Sequence Synthetic polynucleotide”
atagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccct

SEQ ID NO: 6 - DNA, 473 bp, Artificial Sequence
  source: /note=“Description of Artificial Sequence Synthetic polynucleotide”
atagtgagtcgtattaacgtaccaacaagaaagatgataagcccactctacttgagagtgggcttatcatctttctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 7 - DNA, 629 bp, Artificial Sequence
  source: /note=“Description of Artificial Sequence Synthetic polynucleotide”
atagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccct

SEQ ID NO: 8 - DNA, 978 bp, Artificial Sequence
  source: /note=“Description of Artificial Sequence Synthetic polynucleotide”
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctacgtaccaacaagcaatgaggaccctgagagatacttgatctctcagggtcctcattgctttatcttagaggcatatccctacgtaccaacaagctgatgggaacgtggactaacttgtagtccacgttcccatcagctttatcttagaggcatatccctacgtaccaacaaggtcctcagattactacaaacttgttgtagtaatctgaggacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 9 - DNA, 2808 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaacaaggaagtgctaaagaaacttgttctttagcacttccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 2806 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaacaaggagtgctaaagaaacttgttctttagcactccttgtttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 2956 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - DNA, 2450 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatcaacgagctcatagtgagtcgtattaacgtaccaacaacaaggaagtgctaaagaaacttgttctttagcacttccttgtttatcttagaggcatatccctggtaccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - DNA, 2607 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgaagggcgcgccaatagtgagtcgtattaacgtaccaacaacaaggagtgctaaagaaacttgttctttagcactccttgtttatcttagaggcatatccctacgtaccaacaagagagtgattgagagtggacttgccactctcaatcactctctttatcttagaggcatatccctacgtaccaacaagagagctctgtctggaccacttgggtccagacagagctctctttatcttagaggcatatccctttttaattaacaacctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 2814 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaagaaagatgataagcccactctacttgagagtgggcttatcatctttctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctagtttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 2970 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctagtttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - DNA, 3319 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctacgtaccaacaagcaatgaggaccctgagagatacttgatctctcagggtcctcattgctttatcttagaggcatatccctacgtaccaacaagctgatgggaacgtggactaacttgtagtccacgttcccatcagctttatcttagaggcatatccctacgtaccaacaaggtcctcagattactacaaacttgttgtagtaatctgaggacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 20 bp, Artificial Sequence
  source: /note=“Description of Artificial Sequence Synthetic primer”
gctgcaaggcgattaagttg

SEQ ID NO: 18 - DNA, 143 bp, Artificial Sequence
  source: /note=“Description of Artificial Sequence Synthetic primer”
  source: /note=“Description of Combined DNA/RNA Molecule Synthetic primer”
uuutttttttttttttttttttttttttttttttttttttttttttttttttttttttttttttttttttttttttttttttttttttttttttttttttttttttttttttttttttttcagctatgaccatgttaatgcag

SEQ ID NO: 19 - DNA, 210 bp, Homo sapiens
ggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcc

SEQ ID NO: 20 - protein, 19 aa, Artificial Sequence
  source: /note=“Description of Artificial Sequence Synthetic peptide”
MLILLLPLLLFKCFCDFLK

SEQ ID NO: 21 - DNA, 57 bp, Artificial Sequence
  source: /note=“Description of Artificial Sequence Synthetic oligonucleotide”
atgctgattctgctgctgcccctgctgctgttcaagtgcttctgcgacttcctgaaa

SEQ ID NO: 22 - protein, 18 aa, Artificial Sequence
  source: /note=“Description of Artificial Sequence Synthetic peptide”
MLFYLALCLLTFTSSATA

SEQ ID NO: 23 - DNA, 54 bp, Artificial Sequence
  source: /note=“Description of Artificial Sequence Synthetic oligonucleotide”
atgctgttctatctggccctgtgcctgctgacctttaccagctctgctaccgcc

SEQ ID NO: 24 - DNA, 77 bp, Artificial Sequence
  source: /note=“Description of Artificial Sequence Synthetic oligonucleotide”
aacaaagcaccagtggtctagtggtagaatagtaccctgccacggtacagacccgggttcgattcccggctggtgca

SEQ ID NO: 25 - DNA, 17 bp, Artificial Sequence
  source: /note=“Description of Artificial Sequence Synthetic oligonucleotide”
taatacgactcactata

SEQ ID NO: 26 - RNA, 6 nt, Artificial Sequence
  source: /note=“Description of Artificial Sequence Synthetic oligonucleotide”
gccacc

SEQ ID NO: 27 - protein, 5 aa, Artificial Sequence
  source: /note=“Description of Artificial Sequence Synthetic peptide”
GGGGS

SEQ ID NO: 28 - DNA, 15 bp, Artificial Sequence
  source: /note=“Description of Artificial Sequence Synthetic oligonucleotide”
gggggtggaggctct

SEQ ID NO: 29 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagccctgagaaaggagacatgtacttgacatgtctcctttctcagggctttatcttagaggcatatccctacgtaccaacaagaggagacttgcctggtgaaaacttgtttcaccaggcaagtctcctctttatcttagaggcatatccctacgtaccaacaagagggctcttcggcaaatgtaacttgtacatttgccgaagagccctctttatcttagaggcatatcccttttatcttagaggcatatccct

SEQ ID NO: 30 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagaggagacttgcctggtgaaaacttgtttcaccaggcaagtctcctctttatcttagaggcatatcccttttatcttagaggcatatccct

SEQ ID NO: 31 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagtgaggaagtttcagaacagtacttgactgttctgaaacttcctcactttatcttagaggcatatccctacgtaccaacaagaacggtcaaagacattcacaacttgtgtgaatgtctttgaccgttctttatcttagaggcatatccctacgtaccaacaagggaaggttacatcagatcatacttgatgatctgatgtaaccttccctttatcttagaggcatatcccttttatcttagaggcatatccct

SEQ ID NO: 32 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagtgaggaagtttcagaacagtacttgactgttctgaaacttcctcactttatcttagaggcatatcccttttatcttagaggcatatccct

SEQ ID NO: 33 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagggaaggttacatcagatcatacttgatgatctgatgtaaccttccctttatcttagaggcatatcccttttatcttagaggcatatccct

SEQ ID NO: 34 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagcagctgaggccattatatgaacttgtcatataatggcctcagctgctttatcttagaggcatatccctacgtaccaacaaggacccaggaaatgttcagaaacttgttctgaacatttcctgggtcctttatcttagaggcatatccctacgtaccaacaaggctgaaagaccagaacaagaacttgtcttgttctggtctttcagcctttatcttagaggcatatcccttttatcttagaggcatatccct

SEQ ID NO: 35 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagcagctgaggccattatatgaacttgtcatataatggcctcagctgctttatcttagaggcatatcccttttatcttagaggcatatccct

SEQ ID NO: 36 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagtgtgaccgaaaggtaagatgacttgcatcttacctttcggtcacactttatcttagaggcatatccctacgtaccaacaagaggtgatgaagtcagacaaaacttgtttgtctgacttcatcacctctttatcttagaggcatatccctacgtaccaacaagcaactgagggagccttgaatacttgattcaaggctccctcagttgctttatcttagaggcatatcccttttatcttagaggcatatccct

SEQ ID NO: 37 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagaggtgatgaagtcagacaaaacttgtttgtctgacttcatcacctctttatcttagaggcatatcccttttatcttagaggcatatccct

SEQ ID NO: 38 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagcaactgagggagccttgaatacttgattcaaggctccctcagttgctttatcttagaggcatatcccttttatcttagaggcatatccct

SEQ ID NO: 39 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagttgctgattattctgtcctaacttgtaggacagaataatcagcaactttatcttagaggcatatccctacgtaccaacaagaggtgatgaagtcagacaaaacttgtttgtctgacttcatcacctctttatcttagaggcatatccctacgtaccaacaagccggtagcacaccttgtaatacttgattacaaggtgtgctaccggctttatcttagaggcatatcccttttatcttagaggcatatccct

SEQ ID NO: 40 - DNA, 839 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaatttaaatattgggatcagacacttggtctgatcccaatatttaaatttatcttagaggcatatccctacgtaccaacaaaagaatagagctcgcacacttggtgcgagctctattctttttatcttagaggcatatccctacgtaccaacaaactgttgattcatcacagggacttgccctgtgatgaatcaacagttttatcttagaggcatatcccttttatcttagaggcatatccct

SEQ ID NO: 41 - DNA, 681 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaaaagaatagagctcgcacacttggtgcgagctctattctttttatcttagaggcatatcccttttatcttagaggcatatccct

SEQ ID NO: 42 - DNA, 689 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagtgtgaccgaaaggtaagatgacttgcatcttacctttcggtcacactttatcttagaggcatatcccttttatcttagaggcatatccct

SEQ ID NO: 43 - DNA, 851 bp, Artificial Sequence
  source: /note=“Description of Artificial Sequence Synthetic polynucleotide”
gccaccatgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagaggagacttgcctggtgaaaacttgtttcaccaggcaagtctcctctttatcttagaggcatatccctacgtaccaacaagcagctgaggccattatatgaacttgtcatataatggcctcagctgctttatcttagaggcatatccctacgtaccaacaagcaactgagggagccttgaatacttgattcaaggctccctcagttgctttatcttagaggcatatcccttttatcttagaggcatatccct

SEQ ID NO: 44 - DNA, 851 bp, Artificial Sequence
  source: /note=“Description of Artificial Sequence Synthetic polynucleotide”
gccaccatgctcctgatctgcctgctggtgattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagaggagacttgcctggtgaaaacttgtttcaccaggcaagtctcctctttatcttagaggcatatccctacgtaccaacaagcagctgaggccattatatgaacttgtcatataatggcctcagctgctttatcttagaggcatatccctacgtaccaacaagcaactgagggagccttgaatacttgattcaaggctccctcagttgctttatcttagaggcatatcccttttatcttagaggcatatccct

SEQ ID NO: 45 - DNA, 851 bp, Artificial Sequence
  source: /note=“Description of Artificial Sequence Synthetic polynucleotide”
gccaccatgctcctgaagctcctgctggtgattgccctgctggcctgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactgaatagtgagtcgtattaacgtaccaacaagaggagacttgcctggtgaaaacttgtttcaccaggcaagtctcctctttatcttagaggcatatccctacgtaccaacaagcagctgaggccattatatgaacttgtcatataatggcctcagctgctttatcttagaggcatatccctacgtaccaacaagcaactgagggagccttgaatacttgattcaaggctccctcagttgctttatcttagaggcatatcccttttatcttagaggcatatccct

SEQ ID NO: 46 - DNA, 2510 bp, Artificial Sequence
  source: /note=“Description of Artificial Sequence Synthetic polynucleotide”
gccaccatgtctagcagctcttggctgctgctgtctctggtggctgtgacagccgctcagagcaccattgaggaacaggccaagaccttcctggacaagttcaaccacgaggccgaggacctgttctaccagtctagcctggccagctggaactacaacaccaacatcaccgaagagaacgtgcagaacatgaacaacgccggcgacaagtggagcgccttcctgaaagagcagagcacactggcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgccatggtggatcaggcttgggatgcccagcggatcttcaaagaggccgagaagttcttcgtgtccgtgggcctgcctaatatgacccaaggcttctgggagaactccatgctgacagaccccggcaatgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccacgaagccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaatatgaacgtgcggccactgctgaactacttcgagcccctgttcacctggctgaaggaccagaacaagaacagcttcgtcggctggtccaccgattggagcccttacgccgaccagagcatcaaagtgcggatcagcctgaaaagcgccctgggcgataaggcctatgagtggaacgacaatgagatgtacctgttccggtccagcgtggcctatgctatgcggcagtactttctgaaagtcaagaaccagatgatcctgttcggcgaagaggatgtgcgcgtggccaacctgaagcctcggatcagcttcaacttcttcgtgactgcccctaagaacgtgtccgacatcatccccagaaccgaggtggaaaaggccatcagaatgagcagaagccggatcaacgacgccttccggctgaacgacaactccctggaattcctgggcattcagcccacactgggccctccaaatcagcctcctgtgtcctaaatagtgagtcgtattaacgtaccaacaagtgtgaccgaaaggtaagatgacttgcatcttacctttcggtcacactttatcttagaggcatatccctacgtaccaacaagaggtgatgaagtcagacaaaacttgtttgtctgacttcatcacctctttatcttagaggcatatccctacgtaccaacaagcaactgagggagccttgaatacttgattcaaggctccctcagttgctttatcttagaggcatatcccttttatcttagaggcatatccct

SEQ ID NO: 47 - DNA, 2510 bp, Artificial Sequence
  source: /note=“Description of Artificial Sequence Synthetic polynucleotide”
gccaccatgtctagcagctcttggctgctgctgtctctggtggctgtgacagccgctcagagcaccattgaggaacaggccaagaccttcctggacaagttcaaccacgaggccgaggacctgttctaccagtctagcctggccagctggaactacaacaccaacatcaccgaagagaacgtgcagaacatgaacaacgccggcgacaagtggagcgccttcctgaaagagcagagcacactggcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgccatggtggatcaggcttgggatgcccagcggatcttcaaagaggccgagaagttcttcgtgtccgtgggcctgcctaatatgacccaaggcttctgggagaactccatgctgacagaccccggcaatgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccacgaagccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaatatgaacgtgcggccactgctgaactacttcgagcccctgttcacctggctgaaggaccagaacaagaacagcttcgtcggctggtccaccgattggagcccttacgccgaccagagcatcaaagtgcggatcagcctgaaaagcgccctgggcgataaggcctatgagtggaacgacaatgagatgtacctgttccggtccagcgtggcctatgctatgcggcagtactttctgaaagtcaagaaccagatgatcctgttcggcgaagaggatgtgcgcgtggccaacctgaagcctcggatcagcttcaacttcttcgtgactgcccctaagaacgtgtccgacatcatccccagaaccgaggtggaaaaggccatcagaatgagcagaagccggatcaacgacgccttccggctgaacgacaactccctggaattcctgggcattcagcccacactgggccctccaaatcagcctcctgtgtcctaaatagtgagtcgtattaacgtaccaacaagttgctgattattctgtcctaacttgtaggacagaataatcagcaactttatcttagaggcatatccctacgtaccaacaagaggtgatgaagtcagacaaaacttgtttgtctgacttcatcacctctttatcttagaggcatatccctacgtaccaacaagccggtagcacaccttgtaatacttgattacaaggtgtgctaccggctttatcttagaggcatatcccttttatcttagaggcatatccct

SEQ ID NO: 48 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 49 - DNA, 859 bp, Homo sapiens
ccatacccatggagaaaggacattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 50 - DNA, 564 bp, Artificial Sequence
  source: /note=“Description of Artificial Sequence Synthetic polynucleotide”
atgaccaacaagtgcctgctgcagattgccctgctgctgtgcttcagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaactga

SEQ ID NO: 51 - protein, 21 aa, Homo sapiens
MTNKCLLQIALLLCFSTTALS

SEQ ID NO: 52 - protein, 21 aa, Artificial Sequence
  source: /note=“Description of Artificial Sequence Synthetic peptide”
MLLICLLVIALLLCFSTTALS

SEQ ID NO: 53 - DNA, 63 bp, Artificial Sequence
  source: /note=“Description of Artificial Sequence Synthetic oligonucleotide”
atgctcctgatctgcctgctggtgattgccctgctgctgtgcttcagcacaacagccctgagc

SEQ ID NO: 54 - protein, 21 aa, Artificial Sequence
  source: /note=“Description of Artificial Sequence Synthetic peptide”
MLLKLLLVIALLACFSTTALS

SEQ ID NO: 55 - DNA, 63 bp, Artificial Sequence
  source: /note=“Description of Artificial Sequence Synthetic oligonucleotide”
atgctcctgaagctcctgctggtgattgccctgctggcctgcttcagcacaacagccctgagc

SEQ ID NO: 56 - protein, 805 aa, Homo sapiens [sequence omitted]

SEQ ID NO: 57 - DNA, 2418 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 58 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 59 - DNA, 2223 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcctaa

SEQ ID NO: 60 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 61 - DNA, 669 bp, Severe acute respiratory syndrome coronavirus 2
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 62 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 63 - DNA, 1995 bp, Artificial Sequence
  source: /note=“Description of Artificial Sequence Synthetic polynucleotide”
gccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggccgggggtggaggctctgtgtccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagctcgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaagtga

SEQ ID NO: 64 - protein, 180 aa, Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 65 - protein, 100 aa, Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRS

SEQ ID NO: 66 - DNA, 1138 bp, Artificial Sequence
  source: /note=“Description of Artificial Sequence Synthetic polynucleotide”
gacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtgggggtggaggctctgtgtccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagctcgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaagtga

SEQ ID NO: 67 - DNA, 3749 bp, Severe acute respiratory syndrome coronavirus 2
gtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggca

SEQ ID NO: 68 - DNA, 20 bp, Artificial Sequence
  source: /note=“Description of Artificial Sequence Synthetic primer”
gatgtggtgcttgcatacgt

SEQ ID NO: 69 - DNA, 20 bp, Artificial Sequence
  source: /note=“Description of Artificial Sequence Synthetic primer”
tgctgttacgaccatgtcat

SEQ ID NO: 70 - DNA, 20 bp, Artificial Sequence
  source: /note=“Description of Artificial Sequence Synthetic primer”
tcacaacctggagcattgca

SEQ ID NO: 71 - DNA, 20 bp, Artificial Sequence
  source: /note=“Description of Artificial Sequence Synthetic primer”
aatagagctcgcaccgtagc

SEQ ID NO: 72 - DNA, 27 bp, Artificial Sequence
  source: /note=“Description of Artificial Sequence Synthetic primer”
ggtgtctctatctgtagtactatgacc

SEQ ID NO: 73 - DNA, 20 bp, Artificial Sequence
  source: /note=“Description of Artificial Sequence Synthetic primer”
agtggcggctattgatttca

SEQ ID NO: 74 - DNA, 639 bp, Unknown
  source: /note=“Description of Unknown IL-6 sequence”
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 75 - DNA, 1059 bp, Unknown
  source: /note=“Description of Unknown IL-6R-alpha sequence”
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagacaggctttctcctcgttgcccaggatggagtacagcagtgcaatcacagctcacggcaacttctgcctcctgggttcaagcaatcctcccgcctcagcctcctaagtag

SEQ ID NO: 76 - DNA, 990 bp, Unknown
  source: /note=“Description of Unknown IL-6R-beta sequence”
atgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagataacattgcctccttttga

SEQ ID NO: 77 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 78 - DNA, 29751 bp, SARS coronavirus Tor2
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 79 - DNA, 30119 bp, Middle East respiratory syndrome-related coronavirus
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 80 - DNA, 18 bp, Unknown
  source: /note=“Description of Unknown IL-8 siRNA sense sequence”
caaggaagtgctaaagaa

SEQ ID NO: 81 - DNA, 17 bp, Unknown
  source: /note=“Description of Unknown IL-8 siRNA sense sequence”
caaggagtgctaaagaa

SEQ ID NO: 82 - DNA, 18 bp, Unknown
  source: /note=“Description of Unknown IL-8 siRNA sense sequence”
gagagtgattgagagtgg

SEQ ID NO: 83 - DNA, 18 bp, Unknown
  source: /note=“Description of Unknown IL-8 siRNA sense sequence”
gagagctctgtctggacc

SEQ ID NO: 84 - DNA, 21 bp, Unknown
  source: /note=“Description of Unknown IL-1beta siRNA sense sequence”
gaaagatgataagcccactct

SEQ ID NO: 85 - DNA, 21 bp, Unknown
  source: /note=“Description of Unknown IL-1beta siRNA sense sequence”
ggtgatgtctggtccatatga

SEQ ID NO: 86 - DNA, 18 bp, Unknown
  source: /note=“Description of Unknown IL-1beta siRNA sense sequence”
gatgataagcccactcta

SEQ ID NO: 87 - DNA, 20 bp, Unknown
  source: /note=“Description of Unknown TNF-alpha sense sequence”
ggcgtggagctgagagataa

SEQ ID NO: 88 - DNA, 20 bp, Unknown
  source: /note=“Description of Unknown TNF-alpha sense sequence”
gggcctgtacctcatctact

SEQ ID NO: 89 - DNA, 19 bp, Unknown
  source: /note=“Description of Unknown TNF-alpha sense sequence”
ggtatgagcccatctatct

SEQ ID NO: 90 - DNA, 21 bp, Unknown
  source: /note=“Description of Unknown IL-17 sense sequence”
gcaatgaggaccctgagagat

SEQ ID NO: 91 - DNA, 20 bp, Unknown
  source: /note=“Description of Unknown IL-17 sense sequence”
gctgatgggaacgtggacta

SEQ ID NO: 92 - DNA, 19 bp, Unknown
  source: /note=“Description of Unknown IL-17 sense sequence”
ggtcctcagattactacaa

SEQ ID NO: 93 - DNA, 21 bp, Unknown
  source: /note=“Description of Unknown IL-6 sense sequence”
gccctgagaaaggagacatgt

SEQ ID NO: 94 - DNA, 21 bp, Unknown
  source: /note=“Description of Unknown IL-6 sense sequence”
gaggagacttgcctggtgaaa

SEQ ID NO: 95 - DNA, 21 bp, Unknown
  source: /note=“Description of Unknown IL-6 sense sequence”
gagggctcttcggcaaatgta

SEQ ID NO: 96 - DNA, 21 bp, Unknown
  source: /note=“Description of Unknown IL6R-alpha sense sequence”
gtgaggaagtttcagaacagt

SEQ ID NO: 97 - DNA, 21 bp, Unknown
  source: /note=“Description of Unknown IL6R-alpha sense sequence”
gaacggtcaaagacattcaca

SEQ ID NO: 98 - DNA, 21 bp, Unknown
  source: /note=“Description of Unknown IL6R-Beta sense sequence”
gggaaggttacatcagatcat

SEQ ID NO: 99 - DNA, 21 bp, Homo sapiens
gcagctgaggccattatatga

SEQ ID NO: 100 - DNA, 21 bp, Homo sapiens
ggacccaggaaatgttcagaa

SEQ ID NO: 101 - DNA, 21 bp, Homo sapiens
ggctgaaagaccagaacaaga

SEQ ID NO: 102 - DNA, 21 bp, Severe acute respiratory syndrome coronavirus 2
gtgtgaccgaaaggtaagatg

SEQ ID NO: 103 - DNA, 20 bp, Severe acute respiratory syndrome coronavirus 2
tttaaatattgggatcagac

SEQ ID NO: 104 - DNA, 17 bp, Severe acute respiratory syndrome coronavirus 2
aagaatagagctcgcac

SEQ ID NO: 105 - DNA, 20 bp, Severe acute respiratory syndrome coronavirus 2
actgttgattcatcacaggg

SEQ ID NO: 106 - DNA, 21 bp, Severe acute respiratory syndrome coronavirus 2
gttgctgattattctgtccta

SEQ ID NO: 107 - DNA, 21 bp, Severe acute respiratory syndrome coronavirus 2
gaggtgatgaagtcagacaaa

SEQ ID NO: 108 - DNA, 21 bp, Severe acute respiratory syndrome coronavirus 2
gccggtagcacaccttgtaat

SEQ ID NO: 109 - DNA, 21 bp, Severe acute respiratory syndrome coronavirus 2
gcaactgagggagccttgaat

SEQ ID NO: 110 - DNA, 18 bp, Unknown
  source: /note=“Description of Unknown IL-8 siRNA antisense sequence”
ttctttagcacttccttg

SEQ ID NO: 111 - DNA, 17 bp, Unknown
  source: /note=“Description of Unknown IL-8 siRNA antisense sequence”
ttctttagcactccttg

SEQ ID NO: 112 - DNA, 18 bp, Unknown
  source: /note=“Description of Unknown IL-8 siRNA antisense sequence”
ccactctcaatcactctc

SEQ ID NO: 113 - DNA, 18 bp, Unknown
  source: /note=“Description of Unknown IL-8 siRNA antisense sequence”
ggtccagacagagctctc

SEQ ID NO: 114 - DNA, 21 bp, Unknown
  source: /note=“Description of Unknown IL-1beta siRNA antisense sequence”
agagtgggcttatcatctttc

SEQ ID NO: 115 - DNA, 21 bp, Unknown
  source: /note=“Description of Unknown IL-1beta siRNA antisense sequence”
tcatatggaccagacatcacc

SEQ ID NO: 116 - DNA, 18 bp, Unknown
  source: /note=“Description of Unknown IL-1beta siRNA antisense sequence”
tagagtgggcttatcatc

SEQ ID NO: 117 - DNA, 20 bp, Unknown
  source: /note=“Description of Unknown TNF-alpha antisense sequence”
ttatctctcagctccacgcc

SEQ ID NO: 118 - DNA, 20 bp, Unknown
  source: /note=“Description of Unknown TNF-alpha antisense sequence”
agtagatgaggtacaggccc

SEQ ID NO: 119 - DNA, 19 bp, Unknown
  source: /note=“Description of Unknown TNF-alpha antisense sequence”
agatagatgggctcatacc

SEQ ID NO: 120 - DNA, 21 bp, Unknown
  source: /note=“Description of Unknown IL-17 antisense sequence”
atctctcagggtcctcattgc

SEQ ID NO: 121 - DNA, 20 bp, Unknown
  source: /note=“Description of Unknown IL-17 antisense sequence”
tagtccacgttcccatcagc

SEQ ID NO: 122 - DNA, 19 bp, Unknown
  source: /note=“Description of Unknown IL-17 antisense sequence”
ttgtagtaatctgaggacc

SEQ ID NO: 123 - DNA, 21 bp, Unknown
  source: /note=“Description of Unknown IL-6 antisense sequence”
acatgtctcctttctcagggc

SEQ ID NO: 124 - DNA, 21 bp, Unknown
  source: /note=“Description of Unknown IL-6 antisense sequence”
tttcaccaggcaagtctcctc

SEQ ID NO: 125 - DNA, 21 bp, Unknown
  source: /note=“Description of Unknown IL-6 antisense sequence”
tacatttgccgaagagccctc

SEQ ID NO: 126 - DNA, 21 bp, Unknown
  source: /note=“Description of Unknown IL6R-alpha antisense sequence”
actgttctgaaacttcctcac

SEQ ID NO: 127 - DNA, 21 bp, Unknown
  source: /note=“Description of Unknown IL6R-alpha antisense sequence”
tgtgaatgtctttgaccgttc

SEQ ID NO: 128 - DNA, 21 bp, Unknown
  source: /note=“Description of Unknown IL6R-Beta antisense sequence”
atgatctgatgtaaccttccc

SEQ ID NO: 129 - DNA, 21 bp, Homo sapiens
tcatataatggcctcagctgc

SEQ ID NO: 130 - DNA, 21 bp, Homo sapiens
ttctgaacatttcctgggtcc

SEQ ID NO: 131 - DNA, 21 bp, Homo sapiens
tcttgttctggtctttcagcc

SEQ ID NO: 132 - DNA, 21 bp, Severe acute respiratory syndrome coronavirus 2
catcttacctttcggtcacac

SEQ ID NO: 133 - DNA, 20 bp, Severe acute respiratory syndrome coronavirus 2
gtctgatcccaatatttaaa

SEQ ID NO: 134 - DNA, 17 bp, Severe acute respiratory syndrome coronavirus 2
gtgcgagctctattctt

SEQ ID NO: 135 - DNA, 20 bp, Severe acute respiratory syndrome coronavirus 2
ccctgtgatgaatcaacagt

SEQ ID NO: 136 - DNA, 21 bp, Severe acute respiratory syndrome coronavirus 2
taggacagaataatcagcaac

SEQ ID NO: 137 - DNA, 21 bp, Severe acute respiratory syndrome coronavirus 2
tttgtctgacttcatcacctc

SEQ ID NO: 138 - DNA, 21 bp, Severe acute respiratory syndrome coronavirus 2
attacaaggtgtgctaccggc

SEQ ID NO: 139 - DNA, 21 bp, Severe acute respiratory syndrome coronavirus 2
attcaaggctccctcagttgc

SEQ ID NO: 140 - DNA, 18 bp, Unknown
  source: /note=“Description of Unknown ALK2 sense sequence”
ggcctcattattctctct

SEQ ID NO: 141 - DNA, 18 bp, Unknown
  source: /note=“Description of Unknown ALK2 sense sequence”
gtgttcgcagtatgtctt

SEQ ID NO: 142 - DNA, 18 bp, Unknown
  source: /note=“Description of Unknown ALK2 sense sequence”
gcctgcctgctgggagtt

SEQ ID NO: 143 - DNA, 21 bp, Unknown
  source: /note=“Description of Unknown SOD1 sense sequence”
gaaggaaagtaatggaccagt

SEQ ID NO: 144 - DNA, 21 bp, Unknown
  source: /note=“Description of Unknown SOD1 sense sequence”
ggtcctcactttaatcctcta

SEQ ID NO: 145 - DNA, 21 bp, Unknown
  source: /note=“Description of Unknown SOD1 sense sequence”
ggagacttgggcaatgtgact

SEQ ID NO: 146 - DNA, 18 bp, Unknown
  source: /note=“Description of Unknown ALK2 antisense sequence”
agagagaataatgaggcc

SEQ ID NO: 147 - DNA, 18 bp, Unknown
  source: /note=“Description of Unknown ALK2 antisense sequence”
aagacatactgcgaacac

SEQ ID NO: 148 - DNA, 18 bp, Unknown
  source: /note=“Description of Unknown ALK2 antisense sequence”
aactcccagcaggcaggc

SEQ ID NO: 149 - DNA, 21 bp, Unknown
  source: /note=“Description of Unknown SOD1 antisense sequence”
actggtccattactttccttc

SEQ ID NO: 150 - DNA, 21 bp, Unknown
  source: /note=“Description of Unknown SOD1 antisense sequence”
tagaggattaaagtgaggacc

SEQ ID NO: 151 - DNA, 21 bp, Unknown
  source: /note=“Description of Unknown SOD1 antisense sequence”
agtcacattgcccaagtctcc

SEQ ID NO: 152 - DNA, 741 bp, Artificial Sequence
  source: /note=“Description of Artificial Sequence Synthetic polynucleotide”
atagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccct

SEQ ID NO: 153 - DNA, 741 bp, Artificial Sequence
  source: /note=“Description of Artificial Sequence Synthetic polynucleotide”
gccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccct

SEQ ID NO: 154 - DNA, 617 bp, Artificial Sequence
  source: /note=“Description of Artificial Sequence Synthetic polynucleotide”
gccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaatagtgagtcgtattaacgtaccaacaaggcctcattattctctctacttgagagagaataatgaggcctttatcttagaggcatatccctacgtaccaacaagtgttcgcagtatgtcttacttgaagacatactgcgaacactttatcttagaggcatatccctacgtaccaacaagcctgcctgctgggagttacttgaactcccagcaggcaggctttatcttagaggcatatcccttttatcttagaggcatatccct

SEQ ID NO: 155 - DNA, 644 bp, Artificial Sequence
  source: /note=“Description of Artificial Sequence Synthetic polynucleotide”
atagtgagtcgtattaacgtaccaacaagaaggaaagtaatggaccagtacttgactggtccattactttccttctttatcttagaggcatatccctacgtaccaacaaggtcctcactttaatcctctaacttgtagaggattaaagtgaggacctttatcttagaggcatatccctacgtaccaacaaggagacttgggcaatgtgactacttgagtcacattgcccaagtctcctttatcttagaggcatatccctgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaatttatcttagaggcatatccct

SEQ ID NO: 156 - DNA, 869 bp, Artificial Sequence
  source: /note=“Description of Artificial Sequence Synthetic polynucleotide”
atagtgagtcgtattaacgtaccaacaagaaggaaagtaatggaccagtacttgactggtccattactttccttctttatcttagaggcatatccctacgtaccaacaaggtcctcactttaatcctctaacttgtagaggattaaagtgaggacctttatcttagaggcatatccctacgtaccaacaaggagacttgggcaatgtgactacttgagtcacattgcccaagtctcctttatcttagaggcatatccctgccaccatgggagtgcatgaatgtcctgcttggctgtggctgctgctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataatttatcttagaggcatatccct

SEQ ID NO: 157 - DNA, 638 bp, Artificial Sequence
  source: /note=“Description of Artificial Sequence Synthetic polynucleotide”
atagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaatttatcttagaggcatatccct

SEQ ID NO: 158 - DNA, 638 bp, Artificial Sequence
  source: /note=“Description of Artificial Sequence Synthetic polynucleotide”
gccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatcccttttatcttagaggcatatccct

SEQ ID NO: 159 - DNA, 4851 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtctagcagctcttggctgctgctgtctctggtggctgtgacagccgctcagagcaccattgaggaacaggccaagaccttcctggacaagttcaaccacgaggccgaggacctgttctaccagtctagcctggccagctggaactacaacaccaacatcaccgaagagaacgtgcagaacatgaacaacgccggcgacaagtggagcgccttcctgaaagagcagagcacactggcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgccatggtggatcaggcttgggatgcccagcggatcttcaaagaggccgagaagttcttcgtgtccgtgggcctgcctaatatgacccaaggcttctgggagaactccatgctgacagaccccggcaatgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccacgaagccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaatatgaacgtgcggccactgctgaactacttcgagcccctgttcacctggctgaaggaccagaacaagaacagcttcgtcggctggtccaccgattggagcccttacgccgaccagagcatcaaagtgcggatcagcctgaaaagcgccctgggcgataaggcctatgagtggaacgacaatgagatgtacctgttccggtccagcgtggcctatgctatgcggcagtactttctgaaagtcaagaaccagatgatcctgttcggcgaagaggatgtgcgcgtggccaacctgaagcctcggatcagcttcaacttcttcgtgactgcccctaagaacgtgtccgacatcatccccagaaccgaggtggaaaaggccatcagaatgagcagaagccggatcaacgacgccttccggctgaacgacaactccctggaattcctgggcattcagcccacactgggccctccaaatcagcctcctgtgtcctaaatagtgagtcgtattaacgtaccaacaagtgtgaccgaaaggtaagatgacttgcatcttacctttcggtcacactttatcttagaggcatatccctacgtaccaacaagaggtgatgaagtcagacaaaacttgtttgtctgacttcatcacctctttatcttagaggcatatccctacgtaccaacaagcaactgagggagccttgaatacttgattcaaggctccctcagttgctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 160 - DNA, 3082 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatccctgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 161 - DNA, 3082 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggactgacatctcaactgctgcctccactgttctttctgctggcctgcgccggcaattttgtgcacggccacaagtgcgacatcaccctgcaagagatcatcaagaccctgaacagcctgaccgagcagaaaaccctgtgcaccgagctgaccgtgaccgatatctttgccgccagcaagaacacaaccgagaaagagacattctgcagagccgccaccgtgctgagacagttctacagccaccacgagaaggacaccagatgcctgggagctacagcccagcagttccacagacacaagcagctgatccggttcctgaagcggctggacagaaatctgtggggactcgccggcctgaatagctgccctgtgaaagaggccaaccagtctaccctggaaaacttcctggaacggctgaaaaccatcatgcgcgagaagtacagcaagtgcagcagctgaatagtgagtcgtattaacgtaccaacaaggcgtggagctgagagataaacttgttatctctcagctccacgcctttatcttagaggcatatccctacgtaccaacaagggcctgtacctcatctactacttgagtagatgaggtacaggccctttatcttagaggcatatccctacgtaccaacaaggtatgagcccatctatctacttgagatagatgggctcatacctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 162 - DNA, 2958 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgaccatcctgtttctgacaatggtcatcagctacttcggctgcatgaaggccgtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgcttcagaagctgcgacctgcggcggctggaaatgtattgtgcccctctgaagcctgccaagagcgcctaaatagtgagtcgtattaacgtaccaacaaggcctcattattctctctacttgagagagaataatgaggcctttatcttagaggcatatccctacgtaccaacaagtgttcgcagtatgtcttacttgaagacatactgcgaacactttatcttagaggcatatccctacgtaccaacaagcctgcctgctgggagttacttgaactcccagcaggcaggctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 163 - DNA, 2985 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaagaaggaaagtaatggaccagtacttgactggtccattactttccttctttatcttagaggcatatccctacgtaccaacaaggtcctcactttaatcctctaacttgtagaggattaaagtgaggacctttatcttagaggcatatccctacgtaccaacaaggagacttgggcaatgtgactacttgagtcacattgcccaagtctcctttatcttagaggcatatccctgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 164 - DNA, 3210 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatatagtgagtcgtattaacgtaccaacaagaaggaaagtaatggaccagtacttgactggtccattactttccttctttatcttagaggcatatccctacgtaccaacaaggtcctcactttaatcctctaacttgtagaggattaaagtgaggacctttatcttagaggcatatccctacgtaccaacaaggagacttgggcaatgtgactacttgagtcacattgcccaagtctcctttatcttagaggcatatccctgccaccatgggagtgcatgaatgtcctgcttggctgtggctgctgctgagcctgctgtctctgcctctgggactgcctgttcttggagcccctcctagactgatctgcgacagcagagtgctggaaagatacctgctggaagccaaagaggccgagaacatcaccacaggctgtgccgagcactgcagcctgaacgagaatatcaccgtgcctgacaccaaagtgaacttctacgcctggaagcggatggaagtgggccagcaggctgtggaagtttggcaaggactggccctgctgagcgaagctgttctgagaggacaggctctgctggtcaacagctctcagccttgggaacctctgcaactgcacgtggacaaggccgtgtctggcctgagaagcctgaccacactgctgagagcactgggagcccagaaagaggccatctctccacctgatgctgcctctgctgcccctctgagaaccatcaccgccgacaccttcagaaagctgttccgggtgtacagcaacttcctgcggggcaagctgaagctgtacacaggcgaggcttgcagaaccggcgacagataatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 165 - DNA, 2980 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataatagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatccctgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaatttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 166 - DNA, 2979 bp, Artificial Sequence
  source: /note=“Description of Artificial Sequence Synthetic polynucleotide”
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgggcaagattagcagcctgcctacacagctgttcaagtgctgcttctgcgacttcctgaaagtgaagatgcacaccatgagcagcagccacctgttctatctggccctgtgcctgctgacctttaccagctctgctaccgccggacctgagacactttgtggcgctgaactggtggacgccctgcagtttgtgtgtggcgacagaggcttctacttcaacaagcccacaggctacggcagcagctctagaagggctcctcagaccggaatcgtggacgagtgctgtttcagaagctgcgacctgcggcggctggaaatgtattgtgcccctctgaagcctgccaagagcgcctaaatagtgagtcgtattaacgtaccaacaagaaagatgataagcccactctacttgagagtgggcttatcatctttctttatcttagaggcatatccctacgtaccaacaaggtgatgtctggtccatatgaacttgtcatatggaccagacatcacctttatcttagaggcatatccctacgtaccaacaagatgataagcccactctaacttgtagagtgggcttatcatctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 167 - DNA, 615 bp, Homo sapiens
atcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaa

SEQ ID NO: 168 - protein, 153 aa, Homo sapiens [sequence omitted]

SEQ ID NO: 169 - DNA, 1662 bp, Homo sapiens
cctttcccagatagcacgctccgccagtcccaagggtgcgcaaccggctgcactcccctcccgcgacccagggcccgggagcagcccccatgacccacacgcacgtctgcagcagccccgctcacgccccggcgagcctcaacccaggcgtcctgcccctgctctgaccccgggtggcccctacccctggcgacccctcacgcacacagcctctcccccacccccacccgcgcacgcacacatgcagataacagccccgacccccggccagagccgcagagtccctgggccaccccggccgctcgctgcgctgcgccgcaccgcgctgtcctcccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggataacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaa

SEQ ID NO: 170 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 171 - DNA, 22 bp, Artificial Sequence
  source: /note=“Description of Artificial Sequence Synthetic primer”
gacgtggagtatggcactatcg

SEQ ID NO: 172 - DNA, 23 bp, Artificial Sequence
  source: /note=“Description of Artificial Sequence Synthetic primer”
cactccaacagtgtaatctggcg

SEQ ID NO: 173 - DNA, 22 bp, Artificial Sequence
  source: /note=“Description of Artificial Sequence Synthetic primer”
acccgttgaaccccattcgtga

SEQ ID NO: 174 - DNA, 23 bp, Artificial Sequence
  source: /note=“Description of Artificial Sequence Synthetic primer”
gcctcactaaaccatccaatcgg

SEQ ID NO: 175 - DNA, 22 bp, Artificial Sequence
  source: /note=“Description of Artificial Sequence Synthetic primer”
ctcactctcaggagaccattgc

SEQ ID NO: 176 - DNA, 22 bp, Artificial Sequence
  source: /note=“Description of Artificial Sequence Synthetic primer”
ccacaagccaaacgacttccag

SEQ ID NO: 177 - RNA, 467 nt, Artificial Sequence
  source: /note=“Description of Artificial Sequence Synthetic polynucleotide”
auagugagucguauuaacguaccaacaacaaggaagugcuaaagaaacuuguucuuuagcacuuccuuguuuaucuuagaggcauaucccugccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaauuuaucuuagaggcauaucccu

SEQ ID NO: 178 - RNA, 465 nt, Artificial Sequence
  source: /note=“Description of Artificial Sequence Synthetic polynucleotide”
auagugagucguauuaacguaccaacaacaaggagugcuaaagaaacuuguucuuuagcacuccuuguuuaucuuagaggcauaucccugccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaauuuaucuuagaggcauaucccu

SEQ ID NO: 179 - RNA, 615 nt, Artificial Sequence
  source: /note=“Description of Artificial Sequence Synthetic polynucleotide”
auagugagucguauuaacguaccaacaacaaggagugcuaaagaaacuuguucuuuagcacuccuuguuuaucuuagaggcauaucccuacguaccaacaagagagugauugagaguggacuugccacucucaaucacucucuuuaucuuagaggcauaucccuacguaccaacaagagagcucugucuggaccacuuggguccagacagagcucucuuuaucuuagaggcauaucccugccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaauuuaucuuagaggcauaucccu

SEQ ID NO: 180 - RNA, 473 nt, Artificial Sequence
  source: /note=“Description of Artificial Sequence Synthetic polynucleotide”
auagugagucguauuaacguaccaacaagaaagaugauaagcccacucuacuugagagugggcuuaucaucuuucuuuaucuuagaggcauaucccugccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaauuuaucuuagaggcauaucccu

SEQ ID NO: 181 - RNA, 629 nt, Artificial Sequence
  source: /note=“Description of Artificial Sequence Synthetic polynucleotide”
auagugagucguauuaacguaccaacaagaaagaugauaagcccacucuacuugagagugggcuuaucaucuuucuuuaucuuagaggcauaucccuacguaccaacaaggugaugucugguccauaugaacuugucauauggaccagacaucaccuuuaucuuagaggcauaucccuacguaccaacaagaugauaagcccacucuaacuuguagagugggcuuaucaucuuuaucuuagaggcauaucccugccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaauuuaucuuagaggcauaucccu

SEQ ID NO: 182 - RNA, 978 nt, Artificial Sequence
  source: /note=“Description of Artificial Sequence Synthetic polynucleotide”
auagugagucguauuaacguaccaacaaggcguggagcugagagauaaacuuguuaucucucagcuccacgccuuuaucuuagaggcauaucccuacguaccaacaagggccuguaccucaucuacuacuugaguagaugagguacaggcccuuuaucuuagaggcauaucccuacguaccaacaagguaugagcccaucuaucuacuugagauagaugggcucauaccuuuaucuuagaggcauaucccuacguaccaacaagcaaugaggacccugagagauacuugaucucucaggguccucauugcuuuaucuuagaggcauaucccuacguaccaacaagcugaugggaacguggacuaacuuguaguccacguucccaucagcuuuaucuuagaggcauaucccuacguaccaacaagguccucagauuacuacaaacuuguuguaguaaucugaggaccuuuaucuuagaggcauaucccugccaccaugggacugacaucucaacugcugccuccacuguucuuucugcuggccugcgccggcaauuuugugcacggccacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacagccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcugauuuaucuuagaggcauaucccu

SEQ ID NO: 183 - RNA, 741 nt, Artificial Sequence
  source: /note=“Description of Artificial Sequence Synthetic polynucleotide”
auagugagucguauuaacguaccaacaaggcguggagcugagagauaaacuuguuaucucucagcuccacgccuuuaucuuagaggcauaucccuacguaccaacaagggccuguaccucaucuacuacuugaguagaugagguacaggcccuuuaucuuagaggcauaucccuacguaccaacaagguaugagcccaucuaucuacuugagauagaugggcucauaccuuuaucuuagaggcauaucccugccaccaugggacugacaucucaacugcugccuccacuguucuuucugcuggccugcgccggcaauuuugugcacggccacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacagccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcugauuuaucuuagaggcauaucccu

SEQ ID NO: 184 - RNA, 741 nt, Artificial Sequence
  source: /note=“Description of Artificial Sequence Synthetic polynucleotide”
gccaccaugggacugacaucucaacugcugccuccacuguucuuucugcuggccugcgccggcaauuuugugcacggccacaagugcgacaucacccugcaagagaucaucaagacccugaacagccugaccgagcagaaaacccugugcaccgagcugaccgugaccgauaucuuugccgccagcaagaacacaaccgagaaagagacauucugcagagccgccaccgugcugagacaguucuacagccaccacgagaaggacaccagaugccugggagcuacagcccagcaguuccacagacacaagcagcugauccgguuccugaagcggcuggacagaaaucuguggggacucgccggccugaauagcugcccugugaaagaggccaaccagucuacccuggaaaacuuccuggaacggcugaaaaccaucaugcgcgagaaguacagcaagugcagcagcugaauagugagucguauuaacguaccaacaaggcguggagcugagagauaaacuuguuaucucucagcuccacgccuuuaucuuagaggcauaucccuacguaccaacaagggccuguaccucaucuacuacuugaguagaugagguacaggcccuuuaucuuagaggcauaucccuacguaccaacaagguaugagcccaucuaucuacuugagauagaugggcucauaccuuuaucuuagaggcauaucccuuuuaucuuagaggcauaucccu

SEQ ID NO: 185 - RNA, 617 nt, Artificial Sequence
  source: /note=“Description of Artificial Sequence Synthetic polynucleotide”
gccaccaugaccauccuguuucugacaauggucaucagcuacuucggcugcaugaaggccgugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcugcuucagaagcugcgaccugcggcggcuggaaauguauugugccccucugaagccugccaagagcgccuaaauagugagucguauuaacguaccaacaaggccucauuauucucucuacuugagagagaauaaugaggccuuuaucuuagaggcauaucccuacguaccaacaaguguucgcaguaugucuuacuugaagacauacugcgaacacuuuaucuuagaggcauaucccuacguaccaacaagccugccugcugggaguuacuugaacucccagcaggcaggcuuuaucuuagaggcauaucccuuuuaucuuagaggcauaucccu

SEQ ID NO: 186 - RNA, 644 nt, Artificial Sequence
  source: /note=“Description of Artificial Sequence Synthetic polynucleotide”
auagugagucguauuaacguaccaacaagaaggaaaguaauggaccaguacuugacugguccauuacuuuccuucuuuaucuuagaggcauaucccuacguaccaacaagguccucacuuuaauccucuaacuuguagaggauuaaagugaggaccuuuaucuuagaggcauaucccuacguaccaacaaggagacuugggcaaugugacuacuugagucacauugcccaagucuccuuuaucuuagaggcauaucccugccaccaugggcaagauuagcagccugccuacacagcuguucaagugcugcuucugcgacuuccugaaagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcuguuucagaagcugcgaccugcggcggcuggaaauguauugugccccucugaagccugccaagagcgccuaauuuaucuuagaggcauaucccu

SEQ ID NO: 187 - RNA, 869 nt, Artificial Sequence
  source: /note=“Description of Artificial Sequence Synthetic polynucleotide”
auagugagucguauuaacguaccaacaagaaggaaaguaauggaccaguacuugacugguccauuacuuuccuucuuuaucuuagaggcauaucccuacguaccaacaagguccucacuuuaauccucuaacuuguagaggauuaaagugaggaccuuuaucuuagaggcauaucccuacguaccaacaaggagacuugggcaaugugacuacuugagucacauugcccaagucuccuuuaucuuagaggcauaucccugccaccaugggagugcaugaauguccugcuuggcuguggcugcugcugagccugcugucucugccucugggacugccuguucuuggagccccuccuagacugaucugcgacagcagagugcuggaaagauaccugcuggaagccaaagaggccgagaacaucaccacaggcugugccgagcacugcagccugaacgagaauaucaccgugccugacaccaaagugaacuucuacgccuggaagcggauggaagugggccagcaggcuguggaaguuuggcaaggacuggcccugcugagcgaagcuguucugagaggacaggcucugcuggucaacagcucucagccuugggaaccucugcaacugcacguggacaaggccgugucuggccugagaagccugaccacacugcugagagcacugggagcccagaaagaggccaucucuccaccugaugcugccucugcugccccucugagaaccaucaccgccgacaccuucagaaagcuguuccggguguacagcaacuuccugcggggcaagcugaagcuguacacaggcgaggcuugcagaaccggcgacagauaauuuaucuuagaggcauaucccu

SEQ ID NO: 188 - RNA, 638 nt, Artificial Sequence
  source: /note=“Description of Artificial Sequence Synthetic polynucleotide”
auagugagucguauuaacguaccaacaagaaagaugauaagcccacucuacuugagagugggcuuaucaucuuucuuuaucuuagaggcauaucccuacguaccaacaaggugaugucugguccauaugaacuugucauauggaccagacaucaccuuuaucuuagaggcauaucccuacguaccaacaagaugauaagcccacucuaacuuguagagugggcuuaucaucuuuaucuuagaggcauaucccugccaccaugggcaagauuagcagccugccuacacagcuguucaagugcugcuucugcgacuuccugaaagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcuguuucagaagcugcgaccugcggcggcuggaaauguauugugccccucugaagccugccaagagcgccuaauuuaucuuagaggcauaucccu

SEQ ID NO: 189 - RNA, 638 nt, Artificial Sequence
  source: /note=“Description of Artificial Sequence Synthetic polynucleotide”
gccaccaugggcaagauuagcagccugccuacacagcuguucaagugcugcuucugcgacuuccugaaagugaagaugcacaccaugagcagcagccaccuguucuaucuggcccugugccugcugaccuuuaccagcucugcuaccgccggaccugagacacuuuguggcgcugaacugguggacgcccugcaguuuguguguggcgacagaggcuucuacuucaacaagcccacaggcuacggcagcagcucuagaagggcuccucagaccggaaucguggacgagugcuguuucagaagcugcgaccugcggcggcuggaaauguauugugccccucugaagccugccaagagcgccuaaauagugagucguauuaacguaccaacaagaaagaugauaagcccacucuacuugagagugggcuuaucaucuuucuuuaucuuagaggcauaucccuacguaccaacaaggugaugucugguccauaugaacuugucauauggaccagacaucaccuuuaucuuagaggcauaucccuacguaccaacaagaugauaagcccacucuaacuuguagagugggcuuaucaucuuuaucuuagaggcauaucccuuuuaucuuagaggcauaucccu

SEQ ID NO: 190 - RNA, 2510 nt, Artificial Sequence
  source: /note=“Description of Artificial Sequence Synthetic polynucleotide”
gccaccaugucuagcagcucuuggcugcugcugucucugguggcugugacagccgcucagagcaccauugaggaacaggccaagaccuuccuggacaaguucaaccacgaggccgaggaccuguucuaccagucuagccuggccagcuggaacuacaacaccaacaucaccgaagagaacgugcagaacaugaacaacgccggcgacaaguggagcgccuuccugaaagagcagagcacacuggcccagauguacccucugcaagagauccagaaccugaccgugaagcuccagcugcaggcccuccagcagaauggaagcucugugcugagcgaggacaagagcaagcggcugaacaccauccugaauaccaugagcaccaucuacagcaccggcaaagugugcaaccccgacaauccccaagagugccugcugcuggaacccggccugaaugagaucauggccaacagccuggacuacaacgagagacugugggccugggagucuuggagaagcgaagugggaaagcagcugcggccccuguacgaggaauacguggugcugaagaacgagauggccagagccaaccacuacgaggacuacggcgacuauuggagaggcgacuacgaagugaauggcguggacggcuacgacuacagcagaggccagcugaucgaggacguggaacacaccuucgaggaaaucaagccucuguacgagcaucugcacgccuacgugcgggccaagcugaugaaugcuuaccccagcuacaucagccccaucggcugucugccugcucaucugcugggagacauguggggcagauucuggaccaaccuguacagccugacagugcccuucggccagaaaccuaacaucgacgugaccgacgccaugguggaucaggcuugggaugcccagcggaucuucaaagaggccgagaaguucuucguguccgugggccugccuaauaugacccaaggcuucugggagaacuccaugcugacagaccccggcaaugugcagaaagccgugugucauccuaccgccugggaucucggcaagggcgacuucagaauccugaugugcaccaaagugacgauggacgacuuccugacagcccaccacgagaugggccacauccaguacgauauggccuacgccgcucagcccuuccugcugagaaauggcgccaaugagggcuuccacgaagccgugggagagaucaugagccugucugccgccacaccuaagcaccugaagucuaucggacugcugagccccgacuuccaagaggacaacgagacagagaucaacuuccugcucaagcaggcccugaccaucgugggcacacugcccuuuaccuacaugcuggaaaaguggcgguggauggucuuuaagggcgagauccccaaggaccaguggaugaagaaauggugggagaugaagcgcgagaucgugggcguuguggaaccugugccucacgacgagacauacugcgauccugccagccuguuucacguguccaacgacuacuccuucauccgguacuacacccggacacuguaccaguuccaguuucaagaggcucugugccaggccgccaagcacgaaggaccucugcacaagugcgacaucagcaacucuacagaggccggacagaaacuguucaacaugcugcggcugggcaagagcgagccuuggacacuggcucuggaaaaugucgugggcgccaagaauaugaacgugcggccacugcugaacuacuucgagccccuguucaccuggcugaaggaccagaacaagaacagcuucgucggcugguccaccgauuggagcccuuacgccgaccagagcaucaaagugcggaucagccugaaaagcgcccugggcgauaaggccuaugaguggaacgacaaugagauguaccuguuccgguccagcguggccuaugcuaugcggcaguacuuucugaaagucaagaaccagaugauccuguucggcgaagaggaugugcgcguggccaaccugaagccucggaucagcuucaacuucuucgugacugccccuaagaacguguccgacaucauccccagaaccgagguggaaaaggccaucagaaugagcagaagccggaucaacgacgccuuccggcugaacgacaacucccuggaauuccugggcauucagcccacacugggcccuccaaaucagccuccuguguccuaaauagugagucguauuaacguaccaacaagugugaccgaaagguaagaugacuugcaucuuaccuuucggucacacuuuaucuuagaggcauaucccuacguaccaacaagaggugaugaagucagacaaaacuuguuugucugacuucaucaccucuuuaucuuagaggcauaucccuacguaccaacaagcaacugagggagccuugaauacuugauucaaggcucccucaguugcuuuaucuuagaggcauaucccuuuuaucuuagaggcauaucccu

SEQ ID NO: 191 - RNA, 220 nt, Artificial Sequence
  source: /note=“Description of Artificial Sequence Synthetic polynucleotide”
  misc_feature (1)..(220): /note=“This sequence may encompass 1-220 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 192 - RNA, 220 nt, Artificial Sequence; identical to SEQ ID NO: 191
  source: /note=“Description of Artificial Sequence Synthetic polynucleotide”
  misc_feature (1)..(220): /note=“This sequence may encompass 1, 3, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215,”
  misc_feature (1)..(220): /note=“CONT. FROM ABOVE or 220 nucleotides”

SEQ ID NO: 193 - RNA, 120 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 194 - RNA, 220 nt, Artificial Sequence; identical to SEQ ID NO: 191
  source: /note=“Description of Artificial Sequence Synthetic polynucleotide”
  misc_feature (1)..(220): /note=“This sequence may encompass 1 to 20, 1 to 40, 1 to 60, 1 to 80, 1 to 100, 1 to 120, 1 to 140, 1 to 160, 1 to 180, 1 to 200, 1 to 220, 20 to 40, 20 to 60, 20 to 80, 20 to 100, 20 to 120, 20 to 140, 20 to 160, 20 to 180, 20 to 200, 20 to”
  misc_feature (1)..(220): /note=“CONT. FROM ABOVE 220, 40 to 60, 40 to 80, 40 to 100, 40 to 120, 40 to 140, 40 to 160, 40 to 180, 40 to 200, 40 to 220, 60 to 80, 60 to 100, 60 to 120, 60 to 140, 60 to 160, 60 to 180, 60 to 200, 60 to 220, 80 to 100, 80 to 120, 80 to 140,”
  misc_feature (1)..(220): /note=“CONT. FROM ABOVE 80 to 160, 80 to 180, 80 to 200, 80 to 220, 100 to 120, 100 to 140, 100 to 160, 100 to 180, 100 to 200, 100 to 220, 120 to 140, 120 to 160, 120 to 180, 120 to 200, 120 to 220, 140 to 160, 140 to 180, 140 to 200, 140”
  misc_feature (1)..(220): /note=“CONT. FROM ABOVE to 220, 160 to 180, 160 to 200, 160 to 220, 180 to 200, 180 to 220, or 200 to 220 nucleotides”

SEQ ID NO: 195 - RNA, 220 nt, Artificial Sequence; identical to SEQ ID NO: 191
  source: /note=“Description of Artificial Sequence Synthetic polynucleotide”
  misc_feature (1)..(220): /note=“This sequence may encompass 1, 20, 40, 60, 80, 100, 120, 140, 160, 180, 200, or 220 nucleotides”

SEQ ID NO: 196 - RNA, 220 nt, Artificial Sequence; identical to SEQ ID NO: 191
  source: /note=“Description of Artificial Sequence Synthetic polynucleotide”
  misc_feature (1)..(220): /note=“This sequence may encompass 20, 40, 60, 80, 100, 120, 140, 160, 180, 200, or 220 nucleotides”

SEQ ID NO: 197 - DNA, 120 bp, Artificial Sequence
  source: /note=“Description of Artificial Sequence Synthetic polynucleotide”
tttttttttttttttttttttttttttttttttttttttttttttttttttttttttttttttttttttttttttttttttttttttttttttttttttttttttttttttttttttttt

SEQ ID NO: 198 - protein, 10 aa, Artificial Sequence
  source: /note=“Description of Artificial Sequence Synthetic peptide”
VKMHTMSSSH

SEQ ID NO: 199 - RNA, 200 nt, Artificial Sequence
  source: /note=“Description of Artificial Sequence Synthetic polynucleotide”
  misc_feature (1)..(200): /note=“This sequence may encompass 1, 20, 40, 60, 80, 100, 120, 140, 160, 180, or 200 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2022/0389423 A1
(43) Pub. Date: Dec. 8, 2022

(54) Compositions and Methods for Simultaneously Modulating Expression of Genes

(71) Applicant: Versameb AG, Basel (CH)

(72) Inventors: Justin Antony SELVARAJ, Weil am Rhein (DE); Friedrich Metzger, Freiburg (DE); Hervé Schaffhauser, Habsheim (FR); Petra HILLMANN-WÜLLNER, Zurich (CH)

(21) Appl. No.: 17/846,288

(22) Filed: Jun. 22, 2022

Related U.S. Application Data
(63) Continuation of application No. PCT/IB2020/001091, filed on Dec. 21, 2020.
(60) Provisional application No. 63/042,890, filed on Jun. 23, 2020.

(30) Foreign Application Priority Data
Dec. 23, 2019 (EP) EP19219276.3

(51) Int. Cl. C12N 15/113; A61K 48/00; C07K 14/52; C07K 14/54; C07K 14/565; C07K 14/65; A61K 31/713

(52) U.S. Cl. CPC C12N 15/113; A61K 48/005; C07K 14/52; C07K 14/5406; C07K 14/565; C07K 14/65; C12N 15/1136; A61K 31/713; C12N 15/1137; A61K 38/00

(57) ABSTRACT

The present invention relates to compositions of recombinant polynucleic acid constructs comprising at least one nucleic acid sequence encoding an siRNA capable of binding to a target mRNA and at least one nucleic acid sequence encoding a gene of interest. Also disclosed herein is use of the compositions in treating a disease or a condition and in simultaneously modulating expression of two or more genes.

### CROSS-REFERENCE TO RELATED APPLICATIONS

This application is a continuation of International Application No. PCT/IB2020/001091, filed Dec. 21, 2020, which claims the benefit of European Patent Application No. EP19219276.3, filed Dec. 23, 2019 and U.S. Provisional Application No. 63/042,890, filed Jun. 23, 2020, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 18, 2020, is named 57623_701_601_SL.txt and is 326,570 bytes in size.

### BACKGROUND

Numerous human diseases and disorders are caused by combinations of higher and/or lower expression levels of certain proteins compared to the expression levels of these proteins in humans without the disease or disorder. Combinatorial therapies to increase the expression and/or secretion of a target protein and to decrease the expression of another, different target protein, may have a therapeutic effect. For example, therapies for coronavirus infection, e.g., COVID-19, the disease caused by infection with the coronavirus SARS-CoV-2, that effectively and specifically decrease production of one or more target gene products and concomitantly increase production of others are needed.

### SUMMARY

The present invention relates to modulating expression of two or more proteins or nucleic acid sequences simultaneously using one recombinant polynucleic acid or RNA construct. In some embodiments, the recombinant polynucleic acid or RNA construct of the present invention simultaneously upregulate and downregulate the expression of two or more proteins or nucleic acid sequences by providing a nucleic acid sequence encoding a single or multiple small interfering RNA (siRNA) capable of binding to specific targets and a nucleic acid sequence encoding single or multiple proteins for overexpression. In some embodiments, the present invention is useful to treat diseases and disorders wherein a specific physiological mechanism (e.g., catabolism) can be controlled by siRNA while another physiological mechanism can be activated (e.g., anabolism) by overexpression of a therapeutic protein in parallel.

The invention also provides a recombinant polynucleic acid or RNA construct that comprises a polynucleic acid or RNA that encodes or comprises: one or more small interfering RNAs (siRNAs) that are capable of binding to one or more coronavirus target RNAs and/or one or more RNAs encoding a host protein, e.g., a viral entry element or a proinflammatory cytokine; and a nucleic acid sequence that encodes one or more proteins for overexpression, e.g., a host anti-inflammatory cytokine or a decoy protein, e.g., a soluble Angiotensin Converting Enzyme-2 (ACE2). In some embodiments, the coronavirus target RNA is an mRNA encoding one or more coronavirus proteins, or a noncoding RNA. The present invention thus provides embodiments wherein a single polynucleotide molecule both inhibit a virus and modulate the host inflammatory response.

In some aspects, provided herein, is a composition comprising a recombinant polynucleic acid construct comprising: (i) at least one nucleic acid sequence encoding or comprising a small interfering RNA (siRNA) capable of binding to a target RNA (e.g., mRNA); and (ii) at least one nucleic acid sequence encoding a gene of interest; wherein the target RNA is different from an mRNA encoded by the gene of interest. In some embodiments, the target RNA is an mRNA.

In some embodiments, (i) and (ii) are comprised in 5′ to 3′ direction. In some embodiments, (i) and (ii) are not comprised in 5′ to 3′ direction. In some embodiments, the recombinant polynucleic acid construct further comprises a nucleic acid sequence encoding or comprising a linker. In some embodiments, the nucleic acid sequence encoding or comprising the linker connects (i) and (ii). In some embodiments, the linker comprises a tRNA linker. In some aspects, the nucleic acid sequence encoding or comprising the linker is at least 6 nucleic acid residues in length. In some aspects, the nucleic acid sequence encoding or comprising the linker is up to 80 nucleic acid residues in length. In some aspects, the nucleic acid sequence encoding or comprising the linker is about 6 to about 80 nucleic acid residues in length. In some aspects, the nucleic acid sequence encoding or comprising the linker is about 6 to about 15 nucleic acid residues in length.

In some embodiments, the recombinant polynucleic acid construct is circular. In some embodiments, the recombinant polynucleic acid construct is linear. In some embodiments, the recombinant polynucleic acid construct is DNA. In some embodiments, the recombinant polynucleic acid construct is RNA.

In some embodiments, the recombinant polynucleic acid construct further comprises a nucleic acid sequence encoding or comprising a poly(A) tail. In some embodiments, the poly(A) tail comprises 1-220 base pairs of poly(A) (SEQ ID NO: 191). In some embodiments, the recombinant polynucleic acid construct further comprises a 5′ cap. In some embodiments, the 5′ cap comprises an anti-reverse CAP analog, Clean Cap, Cap 0, Cap 1, Cap 2, or Locked Nucleic Acid cap (LNA-cap). In some embodiments, the 5′ cap comprises m₂^{7,3′-O}G(5′)ppp(5′)G, m7G, m7G(5′)G, m7GpppG, or m7GpppGm. In some embodiments, the recombinant polynucleic acid construct further comprises a promoter. In some embodiments, the promoter is selected from the group consisting of T3, T7, SP6, P60, Syn5, and KP34. In some embodiments, the promoter is a T7 promoter. In some embodiments, the T7 promoter is upstream of the at least one nucleic acid sequence encoding or comprising the siRNA. In some embodiments, the T7 promoter comprises a sequence comprising TAATACGACTCACTATA (SEQ ID NO: 25). In some embodiments, the recombinant polynucleic acid construct further comprises a Kozak sequence.

In some embodiments, the siRNA comprises 1-10 copies of siRNA. In some embodiments, the siRNA comprises a sense siRNA strand. In some embodiments, the siRNA comprises an anti-sense siRNA strand. In some embodiments, the siRNA comprises a sense and an anti-sense siRNA strand. In some embodiments, the siRNA does not affect the expression of the gene of interest. In some embodiments, the siRNA does not inhibit the expression of the gene of interest. In some embodiments, the recombinant polynucleic acid construct comprises two or more nucleic acid sequences encoding or comprising an siRNA capable of binding to a target mRNA. In some embodiments, the recombinant polynucleic acid construct further comprises a nucleic acid sequence encoding or comprising a linker. In some embodiments, the nucleic acid sequence encoding or comprising the linker connects each of the two or more nucleic acid sequences encoding or comprising the siRNA capable of binding to the target mRNA. In some embodiments, the linker comprises a tRNA linker. In some embodiments, each of the two or more nucleic acid sequences encodes or comprises an siRNA capable of binding to a same target mRNA. In some embodiments, each of the two or more nucleic acid sequences encodes or comprises an siRNA capable of binding to a different target mRNA. In some embodiments, each of at least two of the two or more nucleic acid sequences encodes or comprises an siRNA capable of binding to the same target mRNA or different target mRNAs.

In some embodiments, the target RNA is an mRNA. In some embodiments, the target mRNA encodes a protein selected from the group consisting of Tumor Necrosis Factor alpha (TNF-alpha), interleukin, Angiotensin Converting Enzyme-2 (ACE2), SARS CoV-2 ORF1ab, SARS CoV-2 S, and SARS CoV-2 N. In some embodiments, the target RNA is an mRNA encoding a protein selected from the group consisting of Tumor Necrosis Factor alpha (TNF-alpha), interleukin, Angiotensin Converting Enzyme-2 (ACE2), SARS CoV-2 ORF1ab, SARS CoV-2 S, SARS CoV-2 N, Superoxide dismutase-1 (SOD1), and Activin receptor-like kinase-2 (ALK2).

In some embodiments, the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Interleukin 6 (IL-6), Interleukin 6R (IL-6R), Interleukin 6R-alpha (IL-6R-alpha), Interleukin 6R-beta (IL-6R-beta), Angiotensin Converting Enzyme-2 (ACE2), SARS CoV-2 ORF1ab, SARS CoV-2 S, and SARS CoV-2 N. In some embodiments, the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Interleukin 6 (IL-6), Interleukin 6R (IL-6R), Interleukin 6R-alpha (IL-6R-alpha), Interleukin 6R-beta (IL-6R-beta), Angiotensin Converting Enzyme-2 (ACE2), SARS CoV-2 ORF1ab, SARS CoV-2 S, SARS CoV-2 N, Superoxide dismutase-1 (SOD1), and Activin receptor-like kinase-2 (ALK2).

In some embodiments, the target mRNA encodes a protein selected from the group consisting of Interleukin 8 (IL-8), Interleukin 1 beta (IL-1 beta), Interleukin 17 (IL-17), and Tumor Necrosis Factor alpha (TNF-alpha).

In some embodiments, the target RNA is a coronavirus target RNA or a coronavirus host cell target RNA. In some embodiments, the coronavirus target RNA is an mRNA that encodes a coronavirus protein. In some embodiments, the coronavirus target RNA is a coronavirus noncoding RNA. In some embodiments, the coronavirus protein is a Spike protein (S), a Nucleocapsid protein (N), a non-structural protein (NSP), or an ORF1ab (polyprotein PP1ab) protein, e.g., a SARS CoV-2 NSP1 protein. In some embodiments, the coronavirus target RNA is a SARS CoV-2 NSP12 and 13 coding RNA. In some embodiments, the coronavirus host cell target is a host cell protein. In some embodiments, the host cell is a human cell. In some embodiments, the host cell protein is ACE2, IL-6, IL-6R-alpha, or IL-6R-beta.

In some embodiments, the expression of the target RNA is modulated by the siRNA capable of binding to the target RNA. In some embodiments, the expression of the target RNA is downregulated by the siRNA capable of binding to the target RNA. In some embodiments, the expression of the target RNA is modulated by the siRNA capable of binding to the target mRNA. In some embodiments, the expression of the target RNA is downregulated by the siRNA capable of binding to the target RNA. In some embodiments, the expression of the target RNA is modulated by the siRNA capable of specifically binding to the target RNA. In some embodiments, the expression of the target RNA is downregulated by the siRNA capable of specifically binding to the target RNA.

In some embodiments, the recombinant nucleic acid construct comprises two or more nucleic acid sequences encoding a gene of interest. In some embodiments, each of the two or more nucleic acid sequences encodes a same gene of interest. In some embodiments, each of the two or more nucleic acid sequences encodes a different gene of interest. In some embodiments, each of the two or more nucleic acid sequences encoding the gene of interest comprises a nucleic acid sequence encoding a secretory protein. In some embodiments, each of the two or more nucleic acid sequences encoding the gene of interest comprises a nucleic acid sequence encoding an intracellular protein. In some embodiments, each of the two or more nucleic acid sequences encoding the gene of interest comprises a nucleic acid sequence encoding an intraorganelle protein. In some embodiments, each of the two or more nucleic acid sequences encoding the gene of interest comprises a nucleic acid sequence encoding a membrane protein. In some embodiments, the recombinant polynucleic acid construct further comprises a nucleic acid sequence encoding or comprising a linker. In some embodiments, the nucleic acid sequence encoding or comprising the linker connects each of the two or more nucleic acid sequences encoding the gene of interest. In some embodiments, the linker comprises a 2A peptide linker or a tRNA linker. In some embodiments, the gene of interest is selected from the group consisting of Insulin-like Growth Factor 1 (IGF-1), Interleukin 4 (IL-4), Interferon beta (IFN beta), Interferon alpha (IFN alpha), ACE2 soluble receptor, Interleukin 37 (IL-37), and Interleukin 38 (IL-38). In some embodiments, the gene of interest is selected from the group consisting of Insulin-like Growth Factor 1 (IGF-1), Interleukin 4 (IL-4), Interferon beta (IFN beta), and ACE2 soluble receptor. In some embodiments, the gene of interest is selected from the group consisting of Insulin-like Growth Factor 1 (IGF-1), Interleukin 4 (IL-4), Interferon beta (IFN beta), ACE2 soluble receptor, and Erythropoietin (EPO). In some embodiments, the gene of interest is selected from the group consisting of Insulin-like Growth Factor 1 (IGF-1), and Interleukin 4.

In some embodiments, the gene of interest encodes a coronavirus host protein. In some embodiments, the host protein encoded by the gene of interest is selected from: an IFN-α, e.g., interferon alpha-n3, interferon alpha-2a, or interferon alpha-2b, an IFN-β, an IFN-δ, an IFN-ε, an IFN-κ, an IFN-ν, an IFN-τ, an IFN-ω, an IFN-γ, an IFN-λ, IL-37, IL-38, and a soluble ACE2 receptor.

In some embodiments, the expression of the gene of interest is modulated by expressing an mRNA or a protein encoded by the gene of interest. In some embodiments, the expression of the gene of interest is upregulated by expressing an mRNA or a protein encoded by the gene of interest. In some embodiments, the recombinant polynucleic acid construct is codon-optimized. In some embodiments, the recombinant polynucleic acid construct is not codon-optimized.

In some embodiments, the recombinant polynucleic acid construct further comprises a nucleic acid sequence encoding a target motif. In some embodiments, the nucleic acid sequence encoding the target motif is operably linked to the at least one nucleic acid sequence encoding the gene of interest. In some embodiments, the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some embodiments, the target motif is selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid. In some embodiments, the signal peptide is selected from the group consisting of (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide is homologous to a protein encoded by the gene of interest, wherein the signal peptide is homologous to the protein encoded by the gene of interest is modified by insertion, deletion, and/or substitution of at least one amino acid; and (d) a naturally occurring amino acid sequence which does not have the function of a signal peptide in nature, wherein the naturally occurring amino acid sequence is optionally modified by insertion, deletion, and/or substitution of at least one amino acid. In some embodiments, the amino acids 1-9 of the N-terminal end of the signal peptide have an average hydrophobic score of above 2. In some embodiments, the recombinant polynucleic acid construct is a vector suitable for gene therapy. In some aspects, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some aspects, the at least one nucleic acid sequence encoding or comprising a small interfering RNA (siRNA) capable of binding to a target RNA (i) and the at least one nucleic acid sequence encoding a gene of interest (ii) are present in a sequential manner. In some aspects, the nucleic acid sequence encoding or comprising a small interfering RNA (siRNA) capable of binding to a target RNA (i) is upstream of the at least one nucleic acid sequence encoding a gene of interest (ii). In some aspects, the nucleic acid sequence encoding or comprising a small interfering RNA (siRNA) capable of binding to a target RNA (i) is downstream of the at least one nucleic acid sequence encoding a gene of interest (ii). In some aspects, the nucleic acid sequence encoding or comprising a small interfering RNA (siRNA) capable of binding to a target RNA (i) is upstream or downstream of the at least one nucleic acid sequence encoding a gene of interest (ii). In some aspects, the siRNA capable of binding to a target RNA binds to an exon of a target mRNA. In some aspects, the siRNA capable of binding to a target RNA specifically binds to one target RNA. In some aspects, the siRNA capable of binding to a target RNA is not encoded by or comprised of an intron sequence of the gene of interest. In some aspects, the gene of interest is expressed without RNA splicing.

In some aspects, provided herein, is a composition comprising a recombinant RNA construct comprising: (i) a small interfering RNA (siRNA) capable of binding to a target RNA (e.g., mRNA); and (ii) an mRNA encoding a gene of interest; wherein the target RNA is different from the mRNA encoding the gene of interest. In some embodiments, the target RNA is an mRNA.

In some embodiments, (i) and (ii) are comprised in 5′ to 3′ direction. In some embodiments, (i) and (ii) are not comprised in 5′ to 3′ direction. In some embodiments, the recombinant RNA construct further encodes or comprises a linker. In some embodiments, the nucleic acid sequence encoding or comprising the linker connects (i) and (ii). In some embodiments, the linker comprises a tRNA linker.

In some embodiments, the recombinant RNA construct further comprises a poly(A) tail. In some embodiments, the poly(A) tail comprises 1-220 base pairs of poly(A) (SEQ ID NO: 191). In some embodiments, the recombinant RNA construct further comprises a 5′ cap. In some embodiments, the 5′ cap comprises an anti-reverse CAP analog, Clean Cap, Cap 0, Cap 1, Cap 2, or Locked Nucleic Acid cap (LNA-cap). In some embodiments, the 5′ cap comprises m₂^{7,3′-O}G(5′)ppp(5′)G, m7G, m7G(5′)G, m7GpppG, or m7GpppGm. In some embodiments, the recombinant RNA construct further comprises a Kozak sequence.

In some embodiments, the siRNA comprises 1-10 copies of siRNA. In some embodiments, the siRNA comprises a sense siRNA strand. In some embodiments, the siRNA comprises an anti-sense siRNA strand. In some embodiments, the siRNA comprises a sense and an anti-sense siRNA strand. In some embodiments, the siRNA does not affect the expression of the gene of interest. In some embodiments, the siRNA does not inhibit the expression of the gene of interest. In some embodiments, the recombinant RNA construct comprises two or more nucleic acid sequences comprising an siRNA capable of binding to a target mRNA. In some embodiments, the recombinant RNA construct further comprises a linker. In some embodiments, the linker connects each of the two or more nucleic acid sequences comprising the siRNA capable of binding to the target mRNA. In some embodiments, the linker comprises a tRNA linker. In some embodiments, each of the two or more nucleic acid sequences comprises an siRNA capable of binding to a same target mRNA. In some embodiments, each of the two or more nucleic acid sequences comprises an siRNA capable of binding to a different target mRNA. In some embodiments, at least two of the two or more nucleic acid sequences encodes or comprises an siRNA capable of binding to the same or a different target mRNA.

In some embodiments, the target RNA is an mRNA encoding a protein selected from the group consisting of Tumor Necrosis Factor alpha (TNF-alpha), interleukin, Angiotensin Converting Enzyme-2 (ACE2), SARS CoV-2 ORF1ab, SARS CoV-2 S, and SARS CoV-2 N. In some embodiments, the target RNA is an mRNA encoding a protein selected from the group consisting of Tumor Necrosis Factor alpha (TNF-alpha), interleukin, Angiotensin Converting Enzyme-2 (ACE2), SARS CoV-2 ORF1ab, SARS CoV-2 S, SARS CoV-2 N, Superoxide dismutase-1 (SOD1), and Activin receptor-like kinase-2 (ALK2).

In some embodiments, the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Interleukin 6 (IL-6), Interleukin 6R (IL-6R), Interleukin 6R-alpha (IL-6R-alpha), Interleukin 6R-beta (IL-6R-beta), Angiotensin Converting Enzyme-2 (ACE2), SARS CoV-2 ORF1ab, SARS CoV-2 S, and SARS CoV-2 N. In some embodiments, the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Interleukin 6 (IL-6), Interleukin 6R (IL-6R), Interleukin 6R-alpha (IL-6R-alpha), Interleukin 6R-beta (IL-6R-beta), Angiotensin Converting Enzyme-2 (ACE2), SARS CoV-2 ORF1ab, SARS CoV-2 S, SARS CoV-2 N, Superoxide dismutase-1 (SOD1), and Activin receptor-like kinase-2 (ALK2).

In some embodiments, the target mRNA is selected from the group consisting of Interleukin 8 (IL-8), Interleukin 1 beta (IL-1 beta), Interleukin 17 (IL-17), and Tumor Necrosis Factor alpha (TNF-alpha).

In some embodiments, the target RNA is a coronavirus target RNA or a coronavirus host cell target RNA. In some embodiments, the coronavirus target RNA is an mRNA that encodes a coronavirus protein. In some embodiments, the coronavirus target RNA is a coronavirus noncoding RNA. In some embodiments, the coronavirus protein is a Spike protein (S), a Nucleocapsid protein (N), a non-structural protein (NSP), or an ORF1ab (polyprotein PP1ab) protein, e.g., a SARS CoV-2 NSP1 protein. In some embodiments, the coronavirus target RNA is a SARS CoV-2 NSP12 and 13 coding RNA. In some embodiments, the coronavirus host cell target is a host cell protein. In some embodiments, the host cell is a human cell. In some embodiments, the host cell protein is ACE2, IL-6, IL-6R-alpha, or IL-6R-beta.

In some embodiments, the expression of the target mRNA is modulated by the siRNA capable of binding to the target mRNA. In some embodiments, the expression of the target mRNA is downregulated by the siRNA capable of binding to the target mRNA.

In some embodiments, the recombinant RNA construct comprises two or more nucleic acid sequences encoding a gene of interest. In some embodiments, each of the two or more nucleic acid sequences encodes a same gene of interest. In some embodiments, each of the two or more nucleic acid sequences encodes a different gene of interest. In some embodiments, each of the two or more nucleic acid sequences encoding the gene of interest comprises a nucleic acid sequence encoding a secretory protein. In some embodiments, each of the two or more nucleic acid sequences encoding the gene of interest comprises a nucleic acid sequence encoding an intracellular protein. In some embodiments, each of the two or more nucleic acid sequences encoding the gene of interest comprises a nucleic acid sequence encoding an intraorganelle protein. In some embodiments, each of the two or more nucleic acid sequences encoding the gene of interest comprises a nucleic acid sequence encoding a membrane protein. In some embodiments, the recombinant RNA construct further comprises a linker or a nucleic acid sequence encoding a linker. In some embodiments, the nucleic acid sequence encoding or comprising the linker connects each of the two or more nucleic acid sequences encoding the gene of interest. In some embodiments, the linker comprises a 2A peptide linker, a tRNA linker or a flexible linker. In some embodiments, the gene of interest is selected from the group consisting of Insulin-like Growth Factor 1 (IGF-1), Interleukin 4 (IL-4), Interferon beta (IFN beta), Interferon alpha (IFN alpha), ACE2 soluble receptor, Interleukin 37 (IL-37), and Interleukin 38 (IL-38). In some embodiments, the gene of interest is selected from the group consisting of Insulin-like Growth Factor 1 (IGF-1), Interleukin 4 (IL-4), Interferon beta (IFN beta), and ACE2 soluble receptor. In some embodiments, the gene of interest is selected from the group consisting of Insulin-like Growth Factor 1 (IGF-1), Interleukin 4 (IL-4), Interferon beta (IFN beta), ACE2 soluble receptor, and Erythropoietin (EPO). In some embodiments, the gene of interest is selected from the group consisting of Insulin-like Growth Factor 1 (IGF-1), and IL-4.

In some embodiments, the gene of interest encodes a coronavirus host protein. In some embodiments, the host protein is selected from: an IFN-α, e.g., interferon alpha-n3, interferon alpha-2a, or interferon alpha-2b, an IFN-β, an IFN-δ, an IFN-ε, an IFN-κ, an IFN-ν, an IFN-τ, an IFN-ω, an IFN-γ, an IFN-λ, IL-37, IL-38, and a soluble ACE2 receptor.

In some embodiments, the expression of the gene of interest is modulated by expressing an mRNA or a protein encoded by the gene of interest. In some embodiments, the expression of the gene of interest is unregulated by expressing an mRNA or a protein encoded by the gene of interest. In some embodiments, the recombinant RNA construct is codon-optimized. In some embodiments, the recombinant RNA construct is not codon-optimized.

In some embodiments, the recombinant RNA construct further comprises a nucleic acid sequence encoding a target motif. In some embodiments, the nucleic acid sequence encoding the target motif is operably linked to the at least one nucleic acid sequence encoding the gene of interest. In some embodiments, the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some embodiments, the target motif is selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid.

In some embodiments, the signal peptide is selected from the group consisting of (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide is homologous to a protein encoded by the gene of interest, wherein the signal peptide is homologous to the protein encoded by the gene of interest is modified by insertion, deletion, and/or substitution of at least one amino acid; and (d) a naturally occurring amino acid sequence which does not have the function of a signal peptide in nature, wherein the naturally occurring amino acid sequence is optionally modified by insertion, deletion, and/or substitution of at least one amino acid. In some embodiments, the amino acids 1-9 of the N-terminal end of the signal peptide have an average hydrophobic score of above 2.

In some aspects, provided herein, is a cell comprising the composition of any recombinant polynucleic acid or RNA construct described herein. In some aspects, provided herein, is a pharmaceutical composition comprising the composition of any recombinant polynucleic acid or RNA construct described herein and a pharmaceutically acceptable excipient. In some aspects, provided herein, is a method of treating a disease or a condition in a subject in need thereof, comprising administering to the subject the pharmaceutical composition described herein. In some embodiments, the disease or the condition is selected from the group consisting of intervertebral disc disease (IVDD), osteoarthritis, and psoriasis. In some embodiments, the disease or the condition is selected from the group consisting of intervertebral disc disease (IVDD), osteoarthritis, psoriasis, fibrodysplasia ossificans progressiva (FOP) and Amyotrophic lateral sclerosis (ALS). In some embodiments, the disease or the condition is selected from the group consisting of intervertebral disc disease (IVDD), osteoarthritis, psoriasis, fibrodysplasia ossificans progressiva (FOP), amyotrophic lateral sclerosis (ALS), and a coronavirus infection, or a disease or condition resulting from or associated with a coronavirus infection. In some embodiments, the subject is a human.

In some aspects, provided herein, is a method of treating a disease or a condition in a subject in need thereof, comprising administering to the subject a pharmaceutical composition described herein. In some embodiments, the disease or condition in the subject is a coronavirus infection, or a disease or condition resulting from or associated with a coronavirus infection. In some embodiments, the coronavirus is SARS-CoV, MERS-CoV, or SARS-CoV-2. In some embodiments, the disease or disorder is SARS, MERS, or COVID-19.

In some aspects, provided herein, is a method of simultaneously expressing an siRNA and an mRNA from a single RNA transcript in a cell, comprising introducing into the cell the composition of any recombinant polynucleic acid or RNA construct described herein. In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest, and wherein the expression of the target mRNA and the gene of interest is modulated simultaneously.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest, and wherein the expression of the target mRNA is downregulated and the expression of the gene of interest is upregulated simultaneously. In some embodiments, the expression of the target mRNA is downregulated by the siRNA capable of binding to the target mRNA. In some embodiments, the expression of the gene of interest is upregulated by expressing an mRNA or a protein encoded by the gene of interest.

In some aspects, provided herein, is a method of producing an RNA construct comprising a small interfering RNA (siRNA) capable of binding to a target messenger RNA (mRNA), and an mRNA encoding a gene of interest, wherein the target mRNA is different from the mRNA encoding the gene of interest, the method comprising: (a) providing, for in vitro transcription reaction: (i) a polynucleic acid construct comprising a promoter, at least one nucleic acid sequence encoding an siRNA capable of binding to a target mRNA, at least one nucleic acid sequence encoding a gene of interest, and a nucleic acid sequence encoding poly(A) tail; (ii) an RNA polymerase; and (iii) a mixture of nucleotide triphosphates (NTPs); and (b) isolating and purifying transcribed RNAs from the in vitro transcription reaction mixture, thus producing the RNA construct. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase. In some embodiments, the RNA polymerase is T7 RNA polymerase. In some embodiments, the mixture of NTPs comprises unmodified NTPs. In some embodiments, the mixture of NTPs comprises modified NTPs. In some embodiments, the modified NTPs comprise N¹-methylpseudouridine, Pseudouridine, N¹-Ethylpseudouridine, N¹-Methoxymethylpseudouridine, N¹-Propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-Bromouridine, 5-lodouridine, 5,6-dihydrouridine, 6-Azauridine, Thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxycytidine, 5-hydroxycytidine, 5-lodocytidine, 5-Bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-Aminoadenosine, N⁶-isopentenyladenosine, N⁶,N⁶-dimethyladenosine, 7-methyladenine, 2-methylthio-adenine, and 2-methoxy-adenine.

In some embodiments, step (a) further comprises providing a capping enzyme. In some embodiments, isolating and purifying transcribed RNAs comprise column purification.

In some aspects, provided herein, is a composition comprising a recombinant RNA construct comprising: (i) a small interfering RNA (siRNA) capable of binding to Interleukin 8 (IL-8) messenger RNA (mRNA); and (ii) an mRNA encoding Insulin-like Growth Factor 1 (IGF-1). In related aspects, the composition comprises or encodes at least 1, 2, or 3 siRNAs.

In some aspects, provided herein, is a composition comprising a recombinant RNA construct comprising: (i) a small interfering RNA (siRNA) capable of binding to Interleukin 1 beta (IL-1 beta) messenger RNA (mRNA); and (ii) an mRNA encoding Insulin-like Growth Factor 1 (IGF-1). In related aspects, the composition comprises or encodes at least 1, 2, or 3 siRNAs.

In some aspects, provided herein, is a composition comprising a recombinant RNA construct comprising: (i) a small interfering RNA (siRNA) capable of binding to Interleukin 17 (IL-17) messenger RNA (mRNA); and (ii) an mRNA encoding Interleukin 4 (IL-4). In related aspects, the composition comprises or encodes at least 1, 2, or 3 siRNAs.

In some aspects, provided herein, is a composition comprising a recombinant RNA construct comprising: (i) a small interfering RNA (siRNA) capable of binding to Tumor Necrosis Factor alpha (TNF-alpha) messenger RNA (mRNA); and (ii) an mRNA encoding Interleukin 4 (IL-4). In related aspects, the composition comprises or encodes at least 1, 2, or 3 siRNAs.

In some aspects, provided herein, is a composition comprising a recombinant RNA construct comprising: (i) a small interfering RNA (siRNA) capable of binding to Tumor Necrosis Factor alpha (TNF-alpha) messenger RNA (mRNA) and a small interfering RNA (siRNA) capable of binding to Interleukin 17 (IL-17) messenger RNA (mRNA); and (ii) an mRNA encoding Interleukin 4 (IL-4). In related aspects, the composition comprises or encodes at least 2, 3, 4, 5, or 6 siRNAs.

In some aspects, provided herein, is a composition comprising a recombinant polynucleic acid construct comprising a nucleic acid sequence selected from the group consisting of SEQ ID NOs: 1-8.

In some aspects, provided herein, is a composition comprising a recombinant polynucleic acid construct encoding or comprising: (i) at least one siRNA capable of binding to IL-6 mRNA; and (ii) an mRNA encoding Interferon beta (IFN-beta). In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6 mRNA. In related aspects, the composition comprises or encodes 3 siRNAs, each directed to IL-6 mRNA. In related aspects, each of the at least 3 siRNAs is the same, different, or a combination thereof. In related aspects, the recombinant polynucleic acid construct comprises a sequence as set forth in SEQ ID NO: 29 or 30 (Compound B1 or B2).

In some aspects, provided herein, is a composition comprising a recombinant polynucleic acid construct encoding or comprising: (i) at least one siRNA capable of binding to Interleukin 6R (IL-6R) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R mRNA. In related aspects, the composition comprises or encodes 3 siRNAs, each directed to IL-6R mRNA. In related aspects, each of the at least 3 siRNAs is the same, different, or a combination thereof. In related aspects, the recombinant polynucleic acid construct comprises a sequence as set forth in SEQ ID NO: 31 (Compound B3).

In some aspects, provided herein, is a composition comprising a recombinant polynucleic acid construct encoding or comprising (i) at least one siRNA capable of binding to Interleukin 6R alpha (IL-6R-alpha) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R-alpha mRNA. In related aspects, the composition comprises or encodes 3 siRNAs, each directed to IL-6R-alpha mRNA. In related aspects, each of the at least 3 siRNAs is the same, different, or a combination thereof. In related aspects, the recombinant polynucleic acid construct comprises a sequence as set forth in SEQ ID NO: 32 (Compound B4).

In some aspects, provided herein, is a composition comprising a recombinant polynucleic acid construct encoding or comprising (i) at least one siRNA capable of binding to Interleukin 6R beta (IL-6R-beta) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R-beta mRNA. In related aspects, the composition comprises or encodes 3 siRNAs, each directed to IL-6R-beta mRNA. In related aspects, each of the at least 3 siRNAs is the same, different, or a combination thereof. In related aspects, the recombinant polynucleic acid construct comprises a sequence as set forth in SEQ ID NO: 33 (Compound B5).

In some aspects, provided herein, is a composition comprising a recombinant polynucleic acid construct encoding or comprising (i) at least one siRNA capable of binding to ACE2 mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to ACE2 mRNA. In related aspects, the composition comprises or encodes 3 siRNAs, each directed to ACE2 mRNA. In related aspects, each of the at least 3 siRNAs is the same, different, or a combination thereof. In related aspects, the recombinant polynucleic acid construct comprises a sequence as set forth in SEQ ID NO: 34 or 35 (Compound B6 or B7).

In some aspects, provided herein, is a composition comprising a recombinant polynucleic acid construct: encoding or comprising (i) at least one siRNA capable of binding to SARS CoV-2 ORF1ab mRNA, at least one siRNA capable of binding to SARS CoV-2 S mRNA, at least one siRNA capable of binding to SARS CoV-2 N mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 3 siRNAs, one directed to SARS CoV-2 ORF1ab mRNA, one directed to SARS CoV-2 S mRNA, and one directed to SARS CoV-2 N mRNA. In related aspects, each of the at least 3 siRNAs is the same, different, or a combination thereof. In certain aspects, such a composition, e.g., a composition comprising Compound B8 (SEQ ID NO: 36) is contemplated for use in methods described herein, e.g., for modulating or regulating gene expression in relation to infection with SARS CoV, SARS CoV-2, or both. In related aspects, the recombinant polynucleic acid construct comprises a sequence as set forth in SEQ ID NO: 36.

In some aspects, provided herein, is a composition comprising a recombinant polynucleic acid construct: encoding or comprising (i) at least one siRNA capable of binding to SARS CoV-2 S mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to SARS CoV-2 S mRNA. In related aspects, the composition comprises or encodes 3 siRNAs, each directed to SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 37 or 39 (Compound B9 or B11).

In some aspects, provided herein, is a composition comprising a recombinant polynucleic acid construct encoding or comprising (i) at least one siRNA capable of binding to SARS CoV-2 N mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to SARS CoV-2 N mRNA. In related aspects, the composition comprises or encodes 3 siRNAs, each directed to SARS CoV-2 N mRNA. In related aspects, each of the at least 3 siRNAs is the same, different, or a combination thereof. In related aspects, the recombinant polynucleic acid construct comprises a sequence as set forth in SEQ ID NO: 38 (Compound B10).

In some aspects, provided herein, is a composition comprising a recombinant polynucleic acid construct: encoding or comprising (i) at least one siRNA capable of binding to SARS CoV-2 ORF1ab mRNA; and (ii) an mRNA encoding IFN-beta. In related aspects, the recombinant polynucleic acid construct in (ii) encodes or further encodes the ACE2 soluble receptor. In related aspects, the composition comprises or encodes 1 siRNA directed to SARS CoV-2 ORF1ab mRNA. In related aspects, the composition comprises or encodes 3 siRNAs, each directed to SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS-CoV, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS-CoV. In related aspects, the recombinant polynucleic acid construct comprises a sequence as set forth in any one of SEQ ID NOs: 40, 41 and 42 (Compounds B12, B13, and B14).

In some aspects, provided herein, is a composition comprising a recombinant polynucleic acid construct: encoding or comprising (i) at least one siRNA capable of binding to IL-6 mRNA, at least one siRNA capable of binding to ACE2 mRNA, and at least one siRNA capable of binding to SARS CoV-2 S mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 3 siRNAs, one directed IL-6 mRNA, one directed to ACE2 mRNA, and one directed to SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the recombinant polynucleic acid construct comprises a sequence as set forth in any one of SEQ ID NOs: 43, 44, and 45 (Compounds B15, B16, and B17).

In some aspects, provided herein, is a composition comprising a recombinant polynucleic acid construct encoding or comprising (i) at least one small interfering RNA capable of binding to SARS CoV-2 ORF1ab mRNA, at least one siRNA capable of binding to SARS CoV-2 S mRNA, and at least one siRNA capable of binding to SARS CoV-2 N mRNA; and (ii) an mRNA encoding the ACE2 soluble receptor. In related aspects, the composition comprises or encodes at least 1, 2, or 3 siRNAs. In related aspects, the composition comprises or encodes 3 siRNAs, one directed to ORF1ab mRNA, one directed to SARS CoV-2 S mRNA, and one directed to SARS CoV-2 N mRNA. In related aspects, the recombinant polynucleic acid construct comprises a sequence as set forth in SEQ ID NO: 46 (Compound B18). In related aspects, the recombinant polynucleic acid construct comprises a sequence as set forth in SEQ ID NO: 190 (Compound B18).

In some aspects, provided herein, is a composition comprising a recombinant polynucleic acid construct: encoding or comprising (i) at least one siRNA capable of binding to SARS CoV-2 S mRNA; and (ii) an mRNA encoding the ACE2 soluble receptor. In related aspects, the composition comprises or encodes at least 1, 2, or 3 siRNAs. In related aspects, the composition comprises or encodes 1 siRNA directed to SARS CoV-2 S mRNA. In related aspects, the composition comprises or encodes 3 siRNAs, each directed to SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 47 (Compound B19).

In some aspects, provided herein, is a composition comprising a recombinant RNA construct comprising: (i) at least one siRNA capable of binding to an IL-6 mRNA; and (ii) an mRNA encoding interferon-beta (IFN-beta). In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to an IL-6 mRNA. In related aspects, the recombinant RNA construct comprises 3 siRNAs, each directed to an IL-6 mRNA. In related aspects, each of the at least 3 siRNAs is the same, different, or a combination thereof. In related aspects, the recombinant RNA construct comprises a sequence encoded by a sequence as set forth in SEQ ID NO: 29 or 30.

In some aspects, provided herein, is a composition comprising a recombinant RNA construct comprising: (i) at least one siRNA capable of binding to an Interleukin 6R (IL-6R) mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to an IL-6R mRNA. In related aspects, the recombinant RNA construct comprises 3 siRNAs, each directed to an IL-6R mRNA. In related aspects, each of the at least 3 siRNAs is the same, different, or a combination thereof. In related aspects, the recombinant RNA construct comprises a sequence encoded by a sequence as set forth in SEQ ID NO: 31.

In some aspects, provided herein, is a composition comprising a recombinant RNA construct comprising: (i) at least one siRNA capable of binding to an Interleukin 6R alpha (IL-6R-alpha) mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to an IL-6R-alpha mRNA. In related aspects, the recombinant RNA construct comprises 3 siRNAs, each directed to an IL-6R-alpha mRNA. In related aspects, each of the at least 3 siRNAs is the same, different, or a combination thereof. In related aspects, the recombinant RNA construct comprises a sequence encoded by the sequence as set forth in SEQ ID NO: 32.

In some aspects, provided herein, is a composition comprising a recombinant RNA construct comprising: (i) at least one siRNA capable of binding to an Interleukin 6R beta (IL-6R-beta) mRNA; and (ii) an mRNA encoding IFN-beta. In related aspects, the mRNA of ii) encodes an ACE2 soluble receptor. In related aspects, the recombinant RNA construct comprises at least 1, 2, or 3 siRNAs. In related aspects, the recombinant RNA construct comprises 1 siRNA directed to an IL-6R-beta mRNA. In related aspects, the recombinant RNA construct comprises 3 siRNAs, each directed to an IL-6R-beta mRNA. In related aspects, each of the at least 3 siRNAs is the same, different, or a combination thereof. In related aspects, the recombinant RNA construct comprises a sequence encoded by the sequence as set forth in SEQ ID NO: 33.

In some aspects, provided herein, is a composition comprising a recombinant RNA construct comprising: (i) at least one siRNA capable of binding to an ACE2 mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to an ACE2 mRNA. In related aspects, the recombinant RNA construct comprises 3 siRNAs, each directed to an ACE2 mRNA. In related aspects, each of the at least 3 siRNAs is the same, different, or a combination thereof. In related aspects, the recombinant RNA construct comprises a sequence encoded by a sequence as set forth in SEQ ID NO: 34 or 35.

In some aspects, provided herein, is a composition comprising a recombinant RNA construct comprising: (i) at least one siRNA capable of binding to a SARS CoV-2 ORF1ab mRNA, at least one siRNA capable of binding to a SARS CoV-2 S mRNA, at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 3 siRNAs, one directed to a SARS CoV-2 ORF1ab mRNA, one directed to a SARS CoV-2 S mRNA, and one directed to a SARS CoV-2 N mRNA. In related aspects, each of the at least 3 siRNAs is the same, different, or a combination thereof. In certain aspects, such a composition, e.g., a composition comprising Compound B8 (SEQ ID NO: 36), is contemplated for use in methods described herein, e.g., for modulating or regulating gene expression in relation to infection with SARS CoV, SARS CoV-2, or both. In related aspects, the recombinant RNA construct comprises a sequence encoded by the sequence as set forth in SEQ ID NO: 36.

In some aspects, provided herein, is a composition comprising a recombinant RNA construct comprising: (i) at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to a SARS CoV-2 S mRNA. In related aspects, the recombinant RNA construct comprises 3 siRNAs, each directed to a SARS CoV-2 S mRNA. In related aspects, each of the at least 3 siRNAs is the same, different, or a combination thereof. In related aspects, the recombinant RNA construct comprises a sequence encoded by a sequence as set forth in SEQ ID NO: 37 or 39.

In some aspects, provided herein, is a recombinant RNA construct comprising a recombinant RNA construct comprising: (i) at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 1 siRNA directed to a SARS CoV-2 N mRNA. In related aspects, the recombinant RNA construct comprises 3 siRNAs, each directed to a SARS CoV-2 N mRNA. In related aspects, each of the at least 3 siRNAs is the same, different, or a combination thereof. In related aspects, the recombinant RNA construct comprises a sequence encoded by the sequence as set forth in SEQ ID NO: 38.

In some aspects, provided herein, is a composition comprising a recombinant RNA construct comprising: (i) at least one siRNA capable of binding to a SARS CoV-2 ORF1ab mRNA; and (ii) an mRNA encoding IFN-beta. In related aspects, the mRNA of ii) encodes or further encodes an ACE2 soluble receptor. In related aspects, the recombinant RNA construct comprises 1 siRNA directed to a SARS CoV-2 ORF1ab mRNA. In related aspects, the recombinant RNA construct comprises 3 siRNAs, each directed to a SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS. In related aspects, the recombinant RNA construct comprises a sequence encoded by a sequence as set forth in any one of SEQ ID NOs: 40, 41 and 42.

In some aspects, provided herein, is a composition comprising a recombinant RNA construct comprising: (i) at least one siRNA capable of binding to an IL-6 mRNA, at least one siRNA capable of binding to an ACE2 mRNA, and at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the recombinant RNA construct comprises at least 1, 2, or 3 siRNAs. In related aspects, the recombinant RNA construct comprises 3 siRNAs, one directed to an IL-6 mRNA, one directed to an ACE2 mRNA, and one directed to a SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the recombinant RNA construct comprises a sequence encoded by a sequence as set forth in any one of SEQ ID NOs: 43, 44, and 45.

In some aspects, provided herein, is a composition comprising a recombinant RNA construct comprising: (i) at least one small interfering RNA capable of binding to a SARS CoV-2 ORF1ab mRNA, at least one siRNA capable of binding to a SARS CoV-2 S mRNA, and at least one siRNA capable of binding to a SARS CoV-2 N mRNA; and (ii) an mRNA encoding an ACE2 soluble receptor. In related aspects, the recombinant RNA construct comprises at least 1, 2, or 3 siRNAs. In related aspects, the recombinant RNA construct comprises 3 siRNAs, one directed to an ORF1ab mRNA, one directed to a SARS CoV-2 S mRNA, and one directed to a SARS CoV-2 N mRNA. In related aspects, the recombinant RNA construct comprises a sequence encoded by the sequence as set forth in SEQ ID NO: 46.

In some aspects, provided herein, is a composition comprising a recombinant RNA construct comprising: (i) at least one siRNA capable of binding to a SARS CoV-2 S mRNA; and (ii) an mRNA encoding an ACE2 soluble receptor. In related aspects, the recombinant RNA construct comprises at least 1, 2, or 3 siRNAs. In related aspects, the recombinant RNA construct comprises 1 siRNA directed to a SARS CoV-2 S mRNA. In related aspects, the recombinant RNA construct comprises 3 siRNAs, each directed to a SARS CoV-2 S mRNA. In related aspects, each of the at least 3 siRNAs is the same, different, or a combination thereof.

In related aspects, the recombinant RNA construct comprises a sequence encoded by the sequence as set forth in SEQ ID NO: 47.

In some aspects, the present invention provides a composition comprising a recombinant RNA construct comprising a nucleic acid sequence encoded by a sequence selected from the group consisting of SEQ ID NOs: 29-47.

In some embodiments, a polynucleic acid construct of the present invention comprises: (i) an siRNA that targets an RNA selected from: an IL-8 mRNA, an IL-1 beta mRNA, an IL-17 mRNA, a TNF-alpha mRNA, a SARS CoV-2 ORF1ab RNA (polyprotein PP1ab, e.g., in a noncoding region or where it encodes a protein that is selected from: a SARS CoV-2 nonstructure protein (NSP), Nsp1, Nsp3 (Nsp3b, Nsp3c, PLpro, and Nsp3e), Nsp7_Nsp8 complex, Nsp9-Nsp10, and Nsp14-Nsp16, 3CLpro, E-channel (E protein), ORF7a, C-terminal RNA binding domain (CRBD), N-terminal RNA binding domain (NRBD), helicase, and RdRp), a SARS CoV-2 Spike protein (S) mRNA, a SARS CoV-2 Nucleocapsid protein (N) mRNA, a tumor necrosis factor alpha (TNF-alpha) mRNA, an interleukin mRNA (including but not limited to interleukin 1 (e.g., IL-1alpha, IL-1beta), interleukin 6 (IL-6), interleukin 6R (IL-6R), interleukin 6R alpha (IL-6R-alpha), interleukin 6R beta (IL-6R-beta), interleukin 18 (IL-18), interleukin 36-alpha (IL-36-alpha), interleukin 36-beta (IL-36-beta), interleukin 36-gamma (IL-36-gamma), interleukin 33 (IL-33)), an Angiotensin Converting Enzyme-2 (ACE2) mRNA, a transmembrane protease, serine 2 (TMPRSS2) mRNA, and a coding NSP12 and 13 RNA; and (ii) at least one gene of interest that encodes, or at least one mRNA that encodes, a protein to be overexpressed, wherein the protein is selected from: IGF-1, IL-4, IGF-1 (including derivatives thereof as described elsewhere herein), carboxypeptidases (e.g., ACE, ACE2, CNDP1, CPA1, CPA2, CPA4, CPA5, CPA6, CPB1, CPB2, CPE, CPN1, CPQ, CPXM1, CPZ, SCPEP1); cytokines (e.g., BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2); extracellular ligands and transporters (e.g., APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, VWC2L); extracellular matrix proteins (e.g., ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLN5, FLRT1, FLRT2, FLRT3, FREM1, GLDN, IBSP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR, TNXB); glucosidases (AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACA5, SPACA5B); glycosyltransferases (e.g., ARTS, B4GALT1, EXTL2, GALNT1, GALNT2, GLT1D1, MGAT4A, ST3GAL1, ST3GAL2, ST3GAL3, ST3GAL4, ST6GAL1, XYLT1); growth factors (e.g., AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF-1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, WISP3); growth factor binding proteins (e.g., CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1); heparin binding proteins (e.g., ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, VTN); hormones (e.g., ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGB5, CGB8, COPA, CORT, CRH, CSH1, CSH2, CSHL1, ENHO, EPO, ERFE, FBN1, FNDC5, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP); hydrolases (e.g., AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPP5, ENTPD5, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4); immunoglobulins (e.g., IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, IGLC3); isomerases (e.g., NAXE, PPIA, PTGDS); kinases (e.g., ADCK1, ADPGK, FAM20C, ICOS, PKDCC); lyases (e.g., PM20D1, PAM, CA6); metalloenzyme inhibitors (e.g., FETUB, SPOCK3, TIMP2, TIMP3, TIMP4, WFIKKN1, WFIKKN2); metalloproteases (e.g., ADAM12, ADAM28, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMP1, MMP8, MMP9, PAPPA, PAPPA2, TLL1, TLL2); milk proteins (e.g., CSN1S1, CSN2, CSN3, LALBA); neuroactive proteins (e.g., CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3); proteases (e.g., ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSK5, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, TPSD1); protease inhibitors (e.g., A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINB5, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, WFDC8); protein phosphatases (e.g., ACP7, ACPP, PTEN, PTPRZ1); esterases (e.g., BCHE, CEL, CES4A, CES5A, NOTUM, SIAE); transferases (e.g., METTL24, FKRP, CHSY1, CHST9, B3GAT1); vasoactive proteins (e.g., AGGF1, AGT, ANGPT1, ANGPT2, ANGPTL4, ANGPTL6, EDN1, EDN2, EDN3, NTS), a Type I interferon (e.g., an IFN-α, including, but not limited to an interferon alpha-n3, an interferon alpha-2a, and an interferon alpha-2b, an IFN-β, an IFN-δ, an IFN-ε, an IFN-κ, an IFN-ν, an IFN-τ, and an IFN-ω), a Type II interferon (e.g., IFN-γ), a Type III interferon (e.g., IFN-λ) an interleukin, e.g., IL-37, IL-38, and a soluble ACE2 receptor.

In some aspects, provided herein, is a composition comprising a recombinant polynucleic acid construct comprising: (i) at least one nucleic acid sequence encoding or comprising a small interfering RNA (siRNA) capable of binding to a target RNA; and (ii) at least one nucleic acid sequence encoding a gene of interest; wherein the target RNA is different from an mRNA encoded by the gene of interest. In some aspects, the recombinant polynucleic acid construct comprises two or more nucleic acid sequences encoding or comprising an siRNA capable of binding to a target RNA, wherein each of the two or more nucleic acid sequences encode or comprise an siRNA capable of binding to a same target RNA or a different target RNA. In some embodiments, the recombinant polynucleic acid construct comprises two or more nucleic acid sequences that each encode or comprise an siRNA capable of binding to a target RNA, wherein the respective target RNAs are the same, different, or a combination thereof. In some embodiments, the target RNA is an mRNA. In some embodiments, the target RNA is a noncoding RNA. In some aspects, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some aspects, the at least one nucleic acid sequence encoding or comprising a small interfering RNA (siRNA) capable of binding to a target RNA (i) and the at least one nucleic acid sequence encoding a gene of interest (ii) are present in a sequential manner. In some aspects, the nucleic acid sequence encoding or comprising a small interfering RNA (siRNA) capable of binding to a target RNA (i) is upstream of the at least one nucleic acid sequence encoding a gene of interest (ii). In some aspects, the nucleic acid sequence encoding or comprising a small interfering RNA (siRNA) capable of binding to a target RNA (i) is downstream of the at least one nucleic acid sequence encoding a gene of interest (ii). In some aspects, the nucleic acid sequence encoding or comprising a small interfering RNA (siRNA) capable of binding to a target RNA (i) is upstream or downstream of the at least one nucleic acid sequence encoding a gene of interest (ii). In some aspects, the siRNA capable of binding to a target RNA binds to an exon of a target mRNA. In some aspects, the siRNA capable of binding to a target RNA specifically binds to one target RNA. In some aspects, the siRNA capable of binding to a target RNA is not encoded by or comprised of an intron sequence of the gene of interest. In some aspects, the gene of interest is expressed without RNA splicing. In some aspects, the target RNA is an mRNA encoding a protein selected from the group consisting of Tumor Necrosis Factor alpha (TNF-alpha), interleukin, Angiotensin Converting Enzyme-2 (ACE2), SARS CoV-2 ORF1ab, SARS CoV-2 S, and SARS CoV-2 N. In some aspects, the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Interleukin 6 (IL-6), Interleukin 6R (IL-6R), Interleukin 6R-alpha (IL-6R-alpha), Interleukin 6R-beta (IL-6R-beta), Angiotensin Converting Enzyme-2 (ACE2), SARS CoV-2 ORF1ab, SARS CoV-2 S, and SARS CoV-2 N. In some aspects, the target RNA is an mRNA encoding a protein selected from the group consisting of: Interleukin 8 (IL-8), Interleukin 1 beta (IL-1 beta), Interleukin 17 (IL-17), and Tumor Necrosis Factor alpha (TNF-alpha). In some aspects, the recombinant polynucleic acid construct comprises two or more nucleic acid sequences encoding a gene of interest, wherein each of the two or more nucleic acid sequences encodes a same gene of interest or a different gene of interest. In some embodiments, the recombinant polynucleic acid construct comprises two or more nucleic acid sequences that each encode or a gene of interest, wherein the respective genes of interest are the same, different, or a combination thereof. In some aspects, the gene of interest comprises a nucleic acid sequence encoding a protein selected from the group consisting of a secretory protein, an intracellular protein, an intraorganelle protein, and a membrane protein. In some aspects, the gene of interest is selected from the group consisting of Insulin-like Growth Factor 1 (IGF-1), and Interleukin 4 (IL-4). In some aspects, the recombinant polynucleic acid construct further comprises a nucleic acid sequence encoding a target motif operably linked to the at least one nucleic acid sequence encoding the gene of interest, wherein the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some aspects, the target motif is selected from the group consisting of: (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid. In some aspects, the recombinant polynucleic acid construct further comprises a nucleic acid sequence encoding or comprising a poly(A) tail, a nucleic acid sequence encoding or comprising a 5′ cap, a nucleic acid sequence encoding or comprising a promoter, or a nucleic acid sequence encoding or comprising a Kozak sequence. In some aspects, the recombinant polynucleic acid construct further comprises a nucleic acid sequence encoding or comprising a linker. In some aspects, the nucleic acid sequence encoding or comprising the linker connects (a) the at least one nucleic acid sequence encoding or comprising an siRNA capable of binding to a target mRNA and the at least one nucleic acid sequence encoding a gene of interest, (b) each of the two or more nucleic acid sequences encoding or comprising an siRNA capable of binding to a target mRNA, and/or (c) each of the two or more nucleic acid sequences encoding a gene of interest. In some aspects, the linker comprises a tRNA linker, a 2A peptide linker or a flexible linker. In some aspects, the linker is at least 6 nucleic acid residues in length. In some aspects, the nucleic acid sequence encoding or comprising the linker is up to 50 nucleic acid residues in length. In some aspects, the nucleic acid sequence encoding or comprising the linker is up to 80 nucleic acid residues in length. In some aspects, the nucleic acid sequence encoding or comprising the linker is about 6 to about 50 nucleic acid residues in length. In some aspects, the nucleic acid sequence encoding or comprising the linker is about 6 to about 80 nucleic acid residues in length. In some aspects, the nucleic acid sequence encoding or comprising the linker is about 6 to about 15 nucleic acid residues in length. In some aspects, the recombinant polynucleic acid construct comprises a nucleic acid sequence selected from the group consisting of SEQ ID NOs: 1-8. In some aspects, the composition comprises a recombinant RNA construct comprising: (i) a small interfering RNA (siRNA) capable of binding to a target RNA; and (ii) an mRNA encoding a gene of interest; wherein the target RNA is different from the mRNA encoding the gene of interest. In some aspects, the composition is for use in simultaneously modulating the expression of two or more genes in a cell. In some aspects, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some aspects, the composition is for use in simultaneously modulating the expression of two or more genes in a cell. In some aspects, the at least one nucleic acid sequence encoding or comprising a small interfering RNA (siRNA) capable of binding to a target RNA (i) and the at least one nucleic acid sequence encoding a gene of interest (ii) are present in a sequential manner. In some aspects, the nucleic acid sequence encoding or comprising a small interfering RNA (siRNA) capable of binding to a target RNA (i) is upstream of the at least one nucleic acid sequence encoding a gene of interest (ii). In some aspects, the nucleic acid sequence encoding or comprising a small interfering RNA (siRNA) capable of binding to a target RNA (i) is downstream of the at least one nucleic acid sequence encoding a gene of interest (ii). In some aspects, the nucleic acid sequence encoding or comprising a small interfering RNA (siRNA) capable of binding to a target RNA (i) is upstream or downstream of the at least one nucleic acid sequence encoding a gene of interest (ii). In some aspects, the siRNA capable of binding to a target RNA binds to an exon of a target mRNA. In some aspects, the siRNA capable of binding to a target RNA specifically binds to one target RNA. In some aspects, the siRNA capable of binding to a target RNA is not encoded by or comprised of an intron sequence of the gene of interest. In some aspects, the gene of interest is expressed without RNA splicing.

In some aspects, provided herein, is a composition comprising a recombinant polynucleic acid construct for treatment or prevention of a viral disease or condition in a subject, the construct comprising: (i) at least one nucleic acid sequence encoding or comprising a small interfering RNA (siRNA) capable of binding to a target RNA; and (ii) at least one nucleic acid sequence encoding or comprising an mRNA of a gene of interest; wherein the target RNA is different from an mRNA encoded by the gene of interest. In some aspects, the siRNA does not affect the expression of and/or is not capable of binding to the mRNA of the gene of interest. In some aspects, the recombinant polynucleic acid construct comprises two or more nucleic acid sequences encoding or comprising an siRNA capable of binding to a target RNA, wherein each of the two or more nucleic acid sequences encode or comprise an siRNA capable of binding to a same target RNA or a different target RNA. In some aspects, the recombinant polynucleic acid construct comprises three or more nucleic acid sequences encoding or comprising an siRNA capable of binding to a target RNA, wherein at least two nucleic acid sequences encode or comprise an siRNA capable of binding to the same target RNA and at least one nucleic acid sequence encodes or comprises an siRNA capable of binding to a different target RNA. In some embodiments, the target RNA is an mRNA. In some embodiments, the target RNA is a noncoding RNA. In some embodiments, each target RNA is the same, or different. In some embodiments, the target RNA is an mRNA encoding a protein selected from the group consisting of: interleukin, Angiotensin Converting Enzyme-2 (ACE2); SARS CoV-2 ORF1ab; SARS CoV-2 S, and SARS CoV-2 N. In some embodiments, the interleukin is selected from the group consisting of: IL-1alpha, IL-1beta, IL-6, IL-6R, IL-6R-alpha, interleukin IL-6R-beta, IL-18, IL-36-alpha, IL-36-beta; IL-36-gamma, and IL-33. In some embodiments, the target mRNA is an mRNA encoding a protein selected from the group consisting of: IL-6, IL-6R, IL-6R-alpha, IL-6R-beta, Angiotensin Converting Enzyme-2 (ACE2); SARS CoV-2 ORF1ab; SARS CoV-2 S, and SARS CoV-2 N. In some embodiments, the composition comprises in (ii) two or more nucleic acid sequences, each encoding a gene of interest. In some embodiments, each mRNA is the same or different. In some embodiments, at least two mRNAs are the same and at least one mRNA is different from the at least two same mRNAs. In some embodiments, the gene of interest of (ii) is selected from the group of genes encoding: IFN alpha-n3, IFN alpha-2a, IFN alpha-2b, IFN beta-1a, IFN beta-1b, ACE2 soluble receptor, IL-37, and IL-38. In some embodiments, the gene of interest of (ii) is selected from the group of genes encoding: IFN beta and ACE2 soluble receptor. In some embodiments, the recombinant polynucleic acid construct further comprises a nucleic acid sequence encoding a target motif operably linked to the at least one nucleic acid sequence encoding the mRNA of the gene of interest, wherein the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some embodiments, the target motif is selected from the group consisting of: (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid. In some embodiments, the recombinant polynucleic acid construct further comprises a nucleic acid sequence encoding or comprising a poly(A) tail, a nucleic acid sequence encoding or comprising a 5′ cap, a nucleic acid sequence encoding or comprising a promoter, or a nucleic acid sequence encoding or comprising a Kozak sequence. In some embodiments, the recombinant polynucleic acid construct further comprises a nucleic acid sequence encoding or comprising a linker. In some embodiments, the nucleic acid sequence encoding or comprising the linker connects (a) the at least one nucleic acid sequence encoding or comprising the siRNA capable of binding to the target mRNA and the at least one nucleic acid sequence encoding the gene of interest, (b) each of the two or more nucleic acid sequences encoding or comprising the siRNA capable of binding to the target mRNA, and/or (c) each of the two or more nucleic acid sequences encoding the gene of interest. In some embodiments, the linker comprises a tRNA linker, a 2A peptide linker, or a flexible linker. In some aspects, the nucleic acid sequence encoding or comprising the linker is at least 6 nucleic acid residues in length. In some aspects, the nucleic acid sequence encoding or comprising the linker is up to 50 nucleic acid residues in length. In some aspects, the nucleic acid sequence encoding or comprising the linker is up to 80 nucleic acid residues in length. In some aspects, the nucleic acid sequence encoding or comprising the linker is about 6 to about 50 nucleic acid residues in length. In some aspects, the nucleic acid sequence encoding or comprising the linker is about 6 to about 80 nucleic acid residues in length. In some aspects, the nucleic acid sequence encoding or comprising the linker is about 6 to about 15 nucleic acid residues in length. In some embodiments, the recombinant polynucleic acid construct is a vector suitable for gene therapy. In some embodiments, the recombinant polynucleic acid construct comprises a nucleic acid sequence selected from the group consisting of SEQ ID NOs: 29-47. In some aspects, the composition comprises a recombinant RNA construct comprising: (i) a small interfering RNA (siRNA) capable of binding to a target RNA; and (ii) an mRNA encoding a gene of interest; wherein the target RNA is different from the mRNA encoding the gene of interest. In some embodiments, the composition is for use in simultaneously modulating the expression of two or more genes in a cell. In some embodiments, the composition is present in an amount sufficient to treat or prevent a viral disease or condition in the subject. In some aspects, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some aspects, the at least one nucleic acid sequence encoding or comprising a small interfering RNA (siRNA) capable of binding to a target RNA (i) and the at least one nucleic acid sequence encoding a gene of interest (ii) are present in a sequential manner. In some aspects, the nucleic acid sequence encoding or comprising a small interfering RNA (siRNA) capable of binding to a target RNA (i) is upstream of the at least one nucleic acid sequence encoding a gene of interest (ii). In some aspects, the nucleic acid sequence encoding or comprising a small interfering RNA (siRNA) capable of binding to a target RNA (i) is downstream of the at least one nucleic acid sequence encoding a gene of interest (ii). In some aspects, the nucleic acid sequence encoding or comprising a small interfering RNA (siRNA) capable of binding to a target RNA (i) is upstream or downstream of the at least one nucleic acid sequence encoding a gene of interest (ii). In some aspects, the siRNA capable of binding to a target RNA binds to an exon of a target mRNA. In some aspects, the siRNA capable of binding to a target RNA specifically binds to one target RNA. In some aspects, the siRNA capable of binding to a target RNA is not encoded by or comprised of an intron sequence of the gene of interest. In some aspects, the gene of interest is expressed without RNA splicing.

In some aspects, provided herein, is a composition comprising a recombinant polynucleic acid construct, the construct comprising: (i) at least one nucleic acid sequence encoding or comprising a small interfering RNA (siRNA) capable of binding to a target RNA; and (ii) at least one nucleic acid sequence encoding or comprising an mRNA of a gene of interest; wherein the target RNA of (i) is different from the mRNA of (ii). In some embodiments, the siRNA does not affect the expression of and/or is not capable of binding to the mRNA of the gene of interest. In some aspects, the recombinant polynucleic acid construct comprises two or more nucleic acid sequences encoding or comprising an siRNA capable of binding to a target RNA, wherein each of the two or more nucleic acid sequences encode or comprise an siRNA capable of binding to a same target RNA or a different target RNA. In some aspects, the recombinant polynucleic acid construct comprises three or more nucleic acid sequences encoding or comprising an siRNA capable of binding to a target RNA, wherein at least two nucleic acid sequences encode or comprise an siRNA capable of binding to the same target RNA and at least one nucleic acid sequence encodes or comprises an siRNA capable of binding to a different target RNA. In some embodiments, the target RNA is an mRNA. In some embodiments, the target RNA is a noncoding RNA. In some embodiments, each target RNA is the same, or different. In some embodiments, the target is an mRNA encoding a protein selected from the group consisting of: IL-8 mRNA, an IL-1 beta mRNA, an IL-17 mRNA, a TNF-alpha mRNA, a SARS CoV-2 ORF1ab RNA (polyprotein PP1ab, e.g., in a noncoding region or where it encodes a protein that is selected from: a SARS CoV-2 nonstructure protein (NSP), Nsp1, Nsp3 (Nsp3b, Nsp3c, PLpro, and Nsp3e), Nsp7 Nsp8 complex, Nsp9-Nsp10, and Nsp14-Nsp16, 3CLpro, E-channel (E protein), ORF7a, C-terminal RNA binding domain (CRBD), N-terminal RNA binding domain (NRBD), helicase, and RdRp), a SARS CoV-2 Spike protein (S) mRNA, a SARS CoV-2 Nucleocapsid protein (N) mRNA, a tumor necrosis factor alpha (TNF-alpha) mRNA, an interleukin mRNA (including but not limited to interleukin 1 (e.g., IL-1alpha, IL-1beta), interleukin 6 (IL-6), interleukin 6R (IL-6R), interleukin 6R alpha (IL-6R-alpha), interleukin 6R beta (IL-6R-beta), interleukin 18 (IL-18), interleukin 36-alpha (IL-36-alpha), interleukin 36-beta (IL-36-beta), interleukin 36-gamma (IL-36-gamma), interleukin 33 (IL-33)), an Angiotensin Converting Enzyme-2 (ACE2) mRNA, a transmembrane protease, serine 2 (TMPRSS2) mRNA, and a coding NSP12 and 13 RNA. In some embodiments, the composition comprises in (ii) two or more nucleic acid sequences, each encoding an mRNA of a gene of interest. In some embodiments, each mRNA is the same or different. In some embodiments, at least two mRNAs are the same and at least one mRNA is different from the at least two same mRNAs. In some embodiments, the gene of interest of (ii) is selected from the group of genes encoding a protein selected from: IGF-1, IL-4, IGF-1 (including derivatives thereof as described elsewhere herein), carboxypeptidases (e.g., ACE, ACE2, CNDP1, CPA1, CPA2, CPA4, CPA5, CPA6, CPB1, CPB2, CPE, CPN1, CPQ, CPXM1, CPZ, SCPEP1); cytokines (e.g., BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2); extracellular ligands and transporters (e.g., APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, VWC2L); extracellular matrix proteins (e.g., ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLN5, FLRT1, FLRT2, FLRT3, FREM1, GLDN, IBSP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR, TNXB); glucosidases (AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACA5, SPACA5B); glycosyltransferases (e.g., ARTS, B4GALT1, EXTL2, GALNT1, GALNT2, GLT1D1, MGAT4A, ST3GAL1, ST3GAL2, ST3GAL3, ST3GAL4, ST6GAL1, XYLT1); growth factors (e.g., AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF-1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, WISP3); growth factor binding proteins (e.g., CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1); heparin binding proteins (e.g., ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, VTN); hormones (e.g., ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGB5, CGB8, COPA, CORT, CRH, CSH1, CSH2, CSHL1, ENHO, EPO, ERFE, FBN1, FNDC5, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP); hydrolases (e.g., AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPP5, ENTPD5, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4); immunoglobulins (e.g., IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, IGLC3); isomerases (e.g., NAXE, PPIA, PTGDS); kinases (e.g., ADCK1, ADPGK, FAM20C, ICOS, PKDCC); lyases (e.g., PM20D1, PAM, CA6); metalloenzyme inhibitors (e.g., FETUB, SPOCK3, TIMP2, TIMP3, TIMP4, WFIKKN1, WFIKKN2); metalloproteases (e.g., ADAM12, ADAM28, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMP1, MMP8, MMP9, PAPPA, PAPPA2, TLL1, TLL2); milk proteins (e.g., CSN1S1, CSN2, CSN3, LALBA); neuroactive proteins (e.g., CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3); proteases (e.g., ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSK5, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, TPSD1); protease inhibitors (e.g., A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINB5, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, WFDC8); protein phosphatases (e.g., ACP7, ACPP, PTEN, PTPRZ1); esterases (e.g., BCHE, CEL, CES4A, CES5A, NOTUM, SIAE); transferases (e.g., METTL24, FKRP, CHSY1, CHST9, B3GAT1); vasoactive proteins (e.g., AGGF1, AGT, ANGPT1, ANGPT2, ANGPTL4, ANGPTL6, EDN1, EDN2, EDN3, NTS), a Type I interferon (e.g., an IFN-α, including, but not limited to an interferon alpha-n3, an interferon alpha-2a, and an interferon alpha-2b, an IFN-β, an IFN-δ, an IFN-ε, an IFN-κ, an IFN-ν, an IFN-τ, and an IFN-ω), a Type II interferon (e.g., IFN-γ), a Type III interferon (e.g., IFN-λ), an interleukin, e.g., IL-37, IL-38, and a soluble ACE2 receptor. In some embodiments, the recombinant polynucleic acid construct further comprises a nucleic acid sequence encoding a target motif operably linked to the at least one nucleic acid sequence encoding the mRNA of the gene of interest, wherein the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some embodiments, the target motif is selected from the group consisting of: (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid. In some embodiments, the recombinant polynucleic acid construct further comprises a nucleic acid sequence encoding or comprising a poly(A) tail, a nucleic acid sequence encoding or comprising a 5′ cap, a nucleic acid sequence encoding or comprising a promoter, or a nucleic acid sequence encoding or comprising a Kozak sequence. In some embodiments, the recombinant polynucleic acid construct further comprises a nucleic acid sequence encoding or comprising a linker. In some embodiments, the nucleic acid sequence encoding or comprising the linker connects (a) the at least one nucleic acid sequence encoding or comprising the siRNA capable of binding to the target mRNA and the at least one nucleic acid sequence encoding the gene of interest, (b) each of the two or more nucleic acid sequences encoding or comprising the siRNA capable of binding to the target mRNA, and/or (c) each of the two or more nucleic acid sequences encoding the gene of interest. In some embodiments, the linker comprises a tRNA linker, a 2A peptide linker, or a flexible linker. In some embodiments, the nucleic acid sequence encoding or comprising the linker is at least 6 nucleic acid residues in length. In some embodiments, the nucleic acid sequence encoding or comprising the linker is up to 50 nucleic acid residues in length. In some embodiments, the nucleic acid sequence encoding or comprising the linker is up to 80 nucleic acid residues in length. In some embodiments, the nucleic acid sequence encoding or comprising the linker is about 6 to about 50 nucleic acid residues in length. In some embodiments, the nucleic acid sequence encoding or comprising the linker is about 6 to about 80 nucleic acid residues in length. In some embodiments, the nucleic acid sequence encoding or comprising the linker is about 6 to about 15 nucleic acid residues in length. In some embodiments, the recombinant polynucleic acid construct is a vector suitable for gene therapy. In some embodiments, the composition is useful for simultaneously modulating the expression of two or more genes in a cell.

In some embodiments, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some embodiments, the at least one nucleic acid sequence encoding or comprising a small interfering RNA (siRNA) capable of binding to a target RNA (i) and the at least one nucleic acid sequence encoding a gene of interest (ii) are present in a sequential manner. In some embodiments, the nucleic acid sequence encoding or comprising a small interfering RNA (siRNA) capable of binding to a target RNA (i) is upstream of the at least one nucleic acid sequence encoding a gene of interest (ii). In some embodiments, the nucleic acid sequence encoding or comprising a small interfering RNA (siRNA) capable of binding to a target RNA (i) is downstream of the at least one nucleic acid sequence encoding a gene of interest (ii). In some embodiments, the nucleic acid sequence encoding or comprising a small interfering RNA (siRNA) capable of binding to a target RNA (i) is upstream or downstream of the at least one nucleic acid sequence encoding a gene of interest (ii). In some embodiments, the siRNA capable of binding to a target RNA binds to an exon of a target mRNA. In some embodiments, the siRNA capable of binding to a target RNA specifically binds to one target RNA. In some embodiments, the siRNA capable of binding to a target RNA is not encoded by or comprised of an intron sequence of the gene of interest. In some embodiments, the gene of interest is expressed without RNA splicing. In some embodiments, the composition is present or administered in an amount sufficient to treat or prevent a viral infection, disease or condition, or a disease or condition selected from the group consisting of intervertebral disc disease (IVDD), osteoarthritis, and psoriasis. In some embodiments, the composition is present or administered in an amount sufficient to treat or prevent a viral infection, disease or condition, or a disease or condition selected from the group consisting of intervertebral disc disease (IVDD), osteoarthritis, and psoriasis, fibrodysplasia ossificans progressiva (FOP), and amyotrophic lateral sclerosis (ALS), In some embodiments, the at least one nucleic acid sequence encoding or comprising a small interfering RNA (siRNA) capable of binding to a target RNA (i) and the at least one nucleic acid sequence encoding a gene of interest (ii) are comprised in a sequential manner. In some embodiments, the at least one nucleic acid sequence encoding or comprising a small interfering RNA (siRNA) capable of binding to a target RNA (i) and the at least one nucleic acid sequence encoding a gene of interest (ii) are present in a sequential manner. In some embodiments, the nucleic acid sequence encoding or comprising a small interfering RNA (siRNA) capable of binding to a target RNA (i) is upstream of the at least one nucleic acid sequence encoding a gene of interest (ii). In some embodiments, the nucleic acid sequence encoding or comprising a small interfering RNA (siRNA) capable of binding to a target RNA (i) is downstream of the at least one nucleic acid sequence encoding a gene of interest (ii). In some embodiments, the nucleic acid sequence encoding or comprising a small interfering RNA (siRNA) capable of binding to a target RNA (i) is upstream or downstream of the at least one nucleic acid sequence encoding a gene of interest (ii). In some embodiments, the siRNA capable of binding to a target RNA binds to an exon of a target mRNA. In some embodiments, the siRNA capable of binding to a target RNA specifically binds to one target RNA. In some embodiments, the siRNA capable of binding to a target RNA is not encoded by or comprised of an intron sequence of the gene of interest. In some embodiments, the gene of interest is expressed without RNA splicing.

In some aspects, a composition of the present invention comprises a polynucleic acid construct comprising an siRNA comprising a sense strand sequence encoded by a sequence selected from SEQ ID NOs: 80-109 and SEQ ID NOs: 140-145. In some embodiments, the siRNA comprises a sense strand encoded by a sequence selected from SEQ ID NOs: 80-109 and SEQ ID NOs: 140-145, and the corresponding antisense strand encoded by a sequence selected from SEQ ID NOs: 110-139 and SEQ ID NOs: 146-151. In some aspects, a composition of the present invention comprises a polynucleic acid construct comprising an siRNA comprising a sense strand sequence encoded by a sequence selected from SEQ ID NOs: 80-92. In some embodiments, the siRNA comprises a sense strand encoded by a sequence selected from SEQ ID NOs: 80-92, and the corresponding antisense strand encoded by a sequence selected from SEQ ID NOs: 110-122. In some aspects, a composition of the present invention comprises a polynucleic acid construct comprising an siRNA comprising a sense strand sequence encoded by a sequence selected from SEQ ID NOs: 93-109. In some embodiments, the siRNA comprises a sense strand encoded by a sequence selected from SEQ ID NOs: 93-109, and the corresponding antisense strand encoded by a sequence selected from SEQ ID NOS: 123-139. In some aspects, a composition of the present invention comprises a polynucleic acid construct comprising an siRNA comprising a sense strand sequence encoded by a sequence selected from SEQ ID NOs: 140-145. In some embodiments, the siRNA comprises a sense strand encoded by a sequence selected from SEQ ID NOs: 140-145, and the corresponding antisense strand encoded by a sequence selected from SEQ ID NOs: 146-151.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic representation of construct design. T7:T7 promoter, siRNA: small interfering RNA.

FIG. 2A shows the comparison of IGF-1 mRNA construct and Compound A1 (Cpd. 1) in IGF-1 expression in HEK-293 cells while FIG. 2B shows simultaneous RNA interference of Compound A1 which comprises IL-8-targeting siRNA in an IL-8 overexpression model in HEK-293 cells. Control: IL-8 overexpression construct alone.

FIG. 3 shows dose-dependent RNA interference of Compound A1 (Cpd. 1) which comprises IL-8-targeting siRNA in an IL-8 overexpression model in HEK-293 cells.

FIG. 4A shows the modulation of IL-8 expression by Compound A2 (Cpd. 2) in THP-1 cells. Control: IL-8 overexpression construct alone.

FIG. 4B shows the IGF-1 expression of Compound A2 (Cpd. 2) in HEK-293 cells.

FIG. 5A shows the modulation of IL-8 expression by Compound A3 (Cpd. 3) in THP-1 cells. Control: IL-8 overexpression construct alone.

FIG. 5B shows the IGF-1 expression of Compound A3 (Cpd. 3) in HEK-293 cells.

FIG. 6A shows the comparison of Compound A4 (Cpd. 4) and Compound A5 (Cpd. 5) in IL-8 expression in THP-1 cells. Control: IL-8 overexpression construct alone.

FIG. 6B shows the comparison of Compound A3 (Cpd. 3) and Compound A5 (Cpd. 5) in IL-8 expression in THP1 cells. Control: IL-8 overexpression construct alone.

FIG. 7 shows the comparison of Compound A4 (Cpd. 4) and Compound A5 (Cpd. 5) in IL-8 expression in HEK-293 cells. Control: IL-8 overexpression construct alone.

FIG. 8A shows the effect of Compound A6 (Cpd. 6) in endogenous IL-1 beta (IL1b) expression in THP-1 cells. Control: LPS+dsDNA only.

FIG. 8B shows the effect of Compound A6 (Cpd. 6) in endogenous IL-1 beta (IL1b) expression in THP-1 cells. Control: LPS+dsDNA only.

FIG. 8C shows the IGF-1 expression of Compound A6 (Cpd. 6) in HEK-293 cells.

FIG. 9A shows the effect of Compound A7 (Cpd. 7) in endogenous IL-1 beta (IL1b) expression in THP-1 cells. Control: LPS+dsDNA only.

FIG. 9B shows the effect of Compound A7 (Cpd. 7) in endogenous IL-1 beta (IL1b) expression in THP-1 cells. Control: LPS+dsDNA only.

FIG. 9C shows the IGF-1 expression of Compound A7 (Cpd. 7) in HEK-293 cells.

FIG. 10A shows RNA interference of Compound A8 (Cpd. 8) which comprises TNF-α-targeting siRNA in an TNF-α overexpression model in HEK-293 cells. Control: TNF-α overexpression construct alone.

FIG. 10B shows RNA interference of Compound A8 (Cpd. 8) which comprises TNF-α-targeting siRNA in an endogenous TNF-α expression model in THP-1 cells. Control: LPS+R848 only.

FIG. 10C shows IL-4 expression of Compound A8 (Cpd. 8) in the same cell (HEK-293) culture as in FIG. 10A.

FIG. 10D shows IL-4 expression of Compound A8 (Cpd. 8) in the same cell (THP-1) culture supernatant as in FIG. 10B.

FIG. 11 depicts a phylogenetic analysis of three coronaviruses that lead to human outbreaks in the last two decades, MERS-CoV (at top), SARS-CoV-2 (middle), and SARS-CoV (bottom). The genomic sequences are publicly available (obtained from NCBI Nucleotide) and analyzed in Geneious Prime v.2019.2.3 with Tamura-Nei Genetic distance model; the tree was made with UPGMA algorithm.

FIG. 12A shows RNA interference of Compound A9 (Cpd. 9) and Compound A10 (Cpd. 10) which comprise TNF-α-targeting siRNAs in an endogenous TNF-α expression model in THP-1 cells. Control: LPS+R848 only, sc-siRNA: scrambled siRNA. Data represent means±standard error of the mean of 4 replicates. Significance (*, <0.05) was assessed by Student's t-test for siRNA activity. Significance (***, p<0.001) was assessed by one way ANOVA followed by Dunnet's multiple comparing test related to control.

FIG. 12B shows the IL-4 expression of Compound A9 (Cpd. 9) and Compound A10 (Cpd. 10) in THP-1 cells. Data represent means±standard error of the mean of 4 replicates. Significance (**, <0.01) was assessed by Student's t-test for IL-4 expression.

FIG. 13A shows RNA interference of Compound A9 (Cpd. 9) and Compound A10 (Cpd. 10) which comprise TNF-α-targeting siRNAs in an TNF-α overexpression model in HEK-293 cells. Control: TNF-α overexpression construct alone. Data represent means±standard error of the mean of 4 replicates. Significance (**, p<0.01) was assessed by one way ANOVA followed by Dunnet's multiple comparing test related to control.

FIG. 13B shows the IL-4 expression of Compound A9 (Cpd. 9) and Compound A10 (Cpd. 10) in HEK-293 cells. Data represent means±standard error of the mean of 4 replicates. Significance (***, <0.001) was assessed by Student's t-test.

FIG. 14 shows dose-dependent RNA interference of Compound A11 (Cpd. 11) which comprises ALK2-targeting siRNA in an endogenous ALK2 expression model in A549 cells and the IGF-1 expression of Compound A11 (Cpd. 11) in A549 cells. Data represent means±standard error of the mean of 4 replicates.

FIG. 15A shows dose-dependent RNA interference of Compound A12 (Cpd. 12) and Compound 13 (Cpd. 13) which comprise SOD1-targeting siRNA in an endogenous SOD1 expression model in IMR32 cells. Data represent means±standard error of the mean of 3 replicates.

FIG. 15B shows dose-dependent EPO expression of Compound A13 (Cpd. 13) in IMR32 cells. Data represent means±standard error of the mean of 4 replicates.

FIG. 15C shows dose-dependent IGF-1 expression of Compound A12 (Cpd. 12) in IMR32 cells. Data represent means±standard error of the mean of 4 replicates.

FIG. 16A shows RNA interference of Compound A14 (Cpd. 14) and Compound A15 (Cpd. 15) which comprise siRNAs targeting IL-1 beta in an IL-1 beta overexpression model in HEK-293 cells. Control: IL-1 beta overexpression construct alone. Data represent means±standard error of the mean of 4 replicates. Significance (*, <0.05) was assessed by Student's t-test. Significance (***, p<0.001) was assessed by one-way ANOVA followed by Dunnet's multiple comparing test related to control.

FIG. 16B shows the IGF-1 expression of Compound A14 (Cpd. 14) and Compound A15 (Cpd. 15) in HEK-293 cells. Data represent means±standard error of the mean of 4 replicates. Significance (***, <0.001) was assessed by Student's t-test.

FIG. 17A shows the expression of eGFP positive A549 cells transfected with pcDNA3⁺ vector containing a sequence encoding SARS CoV-2 Nucleocapsid protein tagged with eGFP.

FIG. 17B shows the expression of eGFP positive A549 cells co-transfected with pcDNA3⁺ vector containing a sequence encoding SARS CoV-2 Nucleocapsid protein tagged with eGFP and Compound B18 (Cpd. B18) comprising 3 siRNAs, one of which targets SARS CoV-2 Nucleocapsid protein.

FIG. 17C shows RNA interference of Compound B18 (Cpd. B18) which comprise siRNAs targeting SARS CoV-2 Nucleocapsid protein in A549 cells expressing SARS CoV-2 Nucleocapsid protein tagged with eGFP. Control: SARS CoV-2 Nucleocapsid protein-eGFP construct alone. Significance (***, <0.001) was assessed by Student's t-test of Compound B18 (Cpd. B18) compared to a control.

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “RNA” as used herein includes RNA which encodes an amino acid sequence (e.g., mRNA, etc.) as well as RNA which does not encode an amino acid sequence (e.g., siRNA, shRNA etc.). The RNA as used herein may be a coding RNA, i.e., an RNA which encodes an amino acid sequence. Such RNA molecules are also referred to as mRNA (messenger RNA) and are single-stranded RNA molecules. The RNA as used herein may be a non-coding RNA, i.e., an RNA which does not encode an amino acid sequence or is not translated into a protein. A non-coding RNA can include, but are not limited to, small interfering RNA (siRNA), short or small harpin RNA (shRNA), microRNA (miRNA), piwi-interacting RNA (piRNA), and long non-coding RNA (lncRNA). siRNAs as used herein may comprise a double-stranded RNA (dsRNA) region, a hairpin structure, a loop structure, or a combination thereof. In some embodiments, siRNAs as used herein may comprise at least one shRNA, at least one dsRNA region, or at least one loop structure. In some embodiments, siRNAs as used herein may be processed from a dsRNA or an shRNA. The RNA may be made by synthetic chemical and enzymatic methodology known to one of ordinary skill in the art, or by the use of recombinant technology, or may be isolated from natural sources, or by a combination thereof. The RNA may optionally comprise unnatural and naturally occurring nucleoside modifications known in the art such as e.g., N¹-Methylpseudouridine also referred herein as methylpseudouridine.

The terms “nucleic acid sequence,” “polynucleic acid sequence,” “nucleotide sequence,” and “nucleotide acid sequence” are used herein interchangeably and have the identical meaning herein and refer to preferably DNA or RNA. The terms “nucleic acid sequence,” “nucleotide sequence,” and “nucleotide acid sequence” can be used synonymously with the term “polynucleotide sequence.” In some embodiments, a nucleic acid sequence is a polymer comprising or consisting of nucleotide monomers, which are covalently linked to each other by phosphodiester-bonds of a sugar/phosphate-backbone. The term “nucleic acid sequence” also encompasses modified nucleic acid sequences, such as base-modified, sugar-modified or backbone-modified etc., DNA or RNA.

The recombinant polynucleic acid or RNA construct described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The recombinant polynucleic acid or RNA construct described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some embodiments, backbone modifications include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. In some embodiments, backbone modifications include having peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

The terms “peptide” refers to a series of amino acid residues connected one to the other, typically by peptide bonds between the α-amino and carboxyl groups of adjacent amino acid residues.

The term “target motif” or “targeting motif” as used herein can refer to any short peptide present in the newly synthesized polypeptides or proteins that are destined to any parts of cell membranes, extracellular compartments, or intracellular compartments except cytoplasm or cytosol. Intracellular compartments include, but are not limited to, intracellular organelles such as nucleus, nucleolus, endosome, proteasome, ribosome, chromatin, nuclear envelope, nuclear pore, exosome, melanosome, Golgi apparatus, peroxisome, endoplasmic reticulum (ER), lysosome, centrosome, microtubule, mitochondria, chloroplast, microfilament, intermediate filament, or plasma membrane. Other terms include, but are not limited to, signal sequence, targeting signal, localization signal, localization sequence, transit peptide, leader sequence, or leader peptide. The target motif may comprise a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS).

The term “signal peptide” also referred herein to as signaling peptide or pre-domain is a short peptide (usually 16-40 amino acids long) present at the N-terminus of newly synthesized proteins that are destined towards the secretory pathway. The signal peptide of the present invention is preferably 10-50, more preferably 11-45, even more preferably 12-45, most preferably 13-45, in particular 14-45, more particular 15-45, even more particular 16-40 amino acids long. A signal peptide according to the invention is situated at the N-terminal end of the protein of interest or at the N-terminal end of the pro-protein form of the protein of interest. A signal peptide according to the invention is usually of eukaryotic origin e.g., the signal peptide of a eukaryotic protein, preferably of mammalian origin e.g., the signal peptide of a mammalian protein, more preferably of human origin e.g., the signal peptide of a mammalian protein. In some embodiments the heterologous signal peptide and/or the homologous signal peptide to be modified is the naturally occurring signal peptide of a eukaryotic protein, preferably the naturally occurring signal peptide of a mammalian protein, more preferably the naturally occurring signal peptide of a human protein.

The term “protein” as used herein refers to molecules typically comprising one or more peptides or polypeptides. A peptide or polypeptide is typically a chain of amino acid residues, linked by peptide bonds. A peptide usually comprises between 2 and 50 amino acid residues. A polypeptide usually comprises more than 50 amino acid residues. A protein is typically folded into 3-dimensional form, which may be required for the protein to exert its biological function. The term “protein” as used herein includes a fragment of a protein and fusion proteins. In some embodiments, the protein is mammalian, e.g., of human origin, i.e., is a human protein. In some embodiments, the protein is a protein which is normally secreted from a cell, i.e., a protein which is secreted from a cell in nature, or a protein produced by a virus. In some embodiments, proteins as referred to herein are selected from the group consisting of: carboxypeptidases; cytokines; extracellular ligands and transporters, including receptors; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins. In some embodiments, the protein is a viral protein, e.g., a coronavirus protein, as described herein.

Carboxypeptidases are proteins which are protease enzymes that hydrolyze (cleave) a peptide bond at the carboxy-terminal (C-terminal) end of a protein; cytokines are proteins which are secreted and act either locally or systemically as modulators of target cell signaling via receptors on their surfaces, often involved in immunologic reactions; extracellular ligands and transporters are proteins that are secreted and act via binding to other proteins or carrying other proteins or other molecules to exert a certain biological function; extracellular matrix proteins are a collection of proteins secreted by support cells that provide structural and biochemical support to the surrounding cells; glucosidases are enzymes involved in breaking down complex carbohydrates such as starch and glycogen into their monomers; glycosyltransferases are enzymes that establish natural glycosidic linkages; growth factors are secreted proteins capable of stimulating cellular growth, proliferation, healing, and cellular differentiation either acting locally or systemically as modulators of target cell signaling via receptors on their surfaces, often involved in trophic reactions and survival or cell homeostasis signaling; growth factor binding proteins are secreted proteins binding to growth factors and thereby modulating their biological activity; heparin binding proteins are secreted proteins that interact with heparin to modulate their biological function, often in conjunction with another binding to a growth factor or hormone; hormones are members of a class of signaling molecules produced by glands in multicellular organisms that are secreted and transported by the circulatory system to target distant organs to regulate physiology and behavior via binding to specific receptors on their target cells; hydrolases are a class of enzymes that biochemically catalyze molecule cleavage by utilizing water to break chemical bonds, resulting in a division of a larger molecule to smaller molecules; immunoglobulins are large, Y-shaped secreted proteins produced mainly by plasma cells that are used by the immune system to neutralize pathogens such as pathogenic bacteria and viruses; isomerases are a general class of enzymes that convert a molecule from one isomer to another, thereby facilitating intramolecular rearrangements in which bonds are broken and formed; kinases are enzymes catalyzing the transfer of phosphate groups from high-energy, phosphate-donating molecules to specific substrates; lyases are enzymes catalyzing the breaking of various chemical bonds by means other than hydrolysis and oxidation, often forming a new double bond or a new ring structure; metalloenzyme inhibitors cellular inhibitors of the Matrix metalloproteases (MMPs); metalloproteases are protease enzymes whose catalytic mechanism involves a metal ion; milk proteins are proteins secreted into milk; neuroactive proteins are secreted proteins that act either locally or via distances to support neuronal function, survival and physiology; proteases (also called peptidases or proteinases) are enzymes that perform proteolysis by hydrolysis of peptide bonds; protease inhibitors are proteins that inhibit the function of proteases; protein phosphatases are enzymes that remove phosphate groups from phosphorylated amino acid residues of their substrate protein; esterases are enzymes that split esters into an acid and an alcohol in a chemical reaction with water at an amino acid residue; transferases are a class of enzymes that catalyze the transfer of specific functional groups (e.g., a methyl or glycosyl group) from one molecule (called the donor) to another (called the acceptor); vasoactive proteins are secreted proteins that biologically affect function of blood vessels. Carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins as referred to herein can be found in the UniProt database.

In some embodiments, proteins as referred to herein are, e.g., cytokines, proteins that are secreted and act either locally or systemically as modulators of target cell signaling via receptors on their surfaces, often involved in immunologic reactions, other host proteins involved in viral infection, and virus proteins. Nucleotide and amino acid sequences of proteins useful in the context of the present invention, including proteins that are encoded by a gene of interest, are known in the art and available in the literature, e.g., in the UniProt database.

The terms “fragment,” or “fragment of a sequence” which have the identical meaning herein is a shorter portion of a full-length sequence of e.g., a nucleic acid molecule like DNA or RNA or a protein. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 5%, usually at least 20%, preferably at least 30%, more preferably at least 40%, more preferably at least 50%, even more preferably at least 60%, even more preferably at least 70%, and most preferably at least 80% of the total (i.e., full-length) molecule, from which the fragment is derived.

The term “vector” or “expression vector” as used herein refers to naturally occurring or synthetically generated constructs for uptake, proliferation, expression or transmission of nucleic acids in a cell, e.g., plasmids, minicircles, phagemids, cosmids, artificial chromosomes/mini-chromosomes, bacteriophages, viruses such as baculovirus, retrovirus, adenovirus, adeno-associated virus, herpes simplex virus, bacteriophages. Vectors can either integrate into the genome of the host cell or remain as autonomously replicating construct within the host cell. Methods used to construct vectors are well known to a person skilled in the art and described in various publications. In particular techniques for constructing suitable vectors, including a description of the functional and regulatory components such as promoters, enhancers, termination and polyadenylation signals, selection markers, origins of replication, and splicing signals, are known to the person skilled in the art. The eukaryotic expression vectors will typically contain also prokaryotic sequences that facilitate the propagation of the vector in bacteria such as an origin of replication and antibiotic resistance genes for selection in bacteria which might be removed before transfection of eukaryotic cells. A variety of eukaryotic expression vectors, containing a cloning site into which a polynucleotide can be operably linked, are well known in the art and some are commercially available from companies such as Agilent Technologies, Santa Clara, Calif.; Invitrogen, Carlsbad, Calif.; Promega, Madison, Wis. or Invivogen, San Diego, Calif.

The term “transcription unit,” “expression unit,” or “expression cassette” as used herein refers a region within a vector, construct or polynucleotide sequence that contains one or more genes to be transcribed, wherein the genes contained within the segment are operably linked to each other. They are transcribed from a single promoter and transcription is terminated by at least one polyadenylation signal. As a result, the different genes are at least transcriptionally linked. More than one protein or product can be transcribed and expressed from each transcription unit (multicistronic transcription unit). Each transcription unit will comprise the regulatory elements necessary for the transcription and translation of any of the selected sequence that are contained within the unit. And each transcription unit may contain the same or different regulatory elements. For example, each transcription unit may contain the same terminator. IRES element or introns may be used for the functional linking of the genes within a transcription unit. A vector or polynucleotide sequence may contain more than one transcription unit.

The term “skeletal muscle injury” as used herein refers to any injuries and ruptures of skeletal muscle, preferably ruptures of skeletal muscle, induced by eccentric muscle contractions, elongations and muscle overload. In principle any skeletal muscle can be affected by such injury or rupture. Preferably skeletal muscle injury are injuries and ruptures of skeletal muscle wherein the skeletal muscles are selected from the muscle groups of the head, the neck, the thorax, the back, the abdomen, the pelvis, the arms, the legs and the hip.

More preferably skeletal muscle injury are injuries and ruptures wherein the skeletal muscles are selected from the group consisting of plantaris, temporal, papillary, pectoralis major, tibialis posterior, tibialis anterior, gastrocnemius, coracobrachialis, diaphragma, palmaris longus, rectus abdominis, external anal sphincter, internal anal sphincter, subscapularis, biceps, triceps, quadriceps, calf, groin, hamstring, deltoid, teres major, rotator cuff supraspinatus, rotator cuff infraspinatus, rotator cuff teres minor, rotator cuff subscapularis, rectus femoralis, rectus abdominis, abdominal external oblique, masseter, trapezius, latissimus, pectoralis, erector spinae, iliocostalis, longissimus, spinalis, latissimus dorsi, transversospinales, semispinalis dorsi, semispinalis cervices, semispinalis capitis, multifidus, rotatores, interspinales, intertransversarii, splenius capitis, splenius cervices, intercostals, subcostales, transversus thoracis, levatores costarum, serratus posterior inferior, serratus posterior superior, Transversus abdominis, rectus abdominis, pyramidalis, cremaster, quadratus lumborum, external oblique, internal oblique. Even more preferably skeletal muscle injury are injuries and ruptures wherein the skeletal muscles are selected from the group consisting of plantaris, temporal, papillary, pectoralis major, tibialis posterior, tibialis anterior, gastrocnemius, coracobrachialis, diaphragma, palmaris longus, rectus abdominis, external anal sphincter, internal anal sphincter, subscapularis, biceps, triceps, quadriceps, calf, groin, hamstring, deltoid, teres major, rotator cuff supraspinatus, rotator cuff infraspinatus, rotator cuff teres minor, rotator cuff subscapularis, rectus femoralis, rectus abdominis, abdominal external oblique, masseter, trapezius, latissimus, pectoralis.

Preferably any injuries and ruptures of skeletal muscle, preferably ruptures of skeletal muscle, induced by eccentric muscle contraction, elongation or muscle overload are treated by the method of the present invention.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The terms “pharmaceutical composition” and “pharmaceutical formulation” (or “formulation”) are used interchangeably and denote a mixture or solution comprising a therapeutically effective amount of an active pharmaceutical ingredient together with one or more pharmaceutically acceptable excipients to be administered to a subject, e.g., a human in need thereof.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The term “recombinant polynucleic acid” or “recombinant RNA” can refer to a polynucleic acid or RNA that are not naturally occurring and are synthesized or manipulated in vitro. A recombinant polynucleic acid or RNA can be synthesized in a laboratory and can be prepared by using recombinant DNA or RNA technology by using enzymatic modification of DNA or RNA, such as enzymatic restriction digestion, ligation, and cloning. A recombinant polynucleic acid can be transcribed in vitro to produce a messenger RNA (mRNA) and the recombinant mRNA can be isolated, purified, and used for transfection. A recombinant polynucleic acid or RNA used herein can encode a protein, polypeptide, a target motif, a signal peptide, and/or a non-coding RNA such as small interfering RNA (siRNA). Under suitable conditions, a recombinant polynucleic acid or RNA can be incorporated into a cell and expressed within the cell.

The term “expression” of a polynucleic acid, gene, DNA, or RNA, as used herein, can refer to transcription and/or translation of the polynucleic acid, gene, DNA, or RNA. The term “modulating,” “increasing,” “upregulating,” “decreasing,” or “downregulating” the expression of a polynucleic acid, gene such as a gene of interest, DNA, or RNA such as a target mRNA, as used herein, can refer to modulating, increasing, upregulating, decreasing, downregulating the level of protein encoded by a polynucleic acid, gene such as a gene of interest, DNA, or RNA such as a target mRNA by affecting transcription and/or translation of the polynucleic acid, gene such as a gene of interest, DNA, or RNA such as a target mRNA. The term “inhibiting” the expression of a polynucleic acid, gene such as a gene of interest, DNA, or RNA such as a target mRNA can refer to affect transcription and/or translation of the polynucleic acid, gene such as a gene of interest, DNA, or RNA such as a target mRNA such that the level of protein encoded by the polynucleic acid, gene such as a gene of interest, DNA, or RNA such as a target mRNA is reduced or abolished.

The term “operably linked” can refer to a functional relationship between two or more nucleic acid sequences, e.g., a functional relationship of a transcriptional regulatory or signal sequence to a transcribed sequence. For example, a target motif or a nucleic acid encoding a target motif is operably linked to a coding sequence if it is expressed as a preprotein that participates in targeting the polypeptide encoded by the coding sequence to a cell membrane, intracellular, or an extracellular compartment. For example, a signal peptide or a nucleic acid encoding a signal peptide is operably linked to a coding sequence if it is expressed as a preprotein that participates in the secretion of the polypeptide encoded by the coding sequence. For example, a promoter is operably linked if it stimulates or modulates the transcription of the coding sequence.

The term “Kozak sequence,” “Kozak consensus sequence,” or “Kozak consensus” can refer to a nucleic acid sequence motif that functions as the protein translation initiation site. Kozak sequences are described at length in the literature, e.g., by Kozak, M., Gene 299(1-2):1-34, incorporated herein by reference herein in its entirety.

### Construct Design

The present invention disclosed herein refers to a composition comprising a polynucleic acid or RNA construct to express (i) siRNAs capable of binding to one or more target RNA (e.g., mRNA) and (ii) one or more genes of interest from a single RNA transcript. The present invention provides a means to express (i) siRNAs capable of binding to one or more target mRNA and (ii) one or more protein of interest simultaneously from a single RNA transcript. The present invention provides a means to modulate expression of two or more genes simultaneously. In some embodiments, siRNA capable of binding to a target mRNA in the composition downregulates the expression of the target mRNA while simultaneously the gene of interest is expressed or overexpressed to increase the level of protein encoded by the gene of interest. In some embodiments, the recombinant polynucleic acid or RNA construct of the present invention comprises (i) siRNAs that can target multiple mRNAs and multiple genes of interest, (ii) multiple copies of siRNAs that can target one mRNA and multiple copies of the same gene of interest, or (iii) combination of the (i) and (ii). In some embodiments, the recombinant polynucleic acid or RNA construct of the present invention comprise siRNAs that target multiple mRNAs and multiple copies of the same gene of interest. In some embodiments, the recombinant polynucleic acid or RNA construct of the present invention comprise multiple copies of siRNAs that can target one mRNA and multiple genes of interest.

In some aspects, provided herein, is a composition comprising a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest. In some embodiments, the at least one nucleic acid sequence encoding or comprising the siRNA capable of binding to the target and the at least one nucleic acid sequence encoding the gene of interest are separated. In some embodiments, the at least one nucleic acid sequence encoding or comprising the siRNA capable of binding to the target and the at least one nucleic acid sequence encoding the gene of interest are separated by a nucleic acid sequence. In some embodiments the separating nucleic acid sequence encodes or comprises a linker. In some embodiments, the at least one nucleic acid sequence encoding or comprising the siRNA capable of binding to the target and the at least one nucleic acid sequence encoding the gene of interest are arranged in tandem. For example, the at least one nucleic acid sequence encoding or comprising the siRNA capable of binding to the target RNA is not inserted within the at least one nucleic acid sequence encoding the gene of interest. For example, the at least one nucleic acid sequence encoding or comprising the siRNA capable of binding to the target RNA is not inserted within an intronic sequence of the at least one nucleic acid sequence encoding the gene of interest. In some embodiments, the siRNA does not affect the expression of the gene of interest. In some embodiments, the siRNA does not reduce the expression of the gene of interest. In some embodiments, the composition comprising a recombinant polynucleic acid construct further comprises or encodes a linker. In some embodiments, the nucleic acid sequence encoding or comprising the linker connects (i) and (ii). In some embodiments, the nucleic acid sequence encoding or comprising the linker connects the at least one nucleic acid sequence encoding or comprising a small interfering RNA (siRNA) capable of binding to a target messenger RNA (mRNA) and the at least one nucleic acid sequence encoding a gene of interest. In some embodiments, the linker comprises a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising

(SEQ ID NO: 24)
AACAAAGCACCAGTGGTCTAGTGGTAGAATAGTACCCTGCCACGGTA
CAGACCCGGGTTCGATTCCCGGCTGGTGCA.

In some aspects, provided herein, is a composition comprising a recombinant RNA construct comprising: (i) a small interfering RNA (siRNA) capable of binding to a target messenger RNA (mRNA); and (ii) an mRNA encoding a gene of interest; wherein the target mRNA is different from the mRNA encoding the gene of interest.

In some embodiments, (i) and (ii) may be comprised in 5′ to 3′ direction. In some embodiments, (i) and (ii) may not be comprised in 5′ to 3′ direction. In some embodiments (i) and (ii) may be comprised in 3′ to 5′ direction. In some embodiments, (i) and (ii) may not be comprised or present in a sequential manner. In some embodiments, (i) and (ii) may be comprised or present in a sequential manner. In some aspects, the at least one nucleic acid sequence encoding or comprising a small interfering RNA (siRNA) capable of binding to a target RNA (i) and the at least one nucleic acid sequence encoding a gene of interest (ii) are comprised or present in a sequential manner. In some aspects, the nucleic acid sequence encoding or comprising a small interfering RNA (siRNA) capable of binding to a target RNA (i) is upstream of the at least one nucleic acid sequence encoding a gene of interest (ii). In some aspects, the nucleic acid sequence encoding or comprising a small interfering RNA (siRNA) capable of binding to a target RNA (i) is downstream of the at least one nucleic acid sequence encoding a gene of interest (ii). In some aspects, the nucleic acid sequence encoding or comprising a small interfering RNA (siRNA) capable of binding to a target RNA (i) is upstream or downstream of the at least one nucleic acid sequence encoding a gene of interest (ii). In some aspects, the siRNA capable of binding to a target RNA binds to an exon of a target mRNA. In some aspects, the siRNA capable of binding to a target RNA specifically binds to one target RNA. In some aspects, the siRNA capable of binding to a target RNA is not encoded by or comprised of an intron sequence of the gene of interest. In some aspects, the gene of interest is expressed without RNA splicing. In some embodiments, (i) and (ii) may be separated. In some embodiments, (i) and (ii) may be arranged in tandem. In some embodiments, the siRNA capable of binding to the target RNA and the mRNA encoding the gene of interest are separated. In some embodiments, the siRNA capable of binding to the target RNA and the mRNA encoding the gene of interest are arranged in tandem. For example, the siRNA capable of binding to the target RNA is located either upstream or downstream of the mRNA encoding the gene of interest in the composition.

In some embodiments, the expression of the gene of interest is increased when the composition comprises a nucleic acid sequence encoding or comprising a small interfering RNA (siRNA) capable of binding to a target RNA downstream of (or 3′ to) a nucleic acid sequence encoding a gene of interest, compared to the expression of the gene of interest from a composition comprising a nucleic acid sequence encoding or comprising an siRNA capable of binding to a target RNA upstream of (or 5′ to) a nucleic acid sequence encoding a gene of interest. In some embodiments, the expression of the gene of interest is increased when the composition comprises a nucleic acid sequence encoding or comprising a small interfering RNA (siRNA) capable of binding to a target RNA and a nucleic acid sequence encoding a gene of interest in 3′ to 5′ direction, compared to the expression of the gene of interest from a composition comprising a nucleic acid sequence encoding or comprising an siRNA capable of binding to a target RNA and a nucleic acid sequence encoding a gene of interest in 5′ to 3′ direction.

As described herein, in some embodiments, in a composition comprising a recombinant polynucleic acid construct, the at least one nucleic acid sequence encoding or comprising the at least one small interfering RNA (siRNA) capable of binding to a target RNA and the at least one nucleic acid sequence encoding a gene of interest are comprised in a sequential manner. In some embodiments, in a composition comprising a recombinant polynucleic acid construct, the at least one nucleic acid sequence encoding or comprising the at least one small interfering RNA (siRNA) capable of binding to a target RNA and the at least one nucleic acid sequence encoding a gene of interest are present in a sequential manner. In some embodiments, the composition comprises the at least one nucleic acid sequence encoding or comprising two or more, preferably 2 to 10, more preferably 2 to 6, small interfering RNAs (siRNAs) capable of binding to a target RNA and the at least one nucleic acid sequence encoding a gene of interest in a sequential manner. In some embodiments, the expression of the gene of interest is decreased when the composition comprises a nucleic acid sequence encoding or comprising two or more, preferably 2 to 10, more preferably 2 to 6, siRNAs capable of binding to a target RNA and a nucleic acid sequence encoding a gene of interest in 5′ to 3′ direction, compared to the expression of the gene of interest from a composition comprising a nucleic acid sequence encoding or comprising two or more siRNAs capable of binding to a target RNA and a nucleic acid sequence encoding a gene of interest in 3′ to 5′ direction. In some embodiments, the expression of the gene of interest is decreased when the composition comprises a nucleic acid sequence encoding or comprising two or more, preferably 2 to 10, more preferably 2 to 6, siRNAs capable of binding to a target RNA upstream of (or 5′ to) the nucleic acid sequence encoding a gene of interest, compared to the expression of the gene of interest from a composition comprising a nucleic acid sequence encoding or comprising two or more siRNAs capable of binding to a target RNA downstream of (or 3′ to) a nucleic acid sequence encoding a gene of interest. In some embodiments, the expression of the gene of interest is decreased when the sequential manner comprises the at least one nucleic acid sequence encoding a gene of interest positioned downstream of (3′ to), the at least one nucleic acid sequence encoding or comprising the two or more, preferably 2 to 10, more preferably 2 to 6, siRNAs, relative to the expression of the gene of interest when the sequential manner comprises the at least one nucleic acid sequence encoding a gene of interest positioned upstream of (5′ to), the at least one nucleic acid sequence encoding or comprising the two or more siRNAs.

In some embodiments, the expression of the gene of interest is increased when the composition comprises a nucleic acid sequence encoding or comprising two or more, preferably 2 to 10, more preferably 2 to 6, siRNAs capable of binding to a target RNA and a nucleic acid sequence encoding a gene of interest in 3′ to 5′ direction, compared to the expression of the gene of interest from a composition comprising a nucleic acid sequence encoding or comprising two or more siRNAs capable of binding to a target RNA and a nucleic acid sequence encoding a gene of interest in 5′ to 3′ direction. In some embodiments, the expression of the gene of interest is increased when the composition comprises a nucleic acid sequence encoding or comprising two or more, preferably 2 to 10, more preferably 2 to 6, siRNAs capable of binding to a target RNA downstream of (or 5′ to) the nucleic acid sequence encoding a gene of interest, compared to the expression of the gene of interest from a composition comprising a nucleic acid sequence encoding or comprising two or more siRNAs capable of binding to a target RNA upstream of (or 3′ to) a nucleic acid sequence encoding a gene of interest. In some embodiments, the expression of the gene of interest is increased when the sequential manner comprises the at least one nucleic acid sequence encoding a gene of interest positioned upstream of (5′ to), the at least one nucleic acid sequence encoding or comprising the two or more, preferably 2 to 10, more preferably 2 to 6, siRNA, relative to the expression of the gene of interest when the sequential manner comprises the at least one nucleic acid sequence encoding a gene of interest positioned downstream of (3′ to), the at least one nucleic acid sequence encoding or comprising the two or more siRNA.

In some embodiments, the downregulation of the target RNA is enhanced when the composition comprises a nucleic acid sequence encoding or comprising two or more, preferably 2 to 10, more preferably 2 to 6, small interfering RNAs (siRNAs) capable of binding to a target RNA downstream of (or 3′ to) a nucleic acid sequence encoding a gene of interest, compared to the downregulation of the target RNA from a composition comprising a nucleic acid sequence encoding or comprising two or more siRNAs capable of binding to a target RNA upstream of (or 5′ to) a nucleic acid sequence encoding a gene of interest. In some embodiments, the downregulation of the target RNA is enhanced when the composition comprises a nucleic acid sequence encoding or comprising two or more, preferably 2 to 10, more preferably 2 to 6, siRNAs capable of binding to a target RNA and a nucleic acid sequence encoding a gene of interest in 3′ to 5′ direction, compared to the downregulation of the target RNA from a composition comprising a nucleic acid sequences encoding or comprising two or more siRNA capable of binding to a target RNA and a nucleic acid sequence encoding a gene of interest in 5′ to 3′ direction. In some embodiments, the downregulation of the target RNA is enhanced when the sequential manner comprises the at least one nucleic acid sequence encoding or comprising two or more, preferably 2 to 10, more preferably 2 to 6, siRNAs positioned downstream of (3′ to), the at least one nucleic acid sequence encoding the gene of interest, relative to the downregulation of the target RNA when the sequential manner comprises the at least one nucleic acid sequence encoding or comprising two or more siRNAs positioned upstream of (5′ to), the at least one nucleic acid sequence encoding the gene of interest.

In some embodiments, the downregulation of the target RNA is reduced when the composition comprises a nucleic acid sequence encoding or comprising two or more, preferably 2 to 10, more preferably 2 to 6, small interfering RNAs (siRNAs) capable of binding to a target RNA upstream of (or 5′ to) a nucleic acid sequence encoding a gene of interest, compared to the downregulation of the target RNA from a composition comprising a nucleic acid sequence encoding or comprising two or more siRNAs capable of binding to a target RNA downstream of (or 3′ to) a nucleic acid sequence encoding a gene of interest. In some embodiments, the downregulation of the target RNA is reduced when the composition comprises a nucleic acid sequence encoding or comprising two or more, preferably 2 to 10, more preferably 2 to 6, siRNAs capable of binding to a target RNA and a nucleic acid sequence encoding a gene of interest in 5′ to 3′ direction, compared to the downregulation of the target RNA from a composition comprising a nucleic acid sequence encoding or comprising two or more siRNAs capable of binding to a target RNA and a nucleic acid sequence encoding a gene of interest in 3′ to 5′ direction. In some embodiments, the downregulation of the target RNA is reduced when the sequential manner comprises the at least one nucleic acid sequence encoding or comprising two or more, preferably 2 to 10, more preferably 2 to 6, siRNAs positioned upstream of (5′ to), the at least one nucleic acid sequence encoding the gene of interest, relative to the downregulation of the target RNA when the sequential manner comprises the at least one nucleic acid sequence encoding or comprising two or more siRNAs positioned downstream of (3′ to), the at least one nucleic acid sequence encoding the gene of interest.

In some embodiments, the expression of the gene of interest is increased, and the downregulation of the target RNA is enhanced, when the sequential manner comprises the at least one nucleic acid sequence encoding a gene of interest positioned upstream of (5′ to), the at least one nucleic acid sequence encoding or comprising two or more, preferably 2 to 10, more preferably 2 to 6, siRNAs, relative to the expression of the gene of interest when the sequential manner comprises the at least one nucleic acid sequence encoding a gene of interest positioned downstream of (3′ to), the at least one nucleic acid sequence encoding or comprising two or more siRNAs.

In some embodiments, the relative increase in the expression of the gene of interest is about 2-fold to about 30-fold. In some embodiments, the relative increase in the expression of the gene of interest is about 2 fold to about 30 fold. In some embodiments, the relative increase in the expression of the gene of interest is about 2 fold to about 5 fold, about 2 fold to about 10 fold, about 2 fold to about 15 fold, about 2 fold to about 17 fold, about 2 fold to about 18 fold, about 2 fold to about 19 fold, about 2 fold to about 20 fold, about 2 fold to about 21 fold, about 2 fold to about 22 fold, about 2 fold to about 25 fold, about 2 fold to about 30 fold, about 5 fold to about 10 fold, about 5 fold to about 15 fold, about 5 fold to about 17 fold, about 5 fold to about 18 fold, about 5 fold to about 19 fold, about 5 fold to about 20 fold, about 5 fold to about 21 fold, about 5 fold to about 22 fold, about 5 fold to about 25 fold, about 5 fold to about 30 fold, about 10 fold to about 15 fold, about 10 fold to about 17 fold, about 10 fold to about 18 fold, about 10 fold to about 19 fold, about 10 fold to about 20 fold, about 10 fold to about 21 fold, about 10 fold to about 22 fold, about 10 fold to about 25 fold, about 10 fold to about 30 fold, about 15 fold to about 17 fold, about 15 fold to about 18 fold, about 15 fold to about 19 fold, about 15 fold to about 20 fold, about 15 fold to about 21 fold, about 15 fold to about 22 fold, about 15 fold to about 25 fold, about 15 fold to about 30 fold, about 17 fold to about 18 fold, about 17 fold to about 19 fold, about 17 fold to about 20 fold, about 17 fold to about 21 fold, about 17 fold to about 22 fold, about 17 fold to about 25 fold, about 17 fold to about 30 fold, about 18 fold to about 19 fold, about 18 fold to about 20 fold, about 18 fold to about 21 fold, about 18 fold to about 22 fold, about 18 fold to about 25 fold, about 18 fold to about 30 fold, about 19 fold to about 20 fold, about 19 fold to about 21 fold, about 19 fold to about 22 fold, about 19 fold to about 25 fold, about 19 fold to about 30 fold, about 20 fold to about 21 fold, about 20 fold to about 22 fold, about 20 fold to about 25 fold, about 20 fold to about 30 fold, about 21 fold to about 22 fold, about 21 fold to about 25 fold, about 21 fold to about 30 fold, about 22 fold to about 25 fold, about 22 fold to about 30 fold, or about 25 fold to about 30 fold. In some embodiments, the relative increase in the expression of the gene of interest is about 2 fold, about 5 fold, about 10 fold, about 15 fold, about 17 fold, about 18 fold, about 19 fold, about 20 fold, about 21 fold, about 22 fold, about 25 fold, or about 30 fold. In some embodiments, the relative increase in the expression of the gene of interest is at least about 2 fold, about 5 fold, about 10 fold, about 15 fold, about 17 fold, about 18 fold, about 19 fold, about 20 fold, about 21 fold, about 22 fold, or about 25 fold. In some embodiments, the relative increase in the expression of the gene of interest is at most about 5 fold, about 10 fold, about 15 fold, about 17 fold, about 18 fold, about 19 fold, about 20 fold, about 21 fold, about 22 fold, about 25 fold, or about 30 fold.

In embodiments, the relative enhancement of target RNA downregulation is about 1.1 fold to about 5 fold. In embodiments, the relative enhancement of target RNA downregulation is about 1.1 fold to about 1.75 fold, about 1.1 fold to about 2 fold, about 1.1 fold to about 2.25 fold, about 1.1 fold to about 2.5 fold, about 1.1 fold to about 3 fold, about 1.1 fold to about 3.5 fold, about 1.1 fold to about 4 fold, about 1.1 fold to about 4.5 fold, about 1.1 fold to about 5 fold, about 1.5 fold to about 1.75 fold, about 1.5 fold to about 2 fold, about 1.5 fold to about 2.25 fold, about 1.5 fold to about 2.5 fold, about 1.5 fold to about 3 fold, about 1.5 fold to about 3.5 fold, about 1.5 fold to about 4 fold, about 1.5 fold to about 4.5 fold, about 1.5 fold to about 5 fold, about 1.75 fold to about 2 fold, about 1.75 fold to about 2.25 fold, about 1.75 fold to about 2.5 fold, about 1.75 fold to about 3 fold, about 1.75 fold to about 3.5 fold, about 1.75 fold to about 4 fold, about 1.75 fold to about 4.5 fold, about 1.75 fold to about 5 fold, about 2 fold to about 2.25 fold, about 2 fold to about 2.5 fold, about 2 fold to about 3 fold, about 2 fold to about 3.5 fold, about 2 fold to about 4 fold, about 2 fold to about 4.5 fold, about 2 fold to about 5 fold, about 2.25 fold to about 2.5 fold, about 2.25 fold to about 3 fold, about 2.25 fold to about 3.5 fold, about 2.25 fold to about 4 fold, about 2.25 fold to about 4.5 fold, about 2.25 fold to about 5 fold, about 2.5 fold to about 3 fold, about 2.5 fold to about 3.5 fold, about 2.5 fold to about 4 fold, about 2.5 fold to about 4.5 fold, about 2.5 fold to about 5 fold, about 3 fold to about 3.5 fold, about 3 fold to about 4 fold, about 3 fold to about 4.5 fold, about 3 fold to about 5 fold, about 3.5 fold to about 4 fold, about 3.5 fold to about 4.5 fold, about 3.5 fold to about 5 fold, about 4 fold to about 4.5 fold, about 4 fold to about 5 fold, or about 4.5 fold to about 5 fold. In embodiments, the relative enhancement of target RNA downregulation is about 1.5 fold, about 1.75 fold, about 2 fold, about 2.25 fold, about 2.5 fold, about 3 fold, about 3.5 fold, about 4 fold, about 4.5 fold, or about 5 fold. In embodiments, the relative enhancement of target RNA downregulation is at least about 1.5 fold, about 1.75 fold, about 2 fold, about 2.25 fold, about 2.5 fold, about 3 fold, about 3.5 fold, about 4 fold, or about 4.5 fold. In embodiments, the relative enhancement of target RNA downregulation is at most about 1.75 fold, about 2 fold, about 2.25 fold, about 2.5 fold, about 3 fold, about 3.5 fold, about 4 fold, about 4.5 fold, or about 5 fold.

In some embodiments, the expression of the gene of interest is increased by about 2-fold to about 30-fold, and the downregulation of the target RNA is enhanced by about 1.1 fold to about 5 fold, when the sequential manner comprises the at least one nucleic acid sequence encoding a gene of interest positioned upstream of (5′ to), the at least one nucleic acid sequence encoding or comprising the two or more siRNAs, relative to the expression of the gene of interest when the sequential manner comprises the at least one nucleic acid sequence encoding a gene of interest positioned downstream of (3′ to), the at least one nucleic acid sequence encoding or comprising the two or more siRNAs.

In some embodiments, the composition comprising a recombinant RNA construct further encodes or comprises a linker. In some embodiments, the nucleic acid sequence encoding or comprising the linker connects (i) and (ii). In some embodiments, the nucleic acid sequence encoding or comprising the linker connects the small interfering RNA (siRNA) capable of binding to a target messenger RNA (mRNA) and the mRNA encoding a gene of interest. In some embodiments, the linker comprises a tRNA linker. In some embodiments, the tRNA linker may comprise a nucleic acid sequence comprising

(SEQ ID NO: 24)
AACAAAGCACCAGTGGTCTAGTGGTAGAATAGTACCCTGCCACGGTA
CAGACCCGGGTTCGATTCCCGGCTGGTGCA.

In some embodiments, the recombinant polynucleic acid construct encodes a linker. In some embodiments, the encoded linker is a 2A peptide linker. In some aspects, the linker encoded or comprised by the recombinant nucleic acid construct is at least 6 nucleic acid residues in length. In some aspects, the linker encoded or comprised by the recombinant polynucleic acid construct is at least 7, at least 8, at least 9, at least 10, at least 11, at least 12, at least 13, at least 14, at least 15, at least 20, at least 25, at least 30, at least 35, or at least 40, nucleic acid residues in length. In some aspects, the nucleic acid sequence encoding or comprising the linker is up to 50 nucleic acid residues in length. In some aspects, the nucleic acid sequence encoding or comprising the linker is up to 80 nucleic acid residues in length. In some aspects, the nucleic acid sequence encoding or comprising the linker is up to 10, up to 15, up to 20, up to 25, up to 30, up to 35, up to 40, up to 45, up to 50, up to 55, up to 60, up to 65, up to 70, or up to 75 nucleic acid residues in length. In some aspects, the nucleic acid sequence encoding or comprising the linker is about 6 nucleic acid residues in length to about 50 nucleic acid residues in length. In some aspects, the nucleic acid sequence encoding or comprising the linker is about 6 nucleic acid residues in length to about 80 nucleic acid residues in length. In some aspects, the linker is about 6 nucleic acid residues in length to about 8 nucleic acid residues in length, about 6 nucleic acid residues in length to about 10 nucleic acid residues in length, about 6 nucleic acid residues in length to about 12 nucleic acid residues in length, about 6 nucleic acid residues in length to about 15 nucleic acid residues in length, about 6 nucleic acid residues in length to about 20 nucleic acid residues in length, about 6 nucleic acid residues in length to about 25 nucleic acid residues in length, about 6 nucleic acid residues in length to about 30 nucleic acid residues in length, about 6 nucleic acid residues in length to about 35 nucleic acid residues in length, about 6 nucleic acid residues in length to about 40 nucleic acid residues in length, about 6 nucleic acid residues in length to about 45 nucleic acid residues in length, about 6 nucleic acid residues in length to about 50 nucleic acid residues in length, about 6 nucleic acid residues in length to about 60 nucleic acid residues in length, about 6 nucleic acid residues in length to about 70 nucleic acid residues in length, about 6 nucleic acid residues in length to about 80 nucleic acid residues in length, about 8 nucleic acid residues in length to about 10 nucleic acid residues in length, about 8 nucleic acid residues in length to about 12 nucleic acid residues in length, about 8 nucleic acid residues in length to about 15 nucleic acid residues in length, about 8 nucleic acid residues in length to about 20 nucleic acid residues in length, about 8 nucleic acid residues in length to about 25 nucleic acid residues in length, about 8 nucleic acid residues in length to about 30 nucleic acid residues in length, about 8 nucleic acid residues in length to about 35 nucleic acid residues in length, about 8 nucleic acid residues in length to about 40 nucleic acid residues in length, about 8 nucleic acid residues in length to about 45 nucleic acid residues in length, about 8 nucleic acid residues in length to about 50 nucleic acid residues in length, about 10 nucleic acid residues in length to about 12 nucleic acid residues in length, about 10 nucleic acid residues in length to about 15 nucleic acid residues in length, about 10 nucleic acid residues in length to about 20 nucleic acid residues in length, about 10 nucleic acid residues in length to about 25 nucleic acid residues in length, about 10 nucleic acid residues in length to about 30 nucleic acid residues in length, about 10 nucleic acid residues in length to about 35 nucleic acid residues in length, about 10 nucleic acid residues in length to about 40 nucleic acid residues in length, about 10 nucleic acid residues in length to about 45 nucleic acid residues in length, about 10 nucleic acid residues in length to about 50 nucleic acid residues in length, about 12 nucleic acid residues in length to about 15 nucleic acid residues in length, about 12 nucleic acid residues in length to about 20 nucleic acid residues in length, about 12 nucleic acid residues in length to about 25 nucleic acid residues in length, about 12 nucleic acid residues in length to about 30 nucleic acid residues in length, about 12 nucleic acid residues in length to about 35 nucleic acid residues in length, about 12 nucleic acid residues in length to about 40 nucleic acid residues in length, about 12 nucleic acid residues in length to about 45 nucleic acid residues in length, about 12 nucleic acid residues in length to about 50 nucleic acid residues in length, about 15 nucleic acid residues in length to about 20 nucleic acid residues in length, about 15 nucleic acid residues in length to about 25 nucleic acid residues in length, about 15 nucleic acid residues in length to about 30 nucleic acid residues in length, about 15 nucleic acid residues in length to about 35 nucleic acid residues in length, about 15 nucleic acid residues in length to about 40 nucleic acid residues in length, about 15 nucleic acid residues in length to about 45 nucleic acid residues in length, about 15 nucleic acid residues in length to about 50 nucleic acid residues in length, about 20 nucleic acid residues in length to about 25 nucleic acid residues in length, about 20 nucleic acid residues in length to about 30 nucleic acid residues in length, about 20 nucleic acid residues in length to about 35 nucleic acid residues in length, about 20 nucleic acid residues in length to about 40 nucleic acid residues in length, about 20 nucleic acid residues in length to about 45 nucleic acid residues in length, about 20 nucleic acid residues in length to about 50 nucleic acid residues in length, about 25 nucleic acid residues in length to about 30 nucleic acid residues in length, about 25 nucleic acid residues in length to about 35 nucleic acid residues in length, about 25 nucleic acid residues in length to about 40 nucleic acid residues in length, about 25 nucleic acid residues in length to about 45 nucleic acid residues in length, about 25 nucleic acid residues in length to about 50 nucleic acid residues in length, about 30 nucleic acid residues in length to about 35 nucleic acid residues in length, about 30 nucleic acid residues in length to about 40 nucleic acid residues in length, about 30 nucleic acid residues in length to about 45 nucleic acid residues in length, about 30 nucleic acid residues in length to about 50 nucleic acid residues in length, about 35 nucleic acid residues in length to about 40 nucleic acid residues in length, about 35 nucleic acid residues in length to about 45 nucleic acid residues in length, about 35 nucleic acid residues in length to about 50 nucleic acid residues in length, about 40 nucleic acid residues in length to about 45 nucleic acid residues in length, about 40 nucleic acid residues in length to about 50 nucleic acid residues in length, or about 45 nucleic acid residues in length to about 50 nucleic acid residues in length. In some aspects, the linker is about 6 nucleic acid residues in length, about 8 nucleic acid residues in length, about 10 nucleic acid residues in length, about 12 nucleic acid residues in length, about 15 nucleic acid residues in length, about 20 nucleic acid residues in length, about 25 nucleic acid residues in length, about 30 nucleic acid residues in length, about 35 nucleic acid residues in length, about 40 nucleic acid residues in length, about 45 nucleic acid residues in length, or about 50 nucleic acid residues in length. In some aspects, the linker is at least about 6 nucleic acid residues in length, about 8 nucleic acid residues in length, about 10 nucleic acid residues in length, about 12 nucleic acid residues in length, about 15 nucleic acid residues in length, about 20 nucleic acid residues in length, about 25 nucleic acid residues in length, about 30 nucleic acid residues in length, about 35 nucleic acid residues in length, about 40 nucleic acid residues in length, or about 45 nucleic acid residues in length. In some aspects, the linker is at most about 8 nucleic acid residues in length, about 10 nucleic acid residues in length, about 12 nucleic acid residues in length, about 15 nucleic acid residues in length, about 20 nucleic acid residues in length, about 25 nucleic acid residues in length, about 30 nucleic acid residues in length, about 35 nucleic acid residues in length, about 40 nucleic acid residues in length, about 45 nucleic acid residues in length, or about 50 nucleic acid residues in length.

In some aspects, the nucleic acid sequence encoding or comprising the linker is about 6 to about 15 nucleic acid residues in length. In some aspects, the linker is about 6 nucleic acid residues in length to about 7 nucleic acid residues in length, about 6 nucleic acid residues in length to about 8 nucleic acid residues in length, about 6 nucleic acid residues in length to about 9 nucleic acid residues in length, about 6 nucleic acid residues in length to about 10 nucleic acid residues in length, about 6 nucleic acid residues in length to about 11 nucleic acid residues in length, about 6 nucleic acid residues in length to about 12 nucleic acid residues in length, about 6 nucleic acid residues in length to about 13 nucleic acid residues in length, about 6 nucleic acid residues in length to about 14 nucleic acid residues in length, about 6 nucleic acid residues in length to about 15 nucleic acid residues in length, about 7 nucleic acid residues in length to about 8 nucleic acid residues in length, about 7 nucleic acid residues in length to about 9 nucleic acid residues in length, about 7 nucleic acid residues in length to about 10 nucleic acid residues in length, about 7 nucleic acid residues in length to about 11 nucleic acid residues in length, about 7 nucleic acid residues in length to about 12 nucleic acid residues in length, about 7 nucleic acid residues in length to about 13 nucleic acid residues in length, about 7 nucleic acid residues in length to about 14 nucleic acid residues in length, about 7 nucleic acid residues in length to about 15 nucleic acid residues in length, about 8 nucleic acid residues in length to about 9 nucleic acid residues in length, about 8 nucleic acid residues in length to about 10 nucleic acid residues in length, about 8 nucleic acid residues in length to about 11 nucleic acid residues in length, about 8 nucleic acid residues in length to about 12 nucleic acid residues in length, about 8 nucleic acid residues in length to about 13 nucleic acid residues in length, about 8 nucleic acid residues in length to about 14 nucleic acid residues in length, about 8 nucleic acid residues in length to about 15 nucleic acid residues in length, about 9 nucleic acid residues in length to about 10 nucleic acid residues in length, about 9 nucleic acid residues in length to about 11 nucleic acid residues in length, about 9 nucleic acid residues in length to about 12 nucleic acid residues in length, about 9 nucleic acid residues in length to about 13 nucleic acid residues in length, about 9 nucleic acid residues in length to about 14 nucleic acid residues in length, about 9 nucleic acid residues in length to about 15 nucleic acid residues in length, about 10 nucleic acid residues in length to about 11 nucleic acid residues in length, about 10 nucleic acid residues in length to about 12 nucleic acid residues in length, about 10 nucleic acid residues in length to about 13 nucleic acid residues in length, about 10 nucleic acid residues in length to about 14 nucleic acid residues in length, about 10 nucleic acid residues in length to about 15 nucleic acid residues in length, about 11 nucleic acid residues in length to about 12 nucleic acid residues in length, about 11 nucleic acid residues in length to about 13 nucleic acid residues in length, about 11 nucleic acid residues in length to about 14 nucleic acid residues in length, about 11 nucleic acid residues in length to about 15 nucleic acid residues in length, about 12 nucleic acid residues in length to about 13 nucleic acid residues in length, about 12 nucleic acid residues in length to about 14 nucleic acid residues in length, about 12 nucleic acid residues in length to about 15 nucleic acid residues in length, about 13 nucleic acid residues in length to about 14 nucleic acid residues in length, about 13 nucleic acid residues in length to about 15 nucleic acid residues in length, or about 14 nucleic acid residues in length to about 15 nucleic acid residues in length. In some aspects, the linker is about 6 nucleic acid residues in length, about 7 nucleic acid residues in length, about 8 nucleic acid residues in length, about 9 nucleic acid residues in length, about 10 nucleic acid residues in length, about 11 nucleic acid residues in length, about 12 nucleic acid residues in length, about 13 nucleic acid residues in length, about 14 nucleic acid residues in length, or about 15 nucleic acid residues in length. In some aspects, the linker is at least about 6 nucleic acid residues in length, about 7 nucleic acid residues in length, about 8 nucleic acid residues in length, about 9 nucleic acid residues in length, about 10 nucleic acid residues in length, about 11 nucleic acid residues in length, about 12 nucleic acid residues in length, about 13 nucleic acid residues in length, or about 14 nucleic acid residues in length. In some aspects, the linker is at most about 7 nucleic acid residues in length, about 8 nucleic acid residues in length, about 9 nucleic acid residues in length, about 10 nucleic acid residues in length, about 11 nucleic acid residues in length, about 12 nucleic acid residues in length, about 13 nucleic acid residues in length, about 14 nucleic acid residues in length, or about 15 nucleic acid residues in length.

In some embodiments, the recombinant polynucleic acid construct is circular. In some embodiments, the recombinant polynucleic acid construct is linear. In some embodiments, the recombinant polynucleic acid is DNA. In some embodiments, the recombinant polynucleic acid is RNA.

In some embodiments, the recombinant polynucleic acid construct further comprises a promoter. In some embodiments, the promoter is upstream of the at least one nucleic acid sequence encoding or comprising the siRNA. Non-limiting examples of promoters include T3, T7, SP6, P60, Syn5, and KP34, etc. In some embodiments, the recombinant polynucleic acid construct comprises a T3 promoter. In some embodiments, the recombinant polynucleic acid construct comprises a SP6 promoter. In some embodiments, the recombinant polynucleic acid construct comprises a P60 promoter. In some embodiments, the recombinant polynucleic acid construct comprises a Syn5 promoter. In some embodiments, the recombinant polynucleic acid construct comprises a KP34 promoter. In a preferred embodiment, the recombinant polynucleic acid construct comprises a T7 promoter. In some embodiments, the T7 promoter comprises a sequence comprising TAATACGACTCACTATA (SEQ ID NO: 25). In some embodiments, the recombinant polynucleic acid or RNA construct further comprises a Kozak sequence.

In some embodiments, the recombinant polynucleic acid or RNA construct may be codon-optimized. In some embodiments, the recombinant polynucleic acid used in the present invention to transcribe the recombinant RNA construct of the present invention and the recombinant RNA construct of the present invention are codon-optimized. In general, codon optimization refers to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, Pa.) and GeneOptimizer® (ThermoFischer, Mass.). In some embodiments, the recombinant polynucleic acid or RNA construct may not be codon-optimized.

In some aspects, provided herein, is a composition comprising a recombinant polynucleic acid or RNA construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest. In some embodiments, the recombinant polynucleic acid or RNA construct may comprise two or more nucleic acid sequences encoding or comprising a small interfering RNA (siRNA) capable of binding to a target messenger RNA (mRNA) and two or more nucleic acid sequences encoding a gene of interest. In some embodiments, the recombinant nucleic acid or RNA construct may comprise 2, 3, 4, 5, 6, 7, 8, 9, 10, 11, 12, 13, 14, 15, or more nucleic acid sequences encoding an siRNA capable of binding to a target mRNA. In this embodiment, each of the two or more nucleic acid sequences may encode or comprise an siRNA capable of binding to a same target mRNA or a different target mRNA. In one embodiment, each of the two or more nucleic acid sequences may encode or comprise an siRNA capable of binding to a same target mRNA. In another embodiment, each of the two or more nucleic acid sequences may encode or comprise an siRNA capable of binding to a different target mRNA. In some embodiments, the recombinant nucleic acid or RNA construct may comprise two or more nucleic acid sequences encoding a gene of interest. In some embodiments, the recombinant nucleic acid or RNA construct may comprise 2, 3, 4, 5, 6, 7, 8, 9, 10, 11, 12, 13, 14, 15, or more nucleic acid sequences encoding a gene of interest. In this embodiment, each of the two or more nucleic acid sequences may encode a same gene of interest or a different gene of interest, wherein the mRNA encoded by the same or the different gene of interest is different from the siRNA target mRNA. In one embodiment, each of the two or more nucleic acid sequences may encode a same gene of interest, wherein the mRNA encoded by the same gene of interest is different from the siRNA target mRNA. In another embodiment, each of the two or more nucleic acid sequences may encode a different gene of interest, wherein the mRNA encoded by the different gene of interest is different from the siRNA target mRNA.

In some embodiments, the recombinant polynucleic acid or RNA construct may comprise two or more nucleic acid sequences encoding or comprising an siRNA capable of binding to a target mRNA and at least one nucleic acid sequence encoding a gene of interest, wherein each of the two or more nucleic acid sequences encoding or comprising an siRNA encodes or comprises an siRNA capable of binding to a same target mRNA, wherein the at least one nucleic acid sequence encoding a gene of interest encodes a same gene of interest, and wherein the mRNA encoded by the same gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and at least one nucleic acid sequence encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a same target mRNA, wherein the at least one nucleic acid sequence encoding a gene of interest encodes a same gene of interest, and wherein the mRNA encoded by the same gene of interest is different from the siRNA target mRNA.

In some embodiments, the recombinant polynucleic acid or RNA construct may comprise two or more nucleic acid sequences encoding or comprising an siRNA capable of binding to a target mRNA and at least one nucleic acid sequence encoding a gene of interest, wherein each of the two or more nucleic acid sequences encoding or comprising an siRNA encodes or comprises an siRNA capable of binding to a different target mRNA, wherein the at least one nucleic acid sequence encoding a gene of interest encodes a same gene of interest, and wherein the mRNA encoded by the same gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and at least one nucleic acid sequence encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a different target mRNA, wherein the at least one nucleic acid sequence encoding a gene of interest encodes a same gene of interest, and wherein the mRNA encoded by the same gene of interest is different from the siRNA target mRNA.

In some embodiments, the recombinant polynucleic acid or RNA construct may comprise at least one nucleic acid sequence encoding or comprising an siRNA capable of binding to a target mRNA and two or more nucleic acid sequences encoding a gene of interest, wherein the at least one nucleic acid sequence encoding or comprising an siRNA encodes or comprises an siRNA capable of binding to a same target mRNA, wherein each of the two or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise at least one nucleic acid sequence encoding or comprising an siRNA capable of binding to a target mRNA and 3, 4, 5, 6, 7, 8, 9, 10, 11, 12, 13, 14, 15, or more nucleic acid sequences encoding a gene of interest, wherein the at least one nucleic acid encoding or comprising an siRNA encodes or comprises an siRNA capable of binding to a same target mRNA, wherein the each of the 3, 4, 5, 6, 7, 8, 9, 10, 11, 12, 13, 14, 15, or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise at least one nucleic acid sequence encoding or comprising an siRNA capable of binding to a target mRNA and two or more nucleic acid sequences encoding a gene of interest, wherein the at least one nucleic acid sequence encoding or comprising an siRNA encodes or comprises an siRNA capable of binding to a same target mRNA, wherein each of the two or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise at least one nucleic acid sequence encoding or comprising an siRNA capable of binding to a target mRNA and 3, 4, 5, 6, 7, 8, 9, 10, 11, 12, 13, 14, 15, or more nucleic acid sequences encoding a gene of interest, wherein the at least one nucleic acid encoding or comprising an siRNA encodes or comprises an siRNA capable of binding to a same target mRNA, wherein the each of the 3, 4, 5, 6, 7, 8, 9, 10, 11, 12, 13, 14, 15, or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise two or more nucleic acid sequences encoding or comprising an siRNA capable of binding to a target mRNA and two or more nucleic acid sequences encoding a gene of interest, wherein each of the two or more nucleic acid sequences encoding or comprising an siRNA encodes or comprises an siRNA capable of binding to a same target mRNA, wherein each of the two or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise two or more nucleic acid sequences encoding or comprising an siRNA capable of binding to a target mRNA and two or more nucleic acid sequences encoding a gene of interest, wherein each of the two or more nucleic acid sequences encoding or comprising an siRNA encodes or comprises an siRNA capable of binding to a different target mRNA, wherein each of the two or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise two or more nucleic acid sequences encoding or comprising an siRNA capable of binding to a target mRNA and two or more nucleic acid sequences encoding a gene of interest, wherein each of the two or more nucleic acid sequences encoding or comprising an siRNA encodes or comprises an siRNA capable of binding to a same target mRNA, wherein each of the two or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise two or more nucleic acid sequences encoding or comprising an siRNA capable of binding to a target mRNA and two or more nucleic acid sequences encoding a gene of interest, wherein each of the two or more nucleic acid sequences encoding or comprising an siRNA encodes or comprises an siRNA capable of binding to a different target mRNA, wherein each of the two or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and two or more nucleic acid sequences encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a same target mRNA, wherein each of the two or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and two or more nucleic acid sequences encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a different target mRNA, wherein each of the two or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and two or more nucleic acid sequences encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a same target mRNA, wherein each of the two or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise 3, 4, 5, 6, 7, 8, 9, 10, 11, 12, 13, 14, 15, or more nucleic acid sequences encoding or comprising an siRNA capable of binding to a target mRNA and two or more nucleic acid sequences encoding a gene of interest, wherein each of the 3, 4, 5, 6, 7, 8, 9, 10, 11, 12, 13, 14, 15, or more nucleic acid sequences encoding or comprising an siRNA encodes or comprises an siRNA capable of binding to a different target mRNA, wherein each of the two or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise two or more nucleic acid sequences encoding or comprising an siRNA capable of binding to a target mRNA and 3, 4, 5, 6, 7, 8, 9, 10, 11, 12, 13, 14, 15, or more nucleic acid sequences encoding a gene of interest, wherein each of the two or more nucleic acid sequences encoding or comprising an siRNA encodes or comprises an siRNA capable of binding to a same target mRNA, wherein each of the 3, 4, 5, 6, 7, 8, 9, 10, 11, 12, 13, 14, 15, or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise two or more nucleic acid sequences encoding or comprising an siRNA capable of binding to a target mRNA and 3, 4, 5, 6, 7, 8, 9, 10, 11, 12, 13, 14, 15, or more nucleic acid sequences encoding a gene of interest, wherein each of the two or more nucleic acid sequences encoding or comprising an siRNA encodes or comprises an siRNA capable of binding to a different target mRNA, wherein each of the 3, 4, 5, 6, 7, 8, 9, 10, 11, 12, 13, 14, 15, or more nucleic acid sequences encoding a gene of interest encodes a same gene of interest, and wherein the mRNA encoded by the same gene of interest is different from the siRNA target mRNA.

In some embodiments, the recombinant polynucleic acid or RNA construct may comprise two or more nucleic acid sequences encoding or comprising an siRNA capable of binding to a target mRNA and 3, 4, 5, 6, 7, 8, 9, 10, 11, 12, 13, 14, 15, or more nucleic acid sequences encoding a gene of interest, wherein each of the two or more nucleic acid sequences encoding or comprising an siRNA encodes or comprises an siRNA capable of binding to a same target mRNA, wherein each of the 3, 4, 5, 6, 7, 8, 9, 10, 11, 12, 13, 14, 15, or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise two or more nucleic acid sequences encoding or comprising an siRNA capable of binding to a target mRNA and 3, 4, 5, 6, 7, 8, 9, 10, 11, 12, 13, 14, 15, or more nucleic acid sequences encoding a gene of interest, wherein each of the two or more nucleic acid sequences encoding or comprising an siRNA encodes or comprises an siRNA capable of binding to a different target mRNA, wherein each of the 3, 4, 5, 6, 7, 8, 9, 10, 11, 12, 13, 14, 15, or more nucleic acid sequences encoding a gene of interest encodes a different gene of interest, and wherein the mRNA encoded by the different gene of interest is different from the siRNA target mRNA.

In some embodiments, the recombinant polynucleic acid or RNA construct may comprise three or more nucleic acid sequences encoding or comprising an siRNA capable of binding to a target mRNA and at least one nucleic acid sequence encoding a gene of interest, wherein one or more of the three or more nucleic acid sequences encoding or comprising an siRNA encodes or comprises an siRNA capable of binding to a first target mRNA and one or more of the three or more nucleic acid sequences encoding or comprising an siRNA encodes or comprises an siRNA capable of binding to a second target mRNA, wherein the first and the second target mRNA are different, wherein the at least one nucleic acid sequence encoding a gene of interest encodes a same gene of interest, and wherein the mRNA encoded by the same gene of interest is different from the first and the second target mRNA that the siRNA is capable of binding to. For example, the recombinant polynucleic acid or RNA construct of the present invention may comprise five nucleic acid sequences encoding or comprising an siRNA capable of binding to a target mRNA and at least one nucleic acid sequence encoding a gene of interest, wherein three of the five nucleic acid sequences encoding or comprising an siRNA encodes or comprises an siRNA capable of binding to a first target mRNA and the other two of the five nucleic acid sequences encoding or comprising an siRNA encodes or comprises an siRNA capable of binding to a second target mRNA, wherein the first and the second target mRNA are different, wherein the at least one nucleic acid sequence encoding a gene of interest encodes a same gene of interest, and wherein the mRNA encoded by the same gene of interest is different from the first and the second target mRNA that the siRNA is capable of binding to.

In some embodiments, the recombinant polynucleic acid or RNA construct may comprise three or more nucleic acid sequences encoding or comprising an siRNA capable of binding to a target mRNA and at least one nucleic acid sequence encoding a gene of interest, wherein one or more of the three or more nucleic acid sequences encoding or comprising an siRNA encodes or comprises an siRNA capable of binding to a first target mRNA, another one or more of the three or more nucleic acid sequences encoding or comprising an siRNA encodes or comprises an siRNA capable of binding to a second target mRNA, and the other one or more of the three or more nucleic acid sequences encoding or comprising an siRNA encodes or comprises an siRNA capable of binding to a third target mRNA, wherein the first, the second, and the third target mRNAs are different, wherein the at least one nucleic acid sequence encoding a gene of interest encodes a same gene of interest, and wherein the mRNA encoded by the same gene of interest is different from the first, the second, and the third target mRNA that the siRNA is capable of binding to. For example, the recombinant polynucleic acid or RNA construct of the present invention may comprise five nucleic acid sequences encoding or comprising an siRNA capable of binding to a target mRNA and at least one nucleic acid sequence encoding a gene of interest, wherein two of the five nucleic acid sequences encoding or comprising an siRNA encodes or comprises an siRNA capable of binding to a first target mRNA, one of the five nucleic acid sequences encoding or comprising an siRNA encodes or comprises an siRNA capable of binding to a second target mRNA, and one of the five nucleic acid sequences encoding or comprising an siRNA encodes or comprises an siRNA capable of binding to a third target mRNA, wherein the first target mRNA, the second target mRNA, and the third target mRNA are different, wherein the at least one nucleic acid sequence encoding a gene of interest encodes a same gene of interest, and wherein the mRNA encoded by the same gene of interest is different from the first, the second, and the third target mRNA that the siRNA is capable of binding to.

In some embodiments, the recombinant polynucleic acid or RNA construct may comprise at least one nucleic acid sequence encoding or comprising an siRNA capable of binding to a target mRNA and three or more nucleic acid sequences encoding a gene of interest, wherein at least one nucleic acid sequence encoding or comprising an siRNA encodes or comprises an siRNA capable of binding to a same target mRNA, wherein one or more of the three or more nucleic acid sequences encoding a gene of interest encodes a first gene of interest and one or more of the three or more nucleic acid sequences encoding a gene of interest encodes a second gene of interest, wherein the first gene of interest and the second gene of interest are different, and wherein the mRNAs encoded by the first gene of interest and the second gene of interest are different from the siRNA target mRNA. For example, the recombinant polynucleic acid or RNA construct of the present invention may comprise at least one nucleic acid sequence encoding or comprising an siRNA capable of binding to a target mRNA and five nucleic acid sequences encoding a gene of interest, wherein at least one nucleic acid sequence encoding or comprising an siRNA encodes or comprises an siRNA capable of binding to a same target mRNA, wherein three of the five nucleic acid sequences encoding a gene of interest encodes a first gene of interest and two of the five nucleic acid sequences encoding a gene of interest encodes a second gene of interest, wherein the first gene of interest and the second gene of interest are different, and wherein the mRNAs encoded by the first gene of interest and the second gene of interest are different from the siRNA target mRNA.

In some embodiments, the recombinant polynucleic acid or RNA construct may comprise at least one nucleic acid sequence encoding or comprising an siRNA capable of binding to a target mRNA and three or more nucleic acid sequences encoding a gene of interest, wherein at least one nucleic acid sequence encoding or comprising an siRNA encodes or comprises an siRNA capable of binding to a same target mRNA, wherein one or more of the three or more nucleic acid sequences encoding a gene of interest encodes a first gene of interest, one or more of the three or more nucleic acid sequences encoding a gene of interest encodes a second gene of interest, and one or more of the three or more nucleic acid sequences encoding a gene of interest encodes a third gene of interest, wherein the first gene of interest, the second gene of interest, and the third gene of interest are different, and wherein the mRNAs encoded by the first gene of interest, the second gene of interest, and the third gene of interest are different from the siRNA target mRNA. For example, the recombinant polynucleic acid or RNA construct of the present invention may comprise at least one nucleic acid sequence encoding or comprising an siRNA capable of binding to a target mRNA and five nucleic acid sequences encoding a gene of interest, wherein at least one nucleic acid sequence encoding or comprising an siRNA encodes or comprises an siRNA capable of binding to a same target mRNA, wherein three of the five nucleic acid sequences encoding a gene of interest encodes a first gene of interest, one of the five nucleic acid sequences encoding a gene of interest encodes a second gene of interest, and one of the five nucleic acid sequences encoding a gene of interest encodes a third gene of interest, wherein the first gene of interest, the second gene of interest, and the third gene of interest are different, and wherein the mRNAs encoded by the first gene of interest, the second gene of interest, and the third gene of interest are different from the siRNA target mRNA.

In some embodiments, the recombinant polynucleic acid or RNA construct may comprise three or more nucleic acid sequences encoding or comprising an siRNA capable of binding to a target mRNA and three or more nucleic acid sequences encoding a gene of interest, wherein one or more of the three or more nucleic acid sequences encoding or comprising an siRNA encodes or comprises an siRNA capable of binding to a first target mRNA and one or more of the three or more nucleic acid sequences encoding or comprising an siRNA encodes or comprises an siRNA capable of binding to a second target mRNA, wherein the first and the second target mRNA are different, wherein the one or more of the three or more nucleic acid sequences encoding a gene of interest encodes a first gene of interest and one or more of the three or more nucleic acid sequences encoding a gene of interest encodes a second gene of interest, wherein the first gene of interest and the second gene of interest are different, and wherein the mRNAs encoded by the first gene of interest and the second gene of interest are different from the first and the second target mRNA that the siRNA is capable of binding to. For example, the recombinant polynucleic acid or RNA construct of the present invention may comprise five nucleic acid sequences encoding or comprising an siRNA capable of binding to a target mRNA and five nucleic acid sequences encoding a gene of interest, wherein three of the five nucleic acid sequences encoding or comprising an siRNA encodes or comprises an siRNA capable of binding to a first target mRNA and the other two of the five nucleic acid sequences encoding or comprising an siRNA encodes or comprises an siRNA capable of binding to a second target mRNA, wherein the first and the second target mRNA are different, wherein three of the five nucleic acid sequences encoding a gene of interest encodes a first gene of interest and two of the five nucleic acid sequences encoding a gene of interest encodes a second gene of interest, wherein the first gene of interest and the second gene of interest are different, and wherein the mRNAs encoded by the first gene of interest and the second gene of interest are different from the first and the second target mRNA that the siRNA is capable of binding to.

In some embodiments, the recombinant polynucleic acid or RNA construct may comprise three or more nucleic acid sequences encoding or comprising an siRNA capable of binding to a target mRNA and three or more nucleic acid sequences encoding a gene of interest, wherein one or more of the three or more nucleic acid sequences encoding or comprising an siRNA encodes or comprises an siRNA capable of binding to a first target mRNA, another one or more of the three or more nucleic acid sequences encoding or comprising an siRNA encodes or comprises an siRNA capable of binding to a second target mRNA, and the other one or more of the three or more nucleic acid sequences encoding or comprising an siRNA encodes or comprises an siRNA capable of binding to a third target mRNA, wherein the first, the second, and the third target mRNAs are different, wherein one or more of the three or more nucleic acid sequences encoding a gene of interest encodes a first gene of interest, one or more of the three or more nucleic acid sequences encoding a gene of interest encodes a second gene of interest, and one or more of the three or more nucleic acid sequences encoding a gene of interest encodes a third gene of interest, wherein the first gene of interest, the second gene of interest, and the third gene of interest are different, and wherein the mRNAs encoded by the first gene of interest, the second gene of interest, and the third gene of interest are different from the first, the second, and the third target mRNA that the siRNA is capable of binding to. For example, the recombinant polynucleic acid or RNA construct of the present invention may comprise five nucleic acid sequences encoding or comprising an siRNA capable of binding to a target mRNA and five nucleic acid sequences encoding a gene of interest, wherein two of the five nucleic acid sequences encoding or comprising an siRNA encodes or comprises an siRNA capable of binding to a first target mRNA, one of the five nucleic acid sequences encoding or comprising an siRNA encodes or comprises an siRNA capable of binding to a second target mRNA, and one of the five nucleic acid sequences encoding or comprising an siRNA encodes or comprises an siRNA capable of binding to a third target mRNA, wherein the first target mRNA, the second target mRNA, and the third target mRNA are different, and wherein three of the five nucleic acid sequences encoding a gene of interest encodes a first gene of interest, one of the five nucleic acid sequences encoding a gene of interest encodes a second gene of interest, and one of the five nucleic acid sequences encoding a gene of interest encodes a third gene of interest, wherein the first gene of interest, the second gene of interest, and the third gene of interest are different, and wherein the mRNAs encoded by the first gene of interest, the second gene of interest, and the third gene of interest are different from the first, the second, and the third target mRNA that the siRNA is capable of binding to.

In some embodiments wherein multiple genes of interest are encoded by a polynucleotide construct, all genes of interest encode the same protein. In some embodiments, all genes of interest encode different proteins. In some embodiments, more than one gene of interest encodes the same protein and at least one gene of interest encodes a different protein. In some embodiments, wherein multiple siRNAs are encoded or comprised by a polynucleotide construct, all siRNAs encoded or comprised by a polynucleotide construct are capable of binding to the same RNA. In some embodiments, all siRNAs are capable of binding to different target RNAs. In some embodiments, more than one siRNA is capable of binding to the same target RNA and at least one siRNA is capable of binding to a different target RNA. In some embodiments, the target RNA is an mRNA. In some embodiments, the target RNA is a noncoding RNA. In some embodiments, wherein multiple siRNAs encoded or comprised by the polynucleotide construct are capable of binding to the same target RNA, all or some of the siRNAs are capable of binding to the same or different target RNA binding sites.

### Recombinant RNA Construct

In one embodiment of the present invention, the recombinant polynucleic acid construct is a recombinant RNA construct. In some embodiments, the recombinant RNA construct is naked RNA. In a preferred embodiment, the recombinant RNA construct comprises a 5′ cap (e.g., an anti-reverse CAP analog, Clean Cap, Cap 0, Cap 1, Cap 2, or Locked Nucleic Acid cap (LNA-cap), etc.), an internal ribosome entry site (IRES), and/or a poly(A) tail at the 3′ end in a particular in order to improve translation. In some embodiments, the recombinant RNA construct has further regions promoting translation known to any skilled artisan. In some embodiments, the 5′ cap comprises an anti-reverse CAP analog, Clean Cap, Cap 0, Cap 1, Cap 2, or Locked Nucleic Acid cap (LNA-cap). In some embodiments, 5′ cap comprises m2^{7,3′-O}G(5′)ppp(5′)G, m7G, m7G(5′)G, m7GpppG, or m7GpppGm.

In some embodiments, the recombinant polynucleic acid construct further comprises a nucleic acid sequence encoding a poly(A) tail. In some embodiments, the recombinant RNA construct comprises a poly(A) tail.

In some embodiments, the poly(A) tail comprises 1, 3, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or 220 base pairs of poly(A) (SEQ ID NO: 192). In some embodiments, the poly(A) tail comprises 1 to 220 base pairs of poly(A) (SEQ ID NO: 191). In some embodiments, the poly(A) tail comprises 1 to 20, 1 to 40, 1 to 60, 1 to 80, 1 to 100, 1 to 120, 1 to 140, 1 to 160, 1 to 180, 1 to 200, 1 to 220, 20 to 40, 20 to 60, 20 to 80, 20 to 100, 20 to 120, 20 to 140, 20 to 160, 20 to 180, 20 to 200, 20 to 220, 40 to 60, 40 to 80, 40 to 100, 40 to 120, 40 to 140, 40 to 160, 40 to 180, 40 to 200, 40 to 220, 60 to 80, 60 to 100, 60 to 120, 60 to 140, 60 to 160, 60 to 180, 60 to 200, 60 to 220, 80 to 100, 80 to 120, 80 to 140, 80 to 160, 80 to 180, 80 to 200, 80 to 220, 100 to 120, 100 to 140, 100 to 160, 100 to 180, 100 to 200, 100 to 220, 120 to 140, 120 to 160, 120 to 180, 120 to 200, 120 to 220, 140 to 160, 140 to 180, 140 to 200, 140 to 220, 160 to 180, 160 to 200, 160 to 220, 180 to 200, 180 to 220, or 200 to 220 base pairs of poly(A) (SEQ ID NO: 194). In some embodiments, the poly(A) tail comprises 1, 20, 40, 60, 80, 100, 120, 140, 160, 180, 200, or 220 base pairs of poly(A) (SEQ ID NO: 195). In some embodiments, the poly(A) tail comprises at least 1, 20, 40, 60, 80, 100, 120, 140, 160, 180, or 200 base pairs of poly(A) (SEQ ID NO: 199). In some embodiments, the poly(A) tail comprises at most 20, 40, 60, 80, 100, 120, 140, 160, 180, 200, or 220 base pairs of poly(A) (SEQ ID NO: 196). In a preferred embodiment, the poly(A) tail comprises 120 base pairs of poly(A) (SEQ ID NO: 193).

In one embodiment of the present invention, the recombinant RNA construct may contain a combination of modified and unmodified nucleotides. In a preferred embodiment, in such a modified recombinant RNA construct, 1 to 100%, preferably 10 to 100%, more preferably 50 to 100%, even more preferably 90 to 100%, most preferably 100% of the uridine nucleotides may be modified. In some embodiments, recombinant RNA constructs transcribed from any DNA constructs described herein may comprise modified uridines. In a preferred embodiment, 100% of uridine nucleotides in recombinant RNA constructs transcribed from any DNA constructs described herein are modified. In some embodiments, the adenosine-, guanosine-, and cytidine-containing nucleotides are unmodified or partially modified, and they are preferably present in unmodified form. Preferably the content of the modified uridine nucleotides in the recombinant RNA construct may lie in a range from 5 to 25%. Non-limiting examples of the modified uridine nucleotides may comprise pseudouridines, N¹-Methylpseudouridines, or N1-methylpseudo-UTP and any modified uridine nucleotides known in the art may be utilized. In some embodiments, the recombinant RNA construct may contain a combination of modified and unmodified nucleotides, wherein in such a modified recombinant RNA construct, 1 to 100%, preferably 10 to 100%, more preferably 50 to 100%, even more preferably 90 to 100%, most preferably 100% of the uridine nucleotides may comprise pseudouridines, N¹-Methylpseudouridines, N1-methylpseudo-UTP, or any other modified uridine nucleotide known in the art. In some embodiments, the recombinant RNA construct may contain a combination of modified and unmodified nucleotides, wherein in such a modified recombinant RNA construct, 1 to 100%, preferably 10 to 100%, more preferably 50 to 100%, even more preferably 90 to 100%, most preferably 100% of the uridine nucleotides may comprise N¹-Methylpseudouridines. In some embodiments, recombinant RNA constructs transcribed from any DNA constructs described herein may comprise N¹-Methylpseudouridines. In a preferred embodiment, 100% of uridine nucleotides in recombinant RNA constructs transcribed from any DNA constructs described herein are modified to N¹-Methylpseudouridines.

In some embodiments, the recombinant RNA construct may be codon-optimized. In general, codon optimization refers to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, Pa.) and GeneOptimizer® (ThermoFischer, Mass.) which is preferred. In some embodiments, the recombinant RNA construct may not be codon-optimized.

In a preferred embodiment the present invention comprises a composition comprising a recombinant RNA construct comprising: (i) a small interfering RNA (siRNA) capable of binding to Interleukin 8 (IL-8) messenger RNA (mRNA); and (ii) an mRNA encoding Insulin-like Growth Factor 1 (IGF-1).

In another preferred embodiment the present invention comprises a composition comprising a recombinant RNA construct comprising: (i) a small interfering RNA (siRNA) capable of binding to Interleukin 1 beta (IL-1 beta) messenger RNA (mRNA); and (ii) an mRNA encoding Insulin-like Growth Factor 1 (IGF-1).

In another preferred embodiment the present invention comprises a composition comprising a recombinant RNA construct comprising: (i) a small interfering RNA (siRNA) capable of binding to Interleukin 17 (IL-17) messenger RNA (mRNA); and (ii) an mRNA encoding Interleukin 4 (IL-4).

In another preferred embodiment the present invention comprises a composition comprising a recombinant RNA construct comprising: (i) a small interfering RNA (siRNA) capable of binding to Tumor Necrosis Factor alpha (TNF-alpha or TNF-α) messenger RNA (mRNA); and (ii) an mRNA encoding Interleukin 4 (IL-4).

In another preferred embodiment the present invention comprises a composition comprising a recombinant RNA construct comprising: (i) a small interfering RNA (siRNA) capable of binding to Tumor Necrosis Factor alpha (TNF-alpha) messenger RNA (mRNA) and a small interfering RNA (siRNA) capable of binding to Interleukin 17 (IL-17) messenger RNA (mRNA); and (ii) an mRNA encoding Interleukin 4 (IL-4).

In another preferred embodiment the present invention is a composition comprising a polynucleic acid construct comprising a nucleic acid sequence selected from the group consisting of SEQ ID NOs: 1-8.

In another preferred embodiment the present invention is a composition comprising a polynucleic acid construct, e.g., a recombinant RNA construct, comprising a nucleic acid sequence selected from the group consisting of SEQ ID NOs: 29-47.

In another preferred embodiment the present invention is a composition comprising a polynucleic acid construct comprising a nucleic acid sequence selected from the group consisting of SEQ ID NOs: 1-8 and SEQ ID NOs: 29-47.

In another preferred embodiment the present invention comprises a composition comprising a recombinant RNA construct comprising: (i) a small interfering RNA (siRNA) capable of binding to Activin receptor-like kinase-2 (ALK2) messenger RNA (mRNA); and (ii) an mRNA encoding Insulin-like Growth Factor 1 (IGF-1).

In another preferred embodiment the present invention comprises a composition comprising a recombinant RNA construct comprising: (i) a small interfering RNA (siRNA) capable of binding to Superoxide dismutase-1 (SOD1) messenger RNA (mRNA); and (ii) an mRNA encoding Insulin-like Growth Factor 1 (IGF-1).

In another preferred embodiment the present invention comprises a composition comprising a recombinant RNA construct comprising: (i) a small interfering RNA (siRNA) capable of binding to Superoxide dismutase-1 (SOD1) messenger RNA (mRNA); and (ii) an mRNA encoding Erythropoietin (EPO).

In another preferred embodiment the present invention is a composition comprising a polynucleic acid construct comprising a nucleic acid sequence selected from the group consisting of SEQ ID NOs: 152-158.

In some embodiments, the recombinant polynucleic acid construct described herein comprises a sequence with at least 50%, 55%, 60%, 65%, 70%, 75%, 80%, 85%, 90%, 95%, or at least 99% sequence identity to any one of SEQ ID NOs: 177-189. In some embodiments, the recombinant polynucleic acid construct described herein comprises a sequence with at least 50%, 55%, 60%, 65%, 70%, 75%, 80%, 85%, 90%, 95%, or at least 99% sequence identity to SEQ ID NO: 190.

In another preferred embodiment the present invention is a composition comprising a polynucleic acid construct comprising a nucleic acid sequence selected from the group consisting of SEQ ID NOs: 177-189.

In another preferred embodiment the present invention is a composition comprising a polynucleic acid construct comprising a nucleic acid sequence of SEQ ID NO: 190.

In some aspects, provided herein, is a method of producing an RNA construct comprising an siRNA capable of binding to a target mRNA and mRNA encoding a gene of interest. In some embodiments, the RNA construct is produced by in vitro transcription. In this embodiment, (i) a polynucleic acid construct comprising a promoter, at least one nucleic acid sequence encoding an siRNA capable of binding to a target mRNA, at least one nucleic acid sequence encoding a gene of interest, and a nucleic acid sequence encoding poly(A) tail; (ii) an RNA polymerase; and (iii) a mixture of nucleotide triphosphates (NTPs) is provided for the in vitro (“cell free”) transcription. Details of producing RNA using in vitro transcription as well as isolating and purifying transcribed RNAs is well known in the art and can be found, for example, in Beckert & Masquida ((2011) Synthesis of RNA by In vitro Transcription. RNA. Methods in Molecular Biology (Methods and Protocols), vol 703. Humana Press). A non-limiting list of in vitro transcript kits includes MEGAscript™ T3 Transcription Kit, MEGAscript T7 kit, MEGAscript™ SP6 Transcription Kit, MAXIscript™ T3 Transcription Kit, MAXIscript™ T7 Transcription Kit, MAXIscript™ SP6 Transcription Kit, MAXIscript™ T7/T3 Transcription Kit, MAXIscript™ SP6/T7 Transcription Kit, mMESSAGE mMACHINE™ T3 Transcription Kit, mMESSAGE mMACHINE™ T7 Transcription Kit, mMESSAGE mMACHINE™ SP6 Transcription Kit, MEGAshortscript™ T7 Transcription Kit, HiScribe™ T7 High Yield RNA Synthesis Kit, HiScribe™ T7 In Vitro Transcription Kit, AmpliScribe™ T7-Flash™ Transcription Kit, AmpliScribe™ T7 High Yield Transcription Kit, AmpliScribe™ T7-Flash™ Biotin-RNA Transcription Kit, T7 Transcription Kit, HighYield T7 RNA Synthesis Kit, DuraScribe® T7 Transcription Kit, etc.

In some embodiments, the polynucleic acid construct may be linear. The in vitro transcription reaction can further comprise a transcription buffer system, nucleotide triphosphates (NTPs), and an RNase inhibitor. In some embodiments, the transcription buffer system may comprise dithiothreitol (DTT) and magnesium ions. The NTPs can be naturally occurring or non-naturally occurring (modified) NTPs. Non-limiting examples of non-naturally occurring (modified) NTPs include N¹-methylpseudouridine, Pseudouridine, N¹-Ethylpseudouridine, N¹-Methoxymethylpseudouridine, N¹—Propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-Bromouridine, 5-Iodouridine, 5,6-dihydrouridine, 6-Azauridine, Thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxycytidine, 5-hydroxycytidine, 5-Iodocytidine, 5-Bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-Aminoadenosine, N⁶-isopentenyladenosine, N⁶,N⁶-dimethyladenosine, 7-methyladenine, 2-methylthio-adenine, and 2-methoxy-adenine. Non-limiting examples of DNA-dependent RNA polymerase include T3, T7, SP6, P60, Syn5, and KP34 RNA polymerases. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase. In some embodiments, the RNA polymerase is T3 RNA polymerase. In some embodiments, the RNA polymerase is SP6 RNA polymerase. In some embodiments, the RNA polymerase is P60 RNA polymerase. In some embodiments, the RNA polymerase is Syn5 RNA polymerase. In some embodiments, the RNA polymerase is KP34 RNA polymerase. In a preferred embodiment, the RNA polymerase is T7 RNA polymerase.

In further embodiments, transcribed RNAs may be isolated and purified from the in vitro transcription reaction mixture. In this embodiments, transcribed RNAs may be isolated and purified using column purification. Details of isolating and purifying transcribed RNAs from in vitro transcription reaction mixture is well known in the art and any commercially available kits may be used. A non-limiting list of RNA purification kits includes MEGAclear kit, Monarch® RNA Cleanup Kit, EasyPure® RNA Purification Kit, NucleoSpin® RNA Clean-up, etc.

### Recombinant Polynucleic Acid Construct for Treating a Viral Disease or Condition

The recombinant polynucleic acid construct of the present invention can be directed toward treatment of diseases and conditions related to virus infection. In these embodiments, the recombinant polynucleic acid construct can simultaneously downregulate the expression of one or more proteins and upregulate the expression of one or more proteins by providing a nucleic acid sequence encoding or comprising a single or multiple small interfering RNA (siRNA) species capable of binding to a specific target(s), and a nucleic acid sequence encoding single or multiple proteins for overexpression. In some embodiments, the recombinant polynucleic acid is DNA. In some embodiments, the recombinant polynucleic acid is RNA.

In some aspects, provided herein, is a composition comprising a recombinant polynucleic acid or RNA construct comprising: (i) at least one nucleic acid sequence encoding or comprising a small interfering RNA (siRNA) capable of specifically binding to a target RNA (e.g., an mRNA or a noncoding RNA); and (ii) at least one nucleic acid sequence encoding a gene of interest; wherein the target RNA is different from an mRNA encoded by the gene of interest.

In some embodiments, (i) and (ii) are oriented in a 5′ to 3′ direction (the elements of (i) are upstream of the elements of (ii)). In some embodiments, (i) and (ii) are not oriented in a 5′ to 3′ direction (e.g., the element(s) of (ii) are upstream of the elements of (i)). In some embodiments, the at least one nucleic acid sequence encoding or comprising the small interfering RNA (siRNA) capable of specifically binding to the target RNA (e.g., an mRNA or a noncoding RNA) is upstream of the at least one nucleic acid sequence encoding the gene of interest. In some embodiments, the at least one nucleic acid sequence encoding or comprising the small interfering RNA (siRNA) capable of specifically binding to the target RNA (e.g., an mRNA or a noncoding RNA) is downstream of the at least one nucleic acid sequence encoding the gene of interest. In some embodiments, the recombinant polynucleic acid construct further comprises a nucleic acid sequence encoding or comprising a linker. In some embodiments, the nucleic acid sequence encoding or comprising the linker connects (i) and (ii). In some embodiments, the nucleic acid sequence encoding or comprising the linker connects the at least one nucleic acid sequence encoding or comprising the small interfering RNA (siRNA) capable of specifically binding to the target RNA (e.g., an mRNA or a noncoding RNA) and the at least one nucleic acid sequence encoding the gene of interest. In some embodiments, the linker comprises a tRNA linker. In some embodiments, the recombinant polynucleic acid construct is circular. In some embodiments, the recombinant polynucleic acid construct is linear. In some embodiments, the recombinant polynucleic acid construct is DNA. In some embodiments, the recombinant polynucleic acid construct is RNA. In some embodiments, the recombinant polynucleic acid construct comprises a nucleic acid sequence as set forth in one of SEQ ID NOs: 1-8 or 29-47. In some embodiments, the recombinant polynucleic acid construct comprises a nucleic acid sequence as set forth in one of SEQ ID NOs: 152-158. In some embodiments, the recombinant polynucleic acid construct comprises a nucleic acid sequence as set forth in one of SEQ ID NOs: 177-190.

In some embodiments, the recombinant polynucleic acid construct further comprises a nucleic acid sequence encoding or comprising a poly(A) tail. In some embodiments, the poly(A) tail comprises 1-220 A residues (SEQ ID NO: 191). In some embodiments, the recombinant polynucleic acid construct further comprises a 5′ cap. In some embodiments, the 5′ cap comprises an anti-reverse CAP analog, Clean Cap, Cap 0, Cap 1, Cap 2, or Locked Nucleic Acid cap (LNA-cap). In some embodiments, the 5′ cap comprises m2^{7,3′-O}G(5′)ppp(5′)G, m7G, m7G(5′)G, m7GpppG, or m7GpppGm. In some embodiments, the recombinant polynucleic acid construct further comprises a promoter. In some embodiments, the promoter is selected from the group consisting of T3, T7, SP6, P60, Syn5, and KP34. In some embodiments, the promoter is a T7 promoter. In some embodiments, the T7 promoter is upstream of the at least one nucleic acid sequence encoding or comprising the siRNA. In some embodiments, the T7 promoter is upstream of the at least one nucleic acid sequence encoding or comprising the gene of interest. In some embodiments, the T7 promoter comprises a sequence TAATACGACTCACTATA (SEQ ID NO: 25). In some embodiments, the recombinant polynucleic acid construct further comprises a Kozak sequence. In some embodiments, the Kozak sequence is GCCACC (SEQ ID NO: 26).

In some embodiments, the recombinant polynucleic acid construct encodes or comprises 1-10 siRNA species. In some embodiments, the siRNA species are the same. In some embodiments, the siRNA species are different. In some embodiments, some siRNA species are the same and some are different. In some embodiments, the siRNA comprises a sense siRNA strand. In some embodiments, the siRNA comprises an anti-sense siRNA strand. In some embodiments, the siRNA comprises a sense and an anti-sense siRNA strand. In some embodiments, the siRNA does not affect the expression of the gene of interest. In some embodiments, the siRNA does not inhibit the expression of the gene of interest. In some embodiments, the recombinant polynucleic acid construct comprises two or more nucleic acid sequences encoding or comprising an siRNA capable of binding to a target mRNA. In some embodiments, the recombinant polynucleic acid construct further comprises a nucleic acid sequence encoding or comprising a linker. In some embodiments, the nucleic acid sequence encoding or comprising the linker connects each of the two or more nucleic acid sequences encoding or comprising the siRNA capable of binding to the target mRNA. In some embodiments, the linker comprises a tRNA linker. In some embodiments, each of the two or more nucleic acid sequences encodes or comprises an siRNA capable of binding to a same target mRNA. In some embodiments, each of the two or more nucleic acid sequences encodes or comprises an siRNA capable of binding to a different target mRNA.

In some aspects, provided herein, is a composition comprising a recombinant polynucleic acid construct encoding or comprising: (i) at least one siRNA capable of specifically binding to IL-6 mRNA; and (ii) an mRNA encoding Interferon beta (IFN-beta). In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6 mRNA. In related aspects, the composition comprises or encodes 3 siRNAs, each directed to IL-6 mRNA. In related aspects, each of the at least 3 siRNAs is the same, different, or a combination thereof. In related aspects, the recombinant polynucleic acid construct comprises a sequence as set forth in SEQ ID NO: 29 or 30 (Compound B1 or B2).

In some aspects, provided herein, is a composition comprising a recombinant polynucleic acid construct encoding or comprising: (i) at least one siRNA capable of specifically binding to Interleukin 6R (IL-6R) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R mRNA. In related aspects, the composition comprises or encodes 3 siRNAs, each directed to IL-6R mRNA. In related aspects, each of the at least 3 siRNAs is the same, different, or a combination thereof. In related aspects, the recombinant polynucleic acid construct comprises a sequence as set forth in SEQ ID NO: 31 (Compound B3).

In some aspects, provided herein, is a composition comprising a recombinant polynucleic acid construct encoding or comprising (i) at least one siRNA capable of specifically binding to Interleukin 6R alpha (IL-6R-alpha) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R-alpha mRNA. In related aspects, the composition comprises or encodes 3 siRNAs, each directed to IL-6R-alpha mRNA. In related aspects, each of the at least 3 siRNAs is the same, different, or a combination thereof. In related aspects, the recombinant polynucleic acid construct comprises a sequence as set forth in SEQ ID NO: 32 (Compound B4).

In some aspects, provided herein, is a composition comprising a recombinant polynucleic acid construct encoding or comprising (i) at least one siRNA capable of specifically binding to Interleukin 6R beta (IL-6R-beta) mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to IL-6R-beta mRNA. In related aspects, the composition comprises or encodes 3 siRNAs, each directed to IL-6R-beta mRNA. In related aspects, each of the at least 3 siRNAs is the same, different, or a combination thereof. In related aspects, the recombinant polynucleic acid construct comprises a sequence as set forth in SEQ ID NO: 33 (Compound B5).

In some aspects, provided herein, is a composition comprising a recombinant polynucleic acid construct encoding or comprising (i) at least one siRNA capable of specifically binding to ACE2 mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to ACE2 mRNA. In related aspects, the composition comprises or encodes 3 siRNAs, each directed to ACE2 mRNA. In related aspects, each of the at least 3 siRNAs is the same, different, or a combination thereof. In related aspects, the recombinant polynucleic acid construct comprises a sequence as set forth in SEQ ID NO: 34 or 35 (Compound B6 or B7).

In some aspects, provided herein, is a composition comprising a recombinant polynucleic acid construct: encoding or comprising (i) at least one small interfering RNA (siRNA) capable of specifically binding to SARS CoV-2 ORF1ab mRNA, at least one siRNA capable of specifically binding to SARS CoV-2 S mRNA, at least one siRNA capable of specifically binding to SARS CoV-2 N mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 3 siRNAs, one directed to SARS CoV-2 ORF1ab mRNA, one directed to SARS CoV-2 S mRNA, and one directed to SARS CoV-2 N mRNA. In related aspects, each of the at least 3 siRNAs is the same, different, or a combination thereof. In certain aspects, such a composition, e.g., a composition comprising Compound B8 (SEQ ID NO: 36) is contemplated for use in methods described herein, e.g., for modulating or regulating gene expression in relation to infection with SARS CoV, SARS CoV-2, or both. In related aspects, the recombinant polynucleic acid construct comprises a sequence as set forth in SEQ ID NO: 36.

In some aspects, provided herein, is a composition comprising a recombinant polynucleic acid construct: encoding or comprising (i) at least one siRNA capable of specifically binding to SARS CoV-2 S mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to SARS CoV-2 S mRNA. In related aspects, the composition comprises or encodes 3 siRNAs, each directed to SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 37 or 39 (Compound B9 or B11).

In some aspects, provided herein, is a composition comprising a recombinant polynucleic acid construct encoding or comprising (i) at least one siRNA capable of specifically binding to SARS CoV-2 N mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 1 siRNA directed to SARS CoV-2 N mRNA. In related aspects, the composition comprises or encodes 3 siRNAs, each directed to SARS CoV-2 N mRNA. In related aspects, each of the at least 3 siRNAs is the same, different, or a combination thereof. In related aspects, the recombinant polynucleic acid construct comprises a sequence as set forth in SEQ ID NO: 38 (Compound B10).

In some aspects, provided herein, is a composition comprising a recombinant polynucleic acid construct: encoding or comprising (i) at least one siRNA capable of specifically binding to SARS CoV-2 ORF1ab mRNA; and (ii) an mRNA encoding IFN-beta. In related aspects, the recombinant polynucleic acid construct in (ii) encodes or further encodes the ACE2 soluble receptor. In related aspects, the composition comprises or encodes 1 siRNA directed to SARS CoV-2 ORF1ab mRNA. In related aspects, the composition comprises or encodes 3 siRNAs, each directed to SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS-CoV, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS-CoV. In related aspects, the recombinant polynucleic acid construct comprises a sequence as set forth in any one of SEQ ID NOs: 40, 41 and 42 (Compounds B12, B13, and B14).

In some aspects, provided herein, is a composition comprising a recombinant polynucleic acid construct: encoding or comprising (i) at least one siRNA capable of specifically binding to IL-6 mRNA, at least one siRNA capable of specifically binding to ACE2 mRNA, and at least one siRNA capable of specifically binding to SARS CoV-2 S mRNA; and (ii) an mRNA encoding IFN-beta. In related aspects, the recombinant polynucleic acid construct in (ii) encodes or further encodes the ACE2 soluble receptor. In related aspects, the composition comprises or encodes at least 1, 2, or 3 siRNAs. In related aspects, the composition comprises or encodes 3 siRNAs, one directed IL-6 mRNA, one directed to ACE2 mRNA, and one directed to SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the recombinant polynucleic acid construct comprises a sequence as set forth in any one of SEQ ID NOs: 43, 44, and 45 (Compounds B15, B16, and B17).

In some aspects, provided herein, is a composition comprising a recombinant polynucleic acid construct encoding or comprising (i) at least one small interfering RNA capable of specifically binding to SARS CoV-2 ORF1ab mRNA, at least one siRNA capable of specifically binding to SARS CoV-2 S mRNA, and at least one siRNA capable of specifically binding to SARS CoV-2 N mRNA; and (ii) an mRNA encoding the ACE2 soluble receptor. In related aspects, the composition comprises or encodes at least 1, 2, or 3 siRNAs. In related aspects, the composition comprises or encodes 3 siRNAs, one directed to ORF1ab mRNA, one directed to SARS CoV-2 S mRNA, and one directed to SARS CoV-2 N mRNA. In related aspects, the recombinant polynucleic acid construct comprises a sequence as set forth in SEQ ID NO: 46 (Compound B18). In related aspects, the recombinant polynucleic acid construct comprises a sequence as set forth in SEQ ID NO: 190 (Compound B18).

In some aspects, provided herein, is a composition comprising a recombinant polynucleic acid construct: encoding or comprising (i) at least one siRNA capable of specifically binding to SARS CoV-2 S mRNA; and (ii) an mRNA encoding the ACE2 soluble receptor. In related aspects, the composition comprises or encodes at least 1, 2, or 3 siRNAs. In related aspects, the composition comprises or encodes 1 siRNA directed to SARS CoV-2 S mRNA. In related aspects, the composition comprises or encodes 3 siRNAs, each directed to SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 47 (Compound B19).

In some aspects, the IFN-beta construct comprises a modified signal peptide as described herein. In some aspects, the present invention provides a composition comprising a recombinant polynucleic acid construct comprising a nucleic acid sequence selected from the group consisting of SEQ ID NOs: 29-47. In some aspects, the present invention provides a composition comprising a recombinant polynucleic acid construct comprising a nucleic acid sequence set forth in SEQ ID NO: 190. In some aspects, the composition comprising the recombinant polynucleic acid construct is useful in the treatment of a viral infection, disease or condition. In some aspects, the composition is present or administered in an amount sufficient to treat or prevent a viral infection, disease or condition.

In some embodiments, the present invention provides a composition and related methods, wherein the composition comprises a recombinant polynucleic acid construct encoding or comprising: at least one siRNA capable of binding to a target RNA; and an mRNA encoding a gene of interest; wherein: the siRNA targets an RNA selected from: an IL-8 mRNA, an IL-1 beta mRNA, an IL-17 mRNA, a TNF-alpha mRNA, a SARS CoV-2 ORF1ab RNA (polyprotein PP1ab, e.g., in a noncoding region or where it encodes a protein that is selected from: a SARS CoV-2 nonstructure protein (NSP), Nsp1, Nsp3 (Nsp3b, Nsp3c, PLpro, and Nsp3e), Nsp7 Nsp8 complex, Nsp9-Nsp10, and Nsp14-Nsp16, 3CLpro, E-channel (E protein), ORF7a, C-terminal RNA binding domain (CRBD), N-terminal RNA binding domain (NRBD), helicase, and RdRp), a SARS CoV-2 Spike protein (S) mRNA, a SARS CoV-2 Nucleocapsid protein (N) mRNA, a tumor necrosis factor alpha (TNF-alpha) mRNA, an interleukin mRNA (including but not limited to interleukin 1 (e.g., IL-1alpha, IL-1beta), interleukin 6 (IL-6), interleukin 6R (IL-6R), interleukin 6R alpha (IL-6R-alpha), interleukin 6R beta (IL-6R-beta), interleukin 18 (IL-18), interleukin 36-alpha (IL-36-alpha), interleukin 36-beta (IL-36-beta), interleukin 36-gamma (IL-36-gamma), interleukin 33 (IL-33)), an Angiotensin Converting Enzyme-2 (ACE2) mRNA, a transmembrane protease, serine 2 (TMPRSS2) mRNA, and a coding NSP12 and 13 RNA; and the gene of interest encodes a protein selected from: IGF-1, IL-4, IGF-1 (including derivatives thereof as described elsewhere herein), carboxypeptidases (e.g., ACE, ACE2, CNDP1, CPA1, CPA2, CPA4, CPA5, CPA6, CPB1, CPB2, CPE, CPN1, CPQ, CPXM1, CPZ, SCPEP1); cytokines (e.g., BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2); extracellular ligands and transporters (e.g., APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, VWC2L); extracellular matrix proteins (e.g., ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLN5, FLRT1, FLRT2, FLRT3, FREM1, GLDN, IBSP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR, TNXB); glucosidases (AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACA5, SPACA5B); glycosyltransferases (e.g., ARTS, B4GALT1, EXTL2, GALNT1, GALNT2, GLT1D1, MGAT4A, ST3GAL1, ST3GAL2, ST3GAL3, ST3GAL4, ST6GAL1, XYLT1); growth factors (e.g., AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF-1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, WISP3); growth factor binding proteins (e.g., CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1); heparin binding proteins (e.g., ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, VTN); hormones (e.g., ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGB5, CGB8, COPA, CORT, CRH, CSH1, CSH2, CSHL1, ENHO, EPO, ERFE, FBN1, FNDC5, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP); hydrolases (e.g., AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPP5, ENTPD5, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4); immunoglobulins (e.g., IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, IGLC3); isomerases (e.g., NAXE, PPIA, PTGDS); kinases (e.g., ADCK1, ADPGK, FAM20C, ICOS, PKDCC); lyases (e.g., PM20D1, PAM, CA6); metalloenzyme inhibitors (e.g., FETUB, SPOCK3, TIMP2, TIMP3, TIMP4, WFIKKN1, WFIKKN2); metalloproteases (e.g., ADAM12, ADAM28, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMP1, MMP8, MMP9, PAPPA, PAPPA2, TLL1, TLL2); milk proteins (e.g., CSN1S1, CSN2, CSN3, LALBA); neuroactive proteins (e.g., CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3); proteases (e.g., ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSK5, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, TPSD1); protease inhibitors (e.g., A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINB5, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, WFDC8); protein phosphatases (e.g., ACP7, ACPP, PTEN, PTPRZ1); esterases (e.g., BCHE, CEL, CES4A, CES5A, NOTUM, SIAE); transferases (e.g., METTL24, FKRP, CHSY1, CHST9, B3GAT1); vasoactive proteins (e.g., AGGF1, AGT, ANGPT1, ANGPT2, ANGPTL4, ANGPTL6, EDN1, EDN2, EDN3, NTS), a Type I interferon (e.g., an IFN-α, including, but not limited to an interferon alpha-n3, an interferon alpha-2a, and an interferon alpha-2b, an IFN-β, an IFN-δ, an IFN-ε, an IFN-κ, an IFN-ν, an IFN-τ, and an IFN-ω), a Type II interferon (e.g., IFN-γ), a Type III interferon (e.g., IFN-λ) an interleukin, e.g., IL-37, IL-38, and a soluble ACE2 receptor. In some aspects, the composition comprising the recombinant polynucleic acid construct is useful in the treatment of a viral infection, disease or condition. In some aspects, the composition is present or administered in an amount sufficient to treat or prevent a viral infection, disease or condition. In some embodiments, the disease or the condition is selected from the group consisting of intervertebral disc disease (IVDD), osteoarthritis, and psoriasis.

### Recombinant RNA Construct for Treating a Viral Disease or Condition

As described above, in some aspects, the recombinant polynucleic acid construct is a recombinant RNA construct. In some aspects, the recombinant polynucleic acid construct or recombinant RNA construct is useful in a composition for treating or preventing a viral infection, disease, or condition. In some aspects, the invention provides a composition comprising a recombinant RNA construct comprising: (i) a small interfering RNA (siRNA) capable of binding to a target RNA (e.g., mRNA); and (ii) an mRNA of a gene of interest; wherein the target mRNA is different from the mRNA encoding the gene of interest.

In some embodiments, the recombinant RNA construct comprises 1-10 siRNA species. In some embodiments, the siRNA species are the same, e.g., capable of binding to the same target mRNA. In some embodiments, the siRNA species are different, e.g., capable of binding to different target mRNAs. In some embodiments, some siRNA species are the same and some are different. In some embodiments, the siRNA comprises a sense siRNA strand. In some embodiments, the siRNA comprises an anti-sense siRNA strand. In some embodiments, the siRNA comprises a sense and an anti-sense siRNA strand. In some embodiments, the siRNA does not affect the expression of the gene of interest. In some embodiments, the siRNA does not inhibit the expression of the gene of interest. In some embodiments, the recombinant RNA construct comprises two or more nucleic acid sequences comprising an siRNA capable of binding to a target mRNA. In some embodiments, the recombinant RNA construct further comprises or encodes a linker. In some embodiments, the nucleic acid sequence encoding or comprising the linker connects each of the two or more nucleic acid sequences comprising the siRNA capable of binding to the target mRNA. In some embodiments, the linker comprises a tRNA linker. In some embodiments, the linker comprises a 2A peptide linker. In some embodiments, each of the two or more nucleic acid sequences comprises an siRNA capable of binding to a same target mRNA. In some embodiments, each of the two or more nucleic acid sequences comprises an siRNA capable of binding to a different target mRNA.

In some embodiments, the expression of the target mRNA is modulated by the siRNA capable of binding to the target mRNA. In some embodiments, the expression of the target mRNA is downregulated by the siRNA capable of specifically binding to the target mRNA.

In some embodiments, the recombinant RNA construct comprises a nucleic acid sequence comprising a gene of interest (and thereby encoding an mRNA of interest and/or a protein of interest corresponding to the gene of interest). In some embodiments, the recombinant RNA construct comprises two or more nucleic acid sequences, each comprising a gene of interest and thereby each encoding an mRNA of interest and/or a protein of interest corresponding to the gene.

In some embodiments, each of the two or more nucleic acid sequences comprises the same gene of interest. In some embodiments, each of the two or more nucleic acid sequences encodes the same mRNA and/or protein of interest. In some embodiments, the recombinant RNA construct comprises three or more nucleic acid sequences, each comprising a gene of interest and thereby each encoding an mRNA of interest and/or a protein of interest corresponding to the gene. In some embodiments, each of the three or more nucleic acid sequences can comprise the same gene of interest, encode the same mRNA of interest, and/or encode the same protein of interest. In some embodiments, each of the three or more nucleic acid sequences can comprise different genes of interest, encode different mRNAs of interest, and/or encode different proteins of interest. In some embodiments, two or more of the three or more nucleic acid sequences can comprise the same gene of interest, encode the same mRNA of interest, and/or encode the same protein of interest, while one or more of the three or more nucleic acid sequences comprises a different gene of interest, encodes a different mRNA of interest, and/or encodes a different protein of interest from the two or more of the three or more nucleic acid sequences.

In some embodiments, the expression level of the gene or protein of interest is modulated by expressing an mRNA or a protein encoded by the gene of interest. In some embodiments, the expression level of the gene of interest is upregulated by expressing an mRNA or a protein encoded by the gene of interest. In some embodiments, the recombinant RNA construct is codon-optimized. In some embodiments, the recombinant RNA construct is not codon-optimized.

In some embodiments, the recombinant RNA construct further comprises a nucleic acid sequence encoding a target motif, also referred to as a targeting motif. In some embodiments, the nucleic acid sequence encoding the target motif is operably linked to the at least one nucleic acid sequence encoding the gene of interest. In some embodiments, the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS). In some embodiments, the target motif is selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid.

In some embodiments, the signal peptide is selected from the group consisting of (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide homologous to a protein encoded by the gene of interest, wherein the signal peptide homologous to the protein encoded by the gene of interest is modified by insertion, deletion, and/or substitution of at least one amino acid; and (d) a naturally occurring amino acid sequence which does not have the function of a signal peptide in nature, wherein the naturally occurring amino acid sequence is optionally modified by insertion, deletion, and/or substitution of at least one amino acid. In some embodiments, the amino acids 1-9 of the N-terminal end of the signal peptide have an average hydrophobic score of above 2.

In some aspects, provided herein, is a cell comprising the composition of any recombinant polynucleic acid or RNA construct described herein. In some aspects, provided herein, is a pharmaceutical composition comprising the composition of any recombinant polynucleic acid or RNA construct described herein and a pharmaceutically acceptable excipient. In some aspects, provided herein, is a method of treating a disease or a condition in a subject in need thereof, comprising administering to the subject the pharmaceutical composition described herein. In some embodiments, the disease or condition is COVID-19. In some embodiments, the disease or condition is SARS (severe acute respiratory syndrome) caused by infection with SARS-CoV-1 or SARS-CoV-2. In some embodiments, the subject is a mammal. In some embodiments, the subject is a human. In some embodiments, the subject is an adult, a child, or an infant. In some embodiments, the subject is a companion animal. In some embodiments, the subject is feline, canine, or a rodent. In some embodiments, the subject is a dog or a cat.

In some aspects, provided herein, is a method of simultaneously expressing an siRNA and an mRNA from a single RNA transcript in a cell, comprising introducing into the cell the composition of any recombinant polynucleic acid or RNA construct described herein. In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence of a gene of interest; wherein the target mRNA is different from an mRNA encoded by the gene of interest, and wherein the expression of the target mRNA and the gene of interest is modulated simultaneously.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence of a gene of interest; wherein the target mRNA is different from an mRNA encoded by the gene of interest, and wherein the expression of the target mRNA is downregulated and the expression of the gene of interest is upregulated simultaneously. In some embodiments, the expression of the target mRNA is downregulated by the siRNA capable of binding to the target mRNA. In some embodiments, the expression of the gene of interest is upregulated by expressing an mRNA or a protein encoded by the gene of interest.

In some aspects, provided herein, is a method of producing an RNA construct comprising a small interfering RNA (siRNA) capable of binding to a target messenger RNA (mRNA), and an mRNA of a gene of interest, wherein the target mRNA is different from the mRNA encoding the gene of interest, the method comprising: (a) providing, for in vitro transcription reaction: (i) a polynucleic acid construct comprising a promoter, at least one nucleic acid sequence encoding an siRNA capable of binding to a target mRNA, at least one nucleic acid sequence comprising a gene of interest, and a nucleic acid sequence encoding a poly(A) tail; (ii) an RNA polymerase; and (iii) a mixture of nucleotide triphosphates (NTPs); and (b) isolating and purifying transcribed RNAs from the in vitro transcription reaction mixture, thus producing the RNA construct. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase. In some embodiments, the RNA polymerase is T7 RNA polymerase. In some embodiments, the mixture of NTPs comprises unmodified NTPs. In some embodiments, the mixture of NTPs comprises modified NTPs. In some embodiments, the modified NTPs comprise N¹-methylpseudouridine, Pseudouridine, N¹-Ethylpseudouridine, N¹-Methoxymethylpseudouridine, N¹-Propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-Bromouridine, 5-lodouridine, 5,6-dihydrouridine, 6-Azauridine, Thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxycytidine, 5-hydroxycytidine, 5-lodocytidine, 5-Bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-Aminoadenosine, N⁶-isopentenyladenosine, N⁶,N⁶-dimethyladenosine, 7-methyladenine, 2-methylthio-adenine, and 2-methoxy-adenine.

In some embodiments, step (a) further comprises providing a capping enzyme. In some embodiments, isolating and purifying transcribed RNAs comprise column purification.

In some embodiments, specific binding of an siRNA to its mRNA target results in interference with the normal function of the target mRNA to cause a modulation, e.g., downregulation, of function and/or activity, and wherein there is a sufficient degree of complementarity to avoid non-specific binding of the siRNA to non-target nucleic acid sequences under conditions in which specific binding is desired, i.e., under physiological conditions in the case of in vivo assays or therapeutic treatment, and under conditions in which assays are performed in the case of in vitro assays.

In some aspects, provided herein, is a composition comprising a recombinant RNA construct comprising: (i) at least one siRNA capable of specifically binding to IL-6 mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to IL-6 mRNA. In related aspects, the composition comprises 3 siRNAs, each directed to IL-6 mRNA. In related aspects, each of the at least 3 siRNAs is the same, different, or a combination thereof. In related aspects, the recombinant RNA construct comprises a sequence encoded by a sequence as set forth in SEQ ID NO: 29 or 30 (Compound B1 or B2).

In some aspects, provided herein, is a composition comprising a recombinant RNA construct comprising: (i) at least one siRNA capable of specifically binding to Interleukin 6R (IL-6R) mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to IL-6R mRNA. In related aspects, the composition comprises 3 siRNAs, each directed to IL-6R mRNA. In related aspects, each of the at least 3 siRNAs is the same, different, or a combination thereof. In related aspects, the recombinant RNA construct comprises a sequence encoded by the sequence as set forth in SEQ ID NO: 31 (Compound B3).

In some aspects, provided herein, is a composition comprising a recombinant RNA construct comprising: (i) at least one siRNA capable of specifically binding to Interleukin 6R alpha (IL-6R-alpha) mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to IL-6R-alpha mRNA. In related aspects, the composition comprises 3 siRNAs, each directed to IL-6R-alpha mRNA. In related aspects, each of the at least 3 siRNAs is the same, different, or a combination thereof. In related aspects, the recombinant RNA construct comprises a sequence encoded by the sequence as set forth in SEQ ID NO: 32 (Compound B4).

In some aspects, provided herein, is a composition comprising a recombinant RNA construct comprising: (i) at least one siRNA capable of specifically binding to Interleukin 6R beta (IL-6R-beta) mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to IL-6R-beta mRNA. In related aspects, the composition comprises 3 siRNAs, each directed to IL-6R-beta mRNA. In related aspects, each of the at least 3 siRNAs is the same, different, or a combination thereof. In related aspects, the recombinant RNA construct comprises a sequence encoded by the sequence as set forth in SEQ ID NO: 33 (Compound B5).

In some aspects, provided herein, is a composition comprising a recombinant RNA construct comprising: (i) at least one siRNA capable of specifically binding to ACE2 mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to ACE2 mRNA. In related aspects, the composition comprises 3 siRNAs, each directed to ACE2 mRNA. In related aspects, each of the at least 3 siRNAs is the same, different, or a combination thereof. In related aspects, the recombinant RNA construct comprises a sequence encoded by a sequence as set forth in SEQ ID NO: 34 or 35 (Compound B6 or B7).

In some aspects, provided herein, is a composition comprising a recombinant RNA construct comprising: (i) at least one small interfering RNA (siRNA) capable of specifically binding to SARS CoV-2 ORF1ab mRNA, at least one siRNA capable of specifically binding to SARS CoV-2 S mRNA, at least one siRNA capable of specifically binding to SARS CoV-2 N mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 3 siRNAs, one directed to SARS CoV-2 ORF1ab mRNA, one directed to SARS CoV-2 S mRNA, and one directed to SARS CoV-2 N mRNA. In related aspects, each of the at least 3 siRNAs is the same, different, or a combination thereof. In certain aspects, such a composition, e.g., a composition comprising Compound B8 (SEQ ID NO: 36) is contemplated for use in methods described herein, e.g., for modulating or regulating gene expression in relation to infection with SARS CoV, SARS CoV-2, or both. In related aspects, the recombinant RNA construct comprises a sequence encoded by the sequence as set forth in SEQ ID NO: 36.

In some aspects, provided herein, is a composition comprising a recombinant RNA construct comprising: (i) at least one siRNA capable of specifically binding to SARS CoV-2 S mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to SARS CoV-2 S mRNA. In related aspects, the composition comprises 3 siRNAs, each directed to SARS CoV-2 S mRNA. In related aspects, each of the at least 3 siRNAs is the same, different, or a combination thereof. In related aspects, the recombinant RNA construct comprises a sequence encoded by a sequence as set forth in SEQ ID NO: 37 or 39 (Compound B9 or B11).

In some aspects, provided herein, is a composition comprising a recombinant RNA construct comprising: (i) at least one siRNA capable of specifically binding to SARS CoV-2 N mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 1 siRNA directed to SARS CoV-2 N mRNA. In related aspects, the composition comprises 3 siRNAs, each directed to SARS CoV-2 N mRNA. In related aspects, each of the at least 3 siRNAs is the same, different, or a combination thereof. In related aspects, the recombinant RNA construct comprises a sequence encoded by the sequence as set forth in SEQ ID NO: 38 (Compound B10).

In some aspects, provided herein, is a composition comprising a recombinant RNA construct comprising: (i) at least one siRNA capable of specifically binding to SARS CoV-2 ORF1ab mRNA; and (ii) an mRNA encoding IFN-beta. In related aspects, the mRNA of ii) encodes or further encodes an ACE2 soluble receptor. In related aspects, the composition comprises 1 siRNA directed to SARS CoV-2 ORF1ab mRNA. In related aspects, the composition comprises 3 siRNAs, each directed to SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS. In related aspects, the recombinant RNA construct comprises a sequence encoded by a sequence as set forth in any one of SEQ ID NOs: 40, 41 and 42 (Compounds B12, B13 and B14).

In some aspects, provided herein, is a composition comprising a recombinant RNA construct comprising: (i) at least one siRNA capable of specifically binding to IL-6 mRNA, at least one siRNA capable of specifically binding to ACE2 mRNA, and at least one siRNA capable of specifically binding to SARS CoV-2 S mRNA; and (ii) an mRNA encoding IFN-beta. In related aspects, the mRNA of ii) encodes or further encodes an ACE2 soluble receptor. In related aspects, the composition comprises at least 1, 2, or 3 siRNAs. In related aspects, the composition comprises 3 siRNAs, one directed IL-6 mRNA, one directed to ACE2 mRNA, and one directed to SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOS: 52 and 54, respectively). In related aspects, the recombinant RNA construct comprises a sequence encoded by a sequence as set forth in any one of SEQ ID NOS: 43, 44, and 45 (Compounds B15, B16, and B17).

In some aspects, provided herein, is a composition comprising a recombinant RNA construct comprising: (i) at least one small interfering RNA capable of specifically binding to SARS CoV-2 ORF1ab mRNA, at least one siRNA capable of specifically binding to SARS CoV-2 S mRNA, and at least one siRNA capable of specifically binding to SARS CoV-2 N mRNA; and (ii) an mRNA encoding an ACE2 soluble receptor. In related aspects, the composition comprises at least 1, 2, or 3 siRNAs. In related aspects, the composition comprises 3 siRNAs, one directed to ORF1ab mRNA, one directed to SARS CoV-2 S mRNA, and one directed to SARS CoV-2 N mRNA. In related aspects, the recombinant RNA construct comprises a sequence encoded by the sequence as set forth in SEQ ID NO: 46 (Compound B18). In related aspects, the recombinant RNA construct comprises a sequence as set forth in SEQ ID NO: 190.

In some aspects, provided herein, is a composition comprising a recombinant RNA construct comprising: (i) at least one siRNA capable of specifically binding to SARS CoV-2 S mRNA; and (ii) an mRNA encoding ACE2 soluble receptor. In related aspects, the composition comprises at least 1, 2, or 3 siRNAs. In related aspects, the composition comprises 1 siRNA directed to SARS CoV-2 S mRNA. In related aspects, the composition comprises 3 siRNAs, each directed to SARS CoV-2 S mRNA. In related aspects, each of the at least 3 siRNAs is the same, different, or a combination thereof. In related aspects, the recombinant RNA construct comprises a sequence encoded by the sequence as set forth in SEQ ID NO: 47 (Compound B19).

In some aspects, the IFN-beta construct comprises a modified signal peptide as described herein.

In some aspects, the present invention provides a composition comprising a recombinant RNA construct comprising a nucleic acid sequence encoded by a sequence selected from the group consisting of SEQ ID NOs: 29-47. In some aspects, the present invention provides a composition comprising a recombinant RNA construct comprising a nucleic acid sequence as set forth in SEQ ID NO: 190.

In some embodiments, the present invention provides a composition and related methods, wherein the composition comprises a recombinant RNA construct comprising: at least one siRNA capable of binding to a target RNA; and an mRNA encoding a gene of interest; wherein: the siRNA targets an RNA selected from: an IL-8 mRNA, an IL-1 beta mRNA, an IL-17 mRNA, a TNF-alpha mRNA, a SARS CoV-2 ORF1ab RNA (polyprotein PP1ab, e.g., in a noncoding region or where it encodes a protein that is selected from: a SARS CoV-2 nonstructure protein (NSP), Nsp1, Nsp3 (Nsp3b, Nsp3c, PLpro, and Nsp3e), Nsp7 Nsp8 complex, Nsp9-Nsp10, and Nsp14-Nsp16, 3CLpro, E-channel (E protein), ORF7a, C-terminal RNA binding domain (CRBD), N-terminal RNA binding domain (NRBD), helicase, and RdRp), a SARS CoV-2 Spike protein (S) mRNA, a SARS CoV-2 Nucleocapsid protein (N) mRNA, a tumor necrosis factor alpha (TNF-alpha) mRNA, an interleukin mRNA (including but not limited to interleukin 1 (e.g., IL-1alpha, IL-1beta), interleukin 6 (IL-6), interleukin 6R (IL-6R), interleukin 6R alpha (IL-6R-alpha), interleukin 6R beta (IL-6R-beta), interleukin 18 (IL-18), interleukin 36-alpha (IL-36-alpha), interleukin 36-beta (IL-36-beta), interleukin 36-gamma (IL-36-gamma), interleukin 33 (IL-33)), an Angiotensin Converting Enzyme-2 (ACE2) mRNA, a transmembrane protease, serine 2 (TMPRSS2) mRNA, and a coding NSP12 and 13 RNA; and the gene of interest encodes a protein selected from: IGF-1, IL-4, IGF-1 (including derivatives thereof as described elsewhere herein), carboxypeptidases (e.g., ACE, ACE2, CNDP1, CPA1, CPA2, CPA4, CPA5, CPA6, CPB1, CPB2, CPE, CPN1, CPQ, CPXM1, CPZ, SCPEP1); cytokines (e.g., BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2); extracellular ligands and transporters (e.g., APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, VWC2L); extracellular matrix proteins (e.g., ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLN5, FLRT1, FLRT2, FLRT3, FREM1, GLDN, IBSP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR, TNXB); glucosidases (AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACA5, SPACA5B); glycosyltransferases (e.g., ARTS, B4GALT1, EXTL2, GALNT1, GALNT2, GLT1D1, MGAT4A, ST3GAL1, ST3GAL2, ST3GAL3, ST3GAL4, ST6GAL1, XYLT1); growth factors (e.g., AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF-1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, WISP3); growth factor binding proteins (e.g., CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1); heparin binding proteins (e.g., ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, VTN); hormones (e.g., ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGB5, CGB8, COPA, CORT, CRH, CSH1, CSH2, CSHL1, ENHO, EPO, ERFE, FBN1, FNDC5, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP); hydrolases (e.g., AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPP5, ENTPD5, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4); immunoglobulins (e.g., IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, IGLC3); isomerases (e.g., NAXE, PPIA, PTGDS); kinases (e.g., ADCK1, ADPGK, FAM20C, ICOS, PKDCC); lyases (e.g., PM20D1, PAM, CA6); metalloenzyme inhibitors (e.g., FETUB, SPOCK3, TIMP2, TIMP3, TIMP4, WFIKKN1, WFIKKN2); metalloproteases (e.g., ADAM12, ADAM28, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMP1, MMP8, MMP9, PAPPA, PAPPA2, TLL1, TLL2); milk proteins (e.g., CSN1S1, CSN2, CSN3, LALBA); neuroactive proteins (e.g., CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3); proteases (e.g., ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSK5, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, TPSD1); protease inhibitors (e.g., A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINB5, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, WFDC8); protein phosphatases (e.g., ACP7, ACPP, PTEN, PTPRZ1); esterases (e.g., BCHE, CEL, CES4A, CES5A, NOTUM, SIAE); transferases (e.g., METTL24, FKRP, CHSY1, CHST9, B3GAT1); vasoactive proteins (e.g., AGGF1, AGT, ANGPT1, ANGPT2, ANGPTL4, ANGPTL6, EDN1, EDN2, EDN3, NTS), a Type I interferon (e.g., an IFN-α, including, but not limited to an interferon alpha-n3, an interferon alpha-2a, and an interferon alpha-2b, an IFN-β, an IFN-δ, an IFN-ε, an IFN-κ, an IFN-ν, an IFN-τ, and an IFN-ω), a Type II interferon (e.g., IFN-γ), a Type III interferon (e.g., IFN-λ) an interleukin, e.g., IL-37, IL-38, and a soluble ACE2 receptor. In some aspects, the composition comprising the recombinant RNA construct is useful in the treatment of a viral infection, disease or condition. In some aspects, the composition is present or administered in an amount sufficient to treat or prevent a viral infection, disease or condition. In some embodiments, the disease or the condition is selected from the group consisting of intervertebral disc disease (IVDD), osteoarthritis, and psoriasis.

In some aspects, the composition comprising the recombinant RNA construct is useful in the treatment of a skin disease or condition. In some aspects, the composition is present or administered in an amount sufficient to treat or prevent a skin disease or condition. In some embodiments, the skin disease or condition comprises an inflammatory skin disorder. In some embodiments, the inflammatory skin disorder comprises psoriasis. In some aspects, the composition comprising the recombinant RNA construct is useful in the treatment of a muscular disease or condition. In some aspects, the composition is present or administered in an amount sufficient to treat or prevent a muscular disease or condition. In some embodiments, the muscular disease or condition comprises a skeletal muscle disorder. In some embodiments, the skeletal muscle disorder comprises fibrodysplasia ossificans progressiva (FOP). In some aspects, the composition comprising the recombinant RNA construct is useful in the treatment of a neurodegenerative disease or condition. In some aspects, the composition is present or administered in an amount sufficient to treat or prevent a neurodegenerative disease or condition. In some embodiments, the neurodegenerative disease or condition comprises a motor neuron disorder. In some embodiments, the motor neuron disorder comprises amyotrophic lateral sclerosis (ALS). In some aspects, the composition comprising the recombinant RNA construct is useful in the treatment of a joint disease or condition. In some aspects, the composition is present or administered in an amount sufficient to treat or prevent a joint disease or condition. In some embodiments, the joint disease or condition comprises a joint degeneration. In some embodiments, the joint degeneration comprises intervertebral disc disease (IVDD) or osteoarthritis (OA).

RNA Interference and Small Interfering RNA (siRNA)

RNA interference (RNAi) or RNA silencing is a process in which RNA molecules inhibit gene expression or translation, by neutralizing target mRNA molecules. RNAi process is described in Mello & Conte (2004) Nature 431, 338-342, Meister & Tuschl (2004) Nature 431, 343-349, Hannon & Rossi (2004) Nature 431, 371-378, and Fire (2007) Angew. Chem. Int. Ed. 46, 6966-6984. Briefly, in a natural process, the reaction initiates with a cleavage of long double-stranded RNA (dsRNA) into small dsRNA fragments or siRNAs with a hairpin or loop structure by a dsRNA-specific endonuclease Dicer. These small dsRNA fragments or siRNAs are then integrated into RNA-induced silencing complex (RISC) and guide the RISC to the target mRNA sequence. During interference, the siRNA duplex unwinds, and the antisense strand remains in complex with RISC to lead RISC to the target mRNA sequence to induce degradation and subsequent suppression of protein translation. Unlike commercially available synthetic siRNA (e.g., Patisiran, etc.), the siRNA in the present invention utilizes endogenous Dicer and RISC pathway in the cytoplasm of a cell to get cleaved from mRNA transcript construct of the present invention and follow the natural process detailed above. In addition, as the rest of the mRNA transcript of the present invention is left intact after cleavage of the siRNA by Dicer, and the desired protein expression from the gene of interest in the mRNA transcript of the present invention is attained.

In some aspects, provided herein, is a composition comprising a recombinant polynucleic acid or RNA construct comprising at least one nucleic acid sequence encoding or comprising a siRNA capable of binding to a target mRNA. In some embodiments, the recombinant polynucleic acid or RNA construct comprises a nucleic acid sequence encoding or comprising a sense siRNA strand. In some embodiment, the recombinant polynucleic acid or RNA construct comprises a nucleic acid sequence encoding or comprising an anti-sense siRNA strand. In a preferred embodiment, the recombinant polynucleic acid or RNA construct comprises a nucleic acid sequence encoding or comprising a sense siRNA strand and a nucleic acid sequence encoding or comprising an anti-sense siRNA strand. The details of siRNA comprised in the present invention is described in Cheng, et al. (2018) J. Mater. Chem. B., 6, 4638-4644, which is incorporated by reference herein.

In some embodiments, the recombinant polynucleic acid or RNA construct has at least 1 copy of siRNA, i.e., a nucleic acid sequence encoding or comprising sense strand of siRNA and a nucleic acid sequence encoding or comprising anti-strand of siRNA. 1 copy of siRNA, as described herein, can refer to 1 copy of sense strand siRNA and 1 copy of anti-sense strand siRNA. In some embodiments, the recombinant polynucleic acid or RNA construct has more than 1 copy of siRNA, i.e., more than 1 copy of nucleic acid sequence encoding or comprising sense strand of siRNA and more than 1 copy of nucleic acid sequence encoding or comprising anti-strand of siRNA. In some embodiments, the recombinant polynucleic acid or RNA construct has 1 to 10 copies of siRNA, i.e., 1 to 10 copies of nucleic acid sequence encoding or comprising sense strand of siRNA and 1 to 10 copies of nucleic acid sequence encoding or comprising anti-strand of siRNA. In some embodiments, the recombinant polynucleic acid or RNA construct has 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 copies of siRNA. In some embodiments, the recombinant polynucleic acid or RNA construct has 1, 2, 3, 4, 5, 6, 7, 8, 9, or 10 copies of siRNA. In some embodiments, the recombinant polynucleic acid or RNA construct has at least 1, 2, 3, 4, 5, 6, 7, 8, or 9 copies of siRNA. In some embodiments, the recombinant polynucleic acid or RNA construct has at most 2, 3, 4, 5, 6, 7, 8, 9, or 10 copies of siRNA.

In some embodiments, the recombinant polynucleic acid or RNA construct further comprises a nucleic acid sequence encoding or comprising a linker. In some embodiments, the nucleic acid sequence encoding or comprising the linker may connect each of the two or more nucleic acid sequences encoding the siRNA. In some embodiments, the linker may be a non-cleavable linker. In a preferred embodiment, the linker may be a cleavable linker. In some embodiments, the linker may be a self-cleavable linker. In some embodiments, the linker may be a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising

(SEQ ID NO: 24)
AACAAAGCACCAGTGGTCTAGTGGTAGAATAGTACCCTGCCACGGTA
CAGACCCGGGTTCGATTCCCGGCTGGTGCA.

In some aspects, provided herein, is a composition comprising a recombinant polynucleic acid or RNA construct comprising at least one nucleic acid sequence encoding or comprising a siRNA capable of binding to a target mRNA. A list of non-limiting examples of the target mRNAs that the siRNA is capable of binding to include an mRNA encoding Interleukin 8 (IL-8), Interleukin 1 beta (IL-1 beta), Interleukin 17 (IL-17), and Tumor Necrosis Factor alpha (TNF-alpha, or TNF-α). A list of additional examples of the target RNAs that the siRNA is capable of binding to includes an mRNA encoding Activin receptor-like kinase-2 (ALK2) and Superoxide dismutase-1 (SOD1).

In some aspects, the siRNA is capable of binding to a target RNA that is a coronavirus RNA. In some embodiments, the coronavirus RNA is a target mRNA that encodes a coronavirus protein. In some embodiments, the coronavirus RNA is a target noncoding RNA. In some embodiments, the coronavirus is an Alphacoronavirus, Betacoronavirus, Gammacoronavirus or a Deltacoronavirus. In some embodiments, the coronavirus target mRNA encodes a protein selected from: SARS CoV-2 ORF1ab (polyprotein PP1ab); SARS CoV-2 Spike protein (S), and SARS CoV-2 Nucleocapsid protein (N). In some embodiments, the siRNA is capable of binding to an ORF1ab mRNA in a region or where it encodes a protein that is selected from: a SARS CoV-2 nonstructure protein (NSP), Nsp1, Nsp3 (Nsp3b, Nsp3c, PLpro, and Nsp3e), Nsp7 Nsp8 complex, Nsp9-Nsp10, and Nsp14-Nsp16, 3CLpro, E-channel (E protein), ORF7a, C-terminal RNA binding domain (CRBD), N-terminal RNA binding domain (NRBD), helicase, and RdRp. In some embodiments, the target coding RNA is SARS CoV-2 NSP12 and 13. In some embodiments, the target mRNA encodes a coronavirus protein that is conserved among coronaviruses, e.g., among SARS-CoV, SARS-CoV-2, and/or MERS-CoV, and the corresponding siRNA is useful in compositions and methods that can be used to treat two or more different diseases or conditions, e.g., two or more diseases or conditions caused by or associated with more than one coronavirus. In some embodiments, the target mRNA encodes SARS-CoV-2 Nsp15, which is 89% identical to the analogous protein of SARS-CoV, and the polynucleic acid construct can be used to treat SARS-CoV and SARS-CoV-2 infection. In some embodiments, the siRNA is capable of binding to an mRNA target or noncoding RNA target common to more than one coronavirus. In some embodiments, the coding RNA target is Nsp12-Nsp13, relating to SARS CoV-2, SARS-CoV and MERS-CoV. In some embodiments, the coronavirus target RNA and any corresponding encoded protein is any one that is known to those of skill in the art or described in the literature, e.g., by Wu, et al., 27 Feb. 2020, Acta Pharmaceutica Sinica, preproof at doi.org/10.1016/j.apsb.2020.02.008, incorporated by reference herein. In some embodiments, the target mRNA encodes a host protein. In some embodiments, the target mRNA encodes a cytokine. In some embodiments, the target mRNA encodes a cytokine selected from the group consisting of: tumor necrosis factor alpha (TNF-alpha), an interleukin (including but not limited to interleukin 1 (e.g., IL-1alpha, IL-1beta), interleukin 6 (IL-6), interleukin 6R (IL-6R), interleukin 6R alpha (IL-6R-alpha), interleukin 6R beta (IL-6R-beta), interleukin 18 (IL-18), interleukin 36-alpha (IL-36-alpha), interleukin 36-beta (IL-36-beta)), interleukin 36-gamma (IL-36-gamma), and interleukin 33 (IL-33)). The role of TNF-alpha in Covid-19 is discussed in the literature, e.g., by Feldmann, et al., 9 Apr. 2020, The Lancet S0140-6736(20)30858-8, incorporated by reference herein. In some embodiments, the target mRNA encodes an inflammatory cytokine. In some embodiments, the target mRNA encodes a host viral entry protein. In some embodiments, the host viral entry protein is an Angiotensin Converting Enzyme-2 (ACE2). In some embodiments, the target mRNA encodes a host enzyme. In some embodiments, the enzyme is transmembrane protease, serine 2 (TMPRSS2).

In some embodiments, the recombinant nucleic acid construct comprises two or more nucleic acid sequences encoding an siRNA capable of binding to a target RNA. In some embodiments, the target RNA is an mRNA. In some embodiments, the target RNA is a noncoding RNA. In some embodiments, the recombinant nucleic acid construct comprises three nucleic acid sequences encoding an siRNA capable of binding to a target mRNA. In some embodiments, the recombinant nucleic acid construct comprises four nucleic acid sequences encoding an siRNA capable of binding to a target mRNA. In some embodiments, the recombinant nucleic acid construct comprises 5, 6, 7, 8, 9, 10, 11, 12, 13, 14, 15, or more nucleic acid sequences encoding an siRNA capable of binding to a target mRNA. In some embodiments, the recombinant nucleic acid construct comprises 2 to 10 nucleic acid sequences encoding an siRNA capable of binding to a target mRNA. In some embodiments, the recombinant nucleic acid construct comprises 2 to 6 nucleic acid sequences encoding an siRNA capable of binding to a target mRNA. In some embodiments, each of the two or more nucleic acid sequences encodes an siRNA capable of binding to a same target mRNA. In some embodiments, each of the two or more nucleic acid sequences encodes an siRNA capable of binding to a different target mRNA.

In some embodiments, the expression of the target mRNA is modulated by the siRNA capable of binding to the target mRNA. In some embodiments, the siRNA is capable of binding to a target mRNA in its 5′ untranslated region. In some embodiments, the siRNA is capable of binding to a target mRNA in its 3′ untranslated region. In some embodiments, the siRNA is capable of binding to a target mRNA in a translated region. In some embodiments, the expression of the target mRNA is downregulated by the siRNA capable of binding to the target mRNA. In some embodiments, the expression of the target mRNA is inhibited by the siRNA capable of binding to the target mRNA. Inhibition or downregulation of the expression of the target mRNA, as described herein, can refer to, but is not limited to, interference with the target mRNA to interfere with translation of the protein from the target mRNA encoded by or comprised in the recombinant polynucleic acid or RNA construct, respectively; thus, inhibition or downregulation of the expression of the target mRNA can refer to, but is not limited to, a decreased level of the protein expressed from the target mRNA compared to a level of the protein expressed from the target mRNA in the absence of the recombinant polynucleic acid or RNA construct comprising siRNA capable of binding to the target mRNA. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

In some aspects, provided herein, is a composition comprising a recombinant polynucleic acid or RNA construct comprising at least one nucleic acid sequence encoding or comprising a siRNA capable of binding to a target mRNA and at least one nucleic acid sequence encoding a gene of interest wherein the target mRNA is different from an mRNA encoded by the gene of interest. In some embodiments, the siRNA does not affect the expression of the gene of interest. In some embodiments, the siRNA is not capable of binding to the nucleic acid encoding the gene of interest. In a preferred embodiment, the siRNA does not inhibit the expression of the gene of interest. In another preferred embodiment, the siRNA does not downregulate the expression of the gene of interest. Inhibiting or downregulating the expression of the gene of interest, as described herein, can refer to, but is not limited to, interfering with transcription of DNA and/or translation of protein from the recombinant polynucleic acid or RNA construct; thus, inhibiting or downregulating the expression of the gene of interest can refer to, but is not limited to, a decreased level of protein compared to a level of protein expressed in the absence of the recombinant polynucleic acid or RNA construct comprising siRNA capable of binding to the target mRNA. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

In some aspects, the siRNA comprises a sense strand encoded by a sequence selected from SEQ ID NOs: 80-109. In some aspects, the siRNA comprises a sense strand encoded by a sequence selected from SEQ ID NOs: 80-109, and the corresponding antisense strand encoded by a sequence selected from SEQ ID NOS: 110-139. In some embodiments, the target RNA is an IL-8 mRNA, and the siRNA comprises a sense strand encoded by a sequence selected from SEQ ID NOs: 80-83. In some embodiments, the target RNA is an IL-8 mRNA, and the siRNA comprises a sense strand encoded by a sequence selected from SEQ ID NOs: 80-83, and a corresponding antisense strand encoded by a sequence selected from SEQ ID NOs: 110-113, respectively. In some embodiments, the target RNA is an IL-1 beta mRNA, and the siRNA comprises a sense strand encoded by a sequence selected from SEQ ID NOs: 84-86. In some embodiments, the target RNA is an IL-1 beta mRNA, and the siRNA comprises a sense strand encoded by a sequence selected from SEQ ID NOs: 84-86, and a corresponding antisense strand encoded by a sequence selected from SEQ ID NOs: 114-116, respectively. In some embodiments, the target RNA is a TNF-alpha mRNA, and the siRNA comprises a sense strand encoded by a sequence selected from SEQ ID NOs: 87-89. In some embodiments, the target RNA is a TNF-alpha mRNA, and the siRNA comprises a sense strand encoded by a sequence selected from SEQ ID NOs: 87-89, and a corresponding antisense strand encoded by a sequence selected from SEQ ID NOs: 117-119, respectively. In some embodiments, the target RNA is an IL-17 mRNA, and the siRNA comprises a sense strand encoded by a sequence selected from SEQ ID NOs: 90-92. In some embodiments, the target RNA is an IL-17 mRNA, and the siRNA comprises a sense strand encoded by a sequence selected from SEQ ID NOs: 90-92, and a corresponding antisense strand encoded by a sequence selected from SEQ ID NOs: 120-122, respectively. In some embodiments, the target RNA is an IL-6 mRNA, and the siRNA comprises a sense strand encoded by a sequence selected from SEQ ID NOs: 93-95. In some embodiments, the target RNA is an IL-6 mRNA, and the siRNA comprises a sense strand encoded by a sequence selected from SEQ ID NOs: 93-95, and a corresponding antisense strand encoded by a sequence selected from SEQ ID NOs: 123-125, respectively. In some embodiments, the target RNA is an IL-6R alpha mRNA, and the siRNA comprises a sense strand encoded by a sequence selected from SEQ ID NOs: 96 and 97. In some embodiments, the target RNA is an IL-6R alpha mRNA, and the siRNA comprises a sense strand encoded by a sequence selected from SEQ ID NOs: 96 and 97, and a corresponding antisense strand encoded by a sequence selected from SEQ ID NOs: 125 and 127, respectively. In some embodiments, the target RNA is an IL-6R beta mRNA, and the siRNA comprises a sense strand encoded by the sequence set forth in SEQ ID NO: 98. In some embodiments, the target RNA is an IL-6R beta mRNA, and the siRNA comprises a sense strand encoded by the sequence set forth in SEQ ID NO: 98, and a corresponding antisense strand encoded by the sequence set forth in SEQ ID NO: 128. In some embodiments, the target RNA is an ACE2 mRNA, and the siRNA comprises a sense strand encoded by a sequence selected from SEQ ID NOs: 99-101. In some embodiments, the target RNA is an ACE2 mRNA, and the siRNA comprises a sense strand encoded by the sequence set forth in selected from SEQ ID NOs: 99-101, and a corresponding antisense strand encoded by a sequence selected from SEQ ID NOs: 129-131, respectively. In some embodiments, the target RNA is a SARS CoV-2 ORF1ab mRNA, and the siRNA comprises a sense strand encoded by a sequence selected from SEQ ID NOs: 102-105. In some embodiments, the target RNA is a SARS CoV-2 ORF1ab mRNA, and the siRNA comprises a sense strand encoded by a sequence selected from SEQ ID NOs: 102-105, and a corresponding antisense strand encoded by a sequence selected from SEQ ID NOs: 132-135, respectively. In some embodiments, the target RNA is a SARS CoV-2 Spike Protein mRNA, and the siRNA comprises a sense strand encoded by a sequence selected from SEQ ID NOs: 106-108. In some embodiments, the target RNA is a SARS CoV-2 Spike Protein mRNA, and the siRNA comprises a sense strand encoded by a sequence selected from SEQ ID NOs: 106-108, and a corresponding antisense strand encoded by a sequence selected from SEQ ID NOs: 136-138, respectively. In some embodiments, the target RNA is a SARS CoV-2 Nucleocapsid Protein mRNA, and the siRNA comprises a sense strand encoded by the sequence set forth in SEQ ID NO: 109. In some embodiments, the target RNA is a SARS CoV-2 Nucleocapsid Protein mRNA, and the siRNA comprises a sense strand encoded by the sequence set forth in SEQ ID NO: 109, and a corresponding antisense strand encoded by the sequence set forth in SEQ ID NO: 139. In some aspects, the siRNA comprises a sense strand encoded by a sequence selected from SEQ ID NOs: 140-145. In some aspects, the siRNA comprises a sense strand encoded by a sequence selected from SEQ ID NOs: 140-145, and the corresponding antisense strand encoded by a sequence selected from SEQ ID NOs: 146-151. In some embodiments, the target RNA is an ALK2 mRNA, and the siRNA comprises a sense strand encoded by a sequence selected from SEQ ID NOs: 140-142. In some embodiments, the target RNA is an ALK2 mRNA, and the siRNA comprises a sense strand encoded by a sequence selected from SEQ ID NOs: 140-142, and a corresponding antisense strand encoded by a sequence selected from SEQ ID NOs: 146-148, respectively. In some embodiments, the target RNA is a SOD1 mRNA, and the siRNA comprises a sense strand encoded by a sequence selected from SEQ ID NOs: 143-145. In some embodiments, the target RNA is a SOD1 mRNA, and the siRNA comprises a sense strand encoded by a sequence selected from SEQ ID NOs: 143-145, and a corresponding antisense strand encoded by a sequence selected from SEQ ID NOs: 149-151, respectively.

### Gene of Interest

In some embodiments, the recombinant nucleic acid or RNA construct of the present invention may comprise two or more nucleic acid sequences encoding a gene of interest. In some embodiments, the recombinant nucleic acid or RNA construct of the present invention may comprise three nucleic acid sequences encoding a gene of interest. In some embodiments, the recombinant nucleic acid or RNA construct of the present invention may comprise four nucleic acid sequences encoding a gene of interest. In some embodiments, the recombinant nucleic acid or RNA construct of the present invention may comprise 5, 6, 7, 8, 9, 10, 11, 12, 13, 14, 15, or more nucleic acid sequences encoding a gene of interest. In one embodiment, each of the two or more nucleic acid sequences may encode a same gene of interest. In another embodiment, each of the two or more nucleic acid sequences encodes a different gene of interest. In some embodiments, each of the two or more nucleic acid sequences encoding the gene of interest comprises a nucleic acid sequence encoding a secretory protein. In some embodiments, each of the two or more nucleic acid sequences encoding the gene of interest may comprise a nucleic acid sequence encoding an intracellular protein. In some embodiments, each of the two or more nucleic acid sequences encoding the gene of interest comprises a nucleic acid sequence encoding an intraorganelle protein. In some embodiments, each of the two or more nucleic acid sequences encoding the gene of interest comprises a nucleic acid sequence encoding a membrane protein.

In some embodiments, the recombinant polynucleic acid or RNA construct may further comprise a nucleic acid sequence encoding or comprising a linker. In some embodiments, the nucleic acid sequence encoding or comprising the linker may connect each of the two or more nucleic acid sequences encoding the gene of interest. In some embodiments, the linker may be a non-cleavable linker. In a preferred embodiment, the linker may be a cleavable linker. In some embodiments, the linker may be a self-cleavable linker. Non-limiting examples of the linker comprise 2A peptide linker (or 2A self-cleaving peptides) such as T2A, P2A, E2A, or F2A, or tRNA linker, etc. In some embodiments, the linker is a T2A peptide linker. In some embodiments, the linker may be a P2A peptide linker. In some embodiments, the linker may be a E2A peptide linker. In some embodiments, the linker may be a F2A linker. In some embodiments, the linker may be a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising

(SEQ ID NO: 24)
AACAAAGCACCAGTGGTCTAGTGGTAGAATAGTACCCTGCCACGGTA
CAGACCCGGGTTCGATTCCCGGCTGGTGCA.

In some embodiments, the expression of the gene of interest is modulated by expressing an mRNA or a protein encoded by the gene of interest. In some embodiments, the expression of the gene of interest is upregulated by expressing an mRNA or a protein encoded by the gene of interest. Upregulation of the expression of an mRNA or a protein encoded by the gene of interest, as used herein, can refer to, but is not limited to, increasing the level of protein encoded by the gene of interest. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

In some embodiments, the gene of the interest encodes a protein. In some embodiments, the protein is a therapeutic protein. In a preferred embodiment of the present invention the protein is of human origin i.e., is a human protein. Non-limiting examples of proteins encoded by the gene of interest comprises: carboxypeptidases; cytokines; extracellular ligands and transporters; extracellular matrix proteins; glucosidases; glycosyltransferases; growth factors; growth factor binding proteins; heparin binding proteins; hormones; hydrolases; immunoglobulins; isomerases; kinases; lyases; metalloenzyme inhibitors; metalloproteases; milk proteins; neuroactive proteins; proteases; protease inhibitors; protein phosphatases; esterases; transferases; and vasoactive proteins all of human origin. In a more preferred embodiment of the present invention the protein of the present invention is a human protein selected from the group consisting of human carboxypeptidases; human cytokines; human extracellular ligands and transporters; human extracellular matrix proteins; human glucosidases; human glycosyltransferases; human growth factors; human growth factor binding proteins; human heparin binding proteins; human hormones; human hydrolases; human immunoglobulins; human isomerases; human kinases; human lyases; human metalloenzyme inhibitors; human metalloproteases; human milk proteins; human neuroactive proteins; human proteases; human protease inhibitors; human protein phosphatases; human esterases; human transferases; or human vasoactive proteins.

In one embodiment, the protein is selected from the group consisting of carboxypeptidases, wherein the carboxypeptidases are selected from the group consisting of ACE, ACE2, CNDP1, CPA1, CPA2, CPA4, CPA5, CPA6, CPB1, CPB2, CPE, CPN1, CPQ, CPXM1, CPZ, and SCPEP1; cytokines wherein the cytokines are selected from the group consisting of BMP1, BMP10, BMP15, BMP2, BMP3, BMP4, BMP5, BMP6, BMP7, BMP8A, BMP8B, C1QTNF4, CCL1, CCL11, CCL13, CCL14, CCL15, CCL16, CCL17, CCL18, CCL19, CCL2, CCL21, CCL22, CCL23, CCL24, CCL25, CCL26, CCL27, CCL28, CCL3, CCL3L1, CCL3L3, CCL4, CCL4L, CCL4L2, CCL5, CCL7, CCL8, CD40LG, CER1, CKLF, CLCF1, CNTF, CSF1, CSF2, CSF3, CTF1, CX3CL1, CXCL1, CXCL10, CXCL11, CXCL12, CXCL13, CXCL14, CXCL16, CXCL17, CXCL2, CXCL3, CXCL5, CXCL8, CXCL9, DKK1, DKK2, DKK3, DKK4, EDA, EBI3, FAM3B, FAM3C, FASLG, FLT3LG, GDF1, GDF10, GDF11, GDF15, GDF2, GDF3, GDF5, GDF6, GDF7, GDF9, GPI, GREM1, GREM2, GRN, IFNA1, IFNA13, IFNA10, IFNA14, IFNA16, IFNA17, IFNA2, IFNA21, IFNA4, IFNA5, IFNA6, IFNA7, IFNA8, IFNB1, IFNE, IFNG, IFNK, IFNL1, IFNL2, IFNL3, IFNL4, IFNW1, IL10, IL11, IL12A, IL12B, IL13, IL15, IL16, IL17A, IL17B, IL17C, IL17D, IL17F, IL18, IL19, IL1A, IL1B, IL1F10, IL2, IL20, IL21, IL22, IL23A, IL24, IL25, IL26, IL27, IL3, IL31, IL32, IL33, IL34, IL36A, IL36B, IL36G, IL36RN, IL37, IL4, IL5, IL6, IL7, IL9, LEFTY1, LEFTY2, LIF, LTA, MIF, MSTN, NAMPT, NODAL, OSM, PF4, PF4V1, SCGB3A1, SECTM1, SLURP1, SPP1, THNSL2, THPO, TNF, TNFSF10, TNFSF11, TNFSF12, TNFSF13, TNFSF13B, TNFSF14, TNFSF15, TSLP, VSTM1, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, XCL1, and XCL2; extracellular ligands and transporters, wherein the extracellular ligands and transporters are selected from the group consisting of APCS, CHI3L1, CHI3L2, CLEC3B, DMBT1, DMKN, EDDM3A, EDDM3B, EFNA4, EMC10, ENAM, EPYC, ERVH48-1, F13B, FCN1, FCN2, GLDN, GPLD1, HEG1, ITFG1, KAZALD1, KCP, LACRT, LEG1, METRN, NOTCH2NL, NPNT, OLFM1, OLFML3, PRB2, PSAP, PSAPL1, PSG1, PSG6, PSG9, PTX3, PTX4, RBP4, RNASE10, RNASE12, RNASE13, RNASE9, RSPRY1, RTBDN, S100A12, S100A13, S100A7, S100A8, SAA2, SAA4, SCG1, SCG2, SCG3, SCGB1C1, SCGB1C2, SCGB1D1, SCGB1D2, SCGB1D4, SCGB2B2, SCGB3A2, SCGN, SCRG1, SCUBE1, SCUBE2, SCUBE3, SDCBP, SELENOP, SFTA2, SFTA3, SFTPA1, SFTPA2, SFTPC, SFTPD, SHBG, SLURP2, SMOC1, SMOC2, SMR3A, SMR3B, SNCA, SPATA20, SPATA6, SOGA1, SPARC, SPARCL1, SPATA20, SPATA6, SRPX2, SSC4D, STX1A, SUSD4, SVBP, TCN1, TCN2, TCTN1, TF, TULP3, TFF2, TFF3, THSD7A, TINAG, TINAGL1, TMEFF2, TMEM25, and VWC2L; extracellular matrix proteins, wherein the extracellular matrix proteins are selected from the group consisting of ABI3BP, AGRN, CCBE1, CHL1, COL15A1, COL19A1, COLEC11, DMBT1, DRAXIN, EDIL3, ELN, EMID1, EMILIN1, EMILIN2, EMILIN3, EPDR1, FBLN1, FBLN2, FBLN5, FLRT1, FLRT2, FLRT3, FREM1, GLDN, IBSP, KERA, KIAA0100, KIRREL3, KRT10, LAMB2, MGP, RPTN, SBSPON, SDC1, SDC4, SEMA3A, SEMA3B, SEMA3C, SEMA3D, SEMA3E, SEMA3F, SEMA3G, SIGLEC1, SIGLEC10, SIGLEC6, SLIT1, SLIT2, SLIT3, SLITRK1, SNED1, SNORC, SPACA3, SPACA7, SPON1, SPON2, STATH, SVEP1, TECTA, TECTB, TNC, TNN, TNR and TNXB; glucosidases, wherein the glucosidases are selected from the group consisting of AMY1A, AMY1B, AMY1C, AMY2A, AMY2B, CEMIP, CHIA, CHIT1, FUCA2, GLB1L, GLB1L2, HPSE, HYAL1, HYAL3, KL, LYG1, LYG2, LYZL1, LYZL2, MAN2B2, SMPD1, SMPDL3B, SPACA5, and SPACA5B; glycosyltransferases, wherein the glycosyltransferases are selected from the group consisting of ARTS, B4GALT1, EXTL2, GALNT1, GALNT2, GLT1D1, MGAT4A, ST3GAL1, ST3GAL2, ST3GAL3, ST3GAL4, ST6GAL1, and XYLT1; growth factors, wherein the growth factors are selected from the group consisting of AMH, ARTN, BTC, CDNF, CFC1, CFC1B, CHRDL1, CHRDL2, CLEC11A, CNMD, EFEMP1, EGF, EGFL6, EGFL7, EGFL8, EPGN, EREG, EYS, FGF1, FGF10, FGF16, FGF17, FGF18, FGF19, FGF2, FGF20, FGF21, FGF22, FGF23, FGF3, FGF4, FGF5, FGF6, FGF7, FGF8, FGF9, FRZB, GDNF, GFER, GKN1, HBEGF, HGF, IGF-1, IGF2, INHA, INHBA, INHBB, INHBC, INHBE, INS, KITLG, MANF, MDK, MIA, NGF, NOV, NRG1, NRG2, NRG3, NRG4, NRTN, NTF3, NTF4, OGN, PDGFA, PDGFB, PDGFC, PDGFD, PGF, PROK1, PSPN, PTN, SDF1, SDF2, SFRP1, SFRP2, SFRP3, SFRP4, SFRP5, TDGF1, TFF1, TGFA, TGFB1, TGFB2, TGFB3, THBS4, TIMP1, VEGFA, VEGFB, VEGFC, VEGFD, and WISP3; growth factor binding proteins, wherein the growth factor binding proteins are selected from the group consisting of CHRD, CYR61, ESM1, FGFBP1, FGFBP2, FGFBP3, HTRA1, GHBP, IGFALS, IGFBP1, IGFBP2, IGFBP3, IGFBP4, IGFBP5, IGFBP6, IGFBP7, LTBP1, LTBP2, LTBP3, LTBP4, SOSTDC1, NOG, TWSG1, and WIF1; heparin binding proteins, wherein the heparin binding proteins are selected from the group consisting of ADA2, ADAMTSL5, ANGPTL3, APOB, APOE, APOH, COL5A1, COMP, CTGF, FBLN7, FN1, FSTL1, HRG, LAMC2, LIPC, LIPG, LIPH, LIPI, LPL, PCOLCE2, POSTN, RSPO1, RSPO2, RSPO3, RSPO4, SAA1, SLIT2, SOST, THBS1, and VTN; hormones, wherein the hormones are selected from the group consisting of ADCYAP1, ADIPOQ, ADM, ADM2, ANGPTL8, APELA, APLN, AVP, C1QTNF12, C1QTNF9, CALCA, CALCB, CCK, CGA, CGB1, CGB2, CGB3, CGB5, CGB8, COPA, CORT, CRH, CSH1, CSH2, CSHL1, ENHO, EPO, ERFE, FBN1, FNDC5, FSHB, GAL, GAST, GCG, GH, GH1, GH2, GHRH, GHRL, GIP, GNRH1, GNRH2, GPHA2, GPHB5, IAPP, INS, INSL3, INSL4, INSL5, INSL6, LHB, METRNL, MLN, NPPA, NPPB, NPPC, OSTN, OXT, PMCH, PPY, PRL, PRLH, PTH, PTHLH, PYY, RETN, RETNLB, RLN1, RLN2, RLN3, SCT, SPX, SST, STC1, STC2, TG, TOR2A, TRH, TSHB, TTR, UCN, UCN2, UCN3, UTS2, UTS2B, and VIP; hydrolases, wherein the hydrolases are selected from the group consisting of AADACL2, ABHD15, ACP7, ACPP, ADA2, ADAMTSL1, AOAH, ARSF, ARSI, ARSJ, ARSK, BTD, CHI3L2, ENPP1, ENPP2, ENPP3, ENPP5, ENTPD5, ENTPD6, GBP1, GGH, GPLD1, HPSE, LIPC, LIPF, LIPG, LIPH, LIPI, LIPK, LIPM, LIPN, LPL, PGLYRP2, PLA1A, PLA2G10, PLA2G12A, PLA2G1B, PLA2G2A, PLA2G2D, PLA2G2E, PLA2G2F, PLA2G3, PLA2G5, PLA2G7, PNLIP, PNLIPRP2, PNLIPRP3, PON1, PON3, PPT1, SMPDL3A, THEM6, THSD1, and THSD4; immunoglobulins, wherein the immunoglobulins are selected from the group consisting of IGSF10, IGKV1-12, IGKV1-16, IGKV1-33, IGKV1-6, IGKV1D-12, IGKV1D-39, IGKV1D-8, IGKV2-30, IGKV2D-30, IGKV3-11, IGKV3D-20, IGKV5-2, IGLC1, IGLC2, and IGLC3; isomerases, wherein the isomerases are selected from the group consisting of NAXE, PPIA, and PTGDS; kinases, wherein the kinases are selected from the group consisting of ADCK1, ADPGK, FAM20C, ICOS, and PKDCC; lyases, wherein the lyases are selected from the group consisting of PM20D1, PAM, and CA6; metalloenzyme inhibitors, wherein the metalloenzyme inhibitors are selected from the group consisting of FETUB, SPOCK3, TIMP2, TIMP3, TIMP4, WFIKKN1, and WFIKKN2; metalloproteases, wherein the metalloproteases are selected from the group consisting of ADAM12, ADAM28, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, CLCA1, CLCA2, CLCA4, IDE, MEP1B, MMEL1, MMP1, MMP10, MMP11, MMP12, MMP13, MMP16, MMP17, MMP19, MMP2, MMP20, MMP21, MMP24, MMP25, MMP26, MMP28, MMP3, MMP1, MMP8, MMP9, PAPPA, PAPPA2, TLL1, and TLL2; milk proteins, wherein the milk proteins are selected from the group consisting of CSN1S1, CSN2, CSN3, and LALBA; neuroactive proteins, wherein the neuroactive proteins are selected from the group consisting of CARTPT, NMS, NMU, NPB, NPFF, NPS, NPVF, NPW, NPY, PCSK1N, PDYN, PENK, PNOC, POMC, PROK2, PTH2, PYY2, PYY3, QRFP, TAC1, and TAC3; proteases, wherein the proteases are selected from the group consisting of ADAMTS6, C1R, C1RL, C2, CASP4, CELA1, CELA2A, CELA2B, CFB, CFD, CFI, CMA1, CORIN, CTRB1, CTRB2, CTSB, CTSD, DHH, F10, F11, F12, F2, F3, F7, F8, F9, FAP, FURIN, GZMA, GZMK, GZMM, HABP2, HGFAC, HTRA3, HTRA4, IHH, KLK10, KLK11, KLK12, KLK13, KLK14, KLK15, KLK3, KLK4, KLK5, KLK6, KLK7, KLK8, KLK9, KLKB1, MASP1, MASP2, MST1L, NAPSA, OVCH1, OVCH2, PCSK2, PCSK5, PCSK6, PCSK9, PGA3, PGA4, PGA5, PGC, PLAT, PLAU, PLG, PROC, PRSS1, PRSS12, PRSS2, PRSS22, PRSS23, PRSS27, PRSS29P, PRSS3, PRSS33, PRSS36, PRSS38, PRSS3P2, PRSS42, PRSS44, PRSS47, PRSS48, PRSS53, PRSS57, PRSS58, PRSS8, PRTN3, RELN, REN, TMPRSS11D, TMPRSS11E, TMPRSS2, TPSAB1, TPSB2, and TPSD1; protease inhibitors, wherein the protease inhibitors are selected from the group consisting of A2M, A2ML1, AMBP, ANOS1, COL28A1, COL6A3, COL7A1, CPAMD8, CST1, CST2, CST3, CST4, CST5, CST6, CST7, CST8, CST9, CST9L, CST9LP1, CSTL1, EPPIN, GPC3, HMSD, ITIH1, ITIH2, ITIH3, ITIH4, ITIH5, ITIH6, KNG1, OPRPN, OVOS1, OVOS2, PAPLN, PI15, PI16, PI3, PZP, R3HDML, SERPINA1, SERPINA10, SERPINA11, SERPINA12, SERPINA13P, SERPINA3, SERPINA4, SERPINA5, SERPINA7, SERPINA9, SERPINB2, SERPINB5, SERPINC1, SERPINE1, SERPINE2, SERPINE3, SERPINF2, SERPING1, SERPINI1, SERPINI2, SPINK1, SPINK13, SPINK14, SPINK2, SPINK4, SPINK5, SPINK6, SPINK7, SPINK8, SPINK9, SPINT1, SPINT3, SPINT4, SPOCK1, SPOCK2, SPP2, SSPO, TFPI, TFPI2, WFDC1, WFDC10A, WFDC13, WFDC2, WFDC3, WFDC5, WFDC6, and WFDC8; protein phosphatases, wherein the protein phosphatases are selected from the group consisting of ACP7, ACPP, PTEN, and PTPRZ1; esterases, wherein the esterases, are selected from the group consisting of BCHE, CEL, CES4A, CES5A, NOTUM, and SIAE; transferases, wherein the transferases, are selected from the group consisting of METTL24, FKRP, CHSY1, CHST9, and B3GAT1; and vasoactive proteins, wherein the vasoactive proteins are selected from the group consisting of AGGF1, AGT, ANGPT1, ANGPT2, ANGPTL4, ANGPTL6, EDN1, EDN2, EDN3, and NTS. In some embodiments, the protein is selected from the group consisting of Insulin-like Growth Factor 1 (IGF-1), Interleukin 4 (IL-4), Interferon beta (IFN beta), Interferon alpha (IFN alpha), ACE2 soluble receptor, Interleukin 37 (IL-37), and Interleukin 38 (IL-38). In some embodiments, the protein is selected from the group consisting of Insulin-like Growth Factor 1 (IGF-1), Interleukin 4 (IL-4), Interferon beta (IFN beta), and ACE2 soluble receptor. In some embodiments, the protein is selected from the group consisting of Insulin-like Growth Factor 1 (IGF-1), Interleukin 4 (IL-4), Interferon beta (IFN beta), ACE2 soluble receptor, and Erythropoietin (EPO). In some embodiments, the protein is selected from the group consisting of Insulin-like Growth Factor 1 (IGF-1), and Interleukin 4 (IL-4). In some embodiments, the protein is IGF-1. In some embodiments, the protein is IL-4. In some embodiments, the protein is Interferon beta (IFN beta). In some embodiments, the protein is ACE2 soluble receptor. In some embodiments, the protein is Erythropoietin (EPO).

In one embodiment of the present invention, the recombinant polynucleic acid or RNA construct comprising a nucleic acid sequence or an mRNA encoding a gene of interest may comprise a nucleic acid sequence encoding human insulin-like growth factor 1 (IGF-1). In another embodiment, the recombinant polynucleic acid or RNA construct can be naked DNA or RNA comprising a nucleic acid sequence encoding IGF-1. In this embodiment of the present invention, the recombinant polynucleic acid or RNA construct may comprise a nucleic acid sequence encoding the mature human IGF-1. In a preferred embodiment of the present invention, the recombinant polynucleic acid or RNA construct may comprise a nucleic acid sequence encoding a propeptide of IGF-1, preferably a propeptide of human IGF-1, and a nucleic acid sequence encoding a mature protein of IGF-1, or preferably a mature protein of human IGF-1, and does not comprise a nucleic acid sequence encoding an E-peptide of IGF-1, preferably does not comprise a nucleic acid sequence encoding a human E-peptide of IGF-1, i.e., IGF-1 with a carboxyl-terminal extension. In a more preferred embodiment of the present invention, the recombinant polynucleic acid or RNA construct may comprise a nucleic acid sequence encoding a propeptide of IGF-1, preferably a propeptide of human IGF-1, a nucleic acid sequence encoding a mature protein of IGF-1, or preferably a mature protein of human IGF-1. Preferably the recombinant polynucleic acid or RNA construct does not comprise a nucleic acid sequence encoding an E-peptide of IGF-1, or more preferably does not comprise a nucleic acid sequence encoding a human E-peptide of IGF-1. In a further preferred embodiment of the present invention, the recombinant polynucleic acid or RNA construct may comprise a nucleic acid sequence encoding a propeptide of IGF-1, preferably a propeptide of human IGF-1, a nucleic acid sequence encoding a mature protein of IGF-1, or preferably a mature protein of human IGF-1 and a nucleic acid sequence encoding the signal peptide of the brain-derived neurotrophic factor (BDNF). Preferably the recombinant polynucleic acid or RNA construct does not comprise a nucleic acid sequence encoding an E-peptide of IGF-1, and more preferably does not comprise a nucleic acid sequence encoding a human E-peptide of IGF-1.

In some embodiments, the recombinant polynucleic acid or RNA construct may comprise a nucleic acid sequence encoding a propeptide (also called pro-domain) of IGF-1, preferably of human IGF-1 having 27 amino acids, and a nucleic sequence encoding a mature IGF-1, preferably a mature human IGF-1 having 70 amino acids, and preferably does not comprise a nucleotide sequence encoding an E-peptide of IGF-1, and preferably does not comprise a nucleic acid sequence encoding a human E-peptide of IGF-1. In some embodiments, the recombinant polynucleic acid or RNA construct may comprise a nucleic acid sequence encoding a propeptide (also called pro-domain) of IGF-1, preferably of human IGF-1 having 27 amino acids, a nucleic sequence encoding a mature IGF-1, preferably a mature human IGF-1 having 70 amino acids and a nucleic acid sequence encoding the signal peptide of the brain-derived neurotrophic factor (BDNF). Preferably the recombinant polynucleic acid or RNA construct does not comprise a nucleic sequence encoding an E-peptide of IGF-1, more preferably does not comprise a nucleic acid sequence encoding a human E-peptide of IGF-1.

In some embodiments, the recombinant polynucleic acid or RNA construct of the present invention may comprise a nucleic acid sequence encoding a propeptide (also called pro-domain) of human IGF-1 having 27 amino acids, and a nucleic acid sequence encoding a mature human IGF-1 having 70 amino acids and preferably does not comprise a nucleic acid sequence encoding an E-peptide (also called E-domain) of human IGF-1, wherein the nucleic acid sequence encoding the propeptide (also called pro-domain) of human IGF-1 having 27 amino acids, and the nucleic acid sequence encoding the mature human IGF-1 having 70 amino acids and the nucleic acid sequence encoding the E-peptides are as referred to in the Uniprot database as UniProtKB—P05019 and in the Genbank database as NM_000618.4, NM_001111285.2 and NM_001111283.2, respectively.

In some embodiments, the gene of interest (which can encode, e.g., an mRNA of interest and/or a protein of interest corresponding to the gene of interest), encodes a protein of interest, wherein the protein of interest is an anti-inflammatory cytokine. In some embodiments, the anti-inflammatory cytokine is an interferon or an interleukin. In some embodiments, the interferon is a Type I interferon (e.g., IFN-α, IFN-δ, IFN-ε, IFN-κ, IFN-ν, IFN-τ, and IFN-ω), a Type II interferon (IFN-γ), or a Type III interferon (IFN-λ). In some embodiments, an alpha interferon is selected from interferon alpha-n3, interferon alpha-2a, and interferon alpha-2b. The activities of interferons against viral infections have been described, e.g., in WO 2004/096852 (Chen, et al.) describing an anti-SARS effect of IFN-ω, and WO 2005/097165 (Klucher, et al.), describing an anti-viral effect of IFN-λ, variants, both incorporated herein by reference. In some embodiments, the cytokine is an interleukin. In some embodiments, the interleukin is an interleukin 1F family member. In some embodiments, the interleukin is interleukin 37 (IL-37, formerly known as the interleukin-1 family member 7 or IL-1F7, and described by, e.g., Yan, et al., 2018, Mediators of Inflammation Volume 2019, Article ID 2650590, and Conti, et al., March-April 2020, Journal of biological regulators and homeostatic agents 34(2), doi: 10.23812/CONTI-E [Epub ahead of print], both incorporated herein by reference). In some embodiments, the interleukin is interleukin 38 (formerly known as IL-1HY2, and described by, e.g., Xu, et al., June 2018, Frontiers in Immunology vol. 9, article. 1462, incorporated herein by reference). In some embodiments, the gene of interest encodes a decoy protein. In some embodiments the decoy protein is a soluble form of the virus host cell receptor. In some embodiments, the decoy protein is soluble ACE2 receptor. In some embodiments, the gene of interest encodes a protein selected from: a Type I interferon, a Type II interferon, a Type III interferon, an interleukin, and a decoy protein. In some embodiments, the gene of interest encodes a protein selected from: an IFN-α, e.g., interferon alpha-n3, interferon alpha-2a, or interferon alpha-2b, an IFN-β, an IFN-δ, an IFN-ε, an IFN-κ, an IFN-ν, an IFN-τ, an IFN-ω, an IFN-γ, an IFN-λ, IL-37, IL-38, and soluble ACE2 receptor.

### Target Motif

In some embodiments, the compositions described herein comprise a recombinant polynucleic acid or an RNA construct comprising a target motif. The term “target motif” or “targeting motif” as used herein can refer to any short peptide present in the newly synthesized polypeptides or proteins that are destined to any parts of cell membranes, extracellular compartments, or intracellular compartments. Intracellular compartments include, but are not limited to, intracellular organelles such as nucleus, nucleolus, endosome, proteasome, ribosome, chromatin, nuclear envelope, nuclear pore, exosome, melanosome, Golgi apparatus, peroxisome, endoplasmic reticulum (ER), lysosome, centrosome, microtubule, mitochondria, chloroplast, microfilament, intermediate filament, or plasma membrane. Other terms include, but are not limited to, signal sequence, targeting signal, localization signal, localization sequence, transit peptide, leader sequence, or leader peptide. In some embodiments, the target motif is operably linked to the at least one nucleic acid sequence encoding the gene of interest. Non-limiting examples of the target motif comprise a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, a centrosomal localization signal (CLS) or any other signal that targets a protein to a certain part of cell membrane, extracellular compartments, or intracellular compartments.

In some embodiments, the target motif is selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest, wherein the target motif homologous to the protein encoded by the gene of interest is modified by insertion, deletion, and/or substitution of at least one amino acid; and (d) a naturally occurring amino acid sequence which does not have the function of a target motif in nature, wherein the naturally occurring amino acid sequence is optionally modified by insertion, deletion, and/or substitution of at least one amino acid.

In some embodiments, the target motif is a signal peptide. In some embodiments, the signal peptide is selected from the group consisting of: (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide homologous to a protein encoded by the gene of interest, wherein the signal peptide homologous to the protein encoded by the gene of interest is modified by insertion, deletion, and/or substitution of at least one amino acid; and (d) a naturally occurring amino acid sequence which does not have the function of a signal peptide in nature, wherein the naturally occurring amino acid sequence is optionally modified by insertion, deletion, and/or substitution of at least one amino acid. In some embodiments, the amino acids 1-9 of the N-terminal end of the signal peptide have an average hydrophobic score of above 2.

The term “target motif heterologous to a protein encoded by the gene of interest” or “signal peptide heterologous to a protein encoded by the gene of interest” as used herein refers to a naturally occurring target motif or signal peptide which is different to the naturally occurring target motif or signal peptide of the protein, i.e., the target motif or the signal peptide is not derived from the same gene of the protein. Usually a target motif or a signal peptide heterologous to a given protein is a target motif or a signal peptide from another protein, which is not related to the given protein i.e., which has an amino acid sequence which differs from the target motif or the signal peptide of the given protein, e.g., which has an amino acid sequence which differs from the target motif or the signal peptide of the given protein by more than 50%, preferably by more than 60%, more preferably by more than 70%, even more preferably by more than 80%, most preferably by more than 90%, or in particular by more than 95%. Preferably a target motif or a signal peptide heterologous to a given protein has a sequence identity with the amino acid sequence of the naturally occurring (homologous) target motif or signal peptide of the given protein of less than 95%, preferably less than 90%, more preferably less than 80%, even more preferably less than 70%, most preferably less than 60%, or in particular, less than 50%. Although heterologous sequences may be derived from the same organism, they naturally (in nature) do not occur in the same nucleic acid molecule, such as in the same mRNA. The target motif or the signal peptide heterologous to a protein and the protein to which the target motif or the signal peptide is heterologous can be of the same or different origin and are usually of the same origin, preferably of eukaryotic origin, more preferably of eukaryotic origin of the same eukaryotic organism, even more preferably of mammalian origin, in particular of mammalian origin of the same mammalian organism, or more particular of human origin. For example, a recombinant polynucleic acid or RNA construct comprising a nucleic acid sequence encoding the human BDNF signal peptide and the human IGF-1 gene, i.e., a signal peptide heterologous to a protein wherein the signal peptide and the protein are of the same origin, namely of human origin is disclosed.

The term “target motif homologous to a protein encoded by the gene of interest” or “signal peptide homologous to a protein encoded by the gene of interest” as used herein refers to the naturally occurring target motif or signal peptide of a protein. A target motif or a signal peptide homologous to a protein is the target motif or the signal peptide encoded by the gene of the protein as it occurs in nature. A target motif or a signal peptide homologous to a protein is usually of eukaryotic origin e.g., the naturally occurring target motif or signal peptide of a eukaryotic protein, preferably of mammalian origin e.g., the naturally occurring target motif or signal peptide of a mammalian protein, or more preferably of human origin e.g., the naturally occurring target motif or signal peptide of a human protein.

The term “naturally occurring amino acid sequence which does not have the function of a target motif in nature” or “naturally occurring amino acid sequence which does not have the function of a signal peptide in nature” as used herein refers to an amino acid sequence which occurs in nature and which is not identical to the amino acid sequence of any target motif or signal peptide occurring in nature. The naturally occurring amino acid sequence which does not have the function of a target motif or a signal peptide in nature as referred to in the present invention is preferably between 10-50, more preferably 11-45, even more preferably 12-45, most preferably 13-45, in particular 14-45, more particular 15-45, or even more particular 16-40 amino acids long. Preferably the naturally occurring amino acid sequence which does not have the function of a target motif or a signal peptide in nature of the present invention is of eukaryotic origin and not identical to any target motif or signal peptide of eukaryotic origin, more preferably is of mammalian origin and not identical to any target motif or signal peptide of mammalian origin, or more preferably is of human origin and not identical to any target motif or signal peptide of human origin occurring in nature. A naturally occurring amino acid sequence which does not have the function of a target motif or a signal peptide in nature is usually an amino acid sequence of the coding sequence of a protein. A naturally occurring amino acid sequence which does not have the function of a target motif or a signal peptide in nature according to the present invention is usually of eukaryotic origin, preferably of mammalian origin, or more preferably of human origin. The term “naturally occurring,” “natural,” and “in nature” as used herein have the equivalent meaning.

The term “amino acids 1-9 of the N-terminal end of the signal peptide” as used herein refers to the first nine amino acids of the N-terminal end of the amino acid sequence of a signal peptide. Analogously the term “amino acids 1-7 of the N-terminal end of the signal peptide” as used herein refers to the first seven amino acids of the N-terminal end of the amino acid sequence of a signal peptide and the term “amino acids 1-5 of the N-terminal end of the signal peptide” as used herein refers to the first five amino acids of the N-terminal end of the amino acid sequence of a signal peptide.

The term “amino acid sequence modified by insertion, deletion, and/or substitution of at least one amino acid” as used herein refers to an amino acid sequence which includes an amino acid substitution, insertion, and/or deletion of at least one amino acid within the amino acid sequence. The term “target motif heterologous to a protein encoded by the gene of interest is modified by insertion, deletion, and/or substitution of at least one amino acid” or “signal peptide heterologous to a protein encoded by the gene of interest is modified by insertion, deletion, and/or substitution of at least one amino acid” as used herein refers to an amino acid sequence of a naturally occurring target motif or signal peptide heterologous to a protein which includes an amino acid substitution, insertion, and/or deletion of at least one amino acid within its naturally occurring amino acid sequence. The term “target motif homologous to a protein encoded by the gene of interest is modified by insertion, deletion, and/or substitution of at least one amino acid” or “signal peptide homologous to a protein encoded by the gene of interest is modified by insertion, deletion, and/or substitution of at least one amino acid” as used herein refers to a naturally occurring target motif or signal peptide homologous to a protein which includes an amino acid substitution, insertion, and/or deletion of at least one amino acid within its naturally occurring amino acid sequence. The term “the naturally occurring amino acid sequence is modified by insertion, deletion, and/or substitution of at least one amino acid” refers to a naturally occurring amino acid sequence which includes an amino acid substitution, insertion, and/or deletion of at least one amino acid within its naturally occurring amino acid sequence. By “amino acid substitution” or “substitution” herein may refer to the replacement of an amino acid at a particular position in a parent protein sequence with another amino acid. For example, the substitution R34K refers to a polypeptide, in which the arginine at position 34 is replaced with a lysine. For the preceding example, 34K indicates the substitution of an amino acid at position 34 with a lysine. For the purposes herein, multiple substitutions are typically separated by a slash. For example, R34K/L78V refers to a double variant comprising the substitutions R34K and L38V. By “amino acid insertion” or “insertion” as used herein may refer to the addition of an amino acid at a particular position in a parent protein sequence. For example, insert −34 designates an insertion at position 34. By “amino acid deletion” or “deletion” as used herein may refer to the removal of an amino acid at a particular position in a parent protein sequence. For example, R34-designates the deletion of arginine at position 34.

Preferably the deleted amino acid is an amino acid with a hydrophobic score of below −0.8, preferably below 1.9. Preferably the substitute amino acid is an amino acid with a hydrophobic score which is higher than the hydrophobic score of the substituted amino acid, more preferably the substitute amino acid is an amino acid with a hydrophobic score of 2.8 and higher, or more preferably with a hydrophobic score of 3.8 and higher. Preferably the inserted amino acid is an amino acid with a hydrophobic score of 2.8 and higher, or more preferably with a hydrophobic score of 3.8 and higher.

Usually between 1 and 15, preferably between 1 and 11 amino acids, more preferably between 1 and 10 amino acids, even more preferably between 1 and 9 amino acids, in particular between 1 and 8 amino acids, more particular between 1 and 7 amino acids, even more particular between 1 and 6 amino acids, particular preferably between 1 and 5 amino acids, more particular preferably between 1 and 4 amino acids, or even more particular preferably between 1 and 2 amino acids in a given amino acid sequence are inserted, deleted, and/or substituted. Usually between 1 and 15, preferably between 1 and 11 amino acids, more preferably between 1 and 10 amino acids, even more preferably between 1 and 9 amino acids, in particular between 1 and 8 amino acids, more particular between 1 and 7 amino acids, even more particular between 1 and 6 amino acids, particular preferably between 1 and 5 amino acids, more particular preferably between 1 and 4 amino acids, or even more particular preferably between 1 and 2 amino acids in a given amino acid sequence are inserted, deleted, and/or substituted usually within the amino acids 1-11, preferably within the amino acids 1-10, more preferably within the amino acids 1-9, even more preferably within the amino acids 1-8, in particular within the amino acids 1-7, more particular within the amino acids 1-6, even more particular within the amino acids 1-5, particular preferably within the amino acids 1-4, more particular preferably within the amino acids 1-3, or even more particular preferably within the amino acids 1-2 of the N-terminal end of the amino acid sequence of the target motif or the signal peptide. Preferably the amino acid sequence is optionally modified by deletion, and/or substitution of at least one amino acid.

Preferably, the average hydrophobic score of the first nine amino acids of the N-terminal end of the amino acid sequence of the modified signal peptide is increased 1.0 unit or above compared to the signal peptide without modification.

The term “insulin-like growth factor 1,” “insulin-like growth factor 1 (IGF1 or IGF-1),” “IGF1,” or “IGF-1” as used herein usually refers to the natural sequence of the IGF-1 protein without the signal peptide and may comprise the propeptide and/or the E-peptide and preferably refers to the natural sequence of the IGF-1 protein without the signal peptide and without the E-peptide. The term “human insulin-like growth factor 1 (IGF-1)” as used herein refers to the natural sequence of human IGF-1 (pro-IGF-1 which is referred to in the Uniprot database as UniProtKB—P05019 and in the Genbank database as NM_000618.4, NM_001111285.2 and NM_001111283.2, or a fragment thereof. The natural DNA sequence encoding human insulin-like growth factor 1 may be codon-optimized. The natural sequence of human IGF-1 consists of the human signal peptide having 21 amino acids (nucleotides 1-63), the human propeptide (also called pro-domain) having 27 amino acids (nucleotides 64-144), the mature human IGF-1 having 70 amino acids (nucleotides 145-354) and the C-terminal domain of human IGF-1 which is the so-called E-peptide (or E-domain). The C-terminal domain of human IGF-1 (so called E-peptide or E-domain) comprises the Ea-, Eb-, or Ec-domain which are generated by alternative splicing events. The Ea-domain consists or 35 amino acids (105 nucleotides), the Eb-domain consists of 77 amino acids (231 nucleotides), and the Ec-domain consists of 40 amino acids (120 nucleotides) (see e.g., Wallis M (2009) New insulin-like growth factor (IGF)-precursor sequences from mammalian genomes: the molecular evolution of IGFs and associated peptides in primates. Growth Horm IGF Res 19(1):12-23. doi: 10.1016/j.ghir.2008.05.001). The term “human insulin-like growth factor 1 (IGF-1)” as used herein usually refers to the natural sequence of the human IGF-1 protein without the signal peptide and may comprise the propeptide and/or the E-peptide and preferably refers to the natural sequence of the human IGF-1 protein without the signal peptide and without the E-peptide. The term “human insulin-like growth factor 1 (IGF-1)” as used herein usually comprises the mature human IGF-1. The term “mature protein” refers to the protein synthesized in the endoplasmic reticulum and secreted via the Golgi apparatus in a cell expressing and

secreting the protein. The term “mature IGF-1” refers to the protein synthesized in the endoplasmic reticulum and secreted via the Golgi apparatus in a cell expressing and secreting IGF-1. The term “mature human IGF-1” refers to the protein synthesized in the endoplasmic reticulum and secreted via the Golgi apparatus in a human cell expressing and secreting human IGF-1 and normally contains the amino acids encoded by nucleotide as shown in SEQ ID NO: 19.

SEQ ID NO: 19
GGACCTGAGACACTTTGTGGCGCTGAACTGGTGGACGCCCTGCAGTT
TGTGTGTGGCGACAGAGGCTTCTACTTCAACAAGCCCACAGGCTACG
GCAGCAGCTCTAGAAGGGCTCCTCAGACCGGAATCGTGGACGAGTGC
TGTTTCAGAAGCTGCGACCTGCGGCGGCTGGAAATGTATTGTGCCCC
TCTGAAGCCTGCCAAGAGCGCC

The term “signal peptide of the Insulin growth factor 1 (IGF-1) Modified,” “modified signal peptide of IGF-1,” or “signal peptide of IGF-1-Modified” as used herein refers to the modified signal peptide of IGF-1 wherein natural signal peptide of IGF-1 which is referred to in the Uniprot database as P05019 and in the Genbank database as NM_000618.4, NM_001111284.1 and NM_001111285.2 is modified by the substitutions G2L/S5L/T9L/Q10L and deletions K3- and C15- and has preferably the amino acid sequence as shown in SEQ ID NO: 20 and/or is preferably encoded by the DNA sequence as shown in SEQ ID NO: 21.

SEQ ID NO: 20
Met-Leu-Ile-Leu-Leu-Leu-Pro-Leu-Leu-Leu-Phe-
Lys-Cys-Phe-Cys-Asp-Phe-Leu-Lys
SEQ ID NO: 21
ATGCTGATTCTGCTGCTGCCCCTGCTGCTGTTCAAGTGCTTCTGCGA
CTTCCTGAAA

The term “Insulin growth factor 1 (IGF-1) pro domain modified,” “modified IGF-1 pro domain,” or “IGF-1-Pro-Modified” as used herein refers to the pro-peptide of IGF-1 which is a naturally occurring amino acid sequence which does not have the function of a signal peptide in nature which is referred to in the Uniprot database as P05019 and in the Genbank database as NM_000618.4, NM_001111284.1 and NM_001111285.2 is modified by deletion of ten amino acid residues (VKMHTMSSSH (SEQ ID NO: 198)) flanking 22-31 in the N-terminal end of pro peptide and has preferably the amino acid sequence as shown in SEQ ID NO: 22 and/or is preferably encoded by the DNA sequence as shown in SEQ ID NO: 23.

SEQ ID NO: 22
Met-Leu-Phe-Tyr-Leu-Ala-Leu-Cys-Leu-Leu-Thr-
Phe-Thr-Ser-Ser-Ala-Thr-Ala
SEQ ID NO: 23
ATGCTGTTCTATCTGGCCCTGTGCCTGCTGACCTTTACCAGCTCTGC
TACCGCC

The term “the mRNA comprises a nucleic acid sequence encoding the propeptide of IGF-1, and a nucleic acid sequence encoding the mature IGF-1 and does not comprise a nucleic acid sequence encoding an E-peptide of IGF-1” as used herein refers usually to a mRNA which comprises a nucleotide sequence encoding the propeptide (also called pro-domain) of human IGF-1 having 27 amino acids, and a nucleotide sequence encoding the mature human IGF-1 having 70 amino acids and which does not comprise a nucleotide sequence encoding an E-peptide (also called E-domain) of human IGF-1 i.e., does not comprise a nucleotide sequence encoding a Ea-, Eb-, or Ec-domain. The nucleotide sequence encoding the propeptide (also called pro-domain) of human IGF-1 having 27 amino acids, and the nucleotide sequence encoding the mature human IGF-1 having 70 amino acids may be codon-optimized.

The term “hydrophobic score” or “hydrophobicity score” is used synonymously to the term “hydropathy score” herein and refers to the degree of hydrophobicity of an amino acid as calculated according to the Kyte-Doolittle scale (Kyte J., Doolittle R. F.; J. Mol. Biol. 157:105-132(1982)). The amino acid hydrophobic scores according to the Kyte-Doolittle scale are as follows:

| Amino Acid | One Letter Code | Hydrophobic Score |
|---|---|---|
| Isoleucine | I | 4.5 |
| Valine | V | 4.2 |
| Leucine | L | 3.8 |
| Phenylalanine | F | 2.8 |
| Cysteine | C | 2.5 |
| Methionine | M | 1.9 |
| Alanine | A | 1.8 |
| Glycine | G | −0.4 |
| Threonine | T | −0.7 |
| Serine | S | −0.8 |
| Tryptophan | W | −0.9 |
| Tyrosine | Y | −1.3 |
| Proline | P | −1.6 |
| Histidine | H | −3.2 |
| Glutamic acid | E | −3.5 |
| Glutamine | Q | −3.5 |
| Aspartic acid | D | −3.5 |
| Asparagine | N | −3.5 |
| Lysine | K | −3.9 |
| Arginine | R | −4.5 |

The “average hydrophobic score” of an amino acid sequence e.g., the average hydrophobic score of the amino acids 1-9 of the N-terminal end of the amino acid sequence of a signal peptide is calculated by adding the hydrophobic score according to the Kyte-Doolittle scale of each of the amino acid of the amino acid sequence e.g., the hydrophobic score of each of the nine amino acids of the amino acids 1-9 of the N-terminal end, divided by the number of the amino acids, e.g., divided by nine.

The polarity is calculated according to Zimmerman Polarity index (Zimmerman J. M., Eliezer N., Simha R.; J. Theor. Biol. 21:170-201(1968)). The “average polarity” of an amino acid sequence e.g., the average polarity of the amino acids 1-9 of the N-terminal end of the amino acid sequence of a signal peptide is calculated by adding the polarity value calculated according to Zimmerman Polarity index of each of the amino acid of the amino acid sequence e.g., the average polarity of each of the nine amino acids of the amino acids 1-9 of the N-terminal end, divided by the number of the amino acids, e.g., divided by nine. The polarity of amino acids according to Zimmerman Polarity index is as follows:

| Amino Acid | One Letter Code | Polarity |
|---|---|---|
| Isoleucine | I | 0.13 |
| Valine | V | 0.13 |
| Leucine | L | 0.13 |
| Phenylalanine | F | 0.35 |
| Cysteine | C | 1.48 |
| Methionine | M | 1.43 |
| Alanine | A | 0 |
| Glycine | G | 0 |
| Threonine | T | 1.66 |
| Serine | S | 1.67 |
| Tryptophan | W | 2.1 |
| Tyrosine | Y | 1.61 |
| Proline | P | 1.58 |
| Histidine | H | 51.6 |
| Glutamic acid | E | 49.9 |
| Glutamine | Q | 3.53 |
| Aspartic acid | D | 49.7 |
| Asparagine | N | 3.38 |
| Lysine | K | 49.5 |
| Arginine | R | 52 |

### Disease and Treatment

In some aspects, provided herein, is a cell comprising the composition of any recombinant polynucleic acid or RNA constructs described herein. In some aspects, provided herein, is a pharmaceutical composition comprising the composition of any recombinant polynucleic acid or RNA constructs described herein and a pharmaceutically acceptable excipient. Pharmaceutical compositions can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, the pharmaceutical composition facilitates administration of the compound to an organism.

In some aspects, provided herein, is the pharmaceutical composition, the cell, the recombinant polynucleic acid construct, or the recombinant RNA construct described herein for use a medicament. In some aspects, provided herein, is a method of treating a disease or a condition in a subject in need thereof, comprising administering to the subject the pharmaceutical composition, the cell, the recombinant polynucleic acid construct, or the recombinant RNA construct, described herein. In some aspects, provided herein, is the pharmaceutical composition, the cell, the recombinant polynucleic acid construct, or the recombinant RNA construct described herein for use in a method of treating a disease or a condition in a subject in need thereof. In some aspects, provided herein, is the use of the pharmaceutical composition, the cell, the recombinant polynucleic acid construct, or the recombinant RNA construct described herein for the manufacture of a medicament for treating a disease or a condition in a subject in need thereof. In some embodiments, the disease or the condition is selected from the group consisting of SARS (severe acute respiratory syndrome), acute respiratory distress syndrome (ARDS), venous thromboembolism, cardiovascular complications, acute kidney injury, acute liver injury, neurologic complications, cytokine release syndrome, pediatric multisystem inflammatory syndrome, septic shock, disseminated intravascular coagulation, acute respiratory failure, and any combination thereof, intervertebral disc disease (IVDD), osteoarthritis, psoriasis, fibrodysplasia ossificans progressiva (FOP), and amyotrophic lateral sclerosis (ALS). In some embodiments, the disease or the condition is selected from the group consisting of SARS (severe acute respiratory syndrome), acute respiratory distress syndrome (ARDS), venous thromboembolism, cardiovascular complications, acute kidney injury, acute liver injury, neurologic complications, cytokine release syndrome, pediatric multisystem inflammatory syndrome, septic shock, disseminated intravascular coagulation, acute respiratory failure, and any combination thereof, intervertebral disc disease (IVDD), osteoarthritis, and psoriasis. In some embodiments, the disease or the condition is selected from the group consisting of SARS (severe acute respiratory syndrome), intervertebral disc disease (IVDD), osteoarthritis, psoriasis, fibrodysplasia ossificans progressiva (FOP), and amyotrophic lateral sclerosis (ALS). In some embodiments, the disease or the condition is selected from the group consisting of SARS (severe acute respiratory syndrome), intervertebral disc disease (IVDD), osteoarthritis, and psoriasis.

In some embodiments, the disease or the condition is selected from the group consisting of intervertebral disc disease (IVDD), osteoarthritis, psoriasis, fibrodysplasia ossificans progressiva (FOP), and amyotrophic lateral sclerosis (ALS). In some embodiments, the disease or the condition is selected from the group consisting of intervertebral disc disease (IVDD), osteoarthritis, and psoriasis. In some embodiments, the disease or condition comprises a skin disease or condition. In some embodiments, the skin disease or condition comprises an inflammatory skin disorder. In some embodiments, an inflammatory skin disorder comprises psoriasis. In some embodiments, the disease or condition comprises a muscular disease or condition. In some embodiments, the muscular disease or condition comprises a skeletal muscle disorder. In some embodiments, the skeletal muscle disorder comprises fibrodysplasia ossificans progressiva (FOP). In some embodiments, the disease or condition comprises a neurodegenerative disease or condition. In some embodiments, the neurodegenerative disease or condition comprises a motor neuron disorder. In some embodiments, the motor neuron disorder comprises amyotrophic lateral sclerosis (ALS). In some embodiments, the disease or condition comprises a joint disease or condition. In some embodiments, the joint disease or condition comprises a joint degeneration. In some embodiments, the joint degeneration comprises intervertebral disc disease (IVDD) or osteoarthritis (OA).

Intervertebral disc disease (IVDD) is a condition that is estimated to affect about 5% of the population in developed countries each year and characterized by the degeneration of one or more of the discs that separate each vertebra of the spine. The intervertebral discs provide cushioning between vertebrae and absorb pressure put on the spine. Although discs in the lower region of the spine are most often affected in IVDD, any part of the spine can have disc degeneration and thus, this condition causes pain in the back, neck, legs, and arms. Also, depending on the location of the affected disc or discs, IVDD can cause periodic or chronic pain, which can be worse when sitting, bending, twisting, or lifting object. IVDD results from a combination of genetic and environmental factors, most of which remain unknown. Several genes have been identified to have variations that may influence the risk of developing IVDD and these include genes associated with collagen, immune function, and proteins that play roles in the development and maintenance of the intervertebral discs and vertebrae. Nongenetic factors include aging, smoking, obesity, chronic inflammation, and driving for a long period of time. Two of these genes are Insulin-like growth factor 1 (IGF-1) and its receptor (insulin-like growth factor 1 receptor, IGF-1R), which can regulate the extracellular matrix synthesis and play a crucial role in maintaining the normal functions of the intervertebral disc.

Osteoarthritis is a common disease of the joints, characterized by progressive degeneration of articular cartilage, causing pain, stiffness, and restricted movement as the condition gets worse. Areas of bone no longer cushioned by cartilage rub against each other and start to break down, causing further damage such as inflammation as the immune system attempts to repair and rebuild these tissues. In addition, osteophytes (or abnormal growths of bone and other tissue) can also occur and these may be visible as enlarged joints. It is thought that the balance of catabolism and anabolism is lost in osteoarthritis patients, leading to cartilage damage and complete breakdown. The genes of which expression affects osteoarthritis risk are typically involved in the formation and maintenance of bone and cartilage.

In both IVDD and osteoarthritis, decreasing inflammation (e.g., decreasing IL-1 beta, IL-8, etc.) while increasing anabolic signal (e.g., IGF-1, etc.) could have a therapeutic effect. In some aspects, provided herein, is a method of treating intervertebral disc disease (IVDD) in a subject, the method comprising administering to the subject the pharmaceutical composition, the cell, the recombinant polynucleic acid construct, or the recombinant RNA construct, comprising siRNA capable of binding to a target mRNA and an mRNA encoding a gene of interest. In a preferred embodiment, the siRNA is capable of binding to IL-1 beta mRNA. In another preferred embodiment, the siRNA is capable of binding to IL-8 mRNA. In a preferred embodiment, the mRNA encoding the gene of interest encodes IGF-1.

In some aspects, provided herein, is a method of treating a joint disease or condition in a subject, the method comprising administering to the subject the pharmaceutical composition, the cell, the recombinant polynucleic acid construct, or the recombinant RNA construct, comprising siRNA capable of binding to IL-1 beta mRNA and an mRNA encoding IGF-1. In some aspects, provided herein, is a method of treating a joint disease or condition in a subject, the method comprising administering to the subject the pharmaceutical composition comprising a recombinant polynucleic acid construct comprising a nucleic acid sequence encoding the siRNA capable of binding to IL-1 beta mRNA and a nucleic acid encoding IGF-1. In some aspects, provided herein, is a method of treating a joint disease or condition in a subject, the method comprising administering to the subject the pharmaceutical composition comprising a recombinant RNA construct comprising siRNA capable of binding to IL-1 beta mRNA and an mRNA encoding IGF-1. In some embodiments, the joint disease or condition is a joint degeneration. In some embodiments, the joint degeneration is intervertebral disc disease (IVDD) or osteoarthritis (OA).

In some aspects, provided herein, is a method of treating intervertebral disc disease (IVDD) in a subject, the method comprising administering to the subject the pharmaceutical composition, the cell, the recombinant polynucleic acid construct, or the recombinant RNA construct, comprising siRNA capable of binding to IL-1 beta mRNA and an mRNA encoding IGF-1. In some aspects, provided herein, is a method of treating intervertebral disc disease (IVDD) in a subject, the method comprising administering to the subject the pharmaceutical composition, the cell, the recombinant polynucleic acid construct, or the recombinant RNA construct, comprising siRNA capable of binding to IL-8 mRNA and an mRNA encoding IGF-1.

In some aspects, provided herein, is a method of treating intervertebral disc disease (IVDD) in a subject, the method comprising administering to the subject the pharmaceutical composition comprising a recombinant polynucleic acid construct comprising a nucleic acid sequence encoding the siRNA capable of binding to IL-1 beta mRNA and a nucleic acid encoding IGF-1. In some aspects, provided herein, is a method of treating intervertebral disc disease (IVDD) in a subject, the method comprising administering to the subject the pharmaceutical composition comprising a recombinant polynucleic acid construct comprising a nucleic acid sequence encoding the siRNA capable of binding to IL-8 mRNA and a nucleic acid sequence encoding IGF-1.

In some aspects, provided herein, is a method of treating intervertebral disc disease (IVDD) in a subject, the method comprising administering to the subject the pharmaceutical composition comprising a recombinant RNA construct comprising siRNA capable of binding to IL-1 beta mRNA and an mRNA encoding IGF-1. In some aspects, provided herein, is a method of treating intervertebral disc disease (IVDD) in a subject, the method comprising administering to the subject the pharmaceutical composition comprising a recombinant RNA construct comprising siRNA capable of binding to IL-8 mRNA and an mRNA encoding IGF-1.

In some aspects, provided herein, is a method of treating osteoarthritis in a subject, the method comprising administering to the subject the pharmaceutical composition, the cell, the recombinant polynucleic acid construct, or the recombinant RNA construct, comprising siRNA capable of binding to a target mRNA and an mRNA encoding a gene of interest. In a preferred embodiment, the siRNA is capable of binding to IL-1 beta mRNA. In another preferred embodiment, the siRNA is capable of binding to IL-8 mRNA. In a preferred embodiment, the mRNA encoding the gene of interest encodes IGF-1.

In some aspects, provided herein, is a method of treating osteoarthritis in a subject, the method comprising administering to the subject the pharmaceutical composition, the cell, the recombinant polynucleic acid construct, or the recombinant RNA construct, comprising siRNA capable of binding to IL-1 beta mRNA and an mRNA encoding IGF-1. In some aspects, provided herein, is a method of treating osteoarthritis in a subject, the method comprising administering to the subject the pharmaceutical composition, the cell, the recombinant polynucleic acid construct, or the recombinant RNA construct, comprising siRNA capable of binding to IL-8 mRNA and an mRNA encoding IGF-1.

In some aspects, provided herein, is a method of treating osteoarthritis in a subject, the method comprising administering to the subject the pharmaceutical composition comprising a recombinant polynucleic acid construct comprising a nucleic acid sequence encoding the siRNA capable of binding to IL-1 beta mRNA and a nucleic acid encoding IGF-1. In some aspects, provided herein, is a method of treating osteoarthritis in a subject, the method comprising administering to the subject the pharmaceutical composition comprising a recombinant polynucleic acid construct comprising a nucleic acid sequence encoding the siRNA capable of binding to IL-8 mRNA and a nucleic acid sequence encoding IGF-1.

In some aspects, provided herein, is a method of treating osteoarthritis in a subject, the method comprising administering to the subject the pharmaceutical composition comprising a recombinant RNA construct comprising siRNA capable of binding to IL-1 beta mRNA and an mRNA encoding IGF-1. In some aspects, provided herein, is a method of treating osteoarthritis in a subject, the method comprising administering to the subject the pharmaceutical composition comprising a recombinant RNA construct comprising siRNA capable of binding to IL-8 mRNA and an mRNA encoding IGF-1.

Psoriasis is a chronic inflammatory skin disorder, characterized by patches of red, irritated skin that are often covered by flaky white scales. Psoriasis patients may also develop psoriatic arthritis, a condition involving joint inflammation. Although the exact cause of this disease is not currently understood, the disease is thought to be an autoimmune disease caused by an immune system problem with T cells (e.g., T cells attacking healthy skin cells) and other white blood cells, such as neutrophils.

In some aspects, provided herein, is a method of treating a skin disease or condition in a subject, the method comprising administering to the subject the pharmaceutical composition, the cell, the recombinant polynucleic acid construct, or the recombinant RNA construct, comprising siRNA capable of binding to IL-1 beta mRNA and an mRNA encoding IGF-1. In some aspects, provided herein, is a method of treating a joint disease or condition in a subject, the method comprising administering to the subject the pharmaceutical composition comprising a recombinant polynucleic acid construct comprising a nucleic acid sequence encoding the siRNA capable of binding to IL-1 beta mRNA and a nucleic acid encoding IGF-1. In some aspects, provided herein, is a method of treating a skin disease or condition in a subject, the method comprising administering to the subject the pharmaceutical composition comprising a recombinant RNA construct comprising siRNA capable of binding to IL-1 beta mRNA and an mRNA encoding IGF-1. In some embodiments, the skin disease or condition is an inflammatory skin disorder. In some embodiments, the inflammatory skin disorder is psoriasis.

In some aspects, provided herein, is a method of treating psoriasis in a subject, the method comprising administering to the subject the pharmaceutical composition, the cell, the recombinant polynucleic acid construct, or the recombinant RNA construct, comprising siRNA capable of binding to a target mRNA and an mRNA encoding a gene of interest. In a preferred embodiment, the siRNA is capable of binding to IL-17 mRNA. In another embodiment, the siRNA is capable of binding to TNF-alpha mRNA. In a preferred embodiment, the mRNA encoding the gene of interest encodes IL-4.

In some aspects, provided herein, is a method of treating psoriasis in a subject, the method comprising administering to the subject the pharmaceutical composition, the cell, the recombinant polynucleic acid construct, or the recombinant RNA construct, comprising siRNA capable of binding to IL-17 mRNA and an mRNA encoding IL-4. In some aspects, provided herein, is a method of treating psoriasis in a subject, the method comprising administering to the subject the pharmaceutical composition comprising a recombinant polynucleic acid construct comprising a nucleic acid sequence encoding the siRNA capable of binding to IL-17 mRNA and a nucleic acid encoding IL-4. In some aspects, provided herein, is a method of treating psoriasis in a subject, the method comprising administering to the subject the pharmaceutical composition comprising a recombinant RNA construct comprising siRNA capable of binding to IL-17 mRNA and an mRNA encoding IL-4.

In some aspects, provided herein, is a method of treating psoriasis in a subject, the method comprising administering to the subject the pharmaceutical composition, the cell, the recombinant polynucleic acid construct, or the recombinant RNA construct, comprising siRNA capable of binding to TNF-alpha mRNA and an mRNA encoding IL-4. In some aspects, provided herein, is a method of treating psoriasis in a subject, the method comprising administering to the subject the pharmaceutical composition comprising a recombinant polynucleic acid construct comprising a nucleic acid sequence encoding the siRNA capable of binding to TNF-alpha mRNA and a nucleic acid encoding IL-4. In some aspects, provided herein, is a method of treating psoriasis in a subject, the method comprising administering to the subject the pharmaceutical composition comprising a recombinant RNA construct comprising siRNA capable of binding to TNF-alpha mRNA and an mRNA encoding IL-4.

Fibrodysplasia ossificans progressiva (FOP) is a skeletal muscle disorder in which muscle tissues and connective tissues such as tendons and ligaments are gradually ossified, forming extra-skeletal or heterotopic bones that constrains movement. The formation of extra-skeletal bone causes progressive loss of mobility as the joints become affected. Any trauma to the muscles of an individual with FOP such as a fall or an invasive medical procedure can trigger episodes of muscle swelling and inflammation followed by more rapid ossification of muscle and connective tissues in the injured area.

In some aspects, provided herein, is a method of treating a muscular disease or condition in a subject, the method comprising administering to the subject the pharmaceutical composition, the cell, the recombinant polynucleic acid construct, or the recombinant RNA construct, comprising siRNA capable of binding to ALK2 mRNA and an mRNA encoding IGF-1. In some aspects, provided herein, is a method of treating a muscular disease or condition in a subject, the method comprising administering to the subject the pharmaceutical composition comprising a recombinant polynucleic acid construct comprising a nucleic acid sequence encoding the siRNA capable of binding to ALK2 mRNA and a nucleic acid encoding IGF-1. In some aspects, provided herein, is a method of treating a muscular disease or condition in a subject, the method comprising administering to the subject the pharmaceutical composition comprising a recombinant RNA construct comprising siRNA capable of binding to ALK2 mRNA and an mRNA encoding IGF-1. In some embodiments, the muscular disease or condition is a skeletal muscle disorder. In some embodiments, the skeletal muscle disorder is fibrodysplasia ossificans progressiva (FOP).

Amyotrophic lateral sclerosis (ALS), or Lou Gehrig's disease, is a progressive neurodegenerative disease that affects nerve cells in the brain and spinal cord, causing loss of muscle. It is a motor neuron disease characterized by the degeneration of both upper and lower motor neurons, which leads to muscle weakness and eventual paralysis. The cause of ALS is not yet known, however, some biomarkers and genes associated with ALS, including Superoxide Dismutase 1 (SOD1), have been discovered. There are 2 types of ALS differentiated by genetics: familial and sporadic (idiopathic).

In some aspects, provided herein, is a method of treating a neurodegenerative disease or condition in a subject, the method comprising administering to the subject the pharmaceutical composition, the cell, the recombinant polynucleic acid construct, or the recombinant RNA construct, comprising siRNA capable of binding to SOD1 mRNA and an mRNA encoding IGF-1. In some aspects, provided herein, is a method of treating a neurodegenerative disease or condition in a subject, the method comprising administering to the subject the pharmaceutical composition comprising a recombinant polynucleic acid construct comprising a nucleic acid sequence encoding the siRNA capable of binding to SOD1 mRNA and a nucleic acid encoding IGF-1. In some aspects, provided herein, is a method of treating a neurodegenerative disease or condition in a subject, the method comprising administering to the subject the pharmaceutical composition comprising a recombinant RNA construct comprising siRNA capable of binding to SOD1 mRNA and an mRNA encoding IGF-1. In some embodiments, the neurodegenerative disease or condition is a motor neuron disorder. In some embodiments, the motor neuron disorder is amyotrophic lateral sclerosis (ALS).

In some aspects, provided herein, is a method of treating a neurodegenerative disease or condition in a subject, the method comprising administering to the subject the pharmaceutical composition, the cell, the recombinant polynucleic acid construct, or the recombinant RNA construct, comprising siRNA capable of binding to SOD1 mRNA and an mRNA encoding EPO. In some aspects, provided herein, is a method of treating a neurodegenerative disease or condition in a subject, the method comprising administering to the subject the pharmaceutical composition comprising a recombinant polynucleic acid construct comprising a nucleic acid sequence encoding the siRNA capable of binding to SOD1 mRNA and a nucleic acid encoding EPO. In some aspects, provided herein, is a method of treating a neurodegenerative disease or condition in a subject, the method comprising administering to the subject the pharmaceutical composition comprising a recombinant RNA construct comprising siRNA capable of binding to SOD1 mRNA and an mRNA encoding EPO. In some embodiments, the neurodegenerative disease or condition is a motor neuron disorder. In some embodiments, the motor neuron disorder is amyotrophic lateral sclerosis (ALS).

In some aspects, provided herein, is a method of treating a disease or a condition relating to infection with a coronavirus in a subject in need thereof, comprising administering to the subject the pharmaceutical composition, the cell, the recombinant polynucleic acid construct, or the recombinant RNA construct, described herein. In some embodiments, the disease or the condition is SARS (severe acute respiratory syndrome) caused by infection with a SARS-associated coronavirus. In some embodiments, the present invention is useful for treating a disease or condition caused by or associated with infection with a coronavirus, including but not limited to a complication of coronavirus infection. In some embodiments, the disease or condition is a respiratory syndrome, e.g., SARS (severe acute respiratory syndrome) caused by infection with a SARS-associated coronavirus. In some embodiments, the disease or condition is selected from, e.g., acute respiratory distress syndrome (ARDS), venous thromboembolism, cardiovascular complications, acute kidney injury, acute liver injury, neurologic complications, cytokine release syndrome, pediatric multisystem inflammatory syndrome, septic shock, disseminated intravascular coagulation, acute respiratory failure, and any combination thereof. In some embodiments, the disease or condition associated with coronavirus infection treated using the compositions or methods of the invention is any known to those of skill in the art and described in the literature. In some embodiments, the present invention is useful for treating such a disease or condition by parallel control and/or downregulation of a specific physiological mechanism by siRNA, and activation and/or increase of another physiological mechanism, e.g., inflammation, by overexpression of a therapeutic protein. In some embodiments, the coronavirus is SARS-CoV (also known as SARS-CoV-1; the virus responsible for 2002-2003 SARS epidemic), SARS-CoV-2 (the virus that causes novel coronavirus disease-2019, or COVID-19), or MERS-CoV (Middle East Respiratory Syndrome virus). In some embodiments, one or more of SARS-CoV, SARS-CoV-2, and MERS is treated using the present invention. These and related viruses are described by, e.g., Coronaviridae Study Group of the International Committee on Taxonomy of Viruses, March 2020, Nature Microbiology 5:536-44), incorporated herein by reference.

In some aspects, provided herein, is a method of treating a disease or a condition relating to infection with a coronavirus in a subject in need thereof, comprising administering to the subject the pharmaceutical composition, the cell, the recombinant polynucleic acid construct, or the recombinant RNA construct described herein.

In some aspects, the composition administered to the subject comprises a polynucleic acid construct encoding or comprising: (i) at least one siRNA capable of binding to an IL-6 mRNA; and (ii) an mRNA IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct comprises 1 siRNA directed to an IL-6 mRNA. In related aspects, the polynucleic acid construct comprises 3 siRNAs, each directed to an IL-6 mRNA. In related aspects, each of the at least 3 siRNAs is the same, different, or a combination thereof. In related aspects, the polynucleic acid construct comprises a sequence as set forth in SEQ ID NO: 29 or 30 (Compound B1 or B2).

In some aspects, the composition administered to the subject comprises a polynucleic acid construct encoding or comprising: (i) at least one siRNA capable of binding to an Interleukin 6R (IL-6R) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R mRNA. In related aspects, the polynucleic acid construct comprises 3 siRNAs, each directed to an IL-6R mRNA. In related aspects, each of the at least 3 siRNAs is the same, different, or a combination thereof. In related aspects, the polynucleic acid construct comprises a sequence as set forth in SEQ ID NO: 31 (Compound B3).

In some aspects, the composition administered to the subject comprises a polynucleic acid construct encoding or comprising: (i) at least one siRNA capable of binding to an Interleukin 6R alpha (IL-6R-alpha) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R-alpha mRNA. In related aspects, the polynucleic acid construct encodes or comprises 3 siRNAs, each directed to an IL-6R-alpha mRNA. In related aspects, each of the at least 3 siRNAs is the same, different, or a combination thereof. In related aspects, the polynucleic acid construct comprises a sequence as set forth in SEQ ID NO: 32 (Compound B4).

In some aspects, the composition administered to the subject comprises a polynucleic acid construct encoding or comprising: (i) at least one siRNA capable of binding to an Interleukin 6R beta (IL-6R-beta) mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an IL-6R-beta mRNA. In related aspects, the polynucleic acid construct encodes or comprises 3 siRNAs, each directed to an IL-6R-beta mRNA. In related aspects, each of the at least 3 siRNAs is the same, different, or a combination thereof. In related aspects, the polynucleic acid construct comprises a sequence as set forth in SEQ ID NO: 33 (Compound B5).

In some aspects, the composition administered to the subject comprises a polynucleic acid construct encoding or comprising: (i) at least one siRNA capable of binding to an ACE2 mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an ACE2 mRNA. In related aspects, the polynucleic acid construct encodes or comprises 3 siRNAs, each directed to an ACE2 mRNA. In related aspects, each of the at least 3 siRNAs is the same, different, or a combination thereof. In related aspects, the polynucleic acid construct comprises a sequence as set forth in SEQ ID NO: 34 or 35 (Compound B6 or B7).

In some aspects, the composition administered to the subject comprises a recombinant polynucleic acid construct encoding or comprising: (i) at least one siRNA capable of binding to a SARS CoV-2 ORF1ab mRNA, at least one siRNA capable of binding to a SARS CoV-2 S mRNA, at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to a SARS CoV-2 ORF1ab mRNA, one directed to a SARS CoV-2 S mRNA, and one directed to a SARS CoV-2 N mRNA. In related aspects, each of the at least 3 siRNAs is the same, different, or a combination thereof. In certain aspects, such a composition, including a composition comprising Compound B8 (SEQ ID NO: 36) is contemplated for use in methods described herein, e.g., for modulating or regulating gene expression in relation to infection with SARS CoV, SARS CoV-2, or both. In related aspects, the polynucleic acid construct comprises a sequence as set forth in SEQ ID NO: 36.

In some aspects, the composition administered to the subject comprises a polynucleic acid construct encoding or comprising: (i) at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the polynucleic acid construct comprises a sequence as set forth in SEQ ID NO: 37 or 39 (Compound B9 or B11).

In some aspects, the composition administered to the subject comprises a polynucleic acid construct encoding or comprising: (i) at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 N mRNA. In related aspects, the polynucleic acid construct encodes or comprises 3 siRNAs, each directed to a SARS CoV-2 N mRNA. In related aspects, each of the at least 3 siRNAs is the same, different, or a combination thereof. In related aspects, the polynucleic acid construct comprises a sequence as set forth in SEQ ID NO: 38 (Compound B10).

In some aspects, the composition administered to the subject comprises a polynucleic acid construct encoding or comprising: (i) at least one siRNA capable of binding to a SARS CoV-2 ORF1ab mRNA; and (ii) an mRNA encoding IFN-beta. In related aspects, the mRNA of ii) encodes or further encodes an ACE2 soluble receptor. In related aspects, the polynucleic acid construct encodes or comprises 1 siRNA directed to a SARS CoV-2 ORF1ab mRNA. In related aspects, the polynucleic acid construct encodes or comprises 3 siRNAs, each directed to a SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS. In related aspects, the polynucleic acid construct comprises a sequence as set forth in any one of SEQ ID NOs: 40, 41 and 42 (Compounds B12, B13 and B14).

In some aspects, the composition administered to the subject comprises a polynucleic acid construct encoding or comprising: (i) at least one siRNA capable of binding to an IL-6 mRNA, at least one siRNA capable of binding to an ACE2 mRNA, and at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an IL-6 mRNA, one directed to an ACE2 mRNA, and one directed to a SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the polynucleic acid construct comprises a sequence as set forth in any one of SEQ ID NOs: 43, 44, and 45 (Compounds B15, B16, and B17).

In some aspects, the composition administered to the subject comprises a polynucleic acid construct encoding or comprising: (i) at least one small interfering RNA capable of binding to a SARS CoV-2 ORF1ab mRNA, at least one siRNA capable of binding to a SARS CoV-2 S mRNA, and at least one siRNA capable of binding to a SARS CoV-2 N mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an ORF1ab mRNA, one directed to a SARS CoV-2 S mRNA, and one directed to a SARS CoV-2 N mRNA. In related aspects, the polynucleic acid construct comprises a sequence as set forth in SEQ ID NO: 46 (Compound B18). In related aspects, the polynucleic acid construct comprises a sequence as set forth in SEQ ID NO: 190 (Compound B18).

In some aspects, the composition administered to the subject comprises a polynucleic acid construct encoding or comprising: (i) at least one siRNA capable of binding to a SARS CoV-2 S mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 47 (Compound B19).

In some aspects, the present invention provides a composition comprising a recombinant polynucleic acid construct comprising a nucleic acid sequence selected from the group consisting of SEQ ID NOs: 29-47. In some aspects, the present invention provides a composition comprising a recombinant polynucleic acid construct comprising a nucleic acid sequence of SEQ ID NO: 190.

The compositions of the present invention can be administered to a subject using any suitable methods known in the art. Suitable formulations for use in the present invention and methods of delivery are generally well known in the art. For example, the compositions described herein can be administered to the subject in a variety of ways, including parenterally, intravenously, intradermally, intramuscularly, colonically, rectally, or intraperitoneally. In some embodiments, the compositions described herein is administered by intraperitoneal injection, intramuscular injection, subcutaneous injection, or intravenous injection of the subject. In some embodiments, the compositions described herein can be administered parenterally, intravenously, intramuscularly or orally.

Any of the compositions of the present invention may be provided together with an instruction manual. The instruction manual may comprise guidance for the skilled person or attending physician how to treat (or prevent) a disease or a disorder as described herein (e.g., IVDD, osteoarthritis, psoriasis, or skeletal muscle injury) in accordance with the present invention. In some embodiments, the instruction manual may comprise guidance as to the herein described mode of delivery/administration and delivery/administration regimen, respectively (e.g., route of delivery/administration, dosage regimen, time of delivery/administration, frequency of delivery/administration, etc.). In some embodiments, the instruction manual may comprise the instruction that how the composition of the present invention is to be administrated or injected and/or is prepared for administration or injection. In principle, what has been described herein elsewhere with respect to the mode of delivery/administration and delivery/administration regimen, respectively, may be comprised as respective instructions in the instruction manual.

The composition of the present invention can be used in a gene therapy. In certain some embodiments, the composition comprising the recombinant polynucleic acid or RNA construct described herein can be delivered to a cell in gene therapy vectors. Gene therapy vectors and methods of gene delivery are well known in the art. Non-limiting examples of these methods include viral vector delivery systems including DNA and RNA viruses, which have either episomal or integrated genomes after delivery to the cell, non-viral vector delivery systems including DNA plasmids, naked nucleic acid, and nucleic acid complexed with a delivery vehicle, transposon system (for delivery and integration into the host genomes; Moriarity, et al. (2013) Nucleic Acids Res 41(8), e92, Aronovich, et al., (2011) Hum. Mol. Genet. 20(R1), R14-R20), retrovirus-mediated DNA transfer (e.g., Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus; see e.g., Kay et al. (1993) Science 262, 117-119, Anderson (1992) Science 256, 808-813), and DNA virus-mediated DNA transfer including adenovirus, herpes virus, parvovirus and adeno-associated virus (e.g., Ali et al. (1994) Gene Therapy 1, 367-384). Viral vectors also include but are not limited to adeno-associated virus, adenoviral virus, lentivirus, retroviral, and herpes simplex virus vectors. Vectors capable of integration in the host genome include but are not limited to retrovirus or lentivirus.

In some embodiments, the composition comprising the recombinant polynucleic acid or RNA construct described herein can be delivered to a cell via direct DNA transfer (Wolff et al. (1990) Science 247, 1465-1468). The recombinant polynucleic acid or RNA construct can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10(4), e0118803). In another embodiment, the composition comprising the recombinant polynucleic acid or RNA construct described herein can be delivered to a cell via liposome-mediated DNA transfer (e.g., Gao & Huang (1991) Biochem. Biophys. Res. Comm. 179, 280-285, Crystal (1995) Nature Med. 1, 15-17, Caplen et al. (1995) Nature Med. 3, 39-46). The term “liposome” can encompass a variety of single and multilamellar lipid vehicles formed by the generation of enclosed lipid bilayers or aggregates. The recombinant polynucleic acid or RNA construct can be encapsulated in the aqueous interior of a liposome, interspersed within the lipid bilayer of a liposome, attached to a liposome via a linking molecule that is associated with both the liposome and the oligonucleotide, entrapped in a liposome, or complexed with a liposome.

### Modulation of Gene Expression

In some aspects, provided herein, is a method of simultaneously expressing an siRNA and an mRNA from a single RNA transcript in a cell, comprising introducing into the cell the composition of any recombinant polynucleic acid constructs described herein.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest, and wherein the expression of the target mRNA and the gene of interest is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-1 beta mRNA; and (ii) at least one nucleic acid sequence encoding IGF-1; wherein the expression of the IL-1 beta mRNA and the IGF-1 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-8 mRNA; and (ii) at least one nucleic acid sequence encoding IGF-1; wherein the expression of the IL-8 mRNA and the IGF-1 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-17 mRNA; and (ii) at least one nucleic acid sequence encoding IL-4; wherein the expression of the IL-17 mRNA and the IL-4 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a TNF-alpha mRNA; and (ii) at least one nucleic acid sequence encoding IL-4; wherein the expression of the TNF-alpha mRNA and the IL-4 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a TNF-alpha mRNA and at least one nucleic acid sequence encoding or comprising a small interfering RNA (siRNA) capable of binding to a IL-17 mRNA; and (ii) at least one nucleic acid sequence encoding IL-4; wherein the expression of the TNF-alpha mRNA, the IL-17 mRNA and the IL-4 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to an ALK2 mRNA; and (ii) at least one nucleic acid sequence encoding IGF-1; wherein the expression of the ALK2 mRNA and the IGF-1 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a SOD1 mRNA; and (ii) at least one nucleic acid sequence encoding IGF-1; wherein the expression of the SOD1 mRNA and the IGF-1 is modulated simultaneously.

In some aspects, provided herein, is a method of simultaneously modulating the expression of two or more genes in a cell, comprising introducing into the cell a recombinant polynucleic acid construct comprising: (i) at least one nucleic acid sequence encoding or comprising a small interfering RNA (siRNA) capable of binding to a SOD1 mRNA; and (ii) at least one nucleic acid sequence encoding EPO; wherein the expression of the SOD1 mRNA and the EPO is modulated simultaneously.

In some aspects, provided herein, is a method of simultaneously modulating the expression of two or more genes in a cell, comprising introducing into the cell a recombinant polynucleic acid construct encoding or comprising: (i) at least one siRNA capable of binding to an IL-6 mRNA; and (ii) an mRNA IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct comprises 1 siRNA directed to an IL-6 mRNA. In related aspects, the polynucleic acid construct comprises 3 siRNAs, each directed to an IL-6 mRNA. In related aspects, each of the at least 3 siRNAs is the same, different, or a combination thereof. In related aspects, the polynucleic acid construct comprises a sequence as set forth in SEQ ID NO: 29 or 30 (Compound B1 or B2).

In some aspects, provided herein, is a method of simultaneously modulating the expression of two or more genes in a cell, comprising introducing into the cell a recombinant polynucleic acid construct encoding or comprising: (i) at least one siRNA capable of binding to an Interleukin 6R (IL-6R) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R mRNA. In related aspects, the polynucleic acid construct comprises 3 siRNAs, each directed to an IL-6R mRNA. In related aspects, each of the at least 3 siRNAs is the same, different, or a combination thereof. In related aspects, the polynucleic acid construct comprises a sequence as set forth in SEQ ID NO: 31 (Compound B3).

In some aspects, provided herein, is a method of simultaneously modulating the expression of two or more genes in a cell, comprising introducing into the cell a recombinant polynucleic acid construct encoding or comprising: (i) at least one siRNA capable of binding to an Interleukin 6R alpha (IL-6R-alpha) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R-alpha mRNA. In related aspects, the polynucleic acid construct encodes or comprises 3 siRNAs, each directed to an IL-6R-alpha mRNA. In related aspects, each of the at least 3 siRNAs is the same, different, or a combination thereof. In related aspects, the polynucleic acid construct comprises a sequence as set forth in SEQ ID NO: 32 (Compound B4).

In some aspects, provided herein, is a method of simultaneously modulating the expression of two or more genes in a cell, comprising introducing into the cell a recombinant polynucleic acid construct encoding or comprising: (i) at least one siRNA capable of binding to an Interleukin 6R beta (IL-6R-beta) mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an IL-6R-beta mRNA. In related aspects, the polynucleic acid construct encodes or comprises 3 siRNAs, each directed to an IL-6R-beta mRNA. In related aspects, each of the at least 3 siRNAs is the same, different, or a combination thereof. In related aspects, the polynucleic acid construct comprises a sequence as set forth in SEQ ID NO: 33 (Compound B5).

In some aspects, provided herein, is a method of simultaneously modulating the expression of two or more genes in a cell, comprising introducing into the cell a recombinant polynucleic acid construct encoding or comprising: (i) at least one siRNA capable of binding to an ACE2 mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an ACE2 mRNA. In related aspects, the polynucleic acid construct encodes or comprises 3 siRNAs, each directed to an ACE2 mRNA. In related aspects, each of the at least 3 siRNAs is the same, different, or a combination thereof. In related aspects, the polynucleic acid construct comprises a sequence as set forth in SEQ ID NO: 34 or 35 (Compound B6 or B7).

In some aspects, provided herein, is a method of simultaneously modulating the expression of two or more genes in a cell, comprising introducing into the cell a recombinant polynucleic acid construct encoding or comprising: (i) at least one small interfering RNA (siRNA) capable of binding to a SARS CoV-2 ORF1ab mRNA, at least one siRNA capable of binding to a SARS CoV-2 S mRNA, at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to a SARS CoV-2 ORF1ab mRNA, one directed to a SARS CoV-2 S mRNA, and one directed to a SARS CoV-2 N mRNA. In related aspects, each of the at least 3 siRNAs is the same, different, or a combination thereof. In certain aspects, such a composition, including a composition comprising Compound B8 (SEQ ID NO: 36) is contemplated for use in methods described herein, e.g., for modulating or regulating gene expression in relation to infection with SARS CoV, SARS CoV-2, or both. In related aspects, the polynucleic acid construct comprises a sequence as set forth in SEQ ID NO: 36.

In some aspects, provided herein, is a method of simultaneously modulating the expression of two or more genes in a cell, comprising introducing into the cell a recombinant polynucleic acid construct encoding or comprising: (i) at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the polynucleic acid construct comprises a sequence as set forth in SEQ ID NO: 37 or 39 (Compound B9 or B11).

In some aspects, provided herein, is a method of simultaneously modulating the expression of two or more genes in a cell, comprising introducing into the cell a recombinant polynucleic acid construct encoding or comprising: (i) at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 N mRNA. In related aspects, the polynucleic acid construct encodes or comprises 3 siRNAs, each directed to a SARS CoV-2 N mRNA. In related aspects, each of the at least 3 siRNAs is the same, different, or a combination thereof. In related aspects, the polynucleic acid construct comprises a sequence as set forth in SEQ ID NO: 38 (Compound B10).

In some aspects, provided herein, is a method of simultaneously modulating the expression of two or more genes in a cell, comprising introducing into the cell a recombinant polynucleic acid construct encoding or comprising: (i) at least one siRNA capable of binding to a SARS CoV-2 ORF1ab mRNA; and (ii) an mRNA encoding IFN-beta. In related aspects, the mRNA of ii) encodes or further encodes an ACE2 soluble receptor. In related aspects, the polynucleic acid construct encodes or comprises 1 siRNA directed to a SARS CoV-2 ORF1ab mRNA. In related aspects, the polynucleic acid construct encodes or comprises 3 siRNAs, each directed to a SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS. In related aspects, the polynucleic acid construct comprises a sequence as set forth in any one of SEQ ID NOs: 40, 41 and 42 (Compounds B12, B13 and B14).

In some aspects, provided herein, is a method of simultaneously modulating the expression of two or more genes in a cell, comprising introducing into the cell a recombinant polynucleic acid construct encoding or comprising: (i) at least one siRNA capable of binding to an IL-6 mRNA, at least one siRNA capable of binding to an ACE2 mRNA, and at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an IL-6 mRNA, one directed to an ACE2 mRNA, and one directed to a SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the polynucleic acid construct comprises a sequence as set forth in any one of SEQ ID NOs: 43, 44, and 45 (Compounds B15, B16, and B17).

In some aspects, provided herein, is a method of simultaneously modulating the expression of two or more genes in a cell, comprising introducing into the cell a recombinant polynucleic acid construct encoding or comprising: (i) at least one small interfering RNA capable of binding to a SARS CoV-2 ORF1ab mRNA, at least one siRNA capable of binding to a SARS CoV-2 S mRNA, and at least one siRNA capable of binding to a SARS CoV-2 N mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an ORF1ab mRNA, one directed to a SARS CoV-2 S mRNA, and one directed to a SARS CoV-2 N mRNA. In related aspects, the polynucleic acid construct comprises a sequence as set forth in SEQ ID NO: 46 (Compound B18). In related aspects, the polynucleic acid construct comprises a sequence as set forth in SEQ ID NO: 190 (Compound B18).

In some aspects, provided herein, is a method of simultaneously modulating the expression of two or more genes in a cell, comprising introducing into the cell a recombinant polynucleic acid construct encoding or comprising: (i) at least one siRNA capable of binding to a SARS CoV-2 S mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 47 (Compound B19).

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a target messenger RNA (mRNA); and (ii) at least one nucleic acid sequence encoding a gene of interest; wherein the target mRNA is different from an mRNA encoded by the gene of interest, and wherein the expression of the target mRNA is downregulated and the expression of the gene of interest is upregulated simultaneously. In some embodiments, the expression of the target mRNA is downregulated by the siRNA capable of binding to the target mRNA. In some embodiments, the expression of the gene of interest is upregulated by expressing or overexpressing an mRNA or a protein encoded by the gene of interest.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-1 beta mRNA; and (ii) at least one nucleic acid sequence encoding IGF-1; wherein the expression of the IL-1 beta mRNA is downregulated and the expression of IGF-1 is upregulated simultaneously. In some embodiments, the expression of the IL-1 beta mRNA is downregulated by the siRNA capable of binding to the IL-1 beta mRNA. In some embodiments, the expression of IGF-1 is upregulated by expressing or overexpressing an IGF-1 mRNA or an IGF-1 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-8 mRNA; and (ii) at least one nucleic acid sequence encoding IGF-1; wherein the expression of the IL-8 mRNA is downregulated and the expression of IGF-1 is upregulated simultaneously. In some embodiments, the expression of the IL-8 mRNA is downregulated by the siRNA capable of binding to the IL-8 mRNA. In some embodiments, the expression of IGF-1 is upregulated by expressing or overexpressing an IGF-1 mRNA or an IGF-1 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to an IL-17 mRNA; and (ii) at least one nucleic acid sequence encoding IL-4; wherein the expression of the IL-17 mRNA is downregulated and the expression of IL-4 is upregulated simultaneously. In some embodiments, the expression of the IL-17 mRNA is downregulated by the siRNA capable of binding to the IL-17 mRNA. In some embodiments, the expression of IL-4 is upregulated by expressing or overexpressing an IL-4 mRNA or an IL-4 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a TNF-alpha mRNA; and (ii) at least one nucleic acid sequence encoding IL-4; wherein the expression of the TNF-alpha mRNA is downregulated and the expression of IL-4 is upregulated simultaneously. In some embodiments, the expression of the TNF-alpha mRNA is downregulated by the siRNA capable of binding to the TNF-alpha mRNA. In some embodiments, the expression of IL-4 is upregulated by expressing or overexpressing an IL-4 mRNA or an IL-4 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a TNF-alpha mRNA and at least one nucleic acid sequence encoding or comprising a small interfering RNA (siRNA) capable of binding to a IL-17 mRNA; and (ii) at least one nucleic acid sequence encoding IL-4; wherein the expression of the TNF-alpha mRNA and/or the expression of the IL-17 mRNA is downregulated and the expression of IL-4 is upregulated simultaneously. In some embodiments, the expression of the TNF-alpha mRNA and the expression of the IL-17 mRNA is downregulated by the siRNA capable of binding to the TNF-alpha mRNA and the siRNA capable of binding to the IL-17 mRNA. In some embodiments, the expression of IL-4 is upregulated by expressing or overexpressing an IL-4 mRNA or an IL-4 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to an ALK2 mRNA; and (ii) at least one nucleic acid sequence encoding IGF-1; wherein the expression of the ALK2 mRNA is downregulated and the expression of IGF-1 is upregulated simultaneously. In some embodiments, the expression of the ALK2 mRNA is downregulated by the siRNA capable of binding to the ALK2 mRNA. In some embodiments, the expression of IGF-1 is upregulated by expressing or overexpressing an IGF-1 mRNA or an IGF-1 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a SOD1 mRNA; and (ii) at least one nucleic acid sequence encoding IGF-1; wherein the expression of the SOD1 mRNA is downregulated and the expression of IGF-1 is upregulated simultaneously. In some embodiments, the expression of the SOD1 mRNA is downregulated by the siRNA capable of binding to the SOD1 mRNA. In some embodiments, the expression of IGF-1 is upregulated by expressing or overexpressing an IGF-1 mRNA or an IGF-1 protein.

In some aspects, provided herein, is a method of simultaneously upregulating and downregulating the expression of two or more genes in a cell, comprising introducing into the cell a recombinant polynucleic acid construct comprising: (i) at least one nucleic acid sequence encoding or comprising a small interfering RNA (siRNA) capable of binding to a SOD1 mRNA; and (ii) at least one nucleic acid sequence encoding EPO; wherein the expression of the SOD1 mRNA is downregulated and the expression of EPO is upregulated simultaneously. In some embodiments, the expression of the SOD1 mRNA is downregulated by the siRNA capable of binding to the SOD1 mRNA. In some embodiments, the expression of EPO is upregulated by expressing or overexpressing an EPO mRNA or an EPO protein.

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an IL-6 mRNA; and (ii) an mRNA IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct comprises 1 siRNA directed to an IL-6 mRNA. In related aspects, the polynucleic acid construct comprises 3 siRNAs, each directed to an IL-6 mRNA. In related aspects, each of the at least 3 siRNAs is the same, different, or a combination thereof. In related aspects, the polynucleic acid construct comprises a sequence as set forth in SEQ ID NO: 29 or 30 (Compound B1 or B2).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an Interleukin 6R (IL-6R) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R mRNA. In related aspects, the polynucleic acid construct comprises 3 siRNAs, each directed to an IL-6R mRNA. In related aspects, each of the at least 3 siRNAs is the same, different, or a combination thereof. In related aspects, the polynucleic acid construct comprises a sequence as set forth in SEQ ID NO: 31 (Compound B3).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an Interleukin 6R alpha (IL-6R-alpha) mRNA; and (ii) an mRNA encoding IFN-beta. In related aspects, the mRNA of ii) encodes or further encodes an ACE2 soluble receptor. In related aspects, the polynucleic acid construct comprises at least 1, 2, or 3 siRNAs. In related aspects, the polynucleic acid construct comprises 1 siRNA directed to an IL-6R-alpha mRNA. In related aspects, the polynucleic acid construct encodes or comprises 3 siRNAs, each directed to an IL-6R-alpha mRNA. In related aspects, each of the at least 3 siRNAs is the same, different, or a combination thereof. In related aspects, the polynucleic acid construct comprises a sequence as set forth in SEQ ID NO: 32 (Compound B4).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an Interleukin 6R beta (IL-6R-beta) mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an IL-6R-beta mRNA. In related aspects, the polynucleic acid construct encodes or comprises 3 siRNAs, each directed to an IL-6R-beta mRNA. In related aspects, each of the at least 3 siRNAs is the same, different, or a combination thereof. In related aspects, the polynucleic acid construct comprises a sequence as set forth in SEQ ID NO: 33 (Compound B5).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an ACE2 mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to an ACE2 mRNA. In related aspects, the polynucleic acid construct encodes or comprises 3 siRNAs, each directed to an ACE2 mRNA. In related aspects, each of the at least 3 siRNAs is the same, different, or a combination thereof. In related aspects, the polynucleic acid construct comprises a sequence as set forth in SEQ ID NO: 34 or 35 (Compound B6 or B7).

In some aspects, provided herein, is a method of simultaneously upregulating and downregulating the expression of two or more genes in a cell, comprising introducing into the cell a recombinant polynucleic acid construct encoding or comprising: (i) at least one small interfering RNA (siRNA) capable of binding to a SARS CoV-2 ORF1ab mRNA, at least one siRNA capable of binding to a SARS CoV-2 S mRNA, at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to a SARS CoV-2 ORF1ab mRNA, one directed to a SARS CoV-2 S mRNA, and one directed to a SARS CoV-2 N mRNA. In related aspects, each of the at least 3 siRNAs is the same, different, or a combination thereof. In certain aspects, such a composition, including a composition comprising Compound B8 (SEQ ID NO: 36) is contemplated for use in methods described herein, e.g., for modulating or regulating gene expression in relation to infection with SARS CoV, SARS CoV-2, or both. In related aspects, the polynucleic acid construct comprises a sequence as set forth in SEQ ID NO: 36.

In some aspects, provided herein, is a method of simultaneously upregulating and downregulating the expression of two or more genes in a cell, comprising introducing into the cell a recombinant polynucleic acid construct encoding or comprising: ((i) at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the polynucleic acid construct comprises a sequence as set forth in SEQ ID NO: 37 or 39 (Compound B9 or B11).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to a SARS CoV-2 N mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 N mRNA. In related aspects, the polynucleic acid construct encodes or comprises 3 siRNAs, each directed to a SARS CoV-2 N mRNA. In related aspects, each of the at least 3 siRNAs is the same, different, or a combination thereof. In related aspects, the polynucleic acid construct comprises a sequence as set forth in SEQ ID NO: 38 (Compound B10).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to a SARS CoV-2 ORF1ab mRNA; and (ii) an mRNA encoding IFN-beta. In related aspects, the mRNA of ii) encodes or further encodes an ACE2 soluble receptor. In related aspects, the polynucleic acid construct encodes or comprises 1 siRNA directed to a SARS CoV-2 ORF1ab mRNA. In related aspects, the polynucleic acid construct encodes or comprises 3 siRNAs, each directed to a SARS CoV-2 ORF1ab mRNA. In related aspects, each of the at least 3 siRNAs is the same, different, or a combination thereof. In certain aspects, such a composition, including a composition comprising Compound B12 (SEQ ID NO: 40) is contemplated for use in methods described herein, e.g., for modulating or regulating gene expression in relation to infection with SARS CoV, MERS, or both. In certain aspects, such a composition, including a composition comprising Compound B13 (SEQ ID NO: 41) is contemplated for use in methods described herein, e.g., for modulating or regulating gene expression in relation to infection with SARS CoV, SARS CoV-2, and/or MERS. In related aspects, the polynucleic acid construct comprises a sequence as set forth in any one of SEQ ID NOs: 40, 41 and 42 (Compounds B12, B13 and B14).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to an IL-6 mRNA, at least one siRNA capable of binding to an ACE2 mRNA, and at least one siRNA capable of binding to a SARS CoV-2 S mRNA; and (ii) an mRNA encoding IFN-beta. In related aspects, the mRNA of ii) encodes or further encodes an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an IL-6 mRNA, one directed to an ACE2 mRNA, and one directed to a SARS CoV-2 S mRNA. In related aspects, the mRNA encoding IFN-beta encodes the native IFN-beta signal peptide, or a modified signal peptide. In related aspects, the modified IFN-beta signal peptide is SP1 or SP2 as described herein (SEQ ID NOs: 52 and 54, respectively). In related aspects, the polynucleic acid construct comprises a sequence as set forth in any one of SEQ ID NOs: 43, 44, and 45 (Compounds B15, B16, and B17).

In some aspects, provided herein, is a method of simultaneously upregulating and downregulating the expression of two or more genes in a cell, comprising introducing into the cell a recombinant polynucleic acid construct encoding or comprising: (i) at least one small interfering RNA capable of binding to a SARS CoV-2 ORF1ab mRNA, at least one siRNA capable of binding to a SARS CoV-2 S mRNA, and at least one siRNA capable of binding to a SARS CoV-2 N mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 3 siRNAs, one directed to an ORF1ab mRNA, one directed to a SARS CoV-2 S mRNA, and one directed to a SARS CoV-2 N mRNA. In related aspects, the polynucleic acid construct comprises a sequence as set forth in SEQ ID NO: 46 (Compound B18). In related aspects, the polynucleic acid construct comprises a sequence as set forth in SEQ ID NO: 190 (Compound B18).

In some aspects, provided herein, is a method of simultaneously upregulating and downregulating the expression of two or more genes in a cell, comprising introducing into the cell a recombinant polynucleic acid construct encoding or comprising: (i) at least one siRNA capable of binding to a SARS CoV-2 S mRNA; and (ii) an mRNA encoding an ACE2 soluble receptor. In related aspects, the polynucleic acid construct encodes or comprises at least 1, 2, or 3 siRNAs. In related aspects, the polynucleic acid construct encodes or comprises 1 siRNA directed to a SARS CoV-2 S mRNA. In related aspects, the polynucleic acid construct encodes or comprises 3 siRNAs, each directed to a SARS CoV-2 S mRNA. In related aspects, each of the at least 3 siRNAs is the same, different, or a combination thereof. In related aspects, the recombinant polynucleic acid construct comprises a sequence as set forth in SEQ ID NO: 47 (Compound B19).

### EXEMPLARY EMBODIMENTS

Embodiment 1. A composition comprising a recombinant polynucleic acid construct comprising:

(i) at least one nucleic acid sequence encoding or comprising a small interfering RNA (siRNA) capable of binding to a target RNA; and
(ii) at least one nucleic acid sequence encoding a gene of interest;
wherein the target RNA is different from an mRNA encoded by the gene of interest.

Embodiment 2. The composition of embodiment 1, wherein the recombinant polynucleic acid construct comprises two or more nucleic acid sequences encoding or comprising an siRNA capable of binding to a target RNA, wherein each of the two or more nucleic acid sequences encode or comprise an siRNA capable of binding to a same target RNA or a different target RNA.

Embodiment 3. The composition of embodiment 1 or 2, wherein the target RNA is an mRNA.

Embodiment 4. The composition of embodiment 1 or 2, wherein the target RNA is an mRNA encoding a protein selected from the group consisting of: Interleukin 8 (IL-8), Interleukin 1 beta (IL-1 beta), Interleukin 17 (IL-17), and Tumor Necrosis Factor alpha (TNF-alpha).

Embodiment 5. The composition of any one of embodiments 1-4, wherein the recombinant polynucleic acid construct comprises two or more nucleic acid sequences encoding a gene of interest, wherein each of the two or more nucleic acid sequences encodes a same gene of interest or a different gene of interest.

Embodiment 6. The composition of any one of embodiments 1-5, wherein the gene of interest comprises a nucleic acid sequence encoding a protein selected from the group consisting of a secretory protein, an intracellular protein, an intraorganelle protein, and a membrane protein.

Embodiment 7. The composition of any one of embodiments 1-3, wherein the gene of interest is selected from the group consisting of Insulin-like Growth Factor 1 (IGF-1), and Interleukin 4 (IL-4).

Embodiment 8. The composition of any one of embodiments 1-7, wherein the recombinant polynucleic acid construct further comprises a nucleic acid sequence encoding a target motif operably linked to the at least one nucleic acid sequence encoding the gene of interest, wherein the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS).

Embodiment 9. The composition of embodiment 8, wherein the target motif is selected from the group consisting of:

(a) a target motif heterologous to a protein encoded by the gene of interest;
(b) a target motif heterologous to a protein encoded by the gene of interest, wherein the target motif heterologous to the protein encoded by the gene of interest is modified by insertion, deletion, and/or substitution of at least one amino acid;
(c) a target motif homologous to a protein encoded by the gene of interest, wherein the target motif homologous to the protein encoded by the gene of interest is modified by insertion, deletion, and/or substitution of at least one amino acid; and
(d) a naturally occurring amino acid sequence which does not have the function of a target motif in nature, wherein the naturally occurring amino acid sequence is optionally modified by insertion, deletion, and/or substitution of at least one amino acid.

Embodiment 10. The composition of any one of embodiments 1-9, wherein the recombinant polynucleic acid construct further comprises a nucleic acid sequence encoding or comprising a poly(A) tail, a nucleic acid sequence encoding or comprising a 5′ cap, a nucleic acid sequence encoding or comprising a promoter, or a nucleic acid sequence encoding or comprising a Kozak sequence.

Embodiment 11. The composition of any one of embodiments 1-9, wherein the recombinant polynucleic acid construct further comprises a nucleic acid sequence encoding or comprising a linker.

Embodiment 12. The composition of any one of embodiments 1-11, wherein the nucleic acid sequence encoding or comprising the linker connects (a) the at least one nucleic acid sequence encoding or comprising an siRNA capable of binding to a target mRNA and the at least one nucleic acid sequence encoding a gene of interest, (b) each of the two or more nucleic acid sequences encoding or comprising an siRNA capable of binding to a target mRNA, and/or (c) each of the two or more nucleic acid sequences encoding a gene of interest.

Embodiment 13. The composition of embodiment 11 or 12, wherein the linker comprises a tRNA linker, a 2A peptide linker or a flexible linker.

Embodiment 14. The composition of any one of embodiments 11-13, wherein nucleic acid sequence encoding or comprising the linker is at least 6 nucleic acid residues in length.

Embodiment 15. The composition of any one of embodiments 11-13, wherein the nucleic acid sequence encoding or comprising the linker is up to 50 nucleic acid residues in length.

Embodiment 16. The composition of any one of embodiments 11-13, wherein the nucleic acid sequence encoding or comprising the linker is about 6 to about 50 nucleic acid residues in length.

Embodiment 17. The composition any one of embodiments 11-13, wherein the nucleic acid sequence encoding or comprising the linker is about 6 to about 15 nucleic acid residues in length.

Embodiment 18. A composition comprising a recombinant polynucleic acid construct comprising a nucleic acid sequence selected from the group consisting of SEQ ID NOs: 1-8.

Embodiment 19. A composition comprising a recombinant RNA construct comprising:

(i) a small interfering RNA (siRNA) capable of binding to a target RNA; and
(ii) an mRNA encoding a gene of interest;
wherein the target RNA is different from the mRNA encoding the gene of interest.

Embodiment 20. The composition of embodiment 19, wherein the target RNA is mRNA.

Embodiment 21. The composition of any one of embodiments 1-20 for use in simultaneously modulating the expression of two or more genes in a cell.

Embodiment 22. The composition of any one of embodiments 1-21, wherein the at least one nucleic acid sequence encoding or comprising a small interfering RNA (siRNA) capable of binding to a target RNA (i) and the at least one nucleic acid sequence encoding a gene of interest (ii) are comprised in a sequential manner.

Embodiment 23. The composition of embodiment 22, wherein the nucleic acid sequence encoding or comprising a small interfering RNA (siRNA) capable of binding to a target RNA (i) is upstream of the at least one nucleic acid sequence encoding a gene of interest (ii).

Embodiment 24. The composition of embodiment 22, wherein the nucleic acid sequence encoding or comprising a small interfering RNA (siRNA) capable of binding to a target RNA (i) is downstream of the at least one nucleic acid sequence encoding a gene of interest (ii).

Embodiment 25. The composition of embodiment 22, wherein the nucleic acid sequence encoding or comprising a small interfering RNA (siRNA) capable of binding to a target RNA (i) is upstream or downstream of the at least one nucleic acid sequence encoding a gene of interest (ii).

Embodiment 26. The composition of any one of embodiments 1-25, wherein the siRNA capable of binding to a target RNA binds to an exon of a target mRNA.

Embodiment 27. The composition of any one of embodiments 1-26, wherein the siRNA capable of binding to a target RNA specifically binds to one target RNA.

Embodiment 28. The composition of any one of embodiments 1-27, wherein the siRNA capable of binding to a target RNA is not encoded by or comprised of an intron sequence of the gene of interest.

Embodiment 29. The composition of any one of embodiments 1-28, wherein the gene of interest is expressed without RNA splicing.

Embodiment 30. A composition comprising a recombinant polynucleic acid construct for treatment or prevention of a viral disease or condition in a subject, the construct comprising:

(i) at least one nucleic acid sequence encoding or comprising a small interfering RNA (siRNA) capable of binding to a target RNA; and
(ii) at least one nucleic acid sequence encoding a gene of interest;
wherein the target RNA is different from an mRNA encoded by the gene of interest.

Embodiment 31. The composition of embodiment 30, wherein the recombinant polynucleic acid construct comprises two or more nucleic acid sequences encoding or comprising an siRNA capable of binding to a target RNA, wherein each of the two or more nucleic acid sequences encode or comprise an siRNA capable of binding to a same target RNA or a different target RNA.

Embodiment 32. The composition of embodiment 30, wherein the recombinant polynucleic acid construct comprises three or more nucleic acid sequences encoding or comprising an siRNA capable of binding to a target RNA, wherein at least two nucleic acid sequences encode or comprise an siRNA capable of binding to the same target RNA and at least one nucleic acid sequence encodes or comprises an siRNA capable of binding to a different target RNA.

Embodiment 33. The composition of any one of embodiments 30-32, wherein the target RNA is an mRNA.

Embodiment 34. The composition of any one of embodiments 30-32, wherein the target RNA is a noncoding RNA.

Embodiment 35. The composition of any one of embodiments 30-32, wherein the target RNA is an mRNA encoding a protein selected from the group consisting of: interleukin, Angiotensin Converting Enzyme-2 (ACE2); SARS CoV-2 ORF1ab; SARS CoV-2 S, and SARS CoV-2 N.

Embodiment 36. The composition of embodiment 35, wherein the interleukin is selected from the group consisting of: IL-1alpha, IL-1beta, IL-6, IL-6R, IL-6R-alpha, interleukin IL-6R-beta, IL-18, IL-36-alpha, IL-36-beta; IL-36-gamma, and IL-33.

Embodiment 37. The composition of any one of embodiments 30-32, wherein the target RNA is an mRNA encoding a protein selected from the group consisting of: IL-6, IL-6R, IL-6R-alpha, IL-6R-beta, Angiotensin Converting Enzyme-2 (ACE2); SARS CoV-2 ORF1ab; SARS CoV-2 S, and SARS CoV-2 N.

Embodiment 38. The composition of any one of embodiments 30-37, wherein the recombinant polynucleic acid construct comprises two or more nucleic acid sequences encoding a gene of interest, wherein each of the two or more nucleic acid sequences encodes a same gene of interest or a different gene of interest.

Embodiment 39. The composition of any one of embodiments 30-38, wherein the gene of interest of (ii) is selected from the group of genes encoding: IFN alpha-n3, IFN alpha-2a, IFN alpha-2b, IFN beta-1a, IFN beta-1b, ACE2 soluble receptor, IL-37, and IL-38.

Embodiment 40. The composition of any one of embodiments 30-38, wherein the gene of interest of (ii) is selected from the group of genes encoding: IFN beta and ACE2 soluble receptor.

Embodiment 41. The composition of any one of embodiments 30-40, wherein the recombinant polynucleic acid construct further comprises a nucleic acid sequence encoding a target motif operably linked to the at least one nucleic acid sequence encoding the gene of interest, wherein the target motif comprises a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, or a centrosomal localization signal (CLS).

Embodiment 42. The composition of embodiment 41, wherein the target motif is selected from the group consisting of:

(a) a target motif heterologous to a protein encoded by the gene of interest;
(b) a target motif heterologous to a protein encoded by the gene of interest, wherein the target motif heterologous to the protein encoded by the gene of interest is modified by insertion, deletion, and/or substitution of at least one amino acid;
(c) a target motif homologous to a protein encoded by the gene of interest, wherein the target motif homologous to the protein encoded by the gene of interest is modified by insertion, deletion, and/or substitution of at least one amino acid; and
(d) a naturally occurring amino acid sequence which does not have the function of a target motif in nature, wherein the naturally occurring amino acid sequence is optionally modified by insertion, deletion, and/or substitution of at least one amino acid.

Embodiment 43. The composition of any one of embodiments 30-42, wherein the recombinant polynucleic acid construct further comprises a nucleic acid sequence encoding or comprising a poly(A) tail, a nucleic acid sequence encoding or comprising a 5′ cap, a nucleic acid sequence encoding or comprising a promoter, or a nucleic acid sequence encoding or comprising a Kozak sequence.

Embodiment 44. The composition of any one of embodiments 30-43, wherein the recombinant polynucleic acid construct further comprises a nucleic acid sequence encoding or comprising a linker.

Embodiment 45. The composition of embodiment 44, wherein the nucleic acid sequence encoding or comprising the linker connects (a) the at least one nucleic acid sequence encoding or comprising the siRNA capable of binding to the target mRNA and the at least one nucleic acid sequence encoding the gene of interest, (b) each of the two or more nucleic acid sequences encoding or comprising the siRNA capable of binding to the target mRNA, and/or (c) each of the two or more nucleic acid sequences encoding the gene of interest.

Embodiment 46. The composition of embodiment 44 or 45, wherein the linker comprises a tRNA linker, a 2A peptide linker, or a flexible linker.

Embodiment 47. The composition of any one of embodiments 44-46, wherein the nucleic acid sequence encoding or comprising the linker is at least 6 nucleic acid residues in length.

Embodiment 48. The composition of any one of embodiments 44-46, wherein the nucleic acid sequence encoding or comprising the linker is up to 50 nucleic acid residues in length.

Embodiment 49. The composition of any one of embodiments 44-46, wherein the nucleic acid sequence encoding or comprising the linker is about 6 to about 50 nucleic acid residues in length.

Embodiment 50. The composition of any one of embodiments 44-46, wherein the nucleic acid sequence encoding or comprising the linker is about 6 to about 15 nucleic acid residues in length.

Embodiment 51. A composition comprising a recombinant polynucleic acid construct comprising a nucleic acid sequence selected from the group consisting of SEQ ID NOs: 29-47.

Embodiment 55. A composition comprising a recombinant RNA construct for treatment or prevention of a viral disease or condition in a subject, the construct comprising:

(i) a small interfering RNA (siRNA) capable of binding to a target RNA; and
(ii) an mRNA encoding a gene of interest;
wherein the target RNA is different from the mRNA encoding the gene of interest.

Embodiment 53. The composition of any one of embodiments 30-52 for use in simultaneously modulating the expression of two or more genes in a cell.

Embodiment 54. The composition of any one of embodiments 30-53, wherein the composition is present in an amount sufficient to treat or prevent a viral disease or condition in the subject.

Embodiment 55. The composition of any one of embodiments 30-54, wherein the at least one nucleic acid sequence encoding or comprising a small interfering RNA (siRNA) capable of binding to a target RNA (i) and the at least one nucleic acid sequence encoding a gene of interest (ii) are comprised in a sequential manner.

Embodiment 56. The composition of embodiment 55, wherein the nucleic acid sequence encoding or comprising a small interfering RNA (siRNA) capable of binding to a target RNA (i) is upstream of the at least one nucleic acid sequence encoding a gene of interest (ii).

Embodiment 57. The composition of embodiment 55, wherein the nucleic acid sequence encoding or comprising a small interfering RNA (siRNA) capable of binding to a target RNA (i) is downstream of the at least one nucleic acid sequence encoding a gene of interest (ii).

Embodiment 58. The composition of embodiment 55, wherein the nucleic acid sequence encoding or comprising a small interfering RNA (siRNA) capable of binding to a target RNA (i) is upstream or downstream of the at least one nucleic acid sequence encoding a gene of interest (ii).

Embodiment 59. The composition of any one of embodiments 30-58, wherein the siRNA capable of binding to a target RNA binds to an exon of a target mRNA.

Embodiment 60. The composition of any one of embodiments 30-59, wherein the siRNA capable of binding to a target RNA specifically binds to one target RNA.

Embodiment 61. The composition of any one of embodiments 30-60, wherein the siRNA capable of binding to a target RNA is not encoded by or comprised of an intron sequence of the gene of interest.

Embodiment 62. The composition of any one of embodiments 30-61, wherein the gene of interest is expressed without RNA splicing.

Embodiment 63. The composition of any one of embodiments 30-62, wherein the siRNA comprises a sense strand sequence selected from SEQ ID NOs: 93-109.

Embodiment 64. The composition of any one of embodiments 1-29, wherein the siRNA comprises a sense strand sequence selected from SEQ ID NOs: 80-92.

Embodiment 65. The composition of any one of embodiments 1-29, wherein the recombinant polynucleic acid construct comprises a sequence with at least 85% sequence identity to any one of SEQ ID NOs: 177-189.

Embodiment 66. The composition of any one of embodiments 1-29, wherein the recombinant polynucleic acid construct comprises a sequence selected from the group consisting of SEQ ID NOs: 177-189.

Embodiment 67. The composition of any one of embodiments 30-63, wherein the recombinant polynucleic acid construct comprises a sequence with at least 85% sequence identity to SEQ ID NO: 190.

Embodiment 68. The composition of any one of embodiments 30-63, wherein the recombinant polynucleic acid construct comprises a sequence of SEQ ID NO: 190.

### EXAMPLES

These examples are provided for illustrative purposes only and not to limit the scope of the claims provided herein.

### Example 1: Construct Design, Sequence, and Synthesis

Construct Design

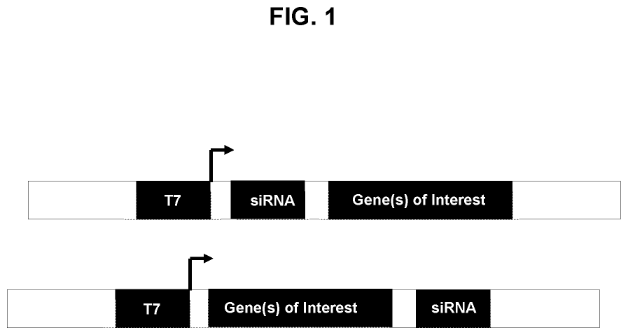

The present invention discloses that both siRNAs and any proteins of interest can be simultaneously expressed from a single transcript generated by in vitro transcription. The RNA constructs disclosed herein were designed to include siRNA designs as described in Cheng, et al. (2018) J. Mater. Chem. B., 6, 4638-4644 with one or more genes of interest downstream or upstream of the siRNA sequence (FIG. 1). The construct of the present invention may comprise more than one siRNA sequence sequentially targeting the same or different genes. Likewise, the construct of the present invention may comprise nucleic acid sequences of two or more genes of interest with a linker sequence or linker coding sequence in between (e.g., 2A peptide linker or tRNA linker).

The constructs further include T7 promoter (5′ TAATACGACTCACTATA 3′; SEQ ID NO: 25) sequence upstream of the siRNA sequence for RNA polymerase binding and successful in vitro transcription of both siRNA and the gene of interest. Alternative promoters can be utilized, and alternative promoters include SP6, T3, P60, Syn5, and KP34 promoters, which are equally functional for in vitro transcription.

Construct Synthesis

The designed constructs (Table 1, Compound ID numbers A1-A8) were gene-synthesized from GeneArt, Germany (Thermo Fisher Scientific). The constructs were synthesized as pMA-RQ vector, which contains a T7 RNA polymerase promoter, with codon optimization using GeneOptimizer algorithm. Table 1 summarizes the compounds used in the examples in the present disclosure with their respective siRNA target to downregulate protein expression, and protein target for upregulated protein expression. All uridines in Compounds A1-A8 used in the examples described herein were modified to N¹-methylpseudouridine. For each compound, the position of siRNA sequence is indicated in regard to the gene of interest. For example, “5′ siRNA position” indicates that siRNA sequences are upstream of or 5′ to the gene of interest in the compound. The sequences of the constructs of A1-A8 are shown in Table 2 and annotated as indicated in the table below.

TABLE 1
Summary of Compounds A1-A8

| | | siRNA | # of | Protein Target | |
|---|---|---|---|---|---|
| Compound ID | siRNA Target | Position | siRNAs | (gene of interest) | Indication |
| A1 | IL-8 | 5’ | 1 | IGF-1 | OA, IVDD |
| A2 | IL-8 | 5’ | 1 | IGF-1 | OA, IVDD |
| A3 | IL-8 | 5’ | 3 | IGF-1 | OA, IVDD |
| A4 | IL-8 | 5’ | 1 | — | OA, IVDD |
| A5 | IL-8 | 5’ | 3 | — | OA, IVDD |
| A6 | IL-1 beta | 5’ | 1 | IGF-1 | OA, IVDD |
| A7 | IL-1 beta | 5’ | 3 | IGF-1 | OA, IVDD |
| A8 | TNF-alpha/IL-17* | 5’ | 6 | IL-4 | Psoriasis |

OA: Osteoarthritis;
IVDD: Intervertebral disc disease;
*: only the siRNA effect of TNF-α studied

TABLE 2
Sequences of Compounds A1-A8

| SEQ ID NO: | Compound # | Sequence (5′ → 3′ direction) |
|---|---|---|
| 1 | Compound A1 | ATAGTGAGTCGTATTAACGTACCAACAACAAGGAAGTGCTAAAGAAACT |
| A1 sense | | TG [symbol] TTTATCTTAGAGGCATATCCCTGCCACCA |
| strand siRNA | | TGACCATCCTGTTTCTGACAATGGTCATCAGCTACTTCGGCTGCATGAA |
| 80, antisense | | GGCCGTGAAGATGCACACCATGAGCAGCAGCCACCTGTTCTATCTGGCC |
| 110 | | CTGTGCCTGCTGACCTTTACCAGCTCTGCTACCGCCGGACCTGAGACAC |
| | | TTTGTGGCGCTGAACTGGTGGACGCCCTGCAGTTTGTGTGTGGCGACAG |
| | | AGGCTTCTACTTCAACAAGCCCACAGGCTACGGCAGCAGCTCTAGAAGG |
| | | GCTCCTCAGACCGGAATCGTGGACGAGTGCTGCTTCAGAAGCTGCGACC |
| | | TGCGGCGGCTGGAAATGTATTGTGCCCCTCTGAAGCCTGCCAAGAGCGC |
| | | CTAATTTATCTTAGAGGCATATCCCT |
| 2 | Compound A2 | ATAGTGAGTCGTATTAACGTACCAACAACAAGGAGTGCTAAAGAAACTT |
| A2 sense | | G [symbol] TTTATCTTAGAGGCATATCCCTGCCACCATG |
| strand siRNA | | ACCATCCTGTTTCTGACAATGGTCATCAGCTACTTCGGCTGCATGAAGG |
| 81, antisense | | CCGTGAAGATGCACACCATGAGCAGCAGCCACCTGTTCTATCTGGCCCT |
| 111 | | GTGCCTGCTGACCTTTACCAGCTCTGCTACCGCCGGACCTGAGACACTT |
| | | TGTGGCGCTGAACTGGTGGACGCCCTGCAGTTTGTGTGTGGCGACAGAG |
| | | GCTTCTACTTCAACAAGCCCACAGGCTACGGCAGCAGCTCTAGAAGGGC |
| | | TCCTCAGACCGGAATCGTGGACGAGTGCTGCTTCAGAAGCTGCGACCTG |
| | | CGGCGGCTGGAAATGTATTGTGCCCCTCTGAAGCCTGCCAAGAGCGCCT |
| | | AATTTATCTTAGAGGCATATCCCT |
| 3 | Compound A3 | ATAGTGAGTCGTATTAACGTACCAACAACAAGGAGTGCTAAAGAAACTT |
| 5′ to 3′: | | G [symbol] TTTATCTTAGAGGCATATCCCTACGTACCAA |
| A3-1 sense | | CAAGAGAGTGATTGAGAGTGGACTTG [symbol] TTTAT |
| strand siRNA | | CTTAGAGGCATATCCCTACGTACCAACAAGAGAGCTCTGTCTGGACCAC |
| 81, antisense | | TTG [symbol] TTTATCTTAGAGGCATATCCCTGCCACC |
| 111; | | ATGACCATCCTGTTTCTGACAATGGTCATCAGCTACTTCGGCTGCATGA |
| A3-2 sense | | AGGCCGTGAAGATGCACACCATGAGCAGCAGCCACCTGTTCTATCTGGC |
| strand siRNA | | CCTGTGCCTGCTGACCTTTACCAGCTCTGCTACCGCCGGACCTGAGACA |
| 82, antisense | | CTTTGTGGCGCTGAACTGGTGGACGCCCTGCAGTTTGTGTGTGGCGACA |
| 112; | | GAGGCTTCTACTTCAACAAGCCCACAGGCTACGGCAGCAGCTCTAGAAG |
| A3-3 sense | | GGCTCCTCAGACCGGAATCGTGGACGAGTGCTGCTTCAGAAGCTGCGAC |
| strand siRNA | | CTGCGGCGGCTGGAAATGTATTGTGCCCCTCTGAAGCCTGCCAAGAGCG |
| 83, antisense | | CCTAATTTATCTTAGAGGCATATCCCT |
| 113 | | |
| 4 | Compound A4 | ATAGTGAGTCGTATTAACGTACCAACAACAAGGAAGTGCTAAAGAAACT |
| A4 sense | | TG [symbol] TTTATCTTAGAGGCATATCCCT |
| strand siRNA | | |
| 80, antisense | | |
| 110 | | |
| 5 | Compound A5 | ATAGTGAGTCGTATTAACGTACCAACAACAAGGAGTGCTAAAGAAACTT |
| A5-1 sense | | G [symbol] TTTATCTTAGAGGCATATCCCTACGTACCAA |
| strand siRNA | | CAAGAGAGTGATTGAGAGTGGACTTG [symbol] TTTAT |
| 81, antisense | | CTTAGAGGCATATCCCTACGTACCAACAAGAGAGCTCTGTCTGGACCAC |
| 111; | | TTG [symbol] TTTATCTTAGAGGCATATCCCT |
| A5-2 sense | | |
| strand siRNA | | |
| 82, antisense | | |
| 112; | | |
| A5-3 sense | | |
| strand siRNA | | |
| 83, antisense | | |
| 113 | | |
| 6 | Compound A6 | ATAGTGAGTCGTATTAACGTACCAACAAGAAAGATGATAAGCCCACTCT |
| A6 sense | | ACTTG [symbol] TTTATCTTAGAGGCATATCCCTG |
| strand siRNA | | CCACCATGACCATCCTGTTTCTGACAATGGTCATCAGCTACTTCGGCTG |
| 84, antisense | | CATGAAGGCCGTGAAGATGCACACCATGAGCAGCAGCCACCTGTTCTAT |
| 114 | | CTGGCCCTGTGCCTGCTGACCTTTACCAGCTCTGCTACCGCCGGACCTG |
| | | AGACACTTTGTGGCGCTGAACTGGTGGACGCCCTGCAGTTTGTGTGTGG |
| | | CGACAGAGGCTTCTACTTCAACAAGCCCACAGGCTACGGCAGCAGCTCT |
| | | AGAAGGGCTCCTCAGACCGGAATCGTGGACGAGTGCTGCTTCAGAAGCT |
| | | GCGACCTGCGGCGGCTGGAAATGTATTGTGCCCCTCTGAAGCCTGCCAA |
| | | GAGCGCCTAATTTATCTTAGAGGCATATCCCT |
| 7 | Compound A7 | ATAGTGAGTCGTATTAACGTACCAACAAGAAAGATGATAAGCCCACTCT |
| A7-1 sense | | ACTTG [symbol] TTTATCTTAGAGGCATATCCCTA |
| strand siRNA | | CGTACCAACAAGGTGATGTCTGGTCCATATGAACTTG [symbol] |
| 84, antisense | | [symbol] TTTATCTTAGAGGCATATCCCTACGTACCAACAAGATGAT |
| 114; | | AAGCCCACTCTAACTTG [symbol] TTATCTTAGAGGC |
| A7-2 sense | | ATATCCCTGCCACCATGACCATCCTGTTTCTGACAATGGTCATCAGCTA |
| strand siRNA | | CTTCGGCTGCATGAAGGCCGTGAAGATGCACACCATGAGCAGCAGCCAC |
| 85, antisense | | CTGTTCTATCTGGCCCTGTGCCTGCTGACCTTTACCAGCTCTGCTACCG |
| 115; | | CCGGACCTGAGACACTTTGTGGCGCTGAACTGGTGGACGCCCTGCAGTT |
| A7-3 sense | | TGTGTGTGGCGACAGAGGCTTCTACTTCAACAAGCCCACAGGCTACGGC |
| strand siRNA | | AGCAGCTCTAGAAGGGCTCCTCAGACCGGAATCGTGGACGAGTGCTGCT |
| 86, antisense | | TCAGAAGCTGCGACCTGCGGCGGCTGGAAATGTATTGTGCCCCTCTGAA |
| 116 | | GCCTGCCAAGAGCGCCTAATTTATCTTAGAGGCATATCCCT |
| 8 | Compound A8 | ATAGTGAGTCGTATTAACGTACCAACAAGGCGTGGAGCTGAGAGATAAA |
| A8-1 sense | | CTTG [symbol] TTTATCTTAGAGGCATATCCCTACG |
| strand siRNA | | TACCAACAAGGGCCTGTACCTCATCTACTACTTG [symbol] |
| 87, antisense | | [symbol] TTTATCTTAGAGGCATATCCCTACGTACCAACAAGGTATGAGCC |
| 117; | | CATCTATCTACTTG [symbol] TTTATCTTAGAGGCAT |
| A8-2 sense | | ATCCCTACGTACCAACAAGCAATGAGGACCCTGAGAGATACTTG [symbol] |
| strand siRNA | | [symbol] TTTATCTTAGAGGCATATCCCTACGTACCAACA |
| 88, antisense | | AGCTGATGGGAACGTGGACTAACTTG [symbol] TTT |
| 118; | | ATCTTAGAGGCATATCCCTACGTACCAACAAGGTCCTCAGATTACTACA |
| A8-3 sense | | AACTTG [symbol] TTTATCTTAGAGGCATATCCCTGC |
| strand siRNA | | CACCATGGGACTGACATCTCAACTGCTGCCTCCACTGTTCTTTCTGCTG |
| 89, antisense | | GCCTGCGCCGGCAATTTTGTGCACGGCCACAAGTGCGACATCACCCTGC |
| 119; | | AAGAGATCATCAAGACCCTGAACAGCCTGACCGAGCAGAAAACCCTGTG |
| A8-4 sense | | CACCGAGCTGACCGTGACCGATATCTTTGCCGCCAGCAAGAACACAACC |
| strand siRNA | | GAGAAAGAGACATTCTGCAGAGCCGCCACCGTGCTGAGACAGTTCTACA |
| 90, antisense | | GCCACCACGAGAAGGACACCAGATGCCTGGGAGCTACAGCCCAGCAGTT |
| 120; | | CCACAGACACAAGCAGCTGATCCGGTTCCTGAAGCGGCTGGACAGAAAT |
| A8-5 sense | | CTGTGGGGACTCGCCGGCCTGAATAGCTGCCCTGTGAAAGAGGCCAACC |
| strand siRNA | | AGTCTACCCTGGAAAACTTCCTGGAACGGCTGAAAACCATCATGCGCGA |
| 91, antisense | | GAAGTACAGCAAGTGCAGCAGCTGATTTATCTTAGAGGCATATCCCT |
| 121; | | |
| A8-6 sense | | |
| strand siRNA | | |
| 92, antisense | | |
| 122 | | |

Bold = Sense siRNA strand
Bold and Italics = anti-Sense siRNA strand
Underline = Signal peptide
Italics = Kozak sequence

TABLE 3
Plasmid Sequences for Compounds A1-A8

| SEQ | | |
|---|---|---|
| ID NO | Compound # | Sequence (5′ → 3′ direction) |
| 9 | Compound A1 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAACAAGGAAGTGCTAAAGAAACTTGTTCTTTAGCACTTCCTTGTTT |
| | | ATCTTAGAGGCATATCCCTGCCACCATGACCATCCTGTTTCTGACAATG |
| | | GTCATCAGCTACTTCGGCTGCATGAAGGCCGTGAAGATGCACACCATGA |
| | | GCAGCAGCCACCTGTTCTATCTGGCCCTGTGCCTGCTGACCTTTACCAG |
| | | CTCTGCTACCGCCGGACCTGAGACACTTTGTGGCGCTGAACTGGTGGAC |
| | | GCCCTGCAGTTTGTGTGTGGCGACAGAGGCTTCTACTTCAACAAGCCCA |
| | | CAGGCTACGGCAGCAGCTCTAGAAGGGCTCCTCAGACCGGAATCGTGGA |
| | | CGAGTGCTGCTTCAGAAGCTGCGACCTGCGGCGGCTGGAAATGTATTGT |
| | | GCCCCTCTGAAGCCTGCCAAGAGCGCCTAATTTATCTTAGAGGCATATC |
| | | CCTCTGGGCCTCATGGGCCTTCCGCTCACTGCCCGCTTTCCAGTCGGGA |
| | | AACCTGTCGTGCCAGCTGCATTAACATGGTCATAGCTGTTTCCTTGCGT |
| | | ATTGGGCGCTCTCCGCTTCCTCGCTCACTGACTCGCTGCGCTCGGTCGT |
| | | TCGGGTAAAGCCTGGGGTGCCTAATGAGCAAAAGGCCAGCAAAAGGCCA |
| | | GGAACCGTAAAAAGGCCGCGTTGCTGGCGTTTTTCCATAGGCTCCGCCC |
| | | CCCTGAGGAGCATCACAAAAATCGAGGCTCAAGTCAGAGGTGGCGAAAC |
| | | CCGACAGGACTATAAAGATACCAGGCGTTTCCCCCTGGAAGCTCCCTCG |
| | | TGCGCTCTCCTGTTCCGACCCTGCCGCTTACCGGATACCTGTCCGCCTT |
| | | TCTCCCTTCGGGAAGCGTGGCGCTTTCTCATAGCTCACGCTGTAGGTAT |
| | | CTCAGTTCGGTGTAGGTCGTTCGCTCCAAGCTGGGCTGTGTGCACGAAC |
| | | CCCCCGTTCAGCCCGACCGCTGCGCCTTATCCGGTAACTATCGTCTTGA |
| | | GTCCAACCCGGTAAGACACGACTTATCGCCACTGGCAGCAGCCACTGGT |
| | | AACAGGATTAGCAGAGCGAGGTATGTAGGCGGTGCTACAGAGTTCTTGA |
| | | AGTGGTGGCCTAACTACGGCTACACTAGAAGAACAGTATTTGGTATCTG |
| | | CGCTCTGCTGAAGCCAGTTACCTTCGGAAAAAGAGTTGGTAGCTCTTGA |
| | | TCCGGCAAACAAACCACCGCTGGTAGCGGTGGTTTTTTTGTTTGCAAGC |
| | | AGCAGATTACGCGCAGAAAAAAAGGATCTCAAGAAGATCCTTTGATCTT |
| | | TTCTACGGGGTCTGACGCTCAGTGGAACGAAAACTCACGTTAAGGGATT |
| | | TTGGTCATGAGATTATCAAAAAGGATCTTCACCTAGATCCTTTTAAATT |
| | | AAAAATGAAGTTTTAAATCAATCTAAAGTATATATGAGTAAACTTGGTC |
| | | TGACAGTTACCAATGCTTAATCAGTGAGGCACCTATCTCAGCGATCTGT |
| | | CTATTTCGTTCATCCATAGTTGCCTGACTCCCCGTCGTGTAGATAACTA |
| | | CGATACGGGAGGGCTTACCATCTGGCCCCAGTGCTGCAATGATACCGCG |
| | | AGAACCACGCTCACCGGCTCCAGATTTATCAGCAATAAACCAGCCAGCC |
| | | GGAAGGGCCGAGCGCAGAAGTGGTCCTGCAACTTTATCCGCCTCCATCC |
| | | AGTCTATTAATTGTTGCCGGGAAGCTAGAGTAAGTAGTTCGCCAGTTAA |
| | | TAGTTTGCGCAACGTTGTTGCCATTGCTACAGGCATCGTGGTGTCACGC |
| | | TCGTCGTTTGGTATGGCTTCATTCAGCTCCGGTTCCCAACGATCAAGGC |
| | | GAGTTACATGATCCCCCATGTTGTGCAAAAAAGCGGTTAGCTCCTTCGG |
| | | TCCTCCGATCGTTGTCAGAAGTAAGTTGGCCGCAGTGTTATCACTCATG |
| | | GTTATGGCAGCACTGCATAATTCTCTTACTGTCATGCCATCCGTAAGAT |
| | | GCTTTTCTGTGACTGGTGAGTACTCAACCAAGTCATTCTGAGAATAGTG |
| | | TATGCGGCGACCGAGTTGCTCTTGCCCGGCGTCAATACGGGATAATACC |
| | | GCGCCACATAGCAGAACTTTAAAAGTGCTCATCATTGGAAAACGTTCTT |
| | | CGGGGCGAAAACTCTCAAGGATCTTACCGCTGTTGAGATCCAGTTCGAT |
| | | GTAACCCACTCGTGCACCCAACTGATCTTCAGCATCTTTTACTTTCACC |
| | | AGCGTTTCTGGGTGAGCAAAAACAGGAAGGCAAAATGCCGCAAAAAAGG |
| | | GAATAAGGGCGACACGGAAATGTTGAATACTCATACTCTTCCTTTTTCA |
| | | ATATTATTGAAGCATTTATCAGGGTTATTGTCTCATGAGCGGATACATA |
| | | TTTGAATGTATTTAGAAAAATAAACAAATAGGGGTTCCGCGCACATTTC |
| | | CCCGAAAAGTGCCAC |
| 10 | Compound A2 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAACAAGGAGTGCTAAAGAAACTTGTTCTTTAGCACTCCTTGTTTAT |
| | | CTTAGAGGCATATCCCTGCCACCATGACCATCCTGTTTCTGACAATGGT |
| | | CATCAGCTACTTCGGCTGCATGAAGGCCGTGAAGATGCACACCATGAGC |
| | | AGCAGCCACCTGTTCTATCTGGCCCTGTGCCTGCTGACCTTTACCAGCT |
| | | CTGCTACCGCCGGACCTGAGACACTTTGTGGCGCTGAACTGGTGGACGC |
| | | CCTGCAGTTTGTGTGTGGCGACAGAGGCTTCTACTTCAACAAGCCCACA |
| | | GGCTACGGCAGCAGCTCTAGAAGGGCTCCTCAGACCGGAATCGTGGACG |
| | | AGTGCTGCTTCAGAAGCTGCGACCTGCGGCGGCTGGAAATGTATTGTGC |
| | | CCCTCTGAAGCCTGCCAAGAGCGCCTAATTTATCTTAGAGGCATATCCC |
| | | TCTGGGCCTCATGGGCCTTCCGCTCACTGCCCGCTTTCCAGTCGGGAAA |
| | | CCTGTCGTGCCAGCTGCATTAACATGGTCATAGCTGTTTCCTTGCGTAT |
| | | TGGGCGCTCTCCGCTTCCTCGCTCACTGACTCGCTGCGCTCGGTCGTTC |
| | | GGGTAAAGCCTGGGGTGCCTAATGAGCAAAAGGCCAGCAAAAGGCCAGG |
| | | AACCGTAAAAAGGCCGCGTTGCTGGCGTTTTTCCATAGGCTCCGCCCCC |
| | | CTGAGGAGCATCACAAAAATCGAGGCTCAAGTCAGAGGTGGCGAAACCC |
| | | GACAGGACTATAAAGATACCAGGCGTTTCCCCCTGGAAGCTCCCTCGTG |
| | | CGCTCTCCTGTTCCGACCCTGCCGCTTACCGGATACCTGTCCGCCTTTC |
| | | TCCCTTCGGGAAGCGTGGCGCTTTCTCATAGCTCACGCTGTAGGTATCT |
| | | CAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGT |
| | | CCAACCCGGTAAGACACGACTTATCGCCACTGGCAGCAGCCACTGGTAA |
| | | CAGGATTAGCAGAGCGAGGTATGTAGGCGGTGCTACAGAGTTCTTGAAG |
| | | TGGTGGCCTAACTACGGCTACACTAGAAGAACAGTATTTGGTATCTGCG |
| | | CTCTGCTGAAGCCAGTTACCTTCGGAAAAAGAGTTGGTAGCTCTTGATC |
| | | CGGCAAACAAACCACCGCTGGTAGCGGTGGTTTTTTTGTTTGCAAGCAG |
| | | CAGATTACGCGCAGAAAAAAAGGATCTCAAGAAGATCCTTTGATCTTTT |
| | | CTACGGGGTCTGACGCTCAGTGGAACGAAAACTCACGTTAAGGGATTTT |
| | | GGTCATGAGATTATCAAAAAGGATCTTCACCTAGATCCTTTTAAATTAA |
| | | AAATGAAGTTTTAAATCAATCTAAAGTATATATGAGTAAACTTGGTCTG |
| | | ACAGTTACCAATGCTTAATCAGTGAGGCACCTATCTCAGCGATCTGTCT |
| | | ATTTCGTTCATCCATAGTTGCCTGACTCCCCGTCGTGTAGATAACTACG |
| | | ATACGGGAGGGCTTACCATCTGGCCCCAGTGCTGCAATGATACCGCGAG |
| | | AACCACGCTCACCGGCTCCAGATTTATCAGCAATAAACCAGCCAGCCGG |
| | | AAGGGCCGAGCGCAGAAGTGGTCCTGCAACTTTATCCGCCTCCATCCAG |
| | | TCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTC |
| | | GTCGTTTGGTATGGCTTCATTCAGCTCCGGTTCCCAACGATCAAGGCGA |
| | | GTTACATGATCCCCCATGTTGTGCAAAAAAGCGGTTAGCTCCTTCGGTC |
| | | CTCCGATCGTTGTCAGAAGTAAGTTGGCCGCAGTGTTATCACTCATGGT |
| | | TATGGCAGCACTGCATAATTCTCTTACTGTCATGCCATCCGTAAGATGC |
| | | TTTTCTGTGACTGGTGAGTACTCAACCAAGTCATTCTGAGAATAGTGTA |
| | | TGCGGCGACCGAGTTGCTCTTGCCCGGCGTCAATACGGGATAATACCGC |
| | | GCCACATAGCAGAACTTTAAAAGTGCTCATCATTGGAAAACGTTCTTCG |
| | | GGGCGAAAACTCTCAAGGATCTTACCGCTGTTGAGATCCAGTTCGATGT |
| | | AACCCACTCGTGCACCCAACTGATCTTCAGCATCTTTTACTTTCACCAG |
| | | CGTTTCTGGGTGAGCAAAAACAGGAAGGCAAAATGCCGCAAAAAAGGGA |
| | | ATAAGGGCGACACGGAAATGTTGAATACTCATACTCTTCCTTTTTCAAT |
| | | ATTATTGAAGCATTTATCAGGGTTATTGTCTCATGAGCGGATACATATT |
| | | TGAATGTATTTAGAAAAATAAACAAATAGGGGTTCCGCGCACATTTCCC |
| | | CGAAAAGTGCCAC |
| 11 | Compound A3 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAACAAGGAGTGCTAAAGAAACTTGTTCTTTAGCACTCCTTGTTTAT |
| | | CTTAGAGGCATATCCCTACGTACCAACAAGAGAGTGATTGAGAGTGGAC |
| | | TTGCCACTCTCAATCACTCTCTTTATCTTAGAGGCATATCCCTACGTAC |
| | | CAACAAGAGAGCTCTGTCTGGACCACTTGGGTCCAGACAGAGCTCTCTT |
| | | TATCTTAGAGGCATATCCCTGCCACCATGACCATCCTGTTTCTGACAAT |
| | | GGTCATCAGCTACTTCGGCTGCATGAAGGCCGTGAAGATGCACACCATG |
| | | AGCAGCAGCCACCTGTTCTATCTGGCCCTGTGCCTGCTGACCTTTACCA |
| | | GCTCTGCTACCGCCGGACCTGAGACACTTTGTGGCGCTGAACTGGTGGA |
| | | CGCCCTGCAGTTTGTGTGTGGCGACAGAGGCTTCTACTTCAACAAGCCC |
| | | ACAGGCTACGGCAGCAGCTCTAGAAGGGCTCCTCAGACCGGAATCGTGG |
| | | ACGAGTGCTGCTTCAGAAGCTGCGACCTGCGGCGGCTGGAAATGTATTG |
| | | TGCCCCTCTGAAGCCTGCCAAGAGCGCCTAATTTATCTTAGAGGCATAT |
| | | CCCTCTGGGCCTCATGGGCCTTCCGCTCACTGCCCGCTTTCCAGTCGGG |
| | | AAACCTGTCGTGCCAGCTGCATTAACATGGTCATAGCTGTTTCCTTGCG |
| | | TATTGGGCGCTCTCCGCTTCCTCGCTCACTGACTCGCTGCGCTCGGTCG |
| | | TTCGGGTAAAGCCTGGGGTGCCTAATGAGCAAAAGGCCAGCAAAAGGCC |
| | | AGGAACCGTAAAAAGGCCGCGTTGCTGGCGTTTTTCCATAGGCTCCGCC |
| | | CCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCT |
| | | TTCTCCCTTCGGGAAGCGTGGCGCTTTCTCATAGCTCACGCTGTAGGTA |
| | | TCTCAGTTCGGTGTAGGTCGTTCGCTCCAAGCTGGGCTGTGTGCACGAA |
| | | CCCCCCGTTCAGCCCGACCGCTGCGCCTTATCCGGTAACTATCGTCTTG |
| | | AGTCCAACCCGGTAAGACACGACTTATCGCCACTGGCAGCAGCCACTGG |
| | | TAACAGGATTAGCAGAGCGAGGTATGTAGGCGGTGCTACAGAGTTCTTG |
| | | AAGTGGTGGCCTAACTACGGCTACACTAGAAGAACAGTATTTGGTATCT |
| | | GCGCTCTGCTGAAGCCAGTTACCTTCGGAAAAAGAGTTGGTAGCTCTTG |
| | | ATCCGGCAAACAAACCACCGCTGGTAGCGGTGGTTTTTTTGTTTGCAAG |
| | | CAGCAGATTACGCGCAGAAAAAAAGGATCTCAAGAAGATCCTTTGATCT |
| | | TTTCTACGGGGTCTGACGCTCAGTGGAACGAAAACTCACGTTAAGGGAT |
| | | TTTGGTCATGAGATTATCAAAAAGGATCTTCACCTAGATCCTTTTAAAT |
| | | TAAAAATGAAGTTTTAAATCAATCTAAAGTATATATGAGTAAACTTGGT |
| | | CTGACAGTTACCAATGCTTAATCAGTGAGGCACCTATCTCAGCGATCTG |
| | | TCTATTTCGTTCATCCATAGTTGCCTGACTCCCCGTCGTGTAGATAACT |
| | | ACGATACGGGAGGGCTTACCATCTGGCCCCAGTGCTGCAATGATACCGC |
| | | GAGAACCACGCTCACCGGCTCCAGATTTATCAGCAATAAACCAGCCAGC |
| | | CGGAAGGGCCGAGCGCAGAAGTGGTCCTGCAACTTTATCCGCCTCCATC |
| | | CAGTCTATTAATTGTTGCCGGGAAGCTAGAGTAAGTAGTTCGCCAGTTA |
| | | ATAGTTTGCGCAACGTTGTTGCCATTGCTACAGGCATCGTGGTGTCACG |
| | | CTCGTCGTTTGGTATGGCTTCATTCAGCTCCGGTTCCCAACGATCAAGG |
| | | CGAGTTACATGATCCCCCATGTTGTGCAAAAAAGCGGTTAGCTCCTTCG |
| | | GTCCTCCGATCGTTGTCAGAAGTAAGTTGGCCGCAGTGTTATCACTCAT |
| | | GGTTATGGCAGCACTGCATAATTCTCTTACTGTCATGCCATCCGTAAGA |
| | | TGCTTTTCTGTGACTGGTGAGTACTCAACCAAGTCATTCTGAGAATAGT |
| | | GTATGCGGCGACCGAGTTGCTCTTGCCCGGCGTCAATACGGGATAATAC |
| | | CGCGCCACATAGCAGAACTTTAAAAGTGCTCATCATTGGAAAACGTTCT |
| | | TCGGGGCGAAAACTCTCAAGGATCTTACCGCTGTTGAGATCCAGTTCGA |
| | | TGTAACCCACTCGTGCACCCAACTGATCTTCAGCATCTTTTACTTTCAC |
| | | CAGCGTTTCTGGGTGAGCAAAAACAGGAAGGCAAAATGCCGCAAAAAAG |
| | | GGAATAAGGGCGACACGGAAATGTTGAATACTCATACTCTTCCTTTTTC |
| | | AATATTATTGAAGCATTTATCAGGGTTATTGTCTCATGAGCGGATACAT |
| | | ATTTGAATGTATTTAGAAAAATAAACAAATAGGGGTTCCGCGCACATTT |
| | | CCCCGAAAAGTGCCAC |
| 12 | Compound A4 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATCAACGAGCTCATAGTGAGTCGTA |
| | | TTAACGTACCAACAACAAGGAAGTGCTAAAGAAACTTGTTCTTTAGCAC |
| | | TTCCTTGTTTATCTTAGAGGCATATCCCTGGTACCCTCTGGGCCTCATG |
| | | GGCCTTCCGCTCACTGCCCGCTTTCCAGTCGGGAAACCTGTCGTGCCAG |
| | | CTGCATTAACATGGTCATAGCTGTTTCCTTGCGTATTGGGCGCTCTCCG |
| | | CTTCCTCGCTCACTGACTCGCTGCGCTCGGTCGTTCGGGTAAAGCCTGG |
| | | GGTGCCTAATGAGCAAAAGGCGAGCAAAAGGCCAGGAACCGTAAAAAGG |
| | | CCGCGTTGCTGGCGTTTTTCCATAGGCTCCGCCCCCCTGACGAGCATCA |
| | | CAAAAATCGACGCTCAAGTCAGAGGTGGCGAAACCCGACAGGACTATAA |
| | | AGATACCAGGCGTTTCCCCCTGGAAGCTCCCTCGTGCGCTCTCCTGTTC |
| | | CGACCCTGCCGCTTACCGGATACCTGTCCGCCTTTCTCCCTTCGGGAAG |
| | | CGTGGCGCTTTCTCATAGCTCACGCTGTAGGTATCTCAGTTCGGTGTAG |
| | | GTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAG |
| | | ACACGACTTATCGCCACTGGCAGCAGCCACTGGTAACAGGATTAGCAGA |
| | | GCGAGGTATGTAGGCGGTGCTACAGAGTTCTTGAAGTGGTGGCCTAACT |
| | | ACGGCTACACTAGAAGAACAGTATTTGGTATCTGCGCTCTGCTGAAGCC |
| | | AGTTACCTTCGGAAAAAGAGTTGGTAGCTCTTGATCCGGCAAACAAACC |
| | | ACCGCTGGTAGCGGTGGTTTTTTTGTTTGCAAGCAGCAGATTACGCGCA |
| | | GAAAAAAAGGATCTCAAGAAGATCCTTTGATCTTTTCTACGGGGTCTGA |
| | | CGCTCAGTGGAACGAAAACTCACGTTAAGGGATTTTGGTCATGAGATTA |
| | | TCAAAAAGGATCTTCACCTAGATCCTTTTAAATTAAAAATGAAGTTTTA |
| | | AATCAATCTAAAGTATATATGAGTAAACTTGGTCTGACAGTTACCAATG |
| | | CTTAATCAGTGAGGCACCTATCTCAGCGATCTGTCTATTTCGTTCATCC |
| | | ATAGTTGCCTGACTCCCCGTCGTGTAGATAACTACGATACGGGAGGGCT |
| | | TACCATCTGGCCCCAGTGCTGCAATGATACCGCGAGAACCACGCTCACC |
| | | GGCTCCAGATTTATCAGCAATAAACCAGCCAGCCGGAAGGGCCGAGCGC |
| | | AGAAGTGGTCCTGCAACTTTATCCGCCTCCATCCAGTCTATTAATTGTT |
| | | GCCGGGAAGCTAGAGTAAGTAGTTCGCCAGTTAATAGTTTGCGCAACGT |
| | | TGTTGCCATTGCTACAGGCATCGTGGTGTCACGCTCGTCGTTTGGTATG |
| | | GCTTCATTCAGCTCCGGTTCCCAACGATCAAGGCGAGTTACATGATCCC |
| | | CCATGTTGTGCAAAAAAGCGGTTAGCTCCTTCGGTCCTCCGATCGTTGT |
| | | CAGAAGTAAGTTGGCCGCAGTGTTATCACTCATGGTTATGGCAGCACTG |
| | | CATAATTCTCTTACTGTCATGCCATCCGTAAGATGCTTTTCTGTGACTG |
| | | GTGAGTACTCAACCAAGTCATTCTGAGAATAGTGTATGCGGCGACCGAG |
| | | TTGCTCTTGCCCGGCGTCAATACGGGATAATACCGCGCCACATAGCAGA |
| | | ACTTTAAAAGTGCTCATCATTGGAAAACGTTCTTCGGGGCGAAAACTCT |
| | | CAAGGATCTTACCGCTGTTGAGATCCAGTTCGATGTAACCCACTCGTGC |
| | | ACCCAACTGATCTTCAGCATCTTTTACTTTCACCAGCGTTTCTGGGTGA |
| | | GCAAAAACAGGAAGGCAAAATGCCGCAAAAAAGGGAATAAGGGCGAGAC |
| | | GGAAATGTTGAATACTCATACTCTTCCTTTTTCAATATTATTGAAGCAT |
| | | TTATCAGGGTTATTGTCTCATGAGCGGATACATATTTGAATGTATTTAG |
| | | AAAAATAAACAAATAGGGGTTCCGCGCACATTTCCCCGAAAAGTGCCAC |
| 13 | Compound A5 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATGAAGGGCGCGCCAATAGTGAGTC |
| | | GTATTAACGTACCAACAACAAGGAGTGCTAAAGAAACTTGTTCTTTAGC |
| | | ACTCCTTGTTTATCTTAGAGGCATATCCCTACGTACCAACAAGAGAGTG |
| | | ATTGAGAGTGGACTTGCCACTCTCAATCACTCTCTTTATCTTAGAGGCA |
| | | TATCCCTACGTACCAACAAGAGAGCTCTGTCTGGACCACTTGGGTCCAG |
| | | ACAGAGCTCTCTTTATCTTAGAGGCATATCCCTTTTTAATTAACAACCT |
| | | GGGCCTCATGGGCCTTCCGCTCACTGCCCGCTTTCCAGTCGGGAAACCT |
| | | GTCGTGCCAGCTGCATTAACATGGTCATAGCTGTTTCCTTGCGTATTGG |
| | | GCGCTCTCCGCTTCCTCGCTCACTGACTCGCTGCGCTCGGTCGTTCGGG |
| | | TAAAGCCTGGGGTGCCTAATGAGCAAAAGGCCAGCAAAAGGCCAGGAAC |
| | | CGTAAAAAGGCCGCGTTGCTGGCGTTTTTCCATAGGCTCCGCCCCCCTG |
| | | ACGAGCATCACAAAAATCGAGGCTCAAGTCAGAGGTGGCGAAACCCGAC |
| | | AGGACTATAAAGATACCAGGCGTTTCCCCCTGGAAGCTCCCTCGTGCGC |
| | | TCTCCTGTTCCGACCCTGCCGCTTACCGGATACCTGTCCGCCTTTCTCC |
| | | CTTCGGGAAGCGTGGCGCTTTCTCATAGCTCACGCTGTAGGTATCTCAG |
| | | TTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCA |
| | | ACCCGGTAAGACACGACTTATCGCCACTGGCAGCAGCCACTGGTAACAG |
| | | GATTAGCAGAGCGAGGTATGTAGGCGGTGCTACAGAGTTCTTGAAGTGG |
| | | TGGCCTAACTACGGCTACACTAGAAGAACAGTATTTGGTATCTGCGCTC |
| | | TGCTGAAGCCAGTTACCTTCGGAAAAAGAGTTGGTAGCTCTTGATCCGG |
| | | CAAACAAACCACCGCTGGTAGCGGTGGTTTTTTTGTTTGCAAGCAGCAG |
| | | ATTACGCGCAGAAAAAAAGGATCTCAAGAAGATCCTTTGATCTTTTCTA |
| | | CGGGGTCTGACGCTCAGTGGAACGAAAACTCACGTTAAGGGATTTTGGT |
| | | CATGAGATTATCAAAAAGGATCTTCACCTAGATCCTTTTAAATTAAAAA |
| | | TGAAGTTTTAAATCAATCTAAAGTATATATGAGTAAACTTGGTCTGACA |
| | | GTTACCAATGCTTAATCAGTGAGGCACCTATCTCAGCGATCTGTCTATT |
| | | TCGTTCATCCATAGTTGCCTGACTCCCCGTCGTGTAGATAACTACGATA |
| | | CGGGAGGGCTTACCATCTGGCCCCAGTGCTGCAATGATACCGCGAGAAC |
| | | CACGCTCACCGGCTCCAGATTTATCAGCAATAAACCAGCCAGCCGGAAG |
| | | GGCCGAGCGCAGAAGTGGTCCTGCAACTTTATCCGCCTCCATCCAGTCT |
| | | ATTAATTGTTGCCGGGAAGCTAGAGTAAGTAGTTCGCCAGTTAATAGTT |
| | | TGCGCAACGTTGTTGCCATTGCTACAGGCATCGTGGTGTCACGCTCGTC |
| | | GTTTGGTATGGCTTCATTCAGCTCCGGTTCCCAACGATCAAGGCGAGTT |
| | | ACATGATCCCCCATGTTGTGCAAAAAAGCGGTTAGCTCCTTCGGTCCTC |
| | | CGATCGTTGTCAGAAGTAAGTTGGCCGCAGTGTTATCACTCATGGTTAT |
| | | GGCAGCACTGCATAATTCTCTTACTGTCATGCCATCCGTAAGATGCTTT |
| | | TCTGTGACTGGTGAGTACTCAACCAAGTCATTCTGAGAATAGTGTATGC |
| | | GGCGACCGAGTTGCTCTTGCCCGGCGTCAATACGGGATAATACCGCGCC |
| | | ACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAAC |
| | | CCACTCGTGCACCCAACTGATCTTCAGCATCTTTTACTTTCACCAGCGT |
| | | TTCTGGGTGAGCAAAAACAGGAAGGCAAAATGCCGCAAAAAAGGGAATA |
| | | AGGGCGACACGGAAATGTTGAATACTCATACTCTTCCTTTTTCAATATT |
| | | ATTGAAGCATTTATCAGGGTTATTGTCTCATGAGCGGATACATATTTGA |
| | | ATGTATTTAGAAAAATAAACAAATAGGGGTTCCGCGCACATTTCCCCGA |
| | | AAAGTGCCAC |
| 14 | Compound A6 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGAAAGATGATAAGCCCACTCTACTTGAGAGTGGGCTTATCATCT |
| | | TTCTTTATCTTAGAGGCATATCCCTGCCACCATGACCATCCTGTTTCTG |
| | | ACAATGGTCATCAGCTACTTCGGCTGCATGAAGGCCGTGAAGATGCACA |
| | | CCATGAGCAGCAGCCACCTGTTCTATCTGGCCCTGTGCCTGCTGACCTT |
| | | TACCAGCTCTGCTACCGCCGGACCTGAGACACTTTGTGGCGCTGAACTG |
| | | GTGGACGCCCTGCAGTTTGTGTGTGGCGACAGAGGCTTCTACTTCAACA |
| | | AGCCCACAGGCTACGGCAGCAGCTCTAGAAGGGCTCCTCAGACCGGAAT |
| | | CGTGGACGAGTGCTGCTTCAGAAGCTGCGACCTGCGGCGGCTGGAAATG |
| | | TATTGTGCCCCTCTGAAGCCTGCCAAGAGCGCCTAGTTTATCTTAGAGG |
| | | CATATCCCTCTGGGCCTCATGGGCCTTCCGCTCACTGCCCGCTTTCCAG |
| | | TCGGGAAACCTGTCGTGCCAGCTGCATTAACATGGTCATAGCTGTTTCC |
| | | TTGCGTATTGGGCGCTCTCCGCTTCCTCGCTCACTGACTCGCTGCGCTC |
| | | GGTCGTTCGGGTAAAGCCTGGGGTGCCTAATGAGCAAAAGGCCAGCAAA |
| | | AGGCCAGGAACCGTAAAAAGGCCGCGTTGCTGGCGTTTTTCCATAGGCT |
| | | CCGCCCCCCTGACGAGCATCACAAAAATCGACGCTCAAGTCAGAGGTGG |
| | | CGAAACCCGACAGGACTATAAAGATACCAGGCGTTTCCCCCTGGAAGCT |
| | | CCCTCGTGCGCTCTCCTGTTCCGACCCTGCCGCTTACCGGATACCTGTC |
| | | CGCCTTTCTCCCTTCGGGAAGCGTGGCGCTTTCTCATAGCTCACGCTGT |
| | | AGGTATCTCAGTTCGGTGTAGGTCGTTCGCTCCAAGCTGGGCTGTGTGC |
| | | ACGAACCCCCCGTTCAGCCCGACCGCTGCGCCTTATCCGGTAACTATCG |
| | | TCTTGAGTCCAACCCGGTAAGACACGACTTATCGCCACTGGCAGCAGCC |
| | | ACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGG |
| | | TATCTGCGCTCTGCTGAAGCCAGTTACCTTCGGAAAAAGAGTTGGTAGC |
| | | TCTTGATCCGGCAAACAAACCACCGCTGGTAGCGGTGGTTTTTTTGTTT |
| | | GCAAGCAGCAGATTACGCGCAGAAAAAAAGGATCTCAAGAAGATCCTTT |
| | | GATCTTTTCTACGGGGTCTGACGCTCAGTGGAACGAAAACTCACGTTAA |
| | | GGGATTTTGGTCATGAGATTATCAAAAAGGATCTTCACCTAGATCCTTT |
| | | TAAATTAAAAATGAAGTTTTAAATCAATCTAAAGTATATATGAGTAAAC |
| | | TTGGTCTGACAGTTACCAATGCTTAATCAGTGAGGCACCTATCTCAGCG |
| | | ATCTGTCTATTTCGTTCATCCATAGTTGCCTGACTCCCCGTCGTGTAGA |
| | | TAACTACGATACGGGAGGGCTTACCATCTGGCCCCAGTGCTGCAATGAT |
| | | ACCGCGAGAACCACGCTCACCGGCTCCAGATTTATCAGCAATAAACCAG |
| | | CCAGCCGGAAGGGCCGAGCGCAGAAGTGGTCCTGCAACTTTATCCGCCT |
| | | CCATCCAGTCTATTAATTGTTGCCGGGAAGCTAGAGTAAGTAGTTCGCC |
| | | AGTTAATAGTTTGCGCAACGTTGTTGCCATTGCTACAGGCATCGTGGTG |
| | | TCACGCTCGTCGTTTGGTATGGCTTCATTCAGCTCCGGTTCCCAACGAT |
| | | CAAGGCGAGTTACATGATCCCCCATGTTGTGCAAAAAAGCGGTTAGCTC |
| | | CTTCGGTCCTCCGATCGTTGTCAGAAGTAAGTTGGCCGCAGTGTTATCA |
| | | CTCATGGTTATGGCAGCACTGCATAATTCTCTTACTGTCATGCCATCCG |
| | | TAAGATGCTTTTCTGTGACTGGTGAGTACTCAACCAAGTCATTCTGAGA |
| | | ATAGTGTATGCGGCGACCGAGTTGCTCTTGCCCGGCGTCAATACGGGAT |
| | | AATACCGCGCCACATAGCAGAACTTTAAAAGTGCTCATCATTGGAAAAC |
| | | GTTCTTCGGGGCGAAAACTCTCAAGGATCTTACCGCTGTTGAGATCCAG |
| | | TTCGATGTAACCCACTCGTGCACCCAACTGATCTTCAGCATCTTTTACT |
| | | TTCACCAGCGTTTCTGGGTGAGCAAAAACAGGAAGGCAAAATGCCGCAA |
| | | AAAAGGGAATAAGGGCGACACGGAAATGTTGAATACTCATACTCTTCCT |
| | | TTTTCAATATTATTGAAGCATTTATCAGGGTTATTGTCTCATGAGCGGA |
| | | TACATATTTGAATGTATTTAGAAAAATAAACAAATAGGGGTTCCGCGCA |
| | | CATTTCCCCGAAAAGTGCCAC |
| 15 | Compound A7 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGAAAGATGATAAGCCCACTCTACTTGAGAGTGGGCTTATCATCT |
| | | TTCTTTATCTTAGAGGCATATCCCTACGTACCAACAAGGTGATGTCTGG |
| | | TCCATATGAACTTGTCATATGGACCAGACATCACCTTTATCTTAGAGGC |
| | | ATATCCCTACGTACCAACAAGATGATAAGCCCACTCTAACTTGTAGAGT |
| | | GGGCTTATCATCTTTATCTTAGAGGCATATCCCTGCCACCATGACCATC |
| | | CTGTTTCTGACAATGGTCATCAGCTACTTCGGCTGCATGAAGGCCGTGA |
| | | AGATGCACACCATGAGCAGCAGCCACCTGTTCTATCTGGCCCTGTGCCT |
| | | GCTGACCTTTACCAGCTCTGCTACCGCCGGACCTGAGACACTTTGTGGC |
| | | GCTGAACTGGTGGACGCCCTGCAGTTTGTGTGTGGCGACAGAGGCTTCT |
| | | ACTTCAACAAGCCCACAGGCTACGGCAGCAGCTCTAGAAGGGCTCCTCA |
| | | GACCGGAATCGTGGACGAGTGCTGCTTCAGAAGCTGCGACCTGCGGCGG |
| | | CTGGAAATGTATTGTGCCCCTCTGAAGCCTGCCAAGAGCGCCTAGTTTA |
| | | TCTTAGAGGCATATCCCTCTGGGCCTCATGGGCCTTCCGCTCACTGCCC |
| | | GCTTTCCAGTCGGGAAACCTGTCGTGCCAGCTGCATTAACATGGTCATA |
| | | GCTGTTTCCTTGCGTATTGGGCGCTCTCCGCTTCCTCGCTCACTGACTC |
| | | GCTGCGCTCGGTCGTTCGGGTAAAGCCTGGGGTGCCTAATGAGCAAAAG |
| | | GCCAGCAAAAGGCCAGGAACCGTAAAAAGGCCGCGTTGCTGGCGTTTTT |
| | | CCATAGGCTCCGCCCCCCTGACGAGCATCACAAAAATCGACGCTCAAGT |
| | | CAGAGGTGGCGAAACCCGACAGGACTATAAAGATACCAGGCGTTTCCCC |
| | | CTGGAAGCTCCCTCGTGCGCTCTCCTGTTCCGACCCTGCCGCTTACCGG |
| | | ATACCTGTCCGCCTTTCTCCCTTCGGGAAGCGTGGCGCTTTCTCATAGC |
| | | TCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGG |
| | | TAACTATCGTCTTGAGTCCAACCCGGTAAGACACGAGTTATCGCCACTG |
| | | GCAGCAGCCACTGGTAACAGGATTAGCAGAGCGAGGTATGTAGGCGGTG |
| | | CTACAGAGTTCTTGAAGTGGTGGCCTAACTACGGCTACACTAGAAGAAC |
| | | AGTATTTGGTATCTGCGCTCTGCTGAAGCCAGTTACCTTCGGAAAAAGA |
| | | GTTGGTAGCTCTTGATCCGGCAAACAAACCACCGCTGGTAGCGGTGGTT |
| | | TTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAAC |
| | | TCACGTTAAGGGATTTTGGTCATGAGATTATCAAAAAGGATCTTCACCT |
| | | AGATCCTTTTAAATTAAAAATGAAGTTTTAAATCAATCTAAAGTATATA |
| | | TGAGTAAACTTGGTCTGACAGTTACCAATGCTTAATCAGTGAGGCACCT |
| | | ATCTCAGCGATCTGTCTATTTCGTTCATCCATAGTTGCCTGACTCCCCG |
| | | TCGTGTAGATAACTACGATACGGGAGGGCTTACCATCTGGCCCCAGTGC |
| | | TGCAATGATACCGCGAGAACCACGCTCACCGGCTCCAGATTTATCAGCA |
| | | ATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAG |
| | | TAGTTCGCCAGTTAATAGTTTGCGCAACGTTGTTGCCATTGCTACAGGC |
| | | ATCGTGGTGTCACGCTCGTCGTTTGGTATGGCTTCATTCAGCTCCGGTT |
| | | CCCAACGATCAAGGCGAGTTACATGATCCCCCATGTTGTGCAAAAAAGC |
| | | GGTTAGCTCCTTCGGTCCTCCGATCGTTGTCAGAAGTAAGTTGGCCGCA |
| | | GTGTTATCACTCATGGTTATGGCAGCACTGCATAATTCTCTTACTGTCA |
| | | TGCCATCCGTAAGATGCTTTTCTGTGACTGGTGAGTACTCAACCAAGTC |
| | | ATTCTGAGAATAGTGTATGCGGCGACCGAGTTGCTCTTGCCCGGCGTCA |
| | | ATACGGGATAATACCGCGCCACATAGCAGAACTTTAAAAGTGCTCATCA |
| | | TTGGAAAACGTTCTTCGGGGCGAAAACTCTCAAGGATCTTACCGCTGTT |
| | | GAGATCCAGTTCGATGTAACCCACTCGTGCACCCAACTGATCTTCAGCA |
| | | TCTTTTACTTTCACCAGCGTTTCTGGGTGAGCAAAAACAGGAAGGCAAA |
| | | ATGCCGCAAAAAAGGGAATAAGGGCGACACGGAAATGTTGAATACTCAT |
| | | ACTCTTCCTTTTTCAATATTATTGAAGCATTTATCAGGGTTATTGTCTC |
| | | ATGAGCGGATACATATTTGAATGTATTTAGAAAAATAAACAAATAGGGG |
| | | TTCCGCGCACATTTCCCCGAAAAGTGCCAC |
| 16 | Compound A8 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGGCGTGGAGCTGAGAGATAAACTTGTTATCTCTCAGCTCCACGC |
| | | CTTTATCTTAGAGGCATATCCCTACGTACCAACAAGGGCCTGTACCTCA |
| | | TCTACTACTTGAGTAGATGAGGTACAGGCCCTTTATCTTAGAGGCATAT |
| | | CCCTACGTACCAACAAGGTATGAGCCCATCTATCTACTTGAGATAGATG |
| | | GGCTCATACCTTTATCTTAGAGGCATATCCCTACGTACCAACAAGCAAT |
| | | GAGGACCCTGAGAGATACTTGATCTCTCAGGGTCCTCATTGCTTTATCT |
| | | TAGAGGCATATCCCTACGTACCAACAAGCTGATGGGAACGTGGACTAAC |
| | | TTGTAGTCCACGTTCCCATCAGCTTTATCTTAGAGGCATATCCCTACGT |
| | | ACCAACAAGGTCCTCAGATTACTACAAACTTGTTGTAGTAATCTGAGGA |
| | | CCTTTATCTTAGAGGCATATCCCTGCCACCATGGGACTGACATCTCAAC |
| | | TGCTGCCTCCACTGTTCTTTCTGCTGGCCTGCGCCGGCAATTTTGTGCA |
| | | CGGCCACAAGTGCGACATCACCCTGCAAGAGATCATCAAGACCCTGAAC |
| | | AGCCTGACCGAGCAGAAAACCCTGTGCACCGAGCTGACCGTGACCGATA |
| | | TCTTTGCCGCCAGCAAGAACACAACCGAGAAAGAGACATTCTGCAGAGC |
| | | CGCCACCGTGCTGAGACAGTTCTACAGCCACCACGAGAAGGACACCAGA |
| | | TGCCTGGGAGCTACAGCCCAGCAGTTCCACAGACACAAGCAGCTGATCC |
| | | GGTTCCTGAAGCGGCTGGACAGAAATCTGTGGGGACTCGCCGGCCTGAA |
| | | TAGCTGCCCTGTGAAAGAGGCCAACCAGTCTACCCTGGAAAACTTCCTG |
| | | GAACGGCTGAAAACCATCATGCGCGAGAAGTACAGCAAGTGCAGCAGCT |
| | | GATTTATCTTAGAGGCATATCCCTCTGGGCCTCATGGGCCTTCCGCTCA |
| | | CTGCCCGCTTTCCAGTCGGGAAACCTGTCGTGCCAGCTGCATTAACATG |
| | | GTCATAGCTGTTTCCTTGCGTATTGGGCGCTCTCCGCTTCCTCGCTCAC |
| | | TGACTCGCTGCGCTCGGTCGTTCGGGTAAAGCCTGGGGTGCCTAATGAG |
| | | CAAAAGGCCAGCAAAAGGCCAGGAACCGTAAAAAGGCCGCGTTGCTGGC |
| | | GTTTTTCCATAGGCTCCGCCCCCCTGACGAGCATCACAAAAATCGACGC |
| | | TCAAGTCAGAGGTGGCGAAACCCGACAGGACTATAAAGATACCAGGCGT |
| | | TTCCCCCTGGAAGCTCCCTCGTGCGCTCTCCTGTTCCGACCCTGCCGCT |
| | | TACCGGATACCTGTCCGCCTTTCTCCCTTCGGGAAGCGTGGCGCTTTCT |
| | | CATAGCTCACGCTGTAGGTATCTCAGTTCGGTGTAGGTCGTTCGCTCCA |
| | | AGCTGGGCTGTGTGCACGAACCCCCCGTTCAGCCCGACCGCTGCGCCTT |
| | | ATCCGGTAACTATCGTCTTGAGTCCAACCCGGTAAGACACGACTTATCG |
| | | CCACTGGCAGCAGCCACTGGTAACAGGATTAGCAGAGCGAGGTATGTAG |
| | | GCGGTGCTACAGAGTTCTTGAAGTGGTGGCCTAACTACGGCTACACTAG |
| | | AAGAACAGTATTTGGTATCTGCGCTCTGCTGAAGCCAGTTACCTTCGGA |
| | | AAAAGAGTTGGTAGCTCTTGATCCGGCAAACAAACCACCGCTGGTAGCG |
| | | GTGGTTTTTTTGTTTGCAAGCAGCAGATTACGCGCAGAAAAAAAGGATC |
| | | TCAAGAAGATCCTTTGATCTTTTCTACGGGGTCTGACGCTCAGTGGAAC |
| | | GAAAACTCACGTTAAGGGATTTTGGTCATGAGATTATCAAAAAGGATCT |
| | | TCACCTAGATCCTTTTAAATTAAAAATGAAGTTTTAAATCAATCTAAAG |
| | | TATATATGAGTAAACTTGGTCTGACAGTTACCAATGCTTAATCAGTGAG |
| | | GCACCTATCTCAGCGATCTGTCTATTTCGTTCATCCATAGTTGCCTGAC |
| | | TCCCCGTCGTGTAGATAACTACGATACGGGAGGGCTTACCATCTGGCCC |
| | | CAGTGCTGCAATGATACCGCGAGAACCACGCTCACCGGCTCCAGATTTA |
| | | TCAGCAATAAACCAGCCAGCCGGAAGGGCCGAGCGCAGAAGTGGTCCTG |
| | | CAACTTTATCCGCCTCCATCCAGTCTATTAATTGTTGCCGGGAAGCTAG |
| | | AGTAAGTAGTTCGCCAGTTAATAGTTTGCGCAACGTTGTTGCCATTGCT |
| | | ACAGGCATCGTGGTGTCACGCTCGTCGTTTGGTATGGCTTCATTCAGCT |
| | | CCGGTTCCCAACGATCAAGGCGAGTTACATGATCCCCCATGTTGTGCAA |
| | | AAAAGCGGTTAGCTCCTTCGGTCCTCCGATCGTTGTCAGAAGTAAGTTG |
| | | GCCGCAGTGTTATCACTCATGGTTATGGCAGCACTGCATAATTCTCTTA |
| | | CTGTCATGCCATCCGTAAGATGCTTTTCTGTGACTGGTGAGTACTCAAC |
| | | CAAGTCATTCTGAGAATAGTGTATGCGGCGACCGAGTTGCTCTTGCCCG |
| | | GCGTCAATACGGGATAATACCGCGCCACATAGCAGAACTTTAAAAGTGC |
| | | TCATCATTGGAAAACGTTCTTCGGGGCGAAAACTCTCAAGGATCTTACC |
| | | GCTGTTGAGATCCAGTTCGATGTAACCCACTCGTGCACCCAACTGATCT |
| | | TCAGCATCTTTTACTTTCACCAGCGTTTCTGGGTGAGCAAAAACAGGAA |
| | | GGCAAAATGCCGCAAAAAAGGGAATAAGGGCGACACGGAAATGTTGAAT |
| | | ACTCATACTCTTCCTTTTTCAATATTATTGAAGCATTTATCAGGGTTAT |
| | | TGTCTCATGAGCGGATACATATTTGAATGTATTTAGAAAAATAAACAAA |
| | | TAGGGGTTCCGCGCACATTTCCCCGAAAAGTGCCAC |

Bold and underline = compound sequence

### Example 2: In Vitro Transcription of RNA Constructs and Data Analysis

The pMA-RQ vectors encoding Compounds A1-A8 and a homologous primer pair (Table 4) were used for PCR based in vitro transcription mRNA production. A transcription template was generated by PCR using forward and reverse primers in Table 4. The poly(A) tail was encoded in the template; the resulting PCR product encoded a 120 bp poly(A) tail (SEQ ID NO: 193). A few optimizations were made due to the repetitive sequence of siRNA flanking regions (see Tables 2 and 3) to achieve a specific amplification. These optimizations included: 1) low amount of plasmid DNA of vector; 2) use of special DNA polymerase (Q5 hot start polymerase, New England Biolabs); 3) reduced time for denaturation (30 seconds to 10 seconds) and extension (45 seconds/kb to 10 seconds/kb) for each cycle of PCR; 4) increased time for annealing (10 seconds to 30 seconds) for each cycle of PCR, and; 5) increased time for final extension (up to 15 minutes) for each cycle of PCR. In addition, to avoid non-specific primer binding, the PCR reaction mixture was prepared on ice, including thawing reagents, and the number of PCR cycles was reduced to 25.

For in vitro transcription, T7 RNA polymerase (MEGAscript kit, Thermo Fisher Scientific) was used at 37° C. for 2 hours and synthesized RNAs were chemically modified with 100% N1-methylpseudo-UTP and co-transcriptionally capped with an anti-reverse CAP analog (ARCA; [m₂^{7,3′-O}G(5′) ppp(5′)G]) at the 5′ end (Jena Bioscience). After in vitro transcription, the mRNAs were column-purified using MEGAclear kit (Thermo Fisher Scientific) and quantified using Nanophotometer-N60 (Implen).

TABLE 4
Primers for Template Generation

| SEQ | Primer | |
|---|---|---|
| ID NO | Direction | Sequence (5′ to 3′) |
| 17 | Forward | GCTGCAAGGCGATTAAGTTG |
| 18 | Reverse | U(2′OMe)U(2′OMe)U(2′OMe)TTTTTTTTTTTTTTTTTTTTTT |
| | | TTTTTTTTTTTTTTTTTTTTTTTTTTTTTTTTTTTTTTTTTTTTTT |
| | | TTTTTTTTTTTTTTTTTTTTTTTTTTTTTTTTTTTTTTTTTTTTTT |
| | | TTTCAGCTATGACCATGTTAATGCAG |

Using in vitro transcription, Compounds A1-A5 were generated at 50-200 μg range and were tested for IL-8 down regulation and IGF-1 expression in overexpression models of HEK-293 (Example 3) and THP-1 cells (Example 4) where IL-8 was overexpressed using respective mRNA. In addition, Compounds A6 and A7 were generated at 50-200 μg range and were tested for endogenous IL-1 beta down regulation and IGF-1 expression in THP-1 cells which were stimulated by LPS and dsDNA for endogenous secretion of IL-1 beta (Example 4). Compound A8 was generated at 50-200 μg range and was tested for endogenous TNF-α down regulation and IL-4 expression in THP-1 cells where endogenous TNF-α expression was stimulated by the treatment with LPS and R848 (Example 4). Likewise, Compound A8 was tested for TNF-α down regulation and IL-4 expression in overexpression models of HEK-293 cells where TNF-α was overexpressed using TNF-α encoding mRNA (Example 3).

Data were analyzed using GraphPad Prism 8 (San Diego, USA). For the estimation of the protein (IGF-1, IL-4, IL-8, IL-1 beta or TNF-α) levels using ELISA in the standard or the sample, the mean absorbance value of the blank was subtracted from the mean absorbance of the standards or the samples. A standard curve was generated and plotted using a four parameters nonlinear regression according to manufacturer's protocol. To determine the concentration of proteins (IGF-1, IL-4, IL-8, IL-1 beta or TNF-α) in each sample, the concentration of the protein was interpolated from the standard curve. The final protein concentration of the sample was calculated by multiplication with the dilution factor. Statistical analyses were made using a Student's t-test.

### Example 3: In Vitro Transfection of HEK-293 and IL-8 Overexpression Model in HEK-293 Cells

In Vitro Transfection of HEK-293

Human embryonic kidney cells 293 (HEK-293; ATCC CRL-1573) were maintained in Dulbecco's Modified Eagle's medium (DMEM, Biochrom) supplemented with 10% (v/v) Fetal Bovine Serum (FBS) and Penicillin-Streptomycin-Amphotericin B mixture (882087, Biozym Scientific). Cells were seeded at 20,000 cell/well in a 96 well culture plate and incubated at 37° C. in a humidified atmosphere containing 5% CO₂ for 24 hours prior to transfection. Cells were grown in DMEM growth medium containing 10% of FBS without antibiotics to reach confluency <60% before transfection. Thereafter, HEK-293 cells were transfected with specific mRNA constructs with varying concentrations (100-900 ng) using Lipofectamine 2000 (Invitrogen) following the manufacturer's instructions with the mRNA to Lipofectamine ratio of 1:1 w/v. 100 μl of DMEM was removed and replaced with 50 μl of Opti-MEM and 50 μl mRNA and Lipofectamine 2000 complex in Opti-MEM (Thermo Fisher Scientific). After 5 hours, the medium was replaced by fresh medium and the plates were incubated at 37° C. in a humidified atmosphere containing 5% CO₂ for 24 hours.

IL-8 Overexpression Model in HEK-293 Cells

To assess the simultaneous effect of IL-8 RNA interference (RNAi) and IGF-1 expression of RNA constructs (Compounds A1-A5) in HEK-293 cells, the IL-8 overexpression model was established using IL-8 mRNA transfection (300 ng/well). To assess the capability of mRNA constructs containing IL-8-targeting siRNA (Compounds A1-A5) in interfering with IL-8 expression and at the same time expressing IGF-1, the mRNA constructs (Compounds A1-A5; 300-900 ng/well) were co-transfected with IL-8 mRNA (300 ng/well). Post transfection, the cells were incubated at 37° C. in a humidified atmosphere containing 5% CO₂ for 24 hours followed by quantification of IL-8 (target gene to downregulate) and IGF-1 (Gene of Interest to overexpress) by ELISA in the cell culture supernatant.

TNF-α Overexpression Model in HEK-293 Cells

To assess the simultaneous effect of TNF-α RNA interference (RNAi) and IL-4 expression of Compound A8 in HEK-293 cells, the TNF-α overexpression model was established using TNF-α mRNA transfection (600 ng/well). To assess the capability of Compound A8 containing TNF-α targeting siRNA in TNF-α downregulation and simultaneous IL-4 expression, the cells were co-transfected with Compound A8 (600 ng/well) and TNF-α mRNA (600 ng/well). Post transfection, the cells were incubated at 37° C. in a humidified atmosphere containing 5% CO₂ for 24 hours followed by quantification of TNF-α (target gene to downregulate) and IL-4 (Gene of Interest to overexpress) by ELISA in the same cell culture supernatant.

Results

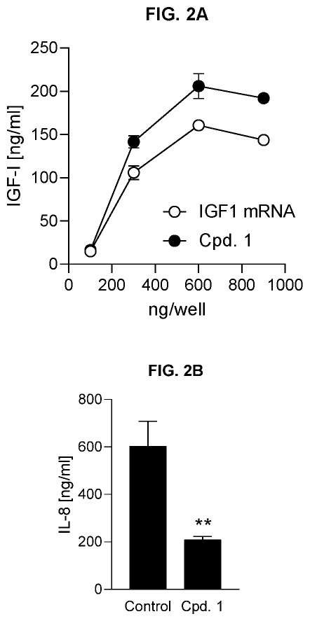

Compound A1 comprising IL-8-targeting siRNA and IGF-1 protein coding sequence was tested for IL-8 downregulation and simultaneous IGF-1 expression in HEK-293 cells (100-900 ng/well). The data demonstrate that Compound A1 expresses IGF-1 protein to the same level or above the level expressed by the control IGF-1 mRNA as shown in FIG. 2A (open circles—expression of IGF-1 from control IGF-1 mRNA; closed circles—Compound A1 IGF-1 expression). In the same experiment, the RNA interference of Compound A1 (300 ng/well) against IL-8 expression was assessed with IL-8 overexpression construct (300 ng/well) followed by IL-8 ELISA. As shown in FIG. 2B, Compound A1 (right bar) downregulated the IL-8 level compared to untreated control (left bar) (P<0.01). These assays showed that Compound A1 downregulated IL-8 by at least approximately 3-fold (65%), without reducing the expression of IGF-1.

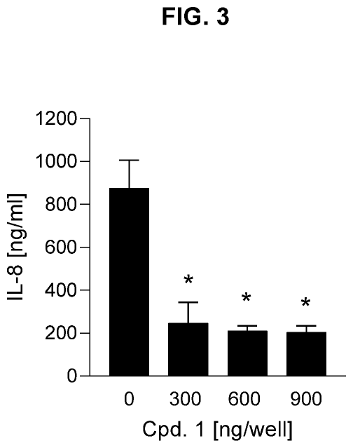

To assess the dose-dependent capability of Compound A1 in interfering with IL-8 expression in HEK-293 IL-8 overexpression model, HEK-293 cells were co-transfected with an increasing dose of Compound A1 (300-900 ng of Compound A1/well) and constant IL-8 mRNA (300 ng/well) and assessed for IL-8 expression by ELISA. As demonstrated in FIG. 3, Compound A1 mRNA constructs comprising IL-8-targeting siRNA and IGF-1 protein coding sequence inhibited IL-8 expression in HEK-293 cells in a dose-dependent manner. FIG. 3 shows that at 300 ng/well Compound A1 reduced IL-8 expression by at least approximately 3.5-fold (70%) and at 600 or 900 ng/well, Compound A1 reduced IL-8 expression by at least approximately 4.25-fold (75%).

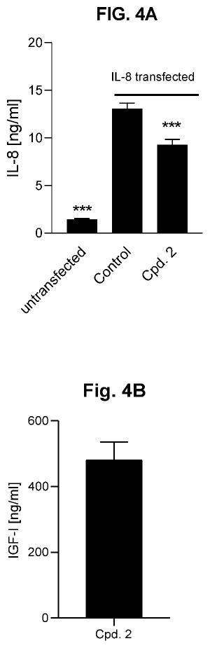

Compound A2 and Compound A3, which comprise 1× and 3×siRNA targeting IL-8, respectively, and IGF-1 protein coding sequence were tested to assess whether the presence of siRNA sequence in the same construct affect the IGF-1 expression. The HEK-293 cells were transfected with IGF-1 mRNA (600 ng/well). The results, in FIG. 4B (Compound A2) and 5B (Compound A3), show that IGF-1 is expressed from Compounds A2 and A3.

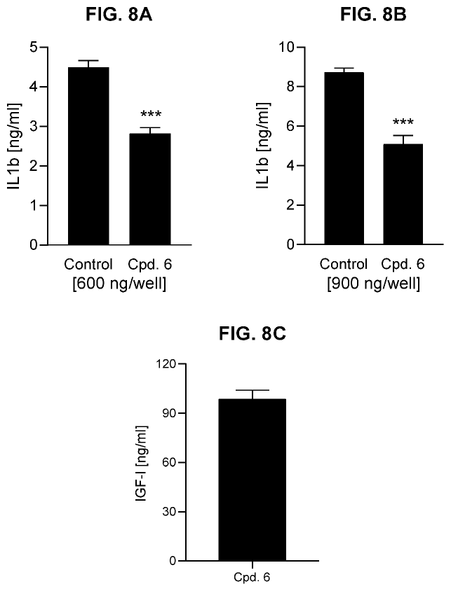

Compound A6 and Compound A7, which comprise 1× and 3×siRNA targeting IL-1 beta, respectively, and IGF-1 protein coding sequence were tested to assess whether the presence of siRNA in the same construct affect the IGF-1 expression. The HEK-293 cells were transfected with IGF-1 mRNA (600 ng/well). The results, in FIG. 8C (Compound A6) and 9C (Compound A7), show that IGF-1 is expressed from Compounds A6 and A7.

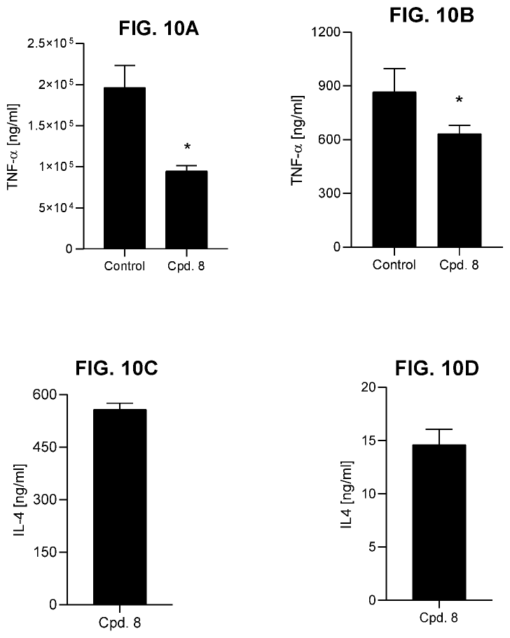

Compound A8, comprising TNF-α-targeting siRNA and IL-4 protein coding sequence was tested for TNF-α downregulation and IL-4 expression at the same time in HEK-293 cells (600 ng/well) with exogenously delivered TNF-α mRNA (600 ng/well). The data demonstrate that Compound A8 expresses IL-4 as shown in FIG. 10C. In the same experiment with the same cell culture supernatant, the RNA interference of Compound A8 (600 ng/well) against TNF-α expression from a TNF-α overexpression construct (600 ng/well) was assessed by TNF-α ELISA. As shown in FIG. 10A, Compound A8 (right bar) downregulated the TNF-α level compared to untreated control (left bar) (P<0.05). In this assay, Compound A8 downregulated TNF-α level by at least approximately 50%. These data demonstrate that Compound A8 downregulated TNF-α without affecting the IL-4 expression.

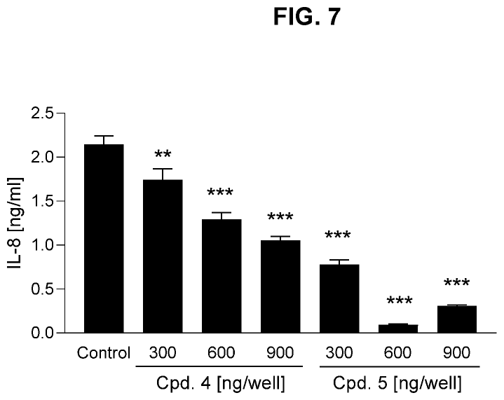

Next, Compound A4 and Compound A5, which comprise 1× and 3×siRNA targeting IL-8, respectively, but do not comprise IGF-1 coding sequence, were assessed for dose-dependent capability in interfering with IL-8 expression in HEK-293 cells. HEK-293 cells overexpressing IL-8 (600 ng of IL-8 mRNA) were transfected with various concentrations (300-900 ng/well) of Compound A4 (1×siRNA) and Compound A5 (3×siRNA). As demonstrated in FIG. 7, Compound A4 and Compound A5 inhibited IL-8 expression in HEK-293 cells in a dose-dependent manner.

### Example 4: In Vitro Transfection of THP-1 Cells, Endogenous IL-1 Beta/TNF-α Expression Model in THP-1 Cells and IL-8 Overexpression Model in THP-1 Cells

In Vitro Transfection of THP-1 Cells

Human monocyte leukemia cell line THP-1 (Sigma-Aldrich, Cat. #88081201) was maintained in growth medium (RPMI 1640 supplemented with 10% FBS and 2 mM glutamine). The cells were seeded at 30,000 THP-1 cells in a 96 well cell culture plate 72 hours before transfection and activated with 50 nM of phorbol 12-myristate 13-acetate (PMA) (Sigma-Aldrich, Cat. #P8139) diluted in growth medium. The cells were transfected with specific mRNA as mono transfection or co-transfection (300-1200 ng/well) using Lipofectamine 2000 (Thermo Fisher Scientific). 100 μl of DMEM was removed from each well and replaced with 50 μl of Opti-MEM (Thermo Fisher Scientific) and 50 μl mRNA and Lipofectamine 2000 complex in Opti-MEM. After 5 hours, the medium was replaced with fresh growth medium supplemented with 50 nM PMA and the plates were incubated at 37° C. in a humidified atmosphere containing 5% CO₂ for 24 hours.

Endogenous IL-1 Beta Expression Model in THP-1 Cells

For the endogenous secretion of IL-1 beta in THP-1 cells, THP-1 cells were stimulated with E. coli-derived lipopolysaccharide (LPS-L4391; Sigma Aldrich) at 10 μg/mL final concentration with dsDNA (a specific PCR amplicon; 50 ng/well) and incubated for 90 minutes. The induced production of IL-1 beta corresponds to the physiological conditions observed in Osteoarthritis and IVDD. Post stimulation, 50 μl of media was removed and replaced with the transfection complex containing specific mRNA constructs (Compounds A6 and A7) complexed with Lipofectamine 2000 in Opti-MEM and incubated at 37° C. in a humidified atmosphere containing 5% CO₂ for 24 hours followed by IL-1 beta quantification by ELISA.

Endogenous TNF-α Expression Model in THP-1 Cells

For the endogenous secretion of TNF-α in THP-1 cells, THP-1 cells were stimulated with E. coli-derived lipopolysaccharide (LPS-L4391; Sigma Aldrich) at 10 μg/mL final concentration with R848 (TLR7/8 agonist; Invivogen) at 1 μg/mL final concentration and incubated for 90 minutes. The induced production of TNF-α corresponds to the physiological conditions observed in psoriasis. Post stimulation, 50 μl of media was removed and replaced with the transfection complex containing specific mRNA constructs (Compound A8) complexed with Lipofectamine 2000 in Opti-MEM and incubated at 37° C. in a humidified atmosphere containing 5% CO₂ for 24 hours. Post transfection, the cell culture supernatant was collected and quantified for TNF-α (target gene to downregulate) and IL-4 (Gene of Interest to overexpress) by ELISA.

IL-8 Overexpression Model in THP-1 Cells

To assess the RNA interference (RNAi) of mRNA constructs in THP-1 cells, the IL-8 overexpression model was established using IL-8 mRNA transfection (300 ng/well). To assess the capability of mRNA constructs containing IL-8-targeting siRNA (Compounds A1-A5) in interfering with IL-8 expression, the mRNA constructs (300-900 ng/well) were co-transfected with IL-8 mRNA (300 ng/well). Post transfection, the cells were incubated at 37° C. in a humidified atmosphere containing 5% CO₂ for 24 hours followed by quantification of IL-8 and IGF-1 by ELISA.

Results

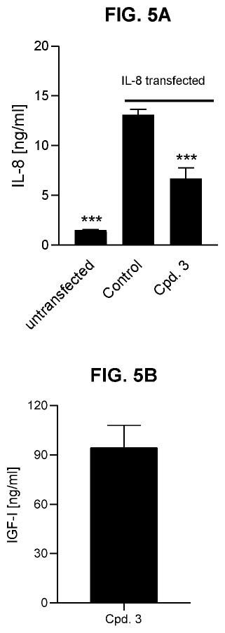

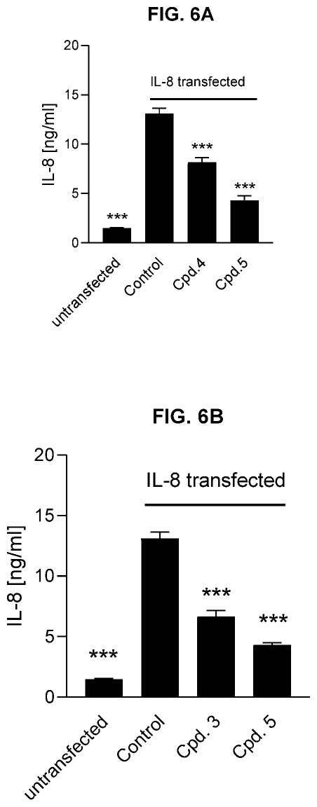

Compound A2 and Compound A3 were designed to have 1× and 3×siRNA targeting IL-8, respectively, and IGF-1 coding sequence (Tables 1 and 2) and were tested to assess whether having more than one siRNA can maximize the effect of the targeted RNAi. Compound A4 and Compound A5 were designed as internal controls, which comprise only 1× and 3×siRNA targeting IL-8, respectively, without IGF-1 coding sequence (Tables 1 and 2). As demonstrated in FIGS. 4A, 5A, 6A, and 6B, Compounds A2-A5 inhibit IL-8 expression in THP-1 cells regardless of whether the compound has IGF-1 coding sequence. Compound A2 inhibited IL-8 expression by at least approximately 30% (FIG. 4A). Compound A3 inhibited IL-8 expression by at least approximately 45% (FIG. 6B). Compound A4 inhibited IL-8 expression by approximately 40% (FIG. 6A). Compound A5 inhibited IL-8 expression by at least approximately 70% (FIGS. 6A and 6B). Therefore, the compounds having three siRNA (Compounds A3 and A5) inhibited IL-8 expression by at least approximately 45% to at least approximately 70%, whereas the compounds having one siRNA (Compounds A2 and A4) inhibited IL-8 expression by at least approximately 30% to at least approximately 40%.

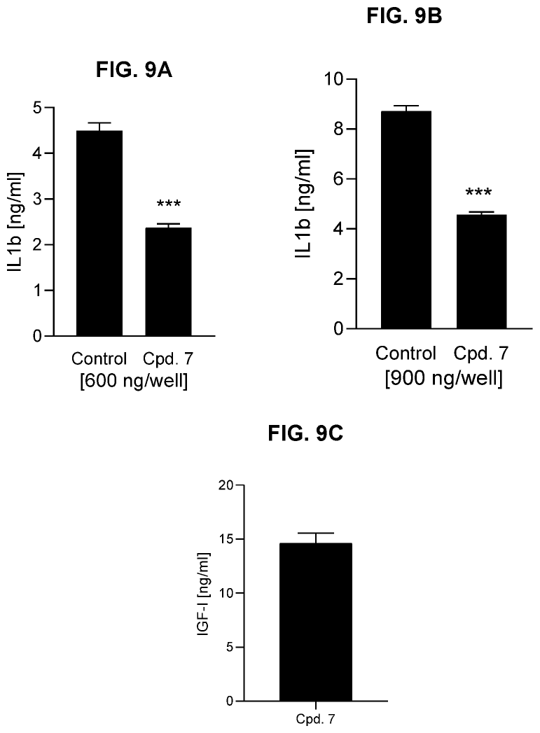

Next, the effect of Compound A6 (1×siRNA targeting IL-1 beta+IGF-1 coding sequence) and Compound A7 (3×siRNA targeting IL-1 beta+IGF-1 coding sequence) in interfering with IL-1 beta expression was evaluated in THP-1 cells stimulated with 10 μg/mL LPS and 50 ng/well dsDNA to induce endogenous IL-1 beta secretion. The established THP-1 model mimics the physiological immune condition of osteoarthritis and IVDD. As demonstrated in FIGS. 8A, 8B, 9A, and 9B, Compound A6 and Compound A7 downregulated the expression of endogenous IL-1 beta expression in THP-1 cells (P<0.001). Compound A6 downregulated IL-1 beta expression by at least approximately 40% (FIGS. 8A and 8B). Compound A7 downregulated IL-1 beta expression by at least approximately 45% to at least approximately 50% (FIGS. 9A and 9B, respectively).

The effect of Compound A8 (comprising siRNA targeting TNF-α and IL-4 coding sequence) in downregulation of TNF-α was evaluated in THP-1 cells stimulated with 10 μg/mL LPS and 1 μg/mL R848 to induce endogenous TNF-α secretion. The established THP-1 model mimics the physiological immune condition of psoriasis. As demonstrated in FIG. 10B, Compound A8 downregulated the expression of endogenous TNF-α expression in THP-1 cells (P<0.05). In this assay, Compound A8 downregulated TNF-α expression by at least approximately 20%. The same cell culture supernatant was measured for IL-4 expression and it was confirmed that IL-4 expression was not impaired (FIG. 10D).

### Example 5: Anti-Viral Construct Design, Sequence, and Synthesis

Anti-Viral Construct Design

Both siRNAs and proteins of interest are simultaneously expressed from a single transcript generated by in vitro transcription. Polynucleotide or RNA constructs are engineered to include siRNA designs as described in Cheng, et al. (2018) J. Mater. Chem. B., 6, 4638-4644, and further comprise one or more gene of interest downstream or upstream of the siRNA sequence (schematic in FIG. 1). The construct may encode or comprise more than one siRNA sequence targeting the same or different target mRNA. Likewise, the construct may comprise nucleic acid sequences of two or more genes of interest. A linker sequence may be present between any two elements of the construct (e.g., 2A peptide linker or tRNA linker).

As presented in FIG. 1, a polynucleic acid construct may comprise a T7 promoter sequence (5′ TAATACGACTCACTATA 3′; SEQ ID NO: 25) upstream of the gene of interest sequence, for RNA polymerase binding and successful in vitro transcription of both the gene of interest and siRNA in a single transcript. An alternative promoter, e.g., SP6, T3, P60, Syn5, and KP34 may be used. A transcription template is generated by PCR to produce mRNA, using primers designed to flank the T7 promoter, IFN-beta and siRNA sequences. The reverse primer includes a stretch of T(120) (SEQ ID NO: 197) to add the 120 bp length of poly(A) tail (SEQ ID NO: 193) to the mRNA.

Anti-Viral Construct Synthesis

The constructs as shown in Table 5 are synthesized by GeneArt, Germany (Thermo Fisher Scientific) as vectors containing a T7 RNA polymerase promoter (pMX, e.g., pMA-T or pMA-RQ), with codon optimization (GeneOptimizer algorithm). Table 5 shows, for each compound, the protein to be downregulated through siRNA binding to the corresponding mRNA, the number of siRNAs of the construct (e.g., either multiple siRNA targeting the same mRNA, or multiple siRNA each targeting a different mRNA), and the protein target for upregulation, i.e., the product of the gene of interest. All uridines in Compounds B1-B19 used in the examples described herein were modified to N¹-methylpseudouridine. The sequences of each construct are shown in Table 6 and annotated as indicated below the table.

TABLE 5
Summary of Compounds B1-B19

| Compound | | siRNA | #of | Protein Target | |
|---|---|---|---|---|---|
| ID | siRNA Target | Position | siRNAs | (gene of interest) | Mechanism |
| B1 | IL-6 | 3’ | 3 | IFN-β | Cytokine storm, anti- |
| | | | | | inflammation |
| B2 | IL-6 | 3’ | 1 | IFN-β | Cytokine storm, anti- |
| | | | | | inflammation |
| B3 | IL-6R | 3’ | 3 | IFN-β | Cytokine storm, anti- |
| | | | | | inflammation |
| B4 | IL-6R alpha | 3’ | 1 | IFN-β | Cytokine storm, anti- |
| | | | | | inflammation |
| B5 | IL-6R beta | 3’ | 1 | IFN-β | Cytokine storm, anti- |
| | | | | | inflammation |
| B6 | ACE2 | 3’ | 3 | IFN-β | Viral entry, anti- |
| | | | | | inflammation |
| B7 | ACE2 | 3’ | 1 | IFN-β | Viral entry, anti- |
| | | | | | inflammation |
| B8 | SARS CoV-2 | 3’ | 3 | IFN-β | Anti-viral, anti-inflammation |
| | (ORF1ab, S, N) | | | | |
| B9 | SARS CoV-2 (S) | 3’ | 1 | IFN-β | Anti-viral, anti-inflammation |
| B10 | SARS CoV-2 (N) | 3’ | 1 | IFN-β | Anti-viral, anti-inflammation |
| B11 | SARS CoV-2 (S) | 3’ | 3 | IFN-β | Anti-viral, anti-inflammation |
| B12 | SARS CoV-2 | 3’ | 3 | IFN-β | Anti-viral, anti-inflammation |
| | (ORF1ab) | | | | |
| B13 | SARS CoV-2 | 3’ | 1 | IFN-β | Anti-viral, anti-inflammation |
| | (ORF1ab) | | | | |
| B14 | SARS CoV-2 | 3’ | 1 | IFN-β | Anti-viral, anti-inflammation |
| | (ORF1ab) | | | | |
| B15 | IL6/ACE2/SARS | 3’ | 3 | IFN-β | Cytokine storm, viral entry, |
| | CoV-2 (S) | | | | anti-viral, anti-inflammation |
| B16 | IL6/ACE2/SARS | 3’ | 3 | IFN-β (1)* | Cytokine storm, viral entry, |
| | CoV-2 (S) | | | | anti-viral, anti-inflammation |
| B17 | IL6/ACE2/SARS | 3’ | 3 | IFN-β (2)* | Cytokine storm, viral entry, |
| | CoV-2 (S) | | | | anti-viral, anti-inflammation |
| B18 | SARS CoV-2 | 3’ | 3 | ACE2 soluble | Anti-viral, viral |
| | (ORF1ab, S, N) | | | receptor | neutralization |
| B19 | SARS CoV-2 (S) | 3’ | 3 | ACE2 soluble | Anti-viral, viral |
| | | | | receptor | neutralization |

*IFN-β (1) and IFN-β (2) represent the modified signal peptide (SP) to enhance secretion

TABLE 6
Sequences of Compounds B1-B19

| SEQ ID NO | Compound | Sequence (5′ to 3′) |
|---|---|---|
| 29 | Compound B1 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B1-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 93, antisense | | GGATGAACTTCGACATCCCCGAGGAAATCAAGCAGCTGCAGCA |
| 123; | | GTTCCAGAAAGAGGACGCCGCTCTGACCATCTACGAGATGCTG |
| B1-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 94, antisense | | CCACCAGATCAACCACCTGAAAACCGTGCTGGAAGAGAAGCTG |
| 124; | | GAAAAAGAGGACTTCACCCGGGGCAAGCTGATGAGCAGCCTGC |
| B1-3 sense | | ACCTGAAGCGGTACTACGGCAGAATCCTGCACTACCTGAAGGC |
| strand siRNA | | CAAAGAGTACAGCCACTGCGCCTGGACCATCGTGCGCGTGGAA |
| 95, antisense | | ATCCTGCGGAACTTCTACTTCATCAACCGGCTGACCGGCTACC |
| 125 | | TGAGAAACTGAATAGTGAGTCGTATTAACGTACCAACAAGCCC |
| | | TGAGAAAGGAGACATGTACTTG [symbol] |
| | | TTTATCTTAGAGGCATATCCCTACGTACCAACAAGAGGAGACT |
| | | TGCCTGGTGAAAACTTG [symbol] TTTAT |
| | | CTTAGAGGCATATCCCTACGTACCAACAAGAGGGCTCTTCGGC |
| | | AAATGTAACTTG [symbol] TTTATCTTAG |
| | | AGGCATATCCCTTTTATCTTAGAGGCATATCCCT |
| 30 | Compound B2 | GCCACCATGACCAACAAGTGCCTGCTGCAGATTGCCCTGCTGC |
| B2 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 94, antisense | | CTGTGGCAGCTGAACGGCCGGCTGGAATACTGCCTGAAGGACC |
| 124 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGAGG |
| | | AGACTTGCCTGGTGAAAACTTG [symbol] |
| | | TTTATCTTAGAGGCATATCCCTTTTATCTTAGAGGCATATCCC |
| | | T |
| 31 | Compound B3 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B3-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 96, antisense | | GGATGAACTTCGACATCCCCGAGGAAATCAAGCAGCTGCAGCA |
| 126; | | GTTCCAGAAAGAGGACGCCGCTCTGACCATCTACGAGATGCTG |
| B3-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 97, antisense | | CCACCAGATCAACCACCTGAAAACCGTGCTGGAAGAGAAGCTG |
| 127; | | GAAAAAGAGGACTTCACCCGGGGCAAGCTGATGAGCAGCCTGC |
| B3-3 sense | | ACCTGAAGCGGTACTACGGCAGAATCCTGCACTACCTGAAGGC |
| strand siRNA | | CAAAGAGTACAGCCACTGCGCCTGGACCATCGTGCGCGTGGAA |
| 98, antisense | | ATCCTGCGGAACTTCTACTTCATCAACCGGCTGACCGGCTACC |
| 128 | | TGAGAAACTGAATAGTGAGTCGTATTAACGTACCAACAAGTGA |
| | | GGAAGTTTCAGAACAGTACTTG [symbol] |
| | | TTTATCTTAGAGGCATATCCCTACGTACCAACAAGAACGGTCA |
| | | AAGACATTCACAACTTG [symbol] TTTAT |
| | | CTTAGAGGCATATCCCTACGTACCAACAAGGGAAGGTTACATC |
| | | AGATCATACTTG [symbol] TTTATCTTAG |
| | | AGGCATATCCCTTTTATCTTAGAGGCATATCCCT |
| 32 | Compound B4 | GCCACCATGACCAACAAGTGCCTGCTGCAGATTGCCCTGCTGC |
| B2 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 96, antisense | | CTGTGGCAGCTGAACGGCCGGCTGGAATACTGCCTGAAGGACC |
| 126 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGTGA |
| | | GGAAGTTTCAGAACAGTACTTG [symbol] |
| | | TTTATCTTAGAGGCATATCCCTTTTATCTTAGAGGCATATCCC |
| | | T |
| 33 | Compound B5 | GCCACCATGACCAACAAGTGCCTGCTGCAGATTGCCCTGCTGC |
| B5 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 98, antisense | | CTGTGGCAGCTGAACGGCCGGCTGGAATACTGCCTGAAGGACC |
| 128 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGGGA |
| | | AGGTTACATCAGATCATACTTG [symbol] |
| | | TTTATCTTAGAGGCATATCCCTTTTATCTTAGAGGCATATCCC |
| | | T |
| 34 | Compound B6 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B6-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 99, antisense | | GGATGAACTTCGACATCCCCGAGGAAATCAAGCAGCTGCAGCA |
| 129; | | GTTCCAGAAAGAGGACGCCGCTCTGACCATCTACGAGATGCTG |
| B6-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 100, | | CCACCAGATCAACCACCTGAAAACCGTGCTGGAAGAGAAGCTG |
| antisense | | GAAAAAGAGGACTTCACCCGGGGCAAGCTGATGAGCAGCCTGC |
| 130; | | ACCTGAAGCGGTACTACGGCAGAATCCTGCACTACCTGAAGGC |
| B6-3 sense | | CAAAGAGTACAGCCACTGCGCCTGGACCATCGTGCGCGTGGAA |
| strand siRNA | | ATCCTGCGGAACTTCTACTTCATCAACCGGCTGACCGGCTACC |
| 101, | | TGAGAAACTGAATAGTGAGTCGTATTAACGTACCAACAAGCAG |
| antisense 131 | | CTGAGGCCATTATATGAACTTG [symbol] |
| | | TTTATCTTAGAGGCATATCCCTACGTACCAACAAGGACCCAGG |
| | | AAATGTTCAGAAACTTG [symbol] TTTAT |
| | | CTTAGAGGCATATCCCTACGTACCAACAAGGCTGAAAGACCAG |
| | | AACAAGAACTTG [symbol] TTTATCTTAG |
| | | AGGCATATCCCTTTTATCTTAGAGGCATATCCCT |
| 35 | Compound B7 | GCCACCATGACCAACAAGTGCCTGCTGCAGATTGCCCTGCTGC |
| B6 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 99, antisense | | CTGTGGCAGCTGAACGGCCGGCTGGAATACTGCCTGAAGGACC |
| 129 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGCAG |
| | | CTGAGGCCATTATATGAACTTG [symbol] |
| | | TTTATCTTAGAGGCATATCCCTTTTATCTTAGAGGCATATCCC |
| | | T |
| 36 | Compound B8 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B8-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 102, | | GGATGAACTTCGACATCCCCGAGGAAATCAAGCAGCTGCAGCA |
| antisense | | GTTCCAGAAAGAGGACGCCGCTCTGACCATCTACGAGATGCTG |
| 132; | | CAGAACATCTTCGCCATCTTCCGGCAGGACAGCAGCTCCACAG |
| B8-2 sense | | GCTGGAACGAGACAATCGTGGAAAATCTGCTGGCCAACGTGTA |
| strand siRNA | | CCACCAGATCAACCACCTGAAAACCGTGCTGGAAGAGAAGCTG |
| 107, | | GAAAAAGAGGACTTCACCCGGGGCAAGCTGATGAGCAGCCTGC |
| antisense | | ACCTGAAGCGGTACTACGGCAGAATCCTGCACTACCTGAAGGC |
| 137; | | CAAAGAGTACAGCCACTGCGCCTGGACCATCGTGCGCGTGGAA |
| B8-3 sense | | ATCCTGCGGAACTTCTACTTCATCAACCGGCTGACCGGCTACC |
| strand siRNA | | TGAGAAACTGAATAGTGAGTCGTATTAACGTACCAACAAGTGT |
| 109, | | GACCGAAAGGTAAGATGACTTG [symbol] |
| antisense 139 | | TTTATCTTAGAGGCATATCCCTACGTACCAACAAGAGGTGATG |
| | | AAGTCAGACAAAACTTG [symbol] TTTAT |
| | | CTTAGAGGCATATCCCTACGTACCAACAAGCAACTGAGGGAGC |
| | | CTTGAATACTTG [symbol] TTTATCTTAG |
| | | AGGCATATCCCTTTTATCTTAGAGGCATATCCCT |
| 37 | Compound B9 | GCCACCATGACCAACAAGTGCCTGCTGCAGATTGCCCTGCTGC |
| B9 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 107, | | CTGTGGCAGCTGAACGGCCGGCTGGAATACTGCCTGAAGGACC |
| antisense 137 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGAGG |
| | | TGATGAAGTCAGACAAAACTTGT [symbol] |
| | | TTTATCTTAGAGGCATATCCCTTTTATCTTAGAGGCATATCCC |
| | | T |
| 38 | Compound B10 | GCCACCATGACCAACAAGTGCCTGCTGCAGATTGCCCTGCTGC |
| B10 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 109, | | CTGTGGCAGCTGAACGGCCGGCTGGAATACTGCCTGAAGGACC |
| antisense 139 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGCAA |
| | | CTGAGGGAGCCTTGAATACTTG [symbol] |
| | | TTTATCTTAGAGGCATATCCCTTTTATCTTAGAGGCATATCCC |
| | | T |
| 39 | Compound B11 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B11-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 106, | | GGATGAACTTCGACATCCCCGAGGAAATCAAGCAGCTGCAGCA |
| antisense | | GTTCCAGAAAGAGGACGCCGCTCTGACCATCTACGAGATGCTG |
| 136; | | CAGAACATCTTCGCCATCTTCCGGCAGGACAGCAGCTCCACAG |
| B11-2 sense | | GCTGGAACGAGACAATCGTGGAAAATCTGCTGGCCAACGTGTA |
| strand siRNA | | CCACCAGATCAACCACCTGAAAACCGTGCTGGAAGAGAAGCTG |
| 107, | | GAAAAAGAGGACTTCACCCGGGGCAAGCTGATGAGCAGCCTGC |
| antisense | | ACCTGAAGCGGTACTACGGCAGAATCCTGCACTACCTGAAGGC |
| 137; | | CAAAGAGTACAGCCACTGCGCCTGGACCATCGTGCGCGTGGAA |
| B11-3 sense | | ATCCTGCGGAACTTCTACTTCATCAACCGGCTGACCGGCTACC |
| strand siRNA | | TGAGAAACTGAATAGTGAGTCGTATTAACGTACCAACAAGTTG |
| 108, | | CTGATTATTCTGTCCTAACTTG [symbol] |
| antisense 138 | | TTTATCTTAGAGGCATATCCCTACGTACCAACAAGAGGTGATG |
| | | AAGTCAGACAAAACTTG [symbol] TTTAT |
| | | CTTAGAGGCATATCCCTACGTACCAACAAGCCGGTAGCACACC |
| | | TTGTAATACTTG [symbol] TTTATCTTAG |
| | | AGGCATATCCCTTTTATCTTAGAGGCATATCCCT |
| 40 | Compound B12 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B12-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 103, | | GGATGAACTTCGACATCCCCGAGGAAATCAAGCAGCTGCAGCA |
| antisense | | GTTCCAGAAAGAGGACGCCGCTCTGACCATCTACGAGATGCTG |
| 133; | | CAGAACATCTTCGCCATCTTCCGGCAGGACAGCAGCTCCACAG |
| B12-2 sense | | GCTGGAACGAGACAATCGTGGAAAATCTGCTGGCCAACGTGTA |
| strand siRNA | | CCACCAGATCAACCACCTGAAAACCGTGCTGGAAGAGAAGCTG |
| 104, | | GAAAAAGAGGACTTCACCCGGGGCAAGCTGATGAGCAGCCTGC |
| antisense | | ACCTGAAGCGGTACTACGGCAGAATCCTGCACTACCTGAAGGC |
| 134; | | CAAAGAGTACAGCCACTGCGCCTGGACCATCGTGCGCGTGGAA |
| B12-3 sense | | ATCCTGCGGAACTTCTACTTCATCAACCGGCTGACCGGCTACC |
| strand siRNA | | TGAGAAACTGAATAGTGAGTCGTATTAACGTACCAACAATTTA |
| 105, | | AATATTGGGATCAGACACTTG [symbol] TT |
| antisense 135 | | TATCTTAGAGGCATATCCCTACGTACCAACAAAAGAATAGAGC |
| | | TCGCACACTTG [symbol] TTTATCTTAGAGGCA |
| | | TATCCCTACGTACCAACAAACTGTTGATTCATCACAGGGACTT |
| | | GCCC [symbol] TTTATCTTAGAGGCATATCCCT |
| | | TTTATCTTAGAGGCATATCCCT |
| 41 | Compound B13 | GCCACCATGACCAACAAGTGCCTGCTGCAGATTGCCCTGCTGC |
| B13 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 104, | | CTGTGGCAGCTGAACGGCCGGCTGGAATACTGCCTGAAGGACC |
| antisense 134 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAAAGA |
| | | ATAGAGCTCGCACACTTG [symbol] TTTATCTT |
| | | AGAGGCATATCCCTTTTATCTTAGAGGCATATCCCT |
| 42 | Compound B14 | GCCACCATGACCAACAAGTGCCTGCTGCAGATTGCCCTGCTGC |
| B14 sense | | TGTGCTTCAGCACAACAGCCCTGAGCATGAGCTACAACCTGCT |
| strand siRNA | | GGGCTTCCTGCAGCGGAGCAGCAACTTCCAGTGCCAGAAACTG |
| 102, | | CTGTGGCAGCTGAACGGCCGGCTGGAATACTGCCTGAAGGACC |
| antisense 132 | | GGATGAACTTCGACATCCCCGAGGAAATCAAGCAGCTGCAGCA |
| | | GTTCCAGAAAGAGGACGCCGCTCTGACCATCTACGAGATGCTG |
| | | CAGAACATCTTCGCCATCTTCCGGCAGGACAGCAGCTCCACAG |
| | | GCTGGAACGAGACAATCGTGGAAAATCTGCTGGCCAACGTGTA |
| | | CCACCAGATCAACCACCTGAAAACCGTGCTGGAAGAGAAGCTG |
| | | GAAAAAGAGGACTTCACCCGGGGCAAGCTGATGAGCAGCCTGC |
| | | ACCTGAAGCGGTACTACGGCAGAATCCTGCACTACCTGAAGGC |
| | | CAAAGAGTACAGCCACTGCGCCTGGACCATCGTGCGCGTGGAA |
| | | ATCCTGCGGAACTTCTACTTCATCAACCGGCTGACCGGCTACC |
| | | TGAGAAACTGAATAGTGAGTCGTATTAACGTACCAACAAGTGT |
| | | GACCGAAAGGTAAGATGACTTG [symbol] |
| | | TTTATCTTAGAGGCATATCCCTTTTATCTTAGAGGCATATCCC |
| | | T |
| 43 | Compound B15 | GCCACCATGACCAACAAGTGCCTGCTGCAGATTGCCCTGCTGC |
| 5′ to 3′: | | TGTGCTTCAGCACAACAGCCCTGAGCATGAGCTACAACCTGCT |
| B15-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 94, antisense | | GGATGAACTTCGACATCCCCGAGGAAATCAAGCAGCTGCAGCA |
| 124; | | GTTCCAGAAAGAGGACGCCGCTCTGACCATCTACGAGATGCTG |
| B15-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 99, antisense | | CCACCAGATCAACCACCTGAAAACCGTGCTGGAAGAGAAGCTG |
| 129; | | GAAAAAGAGGACTTCACCCGGGGCAAGCTGATGAGCAGCCTGC |
| B15-3 sense | | ACCTGAAGCGGTACTACGGCAGAATCCTGCACTACCTGAAGGC |
| strand siRNA | | CAAAGAGTACAGCCACTGCGCCTGGACCATCGTGCGCGTGGAA |
| 109, | | ATCCTGCGGAACTTCTACTTCATCAACCGGCTGACCGGCTACC |
| antisense 139 | | TGAGAAACTGAATAGTGAGTCGTATTAACGTACCAACAAGAGG |
| | | AGACTTGCCTGGTGAAAACTTG [symbol] |
| | | TTTATCTTAGAGGCATATCCCTACGTACCAACAAGCAGCTGAG |
| | | GCCATTATATGAACTTG [symbol] TTTAT |
| | | CTTAGAGGCATATCCCTACGTACCAACAAGCAACTGAGGGAGC |
| | | CTTGAATACTTG [symbol] TTTATCTTAG |
| | | AGGCATATCCCTTTTATCTTAGAGGCATATCCCT |
| 44 | Compound | GCCACCATGCTCCTGATCTGCCTGCTGGTGATTGCCCTGCTGC |
| 5′ to 3′: | B16* | TGTGCTTCAGCACAACAGCCCTGAGCATGAGCTACAACCTGCT |
| B16-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 94, antisense | | GGATGAACTTCGACATCCCCGAGGAAATCAAGCAGCTGCAGCA |
| 124; | | GTTCCAGAAAGAGGACGCCGCTCTGACCATCTACGAGATGCTG |
| B16-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 99, antisense | | CCACCAGATCAACCACCTGAAAACCGTGCTGGAAGAGAAGCTG |
| 129; | | GAAAAAGAGGACTTCACCCGGGGCAAGCTGATGAGCAGCCTGC |
| B16-3 sense | | ACCTGAAGCGGTACTACGGCAGAATCCTGCACTACCTGAAGGC |
| strand siRNA | | CAAAGAGTACAGCCACTGCGCCTGGACCATCGTGCGCGTGGAA |
| 109, | | ATCCTGCGGAACTTCTACTTCATCAACCGGCTGACCGGCTACC |
| antisense 139 | | TGAGAAACTGAATAGTGAGTCGTATTAACGTACCAACAAGAGG |
| | | AGACTTGCCTGGTGAAAACTTG [symbol] |
| | | TTTATCTTAGAGGCATATCCCTACGTACCAACAAGCAGCTGAG |
| | | GCCATTATATGAACTTG [symbol] TTTAT |
| | | CTTAGAGGCATATCCCTACGTACCAACAAGCAACTGAGGGAGC |
| | | CTTGAATACTTG [symbol] TTTATCTTAG |
| | | AGGCATATCCCTTTTATCTTAGAGGCATATCCCT |
| 45 | Compound | GCCACCATGCTCCTGAAGCTCCTGCTGGTGATTGCCCTGCTGG |
| 5′ to 3′: | B17* | CCTGCTTCAGCACAACAGCCCTGAGCATGAGCTACAACCTGCT |
| B17-1 sense | | GGGCTTCCTGCAGCGGAGCAGCAACTTCCAGTGCCAGAAACTG |
| strand siRNA | | CTGTGGCAGCTGAACGGCCGGCTGGAATACTGCCTGAAGGACC |
| 94, antisense | | GGATGAACTTCGACATCCCCGAGGAAATCAAGCAGCTGCAGCA |
| 124; | | GTTCCAGAAAGAGGACGCCGCTCTGACCATCTACGAGATGCTG |
| B17-2 sense | | CAGAACATCTTCGCCATCTTCCGGCAGGACAGCAGCTCCACAG |
| strand siRNA | | GCTGGAACGAGACAATCGTGGAAAATCTGCTGGCCAACGTGTA |
| 99, antisense | | CCACCAGATCAACCACCTGAAAACCGTGCTGGAAGAGAAGCTG |
| 129; | | GAAAAAGAGGACTTCACCCGGGGCAAGCTGATGAGCAGCCTGC |
| B17-3 sense | | ACCTGAAGCGGTACTACGGCAGAATCCTGCACTACCTGAAGGC |
| strand siRNA | | CAAAGAGTACAGCCACTGCGCCTGGACCATCGTGCGCGTGGAA |
| 109, | | ATCCTGCGGAACTTCTACTTCATCAACCGGCTGACCGGCTACC |
| antisense 139 | | TGAGAAACTGAATAGTGAGTCGTATTAACGTACCAACAAGAGG |
| | | AGACTTGCCTGGTGAAAACTTG [symbol] |
| | | TTTATCTTAGAGGCATATCCCTACGTACCAACAAGCAGCTGAG |
| | | GCCATTATATGAACTTG [symbol] TTTAT |
| | | CTTAGAGGCATATCCCTACGTACCAACAAGCAACTGAGGGAGC |
| | | CTTGAATACTTG [symbol] TTTATCTTAG |
| | | AGGCATATCCCTTTTATCTTAGAGGCATATCCCT |
| 46 | Compound B18 | GCCACCATGTCTAGCAGCTCTTGGCTGCTGCTGTCTCTGGTGG |
| 5′ to 3′: | | CTGTGACAGCCGCTCAGAGCACCATTGAGGAACAGGCCAAGAC |
| B18-1 sense | | CTTCCTGGACAAGTTCAACCACGAGGCCGAGGACCTGTTCTAC |
| strand siRNA | | CAGTCTAGCCTGGCCAGCTGGAACTACAACACCAACATCACCG |
| 102, | | AAGAGAACGTGCAGAACATGAACAACGCCGGCGACAAGTGGAG |
| antisense | | CGCCTTCCTGAAAGAGCAGAGCACACTGGCCCAGATGTACCCT |
| 132; | | CTGCAAGAGATCCAGAACCTGACCGTGAAGCTCCAGCTGCAGG |
| B18-2 sense | | CCCTCCAGCAGAATGGAAGCTCTGTGCTGAGCGAGGACAAGAG |
| strand siRNA | | CAAGCGGCTGAACACCATCCTGAATACCATGAGCACCATCTAC |
| 107, | | AGCACCGGCAAAGTGTGCAACCCCGACAATCCCCAAGAGTGCC |
| antisense | | TGCTGCTGGAACCCGGCCTGAATGAGATCATGGCCAACAGCCT |
| 137; | | GGACTACAACGAGAGACTGTGGGCCTGGGAGTCTTGGAGAAGC |
| B18-3 sense | | GAAGTGGGAAAGCAGCTGCGGCCCCTGTACGAGGAATACGTGG |
| strand siRNA | | TGCTGAAGAACGAGATGGCCAGAGCCAACCACTACGAGGACTA |
| 109, | | CGGCGACTATTGGAGAGGCGACTACGAAGTGAATGGCGTGGAC |
| antisense 139 | | GGCTACGACTACAGCAGAGGCCAGCTGATCGAGGACGTGGAAC |
| | | ACACCTTCGAGGAAATCAAGCCTCTGTACGAGCATCTGCACGC |
| | | CTACGTGCGGGCCAAGCTGATGAATGCTTACCCCAGCTACATC |
| | | AGCCCCATCGGCTGTCTGCCTGCTCATCTGCTGGGAGACATGT |
| | | GGGGCAGATTCTGGACCAACCTGTACAGCCTGACAGTGCCCTT |
| | | CGGCCAGAAACCTAACATCGACGTGACCGACGCCATGGTGGAT |
| | | CAGGCTTGGGATGCCCAGCGGATCTTCAAAGAGGCCGAGAAGT |
| | | TCTTCGTGTCCGTGGGCCTGCCTAATATGACCCAAGGCTTCTG |
| | | GGAGAACTCCATGCTGACAGACCCCGGCAATGTGCAGAAAGCC |
| | | GTGTGTCATCCTACCGCCTGGGATCTCGGCAAGGGCGACTTCA |
| | | GAATCCTGATGTGCACCAAAGTGACGATGGACGACTTCCTGAC |
| | | AGCCCACCACGAGATGGGCCACATCCAGTACGATATGGCCTAC |
| | | GCCGCTCAGCCCTTCCTGCTGAGAAATGGCGCCAATGAGGGCT |
| | | TCCACGAAGCCGTGGGAGAGATCATGAGCCTGTCTGCCGCCAC |
| | | ACCTAAGCACCTGAAGTCTATCGGACTGCTGAGCCCCGACTTC |
| | | CAAGAGGACAACGAGACAGAGATCAACTTCCTGCTCAAGCAGG |
| | | CCCTGACCATCGTGGGCACACTGCCCTTTACCTACATGCTGGA |
| | | AAAGTGGCGGTGGATGGTCTTTAAGGGCGAGATCCCCAAGGAC |
| | | CAGTGGATGAAGAAATGGTGGGAGATGAAGCGCGAGATCGTGG |
| | | GCGTTGTGGAACCTGTGCCTCACGACGAGACATACTGCGATCC |
| | | TGCCAGCCTGTTTCACGTGTCCAACGACTACTCCTTCATCCGG |
| | | TACTACACCCGGACACTGTACCAGTTCCAGTTTCAAGAGGCTC |
| | | TGTGCCAGGCCGCCAAGCACGAAGGACCTCTGCACAAGTGCGA |
| | | CATCAGCAACTCTACAGAGGCCGGACAGAAACTGTTCAACATG |
| | | CTGCGGCTGGGCAAGAGCGAGCCTTGGACACTGGCTCTGGAAA |
| | | ATGTCGTGGGCGCCAAGAATATGAACGTGCGGCCACTGCTGAA |
| | | CTACTTCGAGCCCCTGTTCACCTGGCTGAAGGACCAGAACAAG |
| | | AACAGCTTCGTCGGCTGGTCCACCGATTGGAGCCCTTACGCCG |
| | | ACCAGAGCATCAAAGTGCGGATCAGCCTGAAAAGCGCCCTGGG |
| | | CGATAAGGCCTATGAGTGGAACGACAATGAGATGTACCTGTTC |
| | | CGGTCCAGCGTGGCCTATGCTATGCGGCAGTACTTTCTGAAAG |
| | | TCAAGAACCAGATGATCCTGTTCGGCGAAGAGGATGTGCGCGT |
| | | GGCCAACCTGAAGCCTCGGATCAGCTTCAACTTCTTCGTGACT |
| | | GCCCCTAAGAACGTGTCCGACATCATCCCCAGAACCGAGGTGG |
| | | AAAAGGCCATCAGAATGAGCAGAAGCCGGATCAACGACGCCTT |
| | | CCGGCTGAACGACAACTCCCTGGAATTCCTGGGCATTCAGCCC |
| | | ACACTGGGCCCTCCAAATCAGCCTCCTGTGTCCTAAATAGTGA |
| | | GTCGTATTAACGTACCAACAAGTGTGACCGAAAGGTAAGATGA |
| | | CTTG [symbol] TTTATCTTAGAGGCATAT |
| | | CCCTACGTACCAACAAGAGGTGATGAAGTCAGACAAAACTTG [symbol] |
| | | [symbol] TTTATCTTAGAGGCATATCCCTA |
| | | CGTACCAACAAGCAACTGAGGGAGCCTTGAATACTTG [symbol] |
| | | [symbol] TTTATCTTAGAGGCATATCCCTTTTATC |
| | | TTAGAGGCATATCCCT |
| 47 | Compound B19 | GCCACCATGTCTAGCAGCTCTTGGCTGCTGCTGTCTCTGGTGG |
| 5′ to 3′: | | CTGTGACAGCCGCTCAGAGCACCATTGAGGAACAGGCCAAGAC |
| B19-1 sense | | CTTCCTGGACAAGTTCAACCACGAGGCCGAGGACCTGTTCTAC |
| strand siRNA | | CAGTCTAGCCTGGCCAGCTGGAACTACAACACCAACATCACCG |
| 106, | | AAGAGAACGTGCAGAACATGAACAACGCCGGCGACAAGTGGAG |
| antisense | | CGCCTTCCTGAAAGAGCAGAGCACACTGGCCCAGATGTACCCT |
| 136; | | CTGCAAGAGATCCAGAACCTGACCGTGAAGCTCCAGCTGCAGG |
| B19-2 sense | | CCCTCCAGCAGAATGGAAGCTCTGTGCTGAGCGAGGACAAGAG |
| strand siRNA | | CAAGCGGCTGAACACCATCCTGAATACCATGAGCACCATCTAC |
| 107, | | AGCACCGGCAAAGTGTGCAACCCCGACAATCCCCAAGAGTGCC |
| antisense | | TGCTGCTGGAACCCGGCCTGAATGAGATCATGGCCAACAGCCT |
| 137; | | GGACTACAACGAGAGACTGTGGGCCTGGGAGTCTTGGAGAAGC |
| B19-3 sense | | GAAGTGGGAAAGCAGCTGCGGCCCCTGTACGAGGAATACGTGG |
| strand siRNA | | TGCTGAAGAACGAGATGGCCAGAGCCAACCACTACGAGGACTA |
| 108, | | CGGCGACTATTGGAGAGGCGACTACGAAGTGAATGGCGTGGAC |
| antisense 138 | | GGCTACGACTACAGCAGAGGCCAGCTGATCGAGGACGTGGAAC |
| | | ACACCTTCGAGGAAATCAAGCCTCTGTACGAGCATCTGCACGC |
| | | CTACGTGCGGGCCAAGCTGATGAATGCTTACCCCAGCTACATC |
| | | AGCCCCATCGGCTGTCTGCCTGCTCATCTGCTGGGAGACATGT |
| | | GGGGCAGATTCTGGACCAACCTGTACAGCCTGACAGTGCCCTT |
| | | CGGCCAGAAACCTAACATCGACGTGACCGACGCCATGGTGGAT |
| | | CAGGCTTGGGATGCCCAGCGGATCTTCAAAGAGGCCGAGAAGT |
| | | TCTTCGTGTCCGTGGGCCTGCCTAATATGACCCAAGGCTTCTG |
| | | GGAGAACTCCATGCTGACAGACCCCGGCAATGTGCAGAAAGCC |
| | | GTGTGTCATCCTACCGCCTGGGATCTCGGCAAGGGCGACTTCA |
| | | GAATCCTGATGTGCACCAAAGTGACGATGGACGACTTCCTGAC |
| | | AGCCCACCACGAGATGGGCCACATCCAGTACGATATGGCCTAC |
| | | GCCGCTCAGCCCTTCCTGCTGAGAAATGGCGCCAATGAGGGCT |
| | | TCCACGAAGCCGTGGGAGAGATCATGAGCCTGTCTGCCGCCAC |
| | | ACCTAAGCACCTGAAGTCTATCGGACTGCTGAGCCCCGACTTC |
| | | CAAGAGGACAACGAGACAGAGATCAACTTCCTGCTCAAGCAGG |
| | | CCCTGACCATCGTGGGCACACTGCCCTTTACCTACATGCTGGA |
| | | AAAGTGGCGGTGGATGGTCTTTAAGGGCGAGATCCCCAAGGAC |
| | | CAGTGGATGAAGAAATGGTGGGAGATGAAGCGCGAGATCGTGG |
| | | GCGTTGTGGAACCTGTGCCTCACGACGAGACATACTGCGATCC |
| | | TGCCAGCCTGTTTCACGTGTCCAACGACTACTCCTTCATCCGG |
| | | TACTACACCCGGACACTGTACCAGTTCCAGTTTCAAGAGGCTC |
| | | TGTGCCAGGCCGCCAAGCACGAAGGACCTCTGCACAAGTGCGA |
| | | CATCAGCAACTCTACAGAGGCCGGACAGAAACTGTTCAACATG |
| | | CTGCGGCTGGGCAAGAGCGAGCCTTGGACACTGGCTCTGGAAA |
| | | ATGTCGTGGGCGCCAAGAATATGAACGTGCGGCCACTGCTGAA |
| | | CTACTTCGAGCCCCTGTTCACCTGGCTGAAGGACCAGAACAAG |
| | | AACAGCTTCGTCGGCTGGTCCACCGATTGGAGCCCTTACGCCG |
| | | ACCAGAGCATCAAAGTGCGGATCAGCCTGAAAAGCGCCCTGGG |
| | | CGATAAGGCCTATGAGTGGAACGACAATGAGATGTACCTGTTC |
| | | CGGTCCAGCGTGGCCTATGCTATGCGGCAGTACTTTCTGAAAG |
| | | TCAAGAACCAGATGATCCTGTTCGGCGAAGAGGATGTGCGCGT |
| | | GGCCAACCTGAAGCCTCGGATCAGCTTCAACTTCTTCGTGACT |
| | | GCCCCTAAGAACGTGTCCGACATCATCCCCAGAACCGAGGTGG |
| | | AAAAGGCCATGAGAATGAGCAGAAGCCGGATCAAGGACGCCTT |
| | | CCGGCTGAACGACAACTCCCTGGAATTCCTGGGCATTCAGCCC |
| | | ACACTGGGCCCTCCAAATCAGCCTCCTGTGTCCTAAATAGTGA |
| | | GTCGTATTAACGTACCAACAAGTTGCTGATTATTCTGTCCTAA |
| | | CTTG [symbol] TTTATCTTAGAGGCATAT |
| | | CCCTACGTACCAACAAGAGGTGATGAAGTCAGACAAAACTTG [symbol] |
| | | [symbol] TTTATCTTAGAGGCATATCCCTA |
| | | CGTACCAACAAGCCGGTAGCACACCTTGTAATACTTG [symbol] |
| | | [symbol] TTTATCTTAGAGGCATATCCCTTTTATC |
| | | TTAGAGGCATATCCCT |

Bold = Sense siRNA strand
Bold and Italics = Anti-sense siRNA strand
Underline = Signal peptide
Italics = Kozak sequence
*Bolding within the underlined sequence indicates the modified IFN-β signal peptide.

### Example 6: In Vitro Transcription of Anti-Viral RNA Constructs and Data Analysis

PCR-based in vitro transcription is carried out using the pMX vectors encoding Compounds B1-B19 to produce mRNA. A transcription template is generated by PCR using the forward and reverse primers in Table 4. The poly(A) tail is encoded in the template resulting in a 120 bp poly(A) tail (SEQ ID NO: 193). Optimizations are made as needed due to achieve specific amplification given the repetitive sequences of siRNA flanking regions. Optimizations include: 1) decreasing the amount of vector DNA, 2) changing the DNA polymerase (Q5 hot start polymerase, New England Biolabs), 3) reducing denaturation time (30 seconds to 10 seconds) and extension time (45 seconds/kb to 10 seconds/kb) for each cycle of PCR, 4) increasing the annealing time (10 seconds to 30 seconds) for each cycle of PCR, and 5) increasing the final extension time (up to 15 minutes) for each cycle of PCR. In addition, to avoid non-specific primer binding, the PCR reaction mixture is prepared on ice, including thawing reagents, and the number of PCR cycles is reduced to 25.

For in vitro transcription, T7 RNA polymerase (MEGAscript kit, Thermo Fisher Scientific) is used at 37° C. for 2 hours. Synthesized RNAs are chemically modified with 100% N1-methylpseudo-UTP and co-transcriptionally capped with an anti-reverse CAP analog (ARCA; [m₂^{7,3′-O}G(5)ppp(5′)G]) at the 5′ end (Jena Bioscience). After in vitro transcription, the mRNAs are column-purified using MEGAclear kit (Thermo Fisher Scientific) and quantified using Nanophotometer-N60 (Implen).

Using in vitro transcription, Compounds B1-B17 are generated and tested for target mRNA/protein down regulation and gene of interest/protein of interest expression and compared with overexpression models wherein the gene of interest/protein of interest is overexpressed.

Data are analyzed using GraphPad Prism 8 (San Diego, USA). For the estimation of protein levels using ELISA in the standard or the sample, the mean absorbance value of the blank is subtracted from the mean absorbance of the standards or the samples. A standard curve is generated and plotted using a four parameters nonlinear regression according to manufacturer's protocol. To determine the concentration of a protein in each sample, the concentration of each protein is interpolated from the standard curve. The final protein concentration of the sample is calculated by multiplication with the dilution factor. Statistical analyses are carried out using a Student's t-test. The percent of GFP positive cells is calculated using SoftMax Pro tool. Relative quantification of viral RNA by qPCR are analyzed by pair-wise fixed reallocation randomization tests with REST 2009 software.

### Example 7: A549 Cell IFN-Beta Overexpression Model

In Vitro Transfection of A549 Cells with IFN-Beta Overexpression Compounds

A549 cells are typical alveolar type II (ATII) cells derived from human lung carcinoma. Since COVID-19 mortality primarily is associated with respiratory illness due to the high viral entry receptor (ACE2) expression in host ATII cells, A549 cells are used to mimic the clinical situation. The A549 cells (Sigma-Aldrich, Buchs Switzerland Cat. #6012804) will be maintained on Dulbecco's Modified Eagle's medium-high glucose (DMEM, Sigma-Aldrich, Buchs Switzerland cat #D0822) supplemented with 10% FBS (Thermofischer, Basel, Switzerland cat #10500-064). To assess the IFN-beta expression the A549 cells are plated at a density of 10,000 cells/well in a regular growth medium 24 hours prior to transfection. Thereafter, cells are transfected with Compounds B1-19 (0.3-0.6 micrograms) using Lipofectamine 2000 (www.invitrogen.com) following the manufacturer's instructions. 100 μl of DMEM are removed and 50 μl of Opti-MEM (www.thermofisher.com) are added to each well followed by 50 μl mRNA and Lipofectamine 2000 complex in Opti-MEM. After 5 hours of incubation, the medium is replaced by fresh growth medium and the plates are incubated for 24 hours at 37° C. in a humidified atmosphere containing 5% CO₂ followed by IFN-beta quantification by ELISA (Human IFN-beta bioluminescent ELISA kit 2.0, Cat. Code: luex-hifnbv2, Invivogen).

### Example 8: Endogenous IL-6 Stimulation Model in A549 Cells

In Vitro Transfection of A549 Cells with IL-6 Suppressing Compounds

For the endogenous secretion of IL-6 in A549 cells, A549 cells are stimulated with recombinant human IL1-beta (20 ng/mL; Cat. Code: rcyec-hil1b; Invivogen) and recombinant human TNF-alpha (20 ng/mL; Cat. Code: rcyc-htnfa; Invivogen) and incubated for 120 minutes. The induced production of IL-6 corresponds to the physiological conditions observed in COVID-19. Post stimulation, 50 μl of media are removed and replaced with the transfection complex containing specific mRNA constructs (Compounds B1, B2, B15, B16 and B17) complexed with Lipofectamine 2000 in Opti-MEM and incubated at 37° C. in a humidified atmosphere containing 5% CO₂ for 24 hours followed by IL-6 quantification by ELISA (ThermoFisher Scientific, cat #88-7066-22). A reduction in IL-6 compared to untreated samples is confirmed. To verify the functional suppression of IL-6, HEK-Blue™ IL-6 reporter cells stably transfected with IL-6R and a STAT3-inducible SEAP reporter gene (cat. Code: hkb-hil6, Invivogen) are used. The cell culture supernatant of the IL-6 stimulated samples with or without treatment is measured for bioactive human IL-6 to determine that due to the siRNA mediated interference, the cell culture supernatant with the treatment of Compounds B1, B2, B15, B16 and B17 leads to reduced bioactive human IL-6 compared to untreated control. The cell supernatant is used to quantitatively measure IFN-beta by ELISA (Human IFN-beta bioluminescent ELISA kit 2.0, Cat. Code: luex-hifnbv2, Invivogen).

### Example 9: Endogenous IL-6R Suppression Model in THP-1 Cells

In Vitro Transfection of THP-1 Cells with IL-6R Suppressing Compounds

A549 cells do not express IL-6R endogenously, therefore THP-1 cells are used due to their high endogenous expression of the receptor (54×, www.proteinatlas.org). Human monocyte leukemia cell line THP-1 (Sigma-Aldrich, Cat. #88081201) is maintained in growth medium (RPMI 1640 supplemented with 10% FBS and 2 mM glutamine). The cells are seeded at 30,000 THP-1 cells in a 96-well cell culture plate 72 hours before transfection, and activated with 50 nM of phorbol 12-myristate 13-acetate (PMA) (Sigma-Aldrich, Cat. #P8139) diluted in growth medium. The cells are transfected with Compounds B3-B5 (300-1200 ng/well) using Lipofectamine 2000 (Thermo Fisher Scientific). 100 μl of DMEM is removed from each well and replaced with 50 μl of Opti-MEM (Thermo Fisher Scientific) and 50 μl mRNA and Lipofectamine 2000 complex in Opti-MEM. After 5 hours, the medium is replaced with fresh growth medium supplemented with 50 nM PMA and the plates are incubated at 37° C. in a humidified atmosphere containing 5% CO₂ for 24 hours. After infection, cell culture supernatant (ThermoFisher Scientific, cat #BMS214) and cell lysate are processed (LSBio, cat #LS-F1001) to quantitatively detect IL-6R by ELISA. To verify the functional suppression of IL-6R, HEK-Blue™ IL-6 reporter cells stably transfected with IL-6R and a STAT3-inducible SEAP reporter gene (cat. Code: hkb-hil6, Invivogen) are used. Since transfection of Compounds B3-B5 leads to siRNA mediated suppression of IL-6R in HEK-Blue™ cells, the addition of recombinant human IL-6 (cat. Code:rcyec-hil6, Invivogen) does not activate the STAT-3 inducible SEAP reporter gene. This is an effective functional assay to validate the blockade of IL-6R signalling pathway. The cell supernatant is used to quantitatively measure IFN-beta by ELISA (Human IFN-beta bioluminescent ELISA kit 2.0, Cat. Code: luex-hifnbv2, Invivogen).

### Example 10: ACE2 Overexpression Model in A549 Cells

In Vitro Transfection of A549 Cells with ACE2 mRNA and ACE2 Suppressing/IFN-Beta Overexpression Compounds

An ACE2 overexpression model is used to evaluate simultaneous ACE2 RNA interference (RNAi) and IFN-beta overexpression by mRNA Compounds B6, B7, B15, B16 and B17 in A549 cells. The model is established by transfection with ACE2 mRNA (from SEQ ID NO: 57). Each sample of cells is co-transfected with one of the mRNA Compounds B6, B7, B15, B16 and B17 (300-900 ng/well), and ACE2 mRNA (300 ng/well). Post transfection, the cells are incubated at 37° C. in a humidified atmosphere containing 5% CO₂ for 24 hours, followed by quantification of ACE2 (target mRNA to downregulate) and IFN-beta (gene of interest to overexpress) by ELISA in the cell culture supernatant (Aviva Systems Biology, cat #OKBB00649).

### Example 11: SARS CoV-2 Spike Protein Overexpression Model in A549 Cells

In Vitro Transfection of A549 Cells with SARS CoV-2 Spike Protein mRNA and SARS CoV-2 Spike Protein Suppressing/IFN-Beta Overexpression Compounds

A SARS CoV-2 Spike (S) protein overexpression model is used to evaluate simultaneous SARS CoV-2 Spike protein RNA interference (RNAi) and IFN-beta overexpression by mRNA Compounds B8, B9, B11, B15, B16 and B17 in A549 cells. The model is established by transfection with mRNA encoding the receptor binding domain (RBD) of SARS CoV-2 spike protein (S-RBD, SEQ ID NO: 60). Each sample of cells is co-transfected with one of the mRNA Compounds B8, B9, B11, B15, B16 and B17 (300-900 ng/well), and S-RBD mRNA (300 ng/well). Post transfection, the cells are incubated at 37° C. in a humidified atmosphere containing 5% CO₂ for 24 hours, followed by quantification of S-RBD by ELISA (Sino biological, cat #KIT40591). Simultaneously, the IFN-beta expression is measured by ELISA in the cell culture supernatant (Human IFN-beta bioluminescent ELISA kit 2.0, Cat. Code: luex-hifnbv2, Invivogen).

### Example 12: SARS CoV-2 Nucleocapsid Protein Overexpression Model in A549 Cells

In Vitro Transfection of A549 Cells with SARS CoV-2 Nucleocapsid Protein mRNA and SARS CoV-2 Nucleocapsid Protein Suppressing/IFN-Beta Overexpression Compounds

A SARS CoV-2 Spike protein overexpression model is used to evaluate simultaneous SARS CoV-2 Nucleocapsid (N) protein RNAi suppression and IFN-beta overexpression by mRNA Compounds B8 and B10 in A549 cells. The model is established by transfection with mRNA encoding the complete coding domain of SARS CoV-2 N protein (SEQ ID NO: 62) tagged with 3′ eGFP. In a separate, additional, approach, the SARS CoV-2 N protein is overexpressed from a plasmid (pcDNA3+vector) thereby providing two independent systems to evaluate the effect of RNAi suppression by Compounds B8 and B10. The RNAi of Compounds B8 and B10 targeting SARS CoV-2 N protein disrupt the eGFP translation and expression.

Each sample of cells (mRNA-transfected cells or cells carrying the plasmid) is co-transfected with one of the mRNA Compounds B8 and B10 (300-900 ng/well), and SARS CoV-2 N mRNA (300 ng/well).

Post transfection, the cells are incubated at 37° C. in a humidified atmosphere containing 5% CO₂ for 24 hours, followed by quantification of SARS CoV-2 N protein by ELISA (Sino biological, cat #KIT40588). Simultaneously, the IFN-beta expression is measured by ELISA in the cell culture supernatant (Human IFN-beta bioluminescent ELISA kit 2.0, Cat. Code: luex-hifnbv2, Invivogen). To determine whether RNAi suppression by Compounds B8 and B10 leads to the disruption of eGFP translation, the SARS CoV-2 Nucleocapsid proteins tagged with eGFP (from expression of both plasmid and mRNA), are microscopically examined for eGFP expression using SpectraMax i3X multi-mode microplate reader (Molecular Devices). The percentage of eGFP positive cells is calculated in treated and control untreated samples.

### Example 13: SARS CoV-2 Nsp1 Overexpression Model in A549 Cells

In Vitro Transfection of A549 Cells with SARS CoV-2 Nonstructural Protein mRNA and SARS CoV-2 Nonstructural Protein Suppressing/IFN-Beta Overexpression Compounds

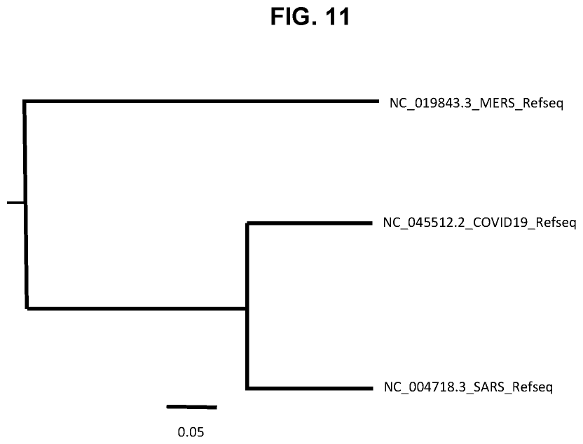

A genome sequence alignment of SARS CoV-2 with SARS CoV and MERS-CoV at the RNA level showed less conservation than an amino acid comparison. Phylogenetic tree analysis (Genetic distance model: Tamura-Nei; Tree build method: UPGMA) showed that MERS-CoV has high level of dissimilar RNA sequence (>45%) whereas SARS CoV and SARS CoV-2 exhibited low level of dissimilarity (up to 21%) (See FIG. 11). We aligned SARS CoV with SARS CoV-2 separately and searched for conserved minimum 20 bp loci for siRNA design. We identified a 47 bp homology near the beginning of viral genome (235-281 bp) which we used to design siRNA (Compounds B8 and B14). The siRNA is located at the first codon (ATG) of the non-structural protein 1 (Nsp1). Targeting the first codon (methionine; AUG) of viral genome ideally lead to huge impact on viral replication as next methionine (AUG) base located 84 amino acids distant to initiate alternative translation.

A SARS CoV-2 Nsp1 overexpression model is used to evaluate simultaneous SARS CoV-2 Nsp1 RNAi suppression and IFN-beta overexpression by mRNA Compounds B8 and B14 in A549 cells.

The model is established by transfection with mRNA encoding the partial domain (first 100 amino acids) of SARS CoV-2 Nsp1 (SEQ ID NO: 64) tagged with 3′ eGFP. In a separate, additional, approach, SARS CoV-2 Nsp1 is overexpressed from a plasmid (pcDNA3+vector) thereby providing two independent systems to evaluate the effect of RNAi suppression by Compounds B8 and B14. The RNAi of Compounds B8 and B14 targeting SARS CoV-2 Nsp1 disrupt the eGFP translation and expression.

Each sample of cells (mRNA-transfected cells or cells carrying the plasmid) is co-transfected with one of the mRNA Compounds B8 and B14 (300-900 ng/well), and SARS CoV-2 Nsp1 mRNA (300 ng/well).

Post transfection, the cells are incubated at 37° C. in a humidified atmosphere containing 5% CO₂ for 24 hours. To determine whether RNAi suppression by Compounds B8 and B14 leads to the disruption of eGFP translation, the SARS CoV-2 Nsp1 tagged with eGFP (from expression of both plasmid and mRNA), are microscopically examined for eGFP expression using SpectraMax i3X multi-mode microplate reader (Molecular Devices). The percentage of eGFP positive cells is calculated in treated and control untreated samples. Simultaneously, the IFN-beta expression is measured by ELISA in the cell culture supernatant (Human IFN-beta bioluminescent ELISA kit 2.0, Cat. Code: luex-hifnbv2, Invivogen). To determine whether RNAi suppression by Compounds B8 and B14 leads to the disruption of eGFP translation, the SARS CoV-2 Nucleocapsid proteins tagged with eGFP (from expression of both plasmid and mRNA), are microscopically examined for eGFP expression. The percentage of eGFP positive cells is calculated in treated and control untreated samples.

### Example 14: Design of Nsp12-Nsp13 siRNA Targeting SARS CoV-2, SARS-CoV and MERS-CoV mRNA, and Nsp12-Nsp13 Overexpression Model in A549 Cells

Design of Nsp12-Nsp13 siRNA Targeting SARS CoV-2, SARS-CoV and MERS-CoV mRNA

To design siRNAs that target all three of SARS CoV-2, SARS-CoV and MERS-CoV, we identified siRNA of as short as 17 bp, tolerating up to 1 mismatch among the sequences. Using this relaxed approach we designed one siRNA of 17 bp in length (between 14299-14318, referenced to SARS CoV-2 genome) and two additional siRNAs each having one bp mismatch tolerance among the three genomic sequences (15091-15107 and 17830-17849, referenced to SARS CoV-2 genome), combining them in a construct with IFN-beta overexpression.

A SARS CoV-2 Nsp12-13 overexpression model is used to evaluate simultaneous SARS CoV-2 Nsp12-13 RNAi suppression and IFN-beta overexpression by mRNA Compounds B12 and B13 in A549 cells. The model is established by transfection with mRNA encoding a non-coding domain of NSP12 and NSP13 (14202-17951 bp; 3749 bp) of SARS CoV-2 genome (SEQ ID NO: 67) tagged with 3′ eGFP. Each sample of cells (mRNA-transfected cells or cells carrying the plasmid) is co-transfected with one of the mRNA Compounds B12 and B13 (300-900 ng/well), and SARS CoV-2 NSP12 and NSP-13 partial genomic RNA (300 ng/well).

Post transfection, the cells are incubated at 37° C. in a humidified atmosphere containing 5% CO₂ for 24 hours, followed by Taqman-qPCR based assays to assess the viral RNA degradation, as compared to untransfected control. Simultaneously, the IFN-beta expression is measured by ELISA in the cell culture supernatant (Human IFN-beta bioluminescent ELISA kit 2.0, Cat. Code: luex-hifnbv2, Invivogen).

### Example 15: A549 Cell ACE2 Soluble Receptor Overexpression Model

In Vitro Transfection of A549 Cells with ACE2 Soluble Receptor Overexpression Compounds

A549 cells are typical alveolar type II (ATII) cells derived from human lung carcinoma. Since COVID-19 mortality primarily is associated with respiratory illness due to the high viral entry receptor (ACE2) expression in host ATII cells, A549 cells are used to mimic the clinical situation. The A549 cells (Sigma-Aldrich, Buchs Switzerland Cat. #6012804) are maintained on Dulbecco's Modified Eagle's medium-high glucose (DMEM, Sigma-Aldrich, Buchs Switzerland cat #D0822) supplemented with 10% FBS (Thermofischer, Basel, Switzerland cat #10500-064). To assess the ACE2 soluble receptor expression the A549 cells are plated at a density of 10,000 cells/well in a regular growth medium 24 hours prior to transfection. Thereafter, cells are transfected with Compounds B18 and B19 (0.3-0.6 micrograms) using Lipofectamine 2000 (www.invitrogen.com) following the manufacturer's instructions. 100 μl of DMEM are removed and 50 μl of Opti-MEM (www.thermofisher.com) are added to each well followed by 50 μl mRNA and Lipofectamine 2000 complex in Opti-MEM. After 5 hours of incubation, the medium is replaced by fresh growth medium and the plates are incubated for 24 hours at 37° C. in a humidified atmosphere containing 5% CO₂ followed by ACE2 quantification by ELISA (Aviva Systems Biology, cat #OKBB00649). The anti-viral activity of Compound B18 and Compound B19 are investigated in Examples 11-13.

### Example 16: Additional Constructs

Construct Design, Sequence, and Synthesis

Details of construct design and synthesis are described in Example 1. Table 8 summarizes additional compounds used in the examples in the present disclosure with their respective siRNA target to downregulate protein expression, and protein target for upregulated protein expression. The sequences of the constructs of A9-A15 are shown in Table 9 and annotated as indicated in the table below. All uridines in Compounds A9-A15 used in the examples described herein were modified to N¹-methylpseudouridine. For each compound, the position of siRNA sequence is indicated in regard to the gene of interest. For example, “5′ siRNA position” indicates that siRNA sequences are upstream of or 5′ to the gene of interest in the compound. Conversely, “3′ siRNA position” indicates that siRNA sequences are downstream of or 3′ to the gene of interest in the compound. The plasmid sequences of the constructs of A9-A15 are shown in Table 10.

TABLE 8
Summary of Compounds A9-A15

| Compound | siRNA | siRNA | # of | Protein Target | |
|---|---|---|---|---|---|
| ID | Target | Position | siRNAs | (gene of interest) | Indication |
| A9 | TNF-alpha | 5’ | 3 | IL-4 | Psoriasis |
| A10 | TNF-alpha | 3’ | 3 | IL-4 | Psoriasis |
| A11 | ALK2 | 3’ | 3 | IGF-1 | FOP |
| A12 | SOD1 | 5’ | 3 | IGF-1 | ALS |
| A13 | SOD1 | 5’ | 3 | EPO | ALS |
| A14 | IL-1 beta | 5’ | 3 | IGF-1 | OA, IVDD |
| A15 | IL-1 beta | 3’ | 3 | IGF-1 | OA, IVDD |

FOP: Fibrodysplasia ossificans progressiva;
ALS: Amyotrophic lateral sclerosis;
OA: Osteoarthritis;
IVDD: Intervertebral disc disease

TABLE 9
Sequences of Compounds A9-A15

| SEQ ID NO: | Compound # | Sequence (5′→3′ direction) |
|---|---|---|
| 152 | Compound A9 | ATAGTGAGTCGTATTAACGTACCAACAAGGCGTGGAGCTGAGAGATAAA |
| 5′ to 3′: | | CTTG [symbol] TTTATCTTAGAGGCATATCCCTACG |
| A9-1 sense | | TACCAACAAGGGCCTGTACCTCATCTACTACTTG [symbol] |
| strand siRNA | | [symbol] TTTATCTTAGAGGCATATCCCTACGTACCAACAAGGTATGAGCC |
| 87, antisense | | CATCTATCTACTTG [symbol] TTTATCTTAGAGGCAT |
| 117; | | ATCCCTGCCACCATGGGACTGACATCTCAACTGCTGCCTCCACTGTTCT |
| A9-2 sense | | TTCTGCTGGCCTGCGCCGGCAATTTTGTGCACGGCCACAAGTGCGACAT |
| strand siRNA | | CACCCTGCAAGAGATCATCAAGACCCTGAACAGCCTGACCGAGCAGAAA |
| 88, antisense | | ACCCTGTGCACCGAGCTGACCGTGACCGATATCTTTGCCGCCAGCAAGA |
| 118; | | ACACAACCGAGAAAGAGACATTCTGCAGAGCCGCCACCGTGCTGAGACA |
| A9-3 sense | | GTTCTACAGCCACCACGAGAAGGACACCAGATGCCTGGGAGCTACAGCC |
| strand siRNA | | CAGCAGTTCCACAGACACAAGCAGCTGATCCGGTTCCTGAAGCGGCTGG |
| 89, antisense | | ACAGAAATCTGTGGGGACTCGCCGGCCTGAATAGCTGCCCTGTGAAAGA |
| 119 | | GGCCAACCAGTCTACCCTGGAAAACTTCCTGGAACGGCTGAAAACCATC |
| | | ATGCGCGAGAAGTACAGCAAGTGCAGCAGCTGATTTATCTTAGAGGCAT |
| | | ATCCCT |
| 153 | Compound A10 | GCCACCATGGGACTGACATCTCAACTGCTGCCTCCACTGTTCTTTCTGC |
| 5′ to 3′: | | TGGCCTGCGCCGGCAATTTTGTGCACGGCCACAAGTGCGACATCACCCT |
| A10-1 sense | | GCAAGAGATCATCAAGACCCTGAACAGCCTGACCGAGCAGAAAACCCTG |
| strand siRNA | | TGCACCGAGCTGACCGTGACCGATATCTTTGCCGCCAGCAAGAACACAA |
| 87, antisense | | CCGAGAAAGAGACATTCTGCAGAGCCGCCACCGTGCTGAGACAGTTCTA |
| 117; | | CAGCCACCACGAGAAGGACACCAGATGCCTGGGAGCTACAGCCCAGCAG |
| A10-2 sense | | TTCCACAGACACAAGCAGCTGATCCGGTTCCTGAAGCGGCTGGACAGAA |
| strand siRNA | | ATCTGTGGGGACTCGCCGGCCTGAATAGCTGCCCTGTGAAAGAGGCCAA |
| 88, antisense | | CCAGTCTACCCTGGAAAACTTCCTGGAACGGCTGAAAACCATCATGCGC |
| 118; | | GAGAAGTACAGCAAGTGCAGCAGCTGAATAGTGAGTCGTATTAACGTAC |
| A10-3 sense | | CAACAAGGCGTGGAGCTGAGAGATAAACTTG [symbol] |
| strand siRNA | | [symbol] TTTATCTTAGAGGCATATCCCTACGTACCAACAAGGGCCTGTACCTC |
| 89, antisense | | ATCTACTACTTG [symbol] TTTATCTTAGAGGCATA |
| 119 | | TCCCTACGTACCAACAAGGTATGAGCCCATCTATCTACTTG [symbol] |
| | | [symbol] TTTATCTTAGAGGCATATCCCTTTTATCTTAGAGGCAT |
| | | ATCCCT |
| 154 | Compound A11 | GCCACCATGACCATCCTGTTTCTGACAATGGTCATCAGCTACTTCGGCT |
| 5′ to 3′: | | GCATGAAGGCCGTGAAGATGCACACCATGAGCAGCAGCCACCTGTTCTA |
| A11-1 sense | | TCTGGCCCTGTGCCTGCTGACCTTTACCAGCTCTGCTACCGCCGGACCT |
| strand siRNA | | GAGACACTTTGTGGCGCTGAACTGGTGGACGCCCTGCAGTTTGTGTGTG |
| 140, | | GCGACAGAGGCTTCTACTTCAACAAGCCCACAGGCTACGGCAGCAGCTC |
| antisense | | TAGAAGGGCTCCTCAGACCGGAATCGTGGACGAGTGCTGCTTCAGAAGC |
| 146; A11-2 | | TGCGACCTGCGGCGGCTGGAAATGTATTGTGCCCCTCTGAAGCCTGCCA |
| sense strand | | AGAGCGCCTAAATAGTGAGTCGTATTAACGTACCAACAAGGCCTCATTA |
| siRNA 141, | | TTCTCTCTACTTG [symbol] TTTATCTTAGAGGCATAT |
| antisense | | CCCTACGTACCAACAAGTGTTCGCAGTATGTCTTACTTG [symbol] |
| 147; A11-3 | | [symbol] TTTATCTTAGAGGCATATCCCTACGTACCAACAAGCCTGCC |
| sense strand | | TGCTGGGAGTTACTTG [symbol] TTTATCTTAGAGGCA |
| siRNA 142, | | TATCCCTTTTATCTTAGAGGCATATCCCT |
| antisense 148 | | |
| 155 | Compound A12 | ATAGTGAGTCGTATTAACGTACCAACAAGAAGGAAAGTAATGGACCAGT |
| 5′ to 3′: | | ACTTG [symbol] TTTATCTTAGAGGCATATCCCTA |
| A12-1 sense | | CGTACCAACAAGGTCCTCACTTTAATCCTCTAACTTG [symbol] |
| strand siRNA | | [symbol] TTTATCTTAGAGGCATATCCCTACGTACCAACAAGGAGAC |
| 143, | | TTGGGCAATGTGACTACTTG [symbol] TTTATCTT |
| antisense | | AGAGGCATATCCCTGCCACCATGGGCAAGATTAGCAGCCTGCCTACACA |
| 149; A12-2 | | GCTGTTCAAGTGCTGCTTCTGCGACTTCCTGAAAGTGAAGATGCACACC |
| sense strand | | ATGAGCAGCAGCCACCTGTTCTATCTGGCCCTGTGCCTGCTGACCTTTA |
| siRNA 144, | | CCAGCTCTGCTACCGCCGGACCTGAGACACTTTGTGGCGCTGAACTGGT |
| antisense | | GGACGCCCTGCAGTTTGTGTGTGGCGACAGAGGCTTCTACTTCAACAAG |
| 150; A12-3 | | CCCACAGGCTACGGCAGCAGCTCTAGAAGGGCTCCTCAGACCGGAATCG |
| sense strand | | TGGACGAGTGCTGTTTCAGAAGCTGCGACCTGCGGCGGCTGGAAATGTA |
| siRNA 145, | | TTGTGCCCCTCTGAAGCCTGCCAAGAGCGCCTAATTTATCTTAGAGGCA |
| antisense 151 | | TATCCCT |
| 156 | Compound A13 | ATAGTGAGTCGTATTAACGTACCAACAAGAAGGAAAGTAATGGACCAGT |
| 5′ to 3′: | | ACTTG [symbol] TTTATCTTAGAGGCATATCCCTA |
| A13-1 sense | | CGTACCAACAAGGTCCTCACTTTAATCCTCTAACTTG [symbol] |
| strand siRNA | | [symbol] TTTATCTTAGAGGCATATCCCTACGTACCAACAAGGAGAC |
| 143, | | TTGGGCAATGTGACTACTTG [symbol] TTTATCTT |
| antisense | | AGAGGCATATCCCTGCCACCATGGGAGTGCATGAATGTCCTGCTTGGCT |
| 149; A13-2 | | GTGGCTGCTGCTGAGCCTGCTGTCTCTGCCTCTGGGACTGCCTGTTCTT |
| sense strand | | GGAGCCCCTCCTAGACTGATCTGCGACAGCAGAGTGCTGGAAAGATACC |
| siRNA 144, | | TGCTGGAAGCCAAAGAGGCCGAGAACATCACCACAGGCTGTGCCGAGCA |
| antisense | | CTGCAGCCTGAACGAGAATATCACCGTGCCTGAGACCAAAGTGAACTTC |
| 150; A13-3 | | TACGCCTGGAAGCGGATGGAAGTGGGCCAGCAGGCTGTGGAAGTTTGGC |
| sense strand | | AAGGACTGGCCCTGCTGAGCGAAGCTGTTCTGAGAGGACAGGCTCTGCT |
| siRNA 145, | | GGTCAACAGCTCTCAGCCTTGGGAACCTCTGCAACTGCACGTGGACAAG |
| antisense 151 | | GCCGTGTCTGGCCTGAGAAGCCTGACCACACTGCTGAGAGCACTGGGAG |
| | | CCCAGAAAGAGGCCATCTCTCCACCTGATGCTGCCTCTGCTGCCCCTCT |
| | | GAGAACCATCACCGCCGACACCTTCAGAAAGCTGTTCCGGGTGTACAGC |
| | | AACTTCCTGCGGGGCAAGCTGAAGCTGTACACAGGCGAGGCTTGCAGAA |
| | | CCGGCGACAGATAATTTATCTTAGAGGCATATCCCT |
| 157 | Compound A14 | ATAGTGAGTCGTATTAACGTACCAACAAGAAAGATGATAAGCCCACTCT |
| 5′ to 3′: | | ACTTG [symbol] TTTATCTTAGAGGCATATCCCTA |
| A14-1 sense | | CGTACCAACAAGGTGATGTCTGGTCCATATGAACTTG [symbol] |
| strand siRNA | | [symbol] TTTATCTTAGAGGCATATCCCTACGTACCAACAAGATGAT |
| 84, antisense | | AAGCCCACTCTAACTTG [symbol] TTTATCTTAGAGGC |
| 114; A14-2 | | ATATCCCTGCCACCATGGGCAAGATTAGCAGCCTGCCTACACAGCTGTT |
| sense strand | | CAAGTGCTGCTTCTGCGACTTCCTGAAAGTGAAGATGCACACCATGAGC |
| siRNA 85, | | AGCAGCCACCTGTTCTATCTGGCCCTGTGCCTGCTGACCTTTACCAGCT |
| antisense | | CTGCTACCGCCGGACCTGAGACACTTTGTGGCGCTGAACTGGTGGACGC |
| 115; A14-3 | | CCTGCAGTTTGTGTGTGGCGACAGAGGCTTCTACTTCAACAAGCCCACA |
| sense strand | | GGCTACGGCAGCAGCTCTAGAAGGGCTCCTCAGACCGGAATCGTGGACG |
| siRNA 86, | | AGTGCTGTTTCAGAAGCTGCGACCTGCGGCGGCTGGAAATGTATTGTGC |
| antisense 116 | | CCCTCTGAAGCCTGCCAAGAGCGCCTAATTTATCTTAGAGGCATATCCC |
| | | T |
| 158 | Compound A15 | GCCACCATGGGCAAGATTAGCAGCCTGCCTACACAGCTGTTCAAGTGCT |
| 5′ to 3′: | | GCTTCTGCGACTTCCTGAAAGTGAAGATGCACACCATGAGCAGCAGCCA |
| A15-1 sense | | CCTGTTCTATCTGGCCCTGTGCCTGCTGACCTTTACCAGCTCTGCTACC |
| strand siRNA | | GCCGGACCTGAGACACTTTGTGGCGCTGAACTGGTGGACGCCCTGCAGT |
| 84, antisense | | TTGTGTGTGGCGACAGAGGCTTCTACTTCAACAAGCCCACAGGCTACGG |
| 114; A15-2 | | CAGCAGCTCTAGAAGGGCTCCTCAGACCGGAATCGTGGACGAGTGCTGT |
| sense strand | | TTCAGAAGCTGCGACCTGCGGCGGCTGGAAATGTATTGTGCCCCTCTGA |
| siRNA 85, | | AGCCTGCCAAGAGCGCCTAAATAGTGAGTCGTATTAACGTACCAACAAG |
| antisense | | AAAGATGATAAGCCCACTCTACTTG [symbol] TTT |
| 115; A15-3 | | ATCTTAGAGGCATATCCCTACGTACCAACAAGGTGATGTCTGGTCCATA |
| sense strand | | TGAACTTG [symbol] TTTATCTTAGAGGCATATCC |
| siRNA 86, | | CTACGTACCAACAAGATGATAAGCCCACTCTAACTTG [symbol] |
| antisense 116 | | [symbol] TTTATCTTAGAGGCATATCCCTTTTATCTTAGAGGCATATCCC |
| | | T |

Bold = Sense siRNA strand
Bold and Italics = anti-Sense siRNA strand
Underline = Signal peptide
Italics = Kozak sequence

TABLE 10
Plasmid Sequences for Compounds A9-A15

| SEQ ID NO | Compound # | Sequence (5′→3′ direction) |
|---|---|---|
| 160 | Compound A9 in | CTAAATTGTAAGCGTTAATATTTTGTTAAAATTCGCGTTAAATTTTTGT |
| | pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGGCGTGGAGCTGAGAGATAAACTTGTTATCTCTCAGCTCCACGC |
| | | CTTTATCTTAGAGGCATATCCCTACGTACCAACAAGGGCCTGTACCTCA |
| | | TCTACTACTTGAGTAGATGAGGTACAGGCCCTTTATCTTAGAGGCATAT |
| | | CCCTACGTACCAACAAGGTATGAGCCCATCTATCTACTTGAGATAGATG |
| | | GGCTCATACCTTTATCTTAGAGGCATATCCCTGCCACCATGGGACTGAC |
| | | ATCTCAACTGCTGCCTCCACTGTTCTTTCTGCTGGCCTGCGCCGGCAAT |
| | | TTTGTGCACGGCCACAAGTGCGACATCACCCTGCAAGAGATCATCAAGA |
| | | CCCTGAACAGCCTGACCGAGCAGAAAACCCTGTGCACCGAGCTGACCGT |
| | | GACCGATATCTTTGCCGCCAGCAAGAACACAACCGAGAAAGAGACATTC |
| | | TGCAGAGCCGCCACCGTGCTGAGACAGTTCTACAGCCACCACGAGAAGG |
| | | ACACCAGATGCCTGGGAGCTACAGCCCAGCAGTTCCACAGACACAAGCA |
| | | GCTGATCCGGTTCCTGAAGCGGCTGGACAGAAATCTGTGGGGACTCGCC |
| | | GGCCTGAATAGCTGCCCTGTGAAAGAGGCCAACCAGTCTACCCTGGAAA |
| | | ACTTCCTGGAACGGCTGAAAACCATCATGCGCGAGAAGTACAGCAAGTG |
| | | CAGCAGCTGATTTATCTTAGAGGCATATCCCTCTGGGCCTCATGGGCCT |
| | | TCCGCTCACTGCCCGCTTTCCAGTCGGGAAACCTGTCGTGCCAGCTGCA |
| | | TTAACATGGTCATAGCTGTTTCCTTGCGTATTGGGCGCTCTCCGCTTCC |
| | | TCGCTCACTGACTCGCTGCGCTCGGTCGTTCGGGTAAAGCCTGGGGTGC |
| | | CTAATGAGCAAAAGGCCAGCAAAAGGCCAGGAACCGTAAAAAGGCCGCG |
| | | TTGCTGGCGTTTTTCCATAGGCTCCGCCCCCCTGACGAGCATCACAAAA |
| | | ATCGACGCTCAAGTCAGAGGTGGCGAAACCCGACAGGACTATAAAGATA |
| | | CCAGGCGTTTCCCCCTGGAAGCTCCCTCGTGCGCTCTCCTGTTCCGACC |
| | | CTGCCGCTTACCGGATACCTGTCCGCCTTTCTCCCTTCGGGAAGCGTGG |
| | | CGCTTTCTCATAGCTCACGCTGTAGGTATCTCAGTTCGGTGTAGGTCGT |
| | | TCGCTCCAAGCTGGGCTGTGTGCACGAACCCCCCGTTCAGCCCGACCGC |
| | | TGCGCCTTATCCGGTAACTATCGTCTTGAGTCCAACCCGGTAAGACACG |
| | | ACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGC |
| | | TACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGC |
| | | TGGTAGCGGTGGTTTTTTTGTTTGCAAGCAGCAGATTACGCGCAGAAAA |
| | | AAAGGATCTCAAGAAGATCCTTTGATCTTTTCTACGGGGTCTGACGCTC |
| | | AGTGGAACGAAAACTGAGGTTAAGGGATTTTGGTCATGAGATTATCAAA |
| | | AAGGATCTTCACCTAGATCCTTTTAAATTAAAAATGAAGTTTTAAATCA |
| | | ATCTAAAGTATATATGAGTAAACTTGGTCTGACAGTTACCAATGCTTAA |
| | | TCAGTGAGGCACCTATCTCAGCGATCTGTCTATTTCGTTCATCCATAGT |
| | | TGCCTGACTCCCCGTCGTGTAGATAACTACGATACGGGAGGGCTTACCA |
| | | TCTGGCCCCAGTGCTGCAATGATACCGCGAGAACCACGCTCACCGGCTC |
| | | CAGATTTATCAGCAATAAACCAGCCAGCCGGAAGGGCCGAGCGCAGAAG |
| | | TGGTCCTGCAACTTTATCCGCCTCCATCCAGTCTATTAATTGTTGCCGG |
| | | GAAGCTAGAGTAAGTAGTTCGCCAGTTAATAGTTTGCGCAACGTTGTTG |
| | | CCATTGCTACAGGCATCGTGGTGTCACGCTCGTCGTTTGGTATGGCTTC |
| | | ATTCAGCTCCGGTTCCCAACGATCAAGGCGAGTTACATGATCCCCCATG |
| | | TTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAA |
| | | TTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCT |
| | | CTTGCCCGGCGTCAATACGGGATAATACCGCGCCACATAGCAGAACTTT |
| | | AAAAGTGCTCATCATTGGAAAACGTTCTTCGGGGCGAAAACTCTCAAGG |
| | | ATCTTACCGCTGTTGAGATCCAGTTCGATGTAACCCACTCGTGCACCCA |
| | | ACTGATCTTCAGCATCTTTTACTTTCACCAGCGTTTCTGGGTGAGCAAA |
| | | AACAGGAAGGCAAAATGCCGCAAAAAAGGGAATAAGGGCGACACGGAAA |
| | | TGTTGAATACTCATACTCTTCCTTTTTCAATATTATTGAAGCATTTATC |
| | | AGGGTTATTGTCTCATGAGCGGATACATATTTGAATGTATTTAGAAAAA |
| | | TAAACAAATAGGGGTTCCGCGCACATTTCCCCGAAAAGTGCCAC |
| 161 | Compound A10 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATGCCACCATGGGACTGACATCTCA |
| | | ACTGCTGCCTCCACTGTTCTTTCTGCTGGCCTGCGCCGGCAATTTTGTG |
| | | CACGGCCACAAGTGCGACATCACCCTGCAAGAGATCATCAAGACCCTGA |
| | | ACAGCCTGACCGAGCAGAAAACCCTGTGCACCGAGCTGACCGTGACCGA |
| | | TATCTTTGCCGCCAGCAAGAACACAACCGAGAAAGAGACATTCTGCAGA |
| | | GCCGCCACCGTGCTGAGACAGTTCTACAGCCACCACGAGAAGGACACCA |
| | | GATGCCTGGGAGCTACAGCCCAGCAGTTCCACAGACACAAGCAGCTGAT |
| | | CCGGTTCCTGAAGCGGCTGGACAGAAATCTGTGGGGACTCGCCGGCCTG |
| | | AATAGCTGCCCTGTGAAAGAGGCCAACCAGTCTACCCTGGAAAACTTCC |
| | | TGGAACGGCTGAAAACCATCATGCGCGAGAAGTACAGCAAGTGCAGCAG |
| | | CTGAATAGTGAGTCGTATTAACGTACCAACAAGGCGTGGAGCTGAGAGA |
| | | TAAACTTGTTATCTCTCAGCTCCACGCCTTTATCTTAGAGGCATATCCC |
| | | TACGTACCAACAAGGGCCTGTACCTCATCTACTACTTGAGTAGATGAGG |
| | | TACAGGCCCTTTATCTTAGAGGCATATCCCTACGTACCAACAAGGTATG |
| | | AGCCCATCTATCTACTTGAGATAGATGGGCTCATACCTTTATCTTAGAG |
| | | GCATATCCCTTTTATCTTAGAGGCATATCCCTCTGGGCCTCATGGGCCT |
| | | TCCGCTCACTGCCCGCTTTCCAGTCGGGAAACCTGTCGTGCCAGCTGCA |
| | | TTAACATGGTCATAGCTGTTTCCTTGCGTATTGGGCGCTCTCCGCTTCC |
| | | TCGCTCACTGACTCGCTGCGCTCGGTCGTTCGGGTAAAGCCTGGGGTGC |
| | | CTAATGAGCAAAAGGCCAGCAAAAGGCCAGGAACCGTAAAAAGGCCGCG |
| | | TTGCTGGCGTTTTTCCATAGGCTCCGCCCCCCTGACGAGCATCACAAAA |
| | | ATCGACGCTCAAGTCAGAGGTGGCGAAACCCGACAGGACTATAAAGATA |
| | | CCAGGCGTTTCCCCCTGGAAGCTCCCTCGTGCGCTCTCCTGTTCCGACC |
| | | CTGCCGCTTACCGGATACCTGTCCGCCTTTCTCCCTTCGGGAAGCGTGG |
| | | CGCTTTCTCATAGCTCACGCTGTAGGTATCTCAGTTCGGTGTAGGTCGT |
| | | TCGCTCCAAGCTGGGCTGTGTGCACGAACCCCCCGTTCAGCCCGACCGC |
| | | TGCGCCTTATCCGGTAACTATCGTCTTGAGTCCAACCCGGTAAGACACG |
| | | ACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGC |
| | | TACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGC |
| | | TGGTAGCGGTGGTTTTTTTGTTTGCAAGCAGCAGATTACGCGCAGAAAA |
| | | AAAGGATCTCAAGAAGATCCTTTGATCTTTTCTACGGGGTCTGACGCTC |
| | | AGTGGAACGAAAACTCACGTTAAGGGATTTTGGTCATGAGATTATCAAA |
| | | AAGGATCTTCACCTAGATCCTTTTAAATTAAAAATGAAGTTTTAAATCA |
| | | ATCTAAAGTATATATGAGTAAACTTGGTCTGACAGTTACCAATGCTTAA |
| | | TCAGTGAGGCACCTATCTCAGCGATCTGTCTATTTCGTTCATCCATAGT |
| | | TGCCTGACTCCCCGTCGTGTAGATAACTACGATACGGGAGGGCTTACCA |
| | | TCTGGCCCCAGTGCTGCAATGATACCGCGAGAACCACGCTCACCGGCTC |
| | | CAGATTTATCAGCAATAAACCAGCCAGCCGGAAGGGCCGAGCGCAGAAG |
| | | TGGTCCTGCAACTTTATCCGCCTCCATCCAGTCTATTAATTGTTGCCGG |
| | | GAAGCTAGAGTAAGTAGTTCGCCAGTTAATAGTTTGCGCAACGTTGTTG |
| | | CCATTGCTACAGGCATCGTGGTGTCACGCTCGTCGTTTGGTATGGCTTC |
| | | ATTCAGCTCCGGTTCCCAACGATCAAGGCGAGTTACATGATCCCCCATG |
| | | TTGTGCAAAAAAGCGGTTAGCTCCTTCGGTCCTCCGATCGTTGTCAGAA |
| | | GTAAGTTGGCCGCAGTGTTATCACTCATGGTTATGGCAGCACTGCATAA |
| | | TTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCT |
| | | CTTGCCCGGCGTCAATACGGGATAATACCGCGCCACATAGCAGAACTTT |
| | | AAAAGTGCTCATCATTGGAAAACGTTCTTCGGGGCGAAAACTCTCAAGG |
| | | ATCTTACCGCTGTTGAGATCCAGTTCGATGTAACCCACTCGTGCACCCA |
| | | ACTGATCTTCAGCATCTTTTACTTTCACCAGCGTTTCTGGGTGAGCAAA |
| | | AACAGGAAGGCAAAATGCCGCAAAAAAGGGAATAAGGGCGACACGGAAA |
| | | TGTTGAATACTCATACTCTTCCTTTTTCAATATTATTGAAGCATTTATC |
| | | AGGGTTATTGTCTCATGAGCGGATACATATTTGAATGTATTTAGAAAAA |
| | | TAAACAAATAGGGGTTCCGCGCACATTTCCCCGAAAAGTGCCAC |
| 162 | Compound A11 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATGCCACCATGACCATCCTGTTTCT |
| | | GACAATGGTCATCAGCTACTTCGGCTGCATGAAGGCCGTGAAGATGCAC |
| | | ACCATGAGCAGCAGCCACCTGTTCTATCTGGCCCTGTGCCTGCTGACCT |
| | | TTACCAGCTCTGCTACCGCCGGACCTGAGACACTTTGTGGCGCTGAACT |
| | | GGTGGACGCCCTGCAGTTTGTGTGTGGCGACAGAGGCTTCTACTTCAAC |
| | | AAGCCCACAGGCTACGGCAGCAGCTCTAGAAGGGCTCCTCAGACCGGAA |
| | | TCGTGGACGAGTGCTGCTTCAGAAGCTGCGACCTGCGGCGGCTGGAAAT |
| | | GTATTGTGCCCCTCTGAAGCCTGCCAAGAGCGCCTAAATAGTGAGTCGT |
| | | ATTAACGTACCAACAAGGCCTCATTATTCTCTCTACTTGAGAGAGAATA |
| | | ATGAGGCCTTTATCTTAGAGGCATATCCCTACGTACCAACAAGTGTTCG |
| | | CAGTATGTCTTACTTGAAGACATACTGCGAACACTTTATCTTAGAGGCA |
| | | TATCCCTACGTACCAACAAGCCTGCCTGCTGGGAGTTACTTGAACTCCC |
| | | AGCAGGCAGGCTTTATCTTAGAGGCATATCCCTTTTATCTTAGAGGCAT |
| | | ATCCCTCTGGGCCTCATGGGCCTTCCGCTCACTGCCCGCTTTCCAGTCG |
| | | GGAAACCTGTCGTGCCAGCTGCATTAACATGGTCATAGCTGTTTCCTTG |
| | | CGTATTGGGCGCTCTCCGCTTCCTCGCTCACTGACTCGCTGCGCTCGGT |
| | | CGTTCGGGTAAAGCCTGGGGTGCCTAATGAGCAAAAGGCCAGCAAAAGG |
| | | CCAGGAACCGTAAAAAGGCCGCGTTGCTGGCGTTTTTCCATAGGCTCCG |
| | | CCCCCCTGACGAGCATCACAAAAATCGACGCTCAAGTCAGAGGTGGCGA |
| | | AACCCGACAGGACTATAAAGATACCAGGCGTTTCCCCCTGGAAGCTCCC |
| | | TCGTGCGCTCTCCTGTTCCGACCCTGCCGCTTACCGGATACCTGTCCGC |
| | | CTTTCTCCCTTCGGGAAGCGTGGCGCTTTCTCATAGCTCACGCTGTAGG |
| | | TATCTCAGTTCGGTGTAGGTCGTTCGCTCCAAGCTGGGCTGTGTGCACG |
| | | AACCCCCCGTTCAGCCCGACCGCTGCGCCTTATCCGGTAACTATCGTCT |
| | | TGAGTCCAACCCGGTAAGACACGACTTATCGCCACTGGCAGCAGCCACT |
| | | GGTAACAGGATTAGCAGAGCGAGGTATGTAGGCGGTGCTACAGAGTTCT |
| | | TGAAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCTCT |
| | | TGATCCGGCAAACAAACCACCGCTGGTAGCGGTGGTTTTTTTGTTTGCA |
| | | AGCAGCAGATTACGCGCAGAAAAAAAGGATCTCAAGAAGATCCTTTGAT |
| | | CTTTTCTACGGGGTCTGACGCTCAGTGGAACGAAAACTCACGTTAAGGG |
| | | ATTTTGGTCATGAGATTATCAAAAAGGATCTTCACCTAGATCCTTTTAA |
| | | ATTAAAAATGAAGTTTTAAATCAATCTAAAGTATATATGAGTAAACTTG |
| | | GTCTGACAGTTACCAATGCTTAATCAGTGAGGCACCTATCTCAGCGATC |
| | | TGTCTATTTCGTTCATCCATAGTTGCCTGACTCCCCGTCGTGTAGATAA |
| | | CTACGATACGGGAGGGCTTACCATCTGGCCCCAGTGCTGCAATGATACC |
| | | GCGAGAACCACGCTCACCGGCTCCAGATTTATCAGCAATAAACCAGCCA |
| | | GCCGGAAGGGCCGAGCGCAGAAGTGGTCCTGCAACTTTATCCGCCTCCA |
| | | TCCAGTCTATTAATTGTTGCCGGGAAGCTAGAGTAAGTAGTTCGCCAGT |
| | | TAATAGTTTGCGCAACGTTGTTGCCATTGCTACAGGCATCGTGGTGTCA |
| | | CGCTCGTCGTTTGGTATGGCTTCATTCAGCTCCGGTTCCCAACGATCAA |
| | | GGCGAGTTACATGATCCCCCATGTTGTGCAAAAAAGCGGTTAGCTCCTT |
| | | CGGTCCTCCGATCGTTGTCAGAAGTAAGTTGGCCGCAGTGTTATCACTC |
| | | ATGGTTATGGCAGCACTGCATAATTCTCTTACTGTCATGCCATCCGTAA |
| | | GATGCTTTTCTGTGACTGGTGAGTACTCAACCAAGTCATTCTGAGAATA |
| | | GTGTATGCGGCGACCGAGTTGCTCTTGCCCGGCGTCAATACGGGATAAT |
| | | ACCGCGCCACATAGCAGAACTTTAAAAGTGCTCATCATTGGAAAACGTT |
| | | CTTCGGGGCGAAAACTCTCAAGGATCTTACCGCTGTTGAGATCCAGTTC |
| | | GATGTAACCCACTCGTGCACCCAACTGATCTTCAGCATCTTTTACTTTC |
| | | ACCAGCGTTTCTGGGTGAGCAAAAACAGGAAGGCAAAATGCCGCAAAAA |
| | | AGGGAATAAGGGCGACACGGAAATGTTGAATACTCATACTCTTCCTTTT |
| | | TCAATATTATTGAAGCATTTATCAGGGTTATTGTCTCATGAGCGGATAC |
| | | ATATTTGAATGTATTTAGAAAAATAAACAAATAGGGGTTCCGCGCACAT |
| | | TTCCCCGAAAAGTGCCAC |
| 163 | Compound A12 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGAAGGAAAGTAATGGACCAGTACTTGACTGGTCCATTACTTTCC |
| | | TTCTTTATCTTAGAGGCATATCCCTACGTACCAACAAGGTCCTCACTTT |
| | | AATCCTCTAACTTGTAGAGGATTAAAGTGAGGACCTTTATCTTAGAGGC |
| | | ATATCCCTACGTACCAACAAGGAGACTTGGGCAATGTGACTACTTGAGT |
| | | CACATTGCCCAAGTCTCCTTTATCTTAGAGGCATATCCCTGCCACCATG |
| | | GGCAAGATTAGCAGCCTGCCTACACAGCTGTTCAAGTGCTGCTTCTGCG |
| | | ACTTCCTGAAAGTGAAGATGCACACCATGAGCAGCAGCCACCTGTTCTA |
| | | TCTGGCCCTGTGCCTGCTGACCTTTACCAGCTCTGCTACCGCCGGACCT |
| | | GAGACACTTTGTGGCGCTGAACTGGTGGACGCCCTGCAGTTTGTGTGTG |
| | | GCGACAGAGGCTTCTACTTCAACAAGCCCACAGGCTACGGCAGCAGCTC |
| | | TAGAAGGGCTCCTCAGACCGGAATCGTGGACGAGTGCTGTTTCAGAAGC |
| | | TGCGACCTGCGGCGGCTGGAAATGTATTGTGCCCCTCTGAAGCCTGCCA |
| | | AGAGCGCCTAATTTATCTTAGAGGCATATCCCTCTGGGCCTCATGGGCC |
| | | TTCCGCTCACTGCCCGCTTTCCAGTCGGGAAACCTGTCGTGCCAGCTGC |
| | | ATTAACATGGTCATAGCTGTTTCCTTGCGTATTGGGCGCTCTCCGCTTC |
| | | CTCGCTCACTGACTCGCTGCGCTCGGTCGTTCGGGTAAAGCCTGGGGTG |
| | | CCTAATGAGCAAAAGGCCAGCAAAAGGCCAGGAACCGTAAAAAGGCCGC |
| | | GTTGCTGGCGTTTTTCCATAGGCTCCGCCCCCCTGACGAGCATCACAAA |
| | | AATCGAGGCTCAAGTCAGAGGTGGCGAAACCCGACAGGACTATAAAGAT |
| | | ACCAGGCGTTTCCCCCTGGAAGCTCCCTCGTGCGCTCTCCTGTTCCGAC |
| | | CCTGCCGCTTACCGGATACCTGTCCGCCTTTCTCCCTTCGGGAAGCGTG |
| | | GCGCTTTCTCATAGCTCACGCTGTAGGTATCTCAGTTCGGTGTAGGTCG |
| | | TTCGCTCCAAGCTGGGCTGTGTGCACGAACCCCCCGTTCAGCCCGACCG |
| | | CTGCGCCTTATCCGGTAACTATCGTCTTGAGTCCAACCCGGTAAGACAC |
| | | GACTTATCGCCACTGGCAGCAGCCACTGGTAACAGGATTAGCAGAGCGA |
| | | GGTATGTAGGCGGTGCTACAGAGTTCTTGAAGTGGTGGCCTAACTACGG |
| | | CTACACTAGAAGAACAGTATTTGGTATCTGCGCTCTGCTGAAGCCAGTT |
| | | ACCTTCGGAAAAAGAGTTGGTAGCTCTTGATCCGGCAAACAAACCACCG |
| | | CTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCT |
| | | CAGTGGAACGAAAACTCACGTTAAGGGATTTTGGTCATGAGATTATCAA |
| | | AAAGGATCTTCACCTAGATCCTTTTAAATTAAAAATGAAGTTTTAAATC |
| | | AATCTAAAGTATATATGAGTAAACTTGGTCTGACAGTTACCAATGCTTA |
| | | ATCAGTGAGGCACCTATCTCAGCGATCTGTCTATTTCGTTCATCCATAG |
| | | TTGCCTGACTCCCCGTCGTGTAGATAACTACGATACGGGAGGGCTTACC |
| | | ATCTGGCCCCAGTGCTGCAATGATACCGCGAGAACCACGCTCACCGGCT |
| | | CCAGATTTATCAGCAATAAACCAGCCAGCCGGAAGGGCCGAGCGCAGAA |
| | | GTGGTCCTGCAACTTTATCCGCCTCCATCCAGTCTATTAATTGTTGCCG |
| | | GGAAGCTAGAGTAAGTAGTTCGCCAGTTAATAGTTTGCGCAACGTTGTT |
| | | GCCATTGCTACAGGCATCGTGGTGTCACGCTCGTCGTTTGGTATGGCTT |
| | | CATTCAGCTCCGGTTCCCAACGATCAAGGCGAGTTACATGATCCCCCAT |
| | | GTTGTGCAAAAAAGCGGTTAGCTCCTTCGGTCCTCCGATCGTTGTCAGA |
| | | AGTAAGTTGGCCGCAGTGTTATCACTCATGGTTATGGCAGCACTGCATA |
| | | ATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGC |
| | | TCTTGCCCGGCGTCAATACGGGATAATACCGCGCCACATAGCAGAACTT |
| | | TAAAAGTGCTCATCATTGGAAAACGTTCTTCGGGGCGAAAACTCTCAAG |
| | | GATCTTACCGCTGTTGAGATCCAGTTCGATGTAACCCACTCGTGCACCC |
| | | AACTGATCTTCAGCATCTTTTACTTTCACCAGCGTTTCTGGGTGAGCAA |
| | | AAACAGGAAGGCAAAATGCCGCAAAAAAGGGAATAAGGGCGACACGGAA |
| | | ATGTTGAATACTCATACTCTTCCTTTTTCAATATTATTGAAGCATTTAT |
| | | CAGGGTTATTGTCTCATGAGCGGATACATATTTGAATGTATTTAGAAAA |
| | | ATAAACAAATAGGGGTTCCGCGCACATTTCCCCGAAAAGTGCCAC |
| 164 | Compound A13 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATATAGTGAGTCGTATTAACGTACC |
| | | AACAAGAAGGAAAGTAATGGACCAGTACTTGACTGGTCCATTACTTTCC |
| | | TTCTTTATCTTAGAGGCATATCCCTACGTACCAACAAGGTCCTCACTTT |
| | | AATCCTCTAACTTGTAGAGGATTAAAGTGAGGACCTTTATCTTAGAGGC |
| | | ATATCCCTACGTACCAACAAGGAGACTTGGGCAATGTGACTACTTGAGT |
| | | CACATTGCCCAAGTCTCCTTTATCTTAGAGGCATATCCCTGCCACCATG |
| | | GGAGTGCATGAATGTCCTGCTTGGCTGTGGCTGCTGCTGAGCCTGCTGT |
| | | CTCTGCCTCTGGGACTGCCTGTTCTTGGAGCCCCTCCTAGACTGATCTG |
| | | CGACAGCAGAGTGCTGGAAAGATACCTGCTGGAAGCCAAAGAGGCCGAG |
| | | AACATCACCACAGGCTGTGCCGAGCACTGCAGCCTGAACGAGAATATCA |
| | | CCGTGCCTGACACCAAAGTGAACTTCTACGCCTGGAAGCGGATGGAAGT |
| | | GGGCCAGCAGGCTGTGGAAGTTTGGCAAGGACTGGCCCTGCTGAGCGAA |
| | | GCTGTTCTGAGAGGACAGGCTCTGCTGGTCAACAGCTCTCAGCCTTGGG |
| | | AACCTCTGCAACTGCACGTGGACAAGGCCGTGTCTGGCCTGAGAAGCCT |
| | | GACCACACTGCTGAGAGCACTGGGAGCCCAGAAAGAGGCCATCTCTCCA |
| | | CCTGATGCTGCCTCTGCTGCCCCTCTGAGAACCATCACCGCCGACACCT |
| | | TCAGAAAGCTGTTCCGGGTGTACAGCAACTTCCTGCGGGGCAAGCTGAA |
| | | GCTGTACACAGGCGAGGCTTGCAGAACCGGCGACAGATAATTTATCTTA |
| | | GAGGCATATCCCTCTGGGCCTCATGGGCCTTCCGCTCACTGCCCGCTTT |
| | | CCAGTCGGGAAACCTGTCGTGCCAGCTGCATTAACATGGTCATAGCTGT |
| | | TTCCTTGCGTATTGGGCGCTCTCCGCTTCCTCGCTCACTGACTCGCTGC |
| | | GCTCGGTCGTTCGGGTAAAGCCTGGGGTGCCTAATGAGCAAAAGGCCAG |
| | | CAAAAGGCCAGGAACCGTAAAAAGGCCGCGTTGCTGGCGTTTTTCCATA |
| | | GGCTCCGCCCCCCTGACGAGCATCACAAAAATCGACGCTCAAGTCAGAG |
| | | GTGGCGAAACCCGACAGGACTATAAAGATACCAGGCGTTTCCCCCTGGA |
| | | AGCTCCCTCGTGCGCTCTCCTGTTCCGACCCTGCCGCTTACCGGATACC |
| | | TGTCCGCCTTTCTCCCTTCGGGAAGCGTGGCGCTTTCTCATAGCTCACG |
| | | CTGTAGGTATCTCAGTTCGGTGTAGGTCGTTCGCTCCAAGCTGGGCTGT |
| | | GTGCACGAACCCCCCGTTCAGCCCGACCGCTGCGCCTTATCCGGTAACT |
| | | ATCGTCTTGAGTCCAACCCGGTAAGACACGACTTATCGCCACTGGCAGC |
| | | AGCCACTGGTAACAGGATTAGCAGAGCGAGGTATGTAGGCGGTGCTACA |
| | | GAGTTCTTGAAGTGGTGGCCTAACTACGGCTACACTAGAAGAACAGTAT |
| | | TTGGTATCTGCGCTCTGCTGAAGCCAGTTACCTTCGGAAAAAGAGTTGG |
| | | TAGCTCTTGATCCGGCAAACAAACCACCGCTGGTAGCGGTGGTTTTTTT |
| | | GTTTGCAAGCAGCAGATTACGCGCAGAAAAAAAGGATCTCAAGAAGATC |
| | | CTTTGATCTTTTCTACGGGGTCTGACGCTCAGTGGAACGAAAACTCACG |
| | | TTAAGGGATTTTGGTCATGAGATTATCAAAAAGGATCTTCACCTAGATC |
| | | CTTTTAAATTAAAAATGAAGTTTTAAATCAATCTAAAGTATATATGAGT |
| | | AAACTTGGTCTGACAGTTACCAATGCTTAATCAGTGAGGCACCTATCTC |
| | | AGCGATCTGTCTATTTCGTTCATCCATAGTTGCCTGACTCCCCGTCGTG |
| | | TAGATAACTACGATACGGGAGGGCTTACCATCTGGCCCCAGTGCTGCAA |
| | | TGATACCGCGAGAACCACGCTCACCGGCTCCAGATTTATCAGCAATAAA |
| | | CCAGCCAGCCGGAAGGGCCGAGCGCAGAAGTGGTCCTGCAACTTTATCC |
| | | GCCTCCATCCAGTCTATTAATTGTTGCCGGGAAGCTAGAGTAAGTAGTT |
| | | CGCCAGTTAATAGTTTGCGCAACGTTGTTGCCATTGCTACAGGCATCGT |
| | | GGTGTCACGCTCGTCGTTTGGTATGGCTTCATTCAGCTCCGGTTCCCAA |
| | | CGATCAAGGCGAGTTACATGATCCCCCATGTTGTGCAAAAAAGCGGTTA |
| | | GCTCCTTCGGTCCTCCGATCGTTGTCAGAAGTAAGTTGGCCGCAGTGTT |
| | | ATCACTCATGGTTATGGCAGCACTGCATAATTCTCTTACTGTCATGCCA |
| | | TCCGTAAGATGCTTTTCTGTGACTGGTGAGTACTCAACCAAGTCATTCT |
| | | GAGAATAGTGTATGCGGCGACCGAGTTGCTCTTGCCCGGCGTCAATACG |
| | | GGATAATACCGCGCCACATAGCAGAACTTTAAAAGTGCTCATCATTGGA |
| | | AAACGTTCTTCGGGGCGAAAACTCTCAAGGATCTTACCGCTGTTGAGAT |
| | | CCAGTTCGATGTAACCCACTCGTGCACCCAACTGATCTTCAGCATCTTT |
| | | TACTTTCACCAGCGTTTCTGGGTGAGCAAAAACAGGAAGGCAAAATGCC |
| | | GCAAAAAAGGGAATAAGGGCGACACGGAAATGTTGAATACTCATACTCT |
| | | TCCTTTTTCAATATTATTGAAGCATTTATCAGGGTTATTGTCTCATGAG |
| | | CGGATACATATTTGAATGTATTTAGAAAAATAAACAAATAGGGGTTCCG |
| | | CGCACATTTCCCCGAAAAGTGCCAC |
| 165 | Compound A14 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATAATAGTGAGTCGTATTAACGTAC |
| | | CAACAAGAAAGATGATAAGCCCACTCTACTTGAGAGTGGGCTTATCATC |
| | | TTTCTTTATCTTAGAGGCATATCCCTACGTACCAACAAGGTGATGTCTG |
| | | GTCCATATGAACTTGTCATATGGACCAGACATCACCTTTATCTTAGAGG |
| | | CATATCCCTACGTACCAACAAGATGATAAGCCCACTCTAACTTGTAGAG |
| | | TGGGCTTATCATCTTTATCTTAGAGGCATATCCCTGCCACCATGGGCAA |
| | | GATTAGCAGCCTGCCTACACAGCTGTTCAAGTGCTGCTTCTGCGACTTC |
| | | CTGAAAGTGAAGATGCACACCATGAGCAGCAGCCACCTGTTCTATCTGG |
| | | CCCTGTGCCTGCTGACCTTTACCAGCTCTGCTACCGCCGGACCTGAGAC |
| | | ACTTTGTGGCGCTGAACTGGTGGACGCCCTGCAGTTTGTGTGTGGCGAC |
| | | AGAGGCTTCTACTTCAACAAGCCCACAGGCTACGGCAGCAGCTCTAGAA |
| | | GGGCTCCTCAGACCGGAATCGTGGACGAGTGCTGTTTCAGAAGCTGCGA |
| | | CCTGCGGCGGCTGGAAATGTATTGTGCCCCTCTGAAGCCTGCCAAGAGC |
| | | GCCTAATTTATCTTAGAGGCATATCCCTCTGGGCCTCATGGGCCTTCCG |
| | | CTCACTGCCCGCTTTCCAGTCGGGAAACCTGTCGTGCCAGCTGCATTAA |
| | | CATGGTCATAGCTGTTTCCTTGCGTATTGGGCGCTCTCCGCTTCCTCGC |
| | | TCACTGACTCGCTGCGCTCGGTCGTTCGGGTAAAGCCTGGGGTGCCTAA |
| | | TGAGCAAAAGGCCAGCAAAAGGCCAGGAACCGTAAAAAGGCCGCGTTGC |
| | | TGGCGTTTTTCCATAGGCTCCGCCCCCCTGACGAGCATCACAAAAATCG |
| | | ACGCTCAAGTCAGAGGTGGCGAAACCCGACAGGACTATAAAGATACCAG |
| | | GCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCT |
| | | TTCTCATAGCTCACGCTGTAGGTATCTCAGTTCGGTGTAGGTCGTTCGC |
| | | TCCAAGCTGGGCTGTGTGCACGAACCCCCCGTTCAGCCCGACCGCTGCG |
| | | CCTTATCCGGTAACTATCGTCTTGAGTCCAACCCGGTAAGACACGACTT |
| | | ATCGCCACTGGCAGCAGCCACTGGTAACAGGATTAGCAGAGCGAGGTAT |
| | | GTAGGCGGTGCTACAGAGTTCTTGAAGTGGTGGCCTAACTACGGCTACA |
| | | CTAGAAGAACAGTATTTGGTATCTGCGCTCTGCTGAAGCCAGTTACCTT |
| | | CGGAAAAAGAGTTGGTAGCTCTTGATCCGGCAAACAAACCACCGCTGGT |
| | | AGCGGTGGTTTTTTTGTTTGCAAGCAGCAGATTACGCGCAGAAAAAAAG |
| | | GATCTCAAGAAGATCCTTTGATCTTTTCTACGGGGTCTGACGCTCAGTG |
| | | GAACGAAAACTCACGTTAAGGGATTTTGGTCATGAGATTATCAAAAAGG |
| | | ATCTTCACCTAGATCCTTTTAAATTAAAAATGAAGTTTTAAATCAATCT |
| | | AAAGTATATATGAGTAAACTTGGTCTGACAGTTACCAATGCTTAATCAG |
| | | TGAGGCACCTATCTCAGCGATCTGTCTATTTCGTTCATCCATAGTTGCC |
| | | TGACTCCCCGTCGTGTAGATAACTACGATACGGGAGGGCTTACCATCTG |
| | | GCCCCAGTGCTGCAATGATACCGCGAGAACCACGCTCACCGGCTCCAGA |
| | | TTTATCAGCAATAAACCAGCCAGCCGGAAGGGCCGAGCGCAGAAGTGGT |
| | | CCTGCAACTTTATCCGCCTCCATCCAGTCTATTAATTGTTGCCGGGAAG |
| | | CTAGAGTAAGTAGTTCGCCAGTTAATAGTTTGCGCAACGTTGTTGCCAT |
| | | TGCTACAGGCATCGTGGTGTCACGCTCGTCGTTTGGTATGGCTTCATTC |
| | | AGCTCCGGTTCCCAACGATCAAGGCGAGTTACATGATCCCCCATGTTGT |
| | | GCAAAAAAGCGGTTAGCTCCTTCGGTCCTCCGATCGTTGTCAGAAGTAA |
| | | GTTGGCCGCAGTGTTATCACTCATGGTTATGGCAGCACTGCATAATTCT |
| | | CTTACTGTCATGCCATCCGTAAGATGCTTTTCTGTGACTGGTGAGTACT |
| | | CAACCAAGTCATTCTGAGAATAGTGTATGCGGCGACCGAGTTGCTCTTG |
| | | CCCGGCGTCAATACGGGATAATACCGCGCCACATAGCAGAACTTTAAAA |
| | | GTGCTCATCATTGGAAAACGTTCTTCGGGGCGAAAACTCTCAAGGATCT |
| | | TACCGCTGTTGAGATCCAGTTCGATGTAACCCACTCGTGCACCCAACTG |
| | | ATCTTCAGCATCTTTTACTTTCACCAGCGTTTCTGGGTGAGCAAAAACA |
| | | GGAAGGCAAAATGCCGCAAAAAAGGGAATAAGGGCGACACGGAAATGTT |
| | | GAATACTCATACTCTTCCTTTTTCAATATTATTGAAGCATTTATCAGGG |
| | | TTATTGTCTCATGAGCGGATACATATTTGAATGTATTTAGAAAAATAAA |
| | | CAAATAGGGGTTCCGCGCACATTTCCCCGAAAAGTGCCAC |
| 166 | Compound A15 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATGCCACCATGGGCAAGATTAGCAG |
| | | CCTGCCTACACAGCTGTTCAAGTGCTGCTTCTGCGACTTCCTGAAAGTG |
| | | AAGATGCACACCATGAGCAGCAGCCACCTGTTCTATCTGGCCCTGTGCC |
| | | TGCTGACCTTTACCAGCTCTGCTACCGCCGGACCTGAGACACTTTGTGG |
| | | CGCTGAACTGGTGGACGCCCTGCAGTTTGTGTGTGGCGACAGAGGCTTC |
| | | TACTTCAACAAGCCCACAGGCTACGGCAGCAGCTCTAGAAGGGCTCCTC |
| | | AGACCGGAATCGTGGACGAGTGCTGTTTCAGAAGCTGCGACCTGCGGCG |
| | | GCTGGAAATGTATTGTGCCCCTCTGAAGCCTGCCAAGAGCGCCTAAATA |
| | | GTGAGTCGTATTAACGTACCAACAAGAAAGATGATAAGCCCACTCTACT |
| | | TGAGAGTGGGCTTATCATCTTTCTTTATCTTAGAGGCATATCCCTACGT |
| | | ACCAACAAGGTGATGTCTGGTCCATATGAACTTGTCATATGGACCAGAC |
| | | ATCACCTTTATCTTAGAGGCATATCCCTACGTACCAACAAGATGATAAG |
| | | CCCACTCTAACTTGTAGAGTGGGCTTATCATCTTTATCTTAGAGGCATA |
| | | TCCCTTTTATCTTAGAGGCATATCCCTCTGGGCCTCATGGGCCTTCCGC |
| | | TCACTGCCCGCTTTCCAGTCGGGAAACCTGTCGTGCCAGCTGCATTAAC |
| | | ATGGTCATAGCTGTTTCCTTGCGTATTGGGCGCTCTCCGCTTCCTCGCT |
| | | CACTGACTCGCTGCGCTCGGTCGTTCGGGTAAAGCCTGGGGTGCCTAAT |
| | | GAGCAAAAGGCCAGCAAAAGGCCAGGAACCGTAAAAAGGCCGCGTTGCT |
| | | GGCGTTTTTCCATAGGCTCCGCCCCCCTGACGAGCATCACAAAAATCGA |
| | | CGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCC |
| | | GCTTACCGGATACCTGTCCGCCTTTCTCCCTTCGGGAAGCGTGGCGCTT |
| | | TCTCATAGCTCACGCTGTAGGTATCTCAGTTCGGTGTAGGTCGTTCGCT |
| | | CCAAGCTGGGCTGTGTGCACGAACCCCCCGTTCAGCCCGACCGCTGCGC |
| | | CTTATCCGGTAACTATCGTCTTGAGTCCAACCCGGTAAGACACGACTTA |
| | | TCGCCACTGGCAGCAGCCACTGGTAACAGGATTAGCAGAGCGAGGTATG |
| | | TAGGCGGTGCTACAGAGTTCTTGAAGTGGTGGCCTAACTACGGCTACAC |
| | | TAGAAGAACAGTATTTGGTATCTGCGCTCTGCTGAAGCCAGTTACCTTC |
| | | GGAAAAAGAGTTGGTAGCTCTTGATCCGGCAAACAAACCACCGCTGGTA |
| | | GCGGTGGTTTTTTTGTTTGCAAGCAGCAGATTACGCGCAGAAAAAAAGG |
| | | ATCTCAAGAAGATCCTTTGATCTTTTCTACGGGGTCTGACGCTCAGTGG |
| | | AACGAAAACTCACGTTAAGGGATTTTGGTCATGAGATTATCAAAAAGGA |
| | | TCTTCACCTAGATCCTTTTAAATTAAAAATGAAGTTTTAAATCAATCTA |
| | | AAGTATATATGAGTAAACTTGGTCTGACAGTTACCAATGCTTAATCAGT |
| | | GAGGCACCTATCTCAGCGATCTGTCTATTTCGTTCATCCATAGTTGCCT |
| | | GACTCCCCGTCGTGTAGATAACTACGATACGGGAGGGCTTACCATCTGG |
| | | CCCCAGTGCTGCAATGATACCGCGAGAACCACGCTCACCGGCTCCAGAT |
| | | TTATCAGCAATAAACCAGCCAGCCGGAAGGGCCGAGCGCAGAAGTGGTC |
| | | CTGCAACTTTATCCGCCTCCATCCAGTCTATTAATTGTTGCCGGGAAGC |
| | | TAGAGTAAGTAGTTCGCCAGTTAATAGTTTGCGCAACGTTGTTGCCATT |
| | | GCTACAGGCATCGTGGTGTCACGCTCGTCGTTTGGTATGGCTTCATTCA |
| | | GCTCCGGTTCCCAACGATCAAGGCGAGTTACATGATCCCCCATGTTGTG |
| | | CAAAAAAGCGGTTAGCTCCTTCGGTCCTCCGATCGTTGTCAGAAGTAAG |
| | | TTGGCCGCAGTGTTATCACTCATGGTTATGGCAGCACTGCATAATTCTC |
| | | TTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGC |
| | | CCGGCGTCAATACGGGATAATACCGCGCCACATAGCAGAACTTTAAAAG |
| | | TGCTCATCATTGGAAAACGTTCTTCGGGGCGAAAACTCTCAAGGATCTT |
| | | ACCGCTGTTGAGATCCAGTTCGATGTAACCCACTCGTGCACCCAACTGA |
| | | TCTTCAGCATCTTTTACTTTCACCAGCGTTTCTGGGTGAGCAAAAACAG |
| | | GAAGGCAAAATGCCGCAAAAAAGGGAATAAGGGCGACACGGAAATGTTG |
| | | AATACTCATACTCTTCCTTTTTCAATATTATTGAAGCATTTATCAGGGT |
| | | TATTGTCTCATGAGCGGATACATATTTGAATGTATTTAGAAAAATAAAC |
| | | AAATAGGGGTTCCGCGCACATTTCCCCGAAAAGTGCCAC |
| 159 | Compound B18 | CTAAATTGTAAGCGTTAATATTTTGTTAAAATTCGCGTTAAATTTTTGT |
| | in pMA-RQ | TAAATCAGCTCATTTTTTAACCAATAGGCCGAAATCGGCAAAATCCCTT |
| | | ATAAATCAAAAGAATAGACCGAGATAGGGTTGAGTGGCCGCTACAGGGC |
| | | GCTCCCATTCGCCATTCAGGCTGCGCAACTGTTGGGAAGGGCGTTTCGG |
| | | TGCGGGCCTCTTCGCTATTACGCCAGCTGGCGAAAGGGGGATGTGCTGC |
| | | AAGGCGATTAAGTTGGGTAACGCCAGGGTTTTCCCAGTCACGACGTTGT |
| | | AAAACGACGGCCAGTGAGCGCGACGTAATACGACTCACTATAGGGCGAA |
| | | TTGGCGGAAGGCCGTCAAGGCCGCATGCCACCATGTCTAGCAGCTCTTG |
| | | GCTGCTGCTGTCTCTGGTGGCTGTGACAGCCGCTCAGAGCACCATTGAG |
| | | GAACAGGCCAAGACCTTCCTGGACAAGTTCAACCACGAGGCCGAGGACC |
| | | TGTTCTACCAGTCTAGCCTGGCCAGCTGGAACTACAACACCAACATCAC |
| | | CGAAGAGAACGTGCAGAACATGAACAACGCCGGCGACAAGTGGAGCGCC |
| | | TTCCTGAAAGAGCAGAGCACACTGGCCGAGATGTACCCTCTGCAAGAGA |
| | | TCCAGAACCTGACCGTGAAGCTCCAGCTGCAGGCCCTCCAGCAGAATGG |
| | | AAGCTCTGTGCTGAGCGAGGACAAGAGCAAGCGGCTGAACACCATCCTG |
| | | AATACCATGAGCACCATCTACAGCACCGGCAAAGTGTGCAACCCCGACA |
| | | ATCCCCAAGAGTGCCTGCTGCTGGAACCCGGCCTGAATGAGATCATGGC |
| | | CAACAGCCTGGACTACAACGAGAGACTGTGGGCCTGGGAGTCTTGGAGA |
| | | AGCGAAGTGGGAAAGCAGCTGCGGCCCCTGTACGAGGAATACGTGGTGC |
| | | TGAAGAACGAGATGGCCAGAGCCAACCACTACGAGGACTACGGCGACTA |
| | | TTGGAGAGGCGACTACGAAGTGAATGGCGTGGACGGCTACGACTACAGC |
| | | AGAGGCCAGCTGATCGAGGAGGTGGAACACACCTTCGAGGAAATCAAGC |
| | | CTCTGTACGAGCATCTGCACGCCTACGTGCGGGCCAAGCTGATGAATGC |
| | | TTACCCCAGCTACATCAGCCCCATCGGCTGTCTGCCTGCTCATCTGCTG |
| | | GGAGACATGTGGGGCAGATTCTGGACCAACCTGTACAGCCTGACAGTGC |
| | | CCTTCGGCCAGAAACCTAACATCGACGTGACCGACGCCATGGTGGATCA |
| | | GGCTTGGGATGCCCAGCGGATCTTCAAAGAGGCCGAGAAGTTCTTCGTG |
| | | TCCGTGGGCCTGCCTAATATGACCCAAGGCTTCTGGGAGAACTCCATGC |
| | | TGACAGACCCCGGCAATGTGCAGAAAGCCGTGTGTCATCCTACCGCCTG |
| | | GGATCTCGGCAAGGGCGACTTCAGAATCCTGATGTGCACCAAAGTGACG |
| | | ATGGACGACTTCCTGACAGCCCACCACGAGATGGGCCACATCCAGTACG |
| | | ATATGGCCTACGCCGCTCAGCCCTTCCTGCTGAGAAATGGCGCCAATGA |
| | | GGGCTTCCACGAAGCCGTGGGAGAGATCATGAGCCTGTCTGCCGCCACA |
| | | CCTAAGCACCTGAAGTCTATCGGACTGCTGAGCCCCGACTTCCAAGAGG |
| | | ACAACGAGACAGAGATCAACTTCCTGCTCAAGCAGGCCCTGACCATCGT |
| | | GGGCACACTGCCCTTTACCTACATGCTGGAAAAGTGGCGGTGGATGGTC |
| | | TTTAAGGGCGAGATCCCCAAGGACCAGTGGATGAAGAAATGGTGGGAGA |
| | | TGAAGCGCGAGATCGTGGGCGTTGTGGAACCTGTGCCTCACGACGAGAC |
| | | ATACTGCGATCCTGCCAGCCTGTTTCACGTGTCCAACGACTACTCCTTC |
| | | ATCCGGTACTACACCCGGACACTGTACCAGTTCCAGTTTCAAGAGGCTC |
| | | TGTGCCAGGCCGCCAAGCACGAAGGACCTCTGCACAAGTGCGACATCAG |
| | | CAACTCTACAGAGGCCGGACAGAAACTGTTCAACATGCTGCGGCTGGGC |
| | | AAGAGCGAGCCTTGGACACTGGCTCTGGAAAATGTCGTGGGCGCCAAGA |
| | | ATATGAACGTGCGGCCACTGCTGAACTACTTCGAGCCCCTGTTCACCTG |
| | | GCTGAAGGACCAGAACAAGAACAGCTTCGTCGGCTGGTCCACCGATTGG |
| | | AGCCCTTACGCCGACCAGAGCATCAAAGTGCGGATCAGCCTGAAAAGCG |
| | | CCCTGGGCGATAAGGCCTATGAGTGGAACGACAATGAGATGTACCTGTT |
| | | CCGGTCCAGCGTGGCCTATGCTATGCGGCAGTACTTTCTGAAAGTCAAG |
| | | AACCAGATGATCCTGTTCGGCGAAGAGGATGTGCGCGTGGCCAACCTGA |
| | | AGCCTCGGATCAGCTTCAACTTCTTCGTGACTGCCCCTAAGAACGTGTC |
| | | CGACATCATCCCCAGAACCGAGGTGGAAAAGGCCATCAGAATGAGCAGA |
| | | AGCCGGATCAACGACGCCTTCCGGCTGAACGACAACTCCCTGGAATTCC |
| | | TGGGCATTCAGCCCACACTGGGCCCTCCAAATCAGCCTCCTGTGTCCTA |
| | | AATAGTGAGTCGTATTAACGTACCAACAAGTGTGACCGAAAGGTAAGAT |
| | | GACTTGCATCTTACCTTTCGGTCACACTTTATCTTAGAGGCATATCCCT |
| | | ACGTACCAACAAGAGGTGATGAAGTCAGACAAAACTTGTTTGTCTGACT |
| | | TCATCACCTCTTTATCTTAGAGGCATATCCCTACGTACCAACAAGCAAC |
| | | TGAGGGAGCCTTGAATACTTGATTCAAGGCTCCCTCAGTTGCTTTATCT |
| | | TAGAGGCATATCCCTTTTATCTTAGAGGCATATCCCTCTGGGCCTCATG |
| | | GGCCTTCCGCTCACTGCCCGCTTTCCAGTCGGGAAACCTGTCGTGCCAG |
| | | CTGCATTAACATGGTCATAGCTGTTTCCTTGCGTATTGGGCGCTCTCCG |
| | | CTTCCTCGCTCACTGACTCGCTGCGCTCGGTCGTTCGGGTAAAGCCTGG |
| | | GGTGCCTAATGAGCAAAAGGCCAGCAAAAGGCCAGGAACCGTAAAAAGG |
| | | CCGCGTTGCTGGCGTTTTTCCATAGGCTCCGCCCCCCTGACGAGCATCA |
| | | CAAAAATCGACGCTCAAGTCAGAGGTGGCGAAACCCGACAGGACTATAA |
| | | AGATACCAGGCGTTTCCCCCTGGAAGCTCCCTCGTGCGCTCTCCTGTTC |
| | | CGACCCTGCCGCTTACCGGATACCTGTCCGCCTTTCTCCCTTCGGGAAG |
| | | CGTGGCGCTTTCTCATAGCTCACGCTGTAGGTATCTCAGTTCGGTGTAG |
| | | GTCGTTCGCTCCAAGCTGGGCTGTGTGCACGAACCCCCCGTTCAGCCCG |
| | | ACCGCTGCGCCTTATCCGGTAACTATCGTCTTGAGTCCAACCCGGTAAG |
| | | ACACGACTTATCGCCACTGGCAGCAGCCACTGGTAACAGGATTAGCAGA |
| | | GCGAGGTATGTAGGCGGTGCTACAGAGTTCTTGAAGTGGTGGCCTAACT |
| | | ACGGCTACACTAGAAGAACAGTATTTGGTATCTGCGCTCTGCTGAAGCC |
| | | AGTTACCTTCGGAAAAAGAGTTGGTAGCTCTTGATCCGGCAAACAAACC |
| | | ACCGCTGGTAGCGGTGGTTTTTTTGTTTGCAAGCAGCAGATTACGCGCA |
| | | GAAAAAAAGGATCTCAAGAAGATCCTTTGATCTTTTCTACGGGGTCTGA |
| | | CGCTCAGTGGAACGAAAACTCACGTTAAGGGATTTTGGTCATGAGATTA |
| | | TCAAAAAGGATCTTCACCTAGATCCTTTTAAATTAAAAATGAAGTTTTA |
| | | AATCAATCTAAAGTATATATGAGTAAACTTGGTCTGACAGTTACCAATG |
| | | CTTAATCAGTGAGGCACCTATCTCAGCGATCTGTCTATTTCGTTCATCC |
| | | ATAGTTGCCTGACTCCCCGTCGTGTAGATAACTACGATACGGGAGGGCT |
| | | TACCATCTGGCCCCAGTGCTGCAATGATACCGCGAGAACCACGCTCACC |
| | | GGCTCCAGATTTATCAGCAATAAACCAGCCAGCCGGAAGGGCCGAGCGC |
| | | AGAAGTGGTCCTGCAACTTTATCCGCCTCCATCCAGTCTATTAATTGTT |
| | | GCCGGGAAGCTAGAGTAAGTAGTTCGCCAGTTAATAGTTTGCGCAACGT |
| | | TGTTGCCATTGCTACAGGCATCGTGGTGTCACGCTCGTCGTTTGGTATG |
| | | GCTTCATTCAGCTCCGGTTCCCAACGATCAAGGCGAGTTACATGATCCC |
| | | CCATGTTGTGCAAAAAAGCGGTTAGCTCCTTCGGTCCTCCGATCGTTGT |
| | | CAGAAGTAAGTTGGCCGCAGTGTTATCACTCATGGTTATGGCAGCACTG |
| | | CATAATTCTCTTACTGTCATGCCATCCGTAAGATGCTTTTCTGTGACTG |
| | | GTGAGTACTCAACCAAGTCATTCTGAGAATAGTGTATGCGGCGACCGAG |
| | | TTGCTCTTGCCCGGCGTCAATACGGGATAATACCGCGCCACATAGCAGA |
| | | ACTTTAAAAGTGCTCATCATTGGAAAACGTTCTTCGGGGCGAAAACTCT |
| | | CAAGGATCTTACCGCTGTTGAGATCCAGTTCGATGTAACCCACTCGTGC |
| | | ACCCAACTGATCTTCAGCATCTTTTACTTTCACCAGCGTTTCTGGGTGA |
| | | GCAAAAACAGGAAGGCAAAATGCCGCAAAAAAGGGAATAAGGGCGAGAC |
| | | GGAAATGTTGAATACTCATACTCTTCCTTTTTCAATATTATTGAAGCAT |
| | | TTATCAGGGTTATTGTCTCATGAGCGGATACATATTTGAATGTATTTAG |
| | | AAAAATAAACAAATAGGGGTTCCGCGCACATTTCCCCGAAAAGTGCCAC |

Bold and underline = compound sequence

In Vitro Transcription of RNA Constructs and Data Analysis

Details of in vitro transcription are provided in Example 2. Using in vitro transcription, Compound A9 and Compound A10 were generated at 50-200 μg range and were tested for endogenous TNF-α downregulation and IL-4 expression in THP-1 cells where endogenous TNF-α expression was stimulated by the treatment with LPS and R848 (Example 17). Likewise, Compound A9 and Compound A10 were tested for TNF-α downregulation and IL-4 expression in overexpression models of HEK-293 cells where TNF-α was overexpressed using TNF-α encoding mRNA (Example 18).

Further, Compound A11 was generated at 50-200 μg range and were tested for endogenous ALK2 downregulation and IGF-1 expression in A549 cells (Example 19). In addition, Compound A12 and Compound A13 were generated at 50-200 μg range and were tested for endogenous SOD1 downregulation along with expression of IGF-1 and Erythropoietin (EPO), respectively, in IMR32 cells (Example 20). Compounds A15 and A16 were generated at 50-200 μg range and were tested for the expression of IGF-1 and IL-1 beta downregulation in an overexpression model using HEK293 cells. IL-1-beta protein was overexpressed using IL-1 beta encoding mRNA (Example 21).

Compound B18 was generated at 50-200 μg range and was tested for the expression of soluble ACE2 receptor and downregulation of eGFP tagged SARS CoV-2 Nucleocapsid protein in an overexpression model using A549 cells where eGFP tag-SARS CoV-2 Nucleocapsid protein was overexpressed from a pCDNA3⁺ vector (Example 22).

Data were analyzed using GraphPad Prism 8 (San Diego, USA). For the estimation of the protein (IGF-1, IL-4, IL-1 beta, ALK2, SOD1, EPO, and TNF-α) levels using ELISA in the standard or the sample, the mean absorbance value of the blank was subtracted from the mean absorbance of the standards or the samples. A standard curve was generated and plotted using a four parameters nonlinear regression according to manufacturer's protocol. To determine the concentration of proteins (IGF-1, IL-4, IL-1 beta, ALK2, SOD1, EPO, and TNF-α) in each sample, the concentration of the protein was interpolated from the standard curve. The final protein concentration of the sample was calculated by multiplication with the dilution factor.

Statistical analyses were made using a Student's t-test or one way ANOVA followed by Dunnet's multiple comparing test related to control. The percent of GFP positive cells was calculated using SoftMax Pro tool in Example 22. Relative quantification of remaining target mRNA post treatment with compounds was carried out using the 2^{-ΔΔct} method between study groups. The level of significance was set to a P-value of <0.05. Determination of the molecular weight of Compound A11 was performed as below. The molecular weight of Compound A11 was calculated based on its mRNA sequence by multiplying the number of each base by the molecular weight of the base (e.g., A: 347.2 g/mol; C 323.2 g/mol; G 363.2 g/mol; N1-UTP:338.2 g/mol). The compound molecular weight was determined by adding the obtained weight totals for each base to the ARCA molecular weight of 817.4 g/mol. The molecular weight of the construct was used to convert the amount of transfected mRNA in the well to nM concentration.

### Example 17: Endogenous TNF-α Expression Model in THP-1 Cells

Compound A9 and Compound A10 were assayed for their ability to downregulate TNF-α expression, and overexpress IL-4, in THP-1 cells. For the endogenous secretion of TNF-α in THP-1 cells, THP-1 cells were stimulated with E. coli-derived lipopolysaccharide (LPS-L4391; Sigma Aldrich) at 10 μg/mL final concentration with R848 (TLR7/8 agonist; Invivogen) at 1 μg/mL final concentration and incubated for 90 minutes. The induced production of TNF-α corresponds to the physiological conditions observed in psoriasis. Post stimulation, 50 μl of media was removed and replaced with the transfection complex containing specific mRNA constructs (Compounds A9 and A10) or scrambled siRNA (sc-siRNA) complexed with Lipofectamine 2000 in Opti-MEM and incubated at 37° C. in a humidified atmosphere containing 5% CO₂ for 24 hours. The sc-siRNA were used to rule out transfection related cell death (Universal siRNA, Sigma; Cat. SIC002). Post transfection, the cell culture supernatant was collected and quantified for of TNF-α (target gene to downregulate) and IL-4 (Gene of Interest to overexpress) by ELISA. The TNF-α levels in samples transfected only with TNF-α mRNA were used as controls and set to 100% and percent of TNF-α knock down was calculated.

Results

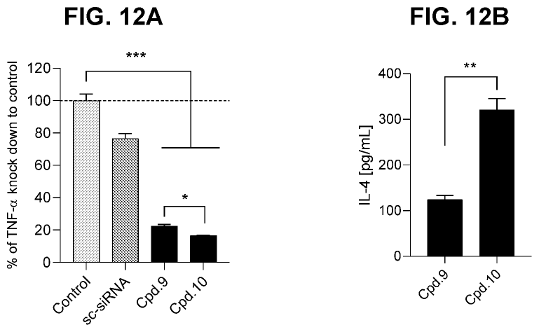

The effect of Compound A9 (comprising siRNA targeting TNF-α 5′ to the IL-4 coding sequence) and Compound A10 (comprising siRNA targeting TNF-α 3′ to the IL-4 coding sequence) on downregulation of TNF-α was evaluated in THP-1 cells stimulated with 10 μg/mL LPS and 1 μg/mL R848 to induce endogenous TNF-α secretion. The established THP-1 model mimics the physiological immune condition of psoriasis. As demonstrated in FIG. 12A, Compound A9 and Compound A10 downregulated the expression of endogenous TNF-α expression in THP-1 cells by at least approximately 80% relative to control (P<0.001). Interestingly, Compound A10 induced significantly stronger TNF-α downregulation compared to Compound A9 which has siRNA positioned upstream of (or 5′ to) IL-4 ORF (FIG. 12A; P<0.05). Compound A10 induced TNF-α downregulation of at least approximately 85% relative to control, and at approximately 5-10% greater than Compound A9. The same cell culture supernatant was measured for IL-4 expression and the data show that the expression of IL-4 by Compound A10 is 2.5-fold higher than the expression of IL-4 by Compound A9 as shown in FIG. 12B (P<0.01). This assay demonstrates that Compound A10 (TNF-α-targeting siRNA positioned at 3′ of IL-4 gene), when compared to Compound A9 (TNF-α-targeting siRNA positioned 5′ of IL-4 gene), has 5-10% greater TNF-α-targeting (downregulating) siRNA activity and 2.5-fold greater IL-4 expression (a 70% increase).

### Example 18: TNF-α Overexpression Model in HEK-293 Cells

Compound A9 and Compound A10 were assayed for their ability to downregulate TNF-α expression, and overexpress IL-4, in HEK-293 cells. To assess the simultaneous effect of TNF-α RNA interference (RNAi) and IL-4 expression, the TNF-α overexpression model was established using TNF-α mRNA transfection (600 ng/well). As described, Compound A9 comprises TNF-α-targeting siRNA 5′ of the IL-4 coding sequence (upstream of IL-4 gene) while Compound A10 comprises TNF-α-targeting siRNA 3′ of the IL-4 coding sequence (downstream of IL-4 gene). To assess the capability of Compound A9 and Compound A10 containing TNF-α targeting siRNA in TNF-α downregulation and simultaneous IL-4 expression, the cells were co-transfected with Compound A9 or Compound A10 (900 ng/well) and TNF-α mRNA (600 ng/well). Post transfection, the cells were incubated at 37° C. in a humidified atmosphere containing 5% CO₂ for 24 hours followed by quantification of TNF-α (target gene to downregulate) and IL-4 (Gene of Interest to overexpress) by ELISA in the same cell culture supernatant. The TNF-α levels in samples transfected only with TNF-α mRNA were used as controls and set to 100% and percent of TNF-α knock down was calculated.

Results

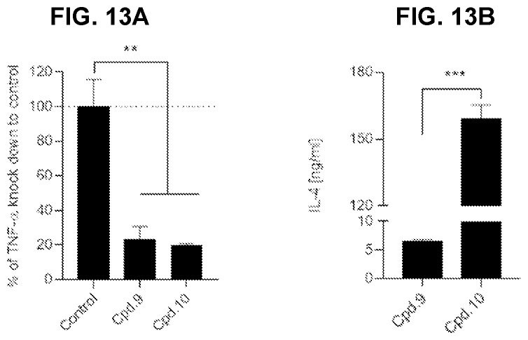

Compound A9 and Compound A10 were tested for TNF-α downregulation and IL-4 expression at the same time in HEK-293 cells (900 ng/well) with exogenously delivered TNF-α mRNA (600 ng/well). The data show 20-fold higher IL-4 expression from Compound A10 than from Compound A9, as shown in FIG. 13B (P<0.001). In the same experiment with the same cell culture supernatant, the RNA interference of Compound A9 and Compound A10 (900 ng/well) against TNF-α expression was assessed using a TNF-α overexpression construct (600 ng/well), followed by TNF-α ELISA. Both Compound A9 and Compound A10 downregulated the TNF-α level compared to untreated control up to 80% (P<0.01) as shown in FIG. 13A. The assay data shown in FIGS. 13A and 13B demonstrate that Compound A10 (which comprises TNF-α-targeting siRNA 3′ of the IL-4 coding sequence) downregulated TNF-α at least as well as Compound A9 (which comprises TNF-α-targeting siRNA 5′ of the IL-4 coding sequence), by approximately 80%. Additionally, Compound A10 induced at least a 20-fold increase in IL-4 expression relative to Compound A9.

### Example 19: Endogenous ALK2 Expression Model in A549 Cells

In Vitro Transfection of A549 Cells with Compound A11

A549 cells are typical alveolar type II (ATII) cells derived from human lung carcinoma. Since A549 cells express endogenous ALK2 RNA transcripts at a moderate level, A549 cells were used to study the effect of Compound A11 in degrading the ALK2 mRNA in parallel to measuring IGF-1 expression. The A549 cells (Sigma-Aldrich, Buchs Switzerland Cat. #6012804) were maintained on Dulbecco's Modified Eagle's medium-high glucose (DMEM, Sigma-Aldrich, Buchs Switzerland cat #D0822) supplemented with 10% FBS (Thermo Fisher Scientific, Basel, Switzerland; cat. #10500-064). To assess Compound A11 activity, the A549 cells were plated at a density of 10,000 cells/well in a regular growth medium 24 hours prior to transfection. Thereafter, cells were transfected with increasing concentration of Compound A11 (0, 0.61, 1.25, 2.54, 5.08, 10.16 and 20.33 nM, corresponding to 0, 19, 38, 75, 150, 300 or 600 ng/well, respectively) using Lipofectamine 2000 (www.invitrogen.com) following the manufacturer's instructions. 100 μl of DMEM were removed and 50 μl of Opti-MEM (www.thermofisher.com) was added to each well followed by 50 μl mRNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂, followed by IGF-1 quantification by ELISA and ALK2 mRNA by relative quantification using qPCR with primers targeting human ALK2 mRNA (Forward primer: 5′-GACGTGGAGTATGGCACTATCG-3′ and Reverse primer: 5′-CACTCCAACAGTGTAATCTGGCG-3′; SEQ ID NOs: 171 and 172, respectively) using SYBR 1-Step Cells to CT kit (Thermo Fisher Scientific, Basel, Switzerland; cat. #A25599). The human 18S rRNA was used as a reference control (Forward primer: 5′-ACCCGTTGAACCCCATTCGTGA-3′ and Reverse primer: 5′-GCCTCACTAAACCATCCAATCGG-3′; SEQ ID NOs: 173 and 174, respectively).

Results

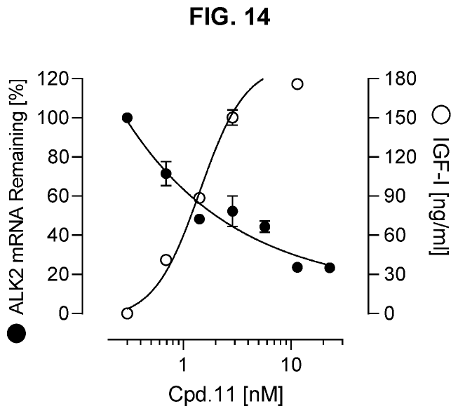

The effect of Compound A11 (comprising 3×ALK2-targeting siRNA 3′ to an IGF-1 protein coding sequence) was evaluated for ALK-2 downregulation and simultaneous IGF-1 expression in A549 cells with dose response (0.6 nM to 20.33 nM). The data demonstrate that Compound A11 expresses IGF-1 protein dose dependently, reaching a level above 150 ng/ml as shown in FIG. 14. In the same cell culture supernatant, the RNA interference of Compound A11 against remaining ALK-2 expression was assessed. As demonstrated in FIG. 14, Compound A11 downregulated the endogenous ALK2 RNA transcripts expression up to approximately 75%. This assay demonstrated that Compound A11 downregulated ALK2 expression by 75% and simultaneously expressed IGF-1 in a dose-dependent manner up to at least 150 ng/ml.

### Example 20: Endogenous SOD1 Expression Model in IMR32 Cells

Compound A12 and Compound A13 were assayed for their ability to downregulate SOD-1 expression, and overexpress IGF-1 (Compound A12) or EPO (Compound A13) in Human Caucasian Neuroblastoma (IMR32) cells. IMR32 cells (Cat #86041809, ECACC, UK) were plated at a density of 20,000 cells per well in a 96 pre-coated BRAND microtiter plate (Cat #782082) in Minimum Essential Medium Eagle (EMEM, Bioconcept Cat #1-31501-I, www.bioconcept.ch) supplemented with 10% (v/v) heat-inactivated Fetal Bovine Serum (FBS), L-Glutamine (2 mM) and Non-essential Amino acids (NEAA, 1×). Cells were grown overnight at 37° C. in a humidified atmosphere containing 5% CO₂. Cells were transfected with three doses of Compound A12 or Compound A13 (150, 300 or 900 ng/well,) constructs using JetMessenger (www.polyplus-transfection.com) following manufacturer's instructions. The scrambled siRNA (sc-siRNA) was used to rule out transfection-related cell death (Universal siRNA, Sigma; Cat. SIC002). Briefly, mRNA/JetMessenger complex was formed by mixing 0.25 μl JetMessenger reagent per 0.1 μg mRNA construct. After incubating 15 minutes at room temperature the JetMessenger complex was added as 10 μl and 5 hours after transfection medium/mRNA/JetMessenger was removed from the wells and replaced with fresh 100 μl growth medium and the plates were incubated 24 hours at 37° C. in a humidified atmosphere containing 5% CO₂. The measurement of remaining SOD1 mRNA was measured by qPCR in cell lysates 24 hours after transfection with Compound A12 and Compound A13 by relative quantification using qPCR with primers targeting human SOD1 mRNA (Forward primer: 5′-CTCACTCTCAGGAGACCATTGC-3′ and Reverse primer: 5′-CCACAAGCCAAACGACTTCCAG-3′; SEQ ID NOs: 175 and 176, respectively) using SYBR 1-Step Cells to CT kit (Thermo Fischer Scientific, Basel, Switzerland; cat. #A25599). The human 18S rRNA used as a reference control using the same primers specified in Example 19. The same cell culture supernatant was used to measure IGF-1 and EPO (Thermo Fisher Scientific, Basel, Switzerland; cat. #BMS2035) by ELISA.

Results

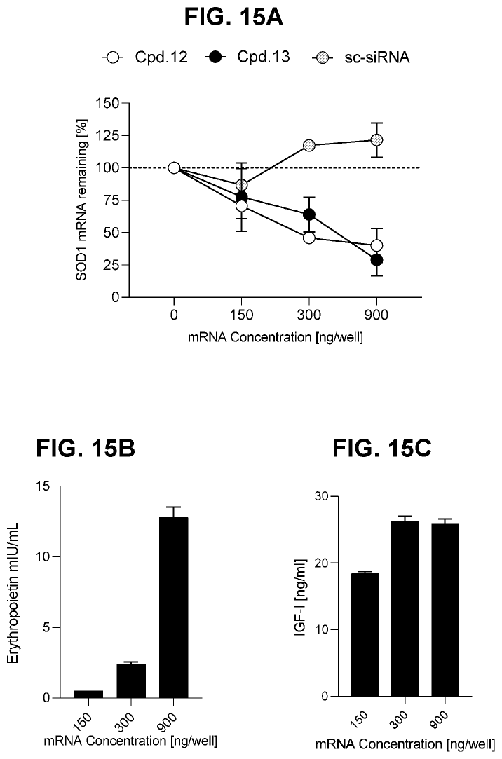

The effect on SOD1 downregulation in IMR32 cells of an escalating series of three doses of Compound A12 (comprising 3×SOD1-targeting siRNA and IGF-1 protein coding sequence) and Compound A13 (comprising 3×SOD1-targeting siRNA and EPO protein coding sequence) was evaluated (150, 300 and 900 ng/well). The assay showed that Compound A12 and Compound A13 reduced the SOD1 transcripts in a dose-dependent manner (up to at least approximately 70%) (FIG. 15A, open circles and closed circles, respectively). The scrambled siRNA did not show an effect (FIG. 15A, shaded circles). In the same cell culture supernatant (IMR32 cells), the expression of EPO protein of Compound A13 was assessed. As demonstrated in FIG. 15B, Compound A13 induced EPO expression in a dose-dependent manner. Likewise, the expression of IGF-1 protein from Compound A12 in the same IMR32 cell culture supernatant was assessed. As shown in FIG. 15C, Compound A12 simultaneously expressed IGF-1.

### Example 21: IL-1 Beta Overexpression Model in HEK-293 Cells

Compound A14 and Compound A15 were assayed for their ability to downregulate IL-1 beta expression, and overexpress IGF-1 in HEK-293 cells. An IL-1 beta overexpression model was established in HEK-293 cells using IL-1 beta mRNA transfection (300 ng/well). Compound A14 comprises siRNA targeting IL-1 beta 5′ to the IGF-1 coding sequence (upstream of the IGF-1 gene) while Compound A15 comprises siRNA targeting IL-1 beta 3′ to the IGF-1 coding sequence (downstream of the IGF-1 gene). To assess the capability of Compound A14 and Compound A15 containing siRNAs targeting IL-1 beta in IL-1 beta downregulation and simultaneous IGF-1 expression, the HEK-293 cells were co-transfected with Compound A14 or Compound A15 (900 ng/well) and IL-1 beta mRNA (300 ng/well). Post transfection, the cells were incubated at 37° C. in a humidified atmosphere containing 5% CO₂ for 24 hours followed by quantification of IL-1 beta (target gene to downregulate) and IGF-1 (Gene of Interest to overexpress) by ELISA in the same cell culture supernatant.

Results

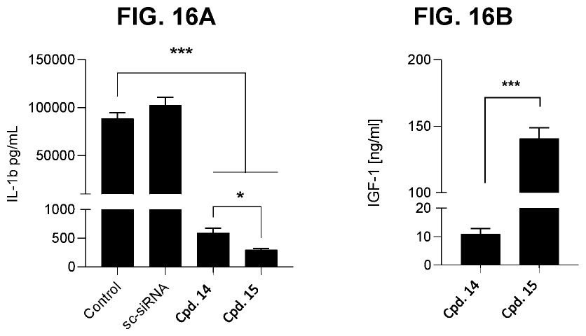

Compound A14 and Compound A15 comprise IL-1 beta-targeting siRNA either 5′ or 3′ of IGF-1 coding sequence, respectively. The constructs were tested for IL-1 beta downregulation and IGF-1 expression at the same time in HEK-293 cells (900 ng/well) with exogenously delivered IL-1 beta mRNA (300 ng/well). The data demonstrate that Compound A15 expresses approximately 13-fold higher IGF-1 than Compound A14 as shown in FIG. 16B (P<0.001). In the same experiment with the same cell culture supernatant, the RNA interference of Compound A14 and Compound A15 (900 ng/well) against IL-1 beta expression from the IL-1 beta overexpression construct (300 ng/well) was assessed, as measured by IL-1 beta ELISA. Compound A14 and Compound A15 downregulated the IL-1 beta levels by more than approximately 150-fold and 290-fold, respectively, compared to untreated control (P<0.001) as shown in FIG. 16A. Compound A15 induced at least approximately 2-fold IL-1 beta downregulation as compared to Compound A14 in which the siRNA is positioned upstream of (5′ to) the IGF-1 ORF (FIG. 16A; P<0.05). These data demonstrated that Compound A15 (having IL-1 beta-targeting siRNA positioned 3′ to the IGF-1 gene) downregulated IL-1 beta by 290-fold, and increased IGF-1 expression while significantly increasing IGF-1 expression. Compound A14 (having IL-1 beta-targeting siRNA positioned 5′ to IGF-1 gene) downregulated IL-1 beta by 150-fold, and increased IGF-1 expression while significantly increasing IGF-1 expression. Thus, Compound A15 downregulation of IL-1 beta was 2-fold greater than that observed for Compound A14. Additionally, Compound A15 expression of IGF-1 was 13 fold greater than that observed for Compound A14.

### Example 22: SARS CoV-2 Nucleocapsid Protein Overexpression Model in A549 Cells

In Vitro Transfection of A549 Cells with SARS CoV-2 Nucleocapsid Protein with eGFP Tag pCDNA3⁺ Vector and SARS CoV-2 Nucleocapsid Protein Suppressing/Soluble ACE2 Overexpression Compounds

A SARS CoV-2 Nucleocapsid protein overexpression model was used to evaluate simultaneous SARS CoV-2 Nucleocapsid (N) protein RNAi suppression and soluble ACE2 overexpression by Compound B18 in A549 cells. The model was established by transfection of a plasmid pcDNA3⁺ vector (300 ng/well) containing a SARS CoV-2 N protein with eGFP tag. The RNAi of Compound B18 targeting SARS CoV-2 N protein disrupts the downstream eGFP translation and expression. Compound B18 contains a soluble ACE2 encoding ORF and 3×SARS CoV-2-targeting siRNA (lx target ORF1ab region, lx target Spike protein and 1× target nucleocapsid protein) 3′ to (downstream of) the ACE2 ORF. The cells were co-transfected with Compound B18 (600 ng/well) and a SARS CoV-2 Nucleocapsid protein overexpressing plasmid construct (300 ng/well). Post transfection, the cells were incubated at 37° C. in a humidified atmosphere containing 5% CO₂ for 24 hours, followed by determination of whether RNAi suppression by Compound B18 leads to the disruption of eGFP translation. The SARS CoV-2 Nucleocapsid proteins tagged with eGFP (from expression of plasmid) were microscopically examined for eGFP expression using SpectraMax i3X multi-mode microplate reader (Molecular Devices). The percentage of eGFP positive cells was calculated in treated and control untreated samples.

Results

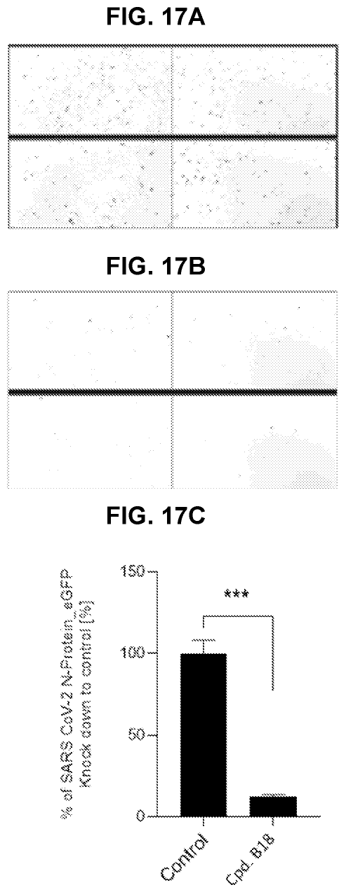

The effect of Compound B18 (comprising 3×SARS CoV-2 targeting siRNA 3′ to a soluble ACE2 protein coding sequence) was evaluated for SARS CoV-2 N-Protein downregulation in A549 cells. A reduced number of eGFP positive cells was observed, showing the targeting effect of Compound B18 against SARS CoV-2 N-Protein encoding mRNA (FIGS. 17A and 17B). The cumulative analysis from different samples showed an approximately 8-fold reduction in eGFP positive cells by Compound B18 compared to untreated control (FIG. 17C).

The examples and embodiments described herein are for illustrative purposes only and various modifications or changes suggested to persons skilled in the art are to be included within the spirit and purview of this application and scope of the appended claims.

TABLE 7
Table of Sequences Listed

| Protein or | | SEQ ID |
|---|---|---|
| Nucleic Acid | Sequence (protein: N-term to C-term; nucleic acid: 5′ to 3) | NO: |
| Compounds A1- | See Table 2 | 1-8 |
| A8 | | |
| Compounds A1- | See Table 3 | 9-16 |
| A8 (plasmid | | |
| sequences) | | |
| Forward primer | GCTGCAAGGCGATTAAGTTG | 17 |
| for template | | |
| generation | | |
| Reverse primer | U(2′OMe)U(2′OMe)U(2′OMe)TTTTTTTTTTTTTTTTTTTTTTTTTT | 18 |
| for template | TTTTTTTTTTTTTTTTTTTTTTTTTTTTTTTTTTTTTTTTTTTTTTTTTT | |
| generation | TTTTTTTTTTTTTTTTTTTTTTTTTTTTTTTTTTTTTTTTTCAGCTATGA | |
| | CCATGTTAATGCAG | |
| A mature human | GGACCTGAGACACTTTGTGGCGCTGAACTGGTGGACGCCCTGCAGTTTGT | 19 |
| IGF-1 coding | GTGTGGCGACAGAGGCTTCTACTTCAACAAGCCCACAGGCTACGGCAGCA | |
| sequence | GCTCTAGAAGGGCTCCTCAGACCGGAATCGTGGACGAGTGCTGTTTCAGA | |
| | AGCTGCGACCTGCGGCGGCTGGAAATGTATTGTGCCCCTCTGAAGCCTGC | |
| | CAAGAGCGCC | |
| A modified | MLILLLPLLLFKCFCDFLK | 20 |
| signal peptide of | | |
| IGF-1 | | |
| A modified | ATGCTGATTCTGCTGCTGCCCCTGCTGCTGTTCAAGTGCTTCTGCGACTT | 21 |
| signal peptide of | CCTGAAA | |
| IGF-1-coding | | |
| sequence | | |
| A modified | MLFYLALCLLTFTSSATA | 22 |
| IGF-1 pro | | |
| domain | | |
| A modified | ATGCTGTTCTATCTGGCCCTGTGCCTGCTGACCTTTACCAGCTCTGCTAC | 23 |
| IGF-1 pro | CGCC | |
| domain-coding | | |
| sequence | | |
| tRNA linker | AACAAAGCACCAGTGGTCTAGTGGTAGAATAGTACCCTGCCACGGTACAG | 24 |
| | ACCCGGGTTCGATTCCCGGCTGGTGCA | |
| T7 promoter | TAATACGACTCACTATA | 25 |
| Kozak sequence | GCCACC | 26 |
| Flexible linker | GGGGS | 27 |
| amino acid | | |
| Flexible linker | GGGGGTGGAGGCTCT | 28 |
| nucleic acid | | |
| Compounds B1- | See Table 5 and 6 | 29-47 |
| B19 anti-viral | | |
| nucleic acid | | |
| sequences | | |
| Human IFN- | MTNKCLLQIALLLCFSTTALSMSYNLLGFLQRSSNFQCQKLLWQLNGRLE | 48 |
| beta amino acid | YCLKDRMNFDIPEEIKQLQQFQKEDAALTIYEMLQNIFAIFRQDSSSTGW | |
| (Genbank | NETIVENLLANVYHQINHLKTVLEEKLEKEDFTRGKLMSSLHLKRYYGRI | |
| NM_002176.3) | LHYLKAKEYSHCAWTIVRVEILRNFYFINRLTGYLRN | |
| Underlined: | | |
| signal sequence | | |
| Human IFN- | CCATACCCATGGAGAAAGGACATTCTAACTGCAACCTTTCGAAGCCTTTG | 49 |
| beta nucleic acid | CTCTGGCACAACAGGTAGTAGGCGACACTGTTCGTGTTGTCAACATGACC | |
| (Genbank | AACAAGTGTCTCCTCCAAATTGCTCTCCTGTTGTGCTTCTCCACTACAGC | |
| NM_002176.3) | TCTTTCCATGAGCTACAACTTGCTTGGATTCCTACAAAGAAGCAGCAATT | |
| | TTCAGTGTCAGAAGCTCCTGTGGCAATTGAATGGGAGGCTTGAATACTGC | |
| | CTCAAGGACAGGATGAACTTTGACATCCCTGAGGAGATTAAGCAGCTGCA | |
| | GCAGTTCCAGAAGGAGGACGCCGCATTGACCATCTATGAGATGCTCCAGA | |
| | ACATCTTTGCTATTTTCAGACAAGATTCATCTAGCACTGGCTGGAATGAG | |
| | ACTATTGTTGAGAACCTCCTGGCTAATGTCTATCATCAGATAAACCATCT | |
| | GAAGACAGTCCTGGAAGAAAAACTGGAGAAAGAAGATTTCACCAGGGGAA | |
| | AACTCATGAGCAGTCTGCACCTGAAAAGATATTATGGGAGGATTCTGCAT | |
| | TACCTGAAGGCCAAGGAGTACAGTCACTGTGCCTGGACCATAGTCAGAGT | |
| | GGAAATCCTAAGGAACTTTTACTTCATTAACAGACTTACAGGTTACCTCC | |
| | GAAACTGAAGATCTCCTAGCCTGTGCCTCTGGGACTGGACAATTGCTTCA | |
| | AGCATTCTTCAACCAGCAGATGCTGTTTAAGTGACTGATGGCTAATGTAC | |
| | TGCATATGAAAGGACACTAGAAGATTTTGAAATTTTTATTAAATTATGAG | |
| | TTATTTTTATTTATTTAAATTTTATTTTGGAAAATAAATTATTTTTGGTG | |
| | CAAAAGTCA | |
| Optimized | ATGACCAACAAGTGCCTGCTGCAGATTGCCCTGCTGCTGTGCTTCAGCAC | 50 |
| Human IFN- | AACAGCCCTGAGCATGAGCTACAACCTGCTGGGCTTCCTGCAGCGGAGCA | |
| beta nucleic acid | GCAACTTCCAGTGCCAGAAACTGCTGTGGCAGCTGAACGGCCGGCTGGAA | |
| sequence | TACTGCCTGAAGGACCGGATGAACTTCGACATCCCCGAGGAAATCAAGCA | |
| encoding SEQ | GCTGCAGCAGTTCCAGAAAGAGGACGCCGCTCTGACCATCTACGAGATGC | |
| ID NO: 48 | TGCAGAACATCTTCGCCATCTTCCGGCAGGACAGCAGCTCCACAGGCTGG | |
| Underlined: | AACGAGACAATCGTGGAAAATCTGCTGGCCAACGTGTACCACCAGATCAA | |
| signal sequence | CCACCTGAAAACCGTGCTGGAAGAGAAGCTGGAAAAAGAGGACTTCACCC | |
| | GGGGCAAGCTGATGAGCAGCCTGCACCTGAAGCGGTACTACGGCAGAATC | |
| | CTGCACTACCTGAAGGCCAAAGAGTACAGCCACTGCGCCTGGACCATCGT | |
| | GCGCGTGGAAATCCTGCGGAACTTCTACTTCATCAACCGGCTGACCGGCT | |
| | ACCTGAGAAACTGA | |
| IFN-beta signal | MTNKCLLQIALLLCFSTTALS | 51 |
| peptide | | |
| (Genbank | | |
| NM_002176.3) | | |
| Modified IFN- | MLLICLLVIALLLCFSTTALS | 52 |
| beta signal | | |
| peptide (SP1) | | |
| amino acid | | |
| (T2L/N3L/K4I | | |
| and Q8V) | | |
| Modified IFN- | ATGCTCCTGATCTGCCTGCTGGTGATTGCCCTGCTGCTGTGCTTCAGCAC | 53 |
| beta signal | AACAGCCCTGAGC | |
| peptide (SP1) | | |
| nucleic acid | | |
| Modified IFN- | MLLKLLLVIALLACFSTTALS | 54 |
| beta signal | | |
| peptide (SP2) | | |
| amino acid | | |
| (T2L/N3L/C5L/ | | |
| Q8V and L13A) | | |
| Modified IFN- | ATGCTCCTGAAGCTCCTGCTGGTGATTGCCCTGCTGGCCTGCTTCAGCAC | 55 |
| beta signal | AACAGCCCTGAGC | |
| peptide (SP2) | | |
| nucleic acid | | |
| ACE2 amino | MSSSSWLLLSLVAVTAAQSTIEEQAKTFLDKFNHEAEDLFYQSSLASWNY | 56 |
| acid (Genbank | NTNITEENVQNMNNAGDKWSAFLKEQSTLAQMYPLQEIQNLTVKLQLQAL | |
| NM_021804.2) | QQNGSSVLSEDKSKRLNTILNTMSTIYSTGKVCNPDNPQECLLLEPGLNE | |
| Bold: ACE2 | IMANSLDYNERLWAWESWRSEVGKQLRPLYEEYVVLKNEMARANHYEDYG | |
| transmembrane | DYWRGDYEVNGVDGYDYSRGQLIEDVEHTFEEIKPLYEHLHAYVRAKLMN | |
| domain and | AYPSYISPIGCLPAHLLGDMWGRFWTNLYSLTVPFGQKPNIDVTDAMVDQ | |
| intracellular | AWDAQRIFKEAEKFFVSVGLPNMTQGFWENSMLTDPGNVQKAVCHPTAWD | |
| domain | LGKGDFRILMCTKVTMDDFLTAHHEMGHIQYDMAYAAQPFLLRNGANEGF | |
| (residues 741- | HEAVGEIMSLSAATPKHLKSIGLLSPDFQEDNETEINFLLKQALTIVGTL | |
| 805) | PFTYMLEKWRWMVFKGEIPKDQWMKKWWEMKREIVGVVEPVPHDETYCDP | |
| | ASLFHVSNDYSFIRYYTRTLYQFQFQEALCQAAKHEGPLHKCDISNSTEA | |
| | GQKLFNMLRLGKSEPWTLALENVVGAKNMNVRPLLNYFEPLFTWLKDQNK | |
| | NSFVGWSTDWSPYADQSIKVRISLKSALGDKAYEWNDNEMYLFRSSVAYA | |
| | MRQYFLKVKNQMILFGEEDVRVANLKPRISFNFFVTAPKNVSDIIPRTEV | |
| | EKAIRMSRSRINDAFRLNDNSLEFLGIQPTLGPPNQPPVSIWLIVFGVVM | |
| | GVIVVGIVILIFTGIRDRKKKNKARSGENPYASIDISKGENNPGFQNTDD | |
| | VQTSF | |
| ACE2 nucleic | ATGTCAAGCTCTTCCTGGCTCCTTCTCAGCCTTGTTGCTGTAACTGCTGC | 57 |
| acid encoding | TCAGTCCACCATTGAGGAACAGGCCAAGACATTTTTGGACAAGTTTAACC | |
| SEQ ID NO: 56 | ACGAAGCCGAAGACCTGTTCTATCAAAGTTCACTTGCTTCTTGGAATTAT | |
| (from Genbank | AACACCAATATTACTGAAGAGAATGTCCAAAACATGAATAATGCTGGGGA | |
| NM_021804.2) | CAAATGGTCTGCCTTTTTAAAGGAACAGTCCACACTTGCCCAAATGTATC | |
| Bold and | CACTACAAGAAATTCAGAATCTCACAGTCAAGCTTCAGCTGCAGGCTCTT | |
| italicized: | CAGCAAAATGGGTCTTCAGTGCTCTCAGAAGACAAGAGCAAACGGTTGAA | |
| siRNA binding | CACAATTCTAAATACAATGAGCACCATCTACAGTACTGGAAAAGTTTGTA | |
| regions | ACCCAGATAATCCACAAGAATGCTTATTACTTGAACCAGGTTTGAATGAA | |
| Bold: ACE2 | ATAATGGCAAACAGTTTAGACTACAATGAGAGGCTCTGGGCTTGGGAAAG | |
| transmembrane | CTGGAGATCTGAGGTCGGCAA [symbol] AGAGTATG | |
| domain and | TGGTCTTGAAAAATGAGATGGCAAGAGCAAATCATTATGAGGACTATGGG | |
| intracellular | GATTATTGGAGAGGAGACTATGAAGTAAATGGGGTAGATGGCTATGACTA | |
| domain coding | CAGCCGCGGCCAGTTGATTGAAGATGTGGAACATACCTTTGAAGAGATTA | |
| sequence | AACCATTATATGAACATCTTCATGCCTATGTGAGGGCAAAGTTGATGAAT | |
| | GCCTATCCTTCCTATATCAGTCCAATTGGATGCCTCCCTGCTCATTTGCT | |
| | TGGTGATATGTGGGGTAGATTTTGGACAAATCTGTACTCTTTGACAGTTC | |
| | CCTTTGGACAGAAACCAAACATAGATGTTACTGATGCAATGGTGGACCAG | |
| | GCCTGGGATGCACAGAGAATATTCAAGGAGGCCGAGAAGTTCTTTGTATC | |
| | TGTTGGTCTTCCTAATATGACTCAAGGATTCTGGGAAAATTCCATGCTAA | |
| | C [symbol] AGCAGTCTGCCATCCCACAGCTTGGGAC | |
| | CTGGGGAAGGGCGACTTCAGGATCCTTATGTGCACAAAGGTGACAATGGA | |
| | CGACTTCCTGACAGCTCATCATGAGATGGGGCATATCCAGTATGATATGG | |
| | CATATGCTGCACAACCTTTTCTGCTAAGAAATGGAGCTAATGAAGGATTC | |
| | CATGAAGCTGTTGGGGAAATCATGTCACTTTCTGCAGCCACACCTAAGCA | |
| | TTTAAAATCCATTGGTCTTCTGTCACCCGATTTTCAAGAAGACAATGAAA | |
| | CAGAAATAAACTTCCTGCTCAAACAAGCACTCACGATTGTTGGGACTCTG | |
| | CCATTTACTTACATGTTAGAGAAGTGGAGGTGGATGGTCTTTAAAGGGGA | |
| | AATTCCCAAAGACCAGTGGATGAAAAAGTGGTGGGAGATGAAGCGAGAGA | |
| | TAGTTGGGGTGGTGGAACCTGTGCCCCATGATGAAACATACTGTGACCCC | |
| | GCATCTCTGTTCCATGTTTCTAATGATTACTCATTCATTCGATATTACAC | |
| | AAGGACCCTTTACCAATTCCAGTTTCAAGAAGCACTTTGTCAAGCAGCTA | |
| | AACATGAAGGCCCTCTGCACAAATGTGACATCTCAAACTCTACAGAAGCT | |
| | GGACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCAC | |
| | TGCTCAACTACTTTGAGCCCTTATTTACCT [symbol] | |
| | [symbol] ATTCTTTTGTGGGATGGAGTACCGACTGGAGTCCATATGCAGACCAAAG | |
| | CATCAAAGTGAGGATAAGCCTAAAATCAGCTCTTGGAGATAAAGCATATG | |
| | AATGGAACGACAATGAAATGTACCTGTTCCGATCATCTGTTGCATATGCT | |
| | ATGAGGCAGTACTTTTTAAAAGTAAAAAATCAGATGATTCTTTTTGGGGA | |
| | GGAGGATGTGCGAGTGGCTAATTTGAAACCAAGAATCTCCTTTAATTTCT | |
| | TTGTCACTGCACCTAAAAATGTGTCTGATATCATTCCTAGAACTGAAGTT | |
| | GAAAAGGCCATCAGGATGTCCCGGAGCCGTATCAATGATGCTTTCCGTCT | |
| | GAATGACAACAGCCTAGAGTTTCTGGGGATACAGCCAACACTTGGACCTC | |
| | CTAACCAGCCCCCTGTTTCCATATGGCTGATTGTTTTTGGAGTTGTGATG | |
| | GGAGTGATAGTGGTTGGCATTGTCATCCTGATCTTCACTGGGATCAGAGA | |
| | TCGGAAGAAGAAAAATAAAGCAAGAAGTGGAGAAAATCCTTATGCCTCCA | |
| | TCGATATTAGCAAAGGAGAAAATAATCCAGGATTCCAAAACACTGATGAT | |
| | GTTCAGACCTCCTTTTAG | |
| ACE2 Soluble | MSSSSWLLLSLVAVTAAQSTIEEQAKTFLDKFNHEAEDLFYQSSLASWNY | 58 |
| Receptor- | NTNITEENVQNMNNAGDKWSAFLKEQSTLAQMYPLQEIQNLTVKLQLQAL | |
| Ectodomain | QQNGSSVLSEDKSKRLNTILNTMSTIYSTGKVCNPDNPQECLLLEPGLNE | |
| amino acid | IMANSLDYNERLWAWESWRSEVGKQLRPLYEEYVVLKNEMARANHYEDYG | |
| sequence | DYWRGDYEVNGVDGYDYSRGQLIEDVEHTFEEIKPLYEHLHAYVRAKLMN | |
| (derived from | AYPSYISPIGCLPAHLLGDMWGRFWTNLYSLTVPFGQKPNIDVTDAMVDQ | |
| Genbank | AWDAQRIFKEAEKFFVSVGLPNMTQGFWENSMLTDPGNVQKAVCHPTAWD | |
| NM_021804.2; | LGKGDFRILMCTKVTMDDFLTAHHEMGHIQYDMAYAAQPFLLRNGANEGF | |
| does not include | HEAVGEIMSLSAATPKHLKSIGLLSPDFQEDNETEINFLLKQALTIVGTL | |
| transmembrane | PFTYMLEKWRWMVFKGEIPKDQWMKKWWEMKREIVGVVEPVPHDETYCDP | |
| domain and | ASLFHVSNDYSFIRYYTRTLYQFQFQEALCQAAKHEGPLHKCDISNSTEA | |
| intracellular | GQKLFNMLRLGKSEPWTLALENVVGAKNMNVRPLLNYFEPLFTWLKDQNK | |
| domain) | NSFVGWSTDWSPYADQSIKVRISLKSALGDKAYEWNDNEMYLFRSSVAYA | |
| | MRQYFLKVKNQMILFGEEDVRVANLKPRISFNFFVTAPKNVSDIIPRTEV | |
| | EKAIRMSRSRINDAFRLNDNSLEFLGIQPTLGPPNQPPVS | |
| ACE2 Soluble | ATGTCAAGCTCTTCCTGGCTCCTTCTCAGCCTTGTTGCTGTAACTGCTGC | 59 |
| Receptor- | TCAGTCCACCATTGAGGAACAGGCCAAGACATTTTTGGACAAGTTTAACC | |
| Ectodomain | ACGAAGCCGAAGACCTGTTCTATCAAAGTTCACTTGCTTCTTGGAATTAT | |
| nucleic acid | AACACCAATATTACTGAAGAGAATGTCCAAAACATGAATAATGCTGGGGA | |
| sequence | CAAATGGTCTGCCTTTTTAAAGGAACAGTCCACACTTGCCCAAATGTATC | |
| encoding SEQ | CACTACAAGAAATTCAGAATCTCACAGTCAAGCTTCAGCTGCAGGCTCTT | |
| ID NO: 58 | CAGCAAAATGGGTCTTCAGTGCTCTCAGAAGACAAGAGCAAACGGTTGAA | |
| Underlined: | CACAATTCTAAATACAATGAGCACCATCTACAGTACTGGAAAAGTTTGTA | |
| signal sequence | ACCCAGATAATCCACAAGAATGCTTATTACTTGAACCAGGTTTGAATGAA | |
| (derived from | ATAATGGCAAACAGTTTAGACTACAATGAGAGGCTCTGGGCTTGGGAAAG | |
| Genbank | CTGGAGATCTGAGGTCGGCAAGCAGCTGAGGCCATTATATGAAGAGTATG | |
| NM_021804.2; | TGGTCTTGAAAAATGAGATGGCAAGAGCAAATCATTATGAGGACTATGGG | |
| does not include | GATTATTGGAGAGGAGACTATGAAGTAAATGGGGTAGATGGCTATGACTA | |
| transmembrane | CAGCCGCGGCCAGTTGATTGAAGATGTGGAACATACCTTTGAAGAGATTA | |
| domain and | AACCATTATATGAACATCTTCATGCCTATGTGAGGGCAAAGTTGATGAAT | |
| intracellular | GCCTATCCTTCCTATATCAGTCCAATTGGATGCCTCCCTGCTCATTTGCT | |
| domain coding | TGGTGATATGTGGGGTAGATTTTGGACAAATCTGTACTCTTTGACAGTTC | |
| sequence) | CCTTTGGACAGAAACCAAACATAGATGTTACTGATGCAATGGTGGACCAG | |
| | GCCTGGGATGCACAGAGAATATTCAAGGAGGCCGAGAAGTTCTTTGTATC | |
| | TGTTGGTCTTCCTAATATGACTCAAGGATTCTGGGAAAATTCCATGCTAA | |
| | CGGACCCAGGAAATGTTCAGAAAGCAGTCTGCCATCCCACAGCTTGGGAC | |
| | CTGGGGAAGGGCGACTTCAGGATCCTTATGTGCACAAAGGTGACAATGGA | |
| | CGACTTCCTGACAGCTCATCATGAGATGGGGCATATCCAGTATGATATGG | |
| | CATATGCTGCACAACCTTTTCTGCTAAGAAATGGAGCTAATGAAGGATTC | |
| | CATGAAGCTGTTGGGGAAATCATGTCACTTTCTGCAGCCACACCTAAGCA | |
| | TTTAAAATCCATTGGTCTTCTGTCACCCGATTTTCAAGAAGACAATGAAA | |
| | CAGAAATAAACTTCCTGCTCAAACAAGCACTCACGATTGTTGGGACTCTG | |
| | CCATTTACTTACATGTTAGAGAAGTGGAGGTGGATGGTCTTTAAAGGGGA | |
| | AATTCCCAAAGACCAGTGGATGAAAAAGTGGTGGGAGATGAAGCGAGAGA | |
| | TAGTTGGGGTGGTGGAACCTGTGCCCCATGATGAAACATACTGTGACCCC | |
| | GCATCTCTGTTCCATGTTTCTAATGATTACTCATTCATTCGATATTACAC | |
| | AAGGACCCTTTACCAATTCCAGTTTCAAGAAGCACTTTGTCAAGCAGCTA | |
| | AACATGAAGGCCCTCTGCACAAATGTGACATCTCAAACTCTACAGAAGCT | |
| | GGACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCAC | |
| | TGCTCAACTACTTTGAGCCCTTATTTACCTGGCTGAAAGACCAGAACAAG | |
| | AATTCTTTTGTGGGATGGAGTACCGACTGGAGTCCATATGCAGACCAAAG | |
| | CATCAAAGTGAGGATAAGCCTAAAATCAGCTCTTGGAGATAAAGCATATG | |
| | AATGGAACGACAATGAAATGTACCTGTTCCGATCATCTGTTGCATATGCT | |
| | ATGAGGCAGTACTTTTTAAAAGTAAAAAATCAGATGATTCTTTTTGGGGA | |
| | GGAGGATGTGCGAGTGGCTAATTTGAAACCAAGAATCTCCTTTAATTTCT | |
| | TTGTCACTGCACCTAAAAATGTGTCTGATATCATTCCTAGAACTGAAGTT | |
| | GAAAAGGCCATCAGGATGTCCCGGAGCCGTATCAATGATGCTTTCCGTCT | |
| | GAATGACAACAGCCTAGAGTTTCTGGGGATACAGCCAACACTTGGACCTC | |
| | CTAACCAGCCCCCTGTTTCCTAA | |
| SARS CoV-2 | RVQPTESIVRFPNITNLCPFGEVFNATRFASVYAWNRKRISNCVADYSVL | 60 |
| Spike RBD | YNSASFSTFKCYGVSPTKLNDLCFTNVYADSFVIRGDEVRQIAPGQTGKI | |
| amino acid | ADYNYKLPDDFTGCVIAWNSNNLDSKVGGNYNYLYRLFRKSNLKPFERDI | |
| sequence | STEIYQAGSTPCNGVEGFNCYFPLQSYGFQPTNGVGYQPYRVVVLSFELL | |
| | HAPATVCGPKKSTNLVKNKCVNF | |
| SARS CoV-2 | AGAGTCCAACCAACAGAATCTATTGTTAGATTTCCTAATATTACAAACTT | 61 |
| Spike RBD | GTGCCCTTTTGGTGAAGTTTTTAACGCCACCAGATTTGCATCTGTTTATG | |
| nucleic acid | CTTGGAACAGGAAGAGAATCAGCAACTGT [symbol] | |
| sequence | TATAATTCCGCATCATTTTCCACTTTTAAGTGTTATGGAGTGTCTCCTAC | |
| (encoding SEQ | TAAATTAAATGATCTCTGCTTTACTAATGTCTATGCAGATTCATTTGTAA | |
| ID NO: 36) | TTA [symbol] TCGCTCCAGGGCAAACTGGAAAGATT | |
| Bold and | GCTGATTATAATTATAAATTACCAGATGATTTTACAGGCTGCGTTATAGC | |
| italicized: | TTGGAATTCTAACAATCTTGATTCTAAGGTTGGTGGTAATTATAATTACC | |
| siRNA binding | TGTATAGATTGTTTAGGAAGTCTAATCTCAAACCTTTTGAGAGAGATATT | |
| regions | TCAACTGAAATCTATCAG [symbol] GGTGTTGAAGG | |
| | TTTTAATTGTTACTTTCCTTTACAATCATATGGTTTCCAACCCACTAATG | |
| | GTGTTGGTTACCAACCATACAGAGTAGTAGTACTTTCTTTTGAACTTCTA | |
| | CATGCACCAGCAACTGTTTGTGGACCTAAAAAGTCTACTAATTTGGTTAA | |
| | AAACAAATGTGTCAATTTC | |
| SARS CoV-2 | MSDNGPQNQRNAPRITFGGPSDSTGSNQNGERSGARSKQRRPQGLPNNTA | 62 |
| Nucleocapsid | SWFTALTQHGKEDLKFPRGQGVPINTNSSPDDQIGYYRRATRRIRGGDGK | |
| protein (N) | MKDLSPRWYFYYLGTGPEAGLPYGANKDGIIWVATEGALNTPKDHIGTRN | |
| amino acid | PANNAAIVLQLPQGTTLPKGFYAEGSRGGSQASSRSSSRSRNSSRNSTPG | |
| sequence (NCBI | SSRGTSPARMAGNGGDAALALLLLDRLNQLESKMSGKGQQQQGQTVTKKS | |
| YP_009724397.2) | AAEASKKPRQKRTATKAYNVTQAFGRRGPEQTQGNFGDQELIRQGTDYKH | |
| | WPQIAQFAPSASAFFGMSRIGMEVTPSGTWLTYTGAIKLDDKDPNFKDQV | |
| | ILLNKHIDAYKTFPPTEPKKDKKKKADETQALPQRQKKQQTVTLLPAADL | |
| | DDFSKQLQQSMSSADSTQA | |
| SARS CoV-2 | GCCACCATGTCTGATAATGGACCCCAAAATCAGCGAAATGCACCCCGCAT | 63 |
| Nucleocapsid | TACGTTTGGTGGACCCTCAGATTCAACTGGCAGTAACCAGAATGGAGAAC | |
| protein (N) | GCAGTGGGGCGCGATCAAAACAACGTCGGCCCCAAGGTTTACCCAATAAT | |
| nucleic acid | ACTGCGTCTTGGTTCACCGCTCTCACTCAACATGGCAAGGAAGACCTTAA | |
| sequence | ATTCCCTCGAGGACAAGGCGTTCCAATTAACACCAATAGCAGTCCAGATG | |
| encoding SEQ | ACCAAATTGGCTACTACCGAAGAGCTACCAGACGAATTCGTGGTGGTGAC | |
| ID NO: 38 | GGTAAAATGAAAGATCTCAGTCCAAGATGGTATTTCTACTACCTAGGAAC | |
| Bold and | TGGGCCAGAAGCTGGACTTCCCTATGGTGCTAACAAAGACGGCATCATAT | |
| underlined: | GGGTT [symbol] ACACCAAAAGATCACATTGGCACC | |
| Kozak sequence | CGCAATCCTGCTAACAATGCTGCAATCGTGCTACAACTTCCTCAAGGAAC | |
| Italicized: ORF | AACATTGCCAAAAGGCTTCTACGCAGAAGGGAGCAGAGGCGGCAGTCAAG | |
| of SARS CoV-2 | CCTCTTCTCGTTCCTCATCACGTAGTCGCAACAGTTCAAGAAATTCAACT | |
| Nucleocapsid | CCAGGCAGCAGTAGGGGAACTTCTCCTGCTAGAATGGCTGGCAATGGCGG | |
| (N) protein | TGATGCTGCTCTTGCTTTGCTGCTGCTTGACAGATTGAACCAGCTTGAGA | |
| Bold and | GCAAAATGTCTGGTAAAGGCCAACAACAACAAGGCCAAACTGTCACTAAG | |
| italicized: | AAATCTGCTGCTGAGGCTTCTAAGAAGCCTCGGCAAAAACGTACTGCCAC | |
| siRNA binding | TAAAGCATACAATGTAACACAAGCTTTCGGCAGACGTGGTCCAGAACAAA | |
| region | CCCAAGGAAATTTTGGGGACCAGGAACTAATCAGACAAGGAACTGATTAC | |
| Bold: Flexible | AAACATTGGCCGCAAATTGCACAATTTGCCCCCAGCGCTTCAGCGTTCTT | |
| Linker | CGGAATGTCGCGCATTGGCATGGAAGTCACACCTTCGGGAACGTGGTTGA | |
| Underlined: | CCTACACAGGTGCCATCAAATTGGATGACAAAGATCCAAATTTCAAAGAT | |
| ORF of eGFP | CAAGTCATTTTGCTGAATAAGCATATTGACGCATACAAAACATTCCCACC | |
| reporter protein | AACAGAGCCTAAAAAGGACAAAAAGAAGAAGGCTGATGAAACTCAAGCCT | |
| | TACCGCAGAGACAGAAGAAACAGCAAACTGTGACTCTTCTTCCTGCTGCA | |
| | GATTTGGATGATTTCTCCAAACAATTGCAACAATCCATGAGCAGTGCTGA | |
| | CTCAACTCAGGCCGGGGGTGGAGGCTCTGTGTCCAAGGGCGAAGAACTGT | |
| | TCACCGGCGTGGTGCCCATTCTGGTGGAACTGACGGGGATGTGAACGGC | |
| | CACAAGTTTAGCGTTAGCGGCGAAGGCGAAGGGGATGCCACATACGGAAA | |
| | GCTGACCCTGAAGTTCATCTGCACCACCGGCAAGCTGCCTGTGCCTTGGC | |
| | CTACACTGGTCACCACACTGACATACGGCGTGCAGTGCTTCAGCAGATAC | |
| | CCCGACCATATGAAGCAGCACGACTTCTTCAAGAGCGCCATGCCTGAGGG | |
| | CTACGTGCAAGAGCGGACCATCTTCTTTAAGGACGACGGCAACTACAAGA | |
| | CCAGGGCCGAAGTGAAGTTCGAGGGCGACACCCTGGTCAACCGGATCGAG | |
| | CTGAAGGGCATCGACTTCAAAGAGGACGGCAACATCCTGGGCCACAAGCT | |
| | CGAGTACAACTACAACAGCCACAACGTGTACATCATGGCCGACAAGCAGA | |
| | AAAACGGCATCAAAGTGAACTTCAAGATCCGGCACAACATCGAGGACGGC | |
| | TCTGTGCAGCTGGCCGATCACTACCAGCAGAACACACCCATCGGAGATGG | |
| | CCCTGTGCTGCTGCCCGATAACCACTACCTGAGCACACAGAGCGCCCTGA | |
| | GCAAGGACCCCAACGAGAAGAGGGATCACATGGTGCTGCTGGAATTCGTG | |
| | ACCGCCGCTGGCATCACACTCGGCATGGATGAGCTGTACAAGTGA | |
| SARS CoV-2 | MESLVPGFNEKTHVQLSLPVLQVRDVLVRGFGDSVEEVLSEARQHLKDGT | 64 |
| NSP1 protein | CGLVEVEKGVLPQLEQPYVFIKRSDARTAPHGHVMVELVAELEGIQYGRS | |
| (NCBI | GETLGVLVPHVGEIPVAYRKVLLRKNGNKGAGGHSYGADLKSFDLGDELG | |
| YP_009725297.1) | TDPYEDFQENWNTKHSSGVTRELMRELNGG | |
| SARS CoV-2 | MESLVPGFNEKTHVQLSLPVLQVRDVLVRGFGDSVEEVLSEARQHLKDGT | 65 |
| NSP1 protein | CGLVEVEKGVLPQLEQPYVFIKRSDARTAPHGHVMVELVAELEGIQYGRS | |
| (first 100 amino | | |
| acids of SEQ ID | | |
| NO: 40) | | |
| SARS CoV-2 | GACACGAGTAACTCGTCTATCTTCTGCAGGCTGCTTACGGTTTCGTCCGT | 66 |
| NSP1 protein | GTTGCAGCCGATCATCAGCACATCTAGGTTTCGTCCGG [symbol] | |
| nucleic acid | [symbol] GAGAGCCTTGTCCCTGGTTTCAACGAGAAAACACACGTCCA | |
| sequence | ACTCAGTTTGCCTGTTTTACAGGTTCGCGACGTGCTCGTACGTGGCTTTG | |
| (encoding SEQ | GAGACTCCGTGGAGGAGGTCTTATCAGAGGCACGTCAACATCTTAAAGAT | |
| ID NO: 40 at | GGCACTTGTGGCTTAGTAGAAGTTGAAAAAGGCGTTTTGCCTCAACTTGA | |
| positions 107 to | ACAGCCCTATGTGTTCATCAAACGTTCGGATGCTCGAACTGCACCTCATG | |
| 406, ORF | GTCATGTTATGGTTGAGCTGGTAGCAGAACTCGAAGGCATTCAGTACGGT | |
| italicized) | CGTAGTGGGGGTGGAGGCTCTGTGTCCAAGGGCGAAGAACTGTTCACCGG | |
| Bold and | CGTGGTGCCCATTCTGGTGGAACTGGACGGGGATGTGAACGGCCACAAGT | |
| italicized: | TTAGCGTTAGCGGCGAAGGCGAAGGGGATGCCACATACGGAAAGCTGACC | |
| siRNA binding | CTGAAGTTCATCTGCACCACCGGCAAGCTGCCTGTGCCTTGGCCTACACT | |
| region | GGTCACCACACTGACATACGGCGTGCAGTGCTTCAGCAGATACCCCGACC | |
| Bold: Flexible | ATATGAAGCAGCACGACTTCTTCAAGAGCGCCATGCCTGAGGGCTACGTG | |
| Linker | CAAGAGCGGACCATCTTCTTTAAGGACGACGGCAACTACAAGACCAGGGC | |
| Underlined: | CGAAGTGAAGTTCGAGGGCGACACCCTGGTCAACCGGATCGAGCTGAAGG | |
| ORF of eGFP | GCATCGACTTCAAAGAGGACGGCAACATCCTGGGCCACAAGCTCGAGTAC | |
| reporter protein | AACTACAACAGCCACAACGTGTACATCATGGCCGACAAGCAGAAAAACGG | |
| (The 5′ UTR of | CATCAAAGTGAACTTCAAGATCCGGCACAACATCGAGGACGGCTCTGTGC | |
| SARS CoV-2 is | AGCTGGCCGATCACTACCAGCAGAACACACCCATCGGAGATGGCCCTGTG | |
| shown upstream | CTGCTGCCCGATAACCACTACCTGAGCACACAGAGCGCCCTGAGCAAGGA | |
| of the first ATG | CCCCAACGAGAAGAGGGATCACATGTGCTGCTGGAATTCGTGACCGCCG | |
| codon at | CTGGCATCACACTCGGCATGGATGAGCTGTACAAGTGA | |
| position 107) | | |
| SARS CoV-2 | GTCACATGTTGACACTGACTTAACAAAGCCTTACATTAAGTGGGATTTGT | 67 |
| NSP12 and | TAAAATATGACTTCACGGAAGAGAGGTTAAAACTCTTTGACCGTTAT [symbol] | |
| NSP13 nucleic | [symbol] ATACCACCCAAATTGTGTTAACTGTTTGGATGA | |
| acid sequence | CAGATGCATTCTGCATTGTGCAAACTTTAATGTTTTATTCTCTACAGTGT | |
| Bold and | TCCCACCTACAAGTTTTGGACCACTAGTGAGAAAAATATTTGTTGATGGT | |
| italicized: | GTTCCATTTGTAGTTTCAACTGGATACCACTTCAGAGAGCTAGGTGTTGT | |
| siRNA binding | ACATAATCAGGATGTAAACTTACATAGCTCTAGACTTAGTTTTAAGGAAT | |
| regions | TACTTGTGTATGCTGCTGACCCTGCTATGCACGCTGCTTCTGGTAATCTA | |
| | TTACTAGATAAACGCACTACGTGCTTTTCAGTAGCTGCACTTACTAACAA | |
| | TGTTGCTTTTCAAACTGTCAAACCCGGTAATTTTAACAAAGACTTCTATG | |
| | ACTTTGCTGTGTCTAAGGGTTTCTTTAAGGAAGGAAGTTCTGTTGAATTA | |
| | AAACACTTCTTCTTTGCTCAGGATGGTAATGCTGCTATCAGCGATTATGA | |
| | CTACTATCGTTATAATCTACCAACAATGTGTGATATCAGACAACTACTAT | |
| | TTGTAGTTGAAGTTGTTGATAAGTACTTTGATTGTTACGATGGTGGCTGT | |
| | ATTAATGCTAACCAAGTCATCGTCAACAACCTAGACAAATCAGCTGGTTT | |
| | TCCATTTAATAAATGGGGTAAGGCTAGACTTTATTATGATTCAATGAGTT | |
| | ATGAGGATCAAGATGCACTTTTCGCATATACAAAACGTAATGTCATCCCT | |
| | ACTATAACTCAAATGAATCTTAAGTATGCCATTAGTGC [symbol] | |
| | [symbol] CGTAGCTGGTGTCTCTATCTGTAGTACTATGACCAATAGACAGT | |
| | TTCATCAAAAATTATTGAAATCAATAGCCGCCACTAGAGGAGCTACTGTA | |
| | GTAATTGGAACAAGCAAATTCTATGGTGGTTGGCACAACATGTTAAAAAC | |
| | TGTTTATAGTGATGTAGAAAACCCTCACCTTATGGGTTGGGATTATCCTA | |
| | AATGTGATAGAGCCATGCCTAACATGCTTAGAATTATGGCCTCACTTGTT | |
| | CTTGCTCGCAAACATACAACGTGTTGTAGCTTGTCACACCGTTTCTATAG | |
| | ATTAGCTAATGAGTGTGCTCAAGTATTGAGTGAAATGGTCATGTGTGGCG | |
| | GTTCACTATATGTTAAACCAGGTGGAACCTCATCAGGAGATGCCACAACT | |
| | GCTTATGCTAATAGTGTTTTTAACATTTGTCAAGCTGTCACGGCCAATGT | |
| | TAATGCACTTTTATCTACTGATGGTAACAAAATTGCCGATAAGTATGTCC | |
| | GCAATTTACAACACAGACTTTATGAGTGTCTCTATAGAAATAGAGATGTT | |
| | GACACAGACTTTGTGAATGAGTTTTACGCATATTTGCGTAAACATTTCTC | |
| | AATGATGATACTCTCTGACGATGCTGTTGTGTGTTTCAATAGCACTTATG | |
| | CATCTCAAGGTCTAGTGGCTAGCATAAAGAACTTTAAGTCAGTTCTTTAT | |
| | TATCAAAACAATGTTTTTATGTCTGAAGCAAAATGTTGGACTGAGACTGA | |
| | CCTTACTAAAGGACCTCATGAATTTTGCTCTCAACATACAATGCTAGTTA | |
| | AACAGGGTGATGATTATGTGTACCTTCCTTACCCAGATCCATCAAGAATC | |
| | CTAGGGGCCGGCTGTTTTGTAGATGATATCGTAAAAACAGATGGTACACT | |
| | TATGATTGAACGGTTCGTGTCTTTAGCTATAGATGCTTACCCACTTACTA | |
| | AACATCCTAATCAGGAGTATGCTGATGTCTTTCATTTGTACTTACAATAC | |
| | ATAAGAAAGCTACATGATGAGTTAACAGGACACATGTTAGACATGTATTC | |
| | TGTTATGCTTACTAATGATAACACTTCAAGGTATTGGGAACCTGAGTTTT | |
| | ATGAGGCTATGTACACACCGCATACAGTCTTACAGGCTGTTGGGGCTTGT | |
| | GTTCTTTGCAATTCACAGACTTCATTAAGATGTGGTGCTTGCATACGTAG | |
| | ACCATTCTTATGTTGTAAATGCTGTTACGACCATGTCATATCAACATCAC | |
| | ATAAATTAGTCTTGTCTGTTAATCCGTATGTTTGCAATGCTCCAGGTTGT | |
| | GATGTCACAGATGTGACTCAACTTTACTTAGGAGGTATGAGCTATTATTG | |
| | TAAATCACATAAACCACCCATTAGTTTTCCATTGTGTGCTAATGGACAAG | |
| | TTTTTGGTTTATATAAAAATACATGTGTTGGTAGCGATAATGTTACTGAC | |
| | TTTAATGCAATTGCAACATGTGACTGGACAAATGCTGGTGATTACATTTT | |
| | AGCTAACACCTGTACTGAAAGACTCAAGCTTTTTGCAGCAGAAACGCTCA | |
| | AAGCTACTGAGGAGACATTTAAACTGTCTTATGGTATTGCTACTGTACGT | |
| | GAAGTGCTGTCTGACAGAGAATTACATCTTTCATGGGAAGTTGGTAAACC | |
| | TAGACCACCACTTAACCGAAATTATGTCTTTACTGGTTATCGTGTAACTA | |
| | AAAACAGTAAAGTACAAATAGGAGAGTACACCTTTGAAAAAGGTGACTAT | |
| | GGTGATGCTGTTGTTTACCGAGGTACAACAACTTACAAATTAAATGTTGG | |
| | TGATTATTTTGTGCTGACATCACATACAGTAATGCCATTAAGTGCACCTA | |
| | CACTAGTGCCACAAGAGCACTATGTTAGAATTACTGGCTTATACCCAACA | |
| | CTCAATATCTCAGATGAGTTTTCTAGCAATGTTGCAAATTATCAAAAGGT | |
| | TGGTATGCAAAAGTATTCTACACTCCAGGGACCACCTGGTACTGGTAAGA | |
| | GTCATTTTGCTATTGGCCTAGCTCTCTACTACCCTTCTGCTCGCATAGTG | |
| | TATACAGCTTGCTCTCATGCCGCTGTTGATGCACTATGTGAGAAGGCATT | |
| | AAAATATTTGCCTATAGATAAATGTAGTAGAATTATACCTGCACGTGCTC | |
| | GTGTAGAGTGTTTTGATAAATTCAAAGTGAATTCAACATTAGAACAGTAT | |
| | GTCTTTTGTACTGTAAATGCATTGCCTGAGACGACAGCAGATATAGTTGT | |
| | CTTTGATGAAATTTCAATGGCCACAAATTATGATTTGAGTGTTGTCAATG | |
| | CCAGATTACGTGCTAAGCACTATGTGTACATTGGCGACCCTGCTCAATTA | |
| | CCTGCACCACGCACATTGCTAACTAAGGGCACACTAGAACCAGAATATTT | |
| | CAATTCAGTGTGTAGACTTATGAAAACTATAGGTCCAGACATGTTCCTCG | |
| | GAACTTGTCGGCGTTGTCCTGCTGAAATTGTTGACACTGTGAGTGCTTTG | |
| | GTTTATGATAATAAGCTTAAAGCACATAAAGACAAATCAGCTCAATGCTT | |
| | TAAAATGTTTTATAAGGGTGTTATCACGCATGATGTTTCATCTGCAATTA | |
| | ACAGGCCACAAATAGGCGTGGTAAGAGAATTCCTTACACGTAACCCTGCT | |
| | TGGAGAAAAGCTGTCTTTATTTCACCTTATAATTCACAGAATGCTGTAGC | |
| | CTCAAAGATTTTGGGACTACCAACTCAA [symbol] CT | |
| | CAGAATATGACTATGTCATATTCACTCAAACCACTGAAACAGCTCACTCT | |
| | TGTAATGTAAACAGATTTAATGTTGCTATTACCAGAGCAAAAGTAGGCA | |
| qPCR Set 1 | GATGTGGTGCTTGCATACGT | 68 |
| Primer | | |
| Forward-1 | | |
| qPCR Set 1 | TGCTGTTACGACCATGTCAT | 69 |
| Probe-1 | | |
| qPCR Set 1 | TCACAACCTGGAGCATTGCA | 70 |
| Primer | | |
| Reverse-1 | | |
| qPCR Set 2 | AATAGAGCTCGCACCGTAGC | 71 |
| Primer | | |
| Forward-2 | | |
| qPCR Set 2 | GGTGTCTCTATCTGTAGTACTATGACC | 72 |
| Probe-2 | | |
| qPCR Set 2 | AGTGGCGGCTATTGATTTCA | 73 |
| Primer | | |
| Reverse-2 | | |
| IL-6 nucleic | ATGAACTCCTTCTCCACAAGCGCCTTCGGTCCAGTTGCCTTCTCCCTGGG | 74 |
| acid sequence | GCTGCTCCTGGTGTTGCCTGCTGCCTTCCCTGCCCCAGTACCCCCAGGAG | |
| (protein coding | AAGATTCCAAAGATGTAGCCGCCCCACACAGACAGCCACTCACCTCTTCA | |
| sequence) | GAACGAATTGACAAACAAATTCGGTACATCCTCGACGGCATCTCA [symbol] | |
| Bold and | [symbol] AACAAGAGTAACATGTGTGAAAGCAGCAAAGAGG | |
| italicized: | GAGTGGCAGAAAACAACCTGAACCTTCGAAAGATGGCTGAAAAAGATGGA | |
| siRNA binding | TGCTTCCAATCTGGATTCAAT [symbol] ATCATGAG | |
| regions | TGGTCTTTTGGAGTTTGAGGTATACCTAGAGTACCTCCAGAACAGATTTG | |
| | AGAGTAGTGAGGAACAAGCCAGAGCTGTGCAGATGAGTACAAAAGTCCTG | |
| | ATCCAGTTCCTGCAGAAAAAGGCAAAGAATCTAGATGCAATAACCACCCC | |
| | TGACCCAACCACAAATGCCAGCCTGCTGACGAAGCTGCAGGCACAGAACC | |
| | AGTGGCTGCAGGACATGACAACTCATCTCATTCTGCGCAGCTTTAAGGAG | |
| | TTCCTGCAGTCCAGCCT [symbol] G | |
| IL-6R-alpha | ATGCTGGCCGTCGGCTGCGCGCTGCTGGCTGCCCTGCTGGCCGCGCCGGG | 75 |
| nucleic acid | AGCGGCGCTGGCCCCAAGGCGCTGCCCTGCGCAGGAGGTGGCGAGAGGCG | |
| sequence | TGCTGACCAGTCTGCCAGGAGACAGCGTGACTCTGACCTGCCCGGGGGTA | |
| (protein coding | GAGCCGGAAGACAATGCCACTGTTCACTGGGTGCTCAGGAAGCCGGCTGC | |
| sequence) | AGGCTCCCACCCCAGCAGATGGGCTGGCATGGGAAGGAGGCTGCTGCTGA | |
| Bold and | GGTCGGTGCAGCTCCACGACTCTGGAAACTATTCATGCTACCGGGCCGGC | |
| italicized: | CGCCCAGCTGGGACTGTGCACTTGCTGGTGGATGTTCCCCCCGAGGAGCC | |
| siRNA binding | CCAGCTCTCCTGCTTCCGGAAGAGCCCCCTCAGCAATGTTGTTTGTGAGT | |
| regions | GGGGTCCTCGGAGCACCCCATCCCTGACGACAAAGGCTGTGCTCTTG [symbol] | |
| | [symbol] GCGGCCGAAGACTTCCAGGAGCCGTGCCAGTA | |
| | TTCCCAGGAGTCCCAGAAGTTCTCCTGCCAGTTAGCAGTCCCGGAGGGAG | |
| | ACAGCTCTTTCTACATAGTGTCCATGTGCGTCGCCAGTAGTGTCGGGAGC | |
| | AAGTTCAGCAAAACTCAAACCTTTCAGGGTTGTGGAATCTTGCAGCCTGA | |
| | TCCGCCTGCCAACATCACAGTCACTGCCGTGGCCAGAAACCCCCGCTGGC | |
| | TCAGTGTCACCTGGCAAGACCCCCACTCCTGGAACTCATCTTTCTACAGA | |
| | CTACGGTTTGAGCTCAGATATCGGGCT [symbol] AC | |
| | ATGGATGGTCAAGGACCTCCAGCATCACTGTGTCATCCACGACGCCTGGA | |
| | GCGGCCTGAGGCACGTGGTGCAGCTTCGTGCCCAGGAGGAGTTCGGGCAA | |
| | GGCGAGTGGAGCGAGTGGAGCCCGGAGGCCATGGGCACGCCTTGGACAGA | |
| | CAGGCTTTCTCCTCGTTGCCCAGGATGGAGTACAGCAGTGCAATCACAGC | |
| | TCACGGCAACTTCTGCCTCCTGGGTTCAAGCAATCCTCCCGCCTCAGCCT | |
| | CCTAAGTAG | |
| IL-6R-beta | ATGTTGACGTTGCAGACTTGGCTAGTGCAAGCCTTGTTTATTTTCCTCAC | 76 |
| nucleic acid | CACTGAATCTACAGGTGAACTTCTAGATCCATGTGGTTATATCAGTCCTG | |
| sequence | AATCTCCAGTTGTACAACTTCATTCTAATTTCACTGCAGTTTGTGTGCTA | |
| (protein coding | AAGGAAAAATGTATGGATTATTTTCATGTAAATGCTAATTACATTGTCTG | |
| sequence) | GAAAACAAACCATTTTACTATTCCTAAGGAGCAATATACTATCATAAACA | |
| Bold and | GAACAGCATCCAGTGTCACCTTTACAGATATAGCTTCATTAAATATTCAG | |
| italicized: | CTCACTTGCAACATTCTTACATTCGGACAGCTTGAACAGAATGTTTATGG | |
| siRNA binding | AATCACAATAATTTCAGGCTTGCCTCCAGAAAAACCTAAAAATTTGAGTT | |
| regions | GCATTGTGAACGAGGGGAAGAAAATGAGGTGTGAGTGGGATGGTGGAAGG | |
| | GAAACACACTTGGAGACAAACTTCACTTTAAAATCTGAATGGGCAACACA | |
| | CAAGTTTGCTGATTGCAAAGCAAAACGTGACACCCCCACCTCATGCACTG | |
| | TTGATTATTCTACTGTGTATTTTGTCAACATTGAAGTCTGGGTAGAAGCA | |
| | GAGAATGCCCTT [symbol] ATCAATTTTGATCCTGT | |
| | ATATAAAGTGAAGCCCAATCCGCCACATAATTTATCAGTGATCAACTCAG | |
| | AGGAACTGTCTAGTATCTTAAAATTGACATGGACCAACCCAAGTATTAAG | |
| | AGTGTTATAATACTAAAATATAACATTCAATATAGGACCAAAGATGCCTC | |
| | AACTTGGAGCCAGATTCCTCCTGAAGACACAGCATCCACCCGATCTTCAT | |
| | TCACTGTCCAAGACCTTAAACCTTTTACAGAATATGTGTTTAGGATTCGC | |
| | TGTATGAAGGAAGATGGTAAGGGATACTGGAGTGACTGGAGTGAAGAAGC | |
| | AAGTGGGATCACCTATGAAGATAACATTGCCTCCTTTTGA | |
| SARS CoV- | ATTAAAGGTTTATACCTTCCCAGGTAACAAACCAACCAACTTTCGATCTC | 77 |
| 2_Refseq | TTGTAGATCTGTTCTCTAAACGAACTTTAAAATCTGTGTGGCTGTCACTC | |
| | GGCTGCATGCTTAGTGCACTCACGCAGTATAATTAATAACTAATTACTGT | |
| | CGTTGACAGGACACGAGTAACTCGTCTATCTTCTGCAGGCTGCTTACGGT | |
| | TTCGTCCGTGTTGCAGCCGATCATCAGCACATCTAGGTTTCGTCCGGGTG | |
| | TGACCGAAAGGTAAGATGGAGAGCCTTGTCCCTGGTTTCAACGAGAAAAC | |
| | ACACGTCCAACTCAGTTTGCCTGTTTTACAGGTTCGCGACGTGCTCGTAC | |
| | GTGGCTTTGGAGACTCCGTGGAGGAGGTCTTATCAGAGGCACGTCAACAT | |
| | CTTAAAGATGGCACTTGTGGCTTAGTAGAAGTTGAAAAAGGCGTTTTGCC | |
| | TCAACTTGAACAGCCCTATGTGTTCATCAAACGTTCGGATGCTCGAACTG | |
| | CACCTCATGGTCATGTTATGGTTGAGCTGGTAGCAGAACTCGAAGGCATT | |
| | CAGTACGGTCGTAGTGGTGAGACACTTGGTGTCCTTGTCCCTCATGTGGG | |
| | CGAAATACCAGTGGCTTACCGCAAGGTTCTTCTTCGTAAGAACGGTAATA | |
| | AAGGAGCTGGTGGCCATAGTTACGGCGCCGATCTAAAGTCATTTGACTTA | |
| | GGCGACGAGCTTGGCACTGATCCTTATGAAGATTTTCAAGAAAACTGGAA | |
| | CACTAAACATAGCAGTGGTGTTACCCGTGAACTCATGCGTGAGCTTAACG | |
| | GAGGGGCATACACTCGCTATGTCGATAACAACTTCTGTGGCCCTGATGGC | |
| | TACCCTCTTGAGTGCATTAAAGACCTTCTAGCACGTGCTGGTAAAGCTTC | |
| | ATGCACTTTGTCCGAACAACTGGACTTTATTGACACTAAGAGGGGTGTAT | |
| | ACTGCTGCCGTGAACATGAGCATGAAATTGCTTGGTACACGGAACGTTCT | |
| | GAAAAGAGCTATGAATTGCAGACACCTTTTGAAATTAAATTGGCAAAGAA | |
| | ATTTGACACCTTCAATGGGGAATGTCCAAATTTTGTATTTCCCTTAAATT | |
| | CCATAATCAAGACTATTCAACCAAGGGTTGAAAAGAAAAAGCTTGATGGC | |
| | TTTATGGGTAGAATTCGATCTGTCTATCCAGTTGCGTCACCAAATGAATG | |
| | CAACCAAATGTGCCTTTCAACTCTCATGAAGTGTGATCATTGTGGTGAAA | |
| | CTTCATGGCAGACGGGCGATTTTGTTAAAGCCACTTGCGAATTTTGTGGC | |
| | ACTGAGAATTTGACTAAAGAAGGTGCCACTACTTGTGGTTACTTACCCCA | |
| | AAATGCTGTTGTTAAAATTTATTGTCCAGCATGTCACAATTCAGAAGTAG | |
| | GACCTGAGCATAGTCTTGCCGAATACCATAATGAATCTGGCTTGAAAACC | |
| | ATTCTTCGTAAGGGTGGTCGCACTATTGCCTTTGGAGGCTGTGTGTTCTC | |
| | TTATGTTGGTTGCCATAACAAGTGTGCCTATTGGGTTCCACGTGCTAGCG | |
| | CTAACATAGGTTGTAACCATACAGGTGTTGTTGGAGAAGGTTCCGAAGGT | |
| | CTTAATGACAACCTTCTTGAAATACTCCAAAAAGAGAAAGTCAACATCAA | |
| | TATTGTTGGTGACTTTAAACTTAATGAAGAGATCGCCATTATTTTGGCAT | |
| | CTTTTTCTGCTTCCACAAGTGCTTTTGTGGAAACTGTGAAAGGTTTGGAT | |
| | TATAAAGCATTCAAACAAATTGTTGAATCCTGTGGTAATTTTAAAGTTAC | |
| | AAAAGGAAAAGCTAAAAAAGGTGCCTGGAATATTGGTGAACAGAAATCAA | |
| | TACTGAGTCCTCTTTATGCATTTGCATCAGAGGCTGCTCGTGTTGTACGA | |
| | TCAATTTTCTCCCGCACTCTTGAAACTGCTCAAAATTCTGTGCGTGTTTT | |
| | ACAGAAGGCCGCTATAACAATACTAGATGGAATTTCACAGTATTCACTGA | |
| | GACTCATTGATGCTATGATGTTCACATCTGATTTGGCTACTAACAATCTA | |
| | GTTGTAATGGCCTACATTACAGGTGGTGTTGTTCAGTTGACTTCGCAGTG | |
| | GCTAACTAACATCTTTGGCACTGTTTATGAAAAACTCAAACCCGTCCTTG | |
| | ATTGGCTTGAAGAGAAGTTTAAGGAAGGTGTAGAGTTTCTTAGAGACGGT | |
| | TGGGAAATTGTTAAATTTATCTCAACCTGTGCTTGTGAAATTGTCGGTGG | |
| | ACAAATTGTCACCTGTGCAAAGGAAATTAAGGAGAGTGTTCAGACATTCT | |
| | TTAAGCTTGTAAATAAATTTTTGGCTTTGTGTGCTGACTCTATCATTATT | |
| | GGTGGAGCTAAACTTAAAGCCTTGAATTTAGGTGAAACATTTGTCACGCA | |
| | CTCAAAGGGATTGTACAGAAAGTGTGTTAAATCCAGAGAAGAAACTGGCC | |
| | TACTCATGCCTCTAAAAGCCCCAAAAGAAATTATCTTCTTAGAGGGAGAA | |
| | ACACTTCCCACAGAAGTGTTAACAGAGGAAGTTGTCTTGAAAACTGGTGA | |
| | TTTACAACCATTAGAACAACCTACTAGTGAAGCTGTTGAAGCTCCATTGG | |
| | TTGGTACACCAGTTTGTATTAACGGGCTTATGTTGCTCGAAATCAAAGAC | |
| | ACAGAAAAGTACTGTGCCCTTGCACCTAATATGATGGTAACAAACAATAC | |
| | CTTCACACTCAAAGGCGGTGCACCAACAAAGGTTACTTTTGGTGATGACA | |
| | CTGTGATAGAAGTGCAAGGTTACAAGAGTGTGAATATCACTTTTGAACTT | |
| | GATGAAAGGATTGATAAAGTACTTAATGAGAAGTGCTCTGCCTATACAGT | |
| | TGAACTCGGTACAGAAGTAAATGAGTTCGCCTGTGTTGTGGCAGATGCTG | |
| | TCATAAAAACTTTGCAACCAGTATCTGAATTACTTACACCACTGGGCATT | |
| | GATTTAGATGAGTGGAGTATGGCTACATACTACTTATTTGATGAGTCTGG | |
| | TGAGTTTAAATTGGCTTCACATATGTATTGTTCTTTCTACCCTCCAGATG | |
| | AGGATGAAGAAGAAGGTGATTGTGAAGAAGAAGAGTTTGAGCCATCAACT | |
| | CAATATGAGTATGGTACTGAAGATGATTACCAAGGTAAACCTTTGGAATT | |
| | TGGTGCCACTTCTGCTGCTCTTCAACCTGAAGAAGAGCAAGAAGAAGATT | |
| | GGTTAGATGATGATAGTCAACAAACTGTTGGTCAACAAGACGGCAGTGAG | |
| | GACAATCAGACAACTACTATTCAAACAATTGTTGAGGTTCAACCTCAATT | |
| | AGAGATGGAACTTACACCAGTTGTTCAGACTATTGAAGTGAATAGTTTTA | |
| | GTGGTTATTTAAAACTTACTGACAATGTATACATTAAAAATGCAGACATT | |
| | GTGGAAGAAGCTAAAAAGGTAAAACCAACAGTGGTTGTTAATGCAGCCAA | |
| | TGTTTACCTTAAACATGGAGGAGGTGTTGCAGGAGCCTTAAATAAGGCTA | |
| | CTAACAATGCCATGCAAGTTGAATCTGATGATTACATAGCTACTAATGGA | |
| | CCACTTAAAGTGGGTGGTAGTTGTGTTTTAAGCGGACACAATCTTGCTAA | |
| | ACACTGTCTTCATGTTGTCGGCCCAAATGTTAACAAAGGTGAAGACATTC | |
| | AACTTCTTAAGAGTGCTTATGAAAATTTTAATCAGCACGAAGTTCTACTT | |
| | GCACCATTATTATCAGCTGGTATTTTTGGTGCTGACCCTATACATTCTTT | |
| | AAGAGTTTGTGTAGATACTGTTCGCACAAATGTCTACTTAGCTGTCTTTG | |
| | ATAAAAATCTCTATGACAAACTTGTTTCAAGCTTTTTGGAAATGAAGAGT | |
| | GAAAAGCAAGTTGAACAAAAGATCGCTGAGATTCCTAAAGAGGAAGTTAA | |
| | GCCATTTATAACTGAAAGTAAACCTTCAGTTGAACAGAGAAAACAAGATG | |
| | ATAAGAAAATCAAAGCTTGTGTTGAAGAAGTTACAACAACTCTGGAAGAA | |
| | ACTAAGTTCCTCACAGAAAACTTGTTACTTTATATTGACATTAATGGCAA | |
| | TCTTCATCCAGATTCTGCCACTCTTGTTAGTGACATTGACATCACTTTCT | |
| | TAAAGAAAGATGCTCCATATATAGTGGGTGATGTTGTTCAAGAGGGTGTT | |
| | TTAACTGCTGTGGTTATACCTACTAAAAAGGCTGGTGGCACTACTGAAAT | |
| | GCTAGCGAAAGCTTTGAGAAAAGTGCCAACAGACAATTATATAACCACTT | |
| | ACCCGGGTCAGGGTTTAAATGGTTACACTGTAGAGGAGGCAAAGACAGTG | |
| | CTTAAAAAGTGTAAAAGTGCCTTTTACATTCTACCATCTATTATCTCTAA | |
| | TGAGAAGCAAGAAATTCTTGGAACTGTTTCTTGGAATTTGCGAGAAATGC | |
| | TTGCACATGCAGAAGAAACACGCAAATTAATGCCTGTCTGTGTGGAAACT | |
| | AAAGCCATAGTTTCAACTATACAGCGTAAATATAAGGGTATTAAAATACA | |
| | AGAGGGTGTGGTTGATTATGGTGCTAGATTTTACTTTTACACCAGTAAAA | |
| | CAACTGTAGCGTCACTTATCAACACACTTAACGATCTAAATGAAACTCTT | |
| | GTTACAATGCCACTTGGCTATGTAACACATGGCTTAAATTTGGAAGAAGC | |
| | TGCTCGGTATATGAGATCTCTCAAAGTGCCAGCTACAGTTTCTGTTTCTT | |
| | CACCTGATGCTGTTACAGCGTATAATGGTTATCTTACTTCTTCTTCTAAA | |
| | ACACCTGAAGAACATTTTATTGAAACCATCTCACTTGCTGGTTCCTATAA | |
| | AGATTGGTCCTATTCTGGACAATCTACACAACTAGGTATAGAATTTCTTA | |
| | AGAGAGGTGATAAAAGTGTATATTACACTAGTAATCCTACCACATTCCAC | |
| | CTAGATGGTGAAGTTATCACCTTTGACAATCTTAAGACACTTCTTTCTTT | |
| | GAGAGAAGTGAGGACTATTAAGGTGTTTACAACAGTAGACAACATTAACC | |
| | TCCACACGCAAGTTGTGGACATGTCAATGACATATGGACAACAGTTTGGT | |
| | CCAACTTATTTGGATGGAGCTGATGTTACTAAAATAAAACCTCATAATTC | |
| | ACATGAAGGTAAAACATTTTATGTTTTACCTAATGATGACACTCTACGTG | |
| | TTGAGGCTTTTGAGTACTACCACACAACTGATCCTAGTTTTCTGGGTAGG | |
| | TACATGTCAGCATTAAATCACACTAAAAAGTGGAAATACCCACAAGTTAA | |
| | TGGTTTAACTTCTATTAAATGGGCAGATAACAACTGTTATCTTGCCACTG | |
| | CATTGTTAACACTCCAACAAATAGAGTTGAAGTTTAATCCACCTGCTCTA | |
| | CAAGATGCTTATTACAGAGCAAGGGCTGGTGAAGCTGCTAACTTTTGTGC | |
| | ACTTATCTTAGCCTACTGTAATAAGACAGTAGGTGAGTTAGGTGATGTTA | |
| | GAGAAACAATGAGTTACTTGTTTCAACATGCCAATTTAGATTCTTGCAAA | |
| | AGAGTCTTGAACGTGGTGTGTAAAACTTGTGGACAACAGCAGACAACCCT | |
| | TAAGGGTGTAGAAGCTGTTATGTACATGGGCACACTTTCTTATGAACAAT | |
| | TTAAGAAAGGTGTTCAGATACCTTGTACGTGTGGTAAACAAGCTACAAAA | |
| | TATCTAGTACAACAGGAGTCACCTTTTGTTATGATGTCAGCACCACCTGC | |
| | TCAGTATGAACTTAAGCATGGTACATTTACTTGTGCTAGTGAGTACACTG | |
| | GTAATTACCAGTGTGGTCACTATAAACATATAACTTCTAAAGAAACTTTG | |
| | TATTGCATAGACGGTGCTTTACTTACAAAGTCCTCAGAATACAAAGGTCC | |
| | TATTACGGATGTTTTCTACAAAGAAAACAGTTACACAACAACCATAAAAC | |
| | CAGTTACTTATAAATTGGATGGTGTTGTTTGTACAGAAATTGACCCTAAG | |
| | TTGGACAATTATTATAAGAAAGACAATTCTTATTTCACAGAGCAACCAAT | |
| | TGATCTTGTACCAAACCAACCATATCCAAACGCAAGCTTCGATAATTTTA | |
| | AGTTTGTATGTGATAATATCAAATTTGCTGATGATTTAAACCAGTTAACT | |
| | GGTTATAAGAAACCTGCTTCAAGAGAGCTTAAAGTTACATTTTTCCCTGA | |
| | CTTAAATGGTGATGTGGTGGCTATTGATTATAAACACTACACACCCTCTT | |
| | TTAAGAAAGGAGCTAAATTGTTACATAAACCTATTGTTTGGCATGTTAAC | |
| | AATGCAACTAATAAAGCCACGTATAAACCAAATACCTGGTGTATACGTTG | |
| | TCTTTGGAGCACAAAACCAGTTGAAACATCAAATTCGTTTGATGTACTGA | |
| | AGTCAGAGGACGCGCAGGGAATGGATAATCTTGCCTGCGAAGATCTAAAA | |
| | CCAGTCTCTGAAGAAGTAGTGGAAAATCCTACCATACAGAAAGACGTTCT | |
| | TGAGTGTAATGTGAAAACTACCGAAGTTGTAGGAGACATTATACTTAAAC | |
| | CAGCAAATAATAGTTTAAAAATTACAGAAGAGGTTGGCCACACAGATCTA | |
| | ATGGCTGCTTATGTAGACAATTCTAGTCTTACTATTAAGAAACCTAATGA | |
| | ATTATCTAGAGTATTAGGTTTGAAAACCCTTGCTACTCATGGTTTAGCTG | |
| | CTGTTAATAGTGTCCCTTGGGATACTATAGCTAATTATGCTAAGCCTTTT | |
| | CTTAACAAAGTTGTTAGTACAACTACTAACATAGTTACACGGTGTTTAAA | |
| | CCGTGTTTGTACTAATTATATGCCTTATTTCTTTACTTTATTGCTACAAT | |
| | TGTGTACTTTTACTAGAAGTACAAATTCTAGAATTAAAGCATCTATGCCG | |
| | ACTACTATAGCAAAGAATACTGTTAAGAGTGTCGGTAAATTTTGTCTAGA | |
| | GGCTTCATTTAATTATTTGAAGTCACCTAATTTTTCTAAACTGATAAATA | |
| | TTATAATTTGGTTTTTACTATTAAGTGTTTGCCTAGGTTCTTTAATCTAC | |
| | TCAACCGCTGCTTTAGGTGTTTTAATGTCTAATTTAGGCATGCCTTCTTA | |
| | CTGTACTGGTTACAGAGAAGGCTATTTGAACTCTACTAATGTCACTATTG | |
| | CAACCTACTGTACTGGTTCTATACCTTGTAGTGTTTGTCTTAGTGGTTTA | |
| | GATTCTTTAGACACCTATCCTTCTTTAGAAACTATACAAATTACCATTTC | |
| | ATCTTTTAAATGGGATTTAACTGCTTTTGGCTTAGTTGCAGAGTGGTTTT | |
| | TGGCATATATTCTTTTCACTAGGTTTTTCTATGTACTTGGATTGGCTGCA | |
| | ATCATGCAATTGTTTTTCAGCTATTTTGCAGTACATTTTATTAGTAATTC | |
| | TTGGCTTATGTGGTTAATAATTAATCTTGTACAAATGGCCCCGATTTCAG | |
| | CTATGGTTAGAATGTACATCTTCTTTGCATCATTTTATTATGTATGGAAA | |
| | AGTTATGTGCATGTTGTAGACGGTTGTAATTCATCAACTTGTATGATGTG | |
| | TTACAAACGTAATAGAGCAACAAGAGTCGAATGTACAACTATTGTTAATG | |
| | GTGTTAGAAGGTCCTTTTATGTCTATGCTAATGGAGGTAAAGGCTTTTGC | |
| | AAACTACACAATTGGAATTGTGTTAATTGTGATACATTCTGTGCTGGTAG | |
| | TAGATTTATTAGTGATGAAGTTGCGAGAGACTTGTCACTACAGTTTAAAA | |
| | GACCAATAAATCCTACTGACCAGTCTTCTTACATCGTTGATAGTGTTACA | |
| | GTGAAGAATGGTTCCATCCATCTTTACTTTGATAAAGCTGGTCAAAAGAC | |
| | TTATGAAAGACATTCTCTCTCTCATTTTGTTAACTTAGACAACCTGAGAG | |
| | CTAATAACACTAAAGGTTCATTGCCTATTAATGTTATAGTTTTTGATGGT | |
| | AAATCAAAATGTGAAGAATCATCTGCAAAATCAGCGTCTGTTTACTACAG | |
| | TCAGCTTATGTGTCAACCTATACTGTTACTAGATCAGGCATTAGTGTCTG | |
| | ATGTTGGTGATAGTGCGGAAGTTGCAGTTAAAATGTTTGATGCTTACGTT | |
| | AATACGTTTTCATCAACTTTTAACGTACCAATGGAAAAACTCAAAACACT | |
| | AGTTGCAACTGCAGAAGCTGAACTTGCAAAGAATGTGTCCTTAGACAATG | |
| | TCTTATCTACTTTTATTTCAGCAGCTCGGCAAGGGTTTGTTGATTCAGAT | |
| | GTAGAAACTAAAGATGTTGTTGAATGTCTTAAATTGTCACATCAATCTGA | |
| | CATAGAAGTTACTGGCGATAGTTGTAATAACTATATGCTCACCTATAACA | |
| | AAGTTGAAAACATGACACCCCGTGACCTTGGTGCTTGTATTGACTGTAGT | |
| | GCGCGTCATATTAATGCGCAGGTAGCAAAAAGTCACAACATTGCTTTGAT | |
| | ATGGAACGTTAAAGATTTCATGTCATTGTCTGAACAACTACGAAAACAAA | |
| | TACGTAGTGCTGCTAAAAAGAATAACTTACCTTTTAAGTTGACATGTGCA | |
| | ACTACTAGACAAGTTGTTAATGTTGTAACAACAAAGATAGCACTTAAGGG | |
| | TGGTAAAATTGTTAATAATTGGTTGAAGCAGTTAATTAAAGTTACACTTG | |
| | TGTTCCTTTTTGTTGCTGCTATTTTCTATTTAATAACACCTGTTCATGTC | |
| | ATGTCTAAACATACTGACTTTTCAAGTGAAATCATAGGATACAAGGCTAT | |
| | TGATGGTGGTGTCACTCGTGACATAGCATCTACAGATACTTGTTTTGCTA | |
| | ACAAACATGCTGATTTTGACACATGGTTTAGCCAGCGTGGTGGTAGTTAT | |
| | ACTAATGACAAAGCTTGCCCATTGATTGCTGCAGTCATAACAAGAGAAGT | |
| | GGGTTTTGTCGTGCCTGGTTTGCCTGGCACGATATTACGCACAACTAATG | |
| | GTGACTTTTTGCATTTCTTACCTAGAGTTTTTAGTGCAGTTGGTAACATC | |
| | TGTTAGACACCATCAAAACTTATAGAGTACACTGACTTTGCAACATCAGC | |
| | TTGTGTTTTGGCTGCTGAATGTACAATTTTTAAAGATGCTTCTGGTAAGC | |
| | CAGTACCATATTGTTATGATACCAATGTACTAGAAGGTTCTGTTGCTTAT | |
| | GAAAGTTTACGCCCTGACACACGTTATGTGCTCATGGATGGCTCTATTAT | |
| | TCAATTTCCTAACACCTACCTTGAAGGTTCTGTTAGAGTGGTAACAACTT | |
| | TTGATTCTGAGTACTGTAGGCACGGCACTTGTGAAAGATCAGAAGCTGGT | |
| | GTTTGTGTATCTACTAGTGGTAGATGGGTACTTAACAATGATTATTACAG | |
| | ATCTTTACCAGGAGTTTTCTGTGGTGTAGATGCTGTAAATTTACTTACTA | |
| | ATATGTTTACACCACTAATTCAACCTATTGGTGCTTTGGACATATCAGCA | |
| | TCTATAGTAGCTGGTGGTATTGTAGCTATCGTAGTAACATGCCTTGCCTA | |
| | CTATTTTATGAGGTTTAGAAGAGCTTTTGGTGAATACAGTCATGTAGTTG | |
| | CCTTTAATACTTTACTATTCCTTATGTCATTCACTGTACTCTGTTTAACA | |
| | CCAGTTTACTCATTCTTACCTGGTGTTTATTCTGTTATTTACTTGTACTT | |
| | GACATTTTATCTTACTAATGATGTTTCTTTTTTAGCACATATTCAGTGGA | |
| | TGGTTATGTTCACACCTTTAGTACCTTTCTGGATAACAATTGCTTATATC | |
| | ATTTGTATTTCCACAAAGCATTTCTATTGGTTCTTTAGTAATTACCTAAA | |
| | GAGACGTGTAGTCTTTAATGGTGTTTCCTTTAGTACTTTTGAAGAAGCTG | |
| | CGCTGTGCACCTTTTTGTTAAATAAAGAAATGTATCTAAAGTTGCGTAGT | |
| | GATGTGCTATTACCTCTTACGCAATATAATAGATACTTAGCTCTTTATAA | |
| | TAAGTACAAGTATTTTAGTGGAGCAATGGATACAACTAGCTACAGAGAAG | |
| | CTGCTTGTTGTCATCTCGCAAAGGCTCTCAATGACTTCAGTAACTCAGGT | |
| | TCTGATGTTCTTTACCAACCACCACAAACCTCTATCACCTCAGCTGTTTT | |
| | GCAGAGTGGTTTTAGAAAAATGGCATTCCCATCTGGTAAAGTTGAGGGTT | |
| | GTATGGTACAAGTAACTTGTGGTACAACTACACTTAACGGTCTTTGGCTT | |
| | GATGACGTAGTTTACTGTCCAAGACATGTGATCTGCACCTCTGAAGACAT | |
| | GCTTAACCCTAATTATGAAGATTTACTCATTCGTAAGTCTAATCATAATT | |
| | TCTTGGTACAGGCTGGTAATGTTCAACTCAGGGTTATTGGACATTCTATG | |
| | CAAAATTGTGTACTTAAGCTTAAGGTTGATACAGCCAATCCTAAGACACC | |
| | TAAGTATAAGTTTGTTCGCATTCAACCAGGACAGACTTTTTCAGTGTTAG | |
| | CTTGTTACAATGGTTCACCATCTGGTGTTTACCAATGTGCTATGAGGCCC | |
| | AATTTCACTATTAAGGGTTCATTCCTTAATGGTTCATGTGGTAGTGTTGG | |
| | TTTTAACATAGATTATGACTGTGTCTCTTTTTGTTACATGCACCATATGG | |
| | AATTACCAACTGGAGTTCATGCTGGCACAGACTTAGAAGGTAACTTTTAT | |
| | GGACCTTTTGTTGACAGGCAAACAGCACAAGCAGCTGGTACGGACACAAC | |
| | TATTACAGTTAATGTTTTAGCTTGGTTGTACGCTGCTGTTATAAATGGAG | |
| | ACAGGTGGTTTCTCAATCGATTTACCACAACTCTTAATGACTTTAACCTT | |
| | GTGGCTATGAAGTACAATTATGAACCTCTAACACAAGACCATGTTGACAT | |
| | ACTAGGACCTCTTTCTGCTCAAACTGGAATTGCCGTTTTAGATATGTGTG | |
| | CTTCATTAAAAGAATTACTGCAAAATGGTATGAATGGACGTACCATATTG | |
| | GGTAGTGCTTTATTAGAAGATGAATTTACACCTTTTGATGTTGTTAGACA | |
| | ATGCTCAGGTGTTACTTTCCAAAGTGCAGTGAAAAGAACAATCAAGGGTA | |
| | CACACCACTGGTTGTTACTCACAATTTTGACTTCACTTTTAGTTTTAGTC | |
| | CAGAGTACTCAATGGTCTTTGTTCTTTTTTTTGTATGAAAATGCCTTTTT | |
| | ACCTTTTGCTATGGGTATTATTGCTATGTCTGCTTTTGCAATGATGTTTG | |
| | TCAAACATAAGCATGCATTTCTCTGTTTGTTTTTGTTACCTTCTCTTGCC | |
| | ACTGTAGCTTATTTTAATATGGTCTATATGCCTGCTAGTTGGGTGATGCG | |
| | TATTATGACATGGTTGGATATGGTTGATACTAGTTTGTCTGGTTTTAAGC | |
| | TAAAAGACTGTGTTATGTATGCATCAGCTGTAGTGTTACTAATCCTTATG | |
| | ACAGCAAGAACTGTGTATGATGATGGTGCTAGGAGAGTGTGGACACTTAT | |
| | GAATGTCTTGACACTCGTTTATAAAGTTTATTATGGTAATGCTTTAGATC | |
| | AAGCCATTTCCATGTGGGCTCTTATAATCTCTGTTACTTCTAACTACTCA | |
| | GGTGTAGTTACAACTGTCATGTTTTTGGCCAGAGGTATTGTTTTTATGTG | |
| | TGTTGAGTATTGCCCTATTTTCTTCATAACTGGTAATACACTTCAGTGTA | |
| | TAATGCTAGTTTATTGTTTCTTAGGCTATTTTTGTACTTGTTACTTTGGC | |
| | CTCTTTTGTTTACTCAACCGCTACTTTAGACTGACTCTTGGTGTTTATGA | |
| | TTACTTAGTTTCTACACAGGAGTTTAGATATATGAATTCACAGGGACTAC | |
| | TCCCACCCAAGAATAGCATAGATGCCTTCAAACTCAACATTAAATTGTTG | |
| | GGTGTTGGTGGCAAACCTTGTATCAAAGTAGCCACTGTACAGTCTAAAAT | |
| | GTCAGATGTAAAGTGCACATCAGTAGTCTTACTCTCAGTTTTGCAACAAC | |
| | TCAGAGTAGAATCATCATCTAAATTGTGGGCTCAATGTGTCCAGTTACAC | |
| | AATGACATTCTCTTAGCTAAAGATACTACTGAAGCCTTTGAAAAAATGGT | |
| | TTCACTACTTTCTGTTTTGCTTTCCATGCAGGGTGCTGTAGACATAAACA | |
| | AGCTTTGTGAAGAAATGCTGGACAACAGGGCAACCTTACAAGCTATAGCC | |
| | TCAGAGTTTAGTTCCCTTCCATCATATGCAGCTTTTGCTACTGCTCAAGA | |
| | AGCTTATGAGCAGGCTGTTGCTAATGGTGATTCTGAAGTTGTTCTTAAAA | |
| | AGTTGAAGAAGTCTTTGAATGTGGCTAAATCTGAATTTGACCGTGATGCA | |
| | GCCATGCAACGTAAGTTGGAAAAGATGGCTGATCAAGCTATGACCCAAAT | |
| | GTATAAACAGGCTAGATCTGAGGACAAGAGGGCAAAAGTTACTAGTGCTA | |
| | TGCAGACAATGCTTTTCACTATGCTTAGAAAGTTGGATAATGATGCACTC | |
| | AACAACATTATCAACAATGCAAGAGATGGTTGTGTTCCCTTGAACATAAT | |
| | ACCTCTTACAACAGCAGCCAAACTAATGGTTGTCATACCAGACTATAACA | |
| | CATATAAAAATACGTGTGATGGTACAACATTTACTTATGCATCAGCATTG | |
| | TGGGAAATCCAACAGGTTGTAGATGCAGATAGTAAAATTGTTCAACTTAG | |
| | TGAAATTAGTATGGACAATTCACCTAATTTAGCATGGCCTCTTATTGTAA | |
| | CAGCTTTAAGGGCCAATTCTGCTGTCAAATTACAGAATAATGAGCTTAGT | |
| | CCTGTTGCACTACGACAGATGTCTTGTGCTGCCGGTACTACACAAACTGC | |
| | TTGCACTGATGACAATGCGTTAGCTTACTACAACACAACAAAGGGAGGTA | |
| | GGTTTGTACTTGCACTGTTATCCGATTTACAGGATTTGAAATGGGCTAGA | |
| | TTCCCTAAGAGTGATGGAACTGGTACTATCTATACAGAACTGGAACCACC | |
| | TTGTAGGTTTGTTACAGACACACCTAAAGGTCCTAAAGTGAAGTATTTAT | |
| | ACTTTATTAAAGGATTAAACAACCTAAATAGAGGTATGGTACTTGGTAGT | |
| | TTAGCTGCCACAGTACGTCTACAAGCTGGTAATGCAACAGAAGTGCCTGC | |
| | CAATTCAACTGTATTATCTTTCTGTGCTTTTGCTGTAGATGCTGCTAAAG | |
| | CTTACAAAGATTATCTAGCTAGTGGGGGACAACCAATCACTAATTGTGTT | |
| | AAGATGTTGTGTACACACACTGGTACTGGTCAGGCAATAACAGTTACACC | |
| | GGAAGCCAATATGGATCAAGAATCCTTTGGTGGTGCATCGTGTTGTCTGT | |
| | ACTGCCGTTGCCACATAGATCATCCAAATCCTAAAGGATTTTGTGACTTA | |
| | AAAGGTAAGTATGTACAAATACCTACAACTTGTGCTAATGACCCTGTGGG | |
| | TTTTACACTTAAAAACACAGTCTGTACCGTCTGCGGTATGTGGAAAGGTT | |
| | ATGGCTGTAGTTGTGATCAACTCCGCGAACCCATGCTTCAGTCAGCTGAT | |
| | GCACAATCGTTTTTAAACGGGTTTGCGGTGTAAGTGCAGCCCGTCTTACA | |
| | CCGTGCGGCACAGGCACTAGTACTGATGTCGTATACAGGGCTTTTGACAT | |
| | CTACAATGATAAAGTAGCTGGTTTTGCTAAATTCCTAAAAACTAATTGTT | |
| | GTCGCTTCCAAGAAAAGGACGAAGATGACAATTTAATTGATTCTTACTTT | |
| | GTAGTTAAGAGACACACTTTCTCTAACTAGCAACATGAAGAAACAATTTA | |
| | TAATTTACTTAAGGATTGTCCAGCTGTTGCTAAACATGACTTCTTTAAGT | |
| | TTAGAATAGACGGTGACATGGTACCACATATATCACGTCAACGTCTTACT | |
| | AAATACACAATGGCAGACCTCGTCTATGCTTTAAGGCATTTTGATGAAGG | |
| | TAATTGTGACACATTAAAAGAAATACTTGTCACATACAATTGTTGTGATG | |
| | ATGATTATTTCAATAAAAAGGACTGGTATGATTTTGTAGAAAACCCAGAT | |
| | ATATTACGCGTATACGCCAACTTAGGTGAACGTGTACGCCAAGCTTTGTT | |
| | AAAAACAGTACAATTCTGTGATGCCATGCGAAATGCTGGTATTGTTGGTG | |
| | TACTGACATTAGATAATCAAGATCTCAATGGTAACTGGTATGATTTCGGT | |
| | GATTTCATACAAACCACGCCAGGTAGTGGAGTTCCTGTTGTAGATTCTTA | |
| | TTATTCATTGTTAATGCCTATATTAACCTTGACCAGGGCTTTAACTGCAG | |
| | AGTCACATGTTGACACTGACTTAACAAAGCCTTACATTAAGTGGGATTTG | |
| | TTAAAATATGACTTCACGGAAGAGAGGTTAAAACTCTTTGACCGTTATTT | |
| | TAAATATTGGGATCAGACATACCACCCAAATTGTGTTAACTGTTTGGATG | |
| | ACAGATGCATTCTGCATTGTGCAAACTTTAATGTTTTATTCTCTACAGTG | |
| | TTCCCACCTACAAGTTTTGGACCACTAGTGAGAAAAATATTTGTTGATGG | |
| | TGTTCCATTTGTAGTTTCAACTGGATACCACTTCAGAGAGCTAGGTGTTG | |
| | TAGATAATCAGGATGTAAACTTACATAGCTCTAGACTTAGTTTTAAGGAA | |
| | TTACTTGTGTATGCTGCTGACCCTGCTATGCACGCTGCTTCTGGTAATCT | |
| | ATTACTAGATAAACGCACTACGTGCTTTTCAGTAGCTGCACTTACTAACA | |
| | ATGTTGCTTTTCAAACTGTCAAACCCGGTAATTTTAACAAAGACTTCTAT | |
| | GACTTTGCTGTGTCTAAGGGTTTCTTTAAGGAAGGAAGTTCTGTTGAATT | |
| | AAAACACTTCTTCTTTGCTCAGGATGGTAATGCTGCTATCAGCGATTATG | |
| | ACTACTATCGTTATAATCTACCAACAATGTGTGATATCAGACAACTACTA | |
| | TTTGTAGTTGAAGTTGTTGATAAGTACTTTGATTGTTACGATGGTGGCTG | |
| | TATTAATGCTAACCAAGTCATCGTCAACAACCTAGACAAATCAGCTGGTT | |
| | TTCCATTTAATAAATGGGGTAAGGCTAGACTTTATTATGATTCAATGAGT | |
| | TATGAGGATCAAGATGCACTTTTCGCATATACAAAACGTAATGTCATCCC | |
| | TAGTATAACTCAAATGAATCTTAAGTATGCCATTAGTGCAAAGAATAGAG | |
| | CTCGCACCGTAGCTGGTGTCTCTATCTGTAGTACTATGACCAATAGACAG | |
| | TTTCATCAAAAATTATTGAAATCAATAGCCGCCACTAGAGGAGCTACTGT | |
| | AGTAATTGGAACAAGCAAATTCTATGGTGGTTGGCACAACATGTTAAAAA | |
| | CTGTTTATAGTGATGTAGAAAACCCTCACCTTATGGGTTGGGATTATCCT | |
| | AAATGTGATAGAGCCATGCCTAACATGCTTAGAATTATGGCCTCACTTGT | |
| | TCTTGCTCGCAAACATACAACGTGTTGTAGCTTGTCACACCGTTTCTATA | |
| | GATTAGCTAATGAGTGTGCTCAAGTATTGAGTGAAATGGTCATGTGTGGC | |
| | GGTTCACTATATGTTAAACCAGGTGGAACCTCATCAGGAGATGCCACAAC | |
| | TGCTTATGCTAATAGTGTTTTTAACATTTGTCAAGCTGTCACGGCCAATG | |
| | TTAATGCACTTTTATCTACTGATGGTAACAAAATTGCCGATAAGTATGTC | |
| | CGCAATTTACAACACAGACTTTATGAGTGTCTCTATAGAAATAGAGATGT | |
| | TGACACAGACTTTGTGAATGAGTTTTACGCATATTTGCGTAAACATTTCT | |
| | CAATGATGATACTCTCTGACGATGCTGTTGTGTGTTTCAATAGCACTTAT | |
| | GCATCTCAAGGTCTAGTGGCTAGCATAAAGAACTTTAAGTCAGTTCTTTA | |
| | TTATCAAAACAATGTTTTTATGTCTGAAGCAAAATGTTGGACTGAGACTG | |
| | ACCTTACTAAAGGACCTCATGAATTTTGCTCTCAACATACAATGCTAGTT | |
| | AAACAGGGTGATGATTATGTGTACCTTCCTTACCCAGATCCATCAAGAAT | |
| | CCTAGGGGCCGGCTGTTTTGTAGATGATATCGTAAAAACAGATGGTACAC | |
| | TTATGATTGAACGGTTCGTGTCTTTAGCTATAGATGCTTACCCACTTACT | |
| | AAACATCCTAATCAGGAGTATGCTGATGTCTTTCATTTGTACTTACAATA | |
| | CATAAGAAAGCTACATGATGAGTTAACAGGACACATGTTAGAGATGTATT | |
| | CTGTTATGCTTACTAATGATAACACTTCAAGGTATTGGGAACCTGAGTTT | |
| | TATGAGGCTATGTACACACCGCATACAGTCTTACAGGCTGTTGGGGCTTG | |
| | TGTTCTTTGCAATTCACAGACTTCATTAAGATGTGGTGCTTGCATACGTA | |
| | GACCATTCTTATGTTGTAAATGCTGTTACGACCATGTCATATCAACATCA | |
| | CATAAATTAGTCTTGTCTGTTAATCCGTATGTTTGCAATGCTCCAGGTTG | |
| | TGATGTCACAGATGTGACTCAACTTTACTTAGGAGGTATGAGCTATTATT | |
| | GTAAATCACATAAACCACCCATTAGTTTTCCATTGTGTGCTAATGGACAA | |
| | GTTTTTGGTTTATATAAAAATACATGTGTTGGTAGCGATAATGTTACTGA | |
| | CTTTAATGCAATTGCAACATGTGACTGGACAAATGCTGGTGATTACATTT | |
| | TAGCTAACACCTGTACTGAAAGACTCAAGCTTTTTGCAGCAGAAACGCTC | |
| | AAAGCTACTGAGGAGACATTTAAACTGTCTTATGGTATTGCTACTGTACG | |
| | TGAAGTGCTGTCTGACAGAGAATTACATCTTTCATGGGAAGTTGGTAAAC | |
| | CTAGACCACCACTTAACCGAAATTATGTCTTTACTGGTTATCGTGTAACT | |
| | AAAAACAGTAAAGTACAAATAGGAGAGTACACCTTTGAAAAAGGTGACTA | |
| | TGGTGATGCTGTTGTTTACCGAGGTACAACAACTTACAAATTAAATGTTG | |
| | GTGATTATTTTGTGCTGACATCACATACAGTAATGCCATTAAGTGCACCT | |
| | ACACTAGTGCCACAAGAGCACTATGTTAGAATTACTGGCTTATACCCAAC | |
| | ACTCAATATCTCAGATGAGTTTTCTAGCAATGTTGCAAATTATCAAAAGG | |
| | TTGGTATGCAAAAGTATTCTACACTCCAGGGACCACCTGGTACTGGTAAG | |
| | AGTCATTTTGCTATTGGCCTAGCTCTCTACTACCCTTCTGCTCGCATAGT | |
| | GTATACAGCTTGCTCTCATGCCGCTGTTGATGCACTATGTGAGAAGGCAT | |
| | TAAAATATTTGCCTATAGATAAATGTAGTAGAATTATACCTGCACGTGCT | |
| | CGTGTAGAGTGTTTTGATAAATTCAAAGTGAATTCAACATTAGAACAGTA | |
| | TGTCTTTTGTACTGTAAATGCATTGCCTGAGACGACAGCAGATATAGTTG | |
| | TCTTTGATGAAATTTCAATGGCCACAAATTATGATTTGAGTGTTGTCAAT | |
| | GCCAGATTACGTGCTAAGCACTATGTGTACATTGGCGACCCTGCTCAATT | |
| | ACCTGCACCACGCACATTGCTAACTAAGGGCACACTAGAACCAGAATATT | |
| | TCAATTCAGTGTGTAGACTTATGAAAACTATAGGTCCAGACATGTTCCTC | |
| | GGAACTTGTCGGCGTTGTCCTGCTGAAATTGTTGACACTGTGAGTGCTTT | |
| | GGTTTATGATAATAAGCTTAAAGCACATAAAGACAAATCAGCTCAATGCT | |
| | TTAAAATGTTTTATAAGGGTGTTATCACGCATGATGTTTCATCTGCAATT | |
| | AACAGGCCACAAATAGGCGTGGTAAGAGAATTCCTTACACGTAACCCTGC | |
| | TTGGAGAAAAGCTGTCTTTATTTCACCTTATAATTCACAGAATGCTGTAG | |
| | CCTCAAAGATTTTGGGACTACCAACTCAAACTGTTGATTCATCACAGGGC | |
| | TCAGAATATGACTATGTCATATTCACTCAAACCACTGAAACAGCTCACTC | |
| | TTGTAATGTAAACAGATTTAATGTTGCTATTACCAGAGCAAAAGTAGGCA | |
| | TACTTTGCATAATGTCTGATAGAGACCTTTATGACAAGTTGCAATTTACA | |
| | AGTCTTGAAATTCCACGTAGGAATGTGGCAACTTTACAAGCTGAAAATGT | |
| | AACAGGACTCTTTAAAGATTGTAGTAAGGTAATCACTGGGTTACATCCTA | |
| | CACAGGCACCTACACACCTCAGTGTTGACACTAAATTCAAAACTGAAGGT | |
| | TTATGTGTTGACATACCTGGCATACCTAAGGACATGACCTATAGAAGACT | |
| | CATCTCTATGATGGGTTTTAAAATGAATTATCAAGTTAATGGTTACCCTA | |
| | ACATGTTTATCACCCGCGAAGAAGCTATAAGACATGTACGTGCATGGATT | |
| | GGCTTCGATGTCGAGGGGTGTCATGCTACTAGAGAAGCTGTTGGTACCAA | |
| | TTTACCTTTACAGCTAGGTTTTTCTACAGGTGTTAACCTAGTTGCTGTAC | |
| | CTACAGGTTATGTTGATACACCTAATAATACAGATTTTTCCAGAGTTAGT | |
| | GCTAAACCACCGCCTGGAGATCAATTTAAACACCTCATACCACTTATGTA | |
| | CAAAGGACTTCCTTGGAATGTAGTGCGTATAAAGATTGTACAAATGTTAA | |
| | GTGACACACTTAAAAATCTCTCTGACAGAGTCGTATTTGTCTTATGGGCA | |
| | CATGGCTTTGAGTTGACATCTATGAAGTATTTTGTGAAAATAGGACCTGA | |
| | GCGCACCTGTTGTCTATGTGATAGACGTGCCACATGCTTTTCCACTGCTT | |
| | CAGACACTTATGCCTGTTGGCATCATTCTATTGGATTTGATTACGTCTAT | |
| | AATCCGTTTATGATTGATGTTCAACAATGGGGTTTTACAGGTAACCTACA | |
| | AAGCAACCATGATCTGTATTGTCAAGTCCATGGTAATGCACATGTAGCTA | |
| | GTTGTGATGCAATCATGACTAGGTGTCTAGCTGTCCACGAGTGCTTTGTT | |
| | AAGCGTGTTGACTGGACTATTGAATATCCTATAATTGGTGATGAACTGAA | |
| | GATTAATGCGGCTTGTAGAAAGGTTCAACACATGGTTGTTAAAGCTGCAT | |
| | TATTAGCAGACAAATTCCCAGTTCTTCACGACATTGGTAACCCTAAAGCT | |
| | ATTAAGTGTGTACCTCAAGCTGATGTAGAATGGAAGTTCTATGATGCACA | |
| | GCCTTGTAGTGACAAAGCTTATAAAATAGAAGAATTATTCTATTCTTATG | |
| | CCACACATTCTGACAAATTCACAGATGGTGTATGCCTATTTTGGAATTGC | |
| | AATGTCGATAGATATCCTGCTAATTCCATTGTTTGTAGATTTGACACTAG | |
| | AGTGCTATCTAACCTTAACTTGCCTGGTTGTGATGGTGGCAGTTTGTATG | |
| | TAAATAAACATGCATTCCACACACCAGCTTTTGATAAAAGTGCTTTTGTT | |
| | AATTTAAAACAATTACCATTTTTCTATTACTCTGACAGTCCATGTGAGTC | |
| | TCATGGAAAACAAGTAGTGTCAGATATAGATTATGTACCACTAAAGTCTG | |
| | CTACGTGTATAACACGTTGCAATTTAGGTGGTGCTGTCTGTAGACATCAT | |
| | GCTAATGAGTACAGATTGTATCTCGATGCTTATAACATGATGATCTCAGC | |
| | TGGCTTTAGCTTGTGGGTTTACAAACAATTTGATACTTATAACCTCTGGA | |
| | ACACTTTTACAAGACTTCAGAGTTTAGAAAATGTGGCTTTTAATGTTGTA | |
| | AATAAGGGACACTTTGATGGACAACAGGGTGAAGTACCAGTTTCTATCAT | |
| | TAATAACACTGTTTACACAAAAGTTGATGGTGTTGATGTAGAATTGTTTG | |
| | AAAATAAAACAACATTACCTGTTAATGTAGCATTTGAGCTTTGGGCTAAG | |
| | CGCAACATTAAACCAGTACCAGAGGTGAAAATACTCAATAATTTGGGTGT | |
| | GGACATTGCTGCTAATACTGTGATCTGGGACTACAAAAGAGATGCTCCAG | |
| | CACATATATCTACTATTGGTGTTTGTTCTATGACTGACATAGCCAAGAAA | |
| | CCAACTGAAACGATTTGTGCACCACTCACTGTCTTTTTTGATGGTAGAGT | |
| | TGATGGTCAAGTAGACTTATTTAGAAATGCCCGTAATGGTGTTCTTATTA | |
| | CAGAAGGTAGTGTTAAAGGTTTACAACCATCTGTAGGTCCCAAACAAGCT | |
| | AGTCTTAATGGAGTCACATTAATTGGAGAAGCCGTAAAAACACAGTTCAA | |
| | TTATTATAAGAAAGTTGATGGTGTTGTCCAACAATTACCTGAAACTTACT | |
| | TTACTCAGAGTAGAAATTTACAAGAATTTAAACCCAGGAGTCAAATGGAA | |
| | ATTGATTTCTTAGAATTAGCTATGGATGAATTCATTGAACGGTATAAATT | |
| | AGAAGGCTATGCCTTCGAACATATCGTTTATGGAGATTTTAGTCATAGTC | |
| | AGTTAGGTGGTTTACATCTACTGATTGGACTAGCTAAACGTTTTAAGGAA | |
| | TCACCTTTTGAATTAGAAGATTTTATTCCTATGGACAGTACAGTTAAAAA | |
| | CTATTTCATAACAGATGCGCAAACAGGTTCATCTAAGTGTGTGTGTTCTG | |
| | TTATTGATTTATTACTTGATGATTTTGTTGAAATAATAAAATCCCAAGAT | |
| | TTATCTGTAGTTTCTAAGGTTGTCAAAGTGACTATTGACTATACAGAAAT | |
| | TTCATTTATGCTTTGGTGTAAAGATGGCCATGTAGAAACATTTTACCCAA | |
| | AATTACAATCTAGTCAAGCGTGGCAACCGGGTGTTGCTATGCCTAATCTT | |
| | TACAAAATGCAAAGAATGCTATTAGAAAAGTGTGACCTTCAAAATTATGG | |
| | TGATAGTGCAACATTACCTAAAGGCATAATGATGAATGTCGCAAAATATA | |
| | CTCAACTGTGTCAATATTTAAACACATTAACATTAGCTGTACCCTATAAT | |
| | ATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATT | |
| | CAGATCTTAATGACTTTGTCTCTGATGCAGATTCAACTTTGATTGGTGAT | |
| | TGTGCAACTGTACATACAGCTAATAAATGGGATCTCATTATTAGTGATAT | |
| | GTACGACCCTAAGACTAAAAATGTTACAAAAGAAAATGACTCTAAAGAGG | |
| | GTTTTTTCACTTACATTTGTGGGTTTATACAACAAAAGCTAGCTCTTGGA | |
| | GGTTCCGTGGCTATAAAGATAACAGAACATTCTTGGAATGCTGATCTTTA | |
| | TAAGCTCATGGGACACTTCGCATGGTGGACAGCCTTTGTTACTAATGTGA | |
| | ATGCGTCATCATCTGAAGCATTTTTAATTGGATGTAATTATCTTGGCAAA | |
| | CCACGCGAACAAATAGATGGTTATGTCATGCATGCAAATTACATATTTTG | |
| | GAGGAATACAAATCCAATTCAGTTGTCTTCCTATTCTTTATTTGACATGA | |
| | GTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTAT | |
| | AATTAGAGAAAACAACAGAGTTGTTATTTCTAGTGATGTTCTTGTTAACA | |
| | ACTAAACGAACAATGTTTGTTTTTCTTGTTTTATTGCCACTAGTCTCTAG | |
| | TCAGTGTGTTAATCTTACAACCAGAACTCAATTACCCCCTGCATACACTA | |
| | ATTCTTTCACACGTGGTGTTTATTACCCTGACAAAGTTTTCAGATCCTCA | |
| | GTTTTACATTCAACTCAGGACTTGTTCTTACCTTTCTTTTCCAATGTTAC | |
| | TTGGTTCCATGCTATACATGTCTCTGGGACCAATGGTACTAAGAGGTTTG | |
| | ATAACCCTGTCCTACCATTTAATGATGGTGTTTATTTTGCTTCCACTGAG | |
| | AAGTCTAACATAATAAGAGGCTGGATTTTTGGTACTACTTTAGATTCGAA | |
| | GACCCAGTCCCTACTTATTGTTAATAACGCTACTAATGTTGTTATTAAAG | |
| | TCTGTGAATTTCAATTTTGTAATGATCCATTTTTGGGTGTTTATTACCAC | |
| | AAAAACAACAAAAGTTGGATGGAAAGTGAGTTCAGAGTTTATTCTAGTGC | |
| | GAATAATTGCACTTTTGAATATGTCTCTCAGCCTTTTCTTATGGACCTTG | |
| | AAGGAAAACAGGGTAATTTCAAAAATCTTAGGGAATTTGTGTTTAAGAAT | |
| | ATTGATGGTTATTTTAAAATATATTCTAAGCACACGCCTATTAATTTAGT | |
| | GCGTGATCTCCCTCAGGGTTTTTCGGCTTTAGAACCATTGGTAGATTTGC | |
| | CAATAGGTATTAACATCACTAGGTTTCAAACTTTACTTGCTTTACATAGA | |
| | AGTTATTTGACTCCTGGTGATTCTTCTTCAGGTTGGACAGCTGGTGCTGC | |
| | AGCTTATTATGTGGGTTATCTTCAACCTAGGACTTTTCTATTAAAATATA | |
| | ATGAAAATGGAACCATTACAGATGCTGTAGACTGTGCACTTGACCCTCTC | |
| | TCAGAAACAAAGTGTACGTTGAAATCCTTCACTGTAGAAAAAGGAATCTA | |
| | TCAAACTTCTAACTTTAGAGTCCAACCAACAGAATCTATTGTTAGATTTC | |
| | CTAATATTACAAACTTGTGCCCTTTTGGTGAAGTTTTTAACGCCACCAGA | |
| | TTTGCATCTGTTTATGCTTGGAACAGGAAGAGAATCAGCAACTGTGTTGC | |
| | TGATTATTCTGTCCTATATAATTCCGCATCATTTTCCACTTTTAAGTGTT | |
| | ATGGAGTGTCTCCTACTAAATTAAATGATCTCTGCTTTACTAATGTCTAT | |
| | GCAGATTCATTTGTAATTAGAGGTGATGAAGTCAGACAAATCGCTCCAGG | |
| | GCAAACTGGAAAGATTGCTGATTATAATTATAAATTACCAGATGATTTTA | |
| | CAGGCTGCGTTATAGCTTGGAATTCTAACAATCTTGATTCTAAGGTTGGT | |
| | GGTAATTATAATTACCTGTATAGATTGTTTAGGAAGTCTAATCTCAAACC | |
| | TTTTGAGAGAGATATTTCAACTGAAATCTATCAGGCCGGTAGCACACCTT | |
| | GTAATGGTGTTGAAGGTTTTAATTGTTACTTTCCTTTACAATCATATGGT | |
| | TTCCAACCCACTAATGGTGTTGGTTACCAACCATACAGAGTAGTAGTACT | |
| | TTCTTTTGAACTTCTACATGCACCAGCAACTGTTTGTGGACCTAAAAAGT | |
| | CTACTAATTTGGTTAAAAACAAATGTGTCAATTTCAACTTCAATGGTTTA | |
| | ACAGGCACAGGTGTTCTTACTGAGTCTAACAAAAAGTTTCTGCCTTTCCA | |
| | ACAATTTGGCAGAGACATTGCTGACACTACTGATGCTGTCCGTGATCCAC | |
| | AGACACTTGAGATTCTTGACATTACACCATGTTCTTTTGGTGGTGTCAGT | |
| | GTTATAACACCAGGAACAAATACTTCTAACCAGGTTGCTGTTCTTTATCA | |
| | GGATGTTAACTGCACAGAAGTCCCTGTTGCTATTCATGCAGATCAACTTA | |
| | CTCCTACTTGGCGTGTTTATTCTACAGGTTCTAATGTTTTTCAAACACGT | |
| | GCAGGCTGTTTAATAGGGGCTGAACATGTCAACAACTCATATGAGTGTGA | |
| | CATACCCATTGGTGCAGGTATATGCGCTAGTTATCAGACTCAGACTAATT | |
| | CTCCTCGGCGGGCACGTAGTGTAGCTAGTCAATCCATCATTGCCTACACT | |
| | ATGTCACTTGGTGCAGAAAATTCAGTTGCTTACTCTAATAACTCTATTGC | |
| | CATACCCACAAATTTTACTATTAGTGTTACCACAGAAATTCTACCAGTGT | |
| | CTATGACCAAGACATCAGTAGATTGTACAATGTACATTTGTGGTGATTCA | |
| | ACTGAATGCAGCAATCTTTTGTTGCAATATGGCAGTTTTTGTACACAATT | |
| | AAACCGTGCTTTAACTGGAATAGCTGTTGAACAAGACAAAAACACCCAAG | |
| | AAGTTTTTGCACAAGTCAAACAAATTTACAAAACACCACCAATTAAAGAT | |
| | TTTGGTGGTTTTAATTTTTCACAAATATTACCAGATCCATCAAAACCAAG | |
| | CAAGAGGTCATTTATTGAAGATCTACTTTTCAACAAAGTGACACTTGCAG | |
| | ATGCTGGCTTCATCAAACAATATGGTGATTGCCTTGGTGATATTGCTGCT | |
| | AGAGACCTCATTTGTGCACAAAAGTTTAACGGCCTTACTGTTTTGCCACC | |
| | TTTGCTCACAGATGAAATGATTGCTCAATACACTTCTGCACTGTTAGCGG | |
| | GTACAATCACTTCTGGTTGGACCTTTGGTGCAGGTGCTGCATTACAAATA | |
| | CCATTTGCTATGCAAATGGCTTATAGGTTTAATGGTATTGGAGTTACACA | |
| | GAATGTTCTCTATGAGAACCAAAAATTGATTGCCAACCAATTTAATAGTG | |
| | CTATTGGCAAAATTCAAGACTCACTTTCTTCCACAGCAAGTGCACTTGGA | |
| | AAACTTCAAGATGTGGTCAACCAAAATGCACAAGCTTTAAACACGCTTGT | |
| | TAAACAACTTAGCTCCAATTTTGGTGCAATTTCAAGTGTTTTAAATGATA | |
| | TCCTTTCACGTCTTGACAAAGTTGAGGCTGAAGTGCAAATTGATAGGTTG | |
| | ATCACAGGCAGACTTCAAAGTTTGCAGACATATGTGACTCAACAATTAAT | |
| | TAGAGCTGCAGAAATCAGAGCTTCTGCTAATCTTGCTGCTACTAAAATGT | |
| | CAGAGTGTGTACTTGGACAATCAAAAAGAGTTGATTTTTGTGGAAAGGGC | |
| | TATCATCTTATGTCCTTCCCTCAGTCAGCACCTCATGGTGTAGTCTTCTT | |
| | GCATGTGACTTATGTCCCTGCACAAGAAAAGAACTTCACAACTGCTCCTG | |
| | CCATTTGTCATGATGGAAAAGCACACTTTCCTCGTGAAGGTGTCTTTGTT | |
| | TCAAATGGCACACACTGGTTTGTAACACAAAGGAATTTTTATGAACCACA | |
| | AATCATTACTACAGACAACACATTTGTGTCTGGTAACTGTGATGTTGTAA | |
| | TAGGAATTGTCAACAACACAGTTTATGATCCTTTGCAACCTGAATTAGAC | |
| | TCATTCAAGGAGGAGTTAGATAAATATTTTAAGAATCATACATCACCAGA | |
| | TGTTGATTTAGGTGACATCTCTGGCATTAATGCTTCAGTTGTAAACATTC | |
| | AAAAAGAAATTGACCGCCTCAATGAGGTTGCCAAGAATTTAAATGAATCT | |
| | CTCATCGATCTCCAAGAACTTGGAAAGTATGAGCAGTATATAAAATGGCC | |
| | ATGGTACATTTGGCTAGGTTTTATAGCTGGCTTGATTGCCATAGTAATGG | |
| | TGACAATTATGCTTTGCTGTATGACCAGTTGCTGTAGTTGTCTCAAGGGC | |
| | TGTTGTTCTTGTGGATCCTGCTGCAAATTTGATGAAGACGACTCTGAGCC | |
| | AGTGCTCAAAGGAGTCAAATTAGATTACACATAAACGAACTTATGGATTT | |
| | GTTTATGAGAATCTTCACAATTGGAACTGTAACTTTGAAGCAAGGTGAAA | |
| | TCAAGGATGCTACTCCTTCAGATTTTGTTCGCGCTACTGCAACGATACCG | |
| | ATACAAGCCTCACTCCCTTTCGGATGGCTTATTGTTGGCGTTGCACTTCT | |
| | TGCTGTTTTTCAGAGCGCTTCCAAAATCATAACCCTCAAAAAGAGATGGC | |
| | AACTAGCACTCTCCAAGGGTGTTCACTTTGTTTGCAACTTGCTGTTGTTG | |
| | TTTGTAACAGTTTACTCACACCTTTTGCTCGTTGCTGCTGGCCTTGAAGC | |
| | CCCTTTTCTCTATCTTTATGCTTTAGTCTACTTCTTGCAGAGTATAAACT | |
| | TTGTAAGAATAATAATGAGGCTTTGGCTTTGCTGGAAATGCCGTTCCAAA | |
| | AACCCATTACTTTATGATGCCAACTATTTTCTTTGCTGGCATACTAATTG | |
| | TTACGACTATTGTATACCTTACAATAGTGTAACTTCTTCAATTGTCATTA | |
| | CTTCAGGTGATGGCACAACAAGTCCTATTTCTGAACATGACTACCAGATT | |
| | GGTGGTTATACTGAAAAATGGGAATCTGGAGTAAAAGACTGTGTTGTATT | |
| | ACACAGTTACTTCACTTCAGACTATTACCAGCTGTACTCAACTCAATTGA | |
| | GTACAGACACTGGTGTTGAACATGTTACCTTCTTCATCTACAATAAAATT | |
| | GTTGATGAGCCTGAAGAACATGTCCAAATTCACACAATCGACGGTTCATC | |
| | CGGAGTTGTTAATCCAGTAATGGAACCAATTTATGATGAACCGACGACGA | |
| | CTACTAGCGTGCCTTTGTAAGCACAAGCTGATGAGTACGAACTTATGTAC | |
| | TCATTCGTTTCGGAAGAGACAGGTACGTTAATAGTTAATAGCGTACTTCT | |
| | TTTTCTTGCTTTCGTGGTATTCTTGCTAGTTACACTAGCCATCCTTACTG | |
| | CGCTTCGATTGTGTGCGTACTGCTGCAATATTGTTAACGTGAGTCTTGTA | |
| | AAACCTTCTTTTTACGTTTACTCTCGTGTTAAAAATCTGAATTCTTCTAG | |
| | AGTTCCTGATCTTCTGGTCTAAACGAACTAAATATTATATTAGTTTTTCT | |
| | GTTTGGAACTTTAATTTTAGCCATGGCAGATTCCAACGGTACTATTACCG | |
| | TTGAAGAGCTTAAAAAGCTCCTTGAACAATGGAACCTAGTAATAGGTTTC | |
| | CTATTCCTTACATGGATTTGTCTTCTACAATTTGCCTATGCCAACAGGAA | |
| | TAGGTTTTTGTATATAATTAAGTTAATTTTCCTCTGGCTGTTATGGCCAG | |
| | TAACTTTAGCTTGTTTTGTGCTTGCTGCTGTTTACAGAATAAATTGGATC | |
| | ACCGGTGGAATTGCTATCGCAATGGCTTGTCTTGTAGGCTTGATGTGGCT | |
| | CAGCTACTTCATTGCTTCTTTCAGACTGTTTGCGCGTACGCGTTCCATGT | |
| | GGTCATTCAATCCAGAAACTAACATTCTTCTCAACGTGCCACTCCATGGC | |
| | ACTATTCTGACCAGACCGCTTCTAGAAAGTGAACTCGTAATCGGAGCTGT | |
| | GATCCTTCGTGGACATCTTCGTATTGCTGGACACCATCTAGGACGCTGTG | |
| | ACATCAAGGACCTGCCTAAAGAAATCACTGTTGCTACATCACGAACGCTT | |
| | TCTTATTACAAATTGGGAGCTTCGCAGCGTGTAGCAGGTGACTCAGGTTT | |
| | TGCTGCATACAGTCGCTACAGGATTGGCAACTATAAATTAAACACAGACC | |
| | ATTCCAGTAGCAGTGACAATATTGCTTTGCTTGTACAGTAAGTGACAACA | |
| | GATGTTTCATCTCGTTGACTTTCAGGTTACTATAGCAGAGATATTACTAA | |
| | TTATTATGAGGACTTTTAAAGTTTCCATTTGGAATCTTGATTACATCATA | |
| | AACCTCATAATTAAAAATTTATCTAAGTCACTAACTGAGAATAAATATTC | |
| | TCAATTAGATGAAGAGCAACCAATGGAGATTGATTAAACGAACATGAAAA | |
| | TTATTCTTTTCTTGGCACTGATAACACTCGCTACTTGTGAGCTTTATCAC | |
| | TACCAAGAGTGTGTTAGAGGTACAACAGTACTTTTAAAAGAACCTTGCTC | |
| | TTCTGGAACATACGAGGGCAATTCACCATTTCATCCTCTAGCTGATAACA | |
| | AATTTGCACTGACTTGCTTTAGCACTCAATTTGCTTTTGCTTGTCCTGAC | |
| | GGCGTAAAACACGTCTATCAGTTACGTGCCAGATCAGTTTCACCTAAACT | |
| | GTTCATCAGACAAGAGGAAGTTCAAGAACTTTACTCTCCAATTTTTCTTA | |
| | TTGTTGCGGCAATAGTGTTTATAACACTTTGCTTCACACTCAAAAGAAAG | |
| | ACAGAATGATTGAACTTTCATTAATTGACTTCTATTTGTGCTTTTTAGCC | |
| | TTTCTGCTATTCCTTGTTTTAATTATGCTTATTATCTTTTGGTTCTCACT | |
| | TGAACTGCAAGATCATAATGAAACTTGTCACGCCTAAACGAACATGAAAT | |
| | TTCTTGTTTTCTTAGGAATCATCACAACTGTAGCTGCATTTCACCAAGAA | |
| | TGTAGTTTACAGTCATGTACTCAACATCAACCATATGTAGTTGATGACCC | |
| | GTGTCCTATTCACTTCTATTCTAAATGGTATATTAGAGTAGGAGCTAGAA | |
| | AATCAGCACCTTTAATTGAATTGTGCGTGGATGAGGCTGGTTCTAAATCA | |
| | CCCATTCAGTACATCGATATCGGTAATTATACAGTTTCCTGTTTACCTTT | |
| | TACAATTAATTGCCAGGAACCTAAATTGGGTAGTCTTGTAGTGCGTTGTT | |
| | CGTTCTATGAAGACTTTTTAGAGTATCATGACGTTCGTGTTGTTTTAGAT | |
| | TTCATCTAAACGAACAAACTAAAATGTCTGATAATGGACCCCAAAATCAG | |
| | CGAAATGCACCCCGCATTACGTTTGGTGGACCCTCAGATTCAACTGGCAG | |
| | TAACCAGAATGGAGAACGCAGTGGGGCGCGATCAAAACAACGTCGGCCCC | |
| | AAGGTTTACCCAATAATACTGCGTCTTGGTTCACCGCTCTCACTCAACAT | |
| | GGCAAGGAAGACCTTAAATTCCCTCGAGGACAAGGCGTTCCAATTAACAC | |
| | CAATAGCAGTCCAGATGACCAAATTGGCTACTACCGAAGAGCTACCAGAC | |
| | GAATTCGTGGTGGTGACGGTAAAATGAAAGATCTCAGTCCAAGATGGTAT | |
| | TTCTACTACCTAGGAACTGGGCCAGAAGCTGGACTTCCCTATGGTGCTAA | |
| | CAAAGACGGCATCATATGGGTTGCAACTGAGGGAGCCTTGAATACACCAA | |
| | AAGATCACATTGGCACCCGCAATCCTGCTAACAATGCTGCAATCGTGCTA | |
| | CAACTTCCTCAAGGAACAACATTGCCAAAAGGCTTCTACGCAGAAGGGAG | |
| | CAGAGGCGGCAGTCAAGCCTCTTCTCGTTCCTCATCACGTAGTCGCAACA | |
| | GTTCAAGAAATTCAACTCCAGGCAGCAGTAGGGGAACTTCTCCTGCTAGA | |
| | ATGGCTGGCAATGGCGGTGATGCTGCTCTTGCTTTGCTGCTGCTTGACAG | |
| | ATTGAACCAGCTTGAGAGCAAAATGTCTGGTAAAGGCCAACAACAACAAG | |
| | GCCAAACTGTCACTAAGAAATCTGCTGCTGAGGCTTCTAAGAAGCCTCGG | |
| | CAAAAACGTACTGCCACTAAAGCATACAATGTAACACAAGCTTTCGGCAG | |
| | ACGTGGTCCAGAACAAACCCAAGGAAATTTTGGGGACCAGGAACTAATCA | |
| | GACAAGGAACTGATTACAAACATTGGCCGCAAATTGCACAATTTGCCCCC | |
| | AGCGCTTCAGCGTTCTTCGGAATGTCGCGCATTGGCATGGAAGTCACACC | |
| | TTCGGGAACGTGGTTGACCTACACAGGTGCCATCAAATTGGATGACAAAG | |
| | ATCCAAATTTCAAAGATCAAGTCATTTTGCTGAATAAGCATATTGACGCA | |
| | TACAAAACATTCCCACCAACAGAGCCTAAAAAGGACAAAAAGAAGAAGGC | |
| | TGATGAAACTCAAGCCTTACCGCAGAGACAGAAGAAACAGCAAACTGTGA | |
| | CTCTTCTTCCTGCTGCAGATTTGGATGATTTCTCCAAACAATTGCAACAA | |
| | TCCATGAGCAGTGCTGACTCAACTCAGGCCTAAACTCATGCAGACCACAC | |
| | AAGGCAGATGGGCTATATAAACGTTTTCGCTTTTCCGTTTACGATATATA | |
| | GTCTACTCTTGTGCAGAATGAATTCTCGTAACTACATAGCACAAGTAGAT | |
| | GTAGTTAACTTTAATCTCACATAGCAATCTTTAATCAGTGTGTAACATTA | |
| | GGGAGGACTTGAAAGAGCCACCACATTTTCACCGAGGCCACGCGGAGTAC | |
| | GATCGAGTGTACAGTGAACAATGCTAGGGAGAGCTGCCTATATGGAAGAG | |
| | CCCTAATGTGTAAAATTAATTTTAGTAGTGCTATCCCCATGTGATTTTAA | |
| | TAGCTTCTTAGGAGAATGACAAAAAAAAAAAAAAAAAAAAAAAAAAAAAA | |
| | AAA | |
| SARS | ATATTAGGTTTTTACCTACCCAGGAAAAGCCAACCAACCTCGATCTCTTG | 78 |
| CoV_Refseq | TAGATCTGTTCTCTAAACGAACTTTAAAATCTGTGTAGCTGTCGCTCGGC | |
| | TGCATGCCTAGTGCACCTACGCAGTATAAACAATAATAAATTTTACTGTC | |
| | GTTGACAAGAAACGAGTAACTCGTCCCTCTTCTGCAGACTGCTTACGGTT | |
| | TCGTCCGTGTTGCAGTCGATCATCAGCATACCTAGGTTTCGTCCGGGTGT | |
| | GACCGAAAGGTAAGATGGAGAGCCTTGTTCTTGGTGTCAACGAGAAAACA | |
| | CACGTCCAACTCAGTTTGCCTGTCCTTCAGGTTAGAGACGTGCTAGTGCG | |
| | TGGCTTCGGGGACTCTGTGGAAGAGGCCCTATCGGAGGCACGTGAACACC | |
| | TCAAAAATGGCACTTGTGGTCTAGTAGAGCTGGAAAAAGGCGTACTGCCC | |
| | CAGCTTGAACAGCCCTATGTGTTCATTAAACGTTCTGATGCCTTAAGCAC | |
| | CAATCACGGCCACAAGGTCGTTGAGCTGGTTGCAGAAATGGACGGCATTC | |
| | AGTACGGTCGTAGCGGTATAACACTGGGAGTACTCGTGCCACATGTGGGC | |
| | GAAACCCCAATTGCATACCGCAATGTTCTTCTTCGTAAGAACGGTAATAA | |
| | GGGAGCCGGTGGTCATAGCTATGGCATCGATCTAAAGTCTTATGACTTAG | |
| | GTGACGAGCTTGGCACTGATCCCATTGAAGATTATGAACAAAACTGGAAC | |
| | ACTAAGCATGGCAGTGGTGCACTCCGTGAACTCACTCGTGAGCTCAATGG | |
| | AGGTGCAGTCACTCGCTATGTCGACAACAATTTCTGTGGCCCAGATGGGT | |
| | ACCCTCTTGATTGCATCAAAGATTTTCTCGCACGCGCGGGCAAGTCAATG | |
| | TGCACTCTTTCCGAACAACTTGATTACATCGAGTCGAAGAGAGGTGTCTA | |
| | CTGCTGCCGTGACCATGAGCATGAAATTGCCTGGTTCACTGAGCGCTCTG | |
| | ATAAGAGCTACGAGCACCAGACACCCTTCGAAATTAAGAGTGCCAAGAAA | |
| | TTTGACACTTTCAAAGGGGAATGCCCAAAGTTTGTGTTTCCTCTTAACTC | |
| | AAAAGTCAAAGTCATTCAACCACGTGTTGAAAAGAAAAAGACTGAGGGTT | |
| | TCATGGGGCGTATACGCTCTGTGTACCCTGTTGCATCTCCACAGGAGTGT | |
| | AACAATATGCACTTGTCTACCTTGATGAAATGTAATCATTGCGATGAAGT | |
| | TTCATGGCAGACGTGCGACTTTCTGAAAGCCACTTGTGAACATTGTGGCA | |
| | CTGAAAATTTAGTTATTGAAGGACCTACTACATGTGGGTACCTACCTACT | |
| | AATGCTGTAGTGAAAATGCCATGTCCTGCCTGTCAAGACCCAGAGATTGG | |
| | ACCTGAGCATAGTGTTGCAGATTATCACAACCACTCAAACATTGAAACTC | |
| | GACTCCGCAAGGGAGGTAGGACTAGATGTTTTGGAGGCTGTGTGTTTGCC | |
| | TATGTTGGCTGCTATAATAAGCGTGCCTACTGGGTTCCTCGTGCTAGTGC | |
| | TGATATTGGCTCAGGCCATACTGGCATTACTGGTGACAATGTGGAGACCT | |
| | TGAATGAGGATCTCCTTGAGATACTGAGTCGTGAACGTGTTAACATTAAC | |
| | ATTGTTGGCGATTTTCATTTGAATGAAGAGGTTGCCATCATTTTGGCATC | |
| | TTTCTCTGCTTCTACAAGTGCCTTTATTGACACTATAAAGAGTCTTGATT | |
| | ACAAGTCTTTCAAAACCATTGTTGAGTCCTGCGGTAACTATAAAGTTACC | |
| | AAGGGAAAGCCCGTAAAAGGTGCTTGGAACATTGGACAACAGAGATCAGT | |
| | TTTAACACCACTGTGTGGTTTTCCCTCACAGGCTGCTGGTGTTATCAGAT | |
| | CAATTTTTGCGCGCACACTTGATGCAGCAAACCACTCAATTCCTGATTTG | |
| | CAAAGAGCAGCTGTCACCATACTTGATGGTATTTCTGAACAGTCATTACG | |
| | TCTTGTCGACGCCATGGTTTATACTTCAGACCTGCTCACCAACAGTGTCA | |
| | TTATTATGGCATATGTAACTGGTGGTCTTGTACAACAGACTTCTCAGTGG | |
| | TTGTCTAATCTTTTGGGCACTACTGTTGAAAAACTCAGGCCTATCTTTGA | |
| | ATGGATTGAGGCGAAACTTAGTGCAGGAGTTGAATTTCTCAAGGATGCTT | |
| | GGGAGATTCTCAAATTTCTCATTACAGGTGTTTTTGACATCGTCAAGGGT | |
| | CAAATACAGGTTGCTTCAGATAACATCAAGGATTGTGTAAAATGCTTCAT | |
| | TGATGTTGTTAACAAGGCACTCGAAATGTGCATTGATCAAGTCACTATCG | |
| | CTGGCGCAAAGTTGCGATCACTCAACTTAGGTGAAGTCTTCATCGCTCAA | |
| | AGCAAGGGACTTTACCGTCAGTGTATACGTGGCAAGGAGCAGCTGCAACT | |
| | ACTCATGCCTCTTAAGGCACCAAAAGAAGTAACCTTTCTTGAAGGTGATT | |
| | CACATGACACAGTACTTACCTCTGAGGAGGTTGTTCTCAAGAACGGTGAA | |
| | CTCGAAGCACTCGAGACGCCCGTTGATAGCTTCACAAATGGAGCTATCGT | |
| | TGGCACACCAGTCTGTGTAAATGGCCTCATGCTCTTAGAGATTAAGGACA | |
| | AAGAACAATACTGCGCATTGTCTCCTGGTTTACTGGCTACAAACAATGTC | |
| | TTTCGCTTAAAAGGGGGTGCACCAATTAAAGGTGTAACCTTTGGAGAAGA | |
| | TACTGTTTGGGAAGTTCAAGGTTACAAGAATGTGAGAATCACATTTGAGC | |
| | TTGATGAACGTGTTGACAAAGTGCTTAATGAAAAGTGCTCTGTCTACACT | |
| | GTTGAATCCGGTACCGAAGTTACTGAGTTTGCATGTGTTGTAGCAGAGGC | |
| | TGTTGTGAAGACTTTACAACCAGTTTCTGATCTCCTTACCAACATGGGTA | |
| | TTGATCTTGATGAGTGGAGTGTAGCTACATTCTACTTATTTGATGATGCT | |
| | GGTGAAGAAAACTTTTCATCACGTATGTATTGTTCCTTTTACCCTCCAGA | |
| | TGAGGAAGAAGAGGACGATGCAGAGTGTGAGGAAGAAGAAATTGATGAAA | |
| | CCTGTGAACATGAGTACGGTACAGAGGATGATTATCAAGGTCTCCCTCTG | |
| | GAATTTGGTGCCTCAGCTGAAACAGTTCGAGTTGAGGAAGAAGAAGAGGA | |
| | AGACTGGCTGGATGATACTACTGAGCAATCAGAGATTGAGCCAGAACCAG | |
| | AACCTACACCTGAAGAACCAGTTAATCAGTTTACTGGTTATTTAAAACTT | |
| | ACTGACAATGTTGCCATTAAATGTGTTGACATCGTTAAGGAGGCACAAAG | |
| | TGCTAATCCTATGGTGATTGTAAATGCTGCTAACATACACCTGAAACATG | |
| | GTGGTGGTGTAGCAGGTGCACTCAACAAGGCAACCAATGGTGCCATGCAA | |
| | AAGGAGAGTGATGATTACATTAAGCTAAATGGCCCTCTTACAGTAGGAGG | |
| | GTCTTGTTTGCTTTCTGGACATAATCTTGCTAAGAAGTGTCTGCATGTTG | |
| | TTGGACCTAACCTAAATGCAGGTGAGGACATCCAGCTTCTTAAGGCAGCA | |
| | TATGAAAATTTCAATTCACAGGACATCTTACTTGCACCATTGTTGTCAGC | |
| | AGGCATATTTGGTGCTAAACCACTTCAGTCTTTACAAGTGTGCGTGCAGA | |
| | CGGTTCGTACACAGGTTTATATTGCAGTCAATGACAAAGCTCTTTATGAG | |
| | CAGGTTGTCATGGATTATCTTGATAACCTGAAGCCTAGAGTGGAAGCACC | |
| | TAAACAAGAGGAGCCACCAAACACAGAAGATTCCAAAACTGAGGAGAAAT | |
| | CTGTCGTACAGAAGCCTGTCGATGTGAAGCCAAAAATTAAGGCCTGCATT | |
| | GATGAGGTTACCACAACACTGGAAGAAACTAAGTTTCTTACCAATAAGTT | |
| | ACTCTTGTTTGCTGATATCAATGGTAAGCTTTACCATGATTCTCAGAACA | |
| | TGCTTAGAGGTGAAGATATGTCTTTCCTTGAGAAGGATGCACCTTACATG | |
| | GTAGGTGATGTTATCACTAGTGGTGATATCACTTGTGTTGTAATACCCTC | |
| | CAAAAAGGCTGGTGGCACTACTGAGATGCTCTCAAGAGCTTTGAAGAAAG | |
| | TGCCAGTTGATGAGTATATAACCACGTACCCTGGACAAGGATGTGCTGGT | |
| | TATACACTTGAGGAAGCTAAGACTGCTCTTAAGAAATGCAAATCTGCATT | |
| | TTATGTACTACCTTCAGAAGCACCTAATGCTAAGGAAGAGATTCTAGGAA | |
| | CTGTATCCTGGAATTTGAGAGAAATGCTTGCTCATGCTGAAGAGACAAGA | |
| | AAATTAATGCCTATATGCATGGATGTTAGAGCCATAATGGCAACCATCCA | |
| | ACGTAAGTATAAAGGAATTAAAATTCAAGAGGGCATCGTTGACTATGGTG | |
| | TCCGATTCTTCTTTTATACTAGTAAAGAGCCTGTAGCTTCTATTATTACG | |
| | AAGCTGAACTCTCTAAATGAGCCGCTTGTCACAATGCCAATTGGTTATGT | |
| | GACACATGGTTTTAATCTTGAAGAGGCTGCGCGCTGTATGCGTTCTCTTA | |
| | AAGCTCCTGCCGTAGTGTCAGTATCATCACCAGATGCTGTTACTACATAT | |
| | AATGGATACCTCACTTCGTCATCAAAGACATCTGAGGAGCACTTTGTAGA | |
| | AACAGTTTCTTTGGCTGGCTCTTACAGAGATTGGTCCTATTCAGGACAGC | |
| | GTACAGAGTTAGGTGTTGAATTTCTTAAGCGTGGTGACAAAATTGTGTAC | |
| | CACACTCTGGAGAGCCCCGTCGAGTTTCATCTTGACGGTGAGGTTCTTTC | |
| | ACTTGACAAACTAAAGAGTCTCTTATCCCTGCGGGAGGTTAAGACTATAA | |
| | AAGTGTTCACAACTGTGGACAACACTAATCTCCACACACAGCTTGTGGAT | |
| | ATGTCTATGACATATGGACAGCAGTTTGGTCCAACATACTTGGATGGTGC | |
| | TGATGTTACAAAAATTAAACCTCATGTAAATCATGAGGGTAAGACTTTCT | |
| | TTGTACTACCTAGTGATGACACACTACGTAGTGAAGCTTTCGAGTACTAC | |
| | CATACTCTTGATGAGAGTTTTCTTGGTAGGTACATGTCTGCTTTAAACCA | |
| | CACAAAGAAATGGAAATTTCCTCAAGTTGGTGGTTTAACTTCAATTAAAT | |
| | GGGCTGATAACAATTGTTATTTGTCTAGTGTTTTATTAGCACTTCAACAG | |
| | CTTGAAGTCAAATTCAATGCACCAGCACTTCAAGAGGCTTATTATAGAGC | |
| | CCGTGCTGGTGATGCTGCTAACTTTTGTGCACTCATACTCGCTTACAGTA | |
| | ATAAAACTGTTGGCGAGCTTGGTGATGTCAGAGAAACTATGACCCATCTT | |
| | CTACAGCATGCTAATTTGGAATCTGCAAAGCGAGTTCTTAATGTGGTGTG | |
| | TAAACATTGTGGTCAGAAAACTACTACCTTAACGGGTGTAGAAGCTGTGA | |
| | TGTATATGGGTACTCTATCTTATGATAATCTTAAGACAGGTGTTTCCATT | |
| | CCATGTGTGTGTGGTCGTGATGCTACACAATATCTAGTACAACAAGAGTC | |
| | TTCTTTTGTTATGATGTCTGCACCACCTGCTGAGTATAAATTACAGCAAG | |
| | GTACATTCTTATGTGCGAATGAGTACACTGGTAACTATCAGTGTGGTCAT | |
| | TACACTCATATAACTGCTAAGGAGACCCTCTATCGTATTGACGGAGCTCA | |
| | CCTTACAAAGATGTCAGAGTACAAAGGACCAGTGACTGATGTTTTCTACA | |
| | AGGAAACATCTTACACTACAACCATCAAGCCTGTGTCGTATAAACTCGAT | |
| | GGAGTTACTTACACAGAGATTGAACCAAAATTGGATGGGTATTATAAAAA | |
| | GGATAATGCTTACTATACAGAGCAGCCTATAGACCTTGTACCAACTCAAC | |
| | CATTACCAAATGCGAGTTTTGATAATTTCAAACTCACATGTTCTAACACA | |
| | AAATTTGCTGATGATTTAAATCAAATGACAGGCTTCACAAAGCCAGCTTC | |
| | ACGAGAGCTATCTGTCACATTCTTCCCAGACTTGAATGGCGATGTAGTGG | |
| | CTATTGACTATAGACACTATTCAGCGAGTTTCAAGAAAGGTGCTAAATTA | |
| | CTGCATAAGCCAATTGTTTGGCACATTAACCAGGCTACAACCAAGACAAC | |
| | GTTCAAACCAAACACTTGGTGTTTACGTTGTCTTTGGAGTACAAAGCCAG | |
| | TAGATACTTCAAATTCATTTGAAGTTCTGGCAGTAGAAGACACACAAGGA | |
| | ATGGACAATCTTGCTTGTGAAAGTCAACAACCCACCTCTGAAGAAGTAGT | |
| | GGAAAATCCTACCATACAGAAGGAAGTCATAGAGTGTGACGTGAAAACTA | |
| | CCGAAGTTGTAGGCAATGTCATACTTAAACCATCAGATGAAGGTGTTAAA | |
| | GTAACACAAGAGTTAGGTCATGAGGATCTTATGGCTGCTTATGTGGAAAA | |
| | CACAAGCATTACCATTAAGAAACCTAATGAGCTTTCACTAGCCTTAGGTT | |
| | TAAAAACAATTGCCACTCATGGTATTGCTGCAATTAATAGTGTTCCTTGG | |
| | AGTAAAATTTTGGCTTATGTCAAACCATTCTTAGGACAAGCAGCAATTAC | |
| | AACATCAAATTGCGCTAAGAGATTAGCACAACGTGTGTTTAACAATTATA | |
| | TGCCTTATGTGTTTACATTATTGTTCCAATTGTGTACTTTTACTAAAAGT | |
| | ACCAATTCTAGAATTAGAGCTTCACTACCTACAACTATTGCTAAAAATAG | |
| | TGTTAAGAGTGTTGCTAAATTATGTTTGGATGCCGGCATTAATTATGTGA | |
| | AGTCACCCAAATTTTCTAAATTGTTCACAATCGCTATGTGGCTATTGTTG | |
| | TTAAGTATTTGCTTAGGTTCTCTAATCTGTGTAACTGCTGCTTTTGGTGT | |
| | ACTCTTATCTAATTTTGGTGCTCCTTCTTATTGTAATGGCGTTAGAGAAT | |
| | TGTATCTTAATTCGTCTAACGTTACTACTATGGATTTCTGTGAAGGTTCT | |
| | TTTCCTTGCAGCATTTGTTTAAGTGGATTAGACTCCCTTGATTCTTATCC | |
| | AGCTCTTGAAACCATTCAGGTGACGATTTCATCGTACAAGCTAGACTTGA | |
| | CAATTTTAGGTCTGGCCGCTGAGTGGGTTTTGGCATATATGTTGTTCACA | |
| | AAATTCTTTTATTTATTAGGTCTTTCAGCTATAATGCAGGTGTTCTTTGG | |
| | CTATTTTGCTAGTCATTTCATCAGCAATTCTTGGCTCATGTGGTTTATCA | |
| | TTAGTATTGTACAAATGGCACCCGTTTCTGCAATGGTTAGGATGTACATC | |
| | TTCTTTGCTTCTTTCTACTACATATGGAAGAGCTATGTTCATATCATGGA | |
| | TGGTTGCACCTCTTCGACTTGCATGATGTGCTATAAGCGCAATCGTGCCA | |
| | CACGCGTTGAGTGTACAACTATTGTTAATGGCATGAAGAGATCTTTCTAT | |
| | GTCTATGCAAATGGAGGCCGTGGCTTCTGCAAGACTCACAATTGGAATTG | |
| | TCTCAATTGTGACACATTTTGCACTGGTAGTACATTCATTAGTGATGAAG | |
| | TTGCTCGTGATTTGTCACTCCAGTTTAAAAGACCAATCAACCCTACTGAC | |
| | CAGTCATCGTATATTGTTGATAGTGTTGCTGTGAAAAATGGCGCGCTTCA | |
| | CCTCTACTTTGACAAGGCTGGTCAAAAGACCTATGAGAGACATCCGCTCT | |
| | CCCATTTTGTCAATTTAGACAATTTGAGAGCTAACAACACTAAAGGTTCA | |
| | CTGCCTATTAATGTCATAGTTTTTGATGGCAAGTCCAAATGCGACGAGTC | |
| | TGCTTCTAAGTCTGCTTCTGTGTACTACAGTCAGCTGATGTGCCAACCTA | |
| | TTCTGTTGCTTGACCAAGCTCTTGTATCAGACGTTGGAGATAGTACTGAA | |
| | GTTTCCGTTAAGATGTTTGATGCTTATGTCGACACCTTTTCAGCAACTTT | |
| | TAGTGTTCCTATGGAAAAACTTAAGGCACTTGTTGCTACAGCTCACAGCG | |
| | AGTTAGCAAAGGGTGTAGCTTTAGATGGTGTCCTTTCTACATTCGTGTCA | |
| | GCTGCCCGACAAGGTGTTGTTGATACCGATGTTGACACAAAGGATGTTAT | |
| | TGAATGTCTCAAACTTTCACATCACTCTGACTTAGAAGTGACAGGTGACA | |
| | GTTGTAACAATTTCATGCTCACCTATAATAAGGTTGAAAACATGACGCCC | |
| | AGAGATCTTGGCGCATGTATTGACTGTAATGCAAGGCATATCAATGCCCA | |
| | AGTAGCAAAAAGTCACAATGTTTCACTCATCTGGAATGTAAAAGACTACA | |
| | TGTCTTTATCTGAACAGCTGCGTAAACAAATTCGTAGTGCTGCCAAGAAG | |
| | AACAACATACCTTTTAGACTAACTTGTGCTACAACTAGACAGGTTGTCAA | |
| | TGTCATAACTACTAAAATCTCACTCAAGGGTGGTAAGATTGTTAGTACTT | |
| | GTTTTAAACTTATGCTTAAGGCCACATTATTGTGCGTTCTTGCTGCATTG | |
| | GTTTGTTATATCGTTATGCCAGTACATACATTGTCAATCCATGATGGTTA | |
| | CACAAATGAAATCATTGGTTACAAAGCCATTCAGGATGGTGTCACTCGTG | |
| | ACATCATTTCTACTGATGATTGTTTTGCAAATAAACATGCTGGTTTTGAC | |
| | GCATGGTTTAGCCAGCGTGGTGGTTCATACAAAAATGACAAAAGCTGCCC | |
| | TGTAGTAGCTGCTATCATTACAAGAGAGATTGGTTTCATAGTGCCTGGCT | |
| | TACCGGGTACTGTGCTGAGAGCAATCAATGGTGACTTCTTGCATTTTCTA | |
| | CCTCGTGTTTTTAGTGCTGTTGGCAACATTTGCTACACACCTTCCAAACT | |
| | CATTGAGTATAGTGATTTTGCTACCTCTGCTTGCGTTCTTGCTGCTGAGT | |
| | GTACAATTTTTAAGGATGCTATGGGCAAACCTGTGCCATATTGTTATGAC | |
| | ACTAATTTGCTAGAGGGTTCTATTTCTTATAGTGAGCTTCGTCCAGACAC | |
| | TCGTTATGTGCTTATGGATGGTTCCATCATACAGTTTCCTAACACTTACC | |
| | TGGAGGGTTCTGTTAGAGTAGTAACAACTTTTGATGCTGAGTACTGTAGA | |
| | CATGGTACATGCGAAAGGTCAGAAGTAGGTATTTGCCTATCTACCAGTGG | |
| | TAGATGGGTTCTTAATAATGAGCATTACAGAGCTCTATCAGGAGTTTTCT | |
| | GTGGTGTTGATGCGATGAATCTCATAGCTAACATCTTTACTCCTCTTGTG | |
| | CAACCTGTGGGTGCTTTAGATGTGTCTGCTTCAGTAGTGGCTGGTGGTAT | |
| | TATTGCCATATTGGTGACTTGTGCTGCCTACTACTTTATGAAATTCAGAC | |
| | GTGTTTTTGGTGAGTACAACCATGTTGTTGCTGCTAATGCACTTTTGTTT | |
| | TTGATGTCTTTCACTATACTCTGTCTGGTACCAGCTTACAGCTTTCTGCC | |
| | GGGAGTCTACTCAGTCTTTTACTTGTACTTGACATTCTATTTCACCAATG | |
| | ATGTTTCATTCTTGGCTCACCTTCAATGGTTTGCCATGTTTTCTCCTATT | |
| | GTGCCTTTTTGGATAACAGCAATCTATGTATTCTGTATTTCTCTGAAGCA | |
| | CTGCCATTGGTTCTTTAACAACTATCTTAGGAAAAGAGTCATGTTTAATG | |
| | GAGTTACATTTAGTACCTTCGAGGAGGCTGCTTTGTGTACCTTTTTGCTC | |
| | AACAAGGAAATGTACCTAAAATTGCGTAGCGAGACACTGTTGCCACTTAC | |
| | ACAGTATAACAGGTATCTTGCTCTATATAACAAGTACAAGTATTTCAGTG | |
| | GAGCCTTAGATACTACCAGCTATCGTGAAGCAGCTTGCTGCCACTTAGCA | |
| | AAGGCTCTAAATGACTTTAGCAACTCAGGTGCTGATGTTCTCTACCAACC | |
| | ACCACAGACATCAATCACTTCTGCTGTTCTGCAGAGTGGTTTTAGGAAAA | |
| | TGGCATTCCCGTCAGGCAAAGTTGAAGGGTGCATGGTACAAGTAACCTGT | |
| | GGAACTACAACTCTTAATGGATTGTGGTTGGATGACACAGTATACTGTCC | |
| | AAGACATGTCATTTGCACAGCAGAAGACATGCTTAATCCTAACTATGAAG | |
| | ATCTGCTCATTCGCAAATCCAACCATAGCTTTCTTGTTCAGGCTGGCAAT | |
| | GTTCAACTTCGTGTTATTGGCCATTCTATGCAAAATTGTCTGCTTAGGCT | |
| | TAAAGTTGATACTTCTAACCCTAAGACACCCAAGTATAAATTTGTCCGTA | |
| | TCCAACCTGGTCAAACATTTTCAGTTCTAGCATGCTACAATGGTTCACCA | |
| | TCTGGTGTTTATCAGTGTGCCATGAGACCTAATCATACCATTAAAGGTTC | |
| | TTTCCTTAATGGATCATGTGGTAGTGTTGGTTTTAACATTGATTATGATT | |
| | GCGTGTCTTTCTGCTATATGCATCATATGGAGCTTCCAACAGGAGTACAC | |
| | GCTGGTACTGACTTAGAAGGTAAATTCTATGGTCCATTTGTTGACAGACA | |
| | AACTGCACAGGCTGCAGGTACAGACACAACCATAACATTAAATGTTTTGG | |
| | CATGGCTGTATGCTGCTGTTATCAATGGTGATAGGTGGTTTCTTAATAGA | |
| | TTCACCACTACTTTGAATGACTTTAACCTTGTGGCAATGAAGTACAACTA | |
| | TGAACCTTTGACACAAGATCATGTTGACATATTGGGACCTCTTTCTGCTC | |
| | AAACAGGAATTGCCGTCTTAGATATGTGTGCTGCTTTGAAAGAGCTGCTG | |
| | CAGAATGGTATGAATGGTCGTACTATCCTTGGTAGCACTATTTTAGAAGA | |
| | TGAGTTTACACCATTTGATGTTGTTAGACAATGCTCTGGTGTTACCTTCC | |
| | AAGGTAAGTTCAAGAAAATTGTTAAGGGCACTCATCATTGGATGCTTTTA | |
| | ACTTTCTTGACATCACTATTGATTCTTGTTCAAAGTACACAGTGGTCACT | |
| | GTTTTTCTTTGTTTACGAGAATGCTTTCTTGCCATTTACTCTTGGTATTA | |
| | TGGCAATTGCTGCATGTGCTATGCTGCTTGTTAAGCATAAGCACGCATTC | |
| | TTGTGCTTGTTTCTGTTACCTTCTCTTGCAACAGTTGCTTACTTTAATAT | |
| | GGTCTACATGCCTGCTAGCTGGGTGATGCGTATCATGACATGGCTTGAAT | |
| | TGGCTGACACTAGCTTGTCTGGTTATAGGCTTAAGGATTGTGTTATGTAT | |
| | GCTTCAGCTTTAGTTTTGCTTATTCTCATGACAGCTCGCACTGTTTATGA | |
| | TGATGCTGCTAGACGTGTTTGGACACTGATGAATGTCATTACACTTGTTT | |
| | ACAAAGTCTACTATGGTAATGCTTTAGATCAAGCTATTTCCATGTGGGCC | |
| | TTAGTTATTTCTGTAACCTCTAACTATTCTGGTGTCGTTACGACTATCAT | |
| | GTTTTTAGCTAGAGCTATAGTGTTTGTGTGTGTTGAGTATTACCCATTGT | |
| | TATTTATTACTGGCAACACCTTACAGTGTATCATGCTTGTTTATTGTTTC | |
| | TTAGGCTATTGTTGCTGCTGCTACTTTGGCCTTTTCTGTTTACTCAACCG | |
| | TTACTTCAGGCTTACTCTTGGTGTTTATGACTACTTGGTCTCTACACAAG | |
| | AATTTAGGTATATGAACTCCCAGGGGCTTTTGCCTCCTAAGAGTAGTATT | |
| | GATGCTTTCAAGCTTAACATTAAGTTGTTGGGTATTGGAGGTAAACCATG | |
| | TATCAAGGTTGCTACTGTACAGTCTAAAATGTCTGACGTAAAGTGCACAT | |
| | CTGTGGTACTGCTCTCGGTTCTTCAACAACTTAGAGTAGAGTCATCTTCT | |
| | AAATTGTGGGCACAATGTGTACAACTCCACAATGATATTCTTCTTGCAAA | |
| | AGACACAACTGAAGCTTTCGAGAAGATGGTTTCTCTTTTGTCTGTTTTGC | |
| | TATCCATGCAGGGTGCTGTAGACATTAATAGGTTGTGCGAGGAAATGCTC | |
| | GATAACCGTGCTACTCTTCAGGCTATTGCTTCAGAATTTAGTTCTTTACC | |
| | ATCATATGCCGCTTATGCCACTGCCCAGGAGGCCTATGAGCAGGCTGTAG | |
| | CTAATGGTGATTCTGAAGTCGTTCTCAAAAAGTTAAAGAAATCTTTGAAT | |
| | GTGGCTAAATCTGAGTTTGACCGTGATGCTGCCATGCAACGCAAGTTGGA | |
| | AAAGATGGCAGATCAGGCTATGACCCAAATGTACAAACAGGCAAGATCTG | |
| | AGGACAAGAGGGCAAAAGTAACTAGTGCTATGCAAACAATGCTCTTCACT | |
| | ATGCTTAGGAAGCTTGATAATGATGCACTTAACAACATTATCAACAATGC | |
| | GCGTGATGGTTGTGTTCCACTCAACATCATACCATTGACTACAGCAGCCA | |
| | AACTCATGGTTGTTGTCCCTGATTATGGTACCTACAAGAACACTTGTGAT | |
| | GGTAACACCTTTACATATGCATCTGCACTCTGGGAAATCCAGCAAGTTGT | |
| | TGATGCGGATAGCAAGATTGTTCAACTTAGTGAAATTAACATGGACAATT | |
| | CACCAAATTTGGCTTGGCCTCTTATTGTTACAGCTCTAAGAGCCAACTCA | |
| | GCTGTTAAACTACAGAATAATGAACTGAGTCCAGTAGCACTACGACAGAT | |
| | GTCCTGTGCGGCTGGTACCACACAAACAGCTTGTACTGATGACAATGCAC | |
| | TTGCCTACTATAACAATTCGAAGGGAGGTAGGTTTGTGCTGGCATTACTA | |
| | TCAGACCACCAAGATCTCAAATGGGCTAGATTCCCTAAGAGTGATGGTAC | |
| | AGGTACAATTTACACAGAACTGGAACCACCTTGTAGGTTTGTTACAGACA | |
| | CACCAAAAGGGCCTAAAGTGAAATACTTGTACTTCATCAAAGGCTTAAAC | |
| | AACCTAAATAGAGGTATGGTGCTGGGCAGTTTAGCTGCTACAGTACGTCT | |
| | TCAGGCTGGAAATGCTACAGAAGTACCTGCCAATTCAACTGTGCTTTCCT | |
| | TCTGTGCTTTTGCAGTAGACCCTGCTAAAGCATATAAGGATTACCTAGCA | |
| | AGTGGAGGACAACCAATCACCAACTGTGTGAAGATGTTGTGTACACACAC | |
| | TGGTACAGGACAGGCAATTACTGTAACACCAGAAGCTAACATGGACCAAG | |
| | AGTCCTTTGGTGGTGCTTCATGTTGTCTGTATTGTAGATGCCACATTGAC | |
| | CATCCAAATCCTAAAGGATTCTGTGACTTGAAAGGTAAGTACGTCCAAAT | |
| | ACCTACCACTTGTGCTAATGACCCAGTGGGTTTTACACTTAGAAACACAG | |
| | TCTGTACCGTCTGCGGAATGTGGAAAGGTTATGGCTGTAGTTGTGACCAA | |
| | CTCCGCGAACCCTTGATGCAGTCTGCGGATGCATCAACGTTTTTAAACGG | |
| | GTTTGCGGTGTAAGTGCAGCCCGTCTTACACCGTGCGGCACAGGCACTAG | |
| | TACTGATGTCGTCTACAGGGCTTTTGATATTTACAACGAAAAAGTTGCTG | |
| | GTTTTGCAAAGTTCCTAAAAACTAATTGCTGTCGCTTCCAGGAGAAGGAT | |
| | GAGGAAGGCAATTTATTAGACTCTTACTTTGTAGTTAAGAGGCATACTAT | |
| | GTCTAACTACGAAGATGAAGAGACTATTTATAACTTGGTTAAAGATTGTC | |
| | CAGCGGTTGCTGTCCATGACTTTTTCAAGTTTAGAGTAGATGGTGACATG | |
| | GTACCACATATATCACGTCAGCGTCTAACTAAATACACAATGGCTGATTT | |
| | AGTCTATGCTCTACGTCATTTTGATGAGGGTAATTGTGATACATTAAAAG | |
| | AAATAGTCGTCACATACAATTGCTGTGATGATGATTATTTCAATAAGAAG | |
| | GATTGGTATGACTTCGTAGAGAATCCTGACATCTTACGCGTATATGCTAA | |
| | CTTAGGTGAGCGTGTACGCCAATCATTATTAAAGACTGTACAATTCTGCG | |
| | ATGCTATGCGTGATGCAGGCATTGTAGGCGTACTGACATTAGATAATCAG | |
| | GATCTTAATGGGAACTGGTACGATTTCGGTGATTTCGTACAAGTAGCACC | |
| | AGGCTGCGGAGTTCCTATTGTGGATTCATATTACTCATTGCTGATGCCCA | |
| | TCCTCACTTTGACTAGGGCATTGGCTGCTGAGTCCCATATGGATGCTGAT | |
| | CTCGCAAAACCACTTATTAAGTGGGATTTGCTGAAATATGATTTTACGGA | |
| | AGAGAGACTTTGTCTCTTCGACCGTTATTTTAAATATTGGGACCAGACAT | |
| | ACCATCCCAATTGTATTAACTGTTTGGATGATAGGTGTATCCTTCATTGT | |
| | GCAAACTTTAATGTGTTATTTTCTACTGTGTTTCCACCTACAAGTTTTGG | |
| | ACCACTAGTAAGAAAAATATTTGTAGATGGTGTTCCTTTTGTTGTTTCAA | |
| | CTGGATACCATTTTCGTGAGTTAGGAGTCGTACATAATCAGGATGTAAAC | |
| | TTACATAGCTCGCGTCTCAGTTTCAAGGAACTTTTAGTGTATGCTGCTGA | |
| | TCCAGCTATGCATGCAGCTTCTGGCAATTTATTGCTAGATAAACGCACTA | |
| | CATGCTTTTCAGTAGCTGCACTAACAAACAATGTTGCTTTTCAAACTGTC | |
| | AAACCCGGTAATTTTAATAAAGACTTTTATGACTTTGCTGTGTCTAAAGG | |
| | TTTCTTTAAGGAAGGAAGTTCTGTTGAACTAAAACACTTCTTCTTTGCTC | |
| | AGGATGGCAACGCTGCTATCAGTGATTATGACTATTATCGTTATAATCTG | |
| | CCAACAATGTGTGATATCAGACAACTCCTATTCGTAGTTGAAGTTGTTGA | |
| | TAAATACTTTGATTGTTACGATGGTGGCTGTATTAATGCCAACCAAGTAA | |
| | TCGTTAACAATCTGGATAAATCAGCTGGTTTCCCATTTAATAAATGGGGT | |
| | AAGGCTAGACTTTATTATGACTCAATGAGTTATGAGGATCAAGATGCACT | |
| | TTTCGCGTATACTAAGCGTAATGTCATCCCTACTATAACTCAAATGAATC | |
| | TTAAGTATGCCATTAGTGCAAAGAATAGAGCTCGCACCGTAGCTGGTGTC | |
| | TCTATCTGTAGTACTATGACAAATAGACAGTTTCATCAGAAATTATTGAA | |
| | GTCAATAGCCGCCACTAGAGGAGCTACTGTGGTAATTGGAACAAGCAAGT | |
| | TTTACGGTGGCTGGCATAATATGTTAAAAACTGTTTACAGTGATGTAGAA | |
| | ACTCCACACCTTATGGGTTGGGATTATCCAAAATGTGACAGAGCCATGCC | |
| | TAACATGCTTAGGATAATGGCCTCTCTTGTTCTTGCTCGCAAACATAACA | |
| | CTTGCTGTAACTTATCACACCGTTTCTACAGGTTAGCTAACGAGTGTGCG | |
| | CAAGTATTAAGTGAGATGGTCATGTGTGGCGGCTCACTATATGTTAAACC | |
| | AGGTGGAACATCATCCGGTGATGCTACAACTGCTTATGCTAATAGTGTCT | |
| | TTAACATTTGTCAAGCTGTTACAGCCAATGTAAATGCACTTCTTTCAACT | |
| | GATGGTAATAAGATAGCTGACAAGTATGTCCGCAATCTACAACACAGGCT | |
| | CTATGAGTGTCTCTATAGAAATAGGGATGTTGATCATGAATTCGTGGATG | |
| | AGTTTTACGCTTACCTGCGTAAACATTTCTCCATGATGATTCTTTCTGAT | |
| | GATGCCGTTGTGTGCTATAACAGTAACTATGCGGCTCAAGGTTTAGTAGC | |
| | TAGCATTAAGAACTTTAAGGCAGTTCTTTATTATCAAAATAATGTGTTCA | |
| | TGTCTGAGGCAAAATGTTGGACTGAGACTGACCTTACTAAAGGACCTCAC | |
| | GAATTTTGCTCACAGCATACAATGCTAGTTAAACAAGGAGATGATTACGT | |
| | GTACCTGCCTTACCCAGATCCATCAAGAATATTAGGCGCAGGCTGTTTTG | |
| | TCGATGATATTGTCAAAACAGATGGTACACTTATGATTGAAAGGTTCGTG | |
| | TCACTGGCTATTGATGCTTACCCACTTACAAAACATCCTAATCAGGAGTA | |
| | TGCTGATGTCTTTCACTTGTATTTACAATACATTAGAAAGTTACATGATG | |
| | AGCTTACTGGCCACATGTTGGACATGTATTCCGTAATGCTAACTAATGAT | |
| | AACACCTCACGGTACTGGGAACCTGAGTTTTATGAGGCTATGTACACACC | |
| | ACATACAGTCTTGCAGGCTGTAGGTGCTTGTGTATTGTGCAATTCACAGA | |
| | CTTCACTTCGTTGCGGTGCCTGTATTAGGAGACCATTCCTATGTTGCAAG | |
| | TGCTGCTATGACCATGTCATTTCAACATCACACAAATTAGTGTTGTCTGT | |
| | TAATCCCTATGTTTGCAATGCCCCAGGTTGTGATGTCACTGATGTGACAC | |
| | AACTGTATCTAGGAGGTATGAGCTATTATTGCAAGTCACATAAGCCTCCC | |
| | ATTAGTTTTCCATTATGTGCTAATGGTCAGGTTTTTGGTTTATACAAAAA | |
| | CACATGTGTAGGCAGTGACAATGTCACTGACTTCAATGCGATAGCAACAT | |
| | GTGATTGGACTAATGCTGGCGATTACATACTTGCCAACACTTGTACTGAG | |
| | AGACTCAAGCTTTTCGCAGCAGAAACGCTCAAAGCCACTGAGGAAACATT | |
| | TAAGCTGTCATATGGTATTGCCACTGTACGCGAAGTACTCTCTGACAGAG | |
| | AATTGCATCTTTCATGGGAGGTTGGAAAACCTAGACCACCATTGAACAGA | |
| | AACTATGTCTTTACTGGTTACCGTGTAACTAAAAATAGTAAAGTACAGAT | |
| | TGGAGAGTACACCTTTGAAAAAGGTGACTATGGTGATGCTGTTGTGTACA | |
| | GAGGTACTACGACATACAAGTTGAATGTTGGTGATTACTTTGTGTTGACA | |
| | TCTCACACTGTAATGCCACTTAGTGCACCTACTCTAGTGCCACAAGAGCA | |
| | CTATGTGAGAATTACTGGCTTGTACCCAACACTCAACATCTCAGATGAGT | |
| | TTTCTAGCAATGTTGCAAATTATCAAAAGGTCGGCATGCAAAAGTACTCT | |
| | ACACTCCAAGGACCACCTGGTACTGGTAAGAGTCATTTTGCCATCGGACT | |
| | TGCTCTCTATTACCCATCTGCTCGCATAGTGTATACGGCATGCTCTCATG | |
| | CAGCTGTTGATGCCCTATGTGAAAAGGCATTAAAATATTTGCCCATAGAT | |
| | AAATGTAGTAGAATCATACCTGCGCGTGCGCGCGTAGAGTGTTTTGATAA | |
| | ATTCAAAGTGAATTCAACACTAGAACAGTATGTTTTCTGCACTGTAAATG | |
| | CATTGCCAGAAACAACTGCTGACATTGTAGTCTTTGATGAAATCTCTATG | |
| | GCTACTAATTATGACTTGAGTGTTGTCAATGCTAGACTTCGTGCAAAACA | |
| | CTACGTCTATATTGGCGATCCTGCTCAATTACCAGCCCCCCGCACATTGC | |
| | TGACTAAAGGCACACTAGAACCAGAATATTTTAATTCAGTGTGCAGACTT | |
| | ATGAAAACAATAGGTCCAGACATGTTCCTTGGAACTTGTCGCCGTTGTCC | |
| | TGCTGAAATTGTTGACACTGTGAGTGCTTTAGTTTATGACAATAAGCTAA | |
| | AAGCACACAAGGATAAGTCAGCTCAATGCTTCAAAATGTTCTACAAAGGT | |
| | GTTATTACACATGATGTTTCATCTGCAATCAACAGACCTCAAATAGGCGT | |
| | TGTAAGAGAATTTCTTACACGCAATCCTGCTTGGAGAAAAGCTGTTTTTA | |
| | TCTCACCTTATAATTCACAGAACGCTGTAGCTTCAAAAATCTTAGGATTG | |
| | CCTACGCAGACTGTTGATTCATCACAGGGTTCTGAATATGACTATGTCAT | |
| | ATTCACACAAACTACTGAAACAGCACACTCTTGTAATGTCAACCGCTTCA | |
| | ATGTGGCTATCACAAGGGCAAAAATTGGCATTTTGTGCATAATGTCTGAT | |
| | AGAGATCTTTATGACAAACTGCAATTTACAAGTCTAGAAATACCACGTCG | |
| | CAATGTGGCTACATTACAAGCAGAAAATGTAACTGGACTTTTTAAGGACT | |
| | GTAGTAAGATCATTACTGGTCTTCATCCTACACAGGCACCTACACACCTC | |
| | AGCGTTGATATAAAGTTCAAGACTGAAGGATTATGTGTTGACATACCAGG | |
| | CATACCAAAGGACATGACCTACCGTAGACTCATCTCTATGATGGGTTTCA | |
| | AAATGAATTACCAAGTCAATGGTTACCCTAATATGTTTATCACCCGCGAA | |
| | GAAGCTATTCGTCACGTTCGTGCGTGGATTGGCTTTGATGTAGAGGGCTG | |
| | TCATGCAACTAGAGATGCTGTGGGTACTAACCTACCTCTCCAGCTAGGAT | |
| | TTTCTACAGGTGTTAACTTAGTAGCTGTACCGACTGGTTATGTTGACACT | |
| | GAAAATAACACAGAATTCACCAGAGTTAATGCAAAACCTCCACCAGGTGA | |
| | CCAGTTTAAACATCTTATACCACTCATGTATAAAGGCTTGCCCTGGAATG | |
| | TAGTGCGTATTAAGATAGTACAAATGCTCAGTGATACACTGAAAGGATTG | |
| | TCAGACAGAGTCGTGTTCGTCCTTTGGGCGCATGGCTTTGAGCTTACATC | |
| | AATGAAGTACTTTGTCAAGATTGGACCTGAAAGAACGTGTTGTCTGTGTG | |
| | ACAAACGTGCAACTTGCTTTTCTACTTCATCAGATACTTATGCCTGCTGG | |
| | AATCATTCTGTGGGTTTTGACTATGTCTATAACCCATTTATGATTGATGT | |
| | TCAGCAGTGGGGCTTTACGGGTAACCTTCAGAGTAACCATGACCAACATT | |
| | GCCAGGTACATGGAAATGCACATGTGGCTAGTTGTGATGCTATCATGACT | |
| | AGATGTTTAGCAGTCCATGAGTGCTTTGTTAAGCGCGTTGATTGGTCTGT | |
| | TGAATACCCTATTATAGGAGATGAACTGAGGGTTAATTCTGCTTGCAGAA | |
| | AAGTACAACACATGGTTGTGAAGTCTGCATTGCTTGCTGATAAGTTTCCA | |
| | GTTCTTCATGACATTGGAAATCCAAAGGCTATCAAGTGTGTGCCTCAGGC | |
| | TGAAGTAGAATGGAAGTTCTACGATGCTCAGCCATGTAGTGACAAAGCTT | |
| | ACAAAATAGAGGAACTCTTCTATTCTTATGCTACACATCACGATAAATTC | |
| | ACTGATGGTGTTTGTTTGTTTTGGAATTGTAACGTTGATCGTTACCCAGC | |
| | CAATGCAATTGTGTGTAGGTTTGACACAAGAGTCTTGTCAAACTTGAACT | |
| | TACCAGGCTGTGATGGTGGTAGTTTGTATGTGAATAAGCATGCATTCCAC | |
| | ACTCCAGCTTTCGATAAAAGTGCATTTACTAATTTAAAGCAATTGCCTTT | |
| | CTTTTACTATTCTGATAGTCCTTGTGAGTCTCATGGCAAACAAGTAGTGT | |
| | CGGATATTGATTATGTTCCACTCAAATCTGCTACGTGTATTACACGATGC | |
| | AATTTAGGTGGTGCTGTTTGCAGACACCATGCAAATGAGTACCGACAGTA | |
| | CTTGGATGCATATAATATGATGATTTCTGCTGGATTTAGCCTATGGATTT | |
| | ACAAACAATTTGATACTTATAACCTGTGGAATACATTTACCAGGTTACAG | |
| | AGTTTAGAAAATGTGGCTTATAATGTTGTTAATAAAGGACACTTTGATGG | |
| | ACACGCCGGCGAAGCACCTGTTTCCATCATTAATAATGCTGTTTACACAA | |
| | AGGTAGATGGTATTGATGTGGAGATCTTTGAAAATAAGACAACACTTCCT | |
| | GTTAATGTTGCATTTGAGCTTTGGGCTAAGCGTAACATTAAACCAGTGCC | |
| | AGAGATTAAGATACTCAATAATTTGGGTGTTGATATCGCTGCTAATACTG | |
| | TAATCTGGGACTACAAAAGAGAAGCCCCAGCACATGTATCTACAATAGGT | |
| | GTCTGCACAATGACTGACATTGCCAAGAAACCTACTGAGAGTGCTTGTTC | |
| | TTCACTTACTGTCTTGTTTGATGGTAGAGTGGAAGGACAGGTAGACCTTT | |
| | TTAGAAACGCCCGTAATGGTGTTTTAATAACAGAAGGTTCAGTCAAAGGT | |
| | CTAACACCTTCAAAGGGACCAGCACAAGCTAGCGTCAATGGAGTCACATT | |
| | AATTGGAGAATCAGTAAAAACACAGTTTAACTACTTTAAGAAAGTAGACG | |
| | GCATTATTCAACAGTTGCCTGAAACCTACTTTACTCAGAGCAGAGACTTA | |
| | GAGGATTTTAAGCCCAGATCACAAATGGAAACTGACTTTCTCGAGCTCGC | |
| | TATGGATGAATTCATACAGCGATATAAGCTCGAGGGCTATGCCTTCGAAC | |
| | ACATCGTTTATGGAGATTTCAGTCATGGACAACTTGGCGGTCTTCATTTA | |
| | ATGATAGGCTTAGCCAAGCGCTCACAAGATTCACCACTTAAATTAGAGGA | |
| | TTTTATCCCTATGGACAGCACAGTGAAAAATTACTTCATAACAGATGCGC | |
| | AAACAGGTTCATCAAAATGTGTGTGTTCTGTGATTGATCTTTTACTTGAT | |
| | GACTTTGTCGAGATAATAAAGTCACAAGATTTGTCAGTGATTTCAAAAGT | |
| | GGTCAAGGTTACAATTGACTATGCTGAAATTTCATTCATGCTTTGGTGTA | |
| | AGGATGGACATGTTGAAACCTTCTACCCAAAACTACAAGCAAGTCAAGCG | |
| | TGGCAACCAGGTGTTGCGATGCCTAACTTGTACAAGATGCAAAGAATGCT | |
| | TCTTGAAAAGTGTGACCTTCAGAATTATGGTGAAAATGCTGTTATACCAA | |
| | AAGGAATAATGATGAATGTCGCAAAGTATACTCAACTGTGTCAATACTTA | |
| | AATACACTTACTTTAGCTGTACCCTACAACATGAGAGTTATTCACTTTGG | |
| | TGCTGGCTCTGATAAAGGAGTTGCACCAGGTACAGCTGTGCTCAGACAAT | |
| | GGTTGCCAACTGGCACACTACTTGTCGATTCAGATCTTAATGACTTCGTC | |
| | TCCGACGCAGATTCTACTTTAATTGGAGACTGTGCAACAGTACATACGGC | |
| | TAATAAATGGGACCTTATTATTAGCGATATGTATGACCCTAGGACCAAAC | |
| | ATGTGACAAAAGAGAATGACTCTAAAGAAGGGTTTTTCACTTATCTGTGT | |
| | GGATTTATAAAGCAAAAACTAGCCCTGGGTGGTTCTATAGCTGTAAAGAT | |
| | AACAGAGCATTCTTGGAATGCTGACCTTTACAAGCTTATGGGCCATTTCT | |
| | CATGGTGGACAGCTTTTGTTACAAATGTAAATGCATCATCATCGGAAGCA | |
| | TTTTTAATTGGGGCTAACTATCTTGGCAAGCCGAAGGAACAAATTGATGG | |
| | CTATACCATGCATGCTAACTACATTTTCTGGAGGAACACAAATCCTATCC | |
| | AGTTGTCTTCCTATTCACTCTTTGACATGAGCAAATTTCCTCTTAAATTA | |
| | AGAGGAACTGCTGTAATGTCTCTTAAGGAGAATCAAATCAATGATATGAT | |
| | TTATTCTCTTCTGGAAAAAGGTAGGCTTATCATTAGAGAAAACAACAGAG | |
| | TTGTGGTTTCAAGTGATATTCTTGTTAACAACTAAACGAACATGTTTATT | |
| | TTCTTATTATTTCTTACTCTCACTAGTGGTAGTGACCTTGACCGGTGCAC | |
| | CACTTTTGATGATGTTCAAGCTCCTAATTACACTCAACATACTTCATCTA | |
| | TGAGGGGGGTTTACTATCCTGATGAAATTTTTAGATCAGACACTCTTTAT | |
| | TTAACTCAGGATTTATTTCTTCCATTTTATTCTAATGTTACAGGGTTTCA | |
| | TACTATTAATCATACGTTTGGCAACCCTGTCATACCTTTTAAGGATGGTA | |
| | TTTATTTTGCTGCCACAGAGAAATCAAATGTTGTCCGTGGTTGGGTTTTT | |
| | GGTTCTACCATGAACAACAAGTCACAGTCGGTGATTATTATTAACAATTC | |
| | TACTAATGTTGTTATACGAGCATGTAACTTTGAATTGTGTGACAACCCTT | |
| | TCTTTGCTGTTTCTAAACCCATGGGTACACAGACACATACTATGATATTC | |
| | GATAATGCATTTAATTGCACTTTCGAGTACATATCTGATGCCTTTTCGCT | |
| | TGATGTTTCAGAAAAGTCAGGTAATTTTAAACACTTACGAGAGTTTGTGT | |
| | TTAAAAATAAAGATGGGTTTCTCTATGTTTATAAGGGCTATCAACCTATA | |
| | GATGTAGTTCGTGATCTACCTTCTGGTTTTAACACTTTGAAACCTATTTT | |
| | TAAGTTGCCTCTTGGTATTAACATTACAAATTTTAGAGCCATTCTTACAG | |
| | CCTTTTCACCTGCTCAAGACATTTGGGGCACGTCAGCTGCAGCCTATTTT | |
| | GTTGGCTATTTAAAGCCAACTACATTTATGCTCAAGTATGATGAAAATGG | |
| | TACAATCACAGATGCTGTTGATTGTTCTCAAAATCCACTTGCTGAACTCA | |
| | AATGCTCTGTTAAGAGCTTTGAGATTGACAAAGGAATTTACCAGACCTCT | |
| | AATTTCAGGGTTGTTCCCTCAGGAGATGTTGTGAGATTCCCTAATATTAC | |
| | AAACTTGTGTCCTTTTGGAGAGGTTTTTAATGCTACTAAATTCCCTTCTG | |
| | TCTATGCATGGGAGAGAAAAAAAATTTCTAATTGTGTTGCTGATTACTCT | |
| | GTGCTCTACAACTCAACATTTTTTTCAACCTTTAAGTGCTATGGCGTTTC | |
| | TGCCACTAAGTTGAATGATCTTTGCTTCTCCAATGTCTATGCAGATTCTT | |
| | TTGTAGTCAAGGGAGATGATGTAAGACAAATAGCGCCAGGACAAACTGGT | |
| | GTTATTGCTGATTATAATTATAAATTGCCAGATGATTTCATGGGTTGTGT | |
| | CCTTGCTTGGAATACTAGGAACATTGATGCTACTTCAACTGGTAATTATA | |
| | ATTATAAATATAGGTATCTTAGACATGGCAAGCTTAGGCCCTTTGAGAGA | |
| | GACATATCTAATGTGCCTTTCTCCCCTGATGGCAAACCTTGCACCCCACC | |
| | TGCTCTTAATTGTTATTGGCCATTAAATGATTATGGTTTTTACACCACTA | |
| | CTGGCATTGGCTACCAACCTTACAGAGTTGTAGTACTTTCTTTTGAACTT | |
| | TTAAATGCACCGGCCACGGTTTGTGGACCAAAATTATCCACTGACCTTAT | |
| | TAAGAACCAGTGTGTCAATTTTAATTTTAATGGACTCACTGGTACTGGTG | |
| | TGTTAACTCCTTCTTCAAAGAGATTTCAACCATTTCAACAATTTGGCCGT | |
| | GATGTTTCTGATTTCACTGATTCCGTTCGAGATCCTAAAACATCTGAAAT | |
| | ATTAGACATTTCACCTTGCGCTTTTGGGGGTGTAAGTGTAATTACACCTG | |
| | GAACAAATGCTTCATCTGAAGTTGCTGTTCTATATCAAGATGTTAACTGC | |
| | ACTGATGTTTCTACAGCAATTCATGCAGATCAACTCACACCAGCTTGGCG | |
| | CATATATTCTACTGGAAACAATGTATTCCAGACTCAAGCAGGCTGTCTTA | |
| | TAGGAGCTGAGCATGTCGACACTTCTTATGAGTGCGACATTCCTATTGGA | |
| | GCTGGCATTTGTGCTAGTTACCATACAGTTTCTTTATTACGTAGTACTAG | |
| | CCAAAAATCTATTGTGGCTTATACTATGTCTTTAGGTGCTGATAGTTCAA | |
| | TTGCTTACTCTAATAACACCATTGCTATACCTACTAACTTTTCAATTAGC | |
| | ATTACTACAGAAGTAATGCCTGTTTCTATGGCTAAAACCTCCGTAGATTG | |
| | TAATATGTACATCTGCGGAGATTCTACTGAATGTGCTAATTTGCTTCTCC | |
| | AATATGGTAGCTTTTGCACACAACTAAATCGTGCACTCTCAGGTATTGCT | |
| | GCTGAACAGGATCGCAACACACGTGAAGTGTTCGCTCAAGTCAAACAAAT | |
| | GTACAAAACCCCAACTTTGAAATATTTTGGTGGTTTTAATTTTTCACAAA | |
| | TATTACCTGACCCTCTAAAGCCAACTAAGAGGTCTTTTATTGAGGACTTG | |
| | CTCTTTAATAAGGTGACACTCGCTGATGCTGGCTTCATGAAGCAATATGG | |
| | CGAATGCCTAGGTGATATTAATGCTAGAGATCTCATTTGTGCGCAGAAGT | |
| | TCAATGGACTTACAGTGTTGCCACCTCTGCTCACTGATGATATGATTGCT | |
| | GCCTACACTGCTGCTCTAGTTAGTGGTACTGCCACTGCTGGATGGACATT | |
| | TGGTGCTGGCGCTGCTCTTCAAATACCTTTTGCTATGCAAATGGCATATA | |
| | GGTTCAATGGCATTGGAGTTACCCAAAATGTTCTCTATGAGAACCAAAAA | |
| | CAAATCGCCAACCAATTTAACAAGGCGATTAGTCAAATTCAAGAATCACT | |
| | TACAACAACATCAACTGCATTGGGCAAGCTGCAAGACGTTGTTAACCAGA | |
| | ATGCTCAAGCATTAAACACACTTGTTAAACAACTTAGCTCTAATTTTGGT | |
| | GCAATTTCAAGTGTGCTAAATGATATCCTTTCGCGACTTGATAAAGTCGA | |
| | GGCGGAGGTACAAATTGACAGGTTAATTACAGGCAGACTTCAAAGCCTTC | |
| | AAACCTATGTAACACAACAACTAATCAGGGCTGCTGAAATCAGGGCTTCT | |
| | GCTAATCTTGCTGCTACTAAAATGTCTGAGTGTGTTCTTGGACAATCAAA | |
| | AAGAGTTGACTTTTGTGGAAAGGGCTACCACCTTATGTCCTTCCCACAAG | |
| | CAGCCCCGCATGGTGTTGTCTTCCTACATGTCACGTATGTGCCATCCCAG | |
| | GAGAGGAACTTCACCACAGCGCCAGCAATTTGTCATGAAGGCAAAGCATA | |
| | CTTCCCTCGTGAAGGTGTTTTTGTGTTTAATGGCACTTCTTGGTTTATTA | |
| | CACAGAGGAACTTCTTTTCTCCACAAATAATTACTACAGACAATACATTT | |
| | GTCTCAGGAAATTGTGATGTCGTTATTGGCATCATTAACAACACAGTTTA | |
| | TGATCCTCTGCAACCTGAGCTTGACTCATTCAAAGAAGAGCTGGACAAGT | |
| | ACTTCAAAAATCATACATCACCAGATGTTGATCTTGGCGACATTTCAGGC | |
| | ATTAACGCTTCTGTCGTCAACATTCAAAAAGAAATTGACCGCCTCAATGA | |
| | GGTCGCTAAAAATTTAAATGAATCACTCATTGACCTTCAAGAATTGGGAA | |
| | AATATGAGCAATATATTAAATGGCCTTGGTATGTTTGGCTCGGCTTCATT | |
| | GCTGGACTAATTGCCATCGTCATGGTTACAATCTTGCTTTGTTGCATGAC | |
| | TAGTTGTTGCAGTTGCCTCAAGGGTGCATGCTCTTGTGGTTCTTGCTGCA | |
| | AGTTTGATGAGGATGACTCTGAGCCAGTTCTCAAGGGTGTCAAATTACAT | |
| | TACACATAAACGAACTTATGGATTTGTTTATGAGATTTTTTACTCTTAGA | |
| | TCAATTACTGCACAGCCAGTAAAAATTGACAATGCTTCTCCTGCAAGTAC | |
| | TGTTCATGCTACAGCAACGATACCGCTACAAGCCTCACTCCCTTTCGGAT | |
| | GGCTTGTTATTGGCGTTGCATTTCTTGCTGTTTTTCAGAGCGCTACCAAA | |
| | ATAATTGCGCTCAATAAAAGATGGCAGCTAGCCCTTTATAAGGGCTTCCA | |
| | GTTCATTTGCAATTTACTGCTGCTATTTGTTACCATCTATTCACATCTTT | |
| | TGCTTGTCGCTGCAGGTATGGAGGCGCAATTTTTGTACCTCTATGCCTTG | |
| | ATATATTTTCTACAATGCATCAACGCATGTAGAATTATTATGAGATGTTG | |
| | GCTTTGTTGGAAGTGCAAATCCAAGAACCCATTACTTTATGATGCCAACT | |
| | ACTTTGTTTGCTGGCACACACATAACTATGACTACTGTATACCATATAAC | |
| | AGTGTCACAGATACAATTGTCGTTACTGAAGGTGACGGCATTTCAACACC | |
| | AAAACTCAAAGAAGACTACCAAATTGGTGGTTATTCTGAGGATAGGCACT | |
| | CAGGTGTTAAAGACTATGTCGTTGTACATGGCTATTTCACCGAAGTTTAC | |
| | TACCAGCTTGAGTCTACACAAATTACTACAGACACTGGTATTGAAAATGC | |
| | TACATTCTTCATCTTTAACAAGCTTGTTAAAGACCCACCGAATGTGCAAA | |
| | TACACACAATCGACGGCTCTTCAGGAGTTGCTAATCCAGCAATGGATCCA | |
| | ATTTATGATGAGCCGACGACGACTACTAGCGTGCCTTTGTAAGCACAAGA | |
| | AAGTGAGTACGAACTTATGTACTCATTCGTTTCGGAAGAAACAGGTACGT | |
| | TAATAGTTAATAGCGTACTTCTTTTTCTTGCTTTCGTGGTATTCTTGCTA | |
| | GTCACACTAGCCATCCTTACTGCGCTTCGATTGTGTGCGTACTGCTGCAA | |
| | TATTGTTAACGTGAGTTTAGTAAAACCAACGGTTTACGTCTACTCGCGTG | |
| | TTAAAAATCTGAACTCTTCTGAAGGAGTTCCTGATCTTCTGGTCTAAACG | |
| | AACTAACTATTATTATTATTCTGTTTGGAACTTTAACATTGCTTATCATG | |
| | GCAGACAACGGTACTATTACCGTTGAGGAGCTTAAACAACTCCTGGAACA | |
| | ATGGAACCTAGTAATAGGTTTCCTATTCCTAGCCTGGATTATGTTACTAC | |
| | AATTTGCCTATTCTAATCGGAACAGGTTTTTGTACATAATAAAGCTTGTT | |
| | TTCCTCTGGCTCTTGTGGCCAGTAACACTTGCTTGTTTTGTGCTTGCTGC | |
| | TGTCTACAGAATTAATTGGGTGACTGGCGGGATTGCGATTGCAATGGCTT | |
| | GTATTGTAGGCTTGATGTGGCTTAGCTACTTCGTTGCTTCCTTCAGGCTG | |
| | TTTGCTCGTACCCGCTCAATGTGGTCATTCAACCCAGAAACAAACATTCT | |
| | TCTCAATGTGCCTCTCCGGGGGACAATTGTGACCAGACCGCTCATGGAAA | |
| | GTGAACTTGTCATTGGTGCTGTGATCATTCGTGGTCACTTGCGAATGGCC | |
| | GGACACTCCCTAGGGCGCTGTGACATTAAGGACCTGCCAAAAGAGATCAC | |
| | TGTGGCTACATCACGAACGCTTTCTTATTACAAATTAGGAGCGTCGCAGC | |
| | GTGTAGGCACTGATTCAGGTTTTGCTGCATACAACCGCTACCGTATTGGA | |
| | AACTATAAATTAAATACAGACCACGCCGGTAGCAACGACAATATTGCTTT | |
| | GCTAGTACAGTAAGTGACAACAGATGTTTCATCTTGTTGACTTCCAGGTT | |
| | ACAATAGCAGAGATATTGATTATCATTATGAGGACTTTCAGGATTGCTAT | |
| | TTGGAATCTTGACGTTATAATAAGTTCAATAGTGAGACAATTATTTAAGC | |
| | CTCTAACTAAGAAGAATTATTCGGAGTTAGATGATGAAGAACCTATGGAG | |
| | TTAGATTATCCATAAAACGAACATGAAAATTATTCTCTTCCTGACATTGA | |
| | TTGTATTTACATCTTGCGAGCTATATCACTATCAGGAGTGTGTTAGAGGT | |
| | ACGACTGTACTACTAAAAGAACCTTGCCCATCAGGAACATACGAGGGCAA | |
| | TTCACCATTTCACCCTCTTGCTGACAATAAATTTGCACTAACTTGCACTA | |
| | GCACACACTTTGCTTTTGCTTGTGCTGACGGTACTCGACATACCTATCAG | |
| | CTGCGTGCAAGATCAGTTTCACCAAAACTTTTCATCAGACAAGAGGAGGT | |
| | TCAACAAGAGCTCTACTCGCCACTTTTTCTCATTGTTGCTGCTCTAGTAT | |
| | TTTTAATACTTTGCTTCACCATTAAGAGAAAGACAGAATGAATGAGCTCA | |
| | CTTTAATTGACTTCTATTTGTGCTTTTTAGCCTTTCTGCTATTCCTTGTT | |
| | TTAATAATGCTTATTATATTTTGGTTTTCACTCGAAATCCAGGATCTAGA | |
| | AGAACCTTGTACCAAAGTCTAAACGAACATGAAACTTCTCATTGTTTTGA | |
| | CTTGTATTTCTCTATGCAGTTGCATATGCACTGTAGTACAGCGCTGTGCA | |
| | TCTAATAAACCTCATGTGCTTGAAGATCCTTGTAAGGTACAACACTAGGG | |
| | GTAATACTTATAGCACTGCTTGGCTTTGTGCTCTAGGAAAGGTTTTACCT | |
| | TTTCATAGATGGCACACTATGGTTCAAACATGCACACCTAATGTTACTAT | |
| | CAACTGTCAAGATCCAGCTGGTGGTGCGCTTATAGCTAGGTGTTGGTACC | |
| | TTCATGAAGGTCACCAAACTGCTGCATTTAGAGACGTACTTGTTGTTTTA | |
| | AATAAACGAACAAATTAAAATGTCTGATAATGGACCCCAATCAAACCAAC | |
| | GTAGTGCCCCCCGCATTACATTTGGTGGACCCACAGATTCAACTGACAAT | |
| | AACCAGAATGGAGGACGCAATGGGGCAAGGCCAAAACAGCGCCGACCCCA | |
| | AGGTTTACCCAATAATACTGCGTCTTGGTTCACAGCTCTCACTCAGCATG | |
| | GCAAGGAGGAACTTAGATTCCCTCGAGGCCAGGGCGTTCCAATCAACACC | |
| | AATAGTGGTCCAGATGACCAAATTGGCTACTACCGAAGAGCTACCCGACG | |
| | AGTTCGTGGTGGTGACGGCAAAATGAAAGAGCTCAGCCCCAGATGGTACT | |
| | TCTATTACCTAGGAACTGGCCCAGAAGCTTCACTTCCCTACGGCGCTAAC | |
| | AAAGAAGGCATCGTATGGGTTGCAACTGAGGGAGCCTTGAATACACCCAA | |
| | AGACCACATTGGCACCCGCAATCCTAATAACAATGCTGCCACCGTGCTAC | |
| | AACTTCCTCAAGGAACAACATTGCCAAAAGGCTTCTACGCAGAGGGAAGC | |
| | AGAGGCGGCAGTCAAGCCTCTTCTCGCTCCTCATCACGTAGTCGCGGTAA | |
| | TTCAAGAAATTCAACTCCTGGCAGCAGTAGGGGAAATTCTCCTGCTCGAA | |
| | TGGCTAGCGGAGGTGGTGAAACTGCCCTCGCGCTATTGCTGCTAGACAGA | |
| | TTGAACCAGCTTGAGAGCAAAGTTTCTGGTAAAGGCCAACAACAACAAGG | |
| | CCAAACTGTCACTAAGAAATCTGCTGCTGAGGCATCTAAAAAGCCTCGCC | |
| | AAAAACGTACTGCCACAAAACAGTACAACGTCACTCAAGCATTTGGGAGA | |
| | CGTGGTCCAGAACAAACCCAAGGAAATTTCGGGGACCAAGACCTAATCAG | |
| | ACAAGGAACTGATTACAAACATTGGCCGCAAATTGCACAATTTGCTCCAA | |
| | GTGCCTCTGCATTCTTTGGAATGTCACGCATTGGCATGGAAGTCACACCT | |
| | TCGGGAACATGGCTGACTTATCATGGAGCCATTAAATTGGATGACAAAGA | |
| | TCCACAATTCAAAGACAACGTCATACTGCTGAACAAGCACATTGACGCAT | |
| | ACAAAACATTCCCACCAACAGAGCCTAAAAAGGACAAAAAGAAAAAGACT | |
| | GATGAAGCTCAGCCTTTGCCGCAGAGACAAAAGAAGCAGCCCACTGTGAC | |
| | TCTTCTTCCTGCGGCTGACATGGATGATTTCTCCAGACAACTTCAAAATT | |
| | CCATGAGTGGAGCTTCTGCTGATTCAACTCAGGCATAAACACTCATGATG | |
| | ACCACACAAGGCAGATGGGCTATGTAAACGTTTTCGCAATTCCGTTTACG | |
| | ATACATAGTCTACTCTTGTGCAGAATGAATTCTCGTAACTAAACAGCACA | |
| | AGTAGGTTTAGTTAACTTTAATCTCACATAGCAATCTTTAATCAATGTGT | |
| | AACATTAGGGAGGACTTGAAAGAGCCACCACATTTTCATCGAGGCCACGC | |
| | GGAGTACGATCGAGGGTACAGTGAATAATGCTAGGGAGAGCTGCCTATAT | |
| | GGAAGAGCCCTAATGTGTAAAATTAATTTTAGTAGTGCTATCCCCATGTG | |
| | ATTTTAATAGCTTCTTAGGAGAATGAGAAAAAAAAAAAAAAAAAAAAAAA | |
| | A | |
| MERS | GATTTAAGTGAATAGCTTGGCTATCTCACTTCCCCTCGTTCTCTTGCAGA | 79 |
| CoV_Refseq | ACTTTGATTTTAACGAACTTAAATAAAAGCCCTGTTGTTTAGCGTATCGT | |
| | TGCACTTGTCTGGTGGGATTGTGGCATTAATTTGCCTGCTCATCTAGGCA | |
| | GTGGACATATGCTCAACACTGGGTATAATTCTAATTGAATACTATTTTTC | |
| | AGTTAGAGCGTCGTGTCTCTTGTACGTCTCGGTCACAATACACGGTTTCG | |
| | TCCGGTGCGTGGCAATTCGGGGCACATCATGTCTTTCGTGGCTGGTGTGA | |
| | CCGCGCAAGGTGCGCGCGGTACGTATCGAGCAGCGCTCAACTCTGAAAAA | |
| | CATCAAGACCATGTGTCTCTAACTGTGCCACTCTGTGGTTCAGGAAACCT | |
| | GGTTGAAAAACTTTCACCATGGTTCATGGATGGCGAAAATGCCTATGAAG | |
| | TGGTGAAGGCCATGTTACTTAAAAAGGAGCCACTTCTCTATGTGCCCATC | |
| | CGGCTGGCTGGACACACTAGACACCTCCCAGGTCCTCGTGTGTACCTGGT | |
| | TGAGAGGCTCATTGCTTGTGAAAATCCATTCATGGTTAACCAATTGGCTT | |
| | ATAGCTCTAGTGCAAATGGCAGCCTGGTTGGCACAACTTTGCAGGGCAAG | |
| | CCTATTGGTATGTTCTTCCCTTATGACATCGAACTTGTCACAGGAAAGCA | |
| | AAATATTCTCCTGCGCAAGTATGGCCGTGGTGGTTATCACTACACCCCAT | |
| | TCCACTATGAGCGAGACAACACCTCTTGCCCTGAGTGGATGGACGATTTT | |
| | GAGGCGGATCCTAAAGGCAAATATGCCCAGAATCTGCTTAAGAAGTTGAT | |
| | TGGCGGTGATGTCACTCCAGTTGACCAATACATGTGTGGCGTTGATGGAA | |
| | AACCCATTAGTGCCTACGCATTTTTAATGGCCAAGGATGGAATAACCAAA | |
| | CTGGCTGATGTTGAAGCGGACGTCGCAGCACGTGCTGATGACGAAGGCTT | |
| | CATCACATTAAAGAACAATCTATATAGATTGGTTTGGCATGTTGAGCGTA | |
| | AAGACGTTCCATATCCTAAGCAATCTATTTTTACTATTAATAGTGTGGTC | |
| | CAAAAGGATGGTGTTGAAAACACTCCTCCTCACTATTTTACTCTTGGATG | |
| | CAAAATTTTAACGCTCACCCCACGCAACAAGTGGAGTGGCGTTTCTGACT | |
| | TGTCCCTCAAACAAAAACTCCTTTACACCTTCTATGGTAAGGAGTCACTT | |
| | GAGAACCCAACCTACATTTACCACTCCGCATTCATTGAGTGTGGAAGTTG | |
| | TGGTAATGATTCCTGGCTTACAGGGAATGCTATCCAAGGGTTTGCCTGTG | |
| | GATGTGGGGCATCATATACAGCTAATGATGTCGAAGTCCAATCATCTGGC | |
| | ATGATTAAGCCAAATGCTCTTCTTTGTGCTACTTGCCCCTTTGCTAAGGG | |
| | TGATAGCTGTTCTTCTAATTGCAAACATTCAGTTGCTCAGTTGGTTAGTT | |
| | ACCTTTCTGAACGCTGTAATGTTATTGCTGATTCTAAGTCCTTCACACTT | |
| | ATCTTTGGTGGCGTAGCTTACGCCTACTTTGGATGTGAGGAAGGTACTAT | |
| | GTACTTTGTGCCTAGAGCTAAGTCTGTTGTCTCAAGGATTGGAGACTCCA | |
| | TCTTTACAGGCTGTACTGGCTCTTGGAACAAGGTCACTCAAATTGCTAAC | |
| | ATGTTCTTGGAACAGACTCAGCATTCCCTTAACTTTGTGGGAGAGTTCGT | |
| | TGTCAACGATGTTGTCCTCGCAATTCTCTCTGGAACCACAACTAATGTTG | |
| | ACAAAATACGCCAGCTTCTCAAAGGTGTCACCCTTGACAAGTTGCGTGAT | |
| | TATTTAGCTGACTATGACGTAGCAGTCACTGCCGGCCCATTCATGGATAA | |
| | TGCTATTAATGTTGGTGGTACAGGATTACAGTATGCCGCCATTACTGCAC | |
| | CTTATGTAGTTCTCACTGGCTTAGGTGAGTCCTTTAAGAAAGTTGCAACC | |
| | ATACCGTATAAGGTTTGCAACTCTGTTAAGGATACTCTGGCTTATTATGC | |
| | TCACAGCGTGTTGTACAGAGTTTTTCCTTATGACATGGATTCTGGTGTGT | |
| | CATCCTTTAGTGAACTACTTTTTGATTGCGTTGATCTTTCAGTAGCTTCT | |
| | ACCTATTTTTTAGTCCGCATCTTGCAAGATAAGACTGGCGACTTTATGTC | |
| | TACAATTATTACTTCCTGCCAAACTGCTGTTAGTAAGCTTCTAGATACAT | |
| | GTTTTGAAGCTACAGAAGCAACATTTAACTTCTTGTTAGATTTGGCAGGA | |
| | TTGTTCAGAATCTTTCTCCGCAATGCCTATGTGTACACTTCACAAGGGTT | |
| | TGTGGTGGTCAATGGCAAAGTTTCTACACTTGTCAAACAAGTGTTAGACT | |
| | TGCTTAATAAGGGTATGCAACTTTTGCATACAAAGGTCTCCTGGGCTGGT | |
| | TCTAAAATCATTGCTGTTATCTACAGCGGCAGGGAGTCTCTAATATTCCC | |
| | ATCGGGAACCTATTACTGTGTCACCACTAAGGCTAAGTCCGTTCAACAAG | |
| | ATCTTGACGTTATTTTGCCTGGTGAGTTTTCCAAGAAGCAGTTAGGACTG | |
| | CTCCAACCTACTGACAATTCTACAACTGTTAGTGTTACTGTATCCAGTAA | |
| | CATGGTTGAAACTGTTGTGGGTCAACTTGAGCAAACTAATATGCATAGTC | |
| | CTGATGTTATAGTAGGTGACTATGTCATTATTAGTGAAAAATTGTTTGTG | |
| | CGTAGTAAGGAAGAAGACGGATTTGCCTTCTACCCTGCTTGCACTAATGG | |
| | TCATGCTGTACCGACTCTCTTTAGACTTAAGGGAGGTGCACCTGTAAAAA | |
| | AAGTAGCCTTTGGCGGTGATCAAGTACATGAGGTTGCTGCTGTAAGAAGT | |
| | GTTACTGTCGAGTACAACATTCATGCTGTATTAGACACACTACTTGCTTC | |
| | TTCTAGTCTTAGAACCTTTGTTGTAGATAAGTCTTTGTCAATTGAGGAGT | |
| | TTGCTGACGTAGTAAAGGAACAAGTCTCAGACTTGCTTGTTAAATTACTG | |
| | CGTGGAATGCCGATTCCAGATTTTGATTTAGACGATTTTATTGACGCACC | |
| | ATGCTATTGCTTTAACGCTGAGGGTGATGCATCCTGGTCTTCTACTATGA | |
| | TCTTCTCTCTTCACCCCGTCGAGTGTGACGAGGAGTGTTCTGAAGTAGAG | |
| | GCTTCAGATTTAGAAGAAGGTGAATCAGAGTGCATTTCTGAGACTTCAAC | |
| | TGAACAAGTTGACGTTTCTCATGAGACTTCTGACGACGAGTGGGCTGCTG | |
| | CAGTTGATGAAGCGTTCCCTCTCGATGAAGCAGAAGATGTTACTGAATCT | |
| | GTGCAAGAAGAAGCACAACCAGTAGAAGTACCTGTTGAAGATATTGCGCA | |
| | GGTTGTCATAGCTGACACCTTACAGGAAACTCCTGTTGTGCCTGATACTG | |
| | TTGAAGTCCCACCGCAAGTGGTGAAACTTCCGTCTGCACCTCAGACTATC | |
| | CAGCCCGAGGTAAAAGAAGTTGCACCTGTCTATGAGGCTGATACCGAACA | |
| | GACACAGAATGTTACTGTTAAACCTAAGAGGTTACGCAAAAAGCGTAATG | |
| | TTGACCCTTTGTCCAATTTTGAACATAAGGTTATTACAGAGTGCGTTACC | |
| | ATAGTTTTAGGTGACGCAATTCAAGTAGCCAAGTGCTATGGGGAGTCTGT | |
| | GTTAGTTAATGCTGCTAACACACATCTTAAGCATGGCGGTGGTATCGCTG | |
| | GTGCTATTAATGCGGCTTCAAAAGGGGCTGTCCAAAAAGAGTCAGATGAG | |
| | TATATTCTGGCTAAAGGGCCGTTACAAGTAGGAGATTCAGTTCTCTTGCA | |
| | AGGCCATTCTCTAGCTAAGAATATCCTGCATGTCGTAGGCCCAGATGCCC | |
| | GCGCTAAACAGGATGTTTCTCTCCTTAGTAAGTGCTATAAGGCTATGAAT | |
| | GCATATCCTCTTGTAGTCACTCCTCTTGTTTCAGCAGGCATATTTGGTGT | |
| | AAAACCAGCTGTGTCTTTTGATTATCTTATTAGGGAGGCTAAGACTAGAG | |
| | TTTTAGTCGTCGTTAATTCCCAAGATGTCTATAAGAGTCTTACCATAGTT | |
| | GACATTCCACAGAGTTTGACTTTTTCATATGATGGGTTACGTGGCGCAAT | |
| | ACGTAAAGCTAAAGATTATGGTTTTACTGTTTTTGTGTGCACAGACAACT | |
| | CTGCTAACACTAAAGTTCTTAGGAACAAGGGTGTTGATTATACTAAGAAG | |
| | TTTCTTACAGTTGACGGTGTGCAATATTATTGCTACACGTCTAAGGACAC | |
| | TTTAGATGATATCTTACAACAGGCTAATAAGTCTGTTGGTATTATATCTA | |
| | TGCCTTTGGGATATGTGTCTCATGGTTTAGACTTAATGCAAGCAGGGAGT | |
| | GTCGTGCGTAGAGTTAACGTGCCCTACGTGTGTCTCCTAGCTAATAAAGA | |
| | GCAAGAAGCTATTTTGATGTCTGAAGACGTTAAGTTAAACCCTTCAGAAG | |
| | ATTTTATAAAGCACGTCCGCACTAATGGTGGTTACAATTCTTGGCATTTA | |
| | GTCGAGGGTGAACTATTGGTGCAAGACTTACGCTTAAATAAGCTCCTGCA | |
| | TTGGTCTGATCAAACCATATGCTACAAGGATAGTGTGTTTTATGTTGTAA | |
| | AGAATAGTACAGCTTTTCCATTTGAAACACTTTCAGCATGTCGTGCGTAT | |
| | TTGGATTCACGCACGACACAGCAGTTAACAATCGAAGTCTTAGTGACTGT | |
| | CGATGGTGTAAATTTTAGAACAGTCGTTCTAAATAATAAGAACACTTATA | |
| | GATCACAGCTTGGATGCGTTTTCTTTAATGGTGCTGATATTTCTGACACC | |
| | ATTCCTGATGAGAAACAGAATGGTCACAGTTTATATCTAGCAGACAATTT | |
| | GACTGCTGATGAAACAAAGGCGCTTAAAGAGTTATATGGCCCCGTTGATC | |
| | CTACTTTCTTACACAGATTCTATTCACTTAAGGCTGCAGTCCATGGGTGG | |
| | AAGATGGTTGTGTGTGATAAGGTACGTTCTCTCAAATTGAGTGATAATAA | |
| | TTGTTATCTTAATGCAGTTATTATGACACTTGATTTATTGAAGGACATTA | |
| | AATTTGTTATACCTGCTCTACAGCATGCATTTATGAAACATAAGGGCGGT | |
| | GATTCAACTGACTTCATAGCCCTCATTATGGCTTATGGCAATTGCACATT | |
| | TGGTGCTCCAGATGATGCCTCTCGGTTACTTCATACCGTGCTTGCAAAGG | |
| | CTGAGTTATGCTGTTCTGCACGCATGGTTTGGAGAGAGTGGTGCAATGTC | |
| | TGTGGCATAAAAGATGTTGTTCTACAAGGCTTAAAAGCTTGTTGTTACGT | |
| | GGGTGTGCAAACTGTTGAAGATCTGCGTGCTCGCATGACATATGTATGCC | |
| | AGTGTGGTGGTGAACGTCATCGGCAATTAGTCGAACACACCACCCCCTGG | |
| | TTGCTGCTCTCAGGCACACCAAATGAAAAATTGGTGACAACCTCCACGGC | |
| | GCCTGATTTTGTAGCATTTAATGTCTTTCAGGGCATTGAAACGGCTGTTG | |
| | GCCATTATGTTCATGCTCGCCTGAAGGGTGGTCTTATTTTAAAGTTTGAC | |
| | TCTGGCACCGTTAGCAAGACTTCAGACTGGAAGTGCAAGGTGACAGATGT | |
| | ACTTTTCCCCGGCCAAAAATACAGTAGCGATTGTAATGTCGTACGGTATT | |
| | CTTTGGACGGTAATTTCAGAACAGAGGTTGATCCCGACCTATCTGCTTTC | |
| | TATGTTAAGGATGGTAAATACTTTACAAGTGAACCACCCGTAACATATTC | |
| | ACCAGCTACAATTTTAGCTGGTAGTGTCTACACTAATAGCTGCCTTGTAT | |
| | CGTCTGATGGACAACCTGGCGGTGATGCTATTAGTTTGAGTTTTAATAAC | |
| | CTTTTAGGGTTTGATTCTAGTAAACCAGTCACTAAGAAATACACTTACTC | |
| | CTTCTTGCCTAAAGAAGACGGCGATGTGTTGTTGGCTGAGTTTGACACTT | |
| | ATGACCCTATTTATAAGAATGGTGCCATGTATAAAGGCAAACCAATTCTT | |
| | TGGGTCAATAAAGCATCTTATGATACTAATCTTAATAAGTTCAATAGAGC | |
| | TAGTTTGCGTCAAATTTTTGACGTAGCCCCCATTGAACTCGAAAATAAAT | |
| | TCACACCTTTGAGTGTGGAGTCTACACCAGTTGAACCTCCAACTGTAGAT | |
| | GTGGTAGCACTTCAACAGGAAATGACAATTGTCAAATGTAAGGGTTTAAA | |
| | TAAACCTTTCGTGAAGGACAATGTCAGTTTCGTTGCTGATGATTCAGGTA | |
| | CTCCCGTTGTTGAGTATCTGTCTAAAGAAGACCTACATACATTGTATGTA | |
| | GACCCTAAGTATCAAGTCATTGTCTTAAAAGACAATGTACTTTCTTCTAT | |
| | GCTTAGATTGCACACCGTTGAGTCAGGTGATATTAACGTTGTTGCAGCTT | |
| | CCGGATCTTTGACACGTAAAGTGAAGTTACTATTTAGGGCTTCATTTTAT | |
| | TTCAAAGAATTTGCTACCCGCACTTTCACTGCTACCACTGCTGTAGGTAG | |
| | TTGTATAAAGAGTGTAGTGCGGCATCTAGGTGTTACTAAAGGCATATTGA | |
| | CAGGCTGTTTTAGTTTTGCCAAGATGTTATTTATGCTTCCACTAGCTTAC | |
| | TTTAGTGATTCAAAACTCGGCACCACAGAGGTTAAAGTGAGTGCTTTGAA | |
| | AACAGCCGGCGTTGTGACAGGTAATGTTGTAAAACAGTGTTGCACTGCTG | |
| | CTGTTGATTTAAGTATGGATAAGTTGCGCCGTGTGGATTGGAAATCAACC | |
| | CTACGGTTGTTACTTATGTTATGCACAACTATGGTATTGTTGTCTTCTGT | |
| | GTATCACTTGTATGTCTTCAATCAGGTCTTATCAAGTGATGTTATGTTTG | |
| | AAGATGCCCAAGGTTTGAAAAAGTTCTACAAAGAAGTTAGAGCTTACCTA | |
| | GGAATCTCTTCTGCTTGTGACGGTCTTGCTTCAGCTTATAGGGCGAATTC | |
| | CTTTGATGTACCTACATTCTGCGCAAACCGTTCTGCAATGTGTAATTGGT | |
| | GCTTGATTAGCCAAGATTCCATAACTCACTACCCAGCTCTTAAGATGGTT | |
| | CAAACACATCTTAGCCACTATGTTCTTAACATAGATTGGTTGTGGTTTGC | |
| | ATTTGAGACTGGTTTGGCATACATGCTCTATACCTCGGCCTTCAACTGGT | |
| | TGTTGTTGGCAGGTACATTGCATTATTTCTTTGCACAGACTTCCATATTT | |
| | GTAGACTGGCGGTCATACAATTATGCTGTGTCTAGTGCCTTCTGGTTATT | |
| | CACCCACATTCCAATGGCGGGTTTGGTACGAATGTATAATTTGTTAGCAT | |
| | GCCTTTGGCTTTTACGCAAGTTTTATCAGCATGTAATCAATGGTTGCAAA | |
| | GATACGGCATGCTTGCTCTGCTATAAGAGGAACCGACTTACTAGAGTTGA | |
| | AGCTTCTACCGTTGTCTGTGGTGGAAAACGTACGTTTTATATCACAGCAA | |
| | ATGGCGGTATTTCATTCTGTCGTAGGCATAATTGGAATTGTGTGGATTGT | |
| | GACACTGCAGGTGTGGGGAATACCTTCATCTGTGAAGAAGTCGCAAATGA | |
| | CCTCACTACCGCCCTACGCAGGCCTATTAACGCTACGGATAGATCACATT | |
| | ATTATGTGGATTCCGTTACAGTTAAAGAGACTGTTGTTCAGTTTAATTAT | |
| | CGTAGAGACGGTCAACCATTCTACGAGCGGTTTCCCCTCTGCGCTTTTAC | |
| | AAATCTAGATAAGTTGAAGTTCAAAGAGGTCTGTAAAACTACTACTGGTA | |
| | TACCTGAATACAACTTTATCATCTACGACTCATCAGATCGTGGCCAGGAA | |
| | AGTTTAGCTAGGTCTGCATGTGTTTATTATTCTCAAGTCTTGTGTAAATC | |
| | AATTCTTTTGGTTGACTCAAGTTTGGTTACTTCTGTTGGTGATTCTAGTG | |
| | AAATCGCCACTAAAATGTTTGATTCCTTTGTTAATAGTTTCGTCTCGCTG | |
| | TATAATGTCACACGCGATAAGTTGGAAAAACTTATCTCTACTGCTCGTGA | |
| | TGGCGTAAGGCGAGGCGATAACTTCCATAGTGTCTTAACAACATTCATTG | |
| | ACGCAGCACGAGGCCCCGCAGGTGTGGAGTCTGATGTTGAGACCAATGAA | |
| | ATTGTTGACTCTGTGCAGTATGCTCATAAACATGACATACAAATTACTAA | |
| | TGAGAGCTACAATAATTATGTACCCTCATATGTTAAACCTGATAGTGTGT | |
| | CTACCAGCGATTTAGGTAGTCTCATTGATTGTAATGCGGCTTCAGTTAAC | |
| | CAAATTGTCTTGCGTAATTCTAATGGTGCTTGCATTTGGAACGCTGCTGC | |
| | ATATATGAAACTCTCGGATGCACTTAAACGACAGATTCGCATTGCATGCC | |
| | GTAAGTGTAATTTAGCTTTCCGGTTAACCACCTCAAAGCTACGCGCTAAT | |
| | GATAATATCTTATCAGTTAGATTCACTGCTAACAAAATTGTTGGTGGTGC | |
| | TCCTACATGGTTTAATGCGTTGCGTGACTTTACGTTAAAGGGTTATGTTC | |
| | TTGCTACCATTATTGTGTTTCTGTGTGCTGTACTGATGTATTTGTGTTTA | |
| | CCTACATTTTCTATGGCACCTGTTGAATTTTATGAAGACCGCATCTTGGA | |
| | CTTTAAAGTTCTTGATAATGGTATCATTAGGGATGTAAATCCTGATGATA | |
| | AGTGCTTTGCTAATAAGCACCGGTCCTTCACACAATGGTATCATGAGCAT | |
| | GTTGGTGGTGTCTATGACAACTCTATCACATGCCCATTGACAGTTGCAGT | |
| | AATTGCTGGAGTTGCTGGTGCTCGCATTCCAGACGTACCTACTACATTGG | |
| | CTTGGGTGAACAATCAGATAATTTTCTTTGTTTCTCGAGTCTTTGCTAAT | |
| | ACAGGCAGTGTTTGCTACACTCCTATAGATGAGATACCCTATAAGAGTTT | |
| | CTCTGATAGTGGTTGCATTCTTCCATCTGAGTGCACTATGTTTAGGGATG | |
| | CAGAGGGCCGTATGACACCATACTGCCATGATCCTACTGTTTTGCCTGGG | |
| | GCTTTTGCGTACAGTCAGATGAGGCCTCATGTTCGTTACGACTTGTATGA | |
| | TGGTAACATGTTTATTAAATTTCCTGAAGTAGTATTTGAAAGTACACTTA | |
| | GGATTACTAGAACTCTGTCAACTCAGTACTGCCGGTTCGGTAGTTGTGAG | |
| | TATGCACAAGAGGGTGTTTGTATTACCACAAATGGCTCGTGGGCCATTTT | |
| | TAATGACCACCATCTTAATAGACCTGGTGTCTATTGTGGCTCTGATTTTA | |
| | TTGACATTGTCAGGCGGTTAGCAGTATCACTGTTCCAGCCTATTACTTAT | |
| | TTCCAATTGACTACCTCATTGGTCTTGGGTATAGGTTTGTGTGCGTTCCT | |
| | GACTTTGCTCTTCTATTATATTAATAAAGTAAAACGTGCTTTTGCAGATT | |
| | ACACCCAGTGTGCTGTAATTGCTGTTGTTGCTGCTGTTCTTAATAGCTTG | |
| | TGCATCTGCTTTGTTACCTCTATACCATTGTGTATAGTACCTTACACTGC | |
| | ATTGTACTATTATGCTACATTCTATTTTACTAATGAGCCTGCATTTATTA | |
| | TGCATGTTTCTTGGTACATTATGTTCGGGCCTATCGTTCCCATATGGATG | |
| | ACCTGCGTCTATACAGTTGCAATGTGCTTTAGACACTTCTTCTGGGTTTT | |
| | AGCTTATTTTAGTAAGAAACATGTAGAAGTTTTTACTGATGGTAAGCTTA | |
| | ATTGTAGTTTCCAGGACGCTGCCTCTAATATCTTTGTTATTAACAAGGAC | |
| | ACTTATGCAGCTCTTAGAAACTCTTTAACTAATGATGCCTATTCACGATT | |
| | TTTGGGGTTGTTTAACAAGTATAAGTACTTCTCTGGTGCTATGGAAACAG | |
| | CCGCTTATCGTGAAGCTGCAGCATGTCATCTTGCTAAAGCCTTACAAACA | |
| | TACAGCGAGACTGGTAGTGATCTTCTTTACCAACCACCCAACTGTAGCAT | |
| | AACCTCTGGCGTGTTGCAAAGCGGTTTGGTGAAAATGTCACATCCCAGTG | |
| | GAGATGTTGAGGCTTGTATGGTTCAGGTTACCTGCGGTAGCATGACTCTT | |
| | AATGGTCTTTGGCTTGACAACACAGTCTGGTGCCCACGACACGTAATGTG | |
| | CCCGGCTGACCAGTTGTCTGATCCTAATTATGATGCCTTGTTGATTTCTA | |
| | TGACTAATCATAGTTTCAGTGTGCAAAAACACATTGGCGCTCCAGCAAAC | |
| | TTGCGTGTTGTTGGTCATGCCATGCAAGGCACTCTTTTGAAGTTGACTGT | |
| | CGATGTTGCTAACCCTAGCACTCCAGCCTACACTTTTACAACAGTGAAAC | |
| | CTGGCGCAGCATTTAGTGTGTTAGCATGCTATAATGGTCGTCCGACTGGT | |
| | ACATTCACTGTTGTAATGCGCCCTAACTACACAATTAAGGGTTCCTTTCT | |
| | GTGTGGTTCTTGTGGTAGTGTTGGTTACACCAAGGAGGGTAGTGTGATCA | |
| | ATTTCTGTTACATGCATCAAATGGAACTTGCTAATGGTACACATACCGGT | |
| | TCAGCATTTGATGGTACTATGTATGGTGCCTTTATGGATAAACAAGTGCA | |
| | CCAAGTTCAGTTAACAGACAAATACTGCAGTGTTAATGTAGTAGCTTGGC | |
| | TTTACGCAGCAATACTTAATGGTTGCGCTTGGTTTGTAAAACCTAATCGC | |
| | ACTAGTGTTGTTTCTTTTAATGAATGGGCTCTTGCCAACCAATTCACTGA | |
| | ATTTGTTGGCACTCAATCCGTTGACATGTTAGCTGTCAAAACAGGCGTTG | |
| | CTATTGAACAGCTGCTTTATGCGATCCAACAACTGTATACTGGGTTCCAG | |
| | GGAAAGCAAATCCTTGGCAGTACCATGTTGGAAGATGAATTCACACCTGA | |
| | GGATGTTAATATGCAGATTATGGGTGTGGTTATGCAGAGTGGTGTGAGAA | |
| | AAGTTACATATGGTACTGCGCATTGGTTGTTTGCGACCCTTGTCTCAACC | |
| | TATGTGATAATCTTACAAGCCACTAAATTTACTTTGTGGAACTACTTGTT | |
| | TGAGACTATTCCCACACAGTTGTTCCCACTCTTATTTGTGACTATGGCCT | |
| | TCGTTATGTTGTTGGTTAAACACAAACACACCTTTTTGACACTTTTCTTG | |
| | TTGCCTGTGGCTATTTGTTTGACTTATGCAAACATAGTCTACGAGCCCAC | |
| | TACTCCCATTTCGTCAGCGCTGATTGCAGTTGCAAATTGGCTTGCCCCCA | |
| | CTAATGCTTATATGCGCACTACACATACTGATATTGGTGTCTACATTAGT | |
| | ATGTCACTTGTATTAGTCATTGTAGTGAAGAGATTGTACAACCCATCACT | |
| | TTCTAACTTTGCGTTAGCATTGTGCAGTGGTGTAATGTGGTTGTACACTT | |
| | ATAGCATTGGAGAAGCCTCAAGCCCCATTGCCTATCTGGTTTTTGTCACT | |
| | ACACTCACTAGTGATTATACGATTACAGTCTTTGTTACTGTCAACCTTGC | |
| | AAAAGTTTGCACTTATGCCATCTTTGCTTACTCACCACAGCTTACACTTG | |
| | TGTTTCCGGAAGTGAAGATGATACTTTTATTATACACATGTTTAGGTTTC | |
| | ATGTGTACTTGCTATTTTGGTGTCTTCTCTCTTTTGAACCTTAAGCTTAG | |
| | AGCACCTATGGGTGTCTATGACTTTAAGGTCTCAACACAAGAGTTCAGAT | |
| | TCATGACTGCTAACAATCTAACTGCACCTAGAAATTCTTGGGAGGCTATG | |
| | GCTCTGAACTTTAAGTTAATAGGTATTGGCGGTACACCTTGTATAAAGGT | |
| | TGCTGCTATGCAGTCTAAACTTACAGATCTTAAATGCACATCTGTGGTTC | |
| | TCCTCTCTGTGCTCCAACAGTTACACTTAGAGGCTAATAGTAGGGCCTGG | |
| | GCTTTCTGTGTTAAATGCCATAATGATATATTGGCAGCAACAGACCCCAG | |
| | TGAGGCTTTCGAGAAATTCGTAAGTCTCTTTGCTACTTTAATGACTTTTT | |
| | CTGGTAATGTAGATCTTGATGCGTTAGCTAGTGATATTTTTGACACTCCT | |
| | AGCGTACTTCAAGCTACTCTTTCTGAGTTTTCACACTTAGCTACCTTTGC | |
| | TGAGTTGGAAGCTGCGCAGAAAGCCTATCAGGAAGCTATGGACTCTGGTG | |
| | ACACCTCACCACAAGTTCTTAAGGCTTTGCAGAAGGCTGTTAATATAGCT | |
| | AAAAACGCCTATGAGAAGGATAAGGCAGTGGCCCGTAAGTTAGAACGTAT | |
| | GGCTGATCAGGCTATGACTTCTATGTATAAGCAAGCACGTGCTGAAGACA | |
| | AGAAAGCAAAAATTGTCAGTGCTATGCAAACTATGTTGTTTGGTATGATT | |
| | AAGAAGCTCGACAACGATGTTCTTAATGGTATCATTTCTAACGCTAGGAA | |
| | TGGTTGTATACCTCTTAGTGTCATCCCACTGTGTGCTTCAAATAAACTTC | |
| | GCGTTGTAATTCCTGACTTCACCGTCTGGAATCAGGTAGTCACATATCCC | |
| | TCGCTTAACTACGCTGGGGCTTTGTGGGACATTACAGTTATAAACAATGT | |
| | GGACAATGAAATTGTTAAGTCTTCAGATGTTGTAGACAGCAATGAAAATT | |
| | TAACATGGCCACTTGTTTTAGAATGCACTAGGGCATCCACTTCTGCCGTT | |
| | AAGTTGCAAAATAATGAGATCAAACCTTCAGGTCTAAAAACCATGGTTGT | |
| | GTCTGCGGGTCAAGAGCAAACTAACTGTAATACTAGTTCCTTAGCTTATT | |
| | ACGAACCTGTGCAGGGTCGTAAAATGCTGATGGCTCTTCTTTCTGATAAT | |
| | GCCTATCTCAAATGGGCGCGTGTTGAAGGTAAGGACGGATTTGTCAGTGT | |
| | AGAGCTACAACCTCCTTGCAAATTCTTGATTGCGGGACCAAAAGGACCTG | |
| | AAATCCGATATCTCTATTTTGTTAAAAATCTTAACAACCTTCATCGCGGG | |
| | CAAGTGTTAGGGCACATTGCTGCGACTGTTAGATTGCAAGCTGGTTCTAA | |
| | CACCGAGTTTGCCTCTAATTCCTCGGTGTTGTCACTTGTTAACTTCACCG | |
| | TTGATCCTCAAAAAGCTTATCTCGATTTCGTCAATGCGGGAGGTGCCCCA | |
| | TTGACAAATTGTGTTAAGATGCTTACTCCTAAAACTGGTACAGGTATAGC | |
| | TATATCTGTTAAACCAGAGAGTACAGCTGATCAAGAGACTTATGGTGGAG | |
| | CTTCAGTGTGTCTCTATTGCCGTGCGCATATAGAACATCCTGATGTCTCT | |
| | GGTGTTTGTAAATATAAGGGTAAGTTTGTCCAAATCCCTGCTCAGTGTGT | |
| | CCGTGACCCTGTGGGATTTTGTTTGTCAAATACCCCCTGTAATGTCTGTC | |
| | AATATTGGATTGGATATGGGTGCAATTGTGACTCGCTTAGGCAAGCAGCA | |
| | CTGCCCCAATCTAAAGATTCCAATTTTTTAAACGAGTCCGGGGTTCTATT | |
| | GTAAATGCCCGAATAGAACCCTGTTCAAGTGGTTTGTCCACTGATGTCGT | |
| | CTTTAGGGCATTTGACATCTGCAACTATAAGGCTAAGGTTGCTGGTATTG | |
| | GAAAATACTACAAGACTAATACTTGTAGGTTTGTAGAATTAGATGACCAA | |
| | GGGCATCATTTAGACTCCTATTTTGTCGTTAAGAGGCATACTATGGAGAA | |
| | TTATGAACTAGAGAAGCACTGTTACGACTTGTTACGTGACTGTGATGCTG | |
| | TAGCTCCCCATGATTTCTTCATCTTTGATGTAGACAAAGTTAAAACACCT | |
| | CATATTGTACGTCAGCGTTTAACTGAGTACACTATGATGGATCTTGTATA | |
| | TGCCCTGAGGCACTTTGATCAAAATAGCGAAGTGCTTAAGGCTATCTTAG | |
| | TGAAGTATGGTTGCTGTGATGTTACCTACTTTGAAAATAAACTCTGGTTT | |
| | GATTTTGTTGAAAATCCCAGTGTTATTGGTGTTTATCATAAACTTGGAGA | |
| | ACGTGTACGCCAAGCTATCTTAAACACTGTTAAATTTTGTGACCACATGG | |
| | TCAAGGCTGGTTTAGTCGGTGTGCTCACACTAGACAACCAGGACCTTAAT | |
| | GGCAAGTGGTATGATTTTGGTGACTTCGTAATCACTCAACCTGGTTCAGG | |
| | AGTAGCTATAGTTGATAGCTACTATTCTTATTTGATGCCTGTGCTCTCAA | |
| | TGACCGATTGTCTGGCCGCTGAGACACATAGGGATTGTGATTTTAATAAA | |
| | CCACTCATTGAGTGGCCACTTACTGAGTATGATTTTACTGATTATAAGGT | |
| | ACAACTCTTTGAGAAGTACTTTAAATATTGGGATCAGACGTATCACGCAA | |
| | ATTGCGTTAATTGTACTGATGACCGTTGTGTGTTACATTGTGCTAATTTC | |
| | AATGTATTGTTTGCTATGACCATGCCTAAGACTTGTTTCGGACCCATAGT | |
| | CCGAAAGATCTTTGTTGATGGCGTGCCATTTGTAGTATCTTGTGGTTATC | |
| | ACTACAAAGAATTAGGTTTAGTCATGAATATGGATGTTAGTCTCCATAGA | |
| | CATAGGCTCTCTCTTAAGGAGTTGATGATGTATGCCGCTGATCCAGCCAT | |
| | GCACATTGCCTCCTCTAACGCTTTTCTTGATTTGAGGACATCATGTTTTA | |
| | GTGTCGCTGCACTTACAACTGGTTTGACTTTTCAAACTGTGCGGCCTGGC | |
| | AATTTTAACCAAGACTTCTATGATTTCGTGGTATCTAAAGGTTTCTTTAA | |
| | GGAGGGCTCTTCAGTGACGCTCAAACATTTTTTCTTTGCTCAAGATGGTA | |
| | ATGCTGCTATTACAGATTATAATTACTATTCTTATAATCTGCCTACTATG | |
| | TGTGACATCAAACAAATGTTGTTCTGCATGGAAGTTGTAAACAAGTACTT | |
| | CGAAATCTATGACGGTGGTTGTCTTAATGCTTCTGAAGTGGTTGTTAATA | |
| | ATTTAGACAAGAGTGCTGGCCATCCTTTTAATAAGTTTGGCAAAGCTCGT | |
| | GTCTATTATGAGAGCATGTCTTACCAGGAGCAAGATGAACTTTTTGCCAT | |
| | GACAAAGCGTAACGTCATTCCTACCATGACTCAAATGAATCTAAAATATG | |
| | CTATTAGTGCTAAGAATAGAGCTCGCACTGTTGCAGGCGTGTCCATACTT | |
| | AGCACAATGACTAATCGCCAGTACCATCAGAAAATGCTTAAGTCCATGGC | |
| | TGCAACTCGTGGAGCGACTTGCGTCATTGGTACTACAAAGTTCTACGGTG | |
| | GCTGGGATTTCATGCTTAAAACATTGTACAAAGATGTTGATAATCCGCAT | |
| | CTTATGGGTTGGGATTACCCTAAGTGTGATAGAGCTATGCCTAATATGTG | |
| | TAGAATCTTCGCTTCACTCATATTAGCTCGTAAACATGGCACTTGTTGTA | |
| | CTACAAGGGACAGATTTTATCGCTTGGCAAATGAGTGTGCTCAGGTGCTA | |
| | AGCGAATATGTTCTATGTGGTGGTGGTTACTACGTCAAACCTGGAGGTAC | |
| | CAGTAGCGGAGATGCCACCACTGCATATGCCAATAGTGTCTTTAACATTT | |
| | TGCAGGCGACAACTGCTAATGTCAGTGCACTTATGGGTGCTAATGGCAAC | |
| | AAGATTGTTGACAAAGAAGTTAAAGACATGCAGTTTGATTTGTATGTCAA | |
| | TGTTTACAGGAGCACTAGCCCAGACCCCAAATTTGTTGATAAATACTATG | |
| | CTTTTCTTAATAAGCACTTTTCTATGATGATACTGTCTGATGACGGTGTC | |
| | GTTTGCTATAATAGTGATTATGCAGCTAAGGGTTACATTGCTGGAATACA | |
| | GAATTTTAAGGAAACGCTGTATTATCAGAACAATGTCTTTATGTCTGAAG | |
| | CTAAATGCTGGGTGGAAACCGATCTGAAGAAAGGGCCACATGAATTCTGT | |
| | TCACAGCATACGCTTTATATTAAGGATGGCGACGATGGTTACTTCCTTCC | |
| | TTATCCAGACCCTTCAAGAATTTTGTCTGCCGGTTGCTTTGTAGATGATA | |
| | TCGTTAAGACTGACGGTACACTCATGGTAGAGCGGTTTGTGTCTTTGGCT | |
| | ATAGATGCTTACCCTCTCACAAAGCATGAAGATATAGAATACCAGAATGT | |
| | ATTCTGGGTCTACTTACAGTATATAGAAAAACTGTATAAAGACCTTACAG | |
| | GACACATGCTTGACAGTTATTCTGTCATGCTATGTGGTGATAATTCTGCT | |
| | AAGTTTTGGGAAGAGGCATTCTATAGAGATCTCTATAGTTCGCCTACCAC | |
| | TTTGCAGGCTGTCGGTTCATGCGTTGTATGCCATTCACAGACTTCCCTAC | |
| | GCTGTGGGACATGCATCCGTAGACCATTTCTCTGCTGTAAATGCTGCTAT | |
| | GATCATGTTATAGCAACTCCACATAAGATGGTTTTGTCTGTTTCTCCTTA | |
| | CGTTTGTAATGCCCCTGGTTGTGGCGTTTCAGACGTTACTAAGCTATATT | |
| | TAGGTGGTATGAGCTACTTTTGTGTAGATCATAGACCTGTGTGTAGTTTT | |
| | CCACTTTGCGCTAATGGTCTTGTATTCGGCTTATACAAGAATATGTGCAC | |
| | AGGTAGTCCTTCTATAGTTGAATTTAATAGGTTGGCTACCTGTGACTGGA | |
| | CTGAAAGTGGTGATTACACCCTTGCCAATACTACAACAGAACCACTCAAA | |
| | CTTTTTGCTGCTGAGACTTTACGTGCCACTGAAGAGGCGTCTAAGCAGTC | |
| | TTATGCTATTGCCACCATCAAAGAAATTGTTGGTGAGCGCCAACTATTAC | |
| | TTGTGTGGGAGGCTGGCAAGTCCAAACCACCACTCAATCGTAATTATGTT | |
| | TTTACTGGTTATCATATAACCAAAAATAGTAAAGTGCAGCTCGGTGAGTA | |
| | CATTTTCGAGCGCATTGATTATAGTGATGCTGTATCCTACAAGTCTAGTA | |
| | CAACGTATAAACTGACTGTAGGTGACATCTTCGTACTTACCTCTCACTCT | |
| | GTGGCTACCTTGACGGCGCCCACAATTGTGAATCAAGAGAGGTATGTTAA | |
| | AATTACTGGGTTGTACCCAACCATTACGGTACCTGAAGAGTTCGCAAGTC | |
| | ATGTTGCCAACTTCCAAAAATCAGGTTATAGTAAATATGTCACTGTTCAG | |
| | GGACCACCTGGCACTGGCAAAAGTCATTTTGCTATAGGGTTAGCGATTTA | |
| | CTACCCTACAGCACGTGTTGTTTATACAGCATGTTCACACGCAGCTGTTG | |
| | ATGCTTTGTGTGAAAAAGCTTTTAAATATTTGAACATTGCTAAATGTTCC | |
| | CGTATCATTCCTGCAAAGGCACGTGTTGAGTGCTATGACAGGTTTAAAGT | |
| | TAATGAGACAAATTCTCAATATTTGTTTAGTACTATTAATGCTCTACCAG | |
| | AAACTTCTGCCGATATTCTGGTGGTTGATGAGGTTAGTATGTGCACTAAT | |
| | TATGATCTTTCAATTATTAATGCACGTATTAAAGCTAAGCACATTGTCTA | |
| | TGTAGGAGATCCAGCACAGTTGCCAGCTCCTAGGACTTTGTTGACTAGAG | |
| | GCACATTGGAACCAGAAAATTTCAATAGTGTCACTAGATTGATGTGTAAC | |
| | TTAGGTCCTGACATATTTTTAAGTATGTGCTACAGGTGTCCTAAGGAAAT | |
| | AGTAAGCACTGTGAGCGCTCTTGTCTACAATAATAAATTGTTAGCCAAGA | |
| | AGGAGCTTTCAGGCCAGTGCTTTAAAATACTCTATAAGGGCAATGTGACG | |
| | CATGATGCTAGCTCTGCCATTAATAGACCACAACTCACATTTGTGAAGAA | |
| | TTTTATTACTGCCAATCCGGCATGGAGTAAGGCAGTCTTTATTTCGCCTT | |
| | ACAATTCACAGAATGCTGTGTCTCGTTCAATGCTGGGTCTTACCACTCAG | |
| | ACTGTTGATTCCTCACAGGGTTCAGAATACCAGTACGTTATCTTCTGTCA | |
| | AACAGCAGATACGGCACATGCTAACAACATTAACAGATTTAATGTTGCAA | |
| | TCACTCGTGCCCAAAAAGGTATTCTTTGTGTTATGACATCTCAGGCACTC | |
| | TTTGAGTCCTTAGAGTTTACTGAATTGTCTTTTACTAATTACAAGCTCCA | |
| | GTCTCAGATTGTAACTGGCCTTTTTAAAGATTGCTCTAGAGAAACTTCTG | |
| | GCCTCTCACCTGCTTATGCACCAACATATGTTAGTGTTGATGACAAGTAT | |
| | AAGACGAGTGATGAGCTTTGCGTGAATCTTAATTTACCCGCAAATGTCCC | |
| | ATACTCTCGTGTTATTTCCAGGATGGGCTTTAAACTCGATGCAACAGTTC | |
| | CTGGATATCCTAAGCTTTTCATTACTCGTGAAGAGGCTGTAAGGCAAGTT | |
| | CGAAGCTGGATAGGCTTCGATGTTGAGGGTGCTCATGCTTCCCGTAATGC | |
| | ATGTGGCACCAATGTGCCTCTACAATTAGGATTTTCAACTGGTGTGAACT | |
| | TTGTTGTTCAGCCAGTTGGTGTTGTAGACACTGAGTGGGGTAACATGTTA | |
| | ACGGGCATTGCTGCACGTCCTCCACCAGGTGAACAGTTTAAGCACCTCGT | |
| | GCCTCTTATGCATAAGGGGGCTGCGTGGCCTATTGTTAGACGACGTATAG | |
| | TGCAAATGTTGTCAGACACTTTAGACAAATTGTCTGATTACTGTACGTTT | |
| | GTTTGTTGGGCTCATGGCTTTGAATTAACGTCTGCATCATACTTTTGCAA | |
| | GATAGGTAAGGAACAGAAGTGTTGCATGTGCAATAGACGCGCTGCAGCGT | |
| | ACTCTTCACCTCTGCAATCTTATGCCTGCTGGACTCATTCCTGCGGTTAT | |
| | GATTATGTCTACAACCCTTTCTTTGTCGATGTTCAACAGTGGGGTTATGT | |
| | AGGCAATCTTGCTACTAATCACGATCGTTATTGCTCTGTCCATCAAGGAG | |
| | CTCATGTGGCTTCTAATGATGCAATAATGACTCGTTGTTTAGCTATTCAT | |
| | TCTTGTTTTATAGAACGTGTGGATTGGGATATAGAGTATCCTTATATCTC | |
| | ACATGAAAAGAAATTGAATTCCTGTTGTAGAATCGTTGAGCGCAACGTCG | |
| | TACGTGCTGCTCTTCTTGCCGGTTCATTTGACAAAGTCTATGATATTGGC | |
| | AATCCTAAAGGAATTCCTATTGTTGATGACCCTGTGGTTGATTGGCATTA | |
| | TTTTGATGCACAGCCCTTGACCAGGAAGGTACAACAGCTTTTCTATACAG | |
| | AGGACATGGCCTCAAGATTTGCTGATGGGCTCTGCTTATTTTGGAACTGT | |
| | AATGTACCAAAATATCCTAATAATGCAATTGTATGCAGGTTTGACACACG | |
| | TGTGCATTCTGAGTTCAATTTGCCAGGTTGTGATGGCGGTAGTTTGTATG | |
| | TTAACAAGCACGCTTTTCATACACCAGCATATGATGTGAGTGCATTCCGT | |
| | GATCTGAAACCTTTACCATTCTTTTATTATTCTACTACACCATGTGAAGT | |
| | GCATGGTAATGGTAGTATGATAGAGGATATTGATTATGTACCCCTAAAAT | |
| | CTGCAGTCTGTATTACAGCTTGTAATTTAGGGGGCGCTGTTTGTAGGAAG | |
| | CATGCTACAGAGTACAGAGAGTATATGGAAGCATATAATCTTGTCTCTGC | |
| | ATCAGGTTTCCGCCTTTGGTGTTATAAGACCTTTGATATTTATAATCTCT | |
| | GGTCTACTTTTACAAAAGTTCAAGGTTTGGAAAACATTGCTTTTAATGTT | |
| | GTTAAACAAGGCCATTTTATTGGTGTTGAGGGTGAACTACCTGTAGCTGT | |
| | AGTCAATGATAAGATCTTCACCAAGAGTGGCGTTAATGACATTTGTATGT | |
| | TTGAGAATAAAACCACTTTGCCTACTAATATAGCTTTTGAACTCTATGCT | |
| | AAGCGTGCTGTACGCTCGCATCCCGATTTCAAATTGCTACACAATTTACA | |
| | AGCAGACATTTGCTACAAGTTCGTCCTTTGGGATTATGAACGTAGCAATA | |
| | TTTATGGTACTGCTACTATTGGTGTATGTAAGTACACTGATATTGATGTT | |
| | AATTCAGCTTTGAATATATGTTTTGACATACGCGATAATTGTTCATTGGA | |
| | GAAGTTCATGTCTACTCCCAATGCCATCTTTATTTCTGATAGAAAAATCA | |
| | AGAAATACCCTTGTATGGTAGGTCCTGATTATGCTTACTTCAATGGTGCT | |
| | ATCATCCGTGATAGTGATGTTGTTAAACAACCAGTGAAGTTCTACTTGTA | |
| | TAAGAAAGTCAATAATGAGTTTATTGATCCTACTGAGTGTATTTACACTC | |
| | AGAGTCGCTCTTGTAGTGACTTCCTACCCCTTTCTGACATGGAGAAAGAC | |
| | TTTCTATCTTTTGATAGTGATGTTTTCATTAAGAAGTATGGCTTGGAAAA | |
| | CTATGCTTTTGAGCACGTAGTCTATGGAGACTTCTCTCATACTACGTTAG | |
| | GCGGTCTTCACTTGCTTATTGGTTTATACAAGAAGCAACAGGAAGGTCAT | |
| | ATTATTATGGAAGAAATGCTAAAAGGTAGCTCAACTATTCATAACTATTT | |
| | TATTACTGAGACTAACACAGCGGCTTTTAAGGCGGTGTGTTCTGTTATAG | |
| | ATTTAAAGCTTGACGACTTTGTTATGATTTTAAAGAGTCAAGACCTTGGC | |
| | GTAGTATCCAAGGTTGTCAAGGTTCCTATTGACTTAACAATGATTGAGTT | |
| | TATGTTATGGTGTAAGGATGGACAGGTTCAAACCTTCTACCCTCGACTCC | |
| | AGGCTTCTGCAGATTGGAAACCTGGTCATGCAATGCCATCCCTCTTTAAA | |
| | GTTCAAAATGTAAACCTTGAACGTTGTGAGCTTGCTAATTACAAGCAATC | |
| | TATTCCTATGCCTCGCGGTGTGCACATGAACATCGCTAAATATATGCAAT | |
| | TGTGCCAGTATTTAAATACTTGCACATTAGCCGTGCCTGCCAATATGCGT | |
| | GTTATACATTTTGGCGCTGGTTCTGATAAAGGTATCGCTCCTGGTACCTC | |
| | AGTTTTACGACAGTGGCTTCCTACAGATGCCATTATTATAGATAATGATT | |
| | TAAATGAGTTCGTGTCAGATGCTGACATAACTTTATTTGGAGATTGTGTA | |
| | ACTGTACGTGTCGGCCAACAAGTGGATCTTGTTATTTCCGACATGTATGA | |
| | TCCTACTACTAAGAATGTAACAGGTAGTAATGAGTCAAAGGCTTTATTCT | |
| | TTACTTACCTGTGTAACCTCATTAATAATAATCTTGCTCTTGGTGGGTCT | |
| | GTTGCTATTAAAATAACAGAACACTCTTGGAGCGTTGAACTTTATGAACT | |
| | TATGGGAAAATTTGCTTGGTGGACTGTTTTCTGCACCAATGCAAATGCAT | |
| | CCTCATCTGAAGGATTCCTCTTAGGTATTAATTACTTGGGTACTATTAAA | |
| | GAAAATATAGATGGTGGTGCTATGCACGCCAACTATATATTTTGGAGAAA | |
| | TTCCACTCCTATGAATCTGAGTACTTACTCACTTTTTGATTTATCCAAGT | |
| | TTCAATTAAAATTAAAAGGAACACCAGTTCTTCAATTAAAGGAGAGTCAA | |
| | ATTAACGAACTCGTAATATCTCTCCTGTCGCAGGGTAAGTTACTTATCCG | |
| | TGACAATGATACACTCAGTGTTTCTACTGATGTTCTTGTTAACACCTACA | |
| | GAAAGTTACGTTGATGTAGGGCCAGATTCTGTTAAGTCTGCTTGTATTGA | |
| | GGTTGATATACAACAGACTTTCTTTGATAAAACTTGGCCTAGGCCAATTG | |
| | ATGTTTCTAAGGCTGACGGTATTATATACCCTCAAGGCCGTACATATTCT | |
| | AACATAACTATCACTTATCAAGGTCTTTTTCCCTATCAGGGAGACCATGG | |
| | TGATATGTATGTTTACTCTGCAGGACATGCTACAGGCACAACTCCACAAA | |
| | AGTTGTTTGTAGCTAACTATTCTCAGGACGTCAAACAGTTTGCTAATGGG | |
| | TTTGTCGTCCGTATAGGAGCAGCTGCCAATTCCACTGGCACTGTTATTAT | |
| | TAGCCCATCTACCAGCGCTACTATACGAAAAATTTACCCTGCTTTTATGC | |
| | TGGGTTCTTCAGTTGGTAATTTCTCAGATGGTAAAATGGGCCGCTTCTTC | |
| | AATCATACTCTAGTTCTTTTGCCCGATGGATGTGGCACTTTACTTAGAGC | |
| | TTTTTATTGTATTCTAGAGCCTCGCTCTGGAAATCATTGTCCTGCTGGCA | |
| | ATTCCTATACTTCTTTTGCCACTTATCACACTCCTGCAACAGATTGTTCT | |
| | GATGGCAATTACAATCGTAATGCCAGTCTGAACTCTTTTAAGGAGTATTT | |
| | TAATTTACGTAACTGCACCTTTATGTACACTTATAACATTACCGAAGATG | |
| | AGATTTTAGAGTGGTTTGGCATTACACAAACTGCTCAAGGTGTTCACCTC | |
| | TTCTCATCTCGGTATGTTGATTTGTACGGCGGCAATATGTTTCAATTTGC | |
| | CACCTTGCCTGTTTATGATACTATTAAGTATTATTCTATCATTCCTCACA | |
| | GTATTCGTTCTATCCAAAGTGATAGAAAAGCTTGGGCTGCCTTCTACGTA | |
| | TATAAACTTCAACCGTTAACTTTCCTGTTGGATTTTTCTGTTGATGGTTA | |
| | TATACGCAGAGCTATAGACTGTGGTTTTAATGATTTGTCACAACTCCACT | |
| | GCTCATATGAATCCTTCGATGTTGAATCTGGAGTTTATTCAGTTTCGTCT | |
| | TTCGAAGCAAAACCTTCTGGCTCAGTTGTGGAACAGGCTGAAGGTGTTGA | |
| | ATGTGATTTTTCACCTCTTCTGTCTGGCACACCTCCTCAGGTTTATAATT | |
| | TCAAGCGTTTGGTTTTTACCAATTGCAATTATAATCTTACCAAATTGCTT | |
| | TCACTTTTTTCTGTGAATGATTTTACTTGTAGTCAAATATCTCCAGCAGC | |
| | AATTGCTAGCAACTGTTATTCTTCACTGATTTTGGATTACTTTTCATACC | |
| | CACTTAGTATGAAATCCGATCTCAGTGTTAGTTCTGCTGGTCCAATATCC | |
| | CAGTTTAATTATAAACAGTCCTTTTCTAATCCCACATGTTTGATTTTAGC | |
| | GACTGTTCCTCATAACCTTACTACTATTACTAAGCCTCTTAAGTACAGCT | |
| | ATATTAACAAGTGCTCTCGTCTTCTTTCTGATGATCGTACTGAAGTACCT | |
| | CAGTTAGTGAACGCTAATCAATACTCACCCTGTGTATCCATTGTCCCATC | |
| | CACTGTGTGGGAAGACGGTGATTATTATAGGAAACAACTATCTCCACTTG | |
| | AAGGTGGTGGCTGGCTTGTTGCTAGTGGCTCAACTGTTGCCATGACTGAG | |
| | CAATTACAGATGGGCTTTGGTATTACAGTTCAATATGGTACAGACACCAA | |
| | TAGTGTTTGCCCCAAGCTTGAATTTGCTAATGACACAAAAATTGCCTCTC | |
| | AATTAGGCAATTGCGTGGAATATTCCCTCTATGGTGTTTCGGGCCGTGGT | |
| | GTTTTTCAGAATTGCACAGCTGTAGGTGTTCGACAGCAGCGCTTTGTTTA | |
| | TGATGCGTACCAGAATTTAGTTGGCTATTATTCTGATGATGGCAACTACT | |
| | ACTGTTTGCGTGCTTGTGTTAGTGTTCCTGTTTCTGTCATCTATGATAAA | |
| | GAAACTAAAACCCACGCTACTCTATTTGGTAGTGTTGCATGTGAACACAT | |
| | TTCTTCTACCATGTCTCAATACTCCCGTTCTACGCGATCAATGCTTAAAC | |
| | GGCGAGATTCTACATATGGCCCCCTTCAGACACCTGTTGGTTGTGTCCTA | |
| | GGACTTGTTAATTCCTCTTTGTTCGTAGAGGACTGCAAGTTGCCTCTTGG | |
| | TCAATCTCTCTGTGCTCTTCCTGACACACCTAGTACTCTCACACCTCGCA | |
| | GTGTGCGCTCTGTTCCAGGTGAAATGCGCTTGGCATCCATTGCTTTTAAT | |
| | CATGCTATTCAGGTTGATCAACTTAATAGTAGTTATTTTAAATTAAGTAT | |
| | ACCCACTAATTTTTCCTTTGGTGTGACTCAGGAGTACATTCAGACAACCA | |
| | TTCAGAAAGTTACTGTTGATTGTAAACAGTACGTTTGCAATGGTTTCCAG | |
| | AAGTGTGAGCAATTACTGCGCGAGTATGGCCAGTTTTGTTCCAAAATAAA | |
| | CCAGGCTCTCCATGGTGCCAATTTACGCCAGGATGATTCTGTACGTAATT | |
| | TGTTTGCGAGCGTGAAAAGCTCTCAATCATCTCCTATCATACCAGGTTTT | |
| | GGAGGTGACTTTAATTTGACACTTCTAGAACCTGTTTCTATATCTACTGG | |
| | CAGTCGTAGTGCACGTAGTGCTATTGAGGATTTGCTATTTGACAAAGTCA | |
| | CTATAGCTGATCCTGGTTATATGCAAGGTTACGATGATTGCATGCAGCAA | |
| | GGTCCAGCATCAGCTCGTGATCTTATTTGTGCTCAATATGTGGCTGGTTA | |
| | CAAAGTATTACCTCCTCTTATGGATGTTAATATGGAAGCCGCGTATACTT | |
| | CATCTTTGCTTGGCAGCATAGCAGGTGTTGGCTGGACTGCTGGCTTATCC | |
| | TCCTTTGCTGCTATTCCATTTGCACAGAGTATCTTTTATAGGTTAAACGG | |
| | TGTTGGCATTACTCAACAGGTTCTTTCAGAGAACCAAAAGCTTATTGCCA | |
| | ATAAGTTTAATCAGGCTCTGGGAGCTATGCAAACAGGCTTCACTACAACT | |
| | AATGAAGCTTTTCAGAAGGTTCAGGATGCTGTGAACAACAATGCACAGGC | |
| | TCTATCCAAATTAGCTAGCGAGCTATCTAATACTTTTGGTGCTATTTCCG | |
| | CCTCTATTGGAGACATCATACAACGTCTTGATGTTCTCGAACAGGACGCC | |
| | CAAATAGACAGACTTATTAATGGCCGTTTGACAACACTAAATGCTTTTGT | |
| | TGCACAGCAGCTTGTTCGTTCCGAATCAGCTGCTCTTTCCGCTCAATTGG | |
| | CTAAAGATAAAGTCAATGAGTGTGTCAAGGCACAATCCAAGCGTTCTGGA | |
| | TTTTGCGGTCAAGGCACACATATAGTGTCCTTTGTTGTAAATGCCCCTAA | |
| | TGGCCTTTACTTCATGCATGTTGGTTATTACCCTAGCAACCACATTGAGG | |
| | TTGTTTCTGCTTATGGTCTTTGCGATGCAGCTAACCCTACTAATTGTATA | |
| | GCCCCTGTTAATGGCTACTTTATTAAAACTAATAACACTAGGATTGTTGA | |
| | TGAGTGGTCATATACTGGCTCGTCCTTCTATGCACCTGAGCCCATTACCT | |
| | CCCTTAATACTAAGTATGTTGCACCACAGGTGACATACCAAAACATTTCT | |
| | ACTAACCTCCCTCCTCCTCTTCTCGGCAATTCCACCGGGATTGACTTCCA | |
| | AGATGAGTTGGATGAGTTTTTCAAAAATGTTAGCACCAGTATACCTAATT | |
| | TTGGTTCCCTAACACAGATTAATACTACATTACTCGATCTTACCTACGAG | |
| | ATGTTGTCTCTTCAACAAGTTGTTAAAGCCCTTAATGAGTCTTACATAGA | |
| | CCTTAAAGAGCTTGGCAATTATACTTATTACAACAAATGGCCGTGGTACA | |
| | TTTGGCTTGGTTTCATTGCTGGGCTTGTTGCCTTAGCTCTATGCGTCTTC | |
| | TTCATACTGTGCTGCACTGGTTGTGGCACAAACTGTATGGGAAAACTTAA | |
| | GTGTAATCGTTGTTGTGATAGATACGAGGAATACGACCTCGAGCCGCATA | |
| | AGGTTCATGTTCACTAATTAACGAACTATTAATGAGAGTTCAAAGACGAG | |
| | CCACTCTCTTGTTAGTGTTTTCACTCTCTCTTTTGGTCACTGCATCCTCA | |
| | AAACCTCTCTATGTACCTGAGCATTGTCAGAATTATTCTGGTTGCATGCT | |
| | TAGGGCTTGTATTAAAACTGCCCAAGCTGATACAGCTGGTCTTTATACAA | |
| | ATTTTCGAATTGACGTCCCATCTGCAGAATCAACTGGTACTCAATCAGTT | |
| | TCTGTCGATCTTGAGTCAACTTCAACTCATGATGGTCCTACCGAACATGT | |
| | TACTAGTGTGAATCTTTTTGACGTTGGTTACTCAGTTAATTAACGAACTC | |
| | TATGGATTACGTGTCTCTGCTTAATCAAATTTGGCAGAAGTACCTTAACT | |
| | CACCGTATACTACTTGTTTGTACATCCCTAAACCCACAGCTAAGTATACA | |
| | CCTTTAGTTGGCACTTCATTGCACCCTGTGCTGTGGAACTGTCAGCTATC | |
| | CTTTGCTGGTTATACTGAATCTGCTGTTAATTCTACAAAAGCTTTGGCCA | |
| | AACAGGACGCAGCTCAGCGAATCGCTTGGTTGCTACATAAGGATGGAGGA | |
| | ATCCCTGATGGATGTTCCCTCTACCTCCGGCACTCAAGTTTATTCGCGCA | |
| | AAGCGAGGAAGAGGAGCCATTCTCCAACTAAGAAACTGCGCTACGTTAAG | |
| | CGTAGATTTTCTCTTCTGCGCCATGAAGACCTTAGTGTTATTGTCCAACC | |
| | AACACACTATGTCAGGGTTACATTTTCAGACCCCAACATGTGGTATCTAC | |
| | GTTCGGGTCATCATTTACACTCAGTTCACAATTGGCTTAAACCTTATGGC | |
| | GGCCAACCTGTTTCTGAGTACCATATTACTCTAGCTTTGCTAAATCTCAC | |
| | TGATGAAGATTTAGCTAGAGATTTTTCACCCATTGCGCTCTTTTTGCGCA | |
| | ATGTCAGATTTGAGCTACATGAGTTCGCCTTGCTGCGCAAAACTCTTGTT | |
| | CTTAATGCATCAGAGATCTACTGTGCTAACATACATAGATTTAAGCCTGT | |
| | GTATAGAGTTAACACGGCAATCCCTACTATTAAGGATTGGCTTCTCGTTC | |
| | AGGGATTTTCCCTTTACCATAGTGGCCTCCCTTTACATATGTCAATCTCT | |
| | AAATTGCATGCACTGGATGATGTTACTCGCAATTACATCATTACAATGCC | |
| | ATGCTTTAGAACTTACCCTCAACAAATGTTTGTTACTCCTTTGGCCGTAG | |
| | ATGTTGTCTCCATACGGTCTTCCAATCAGGGTAATAAACAAATTGTTCAT | |
| | TCTTATCCCATTTTACATCATCCAGGATTTTAACGAACTATGGCTTTCTC | |
| | GGCGTCTTTATTTAAACCCGTCCAGCTAGTCCCAGTTTCTCCTGCATTTC | |
| | ATCGCATTGAGTCTACTGACTCTATTGTTTTCACATACATTCCTGCTAGC | |
| | GGCTATGTAGCTGCTTTAGCTGTCAATGTGTGTCTCATTCCCCTATTATT | |
| | ACTGCTACGTCAAGATACTTGTCGTCGCAGCATTATCAGAACTATGGTTC | |
| | TCTATTTCCTTGTTCTGTATAACTTTTTATTAGCCATTGTACTAGTCAAT | |
| | GGTGTACATTATCCAACTGGAAGTTGCCTGATAGCCTTCTTAGTTATCCT | |
| | CATAATACTTTGGTTTGTAGATAGAATTCGTTTCTGTCTCATGCTGAATT | |
| | CCTACATTCCACTGTTTGACATGCGTTCCCACTTTATTCGTGTTAGTACA | |
| | GTTTCTTCTCATGGTATGGTCCCTGTAATACACACCAAACCATTATTTAT | |
| | TAGAAACTTCGATCAGCGTTGCAGCTGTTCTCGTTGTTTTTATTTGCACT | |
| | CTTCCACTTATATAGAGTGCACTTATATTAGCCGTTTTAGTAAGATTAGC | |
| | CTAGTTTCTGTAACTGACTTCTCCTTAAACGGCAATGTTTCCACTGTTTT | |
| | CGTGCCTGCAACGCGCGATTCAGTTCCTCTTCACATAATCGCCCCGAGCT | |
| | CGCTTATCGTTTAAGCAGCTCTGCGCTACTATGGGTCCCGTGTAGAGGCT | |
| | AATCCATTAGTCTCTCTTTGGACATATGGAAAACGAACTATGTTACCCTT | |
| | TGTCCAAGAACGAATAGGGTTGTTCATAGTAAACTTTTTCATTTTTACCG | |
| | TAGTATGTGCTATAACACTCTTGGTGTGTATGGCTTTCCTTACGGCTACT | |
| | AGATTATGTGTGCAATGTATGACAGGCTTCAATACCCTGTTAGTTCAGCC | |
| | CGCATTATACTTGTATAATACTGGACGTTCAGTCTATGTAAAATTCCAGG | |
| | ATAGTAAACCCCCTCTACCACCTGACGAGTGGGTTTAACGAACTCCTTCA | |
| | TAATGTCTAATATGACGCAACTCACTGAGGCGCAGATTATTGCCATTATT | |
| | AAAGACTGGAACTTTGCATGGTCCCTGATCTTTCTCTTAATTACTATCGT | |
| | ACTACAGTATGGATACCCATCCCGTAGTATGACTGTCTATGTCTTTAAAA | |
| | TGTTTGTTTTATGGCTCCTATGGCCATCTTCCATGGCGCTATCAATATTT | |
| | AGCGCCGTTTATCCAATTGATCTAGCTTCCCAGATAATCTCTGGCATTGT | |
| | AGCAGCTGTTTCAGCTATGATGTGGATTTCCTACTTTGTGCAGAGTATCC | |
| | GGCTGTTTATGAGAACTGGATCATGGTGGTCATTCAATCCTGAGACTAAT | |
| | TGCCTTTTGAACGTTCCATTTGGTGGTACAACTGTCGTACGTCCACTCGT | |
| | AGAGGACTCTACCAGTGTAACTGCTGTTGTAACCAATGGCCACCTCAAAA | |
| | TGGCTGGCATGCATTTCGGTGCTTGTGACTACGACAGACTTCCTAATGAA | |
| | GTCACCGTGGCCAAACCCAATGTGCTGATTGCTTTAAAAATGGTGAAGCG | |
| | GCAAAGCTACGGAACTAATTCCGGCGTTGCCATTTACCATAGATATAAGG | |
| | CAGGTAATTACAGGAGTCCGCCTATTACGGCGGATATTGAACTTGCATTG | |
| | CTTCGAGCTTAGGCTCTTTAGTAAGAGTATCTTAATTGATTTTAACGAAT | |
| | CTCAATTTCATTGTTATGGCATCCCCTGCTGCACCTCGTGCTGTTTCCTT | |
| | TGCCGATAACAATGATATAACAAATACAAACCTATCTCGAGGTAGAGGAC | |
| | GTAATCCAAAACCACGAGCTGCACCAAATAACACTGTCTCTTGGTACACT | |
| | GGGCTTACCCAACACGGGAAAGTCCCTCTTACCTTTCCACCTGGGCAGGG | |
| | TGTACCTCTTAATGCCAATTCTACCCCTGCGCAAAATGCTGGGTATTGGC | |
| | GGAGACAGGACAGAAAAATTAATACCGGGAATGGAATTAAGCAACTGGCT | |
| | CCCAGGTGGTACTTCTACTACACTGGAACTGGACCCGAAGCAGCACTCCC | |
| | ATTCCGGGCTGTTAAGGATGGCATCGTTTGGGTCCATGAAGATGGCGCCA | |
| | CTGATGCTCCTTCAACTTTTGGGACGCGGAACCCTAACAATGATTCAGCT | |
| | ATTGTTACACAATTCGCGCCCGGTACTAAGCTTCCTAAAAACTTCCACAT | |
| | TGAGGGGACTGGAGGCAATAGTCAATCATCTTCAAGAGCCTCTAGCTTAA | |
| | GCAGAAACTCTTCCAGATCTAGTTCACAAGGTTCAAGATCAGGAAACTCT | |
| | ACCCGCGGCACTTCTCCAGGTCCATCTGGAATCGGAGCAGTAGGAGGTGA | |
| | TCTACTTTACCTTGATCTTCTGAACAGACTACAAGCCCTTGAGTCTGGCA | |
| | AAGTAAAGCAATCGCAGCCAAAAGTAATCACTAAGAAAGATGCTGCTGCT | |
| | GCTAAAAATAAGATGCGCCACAAGCGCACTTCCACCAAAAGTTTCAACAT | |
| | GGTGCAAGCTTTTGGTCTTCGCGGACCAGGAGACCTCCAGGGAAACTTTG | |
| | GTGATCTTCAATTGAATAAACTCGGCACTGAGGACCCACGTTGGCCCCAA | |
| | ATTGCTGAGCTTGCTCCTACAGCCAGTGCTTTTATGGGTATGTCGCAATT | |
| | TAAACTTACCCATCAGAACAATGATGATCATGGCAACCCTGTGTACTTCC | |
| | TTCGGTACAGTGGAGCCATTAAACTTGACCCAAAGAATCCCAACTACAAT | |
| | AAGTGGTTGGAGCTTCTTGAGCAAAATATTGATGCCTACAAAACCTTCCC | |
| | TAAGAAGGAAAAGAAACAAAAGGCACCAAAAGAAGAATCAACAGACCAAA | |
| | TGTCTGAACCTCCAAAGGAGCAGCGTGTGCAAGGTAGCATCACTCAGCGC | |
| | ACTCGCACCCGTCCAAGTGTTCAGCCTGGTCCAATGATTGATGTTAACAC | |
| | TGATTAGTGTCACTCAAAGTAACAAGATCGCGGCAATCGTTTGTGTTTGG | |
| | CAACCCCATCTCACCATCGCTTGTCCACTCTTGCACAGAATGGAATCATG | |
| | TTGTAATTACAGTGCAATAAGGTAATTATAACCCATTTAATTGATAGCTA | |
| | TGCTTTATTAAAGTGTGTAGCTGTAGAGAGAATGTTAAAGACTGTCACCT | |
| | CTGCTTGATTGCAAGTGAACAGTGCCCCCCGGGAAGAGCTCTACAGTGTG | |
| | AAATGTAAATAAAAAATAGCTATTATTCAATTAGATTAGGCTAATTAGAT | |
| | GATTTGCAAAAAAAAAAAA | |
| IL-8 siRNA | CAAGGAAGTGCTAAAGAA | 80 |
| sense strand (A1 | | |
| siRNA, A4 | | |
| siRNA) | | |
| IL-8 siRNA | CAAGGAGTGCTAAAGAA | 81 |
| sense strand (A2 | | |
| siRNA, A3-1 | | |
| siRNA, A5-1 | | |
| siRNA) | | |
| IL-8 siRNA | GAGAGTGATTGAGAGTGG | 82 |
| sense strand | | |
| (A3-2 siRNA, | | |
| A5-2 siRNA) | | |
| IL-8 siRNA | GAGAGCTCTGTCTGGACC | 83 |
| sense strand | | |
| (A3-3 siRNA, | | |
| A5-3 siRNA) | | |
| IL-1beta siRNA | GAAAGATGATAAGCCCACTCT | 84 |
| sense strand (A6 | | |
| siRNA, A7-1 | | |
| siRNA) | | |
| IL-1beta siRNA | GGTGATGTCTGGTCCATATGA | 85 |
| sense strand | | |
| (A7-2 siRNA) | | |
| IL-1beta siRNA | GATGATAAGCCCACTCTA | 86 |
| sense strand | | |
| (A7-3 siRNA) | | |
| TNF-alpha | GGCGTGGAGCTGAGAGATAA | 87 |
| sense strand | | |
| (A8-1 siRNA) | | |
| TNF-alpha | GGGCCTGTACCTCATCTACT | 88 |
| sense strand | | |
| (A8-2 siRNA) | | |
| TNF-alpha | GGTATGAGCCCATCTATCT | 89 |
| sense strand | | |
| (A8-3 siRNA) | | |
| IL-17 | GCAATGAGGACCCTGAGAGAT | 90 |
| sense strand | | |
| (A8-4 siRNA) | | |
| IL-17 | GCTGATGGGAACGTGGACTA | 91 |
| sense strand | | |
| (A8-5 siRNA) | | |
| IL-17 | GGTCCTCAGATTACTACAA | 92 |
| sense strand | | |
| (A8-6 siRNA) | | |
| IL-6 | GCCCTGAGAAAGGAGACATGT | 93 |
| sense strand | | |
| (B1-1 siRNA) | | |
| IL-6 | GAGGAGACTTGCCTGGTGAAA | 94 |
| sense strand | | |
| (B1-2, B2, B15- | | |
| 1, B16-1, B17-1 | | |
| siRNA) | | |
| IL-6 | GAGGGCTCTTCGGCAAATGTA | 95 |
| sense strand | | |
| (B1-3 siRNA) | | |
| IL6R-alpha | GTGAGGAAGTTTCAGAACAGT | 96 |
| sense strand | | |
| (B3-1, B4 | | |
| siRNA) | | |
| IL6R-alpha | GAACGGTCAAAGACATTCACA | 97 |
| sense strand | | |
| (B3-2 siRNA) | | |
| IL6R-Beta | GGGAAGGTTACATCAGATCAT | 98 |
| sense strand | | |
| (B3-3, B5 | | |
| siRNA) | | |
| ACE2 | GCAGCTGAGGCCATTATATGA | 99 |
| sense strand | | |
| (B6-1, B7, B15- | | |
| 2, B16-2, Bl7-2 | | |
| siRNA) | | |
| ACE2 | GGACCCAGGAAATGTTCAGAA | 100 |
| sense strand | | |
| (B6-2 siRNA) | | |
| ACE2 | GGCTGAAAGACCAGAACAAGA | 101 |
| sense strand | | |
| (B6-3 siRNA) | | |
| SARS CoV- | GTGTGACCGAAAGGTAAGATG | 102 |
| 2_ORF1ab | | |
| sense strand | | |
| (B8-1, B14, | | |
| B18-1 siRNA) | | |
| SARS CoV- | TTTAAATATTGGGATCAGAC | 103 |
| 2_ORF1ab | | |
| sense strand | | |
| (B12-1 siRNA) | | |
| SARS CoV- | AAGAATAGAGCTCGCAC | 104 |
| 2_ORF1ab | | |
| sense strand | | |
| (B12-2, B13 | | |
| siRNA) | | |
| SARS CoV- | ACTGTTGATTCATCACAGGG | 105 |
| 2_ORF1ab | | |
| sense strand | | |
| (B12-3 siRNA) | | |
| SARS CoV- | GTTGCTGATTATTCTGTCCTA | 106 |
| 2_Spike Protein | | |
| sense strand | | |
| (B11-1, B19-1 | | |
| siRNA) | | |
| SARS CoV- | GAGGTGATGAAGTCAGACAAA | 107 |
| 2_Spike Protein | | |
| sense strand | | |
| (B8-2, B9, B11- | | |
| 2, B18-2, B19-2 | | |
| siRNA) | | |
| SARS CoV- | GCCGGTAGCACACCTTGTAAT | 108 |
| 2_Spike Protein | | |
| sense strand | | |
| (B11-3, B19-3 | | |
| siRNA) | | |
| SARS CoV- | GCAACTGAGGGAGCCTTGAAT | 109 |
| 2_Nucleocapsid | | |
| Protein sense | | |
| strand (B8-3, | | |
| B10, B15-3, | | |
| B16-3, B17-3, | | |
| Bl8-3 siRNA) | | |
| IL-8 siRNA | TTCTTTAGCACTTCCTTG | 110 |
| antisense strand | | |
| (A1 siRNA, A4 | | |
| siRNA) | | |
| IL-8 siRNA | TTCTTTAGCACTCCTTG | 111 |
| antisense strand | | |
| (A2 siRNA, A3- | | |
| 1 siRNA, A5-1 | | |
| siRNA) | | |
| IL-8 siRNA | CCACTCTCAATCACTCTC | 112 |
| antisense strand | | |
| (A3-2 siRNA, | | |
| A5-2 siRNA) | | |
| IL-8 siRNA | GGTCCAGACAGAGCTCTC | 113 |
| antisense strand | | |
| (A3-3 siRNA, | | |
| A5-3 siRNA) | | |
| IL-1beta siRNA | AGAGTGGGCTTATCATCTTTC | 114 |
| antisense strand | | |
| (A6 siRNA, A7- | | |
| 1 siRNA) | | |
| IL-1beta siRNA | TCATATGGACCAGACATCACC | 115 |
| antisense strand | | |
| (A7-2 siRNA) | | |
| IL-1beta siRNA | TAGAGTGGGCTTATCATC | 116 |
| antisense strand | | |
| (A7-3 siRNA) | | |
| TNF-alpha | TTATCTCTCAGCTCCACGCC | 117 |
| antisense strand | | |
| (A8-1 siRNA) | | |
| TNF-alpha | AGTAGATGAGGTACAGGCCC | 118 |
| antisense strand | | |
| (A8-2 siRNA) | | |
| TNF-alpha | AGATAGATGGGCTCATACC | 119 |
| antisense strand | | |
| (A8-3 siRNA) | | |
| IL-17 | ATCTCTCAGGGTCCTCATTGC | 120 |
| antisense strand | | |
| (A8-4 siRNA) | | |
| IL-17 | TAGTCCACGTTCCCATCAGC | 121 |
| antisense strand | | |
| (A8-5 siRNA) | | |
| IL-17 | TTGTAGTAATCTGAGGACC | 122 |
| antisense strand | | |
| (A8-6 siRNA) | | |
| IL-6 | ACATGTCTCCTTTCTCAGGGC | 123 |
| antisense strand | | |
| (Bl-1 siRNA) | | |
| IL-6 | TTTCACCAGGCAAGTCTCCTC | 124 |
| antisense strand | | |
| (B1-2, B2, B15- | | |
| 1, B16-1, B17-1 | | |
| siRNA) | | |
| IL-6 | TACATTTGCCGAAGAGCCCTC | 125 |
| antisense strand | | |
| (B1-3 siRNA) | | |
| IL6R-alpha | ACTGTTCTGAAACTTCCTCAC | 126 |
| antisense strand | | |
| (B3-1, B4 | | |
| siRNA) | | |
| IL6R-alpha | TGTGAATGTCTTTGACCGTTC | 127 |
| antisense strand | | |
| (B3-2 siRNA) | | |
| IL6R-Beta | ATGATCTGATGTAACCTTCCC | 128 |
| antisense strand | | |
| (B3-3, B5 | | |
| siRNA) | | |
| ACE2 | TCATATAATGGCCTCAGCTGC | 129 |
| antisense strand | | |
| (B6-1, B7, B15- | | |
| 2, B16-2, B17-2 | | |
| siRNA) | | |
| ACE2 | TTCTGAACATTTCCTGGGTCC | 130 |
| antisense strand | | |
| (B6-2 siRNA) | | |
| ACE2 | TCTTGTTCTGGTCTTTCAGCC | 131 |
| antisense strand | | |
| (B6-3 siRNA) | | |
| SARS CoV- | CATCTTACCTTTCGGTCACAC | 132 |
| 2_ORF1ab | | |
| antisense strand | | |
| (B8-1, B14, | | |
| B18-1 siRNA) | | |
| SARS CoV- | GTCTGATCCCAATATTTAAA | 133 |
| 2_ORF1ab | | |
| antisense strand | | |
| (B12-1 siRNA) | | |
| SARS CoV- | GTGCGAGCTCTATTCTT | 134 |
| 2_ORF1ab | | |
| antisense strand | | |
| (B12-2, B13 | | |
| siRNA) | | |
| SARS CoV- | CCCTGTGATGAATCAACAGT | 135 |
| 2_ORF1ab | | |
| antisense strand | | |
| (B12-3 siRNA) | | |
| SARS CoV- | TAGGACAGAATAATCAGCAAC | 136 |
| 2_Spike Protein | | |
| antisense strand | | |
| (B11-1, B19-1 | | |
| siRNA) | | |
| SARS CoV- | TTTGTCTGACTTCATCACCTC | 137 |
| 2_Spike Protein | | |
| antisense strand | | |
| (B8-2, B9, B11- | | |
| 2, B18-2, B19-2 | | |
| siRNA) | | |
| SARS CoV- | ATTACAAGGTGTGCTACCGGC | 138 |
| 2_Spike Protein | | |
| antisense strand | | |
| (B11-3, B19-3 | | |
| siRNA) | | |
| SARS CoV- | ATTCAAGGCTCCCTCAGTTGC | 139 |
| 2_Nucleocapsid | | |
| Protein | | |
| antisense strand | | |
| (B8-3, B10, | | |
| B15-3, B16-3, | | |
| B17-3, B18-3 | | |
| siRNA) | | |
| ALK2 sense | GGCCTCATTATTCTCTCT | 140 |
| strand (A11-1 | | |
| siRNA) | | |
| ALK2 sense | GTGTTCGCAGTATGTCTT | 141 |
| strand (A11-2 | | |
| siRNA) | | |
| ALK2 sense | GCCTGCCTGCTGGGAGTT | 142 |
| strand (A11-3 | | |
| siRNA) | | |
| SOD1 sense | GAAGGAAAGTAATGGACCAGT | 143 |
| strand (A12-1, | | |
| A13-1 siRNA) | | |
| SOD1 sense | GGTCCTCACTTTAATCCTCTA | 144 |
| strand (A12-2, | | |
| A13-2 siRNA) | | |
| SOD1 sense | GGAGACTTGGGCAATGTGACT | 145 |
| strand (A12-3, | | |
| A13-3 siRNA) | | |
| ALK2 antisense | AGAGAGAATAATGAGGCC | 146 |
| strand (A11-1 | | |
| siRNA) | | |
| ALK2 antisense | AAGACATACTGCGAACAC | 147 |
| strand (A11-2 | | |
| siRNA) | | |
| ALK2 antisense | AACTCCCAGCAGGCAGGC | 148 |
| strand (A11-3 | | |
| siRNA) | | |
| SOD1 antisense | ACTGGTCCATTACTTTCCTTC | 149 |
| strand (A12-1, | | |
| A13-1 siRNA) | | |
| SOD1 antisense | TAGAGGATTAAAGTGAGGACC | 150 |
| strand (A12-2, | | |
| A13-2 siRNA) | | |
| SOD1 antisense | AGTCACATTGCCCAAGTCTCC | 151 |
| strand (A12-3, | | |
| A13-3 siRNA) | | |
| Compounds A9- | See Table 9 | 152-158 |
| A15 | | |
| Compounds B18 | See Table 10 | 159-166 |
| and A9-A15 | | |
| (plasmid | | |
| sequences) | | |
| IL-4 | ATCGTTAGCTTCTCCTGATAAACTAATTGCCTCACATTGTCACTGCAAAT | 167 |
| Human IL-4 | CGACACCTATTAATGGGTCTCACCTCCCAACTGCTTCCCCCTCTGTTCTT | |
| amino acid | CCTGCTAGCATGTGCCGGCAACTTTGTCCACGGACACAAGTGCGATATCA | |
| (Genbank | CCTTACAGGAGATCATCAAAACTTTGAACAGCCTCACAGAGCAGAAGACT | |
| NM_000589.4) | CTGTGCACCGAGTTGACCGTAACAGACATCTTTGCTGCCTCCAAGAACAC | |
| | AACTGAGAAGGAAACCTTCTGCAGGGCTGCGACTGTGCTCCGGCAGTTCT | |
| | ACAGCCACCATGAGAAGGACACTCGCTGCCTGGGTGCGACTGCACAGCAG | |
| | TTCCACAGGCACAAGCAGCTGATCCGATTCCTGAAACGGCTCGACAGGAA | |
| | CCTCTGGGGCCTGGCGGGCTTGAATTCCTGTCCTGTGAAGGAAGCCAACC | |
| | AGAGTACGTTGGAAAACTTCTTGGAAAGGCTAAAGACGATCATGAGAGAG | |
| | AAATATTCAAAGTGTTCGAGCTGAATATTTTAATTTATGAGTTTTTGATA | |
| | GCTTTATTTTTTAAGTATTTATATATTTATAACTCATCATAAAATAAAGT | |
| | ATATATAGAATCTAA | |
| IL-4 | MGLTSQLLPPLFFLLACAGNFVHGHKCDITLQEIIKTLNSLTEQKTLCTE | 168 |
| Human IL-4 | LTVTDIFAASKNTTEKETFCRAATVLRQFYSHHEKDTRCLGATAQQFHRH | |
| amino acid | KQLIRFLKRLDRNLWGLAGLNSCPVKEANQSTLENFLERLKTIMREKYSK | |
| (Genbank | CSS | |
| NP_000580.1) | | |
| Underlined: | | |
| signal sequence | | |
| Erythropoietin | CCTTTCCCAGATAGCACGCTCCGCCAGTCCCAAGGGTGCGCAACCGGCTG | 169 |
| (EPO) | CACTCCCCTCCCGCGACCCAGGGCCCGGGAGCAGCCCCCATGACCCACAC | |
| Human EPO | GCACGTCTGCAGCAGCCCCGCTCACGCCCCGGCGAGCCTCAACCCAGGCG | |
| amino acid | TCCTGCCCCTGCTCTGACCCCGGGTGGCCCCTACCCCTGGCGACCCCTCA | |
| (Genbank | CGCACACAGCCTCTCCCCCACCCCCACCCGCGCACGCACACATGCAGATA | |
| NM_000799.4) | ACAGCCCCGACCCCCGGCCAGAGCCGCAGAGTCCCTGGGCCACCCCGGCC | |
| | GCTCGCTGCGCTGCGCCGCACCGCGCTGTCCTCCCGGAGCCGGACCGGGG | |
| | CCACCGCGCCCGCTCTGCTCCGACACCGCGCCCCCTGGACAGCCGCCCTC | |
| | TCCTCCAGGCCCGTGGGGCTGGCCCTGCACCGCCGAGCTTCCCGGGATGA | |
| | GGGCCCCCGGTGTGGTCACCCGGCGCGCCCCAGGTCGCTGAGGGACCCCG | |
| | GCCAGGCGCGGAGATGGGGGTGCACGAATGTCCTGCCTGGCTGTGGCTTC | |
| | TCCTGTCCCTGCTGTCGCTCCCTCTGGGCCTCCCAGTCCTGGGCGCCCCA | |
| | CCACGCCTCATCTGTGACAGCCGAGTCCTGGAGAGGTACCTCTTGGAGGC | |
| | CAAGGAGGCCGAGAATATCACGACGGGCTGTGCTGAACACTGCAGCTTGA | |
| | ATGAGAATATCACTGTCCCAGACACCAAAGTTAATTTCTATGCCTGGAAG | |
| | AGGATGGAGGTCGGGCAGCAGGCCGTAGAAGTCTGGCAGGGCCTGGCCCT | |
| | GCTGTCGGAAGCTGTCCTGCGGGGCCAGGCCCTGTTGGTCAACTCTTCCC | |
| | AGCCGTGGGAGCCCCTGCAGCTGCATGTGGATAAAGCCGTCAGTGGCCTT | |
| | CGCAGCCTCACCACTCTGCTTCGGGCTCTGGGAGCCCAGAAGGAAGCCAT | |
| | CTCCCCTCCAGATGCGGCCTCAGCTGCTCCACTCCGAACAATCACTGCTG | |
| | ACACTTTCCGCAAACTCTTCCGAGTCTACTCCAATTTCCTCCGGGGAAAG | |
| | CTGAAGCTGTACACAGGGGAGGCCTGCAGGACAGGGGACAGATGACCAGG | |
| | TGTGTCCACCTGGGCATATCCACCACCTCCCTCACCAACATTGCTTGTGC | |
| | CACACCCTCCCCCGCCACTCCTGAACCCCGTCGAGGGGCTCTCAGCTCAG | |
| | CGCCAGCCTGTCCCATGGACACTCCAGTGCCAGCAATGACATCTCAGGGG | |
| | CCAGAGGAACTGTCCAGAGAGCAACTCTGAGATCTAAGGATGTCACAGGG | |
| | CCAACTTGAGGGCCCAGAGCAGGAAGCATTCAGAGAGCAGCTTTAAACTC | |
| | AGGGACAGAGCCATGCTGGGAAGACGCCTGAGCTCACTCGGCACCCTGCA | |
| | AAATTTGATGCCAGGACACGCTTTGGAGGCGATTTACCTGTTTTCGCACC | |
| | TACCATCAGGGACAGGATGACCTGGATAACTTAGGTGGCAAGCTGTGACT | |
| | TCTCCAGGTCTCACGGGCATGGGCACTCCCTTGGTGGCAAGAGCCCCCTT | |
| | GACACCGGGGTGGTGGGAACCATGAAGACAGGATGGGGGCTGGCCTCTGG | |
| | CTCTCATGGGGTCCAAGTTTTGTGTATTCTTCAACCTCATTGACAAGAAC | |
| | TGAAACCACCAA | |
| Erythropoietin | MGVHECPAWLWLLLSLLSLPLGLPVLGAPPRLICDSRVLERYLLEAKEAE | 170 |
| (EPO) | NITTGCAEHCSLNENITVPDTKVNFYAWKRMEVGQQAVEVWQGLALLSEA | |
| Human EPO | VLRGQALLVNSSQPWEPLQLHVDKAVSGLRSLTTLLRALGAQKEAISPPD | |
| amino acid | AASAAPLRTITADTFRKLFRVYSNFLRGKLKLYTGEACRTGDR | |
| (Genbank | | |
| NP_000790.2) | | |
| Underlined: | | |
| signal sequence | | |
| ALK2 mRNA | GAGGTGGAGTATGGCACTATCG | 171 |
| forward primer | | |
| ALK2 mRNA | CACTCCAACAGTGTAATCTGGCG | 172 |
| reverse primer | | |
| Human 18S | ACCCGTTGAACCCCATTCGTGA | 173 |
| rRNA forward | | |
| primer | | |
| Human 18S | GCCTCACTAAACCATCCAATCGG | 174 |
| rRNA reverse | | |
| primer | | |
| Human SOD1 | CTCACTCTCAGGAGACCATTGC | 175 |
| mRNA forward | | |
| primer | | |
| Human SOD1 | CCACAAGCCAAACGACTTCCAG | 176 |
| mRNA reverse | | |
| primer | | |
| Compound A1 | AUAGUGAGUCGUAUUAACGUACCAACAACAAGGAAGUGCUAAAGAAACUU | 177 |
| RNA sequence | GUUCUUUAGCACUUCCUUGUUUAUCUUAGAGGCAUAUGCCUGCCACCAUG | |
| | ACCAUCCUGUUUCUGACAAUGGUCAUCAGCUACUUCGGCUGCAUGAAGGC | |
| | CGUGAAGAUGCACACCAUGAGCAGCAGCCACCUGUUCUAUCUGGCCCUGU | |
| | GCCUGCUGACCUUUACCAGCUCUGCUACCGCCGGACCUGAGACACUUUGU | |
| | GGCGCUGAACUGGUGGACGCCCUGCAGUUUGUGUGUGGCGACAGAGGCUU | |
| | CUACUUCAACAAGCCCACAGGCUACGGCAGCAGCUCUAGAAGGGCUCCUC | |
| | AGACCGGAAUCGUGGACGAGUGCUGCUUCAGAAGCUGCGACCUGCGGCGG | |
| | CUGGAAAUGUAUUGUGCCCCUCUGAAGCCUGCCAAGAGCGCCUAAUUUAU | |
| | CUUAGAGGCAUAUCCCU | |
| | (all Us are modified; N¹-methylpseudouridine) | |
| Compound A2 | AUAGUGAGUCGUAUUAACGUACCAACAACAAGGAGUGCUAAAGAAACUUG | 178 |
| RNA sequence | UUCUUUAGCACUCCUUGUUUAUCUUAGAGGCAUAUCCCUGCCACCAUGAC | |
| | CAUCCUGUUUCUGACAAUGGUCAUCAGCUACUUCGGCUGCAUGAAGGCCG | |
| | UGAAGAUGCACACCAUGAGCAGCAGCCACCUGUUCUAUCUGGCCCUGUGC | |
| | CUGCUGACCUUUACCAGCUCUGCUACCGCCGGACCUGAGACACUUUGUGG | |
| | CGCUGAACUGGUGGACGCCCUGCAGUUUGUGUGUGGCGACAGAGGCUUCU | |
| | ACUUCAACAAGCCCACAGGCUACGGCAGCAGCUCUAGAAGGGCUCCUCAG | |
| | ACCGGAAUCGUGGACGAGUGCUGCUUCAGAAGCUGCGACCUGCGGCGGCU | |
| | GGAAAUGUAUUGUGCCCCUCUGAAGCCUGCCAAGAGCGCCUAAUUUAUCU | |
| | UAGAGGCAUAUCCCU | |
| | (all Us are modified; N¹-methylpseudouridine) | |
| Compound A3 | AUAGUGAGUCGUAUUAACGUACCAACAACAAGGAGUGCUAAAGAAACUUG | 179 |
| RNA sequence | UUCUUUAGCACUCCUUGUUUAUCUUAGAGGCAUAUCCCUACGUACCAACA | |
| | AGAGAGUGAUUGAGAGUGGACUUGCCACUCUCAAUCACUCUCUUUAUCUU | |
| | AGAGGCAUAUCCCUACGUACCAACAAGAGAGCUCUGUCUGGACCACUUGG | |
| | GUCCAGACAGAGCUCUCUUUAUCUUAGAGGCAUAUCCCUGCCACCAUGAC | |
| | CAUCCUGUUUCUGACAAUGGUCAUCAGCUACUUCGGCUGCAUGAAGGCCG | |
| | UGAAGAUGCACACCAUGAGCAGCAGCCACCUGUUCUAUCUGGCCCUGUGC | |
| | CUGCUGACCUUUACCAGCUCUGCUACCGCCGGACCUGAGACACUUUGUGG | |
| | CGCUGAACUGGUGGACGCCCUGCAGUUUGUGUGUGGCGACAGAGGCUUCU | |
| | ACUUCAACAAGCCCACAGGCUACGGCAGCAGCUCUAGAAGGGCUCCUCAG | |
| | ACCGGAAUCGUGGACGAGUGCUGCUUCAGAAGCUGCGACCUGCGGCGGCU | |
| | GGAAAUGUAUUGUGCCCCUCUGAAGCCUGCCAAGAGCGCCUAAUUUAUCU | |
| | UAGAGGCAUAUCCCU | |
| | (all Us are modified; N¹-methylpseudouridine) | |
| Compound A6 | AUAGUGAGUCGUAUUAACGUACCAACAAGAAAGAUGAUAAGCCCACUCUA | 180 |
| RNA sequence | CUUGAGAGUGGGCUUAUCAUCUUUCUUUAUCUUAGAGGCAUAUCCCUGCC | |
| | ACCAUGACCAUCCUGUUUCUGACAAUGGUCAUCAGCUACUUCGGCUGCAU | |
| | GAAGGCCGUGAAGAUGCACACCAUGAGCAGCAGCCACCUGUUCUAUCUGG | |
| | CCCUGUGCCUGCUGACCUUUACCAGCUCUGCUACCGCCGGACCUGAGACA | |
| | CUUUGUGGCGCUGAACUGGUGGACGCCCUGCAGUUUGUGUGUGGCGACAG | |
| | AGGCUUCUACUUCAACAAGCCCACAGGCUACGGCAGCAGCUCUAGAAGGG | |
| | CUCCUCAGACCGGAAUCGUGGACGAGUGCUGCUUCAGAAGCUGCGACCUG | |
| | CGGCGGCUGGAAAUGUAUUGUGCCCCUCUGAAGCCUGCCAAGAGCGCCUA | |
| | AUUUAUCUUAGAGGCAUAUCCCU | |
| | (all Us are modified; N¹-methylpseudouridine) | |
| Compound A7 | AUAGUGAGUCGUAUUAACGUACCAACAAGAAAGAUGAUAAGCCCACUCUA | 181 |
| RNA sequence | CUUGAGAGUGGGCUUAUCAUCUUUCUUUAUCUUAGAGGCAUAUCCCUACG | |
| | UACCAACAAGGUGAUGUCUGGUCCAUAUGAACUUGUCAUAUGGACCAGAC | |
| | AUCACCUUUAUCUUAGAGGCAUAUCCCUACGUACCAACAAGAUGAUAAGC | |
| | CCACUCUAACUUGUAGAGUGGGCUUAUCAUCUUUAUCUUAGAGGCAUAUC | |
| | CCUGCCACCAUGACCAUCCUGUUUCUGACAAUGGUCAUCAGCUACUUCGG | |
| | CUGCAUGAAGGCCGUGAAGAUGCACACCAUGAGCAGCAGCCACCUGUUCU | |
| | AUCUGGCCCUGUGCCUGCUGACCUUUACCAGCUCUGCUACCGCCGGACCU | |
| | GAGACACUUUGUGGCGCUGAACUGGUGGACGCCCUGCAGUUUGUGUGUGG | |
| | CGACAGAGGCUUCUACUUCAACAAGCCCACAGGCUACGGCAGCAGCUCUA | |
| | GAAGGGCUCCUCAGACCGGAAUCGUGGACGAGUGCUGCUUCAGAAGCUGC | |
| | GACCUGCGGCGGCUGGAAAUGUAUUGUGCCCCUCUGAAGCCUGCCAAGAG | |
| | CGCCUAAUUUAUCUUAGAGGCAUAUCCCU | |
| | (all Us are modified; N¹-methylpseudouridine) | |
| Compound A8 | AUAGUGAGUCGUAUUAACGUACCAACAAGGCGUGGAGCUGAGAGAUAAAC | 182 |
| RNA sequence | UUGUUAUCUCUCAGCUCCACGCCUUUAUCUUAGAGGCAUAUCCCUACGUA | |
| | CCAACAAGGGCCUGUACCUCAUCUACUACUUGAGUAGAUGAGGUACAGGC | |
| | CCUUUAUCUUAGAGGCAUAUCCCUACGUACCAACAAGGUAUGAGCCCAUC | |
| | UAUCUACUUGAGAUAGAUGGGCUCAUACCUUUAUCUUAGAGGCAUAUCCC | |
| | UACGUACCAACAAGCAAUGAGGACCCUGAGAGAUACUUGAUCUCUCAGGG | |
| | UCCUCAUUGCUUUAUCUUAGAGGCAUAUCCCUACGUACCAACAAGCUGAU | |
| | GGGAACGUGGACUAACUUGUAGUCCACGUUCCCAUCAGCUUUAUCUUAGA | |
| | GGCAUAUCCCUACGUACCAACAAGGUCCUCAGAUUACUACAAACUUGUUG | |
| | UAGUAAUCUGAGGACCUUUAUCUUAGAGGCAUAUCCCUGCCACCAUGGGA | |
| | CUGACAUCUCAACUGCUGCCUCCACUGUUCUUUCUGCUGGCCUGCGCCGG | |
| | CAAUUUUGUGCACGGCCACAAGUGCGACAUCACCCUGCAAGAGAUCAUCA | |
| | AGACCCUGAACAGCCUGACCGAGCAGAAAACCCUGUGCACCGAGCUGACC | |
| | GUGACCGAUAUCUUUGCCGCCAGCAAGAACACAACCGAGAAAGAGACAUU | |
| | CUGCAGAGCCGCCACCGUGCUGAGACAGUUCUACAGCCACCACGAGAAGG | |
| | ACACCAGAUGCCUGGGAGCUACAGCCCAGCAGUUCCACAGACACAAGCAG | |
| | CUGAUCCGGUUCCUGAAGCGGCUGGACAGAAAUCUGUGGGGACUCGCCGG | |
| | CCUGAAUAGCUGCCCUGUGAAAGAGGCCAACCAGUCUACCCUGGAAAACU | |
| | UCCUGGAACGGCUGAAAACCAUCAUGCGCGAGAAGUACAGCAAGUGCAGC | |
| | AGCUGAUUUAUCUUAGAGGCAUAUCCCU | |
| | (all Us are modified; N¹-methylpseudouridine) | |
| Compound A9 | AUAGUGAGUCGUAUUAACGUACCAACAAGGCGUGGAGCUGAGAGAUAAAC | 183 |
| RNA sequence | UUGUUAUCUCUCAGCUCCACGCCUUUAUCUUAGAGGCAUAUCCCUACGUA | |
| | CCAACAAGGGCCUGUACCUCAUCUACUACUUGAGUAGAUGAGGUACAGGC | |
| | CCUUUAUCUUAGAGGCAUAUCCCUACGUACCAACAAGGUAUGAGCCCAUC | |
| | UAUCUACUUGAGAUAGAUGGGCUCAUACCUUUAUCUUAGAGGCAUAUCCC | |
| | UGCCACCAUGGGACUGACAUCUCAACUGCUGCCUCCACUGUUCUUUCUGC | |
| | UGGCCUGCGCCGGCAAUUUUGUGCACGGCCACAAGUGCGACAUCACCCUG | |
| | CAAGAGAUCAUCAAGACCCUGAACAGCCUGACCGAGCAGAAAACCCUGUG | |
| | CACCGAGCUGACCGUGACCGAUAUCUUUGCCGCCAGCAAGAACACAACCG | |
| | AGAAAGAGACAUUCUGCAGAGCCGCCACCGUGCUGAGACAGUUCUACAGC | |
| | CACCACGAGAAGGACACCAGAUGCCUGGGAGCUACAGCCCAGCAGUUCCA | |
| | CAGACACAAGCAGCUGAUCCGGUUCCUGAAGCGGCUGGACAGAAAUCUGU | |
| | GGGGACUCGCCGGCCUGAAUAGCUGCCCUGUGAAAGAGGCCAACCAGUCU | |
| | ACCCUGGAAAACUUCCUGGAACGGCUGAAAACCAUCAUGCGCGAGAAGUA | |
| | CAGCAAGUGCAGCAGCUGAUUUAUCUUAGAGGCAUAUCCCU | |
| | (all Us are modified; N¹-methylpseudouridine) | |
| Compound A10 | GCCACCAUGGGACUGACAUCUCAACUGCUGCCUCCACUGUUCUUUCUGCU | 184 |
| RNA sequence | GGCCUGCGCCGGCAAUUUUGUGCACGGCCACAAGUGCGACAUCACCCUGC | |
| | AAGAGAUCAUCAAGACCCUGAACAGCCUGACCGAGCAGAAAACCCUGUGC | |
| | ACCGAGCUGACCGUGACCGAUAUCUUUGCCGCCAGCAAGAACACAACCGA | |
| | GAAAGAGACAUUCUGCAGAGCCGCCACCGUGCUGAGACAGUUCUACAGCC | |
| | ACCACGAGAAGGACACCAGAUGCCUGGGAGCUACAGCCCAGCAGUUCCAC | |
| | AGACACAAGCAGCUGAUCCGGUUCCUGAAGCGGCUGGACAGAAAUCUGUG | |
| | GGGACUCGCCGGCCUGAAUAGCUGCCCUGUGAAAGAGGCCAACCAGUCUA | |
| | CCCUGGAAAACUUCCUGGAACGGCUGAAAACCAUCAUGCGCGAGAAGUAC | |
| | AGCAAGUGCAGCAGCUGAAUAGUGAGUCGUAUUAACGUACCAACAAGGCG | |
| | UGGAGCUGAGAGAUAAACUUGUUAUCUCUCAGCUCCACGCCUUUAUCUUA | |
| | GAGGCAUAUCCCUACGUACCAACAAGGGCCUGUACCUCAUCUACUACUUG | |
| | AGUAGAUGAGGUACAGGCCCUUUAUCUUAGAGGCAUAUCCCUACGUACCA | |
| | ACAAGGUAUGAGCCCAUCUAUCUACUUGAGAUAGAUGGGCUCAUACCUUU | |
| | AUCUUAGAGGCAUAUCCCUUUUAUCUUAGAGGCAUAUCCCU | |
| | (all Us are modified; N¹-methylpseudouridine) | |
| Compound A11 | GCCACCAUGACCAUCCUGUUUCUGACAAUGGUCAUCAGCUACUUCGGCUG | 185 |
| RNA sequence | CAUGAAGGCCGUGAAGAUGCACACCAUGAGCAGCAGCCACCUGUUCUAUC | |
| | UGGCCCUGUGCCUGCUGACCUUUACCAGCUCUGCUACCGCCGGACCUGAG | |
| | ACACUUUGUGGCGCUGAACUGGUGGACGCCCUGCAGUUUGUGUGUGGCGA | |
| | CAGAGGCUUCUACUUCAACAAGCCCACAGGCUACGGCAGCAGCUCUAGAA | |
| | GGGCUCCUCAGACCGGAAUCGUGGACGAGUGCUGCUUCAGAAGCUGCGAC | |
| | CUGCGGCGGCUGGAAAUGUAUUGUGCCCCUCUGAAGCCUGCCAAGAGCGC | |
| | CUAAAUAGUGAGUCGUAUUAACGUACCAACAAGGCCUCAUUAUUCUCUCU | |
| | ACUUGAGAGAGAAUAAUGAGGCCUUUAUCUUAGAGGCAUAUCCCUACGUA | |
| | CCAACAAGUGUUCGCAGUAUGUCUUACUUGAAGACAUACUGCGAACACUU | |
| | UAUCUUAGAGGCAUAUCCCUACGUACCAACAAGCCUGCCUGCUGGGAGUU | |
| | ACUUGAACUCCCAGCAGGCAGGCUUUAUCUUAGAGGCAUAUCCCUUUUAU | |
| | CUUAGAGGCAUAUCCCU | |
| | (all Us are modified; N¹-methylpseudouridine) | |
| Compound A12 | AUAGUGAGUCGUAUUAACGUACCAACAAGAAGGAAAGUAAUGGACCAGUA | 186 |
| RNA sequence | CUUGACUGGUCCAUUACUUUCCUUCUUUAUCUUAGAGGCAUAUCCCUACG | |
| | UACCAACAAGGUCCUCACUUUAAUCCUCUAACUUGUAGAGGAUUAAAGUG | |
| | AGGACCUUUAUCUUAGAGGCAUAUCCCUACGUACCAACAAGGAGACUUGG | |
| | GCAAUGUGACUACUUGAGUCACAUUGCCCAAGUCUCCUUUAUCUUAGAGG | |
| | CAUAUCCCUGCCACCAUGGGCAAGAUUAGCAGCCUGCCUACACAGCUGUU | |
| | CAAGUGCUGCUUCUGCGACUUCCUGAAAGUGAAGAUGCACACCAUGAGCA | |
| | GCAGCCACCUGUUCUAUCUGGCCCUGUGCCUGCUGACCUUUACCAGCUCU | |
| | GCUACCGCCGGACCUGAGACACUUUGUGGCGCUGAACUGGUGGACGCCCU | |
| | GCAGUUUGUGUGUGGCGACAGAGGCUUCUACUUCAACAAGCCCACAGGCU | |
| | ACGGCAGCAGCUCUAGAAGGGCUCCUCAGACCGGAAUCGUGGACGAGUGC | |
| | UGUUUCAGAAGCUGCGACCUGCGGCGGCUGGAAAUGUAUUGUGCCCCUCU | |
| | GAAGCCUGCCAAGAGCGCCUAAUUUAUCUUAGAGGCAUAUCCCU | |
| | (all Us are modified; N¹-methylpseudouridine) | |
| Compound A13 | AUAGUGAGUCGUAUUAACGUACCAACAAGAAGGAAAGUAAUGGACCAGUA | 187 |
| RNA sequence | CUUGACUGGUCCAUUACUUUCCUUCUUUAUCUUAGAGGCAUAUCCCUACG | |
| | UACCAACAAGGUCCUCACUUUAAUCCUCUAACUUGUAGAGGAUUAAAGUG | |
| | AGGACCUUUAUCUUAGAGGCAUAUCCCUACGUACCAACAAGGAGACUUGG | |
| | GCAAUGUGACUACUUGAGUCACAUUGCCCAAGUCUCCUUUAUCUUAGAGG | |
| | CAUAUCCCUGCCACCAUGGGAGUGCAUGAAUGUCCUGCUUGGCUGUGGCU | |
| | GCUGCUGAGCCUGCUGUCUCUGCCUCUGGGACUGCCUGUUCUUGGAGCCC | |
| | CUCCUAGACUGAUCUGCGACAGCAGAGUGCUGGAAAGAUACCUGCUGGAA | |
| | GCCAAAGAGGCCGAGAACAUCACCACAGGCUGUGCCGAGCACUGCAGCCU | |
| | GAACGAGAAUAUCACCGUGCCUGACACCAAAGUGAACUUCUACGCCUGGA | |
| | AGCGGAUGGAAGUGGGCCAGCAGGCUGUGGAAGUUUGGCAAGGACUGGCC | |
| | CUGCUGAGCGAAGCUGUUCUGAGAGGACAGGCUCUGCUGGUCAACAGCUC | |
| | UCAGCCUUGGGAACCUCUGCAACUGCACGUGGACAAGGCCGUGUCUGGCC | |
| | UGAGAAGCCUGACCACACUGCUGAGAGCACUGGGAGCCCAGAAAGAGGCC | |
| | AUCUCUCCACCUGAUGCUGCCUCUGCUGCCCCUCUGAGAACCAUCACCGC | |
| | CGACACCUUCAGAAAGCUGUUCCGGGUGUACAGCAACUUCCUGCGGGGCA | |
| | AGCUGAAGCUGUACACAGGCGAGGCUUGCAGAACCGGCGACAGAUAAUUU | |
| | AUCUUAGAGGCAUAUCCCU | |
| | (all Us are modified; N¹-methylpseudouridine) | |
| Compound A14 | AUAGUGAGUCGUAUUAACGUACCAACAAGAAAGAUGAUAAGCCCACUCUA | 188 |
| RNA sequence | CUUGAGAGUGGGCUUAUCAUCUUUCUUUAUCUUAGAGGCAUAUCCCUACG | |
| | UACCAACAAGGUGAUGUCUGGUCCAUAUGAACUUGUCAUAUGGACCAGAC | |
| | AUCACCUUUAUCUUAGAGGCAUAUCCCUACGUACCAACAAGAUGAUAAGC | |
| | CCACUCUAACUUGUAGAGUGGGCUUAUCAUCUUUAUCUUAGAGGCAUAUC | |
| | CCUGCCACCAUGGGCAAGAUUAGCAGCCUGCCUACACAGCUGUUCAAGUG | |
| | CUGCUUCUGCGACUUCCUGAAAGUGAAGAUGCACACCAUGAGCAGCAGCC | |
| | ACCUGUUCUAUCUGGCCCUGUGCCUGCUGACCUUUACCAGCUCUGCUACC | |
| | GCCGGACCUGAGACACUUUGUGGCGCUGAACUGGUGGACGCCCUGCAGUU | |
| | UGUGUGUGGCGACAGAGGCUUCUACUUCAACAAGCCCACAGGCUACGGCA | |
| | GCAGCUCUAGAAGGGCUCCUCAGACCGGAAUCGUGGACGAGUGCUGUUUC | |
| | AGAAGCUGCGACCUGCGGCGGCUGGAAAUGUAUUGUGCCCCUCUGAAGCC | |
| | UGGCAAGAGCGCCUAAUUUAUCUUAGAGGCAUAUCCCU | |
| | (all Us are modified; N¹-methylpseudouridine) | |
| Compound A15 | GCCACCAUGGGCAAGAUUAGCAGCCUGCCUACACAGCUGUUCAAGUGCUG | 189 |
| RNA sequence | CUUCUGCGACUUCCUGAAAGUGAAGAUGCACACCAUGAGCAGCAGCCACC | |
| | UGUUCUAUCUGGCCCUGUGCCUGCUGACCUUUACCAGCUCUGCUACCGCC | |
| | GGACCUGAGACACUUUGUGGCGCUGAACUGGUGGACGCCCUGCAGUUUGU | |
| | GUGUGGCGACAGAGGCUUCUACUUCAACAAGCCCACAGGCUACGGCAGCA | |
| | GCUCUAGAAGGGCUCCUCAGACCGGAAUCGUGGACGAGUGCUGUUUCAGA | |
| | AGCUGCGACCUGCGGCGGCUGGAAAUGUAUUGUGCCCCUCUGAAGCCUGC | |
| | CAAGAGCGCCUAAAUAGUGAGUCGUAUUAACGUACCAACAAGAAAGAUGA | |
| | UAAGCCCACUCUACUUGAGAGUGGGCUUAUCAUCUUUCUUUAUCUUAGAG | |
| | GCAUAUCCCUACGUACCAACAAGGUGAUGUCUGGUCCAUAUGAACUUGUC | |
| | AUAUGGACCAGACAUCACCUUUAUCUUAGAGGCAUAUCCCUACGUACCAA | |
| | CAAGAUGAUAAGCCCACUCUAACUUGUAGAGUGGGCUUAUCAUCUUUAUC | |
| | UUAGAGGCAUAUCCCUUUUAUCUUAGAGGCAUAUCCCU | |
| | (all Us are modified; N¹-methylpseudouridine) | |
| Compound B18 | GCCACCAUGUCUAGCAGCUCUUGGCUGCUGCUGUCUCUGGUGGCUGUGAC | 190 |
| RNA sequence | AGCCGCUCAGAGCACCAUUGAGGAACAGGCCAAGACCUUCCUGGACAAGU | |
| | UCAACCACGAGGCCGAGGACCUGUUCUACCAGUCUAGCCUGGCCAGCUGG | |
| | AACUACAACACCAACAUCACCGAAGAGAACGUGCAGAACAUGAACAACGC | |
| | CGGCGACAAGUGGAGCGCCUUCCUGAAAGAGCAGAGCACACUGGCCCAGA | |
| | UGUACCCUCUGCAAGAGAUCCAGAACCUGACCGUGAAGCUCCAGCUGCAG | |
| | GCCCUCCAGCAGAAUGGAAGCUCUGUGCUGAGCGAGGACAAGAGCAAGCG | |
| | GCUGAACACCAUCCUGAAUACCAUGAGCACCAUCUACAGCACCGGCAAAG | |
| | UGUGCAACCCCGACAAUCCCCAAGAGUGCCUGCUGCUGGAACCCGGCCUG | |
| | AAUGAGAUCAUGGCCAACAGCCUGGACUACAACGAGAGACUGUGGGCCUG | |
| | GGAGUCUUGGAGAAGCGAAGUGGGAAAGCAGCUGCGGCCCCUGUACGAGG | |
| | AAUACGUGGUGCUGAAGAACGAGAUGGCCAGAGCCAACCACUACGAGGAC | |
| | UACGGCGACUAUUGGAGAGGCGACUACGAAGUGAAUGGCGUGGACGGCUA | |
| | CGACUACAGCAGAGGCCAGCUGAUCGAGGACGUGGAACACACCUUCGAGG | |
| | AAAUCAAGCCUCUGUACGAGCAUCUGCACGCCUACGUGCGGGCCAAGCUG | |
| | AUGAAUGCUUACCCCAGCUACAUCAGCCCCAUCGGCUGUCUGCCUGCUCA | |
| | UCUGCUGGGAGACAUGUGGGGCAGAUUCUGGACCAACCUGUACAGCCUGA | |
| | CAGUGCCCUUCGGCCAGAAACCUAACAUCGACGUGACCGACGCCAUGGUG | |
| | GAUCAGGCUUGGGAUGCCCAGCGGAUCUUCAAAGAGGCCGAGAAGUUCUU | |
| | CGUGUCCGUGGGCCUGCCUAAUAUGACCCAAGGCUUCUGGGAGAACUCCA | |
| | UGCUGACAGACCCCGGCAAUGUGCAGAAAGCCGUGUGUCAUCCUACCGCC | |
| | UGGGAUCUCGGCAAGGGCGACUUCAGAAUCCUGAUGUGCACCAAAGUGAC | |
| | GAUGGACGACUUCCUGACAGCCCACCACGAGAUGGGCCACAUCCAGUACG | |
| | AUAUGGCCUACGCCGCUCAGCCCUUCCUGCUGAGAAAUGGCGCCAAUGAG | |
| | GGCUUCCACGAAGCCGUGGGAGAGAUCAUGAGCCUGUCUGCCGCCACACC | |
| | UAAGCACCUGAAGUCUAUCGGACUGCUGAGCCCCGACUUCCAAGAGGACA | |
| | ACGAGACAGAGAUCAACUUCCUGCUCAAGCAGGCCCUGACCAUCGUGGGC | |
| | ACACUGCCCUUUACCUACAUGCUGGAAAAGUGGCGGUGGAUGGUCUUUAA | |
| | GGGCGAGAUCCCCAAGGACCAGUGGAUGAAGAAAUGGUGGGAGAUGAAGC | |
| | GCGAGAUCGUGGGCGUUGUGGAACCUGUGCCUCACGACGAGACAUACUGC | |
| | GAUCCUGCCAGCCUGUUUCACGUGUCCAACGACUACUCCUUCAUCCGGUA | |
| | CUACACCCGGACACUGUACCAGUUCCAGUUUCAAGAGGCUCUGUGCCAGG | |
| | CCGCCAAGCACGAAGGACCUCUGCACAAGUGCGACAUCAGCAACUCUACA | |
| | GAGGCCGGACAGAAACUGUUCAACAUGCUGCGGCUGGGCAAGAGCGAGCC | |
| | UUGGACACUGGCUCUGGAAAAUGUCGUGGGCGCCAAGAAUAUGAACGUGC | |
| | GGCCACUGCUGAACUACUUCGAGCCCCUGUUCACCUGGCUGAAGGACCAG | |
| | AACAAGAACAGCUUCGUCGGCUGGUCCACCGAUUGGAGCCCUUACGCCGA | |
| | CCAGAGCAUCAAAGUGCGGAUCAGCCUGAAAAGCGCCCUGGGCGAUAAGG | |
| | CCUAUGAGUGGAACGACAAUGAGAUGUACCUGUUCCGGUCCAGCGUGGCC | |
| | UAUGCUAUGCGGCAGUACUUUCUGAAAGUCAAGAACCAGAUGAUCCUGUU | |
| | CGGCGAAGAGGAUGUGCGCGUGGCCAACCUGAAGCCUCGGAUCAGCUUCA | |
| | ACUUCUUCGUGACUGCCCCUAAGAACGUGUCCGACAUCAUCCCCAGAACC | |
| | GAGGUGGAAAAGGCCAUCAGAAUGAGCAGAAGCCGGAUCAACGACGCCUU | |
| | CCGGCUGAACGACAACUCCCUGGAAUUCCUGGGCAUUCAGCCCACACUGG | |
| | GCCCUCCAAAUCAGCCUCCUGUGUCCUAAAUAGUGAGUCGUAUUAACGUA | |
| | CCAACAAGUGUGACCGAAAGGUAAGAUGACUUGCAUCUUACCUUUCGGUC | |
| | ACACUUUAUCUUAGAGGCAUAUCCCUACGUACCAACAAGAGGUGAUGAAG | |
| | UCAGACAAAACUUGUUUGUCUGACUUCAUCACCUCUUUAUCUUAGAGGCA | |
| | UAUCCCUACGUACCAACAAGCAACUGAGGGAGCCUUGAAUACUUGAUUCA | |
| | AGGCUCCCUCAGUUGCUUUAUCUUAGAGGCAUAUCCCUUUUAUCUUAGAG | |
| | GCAUAUCCCU | |
| | (all Us are modified; N¹-methylpseudouridine) | |

1.-77. (canceled)

78. A composition comprising a recombinant RNA construct or a vector encoding the recombinant RNA construct, wherein the recombinant RNA construct comprises:
(i) at least one RNA sequence comprising a small interfering RNA (siRNA) capable of binding to a target RNA; and
(ii) at least one RNA sequence comprising a messenger RNA (mRNA) encoding a protein of interest,
wherein the at least one RNA sequence comprising the mRNA is located upstream or downstream of the at least one RNA sequence comprising the siRNA; and
wherein the target RNA is different from the mRNA encoding the protein of interest.

79. The composition of claim 78, wherein the at least one RNA sequence comprising the siRNA is downstream of the at least one RNA sequence comprising the mRNA.

80. The composition of claim 78, wherein the at least one RNA sequence comprising the siRNA is upstream of the at least one RNA sequence comprising the mRNA.

81. The composition of claim 78, wherein the composition further comprises a cell, wherein the cell comprises the recombinant RNA construct or the vector encoding the recombinant RNA construct, and wherein the siRNA does not inhibit expression of the protein of interest in the cell.

82. The composition of claim 78, wherein the composition further comprises a cell, wherein the cell comprises the recombinant RNA construct or the vector encoding the recombinant RNA construct, and wherein
(a) an expression level of the protein of interest is higher in the cell compared to the expression level of the protein of interest in a corresponding cell without the recombinant RNA construct or the vector encoding the recombinant RNA construct; and/or
(b) an expression level of the target RNA is lower in the cell compared to the expression level of the target RNA in a corresponding cell without the recombinant RNA construct or the vector encoding the recombinant RNA construct.

83. The composition of claim 79, wherein the composition further comprises a cell, wherein the cell comprises the recombinant RNA construct or the vector encoding the recombinant RNA construct, and wherein
(a) an expression level of the protein of interest is higher in the cell compared to the expression level of the protein of interest in a corresponding cell comprising a corresponding recombinant RNA construct or a vector encoding the corresponding recombinant RNA construct in which the at least one RNA sequence comprising the siRNA is upstream of the at least one RNA sequence comprising the mRNA; and/or
(b) an expression level of the target RNA is lower in the cell compared to the expression level of the target RNA in a corresponding cell comprising a corresponding recombinant RNA construct or a vector encoding the corresponding recombinant RNA construct in which the at least one RNA sequence comprising the siRNA is upstream of the at least one RNA sequence comprising the mRNA.

84. The composition of claim 78, wherein the recombinant RNA construct further comprises a linker; wherein the linker comprises a nucleic acid sequence that connects the at least one RNA sequence comprising the siRNA and the at least one RNA sequence comprising the mRNA.

85. The composition of claim 78, wherein the recombinant RNA construct comprises two or more RNA sequences comprising an siRNA, wherein each of the two or more RNA sequences comprises an siRNA capable of binding to a same target RNA or a different target RNA.

86. The composition of claim 85, wherein the recombinant RNA construct further comprises a linker; wherein the linker comprises a nucleic acid sequence that connects each of the two or more nucleic acid sequences comprising the siRNA.

87. The composition of claim 86, wherein the linker comprises a tRNA linker, a 2A peptide linker or a flexible linker.

88. The composition of claim 78, wherein the recombinant RNA construct comprises two or more RNA sequences comprising an mRNA, wherein each of the two or more RNA sequences comprises an mRNA encodes a same protein of interest or a different protein of interest.

89. The composition of claim 88, wherein the recombinant RNA construct further comprises a linker; wherein the linker comprises a nucleic acid sequence that connects each of the two or more nucleic acid sequences comprising the mRNA.

90. The composition of claim 78, wherein the target RNA is an mRNA encoding a protein selected from the group consisting of Interleukin 8 (IL-8), Interleukin 1 beta (IL-1 beta), Interleukin 17 (IL-17), Tumor Necrosis Factor alpha (TNF-alpha), Activin Receptor-like Kinase 2 (ALK2), and Superoxide Dismutase 1 (SOD1).

91. The composition of claim 78, wherein the protein of interest is selected from the group consisting of Insulin-like Growth Factor 1 (IGF-1), Interleukin 4 (IL-4), and Erythropoietin (EPO).

92. The composition of claim 78, wherein (a) the recombinant RNA construct is encoded by a sequence selected from the group consisting of SEQ ID NOs: 1-3, 6-8, 9-11, 14-16, 152-158, and 160-166, or (b) the recombinant RNA construct comprises a sequence selected from the group consisting of SEQ ID NOs: 177-189.

93. The composition of claim 78, wherein the siRNA comprises a sense strand sequence encoded by a sequence selected from the group consisting of SEQ ID NOs: 80-92 and SEQ ID NOs: 140-145.

94. The composition of claim 79, wherein the composition further comprises a cell, wherein the cell comprises the recombinant RNA construct or the vector encoding the recombinant RNA construct, and wherein
(a) an expression level of the protein of interest is higher in the cell compared to the expression level of the protein of interest in a corresponding cell comprising a corresponding recombinant RNA construct or a vector encoding the recombinant RNA construct in which the at least one RNA sequence comprising the siRNA is upstream of the at least one RNA sequence comprising the mRNA; and
(b) an expression level of the target RNA is lower in the cell compared to the expression level of the target RNA in a corresponding cell comprising a corresponding recombinant RNA construct or a vector encoding the recombinant RNA construct in which the at least one RNA sequence comprising the siRNA is upstream of the at least one RNA sequence comprising the mRNA.

95. A pharmaceutical composition comprising the composition of claim 78 and a pharmaceutically acceptable excipient, carrier, or diluent.

96. A method of treating a disease or a condition in a human subject in need thereof, comprising administering to the human subject the pharmaceutical composition of claim 95, wherein the pharmaceutical composition comprises a therapeutically effective amount of the recombinant RNA construct or the vector encoding the recombinant RNA construct.

97. A method of modulating expression of two or more genes in a cell, comprising introducing to the cell a recombinant RNA construct or a vector encoding the recombinant RNA construct, wherein the recombinant RNA construct comprises:
(i) at least one RNA sequence comprising a small interfering RNA (siRNA) capable of binding to a target RNA; and
(ii) at least one RNA sequence comprising a messenger RNA (mRNA) encoding a protein of interest,
wherein the at least one RNA sequence comprising the mRNA is located upstream or downstream of the at least one RNA sequence comprising the siRNA; and
wherein the target RNA is different from the mRNA encoding the protein of interest.
